(19) United States
(12) Patent Application Publication
(10) Pub. No.: US 2025/0170130 A1
(43) Pub. Date: May 29, 2025

(54) MODULATORS OF MYC FAMILY PROTO-ONCOGENE PROTEIN

(71) Applicant: Nalo Therapeutics, Palo Alto, CA (US)

(72) Inventors: William Greenlee, Teaneck, NJ (US); Stephen J. Shuttleworth, Buckinghamshire (GB); Keith Wilson, Belmont, CA (US)

(21) Appl. No.: 18/840,640

(22) PCT Filed: Feb. 24, 2023
(86) PCT No.: PCT/US2023/063210

§ 371 Date: Aug. 22, 2024

Related U.S. Application Data
(60) Provisional application No. 63/313,905, filed on Feb. 25, 2022.

(30) Foreign Application Priority Data
Feb. 25, 2022 (WO) PCT/US2022/017863

(51) Int. Cl. A61K 31/506; C07D 401/14; C07D 403/12; C07D 403/14; C07D 413/14; C07D 471/10; C07D 487/04; C07D 487/10

(52) U.S. Cl. CPC A61K 31/506; C07D 401/14; C07D 403/12; C07D 403/14; C07D 413/14; C07D 471/10; C07D 487/04; C07D 487/10

(57) ABSTRACT

Disclosed herein are compounds and compositions having potency in the modulation of Myc family proteins. Such compounds and compositions can be used in the treatment of proliferative diseases, such as cancer, or in the treatment of disease where modulation of Myc family proteins is desired. Also disclosed herein are methods of using said compounds and compositions.

### CROSS REFERENCE TO RELATED APPLICATIONS

This application claims priority to and the benefit of U.S. Provisional Patent Application No. 63/313,905 filed Feb. 25, 2022, and PCT/US2022/017863 filed Feb. 25, 2022, each of which is incorporated herein by reference in its entirety.

### BACKGROUND

The MYC proto-oncogene family comprises three members: C-MYC, MYCN, and MYCL. These oncogenes encode c-Myc, N-Myc, and L-Myc oncoproteins, respectively, which belong to a family of “super-transcription factors” that regulate the transcription of more than 15% of the entire genome. Recent studies in mouse models have suggested that the regulation of oncogenic Myc proteins could potentially lead to the development of cancer therapeutics, as it has been demonstrated that even transient inactivation of Myc causes tumor regression. However, the development of drugs and therapeutics that directly targets Myc proteins has met with two major challenges. First, Myc proteins lack a well-defined active site for the binding of small molecules, thus providing challenges for the functional modulation or inhibition of their activities. Second, Myc proteins are predominantly located in cell nuclei, and targeting nuclear Myc proteins with antibodies can be technically challenging. These challenges have spawned strategies for indirect regulation of Myc proteins.

For example, amplification and overexpression of N-Myc can lead to tumorigenesis. Excess N-Myc is associated with a variety of tumors, e.g., neuroblastomas. MYCN can also be activated in tumors through somatic mutation.

C-Myc can also be constitutively expressed in various cancers such as cervix, colon, breast, lung and stomach cancers. Such constitutive expression can lead to increased expression of other genes that are involved in cell proliferation.

Amplification of the, e.g., N-Myc gene in patients frequently results in poor health outcomes. However, strategies for direct modulation of Myc proteins remain elusive, as the Myc proteins are not easily targeted.

Therefore, an ongoing need exists for small-molecule therapeutic modulators of Myc proteins for the treatment of various ailments, diseases and disorders, e.g., cancer.

### SUMMARY

The present disclosure provides compounds and compositions that are useful as Myc protein modulators, and methods of using the same. Furthermore, the present disclosure contemplates using disclosed compounds and compositions as direct modulators of Myc proteins in the treatment of proliferative disease, such as cancer, or in the treatment of diseases where modulation of Myc family proteins is desired.

For example, the present disclosure provides a compound of Formula I:

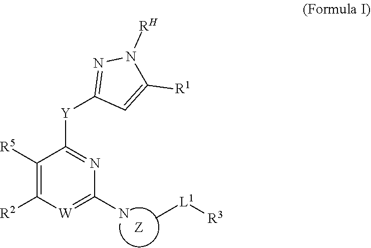

or a pharmaceutically acceptable salt, stereoisomer, and/or N-oxide thereof, wherein:
   W is selected from the group consisting of N, C—H, and C—F;
   Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
   ring Z is a 4-10 membered N-bound heterocyclic, wherein Z may optionally be substituted by one or two substituents each independently selected from the group consisting of halo, hydroxyl, C₁-C₄ alkyl (optionally substituted by one, two or three halogens), oxo, C₁-C₄ alkoxy, and cyano;
   L¹ is bond or C₁-C₆ alkylene (optionally substituted by one, two or three R⁴);
   R³ is selected from the group consisting of H, C₁-C₆ alkyl, NR′R′, S(O)₀₋₂C₁-C₄alkyl, SO₂N(R^{N})₂, N(R^{N})SO₂-C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
   R⁴ is each independently selected from the group consisting of halogen and C₁-C₆ alkyl, or two R⁴ together with the carbon from which the R⁴ are attached form a C₃-C₅ carbocyclyl;
   R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
   R^{H} is selected from the group consisting of H, C₁-C₃alkyl, —C(O)—C₁-C₃alkyl, and C(O)—O—C₁-C₃alkyl;
   R^{N} is selected from the group consisting of H and C₁-C₃alkyl, or two R^{N} together with the nitrogen atoms to which the R^{N}s are attached form a 4-6 membered heterocyclic ring;
   R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
   R² is selected from the group consisting of H, F, CF₃, —C(O)—O-methyl, —C(O)OH, —O—methyl, —O-ethyl, C₃-C₇ cycloalkyl, and heterocyclyl; and
   R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring;
   wherein when ring Z is a 6 membered monocyclic heterocycle, L¹ is C₁-C₆alkylene or L¹ is a bond and R³ is selected from C₂-C₆alkyl, SO₂C₁-C₄alkyl, and NHR′ wherein R′ is selected from the group consisting of C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo.

Exemplary contemplated compounds are represented by Formula VI:

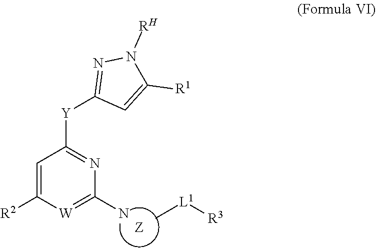

or a pharmaceutically acceptable salt, stereoisomer, and/or N-oxide thereof, wherein:
   W is selected from the group consisting of N, C—H, and C—F;
   Y is selected from the group consisting of NH, N—CH₃, O, S, CH₂, CF₂, CH(CH₃), C(CH₃)₂, and C(CH₂CH₂);
   ring Z is a 4-10 membered N-bound heterocyclic, wherein Z may optionally be substituted by one or two substituents each independently selected from the group consisting of halo, hydroxyl, C₁-C₄ alkyl (optionally substituted by one, two or three halogens), and oxo;
   L¹ is bond or C₁-C₆ alkylene (optionally substituted by one, two or three halogens);
   R³ is selected from the group consisting of H, C₁-C₆ alkyl, NR′R′, and heterocyclyl optionally substituted by one or two substituents each independently selected from the group consisting of halo, hydroxyl, C₁-C₄ alkyl (optionally substituted by one, two or three halogens), and oxo;
   R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
   R^{H} is selected from the group consisting of H, C₁-C₃alkyl, —C(O)—C₁-C₃alkyl, and C(O)—O—C₁-C₃alkyl;
   R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, heterocyclyl, or heteroaryl may be substituted by one, two or three substituents each independently selected from halo and C₁-C₄alkyl (optionally substituted by one, two or three halogens); and
   R² is selected from the group consisting of H, F, CF₃, —C(O)—O-methyl, —C(O)OH, —O—methyl, C₃-C₇ cycloalkyl, and heterocyclyl;
   wherein when ring Z is a 6 membered monocyclic heterocycle, L¹ is C₁-C₆alkylene or L¹ is a bond and R³ is selected from C₁-C₆alkyl and NHR′.

Also contemplated herein is a compound represented by Formula II:

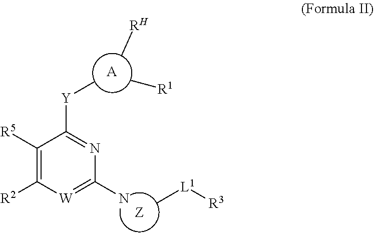

or a pharmaceutically acceptable salt, stereoisomer, and/or N-oxide thereof, wherein:
   W is selected from the group consisting of N, C—H, and C—F;
   Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
   ring A is a 5-6 membered heteroaryl optionally substituted with C₁-C₄ alkyl;
   ring Z is selected from the group consisting of 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, oxo, C₁-C₄alkoxy, and cyano;
   L¹ is bond or C₁-C₆ alkylene (optionally substituted by one, two or three R⁴);
   R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
   R⁴ is each independently selected from the group consisting of halogen and C₁-C₆ alkyl, or two R⁴ together with the carbon from which the R⁴ are attached form a C₃-C₅ carbocyclyl;
   R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
   R^{H} is selected from the group consisting of H, C₁-C₃alkyl, —C(O)—C₁-C₃alkyl, and C(O)—O—C₁-C₃alkyl;
   R^{N} is selected from the group consisting of H and C₁-C₃alkyl, or two R^{N} together with the nitrogen atoms to which the R^{N}s are attached form a 4-6 membered heterocyclic ring;
   R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
   R² is selected from the group consisting of H, F, CF₃, —C(O)—O-methyl, —C(O)OH, —O—methyl, —O-ethyl, C₃-C₇ cycloalkyl, and heterocyclyl; and
   R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring.

Exemplary contemplated compounds are represented by Formula III:

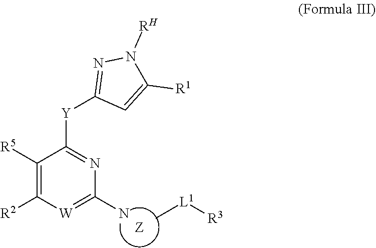

or a pharmaceutically acceptable salt, stereoisomer, and/or N-oxide thereof, wherein:
   W is selected from the group consisting of N, C—H, and C—F;
   Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
ring Z is selected from the group consisting of 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, oxo, C₁-C₄alkoxy, and cyano;
   L¹ is bond or C₁-C₆ alkylene (optionally substituted by one, two or three R⁴);
   R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
   R⁴ is each independently selected from the group consisting of halogen and C₁-C₆ alkyl, or two R⁴ together with the carbon from which the R⁴ are attached form a C₃-C₅ carbocyclyl;
   R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
   R^{H} is selected from the group consisting of H, C₁-C₃alkyl, —C(O)—C₁-C₃alkyl, and C(O)—O—C₁-C₃alkyl;
   R^{N} is selected from the group consisting of H and C₁-C₃alkyl, or two R^{N} together with the nitrogen atoms to which the R^{N}s are attached form a 4-6 membered heterocyclic ring;
   R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
   R² is selected from the group consisting of H, F, CF₃, —C(O)—O-methyl, —C(O)OH, —O—methyl, —O-ethyl, C₃-C₇ cycloalkyl, and heterocyclyl, and
   R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring.

Other contemplated compounds of the present disclosure include those represented by Formula IV:

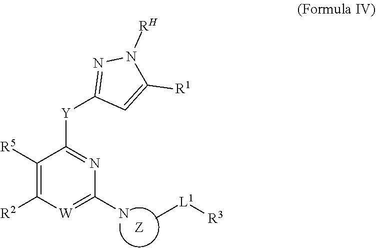

or a pharmaceutically acceptable salt, stereoisomer, and/or N-oxide thereof, wherein:
   W is selected from the group consisting of N, C—H, and C—F;
   Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
ring Z is a 4-10 membered N-bound heterocyclic, wherein Z may optionally be substituted by one or two substituents each independently selected from the group consisting of halo, hydroxyl, C₁-C₄ alkyl (optionally substituted by one, two or three halogens), oxo, C₁-C₄alkoxy, and cyano;
   L¹ is bond or C₁-C₆ alkylene (optionally substituted by one, two or three R⁴);
   R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
   R⁴ is each independently selected from the group consisting of halogen and C₁-C₆ alkyl, or two R⁴ together with the carbon from which the R⁴ are attached form a C₃-C₅ carbocyclyl;
   R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
   R^{H} is selected from the group consisting of H, C₁-C₃alkyl, —C(O)—C₁-C₃alkyl, and C(O)—O—C₁-C₃alkyl;
   R^{N} is selected from the group consisting of H and C₁-C₃alkyl, or two R^{N} together with the nitrogen atoms to which the R^{N}s are attached form a 4-6 membered heterocyclic ring;
   R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
   R² is selected from the group consisting of H, F, CF₃, —C(O)—O-methyl, —C(O)OH, —O—methyl, —O-ethyl, methyl, C₃-C₇ cycloalkyl, and heterocyclyl; and
   R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring.

Also contemplated herein are compound represented by Formula V:

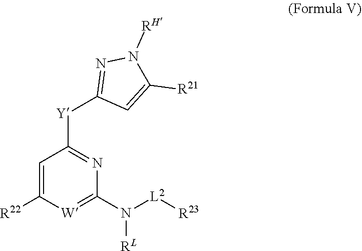

or a pharmaceutically acceptable salt, stereoisomer, and/or N-oxide thereof, wherein:
   W′ is selected from the group consisting of N, C—H, and C—F;
   Y′ is selected from the group consisting of NH, N—CH₃, O, S, CH₂, CFH, CF₂, CH(CH₃), C(CH₃)₂, C(CH₂CH₂). CHCF₃, CHCF₂H, CH(OCF₂H), CHOH, C(CH₃)OH, and CH(OCH₃);
   R^{L} is H or C₁-C₆ alkyl;
   L² is bond or C₁-C₆ alkylene (optionally substituted by one, two or three R²⁴);
   R²³ is NR′R′;
   R²⁴ is each independently selected from the group consisting of halogen and C₁-C₆ alkyl, or two R⁴ together with the carbon from which the R⁴ are attached form a C₃-C₅ carbocyclyl;
   R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo; or two R's together with the nitrogen to which they are attached form a heterocyclyl which may optionally be substituted by methyl, halo, cyano, oxo, or hydroxyl;
   R^{H′} is selected from the group consisting of H, C₁-C₃alkyl, —C(O)—C₁-C₃alkyl, and C(O)—O—C₁-C₃alkyl;
   R²¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens); and
   R²² is selected from the group consisting of H, F, —C(O)—O-methyl, —C(O)OH, —O—methyl, —O-ethyl, methyl, C₃-C₇ cycloalkyl, and heterocyclyl.

Pharmaceutical compositions comprising a disclosed compound or a pharmaceutically acceptable salt, stereoisomer, and/or N-oxide thereof, as described herein, for example a disclosed pharmaceutical composition may include least one or more pharmaceutically acceptable carriers, diluents, stabilizers, excipients, dispersing agents, suspending agents, and/or thickening agents. The present disclosure also provides a method of manufacturing of the compounds described herein, or a pharmaceutically acceptable salt, stereoisomer, and/or N-oxide thereof.

A method of modulating the amount and activity of a Myc family protein (i.e., C-Myc, N-Myc, L-Myc, or human Myc) is also provided, for example, an activity of a Myc family protein may be modulated in a cell by contacting a cell with an effective amount of a compound as described herein, or a pharmaceutically acceptable salt, stereoisomer, and/or N-oxide thereof.

The present disclosure also provides a method of treating a Myc family protein associated disease in a subject in need thereof, the method comprising administering a therapeutically effective amount of a compound described herein, or a pharmaceutically acceptable salt, stereoisomer, and/or N-oxide thereof, including embodiments in any examples, tables, or figures. In some embodiments, the subject is a human subject and the disease is a proliferative disease, such as cancer.

### DETAILED DESCRIPTION

### Definitions

Unless defined otherwise, all technical and scientific terms used herein have the same meaning as commonly understood by one of ordinary skill in the art to which this disclosure pertains.

It is understood that the definitions provided herein are not intended to be mutually exclusive. Accordingly, some chemical moieties may fall within the definition of more than one term.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, n-butoxy, tert-butoxy, sec-butoxy, n-pentoxy, n-hexoxy, 1,2-dimethylbutoxy, etc.

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

As used herein, the term “alkylene” refers to a di-radical alkyl group. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

As used herein, the terms “alkenylene,” “alkynylene,” “arylene,” “arylalkylene,” and “alkylarylene” refer to di-radical alkenyl, alkynyl, aryl, arylalkyl, and alkylaryl groups, respectively.

As used herein, the term “azido” refers to group —N₃.

As used herein, the term “carboxyl,” “carboxy” or “carboxylate” refers to —CO₂H or salts thereof.

As used herein, the term “carbamoyl” refers to the group NH₂CO—.

The terms “cycloalkyl” or a “carbocyclic group” as used herein refers to a saturated or partially unsaturated hydrocarbon group of, for example, 3-10, 3-6, or 4-6 carbons, referred to herein as C₃₋₁₀cycloalkyl, or C₄₋₆cycloalkyl, respectively, and which may be monocyclic or bicyclic ring structures, e.g. 4-9 or 4-6 membered saturated ring structures, including bridged, fused or spirocyclic rings. Exemplary cycloalkyl groups include, but are not limited to, adamantanyl, cyclohexyl, cyclopentyl, cyclopentenyl, cyclobutyl, cyclopropyl, and indanyl.

As used herein, the groups

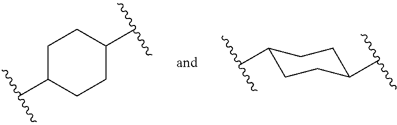 [SMILES: *C1CCC(*)CC1 ; *C1CCC(*)CC1]

are used interchangeably and refer to a cyclohexyl group.

As used herein, the term “cyano” and “carbonitrile” refer to the group —CN.

As used herein, the term “formyl” refers to the group —C(O)H.

As used herein, the term “guanidino” refers to the group —NHC(═NH)NH₂.

As used herein, the terms “halo” and “halogen” are used in the conventional sense to refer to a chloro, bromo, fluoro or iodo substituent.

As used herein, the terms “hydroxy” and “hydroxyl” refer to the group —OH.

The terms “heteroaryl” or “heteroaromatic group” as used herein refers to a monocyclic aromatic 5-6 membered ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridine or pyrimidine etc.

The terms “heterocyclyl” or “heterocyclic group” are art-recognized and refer to e.g. saturated or partially unsaturated, 4-10 membered monocyclic or bicyclic ring structures, or e.g. 4-9 or 4-6 membered saturated ring structures, including bridged, fused or spirocyclic rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran etc.

As used herein, the term “nitro” refers to the group —NO₂.

As used herein, the term “oxo” refers to the group (═O) or (O).

As used herein, the term “isomers” refers to compounds comprising the same numbers and types of atoms or components, but with different structural arrangement and connectivity of the atoms.

As used herein, the term “tautomer” refers to one of two or more structural isomers which readily convert from one isomeric form to another and which exist in equilibrium.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. Substituents around a carbocyclic or heterocyclic ring may be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the present disclosure embrace both solvated and unsolvated forms. In one embodiment, a disclosed compound is amorphous. In one embodiment, a disclosed compound is a single polymorph. In another embodiment, a disclosed compound is a mixture of polymorphs. In another embodiment, a disclosed compound is in a crystalline form.

The present disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in the examples herein by substituting an isotopically labeled reagent for a non-isotopically labeled reagent.

As used herein, singular articles such as “a,” “an” and “the” and similar referents in the context of describing the elements are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (i.e.,“such as”) provided herein, is intended merely to better illuminate the embodiments and does not pose a limitation on the scope of the claims unless otherwise stated.

In some embodiments, where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remains operable. Moreover, two or more steps or actions can be conducted simultaneously.

As used herein, a dash (“−”) that is not between two letters or symbols refers to a point of bonding or attachment for a substituent. For example, —NH₂ is attached through the nitrogen atom.

As used herein, the terms “active agent,” “drug,” “pharmacologically active agent” and “active pharmaceutical ingredient” are used interchangeably to refer to a compound or composition which, when administered to a subject, induces a desired pharmacologic or physiologic effect by local or systemic action or both.

As used herein, the term “prodrug” refers to compounds that are transformed in vivo to provide a compound or pharmaceutically acceptable salt, hydrate or solvate of the compound described herein. The transformation can occur by various mechanisms (i.e., esterase, amidase, phosphatase, oxidative and/or reductive metabolism) in various locations (i.e., in the intestinal lumen or upon transit into the intestine, blood, or liver).

As used herein, the term “modulator” refers to a compound or composition that increases or decreases the level of a target or the function of a target, which may be, but is not limited to, a Myc family protein, such as c-Myc, N-Myc, L-Myc and human Myc.

As used herein, the term “degrader” refers to a compound or composition that decreases the amount of a target or the activity of a target. In some embodiments, the target may be, but is not limited to, a Myc family protein comprising c-Myc, N-Myc, L-Myc and human Myc.

As used herein, the term “degrading” refers to a method or process that decreases the amount of a target or the activity of a target. In some embodiments, the target may be, but is not limited to, a Myc family protein comprising c-Myc, N-Myc, L-Myc and human Myc.

As used herein, the term “Myc family protein” refers to any one of the proteins c-Myc, N-Myc, or L-Myc as described herein. In some embodiments, a Myc protein is a c-Myc protein. In some embodiments, a Myc protein is a N-Myc protein. In some embodiments, a Myc protein is a L-Myc protein. In some embodiments, a Myc protein is a human c-Myc protein. In some embodiments, a Myc protein is a human N-Myc protein. In some embodiments, a Myc protein is a human L-Myc protein. In some embodiments, a Myc family protein is a human Myc family protein.

As used herein, the terms “N-Myc” and “MycN” can be used interchangeably and refer to the protein “V-Myc myelocytomatosis viral related oncogene, neuroblastoma derived” and include the wildtype and mutant forms of the protein. In some embodiments, MycN refers to the protein associated with one or more of database entries of Entrez Gene 4613, OMIM 164840, UniProt P04198, and RegSeq NP_005369.

As used herein, the term “c-Myc” refers to the protein “V-Myc myelocytomatosis viral oncogene” and include the wildtype and mutant forms of the protein. In some embodiments, c-Myc refers to the protein associated with one or more of database entries of Entrez Gene 4609, OMIM 190080, UniProt P01106, and RegSeq NP_002458.

As used herein, the term “L-Myc” refers to the protein “V-Myc myelocytomatosis viral oncogene homolog, lung carcinoma derived” and include the wildtype and mutant forms of the protein. In some embodiments, L-Myc refers to the protein associated with one or more of database entries of Entrez Gene 4610, OMIM 164850, UniProt P12524, and RegSeq NP_001028253.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines, felines, equines, bovines, ovines, rodentia, etc. and primates, i.e., non-human primates, and humans. Non-human animal models, i.e., mammals, non-human primates, murines, lagomorpha, etc. may be used for experimental investigations.

As used herein, the terms “treating,” “treatment,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human and includes: (a) preventing the disease or a symptom of a disease from occurring in a subject which may be predisposed to the disease but has not yet been diagnosed as having it (i.e., including diseases that may be associated with or caused by a primary disease); (b) inhibiting the disease, i.e., arresting its development; and (c) relieving the disease, i.e., causing regression of the disease (i.e., reduction in of tumor burden). In some embodiments, certain methods described herein treat cancer associated with the signaling pathway of a Myc family protein, such as c-Myc, N-Myc, L-Myc or human Myc.

As used herein, the term “pharmaceutically acceptable salt” refers to a salt which is acceptable for administration to a subject. It is understood that such salts, with counter ions, will have acceptable mammalian safety for a given dosage regime. Such salts can also be derived from pharmaceutically acceptable inorganic or organic bases and from pharmaceutically acceptable inorganic or organic acids, and may comprise organic and inorganic counter ions. The neutral forms of the compounds described herein may be converted to the corresponding salt forms by contacting the compound with a base or acid and isolating the resulting salts.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like.

Other examples of salts include anions of the compounds of the present disclosure compounded with a suitable cation such as N⁺, NH₄⁺, and NW₄⁺ (where W can be a C₁-C₈alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds included in the present compositions that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

As used herein, the terms “determining,” “measuring,” “assessing,” and “assaying” are used interchangeably and include both quantitative and qualitative determinations.

As used herein, the phrase “signaling pathway” refers to a series of interactions between cellular components, both intracellular and extracellular, that conveys a change to one or more other components in a living organism, which may cause a subsequent change to additional component. Optionally, the changes conveyed by one signaling pathway may propagate to other signaling pathway components. Examples of cellular components include, but are not limited to, proteins, nucleic acids, peptides, lipids and small molecules.

As used herein, the terms “effective amount” and “therapeutically effective amount” are used interchangeably and refer to the amount of a compound that, when administered to a mammal or other subject for treating a disease, condition, or disorder, is sufficient to affect such treatment for the disease, condition, or disorder. The “effective amount” or “therapeutically effective amount” will vary depending on the compound, the disease and its severity and the age, weight, etc., of the subject to be treated.

As used herein, the terms “pharmaceutically acceptable excipient,” “pharmaceutically acceptable diluent,” “pharmaceutically acceptable carrier,” and “pharmaceutically acceptable adjuvant” refer to an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. The phrase “a pharmaceutically acceptable excipient, diluent, carrier and adjuvant” as used in the specification and claims includes both one and more than one such excipient, diluent, carrier, and adjuvant.

As used herein, the term “pharmaceutical composition” is meant to encompass a composition suitable for administration to a subject, such as a mammal, especially a human. In general a “pharmaceutical composition” is sterile, and free of contaminants that are capable of eliciting an undesirable response within the subject (i.e., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

Unless the specific stereochemistry is expressly indicated, all chiral, diastereomeric, and racemic forms of a compound are intended. Thus, compounds described herein include enriched or resolved optical isomers at any or all asymmetric atoms as are apparent from the depictions. Racemic mixtures of (R)-enantiomer and (S)-enantiomer, and enantio-enriched stereomeric mixtures comprising of (R)- and (S)-enantiomers, as well as the individual optical isomers can be isolated or synthesized so as to be substantially free of their enantiomeric or diastereomeric partners, and these stereoisomers are all within the scope of the present technology.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates, among others. The identification and preparation of any particular solvate is within the skill of the ordinary artisan of synthetic organic or medicinal chemistry.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### Compounds

The disclosure is generally directed to compounds that modulate (e.g., degrade) MycN and/or MycC, and may therefore have significant antineoplastic properties. The disclosed compounds and pharmaceutical compositions thereof find use in a variety of applications in which the modulation of the amount and activity of a Myc protein is desired, including use as potent antineoplastic agents.

Thus provided herein, in part, is a compound of Formula I:

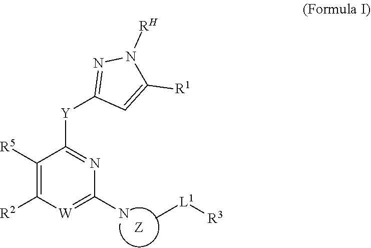

or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
ring Z is a 4-10 membered N-bound heterocyclic, wherein Z may optionally be substituted by one or two substituents each independently selected from the group consisting of halo, hydroxyl, C₁-C₄ alkyl (optionally substituted by one, two or three halogens), oxo, C₁-C₄ alkoxy, and cyano;
   L¹ is bond or C₁-C₆ alkylene (optionally substituted by one, two or three R⁴);
   R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
   R⁴ is each independently selected from the group consisting of halogen and C₁-C₆ alkyl, or two R⁴ together with the carbon from which the R⁴ are attached form a C₃-C₅ carbocyclyl;
   R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
   R^{H} is selected from the group consisting of H, C₁-C₃alkyl, —C(O)—C₁-C₃alkyl, and C(O)—O—C₁-C₃alkyl;
   R^{N} is selected from the group consisting of H and C₁-C₃alkyl, or two R^{N} together with the nitrogen atoms to which the R^{N}s are attached form a 4-6 membered heterocyclic ring;
   R¹ is selected from the group consisting of C₃-C₁₁ cycloalkyl, spiro C₅-C₁₀ bicycloalkyl, heterocyclyl, cyano, halo, and heteroaryl; wherein C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
   R² is selected from the group consisting of H, F, CF₃, —C(O)—O-methyl, —C(O)OH, —O—methyl, —O-ethyl, C₃-C₇ cycloalkyl, and heterocyclyl; and
   R⁵ is selected from the group consisting of H, halo, C₁-C₄ alkyl (optionally substituted by one, two or three halogens); or R² and R⁵ together with the carbon atoms to which R² and R₅ are attached form a 5-6 membered carbocyclic ring;
wherein when ring Z is a 6 membered monocyclic heterocycle, L¹ is C₁-C₆alkylene or L¹ is a bond and R³ is selected from C₂-C₆alkyl, SO₂C₁-C₄alkyl, and NHR′ wherein R′ is selected from the group consisting of C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo.

For example, Formula I may be provided as:

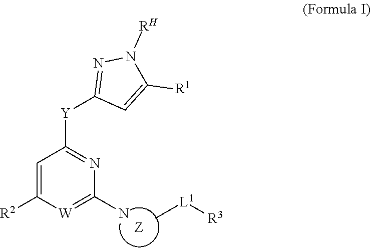

or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CF₂, CH(CH₃), C(CH₃)₂, and C(CH₂CH₂);
ring Z is a 4-10 membered N-bound heterocyclic, wherein Z may optionally be substituted by one or two substituents each independently selected from the group consisting of halo, hydroxyl, C₁-C₄ alkyl (optionally substituted by one, two or three halogens), and oxo;
L¹ is bond or C₁-C₆ alkylene (optionally substituted by one, two or three halogens);
R³ is selected from the group consisting of H, C₁-C₆ alkyl, NR′R′, and heterocyclyl optionally substituted by one or two substituents each independently selected from the group consisting of halo, hydroxyl, C₁-C₄ alkyl (optionally substituted by one, two or three halogens), and oxo;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R¹ is selected from the group consisting of C₃-C₁₀ cycloalkyl, spiro C₅-C₁₀ bicycloalkyl, heterocyclyl, cyano, halo, and heteroaryl; wherein C₁-C₆ alkyl, C₃-C₇ cycloalkyl, heterocyclyl, or heteroaryl may be substituted by one, two or three substituents each independently selected from halo and C₁-C₄alkyl (optionally substituted by one, two or three halogens); and
R² is selected from the group consisting of H, F, CF₃, —C(O)—O-methyl, —C(O)OH, —O-methyl, C₃-C₇ cycloalkyl, and heterocyclyl;
wherein when ring Z is a 6 membered monocyclic heterocycle, L¹ is C₁-C₆alkylene or L¹ is a bond and R³ is selected from C₁-C₆alkyl and NHR′; or may be provided as:

In some embodiments, provided herein is a compound of Formula VI:

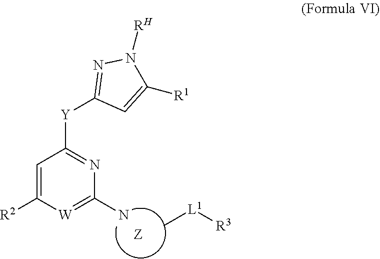

or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CF₂, CH(CH₃), C(CH₃)₂, and C(CH₂CH₂);
ring Z is a 4-10 membered N-bound heterocyclic, wherein Z may optionally be substituted by one or two substituents each independently selected from the group consisting of halo, hydroxyl, C₁-C₄ alkyl (optionally substituted by one, two or three halogens), and oxo;
L¹ is bond or C₁-C₆ alkylene (optionally substituted by one, two or three halogens);
R³ is selected from the group consisting of H, C₁-C₆ alkyl, NR′R′, and heterocyclyl optionally substituted by one or two substituents each independently selected from the group consisting of halo, hydroxyl, C₁-C₄ alkyl (optionally substituted by one, two or three halogens), and oxo;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, heterocyclyl, or heteroaryl may be substituted by one, two or three substituents each independently selected from halo and C₁-C₄alkyl (optionally substituted by one, two or three halogens); and
R² is selected from the group consisting of H, F, CF₃, —C(O)—O-methyl, —C(O)OH, —O-methyl, C₃-C₇ cycloalkyl, and heterocyclyl;
wherein when ring Z is a 6 membered monocyclic heterocycle, L¹ is C₁-C₆alkylene or L¹ is a bond and R³ is selected from C₁-C₆alkyl and NHR′.

In some embodiments, W is C—H. In other embodiments, W is C—F. In other embodiments, W is N.

In some embodiments, R^{H} is selected from the group consisting of H, —C(O)—C₁-C₃alkyl, and C(O)—O—C₁-C₃alkyl. In some embodiments, R^{H} is H.

In some embodiments, for example, W is N, and the compound is represented by the Formula Ia:

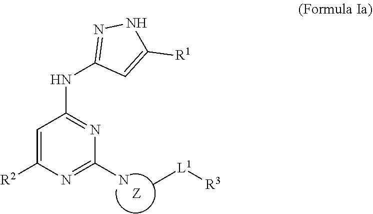

or a pharmaceutically acceptable salt, stereoisomer and/or N-oxide thereof.

In some embodiments, R¹ is a 5-6 membered heterocyclyl or C₃-C₆ cycloalkyl. In some embodiments, R¹ is a C₃-C₆ cycloalkyl. For example, R¹ may be cyclopropyl or cyclopentyl.

In some embodiments, ring Z is selected from the group consisting of 4-6 membered monocyclic heterocycle, 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, and oxo.

In some embodiments, ring Z is selected from the group consisting of 4-6 membered monocyclic heterocycle, 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), and oxo.

In some embodiments, ring Z is selected from the group consisting of 6-10 membered spiroheterocycle, a 6-10 membered fused bicyclic heterocyclic, and a 6-10 membered bridged cycloheteroalkyl, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, and oxo.

In some embodiments, ring Z is selected from the group consisting of 6-10 membered spiroheterocycle, a 6-10 membered fused bicyclic heterocyclic, and a 6-10 membered bridged cycloheteroalkyl, wherein Z may optionally be substituted by one or two substituents each independently selected from the group consisting of halo, hydroxyl, C₁-C₄ alkyl (optionally substituted by one, two or three halogens), and oxo.

In some embodiments, ring Z is selected from the group consisting of:

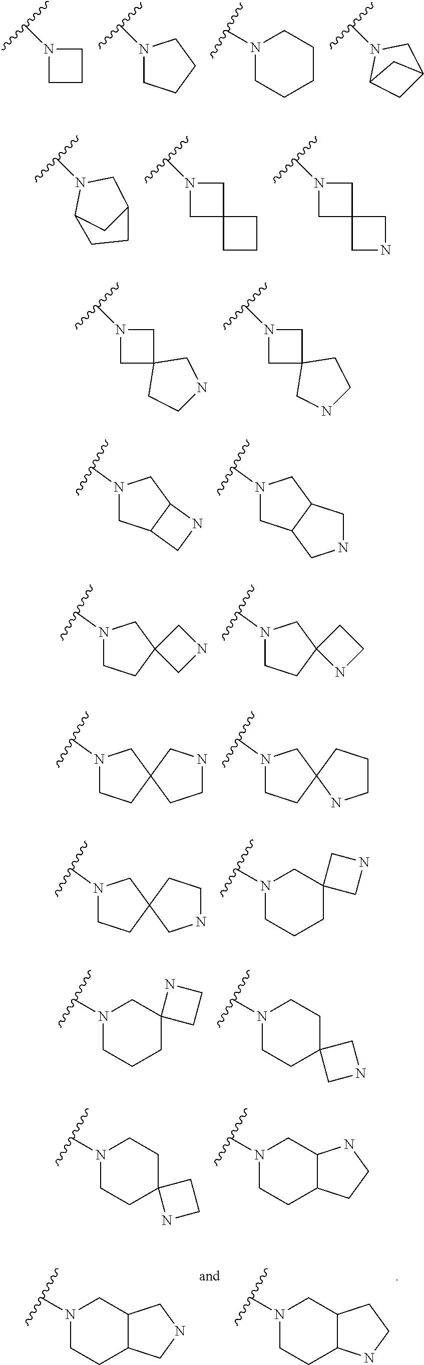

In some embodiments, ring Z is selected from the group consisting of:

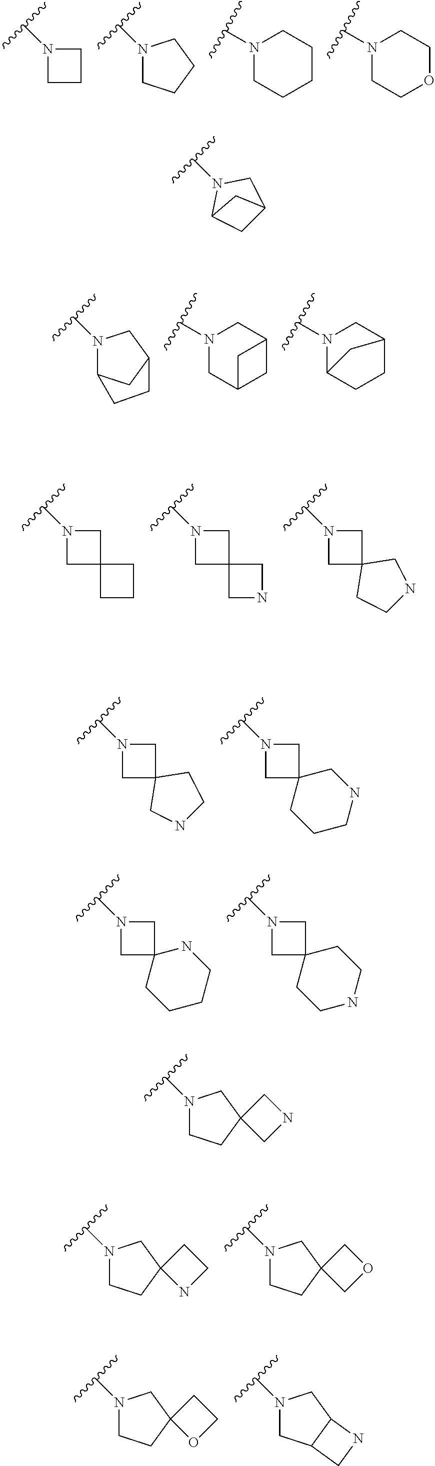

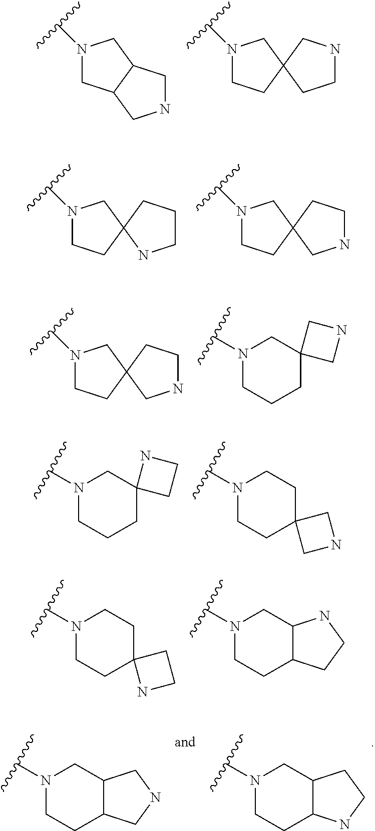

In some embodiments, ring Z is selected from the group consisting of:

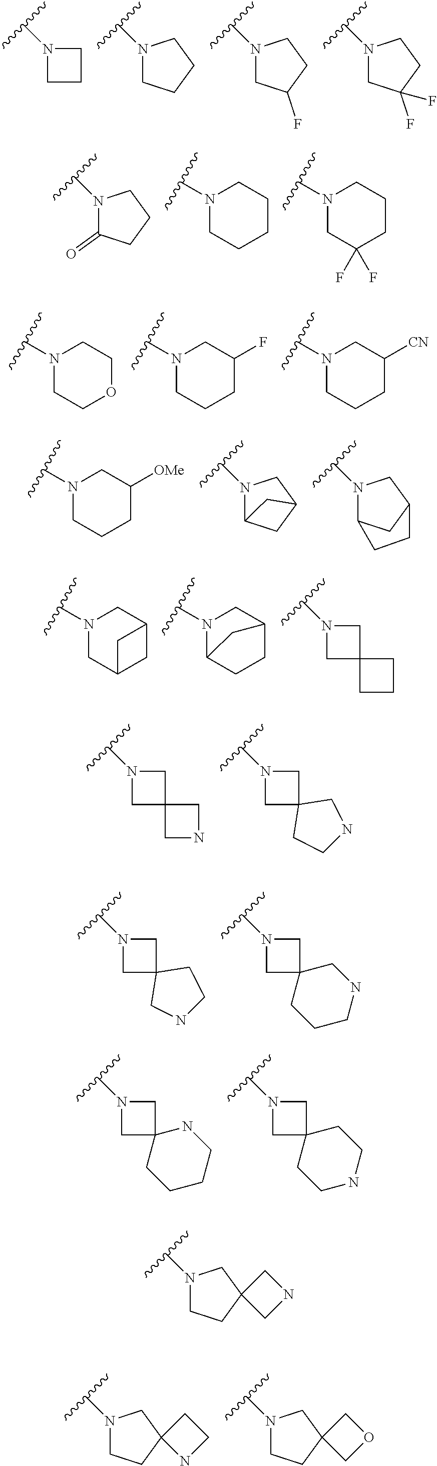

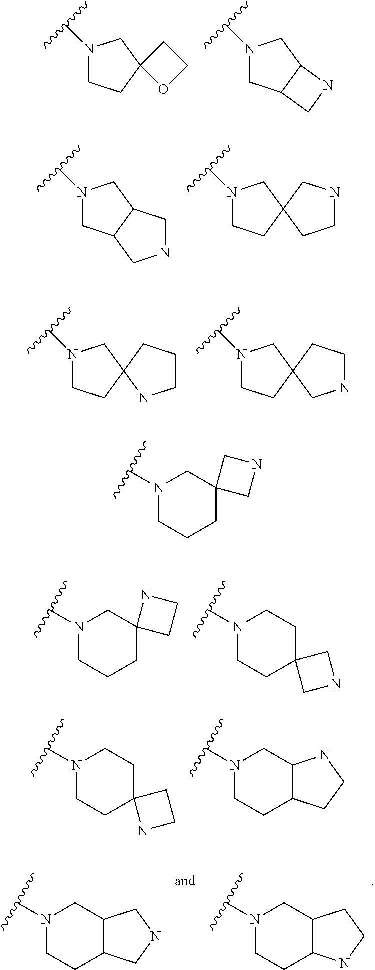

Exemplary disclosed compounds are represented by Formula Ib:

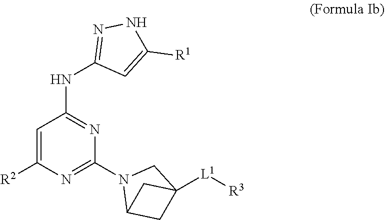

or a pharmaceutically acceptable salt, stereoisomer, and/or N-oxide thereof.

Other contemplated compounds provided herein are represented by Formula Ic:

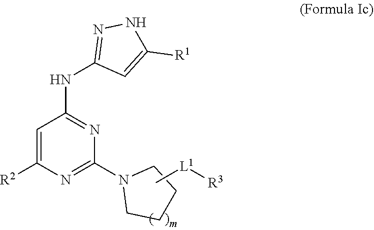

or a pharmaceutically acceptable salt, stereoisomer, and/or N-oxide thereof, wherein m is 0, 1, or 2.

Other contemplated compounds provided herein are represented by Formula Id:

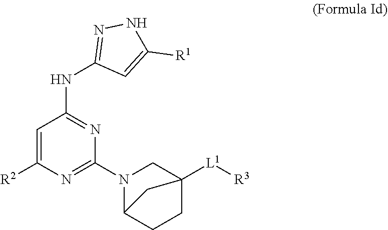

or a pharmaceutically acceptable salt, stereoisomer, and/or N-oxide thereof.

In some embodiments, also provided herein are compounds represented by Formula Ie:

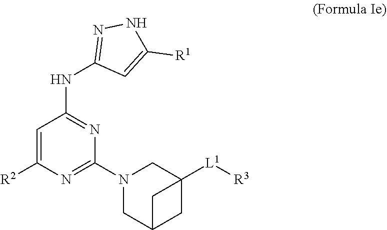

or a pharmaceutically acceptable salt, stereoisomer, and/or N-oxide thereof.

In some embodiments, L¹ is C₁-C₆ alkylene. For example, L¹ may be methylene, ethylene, or propylene. In some embodiments, L¹ is methylene.

In some embodiments, L¹ is C₁-C₄ alkylene substituted by one or two R⁴. For example, L¹ may be methylene substituted by one or two R⁴. In some embodiments, L¹ is C₁-C₄ alkylene substituted by one R⁴. In some embodiments, L¹ is C₁-C₄ alkylene substituted by two R⁴s. In some embodiments, R⁴ is C₁-C₃ alkyl (optionally substituted by one, two or three halogens). For example, R⁴ may each be independently selected from the group consisting of methyl, ethyl, and propyl. In other embodiments, two R⁴s together with the carbon to which the R⁴ are attached form a C₃-C₄ carbocyclyl. For example, two R⁴s together with the carbon to which the R⁴ are attached may form a cyclopropyl.

In other embodiments, L¹ is bond.

In some embodiments, R³ is H or C₁-C₆ alkyl. For example, R³ may be H or methyl. In some embodiments, R³ is H. In some embodiments, R³ is methyl.

In other embodiments, R³ is NR′R′, wherein R′ is each independently H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NR′R′, wherein R′ is each independently H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl. In some embodiments, R³ is NR′R′, wherein R′ is each independently H, C₁-C₆ alkyl (optionally substituted by one, two or three halogens). For example, R³ may be NH₂, NH(CH₃), or N(CH₃)₂.

In some embodiments, R³ is NH′R′ wherein R′ is H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NH′R′ wherein R′ is H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl.

In some embodiments, R³ is NH′R′ wherein R′ is C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NH′R′ wherein R′ is C₁-C₆ alkyl (optionally substituted by one, two or three halogens), C₁-C₆ alkylene-O—C₁-C₄ alkyl, or C₁-C₆ alkylene-SO₂—C₁-C₄ alkyl.

In other embodiments, R³ is 4-6 membered heterocyclyl optionally substituted by one or two substituents each independently selected from the group consisting of halo, hydroxyl, C₁-C₄ alkyl (optionally substituted by one, two or three halogens), and oxo. In some embodiments, R³ is 4-6 membered heterocyclyl comprising at least one nitrogen, wherein the heterocyclyl is optionally substituted by one or two substituents each independently selected from the group consisting of halo, hydroxyl, C₁-C₄ alkyl (optionally substituted by one, two or three halogens), and oxo. In some embodiments, R³ is a 4-membered heterocyclyl comprising at least one nitrogen, wherein the heterocyclyl is optionally substituted by one or two substituents each independently selected from the group consisting of halo, hydroxyl, C₁-C₄ alkyl (optionally substituted by one, two or three halogens), and oxo.

In some embodiments, R² is selected from the group consisting of H, F, CF₃, —C(O)—O-methyl, —C(O)OH, —O-methyl, C₃-C₇ cycloalkyl, and 4-6 membered heterocyclyl. In some embodiments, R² is selected from the group consisting of H, F, CF₃, —C(O)—O-methyl, —C(O)OH, —O-methyl, and C₃-C₇ cycloalkyl. In some embodiments, R² is selected from the group consisting of H, F, —O-methyl, and 4-membered heterocyclyl. In some embodiments, R² is selected from the group consisting of H, F, CF₃, —C(O)—O-methyl, —C(O)OH, and —O—methyl. In some embodiments, R² is H or F. In some embodiments, R² is H.

In some embodiments, R⁵ is H or methyl. In some embodiments, R⁵ is methyl. In some embodiments, R⁵ is H. In other embodiments, R² and R⁵ together with the carbon atoms to which R² and R⁵ are attached form a 5-membered carbocyclic ring.

A contemplated compound, for example, may be selected from the group consisting of:

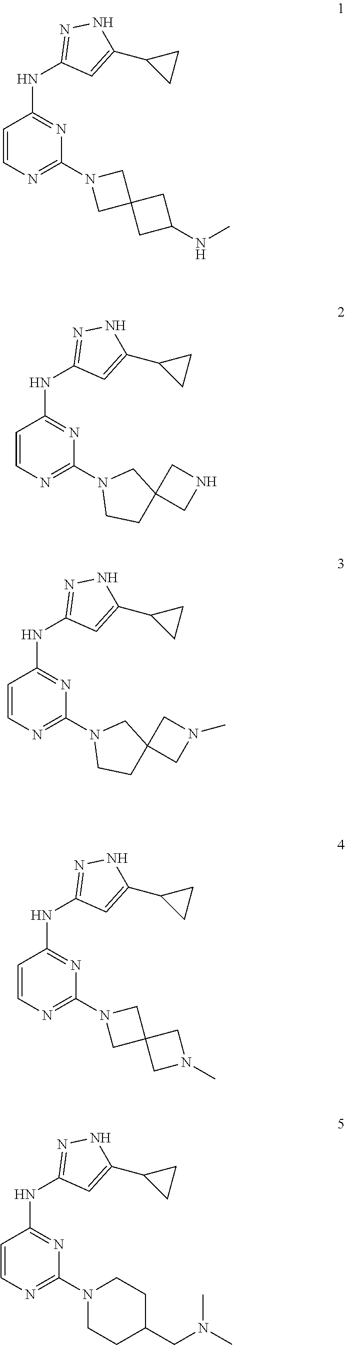 [SMILES: CNC1CC2(C1)CN(c1nccc(Nc3cc(C4CC4)[nH]n3)n1)C2 ; c1cc(Nc2cc(C3CC3)[nH]n2)nc(N2CCC3(CNC3)C2)n1 ; CN1CC2(CCN(c3nccc(Nc4cc(C5CC5)[nH]n4)n3)C2)C1 ; CN1CC2(C1)CN(c1nccc(Nc3cc(C4CC4)[nH]n3)n1)C2 ; CN(C)CC1CCN(c2nccc(Nc3cc(C4CC4)[nH]n3)n2)CC1]

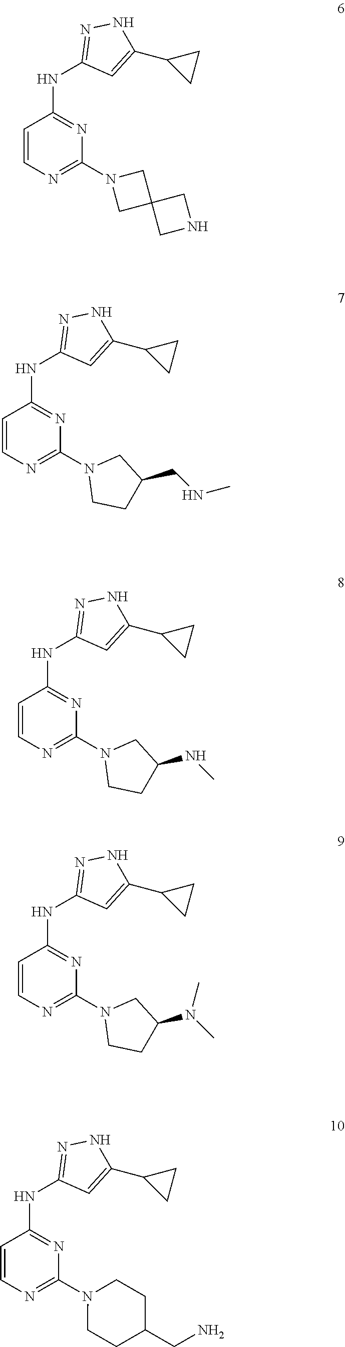 [SMILES: c1cc(Nc2cc(C3CC3)[nH]n2)nc(N2CC3(CNC3)C2)n1 ; CNC[C@H]1CCN(c2nccc(Nc3cc(C4CC4)[nH]n3)n2)C1 ; CN[C@H]1CCN(c2nccc(Nc3cc(C4CC4)[nH]n3)n2)C1 ; CN(C)[C@H]1CCN(c2nccc(Nc3cc(C4CC4)[nH]n3)n2)C1 ; NCC1CCN(c2nccc(Nc3cc(C4CC4)[nH]n3)n2)CC1]

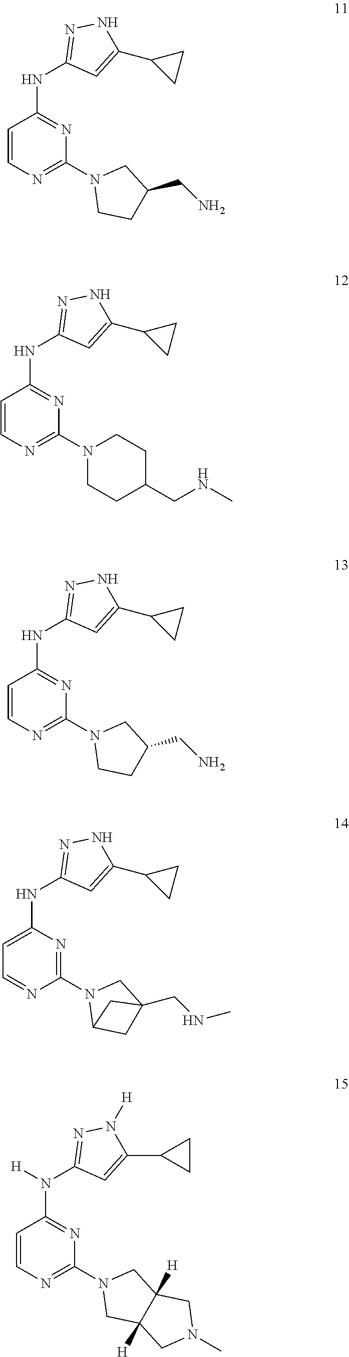 [SMILES: NC[C@H]1CCN(c2nccc(Nc3cc(C4CC4)[nH]n3)n2)C1 ; CNCC1CCN(c2nccc(Nc3cc(C4CC4)[nH]n3)n2)CC1 ; NC[C@@H]1CCN(c2nccc(Nc3cc(C4CC4)[nH]n3)n2)C1 ; CNCC12CC(C1)N(c1nccc(Nc3cc(C4CC4)[nH]n3)n1)C2 ; CN1C[C@@H]2CN(c3nccc(Nc4cc(C5CC5)[nH]n4)n3)C[C@@H]2C1]

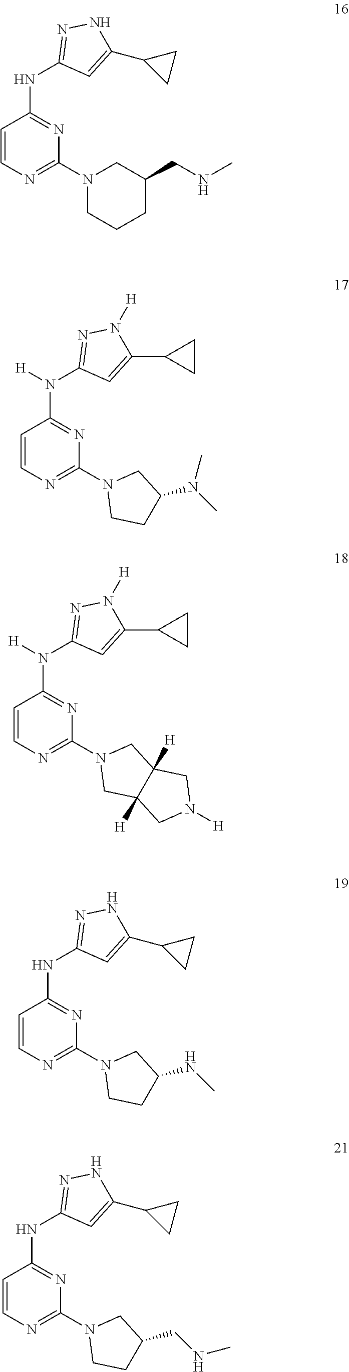 [SMILES: CNC[C@H]1CCCN(c2nccc(Nc3cc(C4CC4)[nH]n3)n2)C1 ; CN(C)[C@@H]1CCN(c2nccc(Nc3cc(C4CC4)[nH]n3)n2)C1 ; c1cc(Nc2cc(C3CC3)[nH]n2)nc(N2C[C@H]3CNC[C@H]3C2)n1 ; CN[C@@H]1CCN(c2nccc(Nc3cc(C4CC4)[nH]n3)n2)C1 ; CNC[C@@H]1CCN(c2nccc(Nc3cc(C4CC4)[nH]n3)n2)C1]

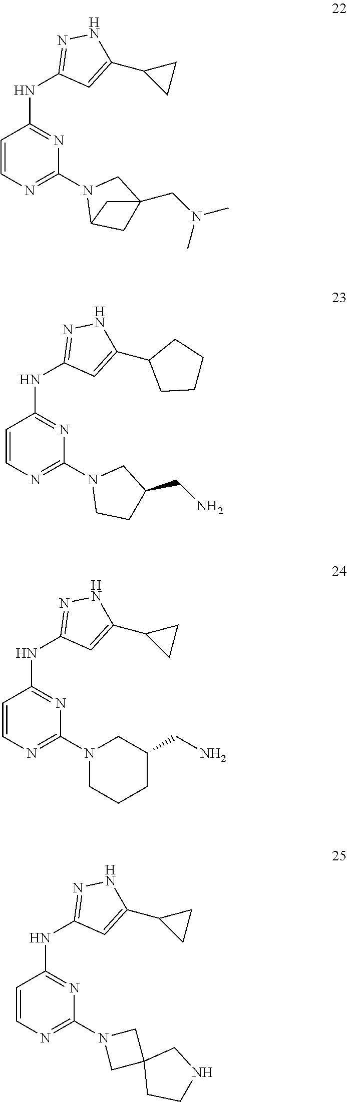 [SMILES: CN(C)CC12CC(C1)N(c1nccc(Nc3cc(C4CC4)[nH]n3)n1)C2 ; NC[C@H]1CCN(c2nccc(Nc3cc(C4CCCC4)[nH]n3)n2)C1 ; NC[C@@H]1CCCN(c2nccc(Nc3cc(C4CC4)[nH]n3)n2)C1 ; c1cc(Nc2cc(C3CC3)[nH]n2)nc(N2CC3(CCNC3)C2)n1]

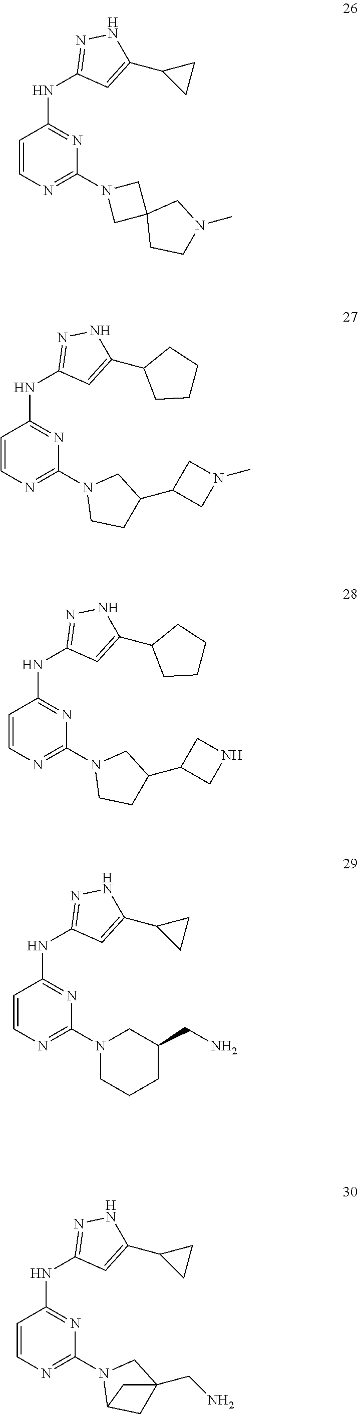 [SMILES: CN1CCC2(C1)CN(c1nccc(Nc3cc(C4CC4)[nH]n3)n1)C2 ; CN1CC(C2CCN(c3nccc(Nc4cc(C5CCCC5)[nH]n4)n3)C2)C1 ; c1cc(Nc2cc(C3CCCC3)[nH]n2)nc(N2CCC(C3CNC3)C2)n1 ; NC[C@H]1CCCN(c2nccc(Nc3cc(C4CC4)[nH]n3)n2)C1 ; NCC12CC(C1)N(c1nccc(Nc3cc(C4CC4)[nH]n3)n1)C2]

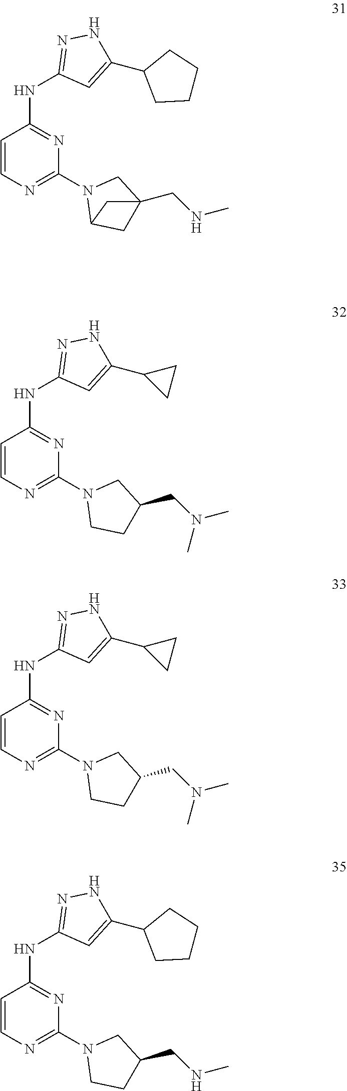 [SMILES: CNCC12CC(C1)N(c1nccc(Nc3cc(C4CCCC4)[nH]n3)n1)C2 ; CN(C)C[C@H]1CCN(c2nccc(Nc3cc(C4CC4)[nH]n3)n2)C1 ; CN(C)C[C@@H]1CCN(c2nccc(Nc3cc(C4CC4)[nH]n3)n2)C1 ; CNC[C@H]1CCN(c2nccc(Nc3cc(C4CCCC4)[nH]n3)n2)C1]

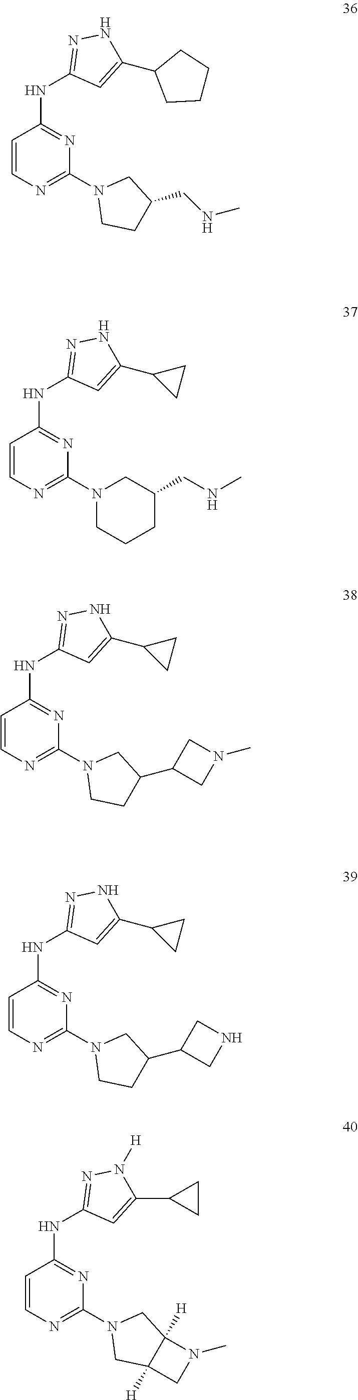 [SMILES: CNC[C@@H]1CCN(c2nccc(Nc3cc(C4CCCC4)[nH]n3)n2)C1 ; CNC[C@@H]1CCCN(c2nccc(Nc3cc(C4CC4)[nH]n3)n2)C1 ; CN1CC(C2CCN(c3nccc(Nc4cc(C5CC5)[nH]n4)n3)C2)C1 ; c1cc(Nc2cc(C3CC3)[nH]n2)nc(N2CCC(C3CNC3)C2)n1 ; CN1C[C@H]2CN(c3nccc(Nc4cc(C5CC5)[nH]n4)n3)C[C@H]21]

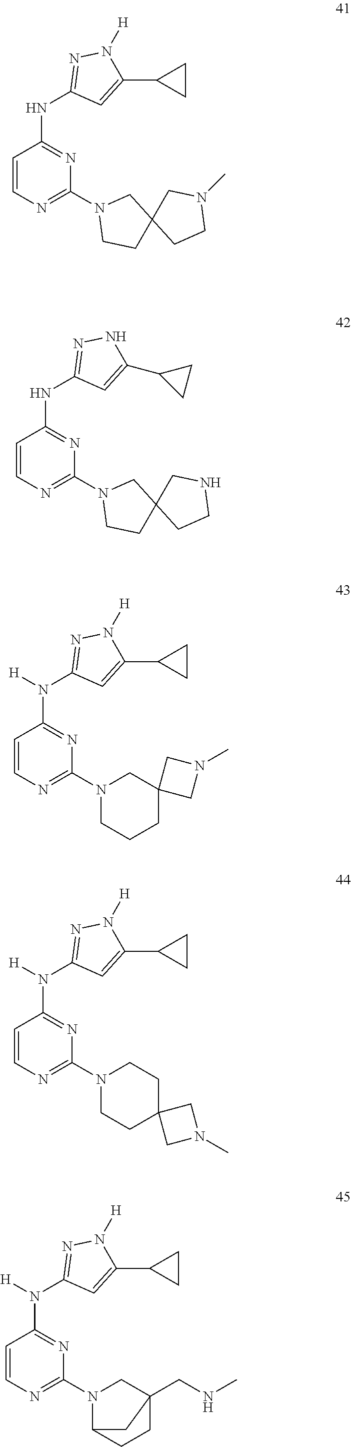 [SMILES: CN1CCC2(CCN(c3nccc(Nc4cc(C5CC5)[nH]n4)n3)C2)C1 ; c1cc(Nc2cc(C3CC3)[nH]n2)nc(N2CCC3(CCNC3)C2)n1 ; CN1CC2(CCCN(c3nccc(Nc4cc(C5CC5)[nH]n4)n3)C2)C1 ; CN1CC2(CCN(c3nccc(Nc4cc(C5CC5)[nH]n4)n3)CC2)C1 ; CNCC12CCC(C1)N(c1nccc(Nc3cc(C4CC4)[nH]n3)n1)C2]

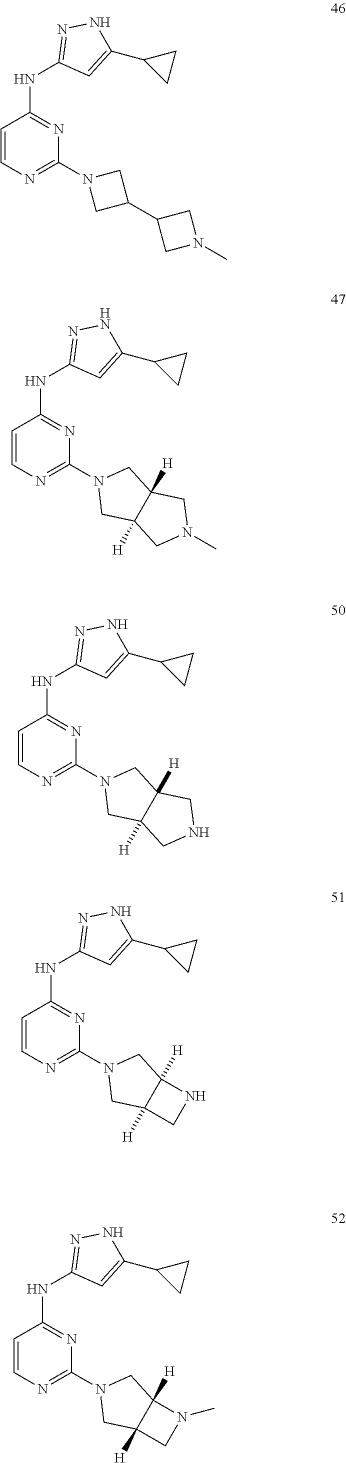 [SMILES: CN1CC(C2CN(c3nccc(Nc4cc(C5CC5)[nH]n4)n3)C2)C1 ; CN1C[C@H]2CN(c3nccc(Nc4cc(C5CC5)[nH]n4)n3)C[C@@H]2C1 ; c1cc(Nc2cc(C3CC3)[nH]n2)nc(N2C[C@@H]3CNC[C@H]3C2)n1 ; c1cc(Nc2cc(C3CC3)[nH]n2)nc(N2C[C@@H]3CN[C@@H]3C2)n1 ; CN1C[C@@H]2CN(c3nccc(Nc4cc(C5CC5)[nH]n4)n3)C[C@@H]21]

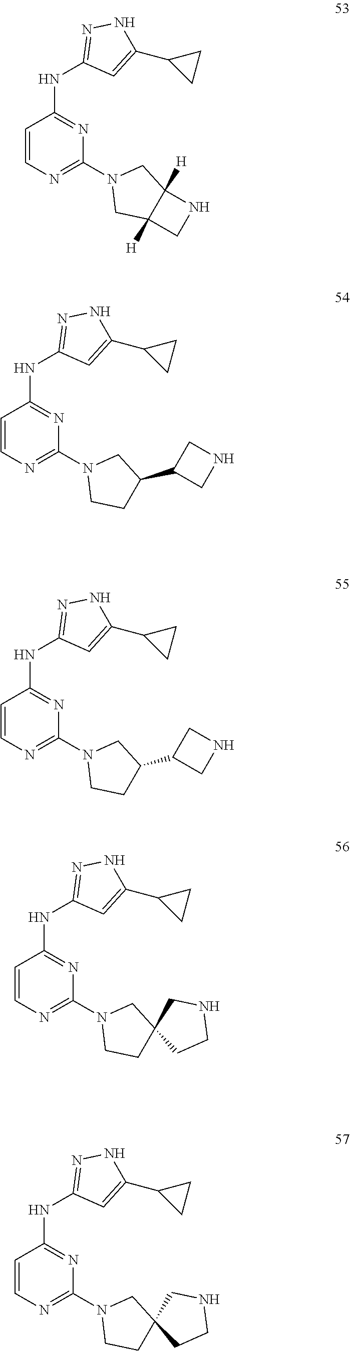 [SMILES: c1cc(Nc2cc(C3CC3)[nH]n2)nc(N2C[C@H]3CN[C@H]3C2)n1 ; c1cc(Nc2cc(C3CC3)[nH]n2)nc(N2CC[C@H](C3CNC3)C2)n1 ; c1cc(Nc2cc(C3CC3)[nH]n2)nc(N2CC[C@@H](C3CNC3)C2)n1 ; c1cc(Nc2cc(C3CC3)[nH]n2)nc(N2CC[C@@]3(CCNC3)C2)n1 ; c1cc(Nc2cc(C3CC3)[nH]n2)nc(N2CC[C@]3(CCNC3)C2)n1]

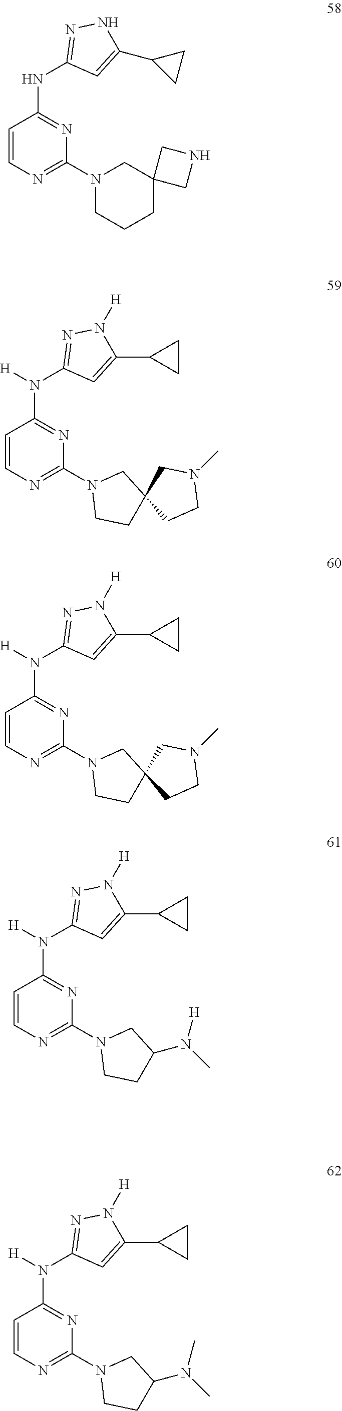 [SMILES: c1cc(Nc2cc(C3CC3)[nH]n2)nc(N2CCCC3(CNC3)C2)n1 ; CN1CC[C@@]2(CCN(c3nccc(Nc4cc(C5CC5)[nH]n4)n3)C2)C1 ; CN1CC[C@]2(CCN(c3nccc(Nc4cc(C5CC5)[nH]n4)n3)C2)C1 ; CNC1CCN(c2nccc(Nc3cc(C4CC4)[nH]n3)n2)C1 ; CN(C)C1CCN(c2nccc(Nc3cc(C4CC4)[nH]n3)n2)C1]

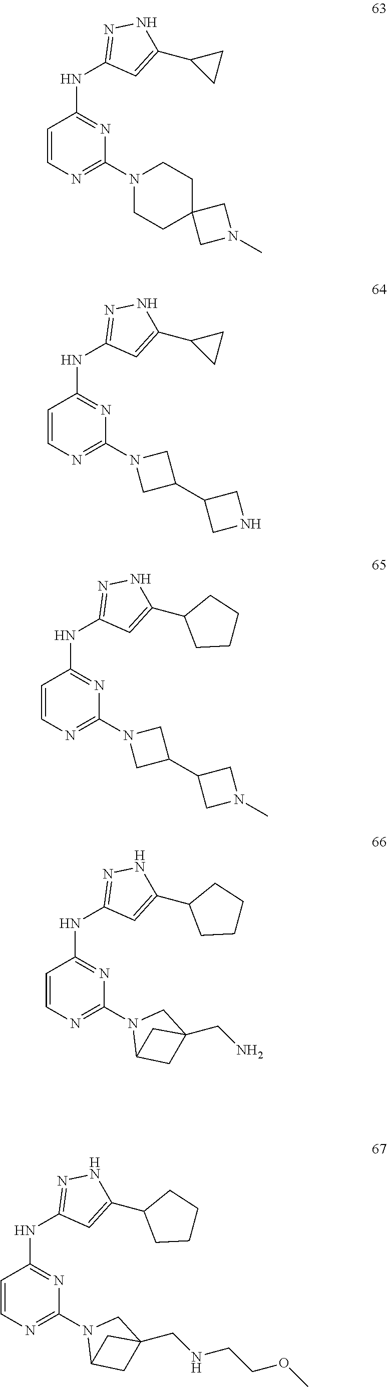 [SMILES: CN1CC2(CCN(c3nccc(Nc4cc(C5CC5)[nH]n4)n3)CC2)C1 ; c1cc(Nc2cc(C3CC3)[nH]n2)nc(N2CC(C3CNC3)C2)n1 ; CN1CC(C2CN(c3nccc(Nc4cc(C5CCCC5)[nH]n4)n3)C2)C1 ; NCC12CC(C1)N(c1nccc(Nc3cc(C4CCCC4)[nH]n3)n1)C2 ; COCCNCC12CC(C1)N(c1nccc(Nc3cc(C4CCCC4)[nH]n3)n1)C2]

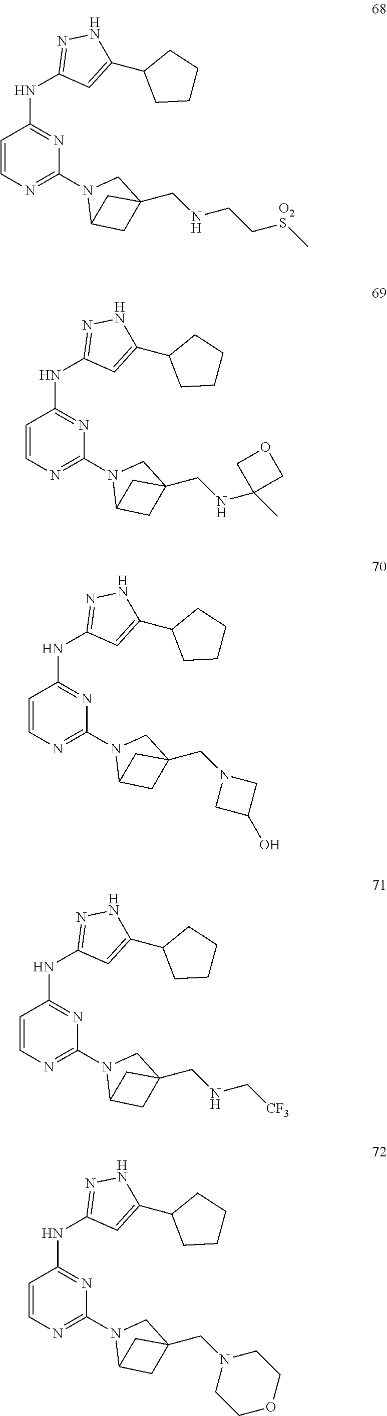 [SMILES: CS(=O)(=O)CCNCC12CC(C1)N(c1nccc(Nc3cc(C4CCCC4)[nH]n3)n1)C2 ; CC1(NCC23CC(C2)N(c2nccc(Nc4cc(C5CCCC5)[nH]n4)n2)C3)COC1 ; OC1CN(CC23CC(C2)N(c2nccc(Nc4cc(C5CCCC5)[nH]n4)n2)C3)C1 ; FC(F)(F)CNCC12CC(C1)N(c1nccc(Nc3cc(C4CCCC4)[nH]n3)n1)C2 ; c1cc(Nc2cc(C3CCCC3)[nH]n2)nc(N2CC3(CN4CCOCC4)CC2C3)n1]

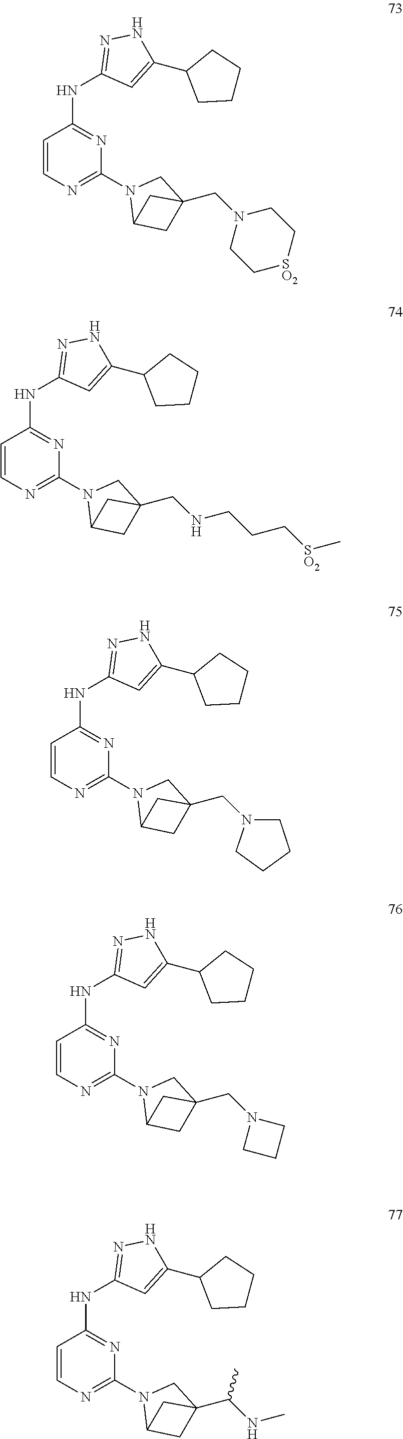 [SMILES: O=S1(=O)CCN(CC23CC(C2)N(c2nccc(Nc4cc(C5CCCC5)[nH]n4)n2)C3)CC1 ; CS(=O)(=O)CCCNCC12CC(C1)N(c1nccc(Nc3cc(C4CCCC4)[nH]n3)n1)C2 ; c1cc(Nc2cc(C3CCCC3)[nH]n2)nc(N2CC3(CN4CCCC4)CC2C3)n1 ; c1cc(Nc2cc(C3CCCC3)[nH]n2)nc(N2CC3(CN4CCC4)CC2C3)n1 ; CNC(C)C12CC(C1)N(c1nccc(Nc3cc(C4CCCC4)[nH]n3)n1)C2]

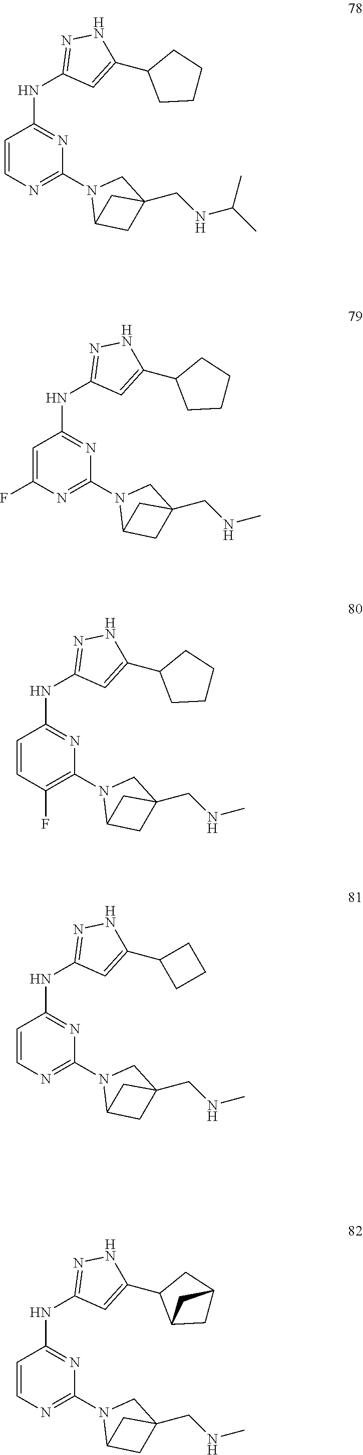 [SMILES: CC(C)NCC12CC(C1)N(c1nccc(Nc3cc(C4CCCC4)[nH]n3)n1)C2 ; CNCC12CC(C1)N(c1nc(F)cc(Nc3cc(C4CCCC4)[nH]n3)n1)C2 ; CNCC12CC(C1)N(c1nc(Nc3cc(C4CCCC4)[nH]n3)ccc1F)C2 ; CNCC12CC(C1)N(c1nccc(Nc3cc(C4CCC4)[nH]n3)n1)C2 ; CNCC12CC(C1)N(c1nccc(Nc3cc(C4C[C@H]5C[C@@H]4C5)[nH]n3)n1)C2]

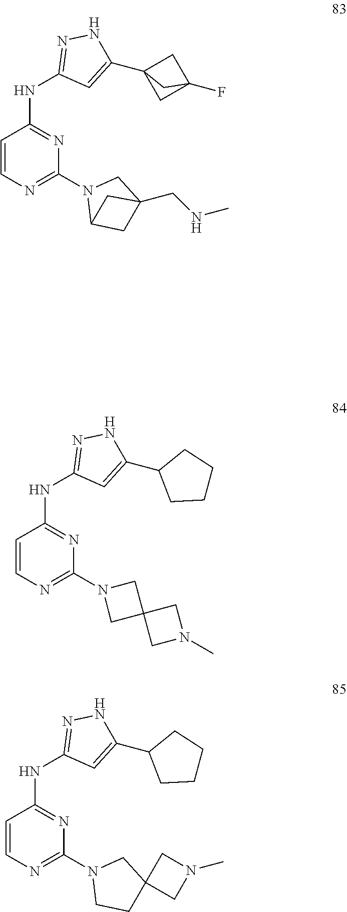 [SMILES: CNCC12CC(C1)N(c1nccc(Nc3cc(C45CC(F)(C4)C5)[nH]n3)n1)C2 ; CN1CC2(C1)CN(c1nccc(Nc3cc(C4CCCC4)[nH]n3)n1)C2 ; CN1CC2(CCN(c3nccc(Nc4cc(C5CCCC5)[nH]n4)n3)C2)C1]

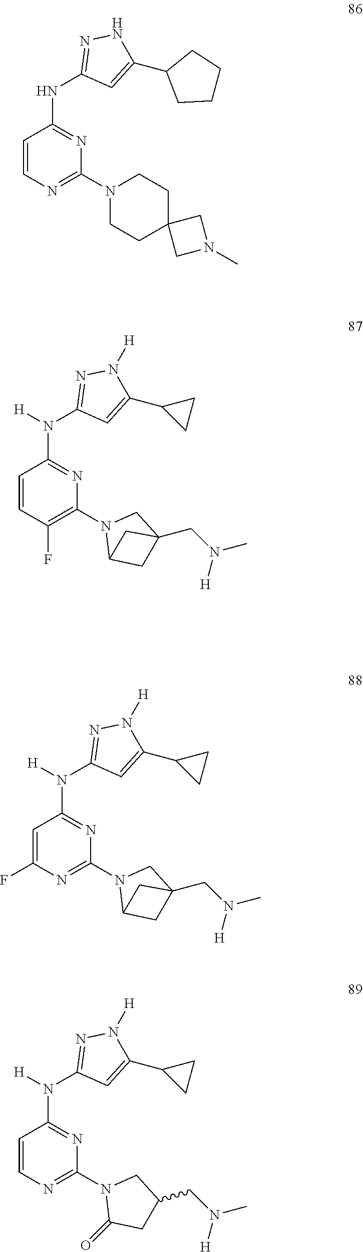 [SMILES: CN1CC2(CCN(c3nccc(Nc4cc(C5CCCC5)[nH]n4)n3)CC2)C1 ; CNCC12CC(C1)N(c1nc(Nc3cc(C4CC4)[nH]n3)ccc1F)C2 ; CNCC12CC(C1)N(c1nc(F)cc(Nc3cc(C4CC4)[nH]n3)n1)C2 ; CNCC1CC(=O)N(c2nccc(Nc3cc(C4CC4)[nH]n3)n2)C1]

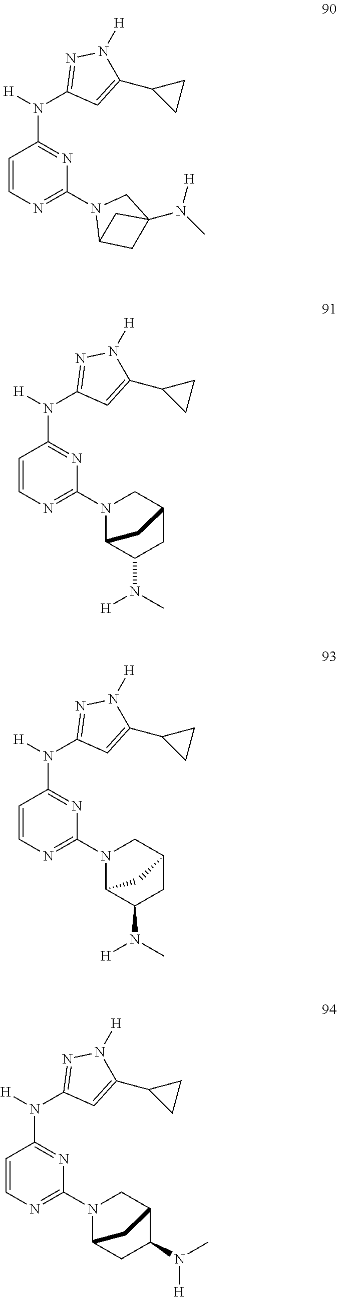

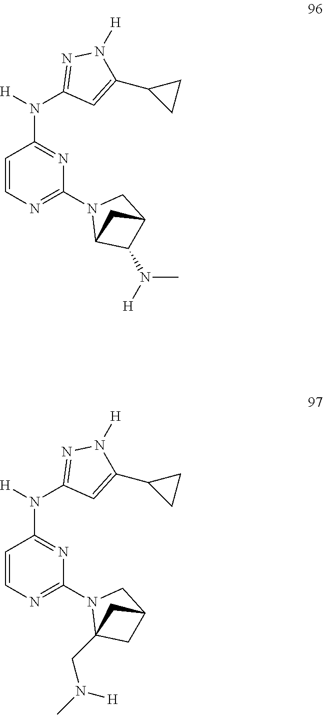 [SMILES: CN[C@H]1[C@@H]2C[C@H]1N(c1nccc(Nc3cc(C4CC4)[nH]n3)n1)C2 ; CNC[C@]12C[C@H](CN1c1nccc(Nc3cc(C4CC4)[nH]n3)n1)C2]

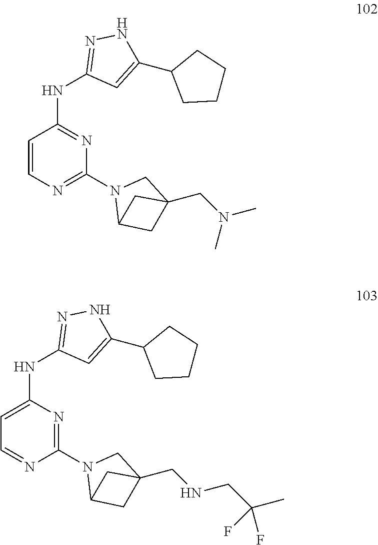 [SMILES: CN(C)CC12CC(C1)N(c1nccc(Nc3cc(C4CCCC4)[nH]n3)n1)C2 ; CC(F)(F)CNCC12CC(C1)N(c1nccc(Nc3cc(C4CCCC4)[nH]n3)n1)C2]

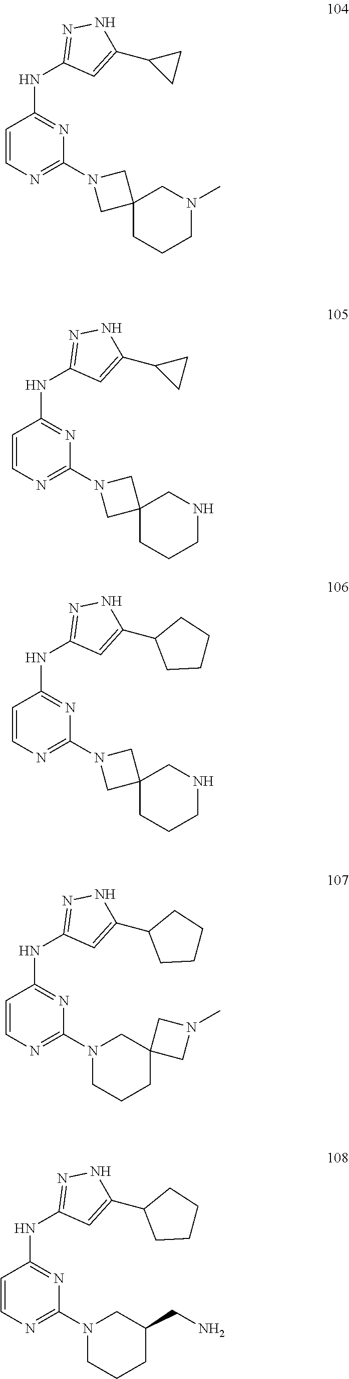 [SMILES: CN1CCCC2(C1)CN(c1nccc(Nc3cc(C4CC4)[nH]n3)n1)C2 ; c1cc(Nc2cc(C3CC3)[nH]n2)nc(N2CC3(CCCNC3)C2)n1 ; c1cc(Nc2cc(C3CCCC3)[nH]n2)nc(N2CC3(CCCNC3)C2)n1 ; CN1CC2(CCCN(c3nccc(Nc4cc(C5CCCC5)[nH]n4)n3)C2)C1 ; NC[C@H]1CCCN(c2nccc(Nc3cc(C4CCCC4)[nH]n3)n2)C1]

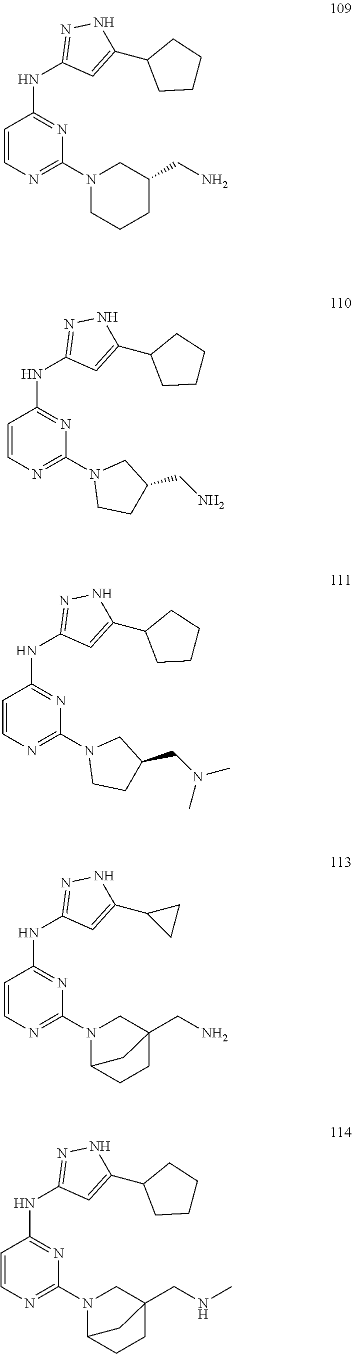 [SMILES: NC[C@@H]1CCCN(c2nccc(Nc3cc(C4CCCC4)[nH]n3)n2)C1 ; NC[C@@H]1CCN(c2nccc(Nc3cc(C4CCCC4)[nH]n3)n2)C1 ; CN(C)C[C@H]1CCN(c2nccc(Nc3cc(C4CCCC4)[nH]n3)n2)C1 ; NCC12CCC(C1)N(c1nccc(Nc3cc(C4CC4)[nH]n3)n1)C2 ; CNCC12CCC(C1)N(c1nccc(Nc3cc(C4CCCC4)[nH]n3)n1)C2]

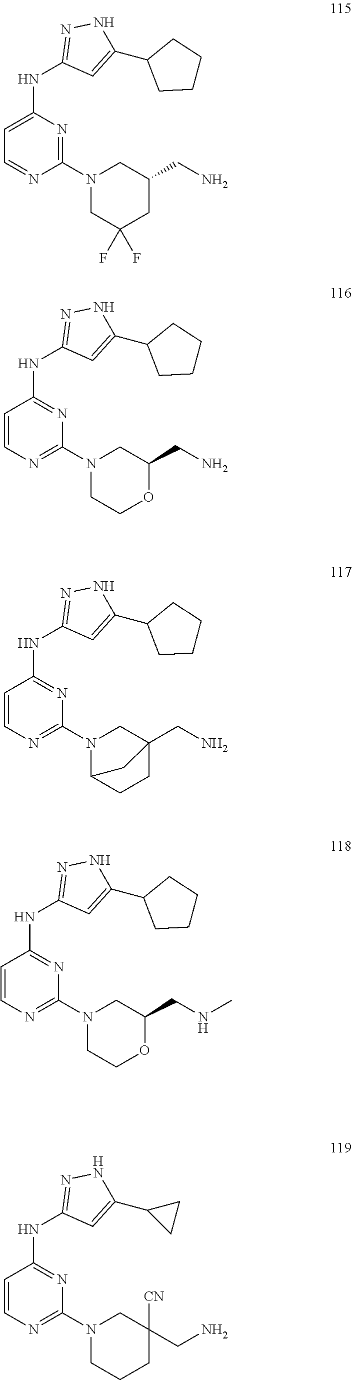 [SMILES: NC[C@H]1CN(c2nccc(Nc3cc(C4CCCC4)[nH]n3)n2)CC(F)(F)C1 ; NC[C@@H]1CN(c2nccc(Nc3cc(C4CCCC4)[nH]n3)n2)CCO1 ; NCC12CCC(C1)N(c1nccc(Nc3cc(C4CCCC4)[nH]n3)n1)C2 ; CNC[C@@H]1CN(c2nccc(Nc3cc(C4CCCC4)[nH]n3)n2)CCO1 ; N#CC1(CN)CCCN(c2nccc(Nc3cc(C4CC4)[nH]n3)n2)C1]

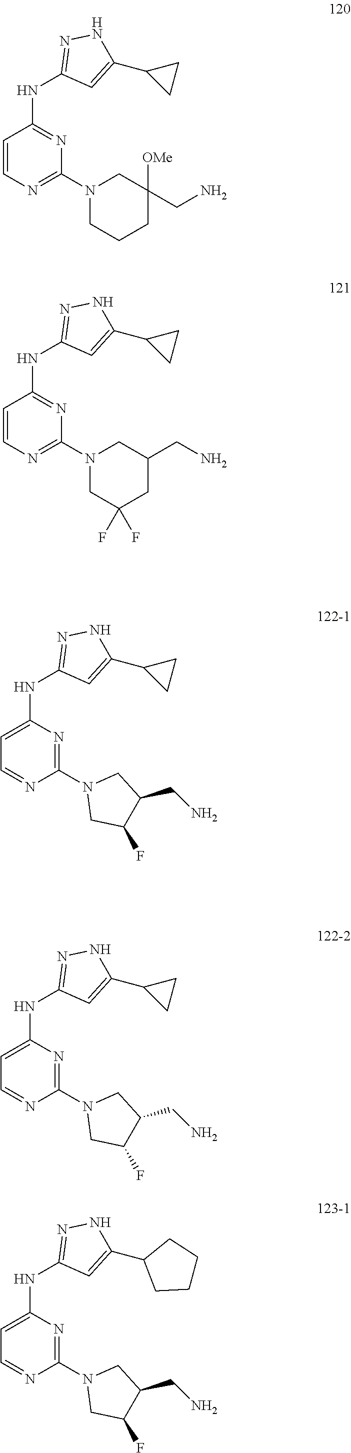 [SMILES: COC1(CN)CCCN(c2nccc(Nc3cc(C4CC4)[nH]n3)n2)C1 ; NCC1CN(c2nccc(Nc3cc(C4CC4)[nH]n3)n2)CC(F)(F)C1 ; NC[C@@H]1CN(c2nccc(Nc3cc(C4CC4)[nH]n3)n2)C[C@@H]1F ; NC[C@H]1CN(c2nccc(Nc3cc(C4CC4)[nH]n3)n2)C[C@H]1F ; NC[C@@H]1CN(c2nccc(Nc3cc(C4CCCC4)[nH]n3)n2)C[C@@H]1F]

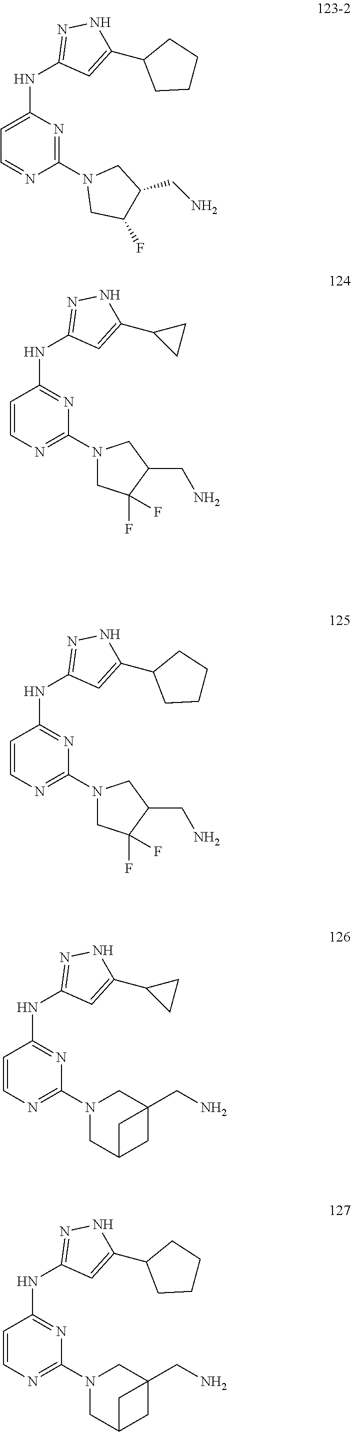 [SMILES: NC[C@H]1CN(c2nccc(Nc3cc(C4CCCC4)[nH]n3)n2)C[C@H]1F ; NCC1CN(c2nccc(Nc3cc(C4CC4)[nH]n3)n2)CC1(F)F ; NCC1CN(c2nccc(Nc3cc(C4CCCC4)[nH]n3)n2)CC1(F)F ; NCC12CC(CN(c3nccc(Nc4cc(C5CC5)[nH]n4)n3)C1)C2 ; NCC12CC(CN(c3nccc(Nc4cc(C5CCCC5)[nH]n4)n3)C1)C2]

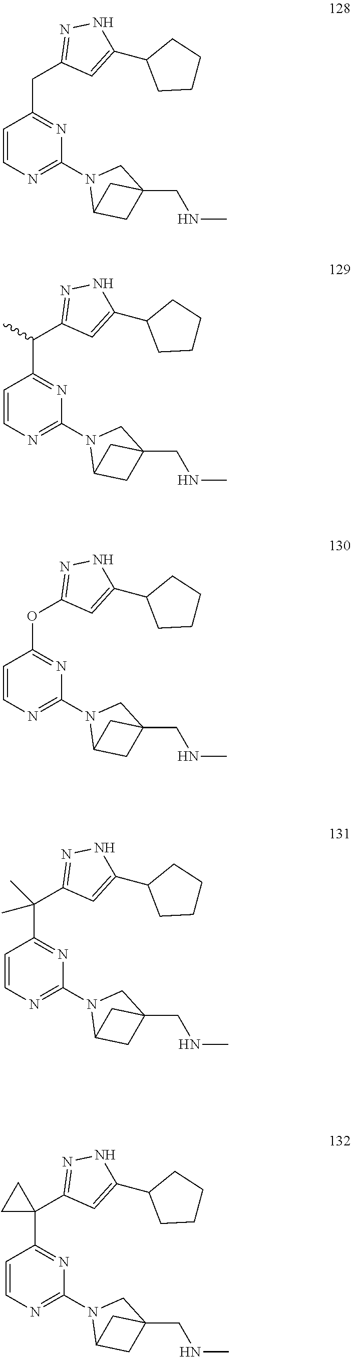

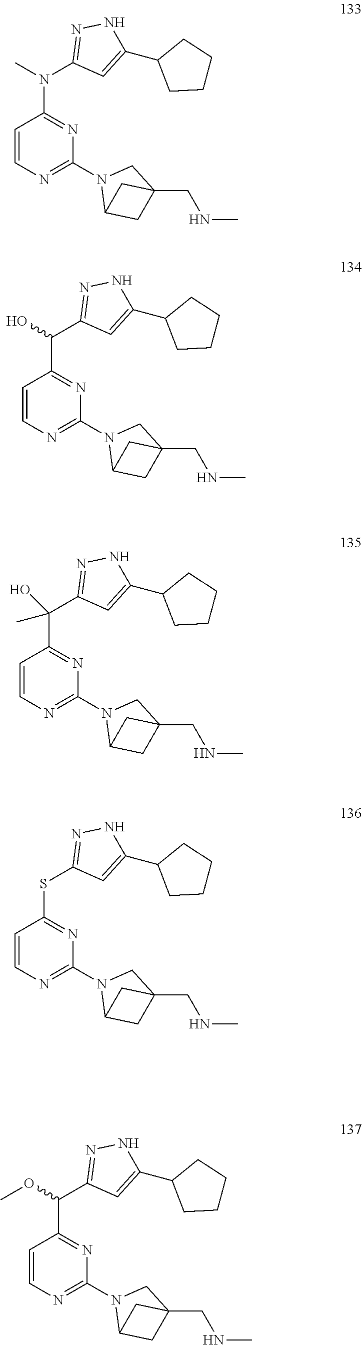

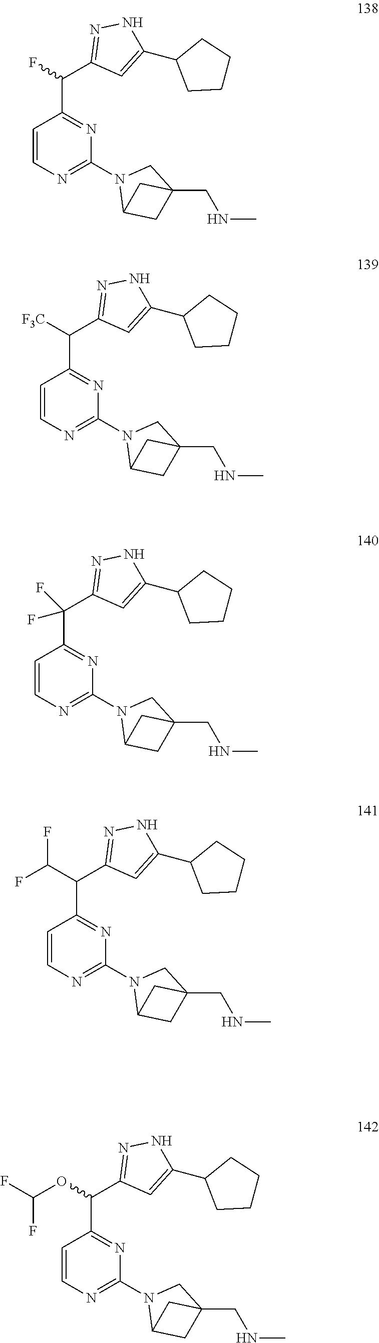 [SMILES: CNCC12CC(C1)N(c1nccc(C(F)c3cc(C4CCCC4)[nH]n3)n1)C2 ; CNCC12CC(C1)N(c1nccc(C(c3cc(C4CCCC4)[nH]n3)C(F)(F)F)n1)C2 ; CNCC12CC(C1)N(c1nccc(C(F)(F)c3cc(C4CCCC4)[nH]n3)n1)C2 ; CNCC12CC(C1)N(c1nccc(C(c3cc(C4CCCC4)[nH]n3)C(F)F)n1)C2 ; CNCC12CC(C1)N(c1nccc(C(OC(F)F)c3cc(C4CCCC4)[nH]n3)n1)C2]

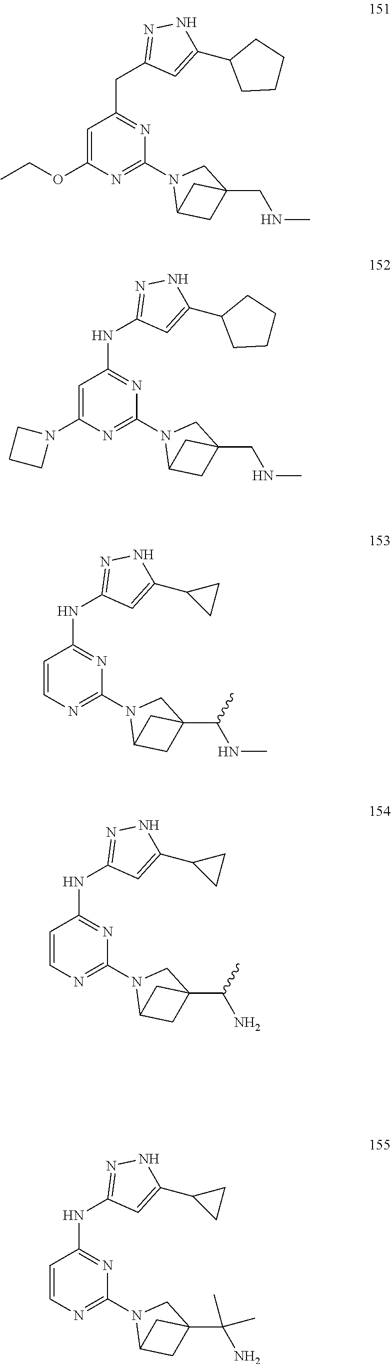

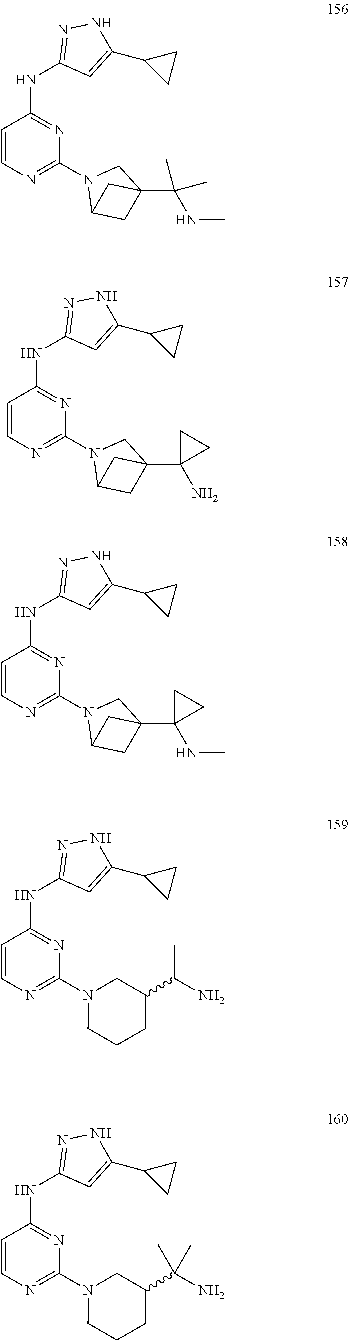 [SMILES: CNC(C)(C)C12CC(C1)N(c1nccc(Nc3cc(C4CC4)[nH]n3)n1)C2 ; NC1(C23CC(C2)N(c2nccc(Nc4cc(C5CC5)[nH]n4)n2)C3)CC1 ; CNC1(C23CC(C2)N(c2nccc(Nc4cc(C5CC5)[nH]n4)n2)C3)CC1 ; CC(N)C1CCCN(c2nccc(Nc3cc(C4CC4)[nH]n3)n2)C1 ; CC(C)(N)C1CCCN(c2nccc(Nc3cc(C4CC4)[nH]n3)n2)C1]

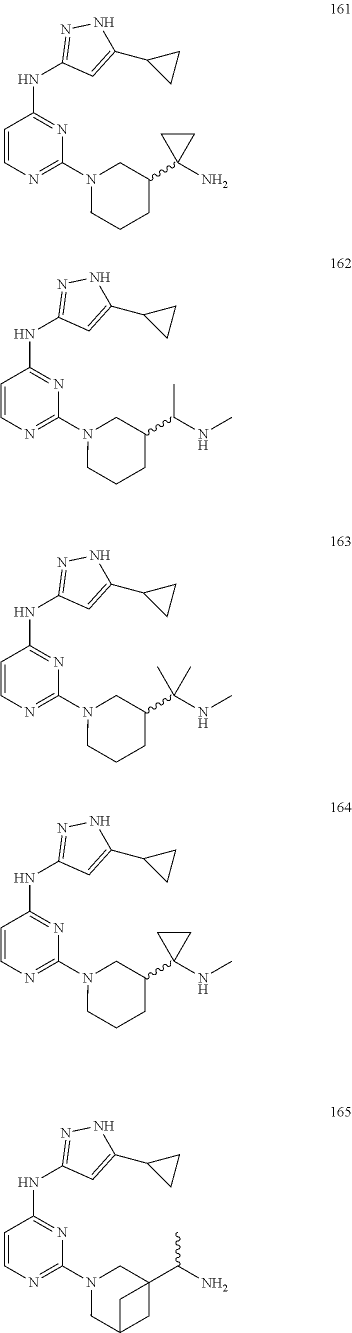 [SMILES: NC1(C2CCCN(c3nccc(Nc4cc(C5CC5)[nH]n4)n3)C2)CC1 ; CNC(C)C1CCCN(c2nccc(Nc3cc(C4CC4)[nH]n3)n2)C1 ; CNC(C)(C)C1CCCN(c2nccc(Nc3cc(C4CC4)[nH]n3)n2)C1 ; CNC1(C2CCCN(c3nccc(Nc4cc(C5CC5)[nH]n4)n3)C2)CC1 ; CC(N)C12CC(CN(c3nccc(Nc4cc(C5CC5)[nH]n4)n3)C1)C2]

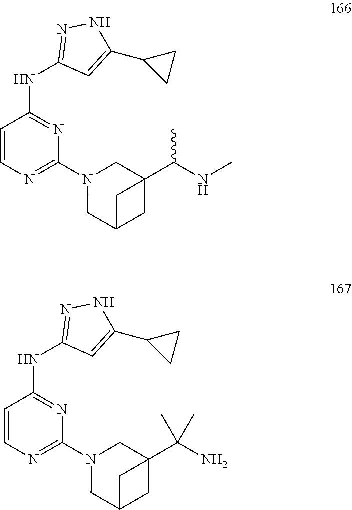 [SMILES: CNC(C)C12CC(CN(c3nccc(Nc4cc(C5CC5)[nH]n4)n3)C1)C2 ; CC(C)(N)C12CC(CN(c3nccc(Nc4cc(C5CC5)[nH]n4)n3)C1)C2]

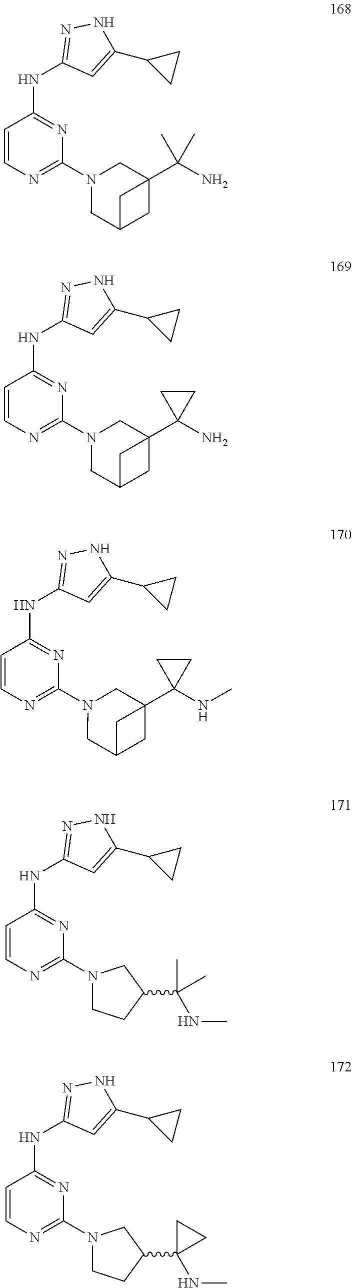 [SMILES: CC(C)(N)C12CC(CN(c3nccc(Nc4cc(C5CC5)[nH]n4)n3)C1)C2 ; NC1(C23CC(CN(c4nccc(Nc5cc(C6CC6)[nH]n5)n4)C2)C3)CC1 ; CNC1(C23CC(CN(c4nccc(Nc5cc(C6CC6)[nH]n5)n4)C2)C3)CC1 ; CNC(C)(C)C1CCN(c2nccc(Nc3cc(C4CC4)[nH]n3)n2)C1 ; CNC1(C2CCN(c3nccc(Nc4cc(C5CC5)[nH]n4)n3)C2)CC1]

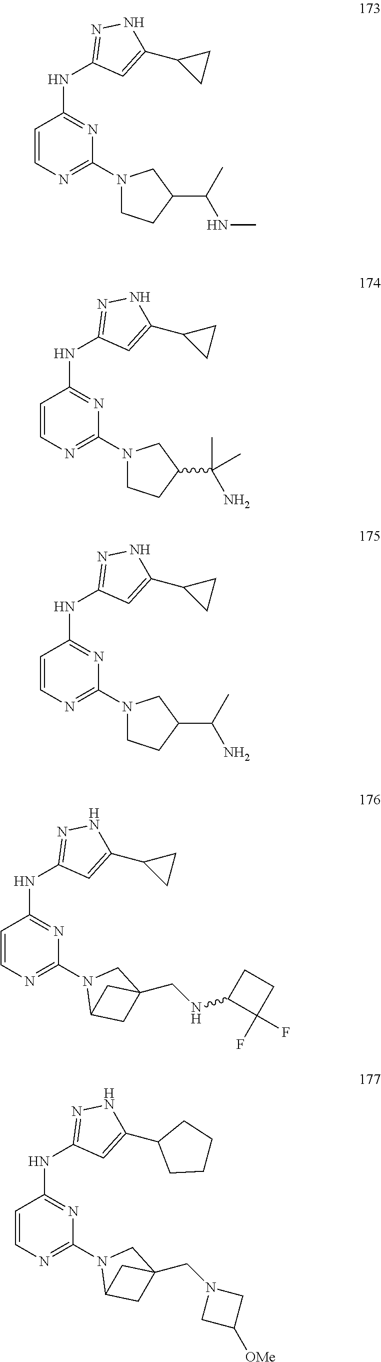 [SMILES: CNC(C)C1CCN(c2nccc(Nc3cc(C4CC4)[nH]n3)n2)C1 ; CC(C)(N)C1CCN(c2nccc(Nc3cc(C4CC4)[nH]n3)n2)C1 ; CC(N)C1CCN(c2nccc(Nc3cc(C4CC4)[nH]n3)n2)C1 ; FC1(F)CCC1NCC12CC(C1)N(c1nccc(Nc3cc(C4CC4)[nH]n3)n1)C2 ; COC1CN(CC23CC(C2)N(c2nccc(Nc4cc(C5CCCC5)[nH]n4)n2)C3)C1]

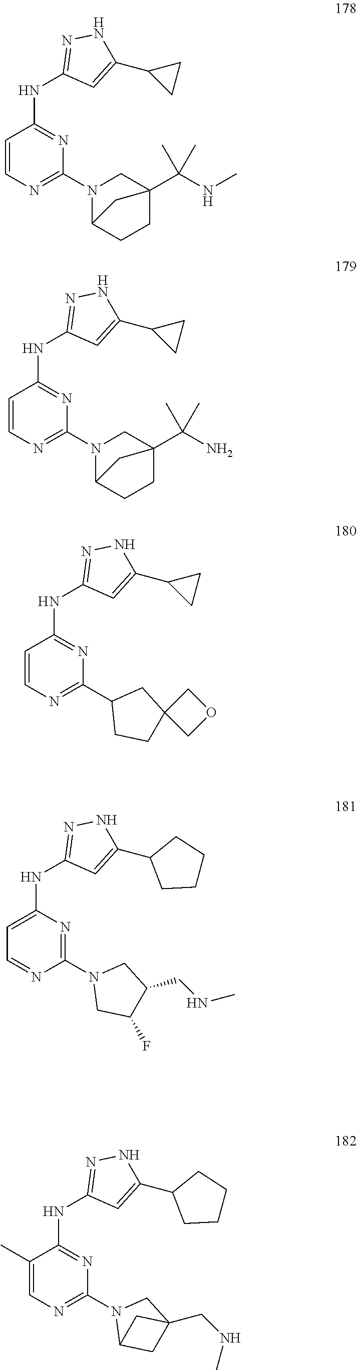

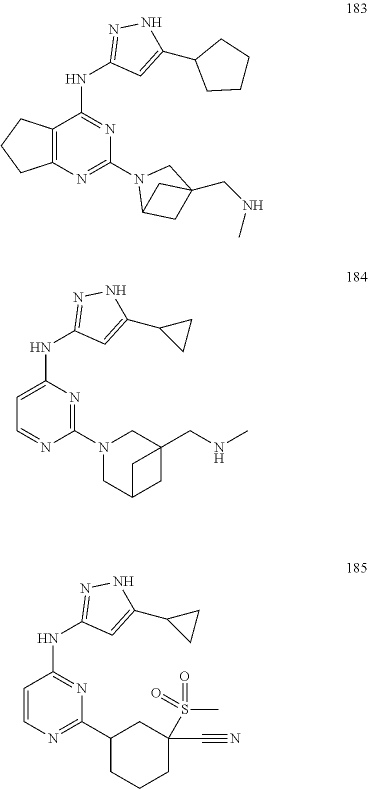 [SMILES: CNCC12CC(C1)N(c1nc3c(c(Nc4cc(C5CCCC5)[nH]n4)n1)CCC3)C2 ; CNCC12CC(CN(c3nccc(Nc4cc(C5CC5)[nH]n4)n3)C1)C2 ; CS(=O)(=O)C1(C#N)CCCC(c2nccc(Nc3cc(C4CC4)[nH]n3)n2)C1]

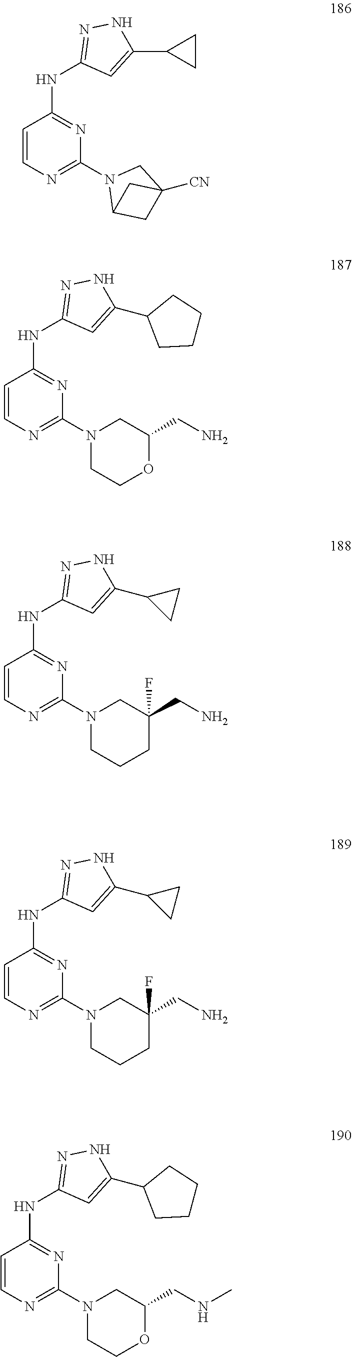 [SMILES: N#CC12CC(C1)N(c1nccc(Nc3cc(C4CC4)[nH]n3)n1)C2 ; NC[C@H]1CN(c2nccc(Nc3cc(C4CCCC4)[nH]n3)n2)CCO1 ; NC[C@@]1(F)CCCN(c2nccc(Nc3cc(C4CC4)[nH]n3)n2)C1 ; NC[C@]1(F)CCCN(c2nccc(Nc3cc(C4CC4)[nH]n3)n2)C1 ; CNC[C@H]1CN(c2nccc(Nc3cc(C4CCCC4)[nH]n3)n2)CCO1]

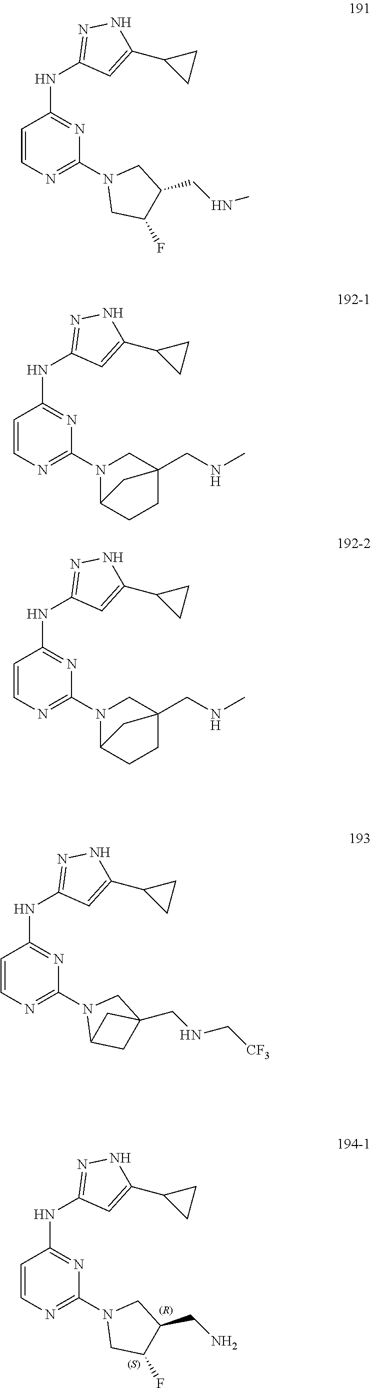

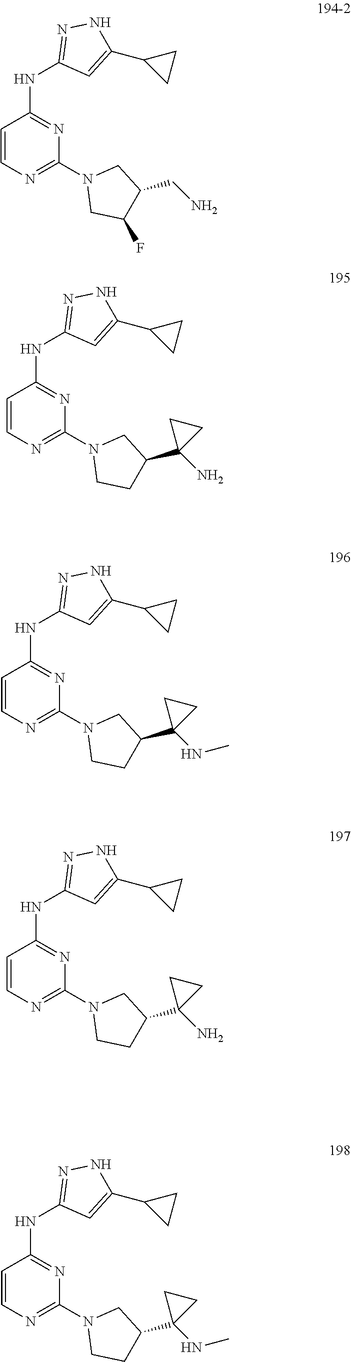 [SMILES: NC[C@H]1CN(c2nccc(Nc3cc(C4CC4)[nH]n3)n2)C[C@@H]1F ; NC1([C@H]2CCN(c3nccc(Nc4cc(C5CC5)[nH]n4)n3)C2)CC1 ; CNC1([C@H]2CCN(c3nccc(Nc4cc(C5CC5)[nH]n4)n3)C2)CC1 ; NC1([C@@H]2CCN(c3nccc(Nc4cc(C5CC5)[nH]n4)n3)C2)CC1 ; CNC1([C@@H]2CCN(c3nccc(Nc4cc(C5CC5)[nH]n4)n3)C2)CC1]

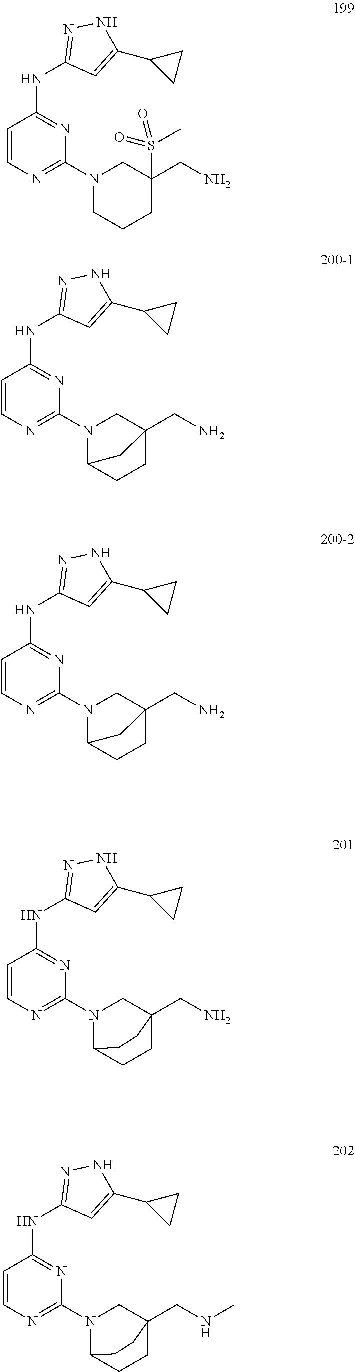 [SMILES: CS(=O)(=O)C1(CN)CCCN(c2nccc(Nc3cc(C4CC4)[nH]n3)n2)C1 ; NCC12CCC(C1)N(c1nccc(Nc3cc(C4CC4)[nH]n3)n1)C2 ; NCC12CCC(C1)N(c1nccc(Nc3cc(C4CC4)[nH]n3)n1)C2 ; NCC12CCC(CC1)N(c1nccc(Nc3cc(C4CC4)[nH]n3)n1)C2 ; CNCC12CCC(CC1)N(c1nccc(Nc3cc(C4CC4)[nH]n3)n1)C2]

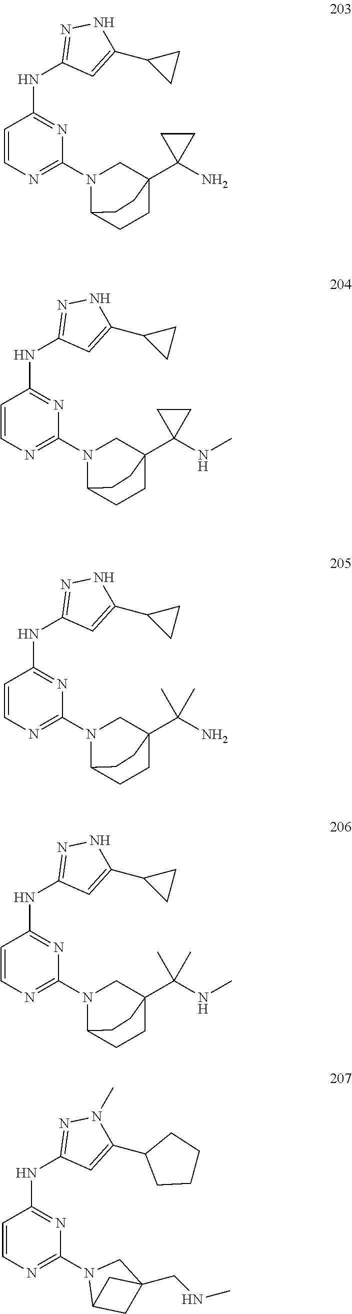

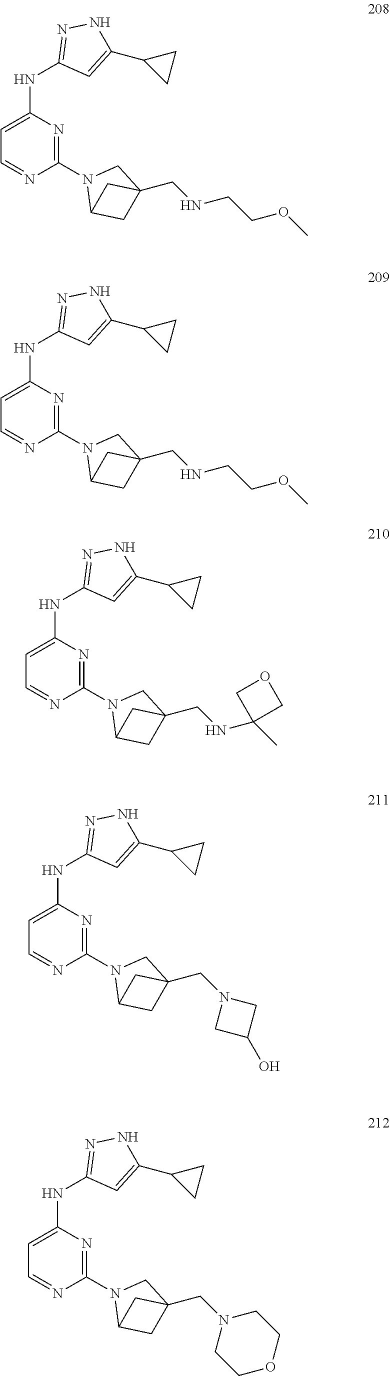 [SMILES: COCCNCC12CC(C1)N(c1nccc(Nc3cc(C4CC4)[nH]n3)n1)C2 ; COCCNCC12CC(C1)N(c1nccc(Nc3cc(C4CC4)[nH]n3)n1)C2 ; CC1(NCC23CC(C2)N(c2nccc(Nc4cc(C5CC5)[nH]n4)n2)C3)COC1 ; OC1CN(CC23CC(C2)N(c2nccc(Nc4cc(C5CC5)[nH]n4)n2)C3)C1 ; c1cc(Nc2cc(C3CC3)[nH]n2)nc(N2CC3(CN4CCOCC4)CC2C3)n1]

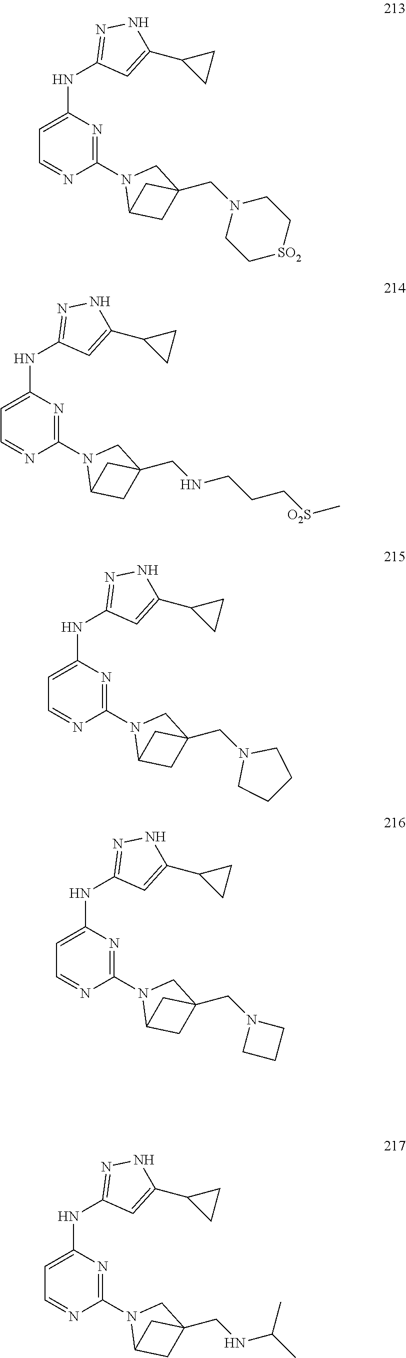 [SMILES: O=S1(=O)CCN(CC23CC(C2)N(c2nccc(Nc4cc(C5CC5)[nH]n4)n2)C3)CC1 ; CS(=O)(=O)CCCNCC12CC(C1)N(c1nccc(Nc3cc(C4CC4)[nH]n3)n1)C2 ; c1cc(Nc2cc(C3CC3)[nH]n2)nc(N2CC3(CN4CCCC4)CC2C3)n1 ; c1cc(Nc2cc(C3CC3)[nH]n2)nc(N2CC3(CN4CCC4)CC2C3)n1 ; CC(C)NCC12CC(C1)N(c1nccc(Nc3cc(C4CC4)[nH]n3)n1)C2]

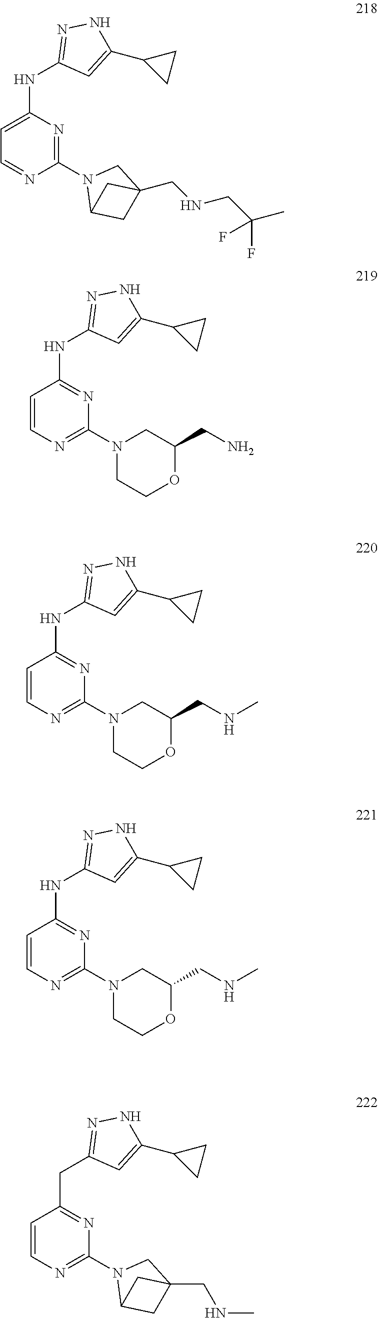 [SMILES: CC(F)(F)CNCC12CC(C1)N(c1nccc(Nc3cc(C4CC4)[nH]n3)n1)C2 ; NC[C@@H]1CN(c2nccc(Nc3cc(C4CC4)[nH]n3)n2)CCO1 ; CNC[C@@H]1CN(c2nccc(Nc3cc(C4CC4)[nH]n3)n2)CCO1 ; CNC[C@H]1CN(c2nccc(Nc3cc(C4CC4)[nH]n3)n2)CCO1 ; CNCC12CC(C1)N(c1nccc(Cc3cc(C4CC4)[nH]n3)n1)C2]

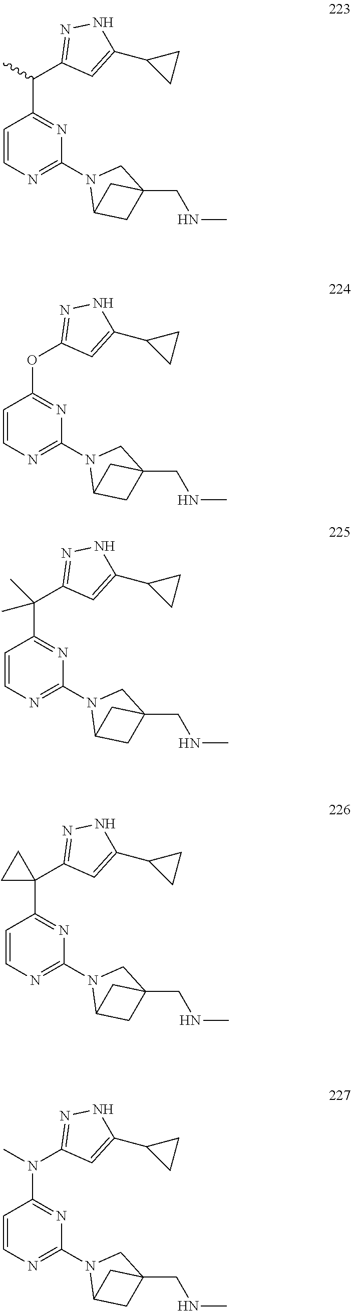

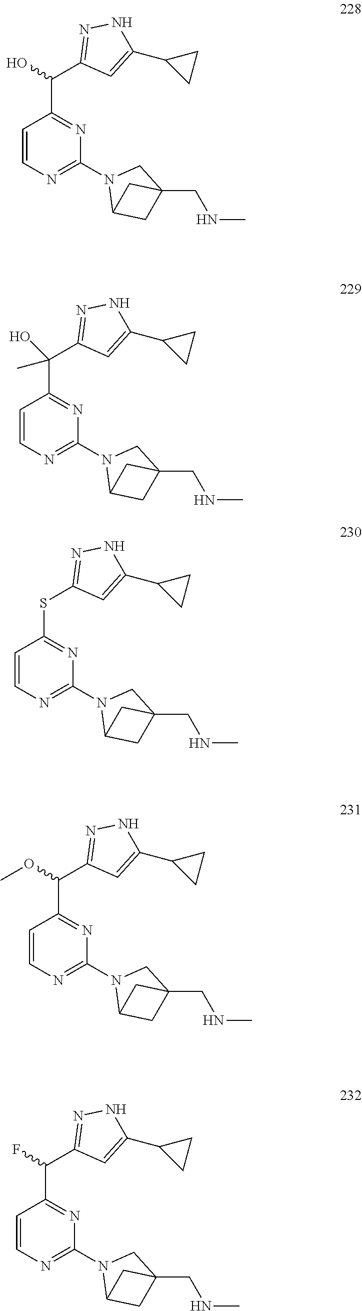

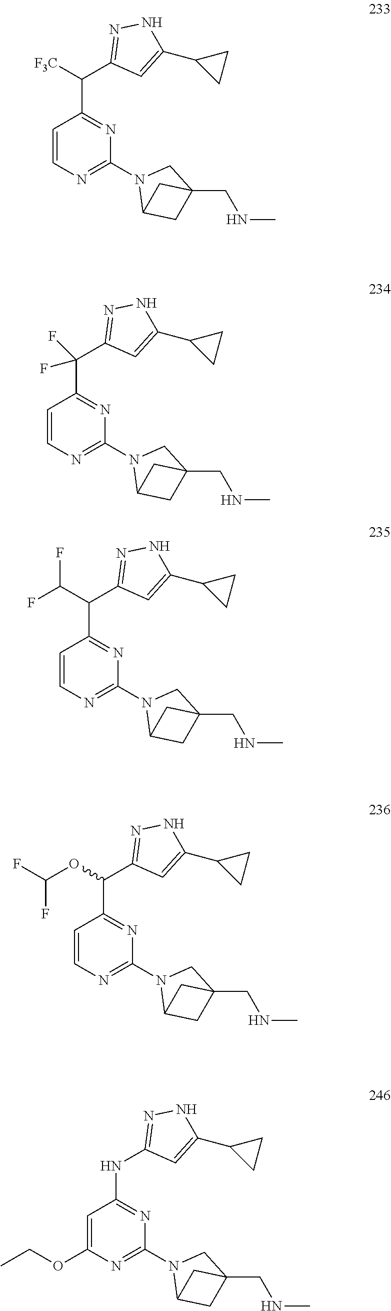

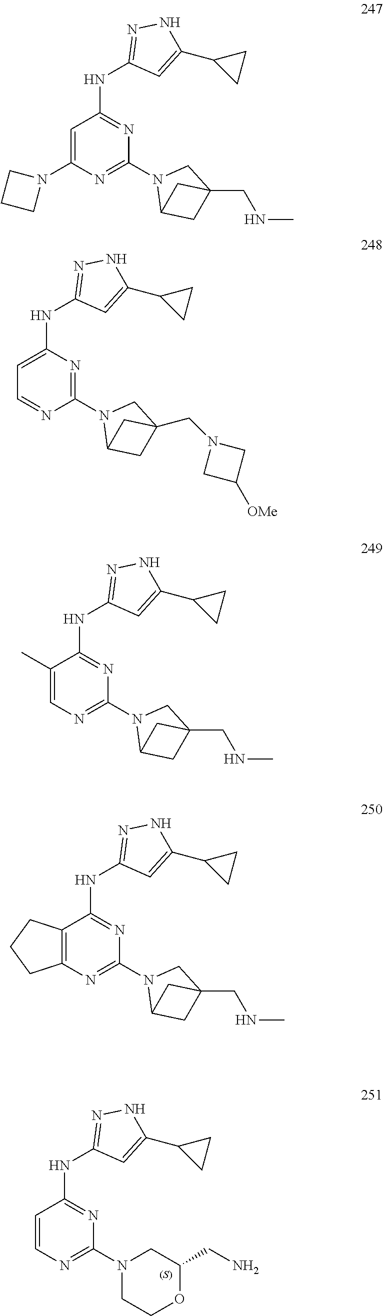

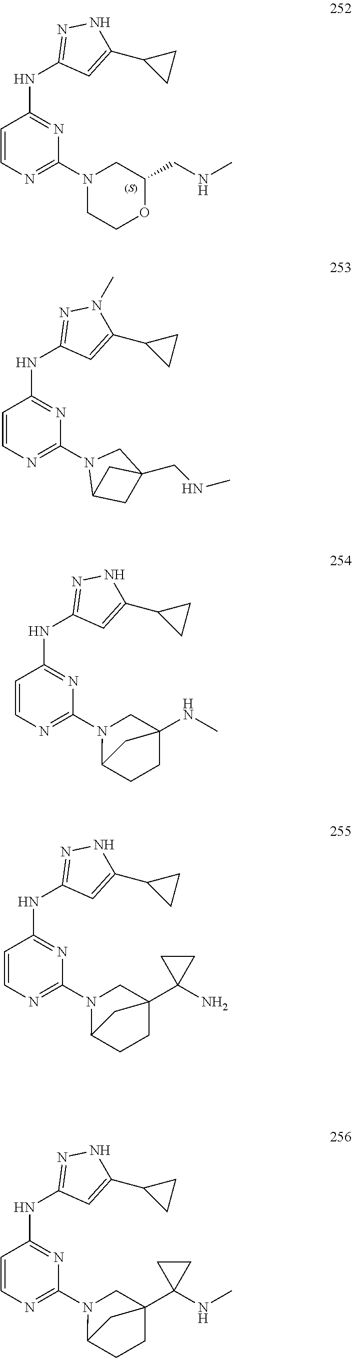

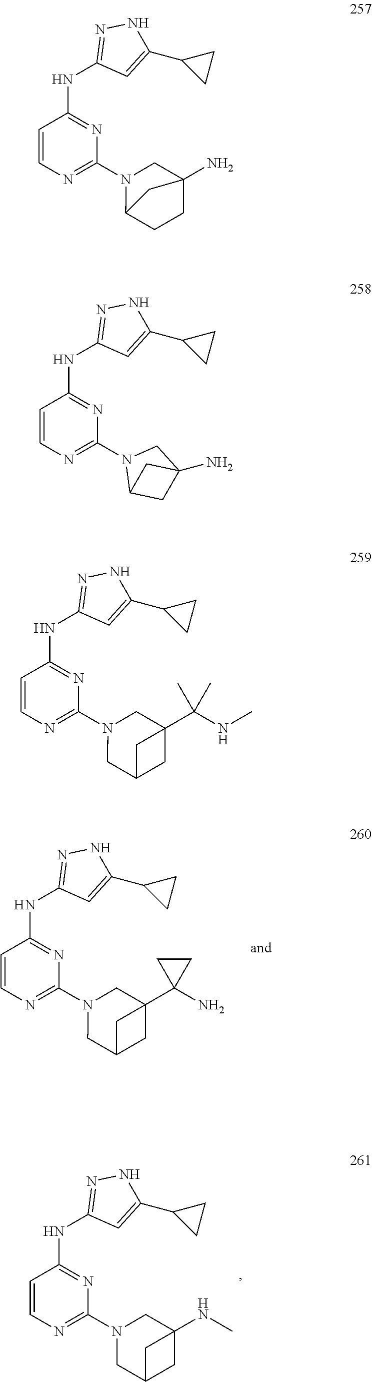 [SMILES: NC12CCC(C1)N(c1nccc(Nc3cc(C4CC4)[nH]n3)n1)C2 ; NC12CC(C1)N(c1nccc(Nc3cc(C4CC4)[nH]n3)n1)C2 ; CNC(C)(C)C12CC(CN(c3nccc(Nc4cc(C5CC5)[nH]n4)n3)C1)C2 ; NC1(C23CC(CN(c4nccc(Nc5cc(C6CC6)[nH]n5)n4)C2)C3)CC1 ; CNC12CC(CN(c3nccc(Nc4cc(C5CC5)[nH]n4)n3)C1)C2]

or a pharmaceutically acceptable salt, stereoisomer, and/or N-oxide thereof.

Also contemplated herein is a compound represented by Formula II:

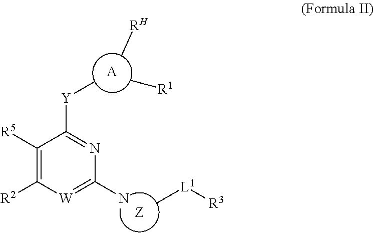

or a pharmaceutically acceptable salt, stereoisomer, and/or N-oxide thereof, wherein:
   W is selected from the group consisting of N, C—H, and C—F;
   Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
   ring A is a 5-6 membered heteroaryl optionally substituted with C₁-C₄ alkyl;
   ring Z is selected from the group consisting of 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, oxo, C₁-C₄alkoxy, and cyano;
   L¹ is bond or C₁-C₆ alkylene (optionally substituted by one, two or three R⁴);
   R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
   R⁴ is each independently selected from the group consisting of halogen and C₁-C₆ alkyl, or two R⁴ together with the carbon from which the R⁴ are attached form a C₃-C₅ carbocyclyl;
   R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
   R^{H} is selected from the group consisting of H, C₁-C₃alkyl, —C(O)—C₁-C₃alkyl, and C(O)—O—C₁-C₃alkyl;
   R^{N} is selected from the group consisting of H and C₁-C₃alkyl, or two R^{N} together with the nitrogen atoms to which the R^{N}s are attached form a 4-6 membered heterocyclic ring;
   R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
   R² is selected from the group consisting of H, F, CF₃, —C(O)—O-methyl, —C(O)OH, —O-methyl, —O-ethyl, C₃-C₇ cycloalkyl, and heterocyclyl; and
   R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring.

Exemplary contemplated compounds are represented by Formula II-A:

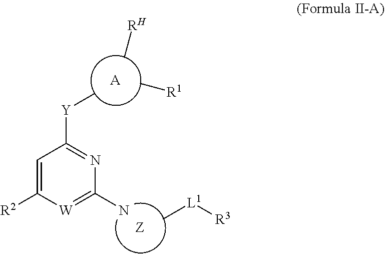

or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CF₂, CH(CH₃), C(CH₃)₂, and C(CH₂CH₂);
ring A is a 5-6 membered heteroaryl;
ring Z is selected from the group consisting of 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, and oxo;
L¹ is bond or C₁-C₆ alkylene (optionally substituted by one, two or three halogens);
R³ is selected from the group consisting of H, C₁-C₆ alkyl, NR′R′, and heterocyclyl optionally substituted by one or two substituents each independently selected from the group consisting of halo, hydroxyl, C₁-C₄ alkyl (optionally substituted by one, two or three halogens), and oxo;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, heterocyclyl, or heteroaryl may be substituted by one, two or three substituents each independently selected from halo and C₁-C₄alkyl (optionally substituted by one, two or three halogens); and
R² is selected from the group consisting of H, F, CF₃, —C(O)—O-methyl, —C(O)OH, —O-methyl, C₃-C₇ cycloalkyl, and heterocyclyl.

In some embodiments, ring A is 5-6 membered heteroaryl comprising at least one nitrogen atom in the heteroaryl. For example, ring A may be pyridyl, pyrimidyl, pyrazinyl, triazinyl, pyrazolyl, imidazolyl, or triazolyl.

In some embodiments, W is C—H. In other embodiments, W is C—F. In other embodiments, W is N.

In some embodiments, R^{H} is selected from the group consisting of H, —C(O)—C₁-C₃alkyl, and C(O)—O—C₁-C₃alkyl. In some embodiments, R^{H} is H.

In some embodiments, R¹ is a 5-6 membered heterocyclyl or C₃-C₆ cycloalkyl. In some embodiments, R¹ is a C₃-C₆ cycloalkyl. For example, R¹ may be cyclopropyl or cyclopentyl.

In some embodiments, ring Z is selected from the group consisting of 6-10 membered spiroheterocycle, a 6-10 membered fused bicyclic heterocyclic, and a 6-10 membered bridged cycloheteroalkyl, wherein Z may optionally be substituted by one or two substituents each independently selected from the group consisting of halo, hydroxyl, C₁-C₄ alkyl (optionally substituted by one, two or three halogens), and oxo.

In some embodiments, ring Z is selected from the group consisting of:

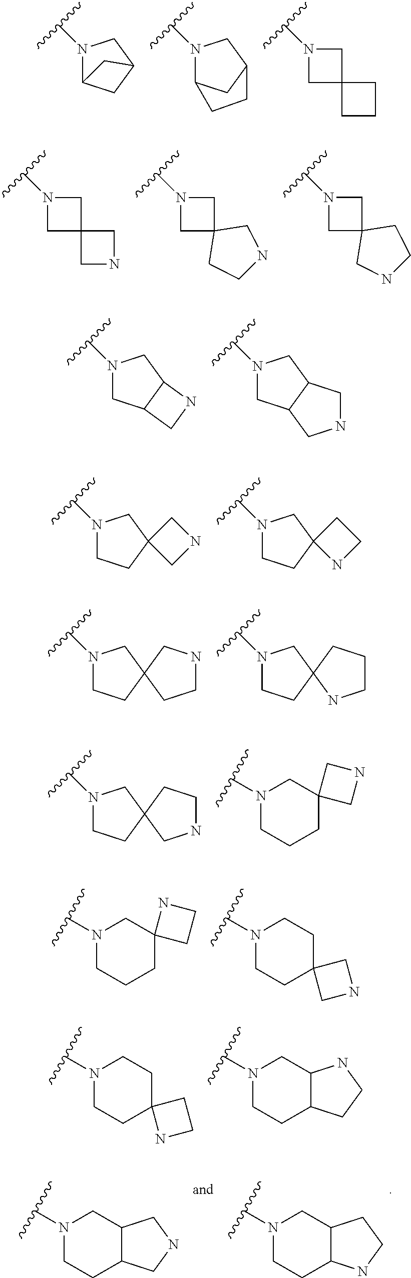

In some embodiments, ring Z is selected from the group consisting of:

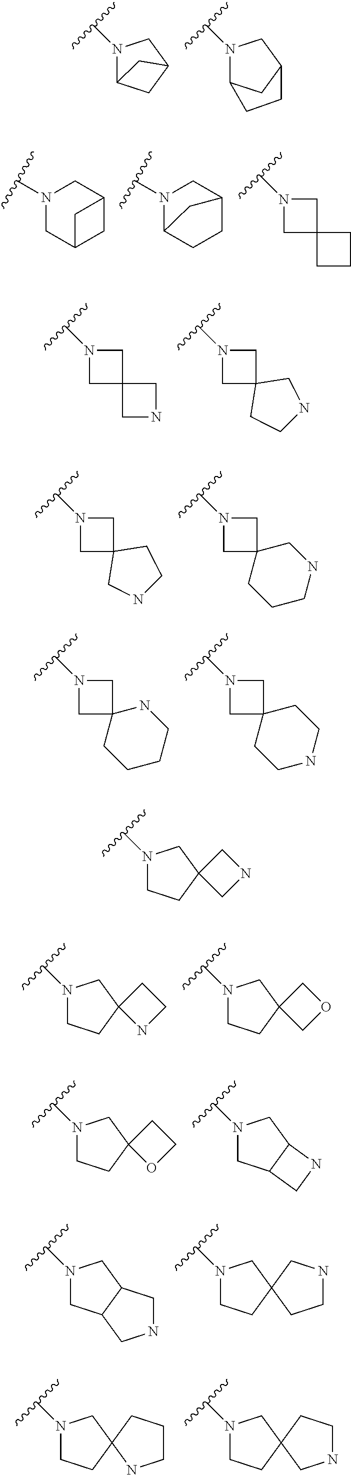

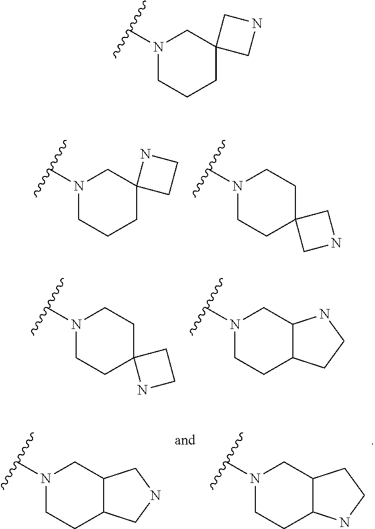

In some embodiments, L¹ is C₁-C₆ alkylene. For example, L¹ may be methylene, ethylene, or propylene. In some embodiments, L¹ is methylene.

In some embodiments, L¹ is C₁-C₄ alkylene substituted by one or two R⁴. For example, L¹ may be methylene substituted by one or two R⁴. In some embodiments, L¹ is C₁-C₄ alkylene substituted by one R⁴. In some embodiments, L¹ is C₁-C₄ alkylene substituted by two R⁴s. In some embodiments, R⁴ is C₁-C₃ alkyl (optionally substituted by one, two or three halogens). For example, R⁴ may each be independently selected from the group consisting of methyl, ethyl, and propyl. In other embodiments, two R⁴s together with the carbon to which the R⁴ are attached form a C₃-C₄ carbocyclyl. For example, two R⁴s together with the carbon to which the R⁴ are attached may form a cyclopropyl.

In other embodiments, L¹ is bond.

In some embodiments, R³ is H or C₁-C₆ alkyl. For example, R³ may be H or methyl. In some embodiments, R³ is H. In some embodiments, R³ is methyl.

In other embodiments, R³ is NR′R′, wherein R′ is each independently H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NR′R′, wherein R′ is each independently H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl. In some embodiments, R³ is NR′R′, wherein R′ is each independently H, C₁-C₆ alkyl (optionally substituted by one, two or three halogens). For example, R³ may be NH₂, NH(CH₃), or N(CH₃)₂.

In some embodiments, R³ is NH′R′ wherein R′ is H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NH′R′ wherein R′ is H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl.

In some embodiments, R³ is NH′R′ wherein R′ is C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NH′R′ wherein R′ is C₁-C₆ alkyl (optionally substituted by one, two or three halogens), C₁-C₆ alkylene-O—C₁-C₄ alkyl, or C₁-C₆ alkylene-SO₂—C₁-C₄ alkyl.

In other embodiments, R³ is 4-6 membered heterocyclyl optionally substituted by one or two substituents each independently selected from the group consisting of halo, hydroxyl, C₁-C₄ alkyl (optionally substituted by one, two or three halogens), and oxo. In some embodiments, R³ is 4-6 membered heterocyclyl comprising at least one nitrogen, wherein the heterocyclyl is optionally substituted by one or two substituents each independently selected from the group consisting of halo, hydroxyl, C₁-C₄ alkyl (optionally substituted by one, two or three halogens), and oxo. In some embodiments, R³ is a 4-membered heterocyclyl comprising at least one nitrogen, wherein the heterocyclyl is optionally substituted by one or two substituents each independently selected from the group consisting of halo, hydroxyl, C₁-C₄ alkyl (optionally substituted by one, two or three halogens), and oxo.

In some embodiments, R² is selected from the group consisting of H, F, CF₃, —C(O)—O-methyl, —C(O)OH, —O-methyl, C₃-C₇ cycloalkyl, and 4-6 membered heterocyclyl. In some embodiments, R² is selected from the group consisting of H, F, CF₃, —C(O)—O-methyl, —C(O)OH, —O-methyl, and C₃-C₇ cycloalkyl. In some embodiments, R² is selected from the group consisting of H, F, —O-methyl, and 4-membered heterocyclyl. In some embodiments, R² is selected from the group consisting of H, F, CF₃, —C(O)—O-methyl, —C(O)OH, and —O—methyl. In some embodiments, R² is H or F. In some embodiments, R² is H.

In some embodiments, R⁵ is H or methyl. In some embodiments, R⁵ is methyl. In some embodiments, R⁵ is H. In other embodiments, R² and R⁵ together with the carbon atoms to which R² and R⁵ are attached form a 5-membered carbocyclic ring.

A contemplated compound, for example, may be selected from the group consisting of:

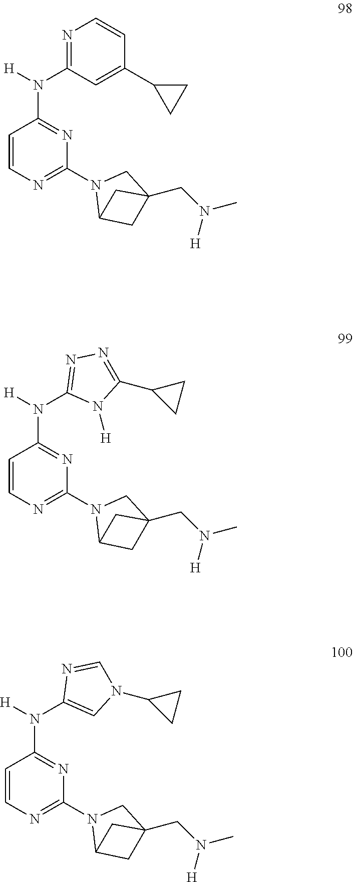 [SMILES: CNCC12CC(C1)N(c1nccc(Nc3cc(C4CC4)ccn3)n1)C2 ; CNCC12CC(C1)N(c1nccc(Nc3nnc(C4CC4)[nH]3)n1)C2 ; CNCC12CC(C1)N(c1nccc(Nc3cn(C4CC4)cn3)n1)C2]

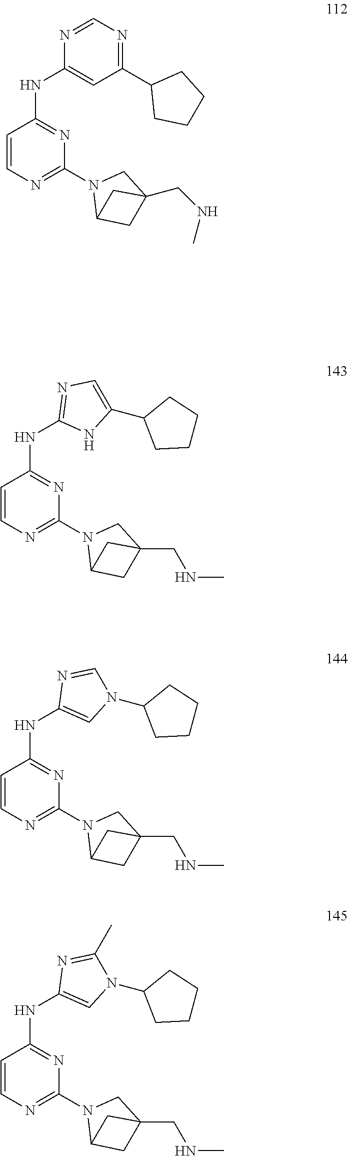 [SMILES: CNCC12CC(C1)N(c1nccc(Nc3cc(C4CCCC4)ncn3)n1)C2 ; CNCC12CC(C1)N(c1nccc(Nc3ncc(C4CCCC4)[nH]3)n1)C2 ; CNCC12CC(C1)N(c1nccc(Nc3cn(C4CCCC4)cn3)n1)C2 ; CNCC12CC(C1)N(c1nccc(Nc3cn(C4CCCC4)c(C)n3)n1)C2]

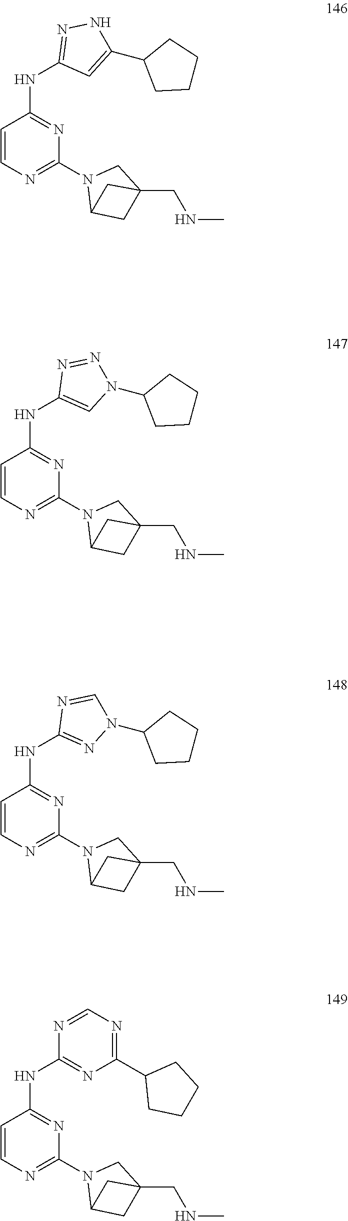 [SMILES: CNCC12CC(C1)N(c1nccc(Nc3cc(C4CCCC4)[nH]n3)n1)C2 ; CNCC12CC(C1)N(c1nccc(Nc3cn(C4CCCC4)nn3)n1)C2 ; CNCC12CC(C1)N(c1nccc(Nc3ncn(C4CCCC4)n3)n1)C2 ; CNCC12CC(C1)N(c1nccc(Nc3ncnc(C4CCCC4)n3)n1)C2]

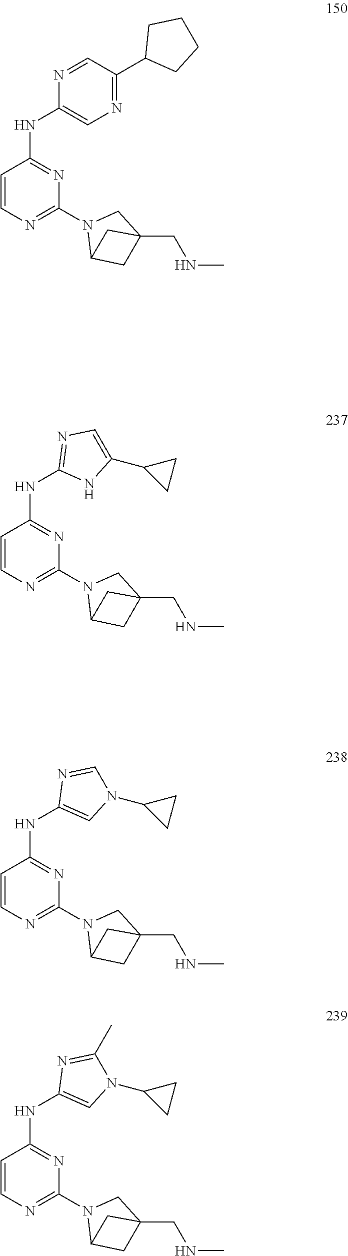 [SMILES: CNCC12CC(C1)N(c1nccc(Nc3cnc(C4CCCC4)cn3)n1)C2 ; CNCC12CC(C1)N(c1nccc(Nc3ncc(C4CC4)[nH]3)n1)C2 ; CNCC12CC(C1)N(c1nccc(Nc3cn(C4CC4)cn3)n1)C2 ; CNCC12CC(C1)N(c1nccc(Nc3cn(C4CC4)c(C)n3)n1)C2]

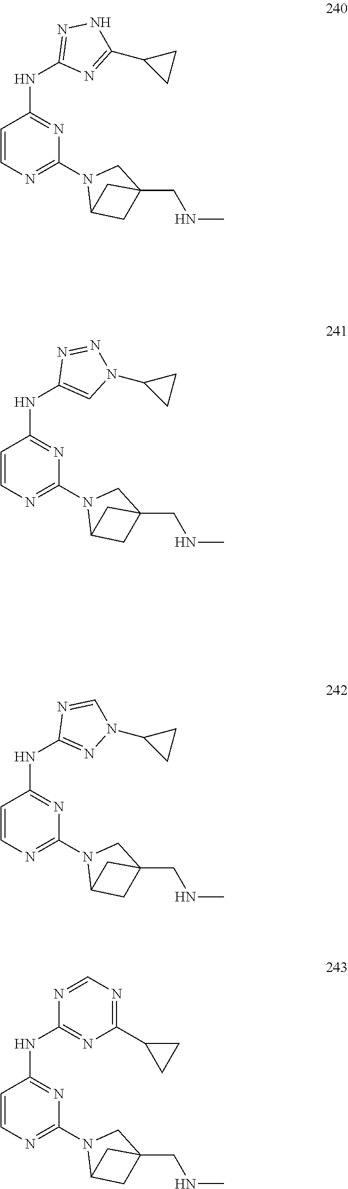 [SMILES: CNCC12CC(C1)N(c1nccc(Nc3n[nH]c(C4CC4)n3)n1)C2 ; CNCC12CC(C1)N(c1nccc(Nc3cn(C4CC4)nn3)n1)C2 ; CNCC12CC(C1)N(c1nccc(Nc3ncn(C4CC4)n3)n1)C2 ; CNCC12CC(C1)N(c1nccc(Nc3ncnc(C4CC4)n3)n1)C2]

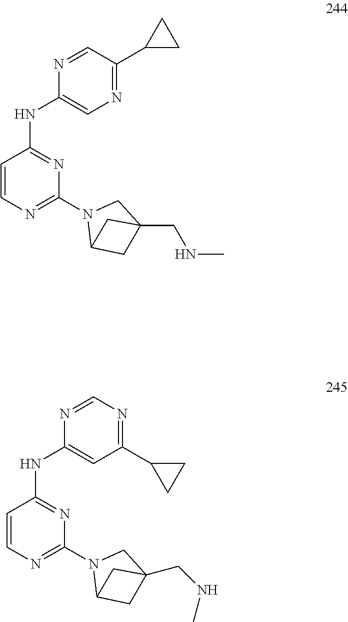 [SMILES: CNCC12CC(C1)N(c1nccc(Nc3cnc(C4CC4)cn3)n1)C2 ; CNCC12CC(C1)N(c1nccc(Nc3cc(C4CC4)ncn3)n1)C2]

or a pharmaceutically acceptable salt, stereoisomer, and/or N-oxide thereof.

Also provided herein, in some embodiments, are compounds represented by Formula III:

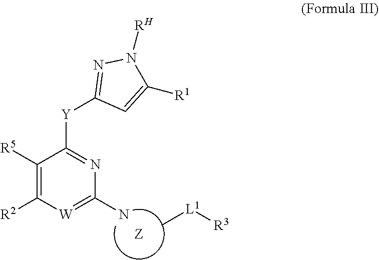

or a pharmaceutically acceptable salt, stereoisomer, and/or N-oxide thereof, wherein:
   W is selected from the group consisting of N, C—H, and C—F;
   Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
   ring Z is selected from the group consisting of 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, oxo, C₁-C₄alkoxy, and cyano;
   L¹ is bond or C₁-C₆ alkylene (optionally substituted by one, two or three R⁴);
   R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
   R⁴ is each independently selected from the group consisting of halogen and C₁-C₆ alkyl, or two R⁴ together with the carbon from which the R⁴ are attached form a C₃-C₅ carbocyclyl;
   R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
   R^{H} is selected from the group consisting of H, C₁-C₃alkyl, —C(O)—C₁-C₃alkyl, and C(O)—O—C₁-C₃alkyl;
   R^{N} is selected from the group consisting of H and C₁-C₃alkyl, or two R^{N} together with the nitrogen atoms to which the R^{N}s are attached form a 4-6 membered heterocyclic ring;
   R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
   R² is selected from the group consisting of H, F, CF₃, —C(O)—O-methyl, —C(O)OH, —O-methyl, —O-ethyl, C₃-C₇ cycloalkyl, and heterocyclyl, and
   R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring.

Exemplary contemplated compounds are represented by Formula III-A:

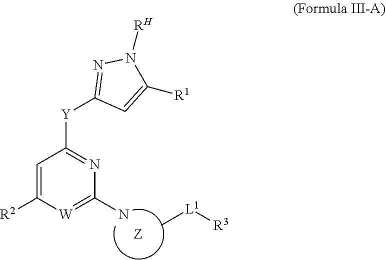

or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CF₂, CH(CH₃), C(CH₃)₂, and C(CH₂CH₂);
ring Z is selected from the group consisting of 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, and oxo;
L¹ is bond or C₁-C₆ alkylene (optionally substituted by one, two or three halogens);
R³ is selected from the group consisting of H, C₁-C₆ alkyl, NR′R′, and heterocyclyl optionally substituted by one or two substituents each independently selected from the group consisting of halo, hydroxyl, C₁-C₄ alkyl (optionally substituted by one, two or three halogens), and oxo;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, heterocyclyl, or heteroaryl may be substituted by one, two or three substituents each independently selected from halo and C₁-C₄alkyl (optionally substituted by one, two or three halogens); and
R² is selected from the group consisting of H, F, CF₃, —C(O)—O-methyl, —C(O)OH, —O-methyl, C₃-C₇ cycloalkyl, and heterocyclyl.

In some embodiments, W is C—H. In other embodiments, W is C—F. In other embodiments, W is N.

In some embodiments, R^{H} is selected from the group consisting of H, —C(O)—C₁-C₃alkyl, and C(O)—O—C₁-C₃alkyl. In some embodiments, R^{H} is H.

In some embodiments, for example, W is N, and the compound is represented by the Formula IIIa:

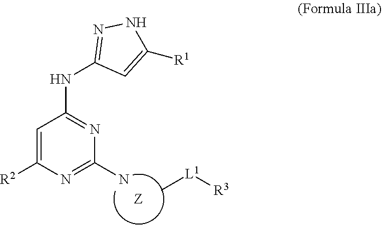

or a pharmaceutically acceptable salt, stereoisomer and/or N-oxide thereof.

In some embodiments, R¹ is a 5-6 membered heterocyclyl or C₃-C₆ cycloalkyl. In some embodiments, R¹ is a C₃-C₆ cycloalkyl. For example, R¹ may be cyclopropyl or cyclopentyl.

In some embodiments, ring Z is selected from the group consisting of 6-10 membered spiroheterocycle, a 6-10 membered fused bicyclic heterocyclic, and a 6-10 membered bridged cycloheteroalkyl, wherein Z may optionally be substituted by one or two substituents each independently selected from the group consisting of halo, hydroxyl, C₁-C₄ alkyl (optionally substituted by one, two or three halogens), and oxo.

In some embodiments, ring Z is selected from the group consisting of:

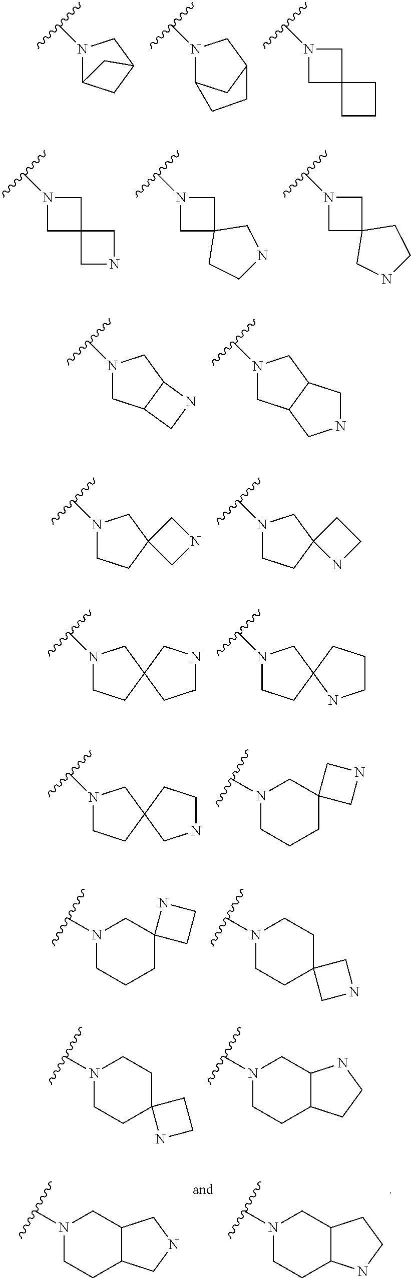

In some embodiments, ring Z is selected from the group consisting of:

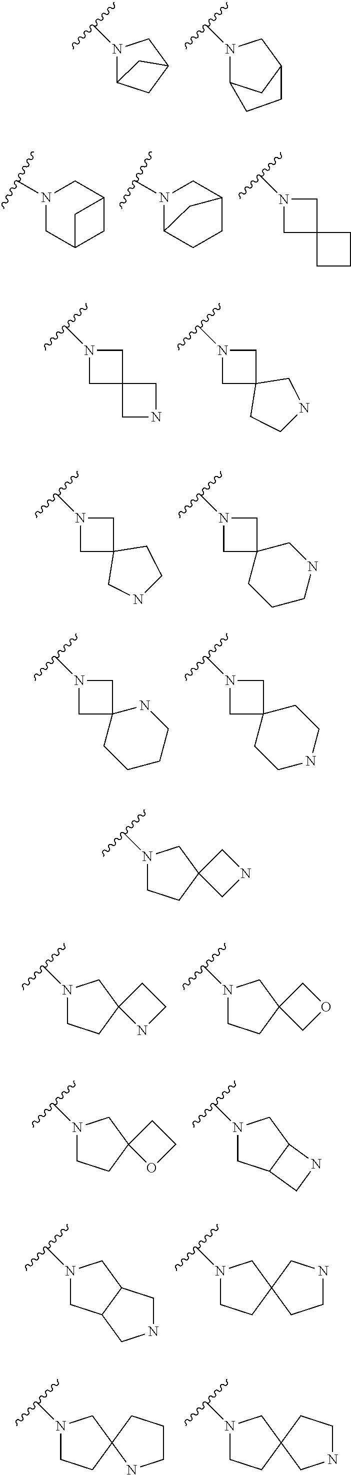

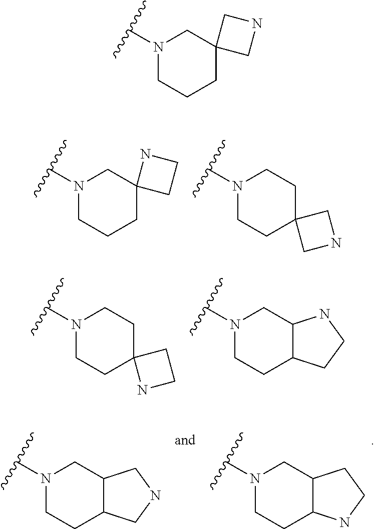

Exemplary contemplated compounds are represented by Formula IIIb:

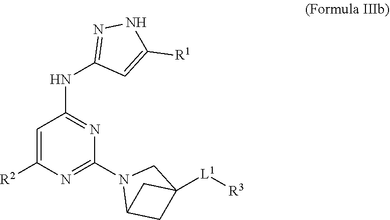

or a pharmaceutically acceptable salt, stereoisomer, and/or N-oxide thereof.

In some embodiments, L¹ is C₁-C₆ alkylene. For example, L¹ may be methylene, ethylene, or propylene. In some embodiments, L¹ is methylene.

In some embodiments, L¹ is C₁-C₄ alkylene substituted by one or two R⁴. For example, L¹ may be methylene substituted by one or two R⁴. In some embodiments, L¹ is C₁-C₄ alkylene substituted by one R⁴. In some embodiments, L¹ is C₁-C₄ alkylene substituted by two R⁴s. In some embodiments, R⁴ is C₁-C₃ alkyl (optionally substituted by one, two or three halogens). For example, R⁴ may each be independently selected from the group consisting of methyl, ethyl, and propyl. In other embodiments, two R⁴s together with the carbon to which the R⁴ are attached form a C₃-C₄ carbocyclyl. For example, two R⁴s together with the carbon to which the R⁴ are attached may form a cyclopropyl.

In other embodiments, L¹ is bond.

In some embodiments, R³ is H or C₁-C₆ alkyl. For example, R³ may be H or methyl. In some embodiments, R³ is H. In some embodiments, R³ is methyl.

In some embodiments, R³ is NR′R′, wherein R′ is each independently H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NR′R′, wherein R′ is each independently H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl. In some embodiments, R³ is NR′R′, wherein R′ is each independently H, C₁-C₆ alkyl (optionally substituted by one, two or three halogens). For example, R³ may be NH₂, NH(CH₃), or N(CH₃)₂.

In some embodiments, R³ is NH′R′ wherein R′ is H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NH′R′ wherein R′ is H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl.

In some embodiments, R³ is NH′R′ wherein R′ is C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NH′R′ wherein R′ is H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl.

In some embodiments, R³ is 4-6 membered heterocyclyl optionally substituted by one or two substituents each independently selected from the group consisting of halo, hydroxyl, C₁-C₄ alkyl (optionally substituted by one, two or three halogens), and oxo. In some embodiments, R³ is 4-6 membered heterocyclyl comprising at least one nitrogen, wherein the heterocyclyl is optionally substituted by one or two substituents each independently selected from the group consisting of halo, hydroxyl, C₁-C₄ alkyl (optionally substituted by one, two or three halogens), and oxo. In some embodiments, R³ is a 4-membered heterocyclyl comprising at least one nitrogen, wherein the heterocyclyl is optionally substituted by one or two substituents each independently selected from the group consisting of halo, hydroxyl, C₁-C₄ alkyl (optionally substituted by one, two or three halogens), and oxo.

In some embodiments, R² is selected from the group consisting of H, F, CF₃, —C(O)—O-methyl, —C(O)OH, —O-methyl, C₃-C₇ cycloalkyl, and 4-6 membered heterocyclyl. In some embodiments, R² is selected from the group consisting of H, F, CF₃, —C(O)—O-methyl, —C(O)OH, —O-methyl, and C₃-C₇ cycloalkyl. In some embodiments, R² is selected from the group consisting of H, F, —O-methyl, and 4-membered heterocyclyl. In some embodiments, R² is selected from the group consisting of H, F, CF₃, —C(O)—O-methyl, —C(O)OH, and —O—methyl. In some embodiments, R² is H or F. In some embodiments, R² is H.

In some embodiments, R⁵ is H or methyl. In some embodiments, R⁵ is methyl. In some embodiments, R⁵ is H. In other embodiments, R² and R⁵ together with the carbon atoms to which R² and R⁵ are attached form a 5-membered carbocyclic ring.

Other contemplated compounds of the present disclosure include those represented by Formula IV:

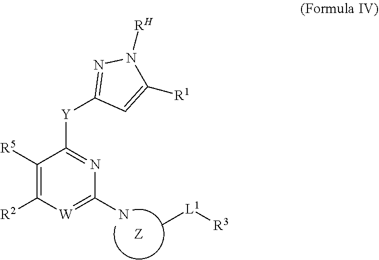

or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
ring Z is a 4-10 membered N-bound heterocyclic, wherein Z may optionally be substituted by one or two substituents each independently selected from the group consisting of halo, hydroxyl, C₁-C₄ alkyl (optionally substituted by one, two or three halogens), oxo, C₁-C₄alkoxy, and cyano;
L¹ is bond or C₁-C₆ alkylene (optionally substituted by one, two or three R⁴);
R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
R⁴ is each independently selected from the group consisting of halogen and C₁-C₆ alkyl, or two R⁴ together with the carbon from which the R⁴ are attached form a C₃-C₅ carbocyclyl;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R^{N} is selected from the group consisting of H and C₁-C₃alkyl, or two R^{N} together with the nitrogen atoms to which the R^{N}s are attached form a 4-6 membered heterocyclic ring;
R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
R² is selected from the group consisting of H, F, CF₃, —C(O)—O-methyl, —C(O)OH, —O-methyl, —O-ethyl, methyl, C₃-C₇ cycloalkyl, and heterocyclyl; and
R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring.

Exemplary contemplated compounds are represented by Formula IV-A:

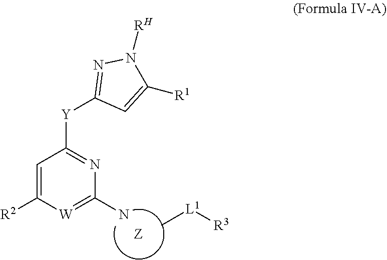

or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CF₂, CH(CH₃), C(CH₃)₂, and C(CH₂CH₂);
ring Z is a 4-10 membered N-bound heterocyclic, wherein Z may optionally be substituted by one or two substituents each independently selected from the group consisting of halo, hydroxyl, C₁-C₄ alkyl (optionally substituted by one, two or three halogens), and oxo;
L¹ is bond or C₁-C₆ alkylene (optionally substituted by one, two or three halogens);
R³ is selected from the group consisting of H, C₁-C₆ alkyl, NR′R′, and heterocyclyl optionally substituted by one or two substituents each independently selected from the group consisting of halo, hydroxyl, C₁-C₄ alkyl (optionally substituted by one, two or three halogens), and oxo;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₁₀ cycloalkyl, heterocyclyl, or heteroaryl may be substituted by one, two or three substituents each independently selected from halo and C₁-C₄alkyl (optionally substituted by one, two or three halogens); and
R² is selected from the group consisting of H, F, CF₃, —C(O)—O-methyl, —C(O)OH, —O-methyl, methyl, C₃-C₇ cycloalkyl, and heterocyclyl.

In some embodiments, W is C—H. In other embodiments, W is C—F. In other embodiments, W is N.

In some embodiments, R^{H} is selected from the group consisting of H, —C(O)—C₁-C₃alkyl, and C(O)—O—C₁-C₃alkyl. In some embodiments, R^{H} is H.

In some embodiments, for example, W is N, and having the Formula IVa:

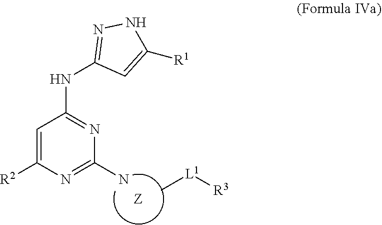

or a pharmaceutically acceptable salt, stereoisomer and/or N-oxide thereof.

In some embodiments, R¹ is a 5-6 membered heterocyclyl or C₃-C₆ cycloalkyl. In some embodiments, R¹ is a C₃-C₆ cycloalkyl. For example, R¹ may be cyclopropyl or cyclopentyl.

In some embodiments, ring Z is selected from the group consisting of 4-6 membered monocyclic heterocycle, 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, and oxo.

In some embodiments, ring Z is selected from the group consisting of 4-6 membered monocyclic heterocycle, 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), and oxo

In some embodiments, ring Z is selected from the group consisting of 6-10 membered spiroheterocycle, a 6-10 membered fused bicyclic heterocyclic, and a 6-10 membered bridged cycloheteroalkyl, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, and oxo.

In some embodiments, ring Z is selected from the group consisting of 6-10 membered spiroheterocycle, a 6-10 membered fused bicyclic heterocyclic, and a 6-10 membered bridged cycloheteroalkyl, wherein Z may optionally be substituted by one or two substituents each independently selected from the group consisting of halo, hydroxyl, C₁-C₄ alkyl (optionally substituted by one, two or three halogens), and oxo.

In some embodiments, ring Z is selected from the group consisting of:

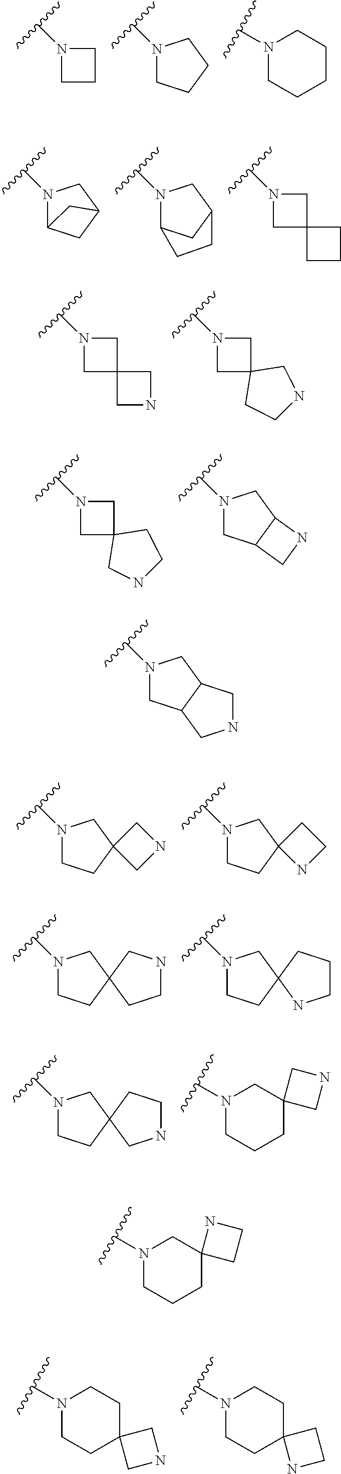

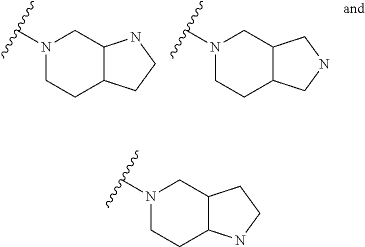

In some embodiments, ring Z is selected from the group consisting of:

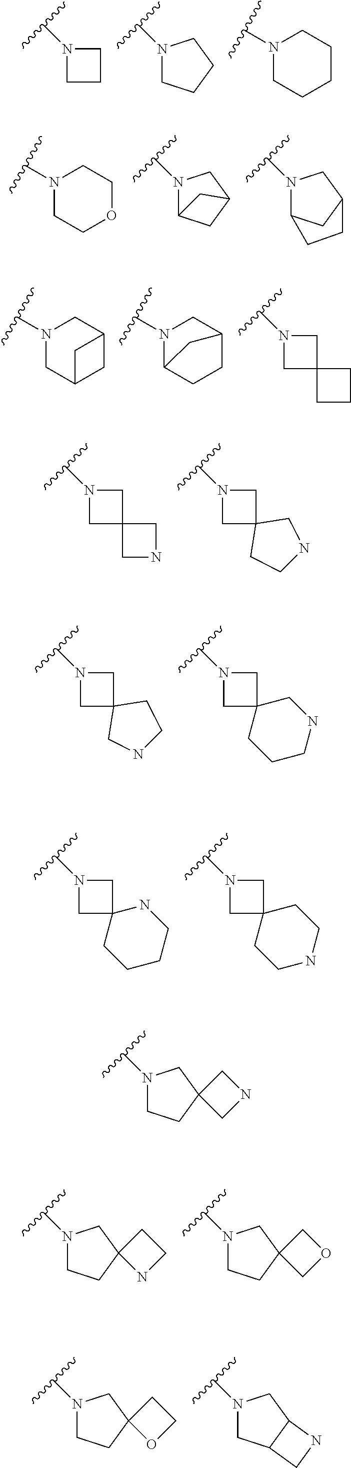

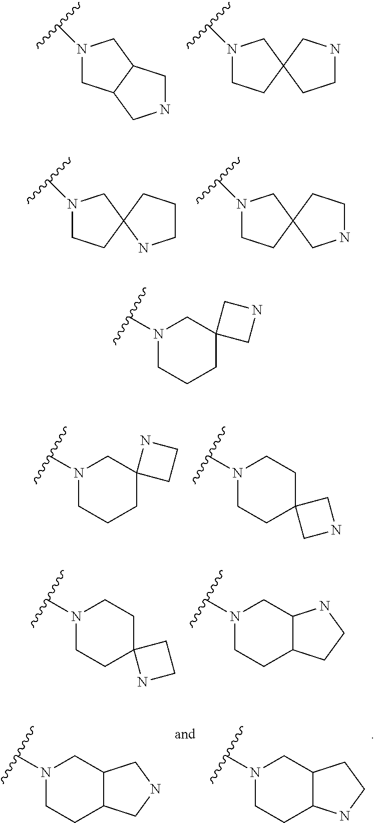

In some embodiments, ring Z is selected from the group consisting of:

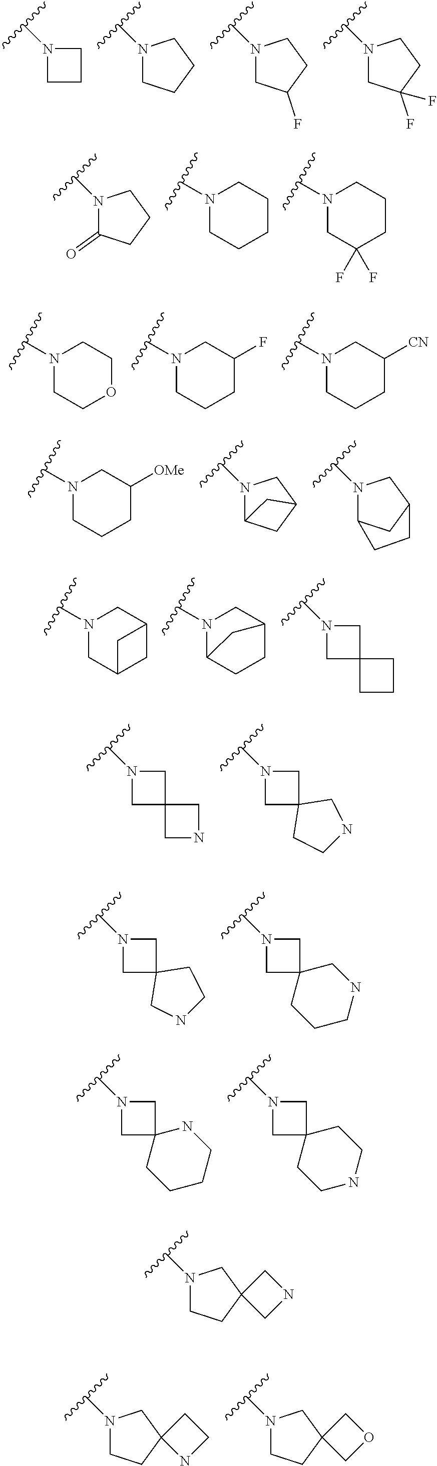

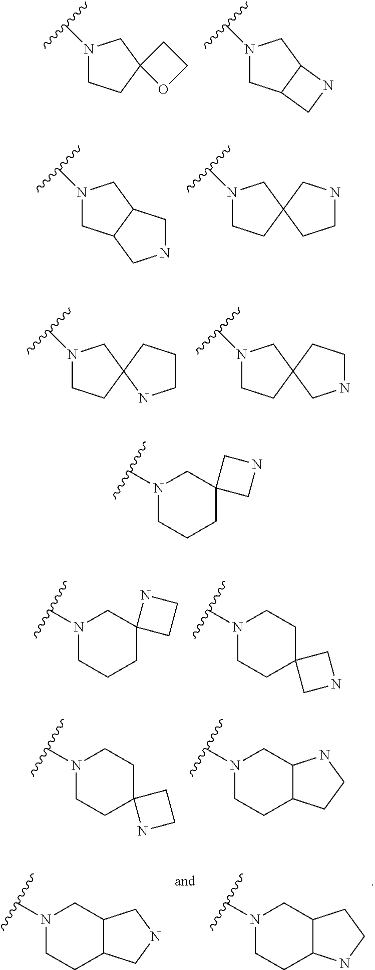

Exemplary contemplated compounds disclosed herein are represented by Formula IVb:

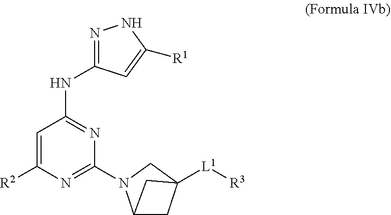

or a pharmaceutically acceptable salt, stereoisomer, and/or N-oxide thereof.

Also contemplated herein are compounds represented by Formula IVc:

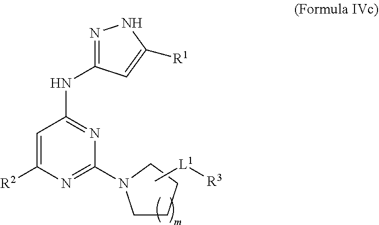

or a pharmaceutically acceptable salt, stereoisomer, and/or N-oxide thereof, wherein m is 0, 1, or 2.

Other exemplary contemplated compounds are represented by Formula IVd:

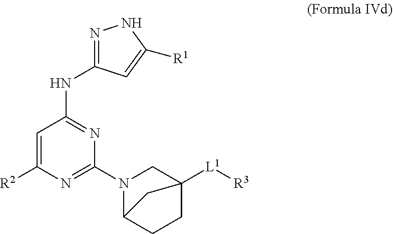

or a pharmaceutically acceptable salt, stereoisomer, and/or N-oxide thereof.

In some other embodiments, contemplated compounds are represented by Formula IVe:

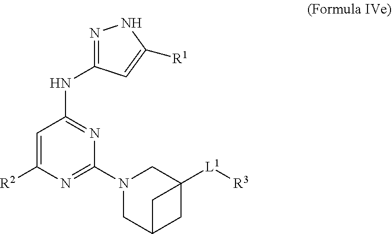

or a pharmaceutically acceptable salt, stereoisomer, and/or N-oxide thereof.

In some embodiments, L¹ is C₁-C₆ alkylene. For example, L¹ may be methylene, ethylene, or propylene. In some embodiments, L¹ is methylene.

In some embodiments, L¹ is C₁-C₄ alkylene substituted by one or two R⁴. For example, L¹ may be methylene substituted by one or two R⁴. In some embodiments, L¹ is C₁-C₄ alkylene substituted by one R⁴. In some embodiments, L¹ is C₁-C₄ alkylene substituted by two R⁴s. In some embodiments, R⁴ is C₁-C₃ alkyl (optionally substituted by one, two or three halogens). For example, R⁴ may each be independently selected from the group consisting of methyl, ethyl, and propyl. In other embodiments, two R⁴s together with the carbon to which the R⁴ are attached form a C₃-C₄ carbocyclyl. For example, two R⁴s together with the carbon to which the R⁴ are attached may form a cyclopropyl.

In other embodiments, L¹ is bond.

In some embodiments, R³ is H or C₁-C₆ alkyl. For example, R³ may be H or methyl. In some embodiments, R³ is H. In some embodiments, R³ is methyl.

In some embodiments, R³ is NR′R′, wherein R′ is each independently H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NR′R′, wherein R′ is each independently H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl. In some embodiments, R³ is NR′R′, wherein R′ is each independently H, C₁-C₆ alkyl (optionally substituted by one, two or three halogens). For example, R³ may be NH₂, NH(CH₃), or N(CH₃)₂.

In some embodiments, R³ is NH′R′ wherein R′ is H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NH′R′ wherein R′ is H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl.

In some embodiments, R³ is NH′R′ wherein R′ is C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R³ is NH′R′ wherein R′ is H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl.

In some embodiments, R³ is 4-6 membered heterocyclyl optionally substituted by one or two substituents each independently selected from the group consisting of halo, hydroxyl, C₁-C₄ alkyl (optionally substituted by one, two or three halogens), and oxo. In some embodiments, R³ is 4-6 membered heterocyclyl comprising at least one nitrogen, wherein the heterocyclyl is optionally substituted by one or two substituents each independently selected from the group consisting of halo, hydroxyl, C₁-C₄ alkyl (optionally substituted by one, two or three halogens), and oxo. In some embodiments, R³ is a 4-membered heterocyclyl comprising at least one nitrogen, wherein the heterocyclyl is optionally substituted by one or two substituents each independently selected from the group consisting of halo, hydroxyl, C₁-C₄ alkyl (optionally substituted by one, two or three halogens), and oxo.

In some embodiments, R² is selected from the group consisting of H, F, CF₃, —C(O)—O-methyl, —C(O)OH, —O-methyl, C₃-C₇ cycloalkyl, and 4-6 membered heterocyclyl. In some embodiments, R² is selected from the group consisting of H, F, CF₃, —C(O)—O-methyl, —C(O)OH, —O-methyl, and C₃-C₇ cycloalkyl. In some embodiments, R² is selected from the group consisting of H, F, —O-methyl, and 4-membered heterocyclyl. In some embodiments, R² is selected from the group consisting of H, F, CF₃, —C(O)—O-methyl, —C(O)OH, and —O—methyl. In some embodiments, R² is H or F. In some embodiments, R² is H.

In some embodiments, R⁵ is H or methyl. In some embodiments, R⁵ is methyl. In some embodiments, R⁵ is H. In other embodiments, R² and R⁵ together with the carbon atoms to which R² and R⁵ are attached form a 5-membered carbocyclic ring.

A contemplated compound, for example, may be selected from the group consisting of:

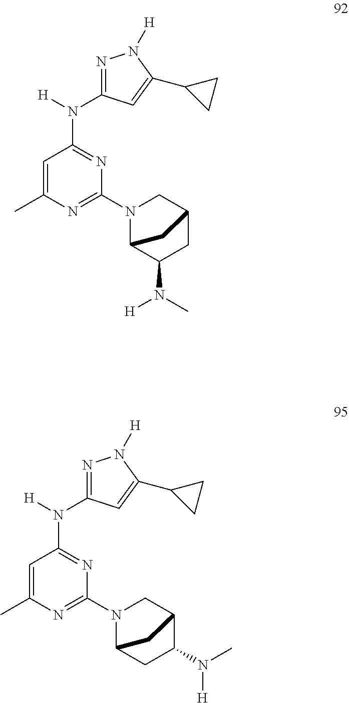

or a pharmaceutically acceptable salt, stereoisomer, and/or N-oxide thereof,
Also contemplated herein are compound represented by Formula V:

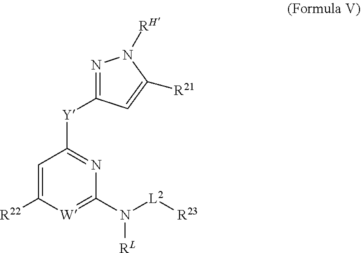

or a pharmaceutically acceptable salt, stereoisomer, and/or N-oxide thereof, wherein:
W′ is selected from the group consisting of N, C—H, and C—F;
Y′ is selected from the group consisting of NH, N—CH₃, O, S, CH₂, CFH, CF₂, CH(CH₃), C(CH₃)₂, C(CH₂CH₂). CHCF₃, CHCF₂H, CH(OCF₂H), CHOH, C(CH₃)OH, and CH(OCH₃);
R^{L} is H or C₁-C₆ alkyl;
L² is bond or C₁-C₆ alkylene (optionally substituted by one, two or three R²⁴);
R²³ is NR′R′;
R²⁴ is each independently selected from the group consisting of halogen and C₁-C₆ alkyl, or two R⁴ together with the carbon from which the R⁴ are attached form a C₃-C₅ carbocyclyl;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo; or two R's together with the nitrogen to which they are attached form a heterocyclyl which may optionally be substituted by methyl, halo, cyano, oxo, or hydroxyl;
R^{H′} is selected from the group consisting of H, C₁-C₃alkyl, —C(O)—C₁-C₃alkyl, and C(O)—O—C₁-C₃alkyl;
R²¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens); and
R²² is selected from the group consisting of H, F, —C(O)—O-methyl, —C(O)OH, —O-methyl, —O-ethyl, methyl, C₃-C₇ cycloalkyl, and heterocyclyl.

Exemplary contemplated compounds are represented by Formula V-A:

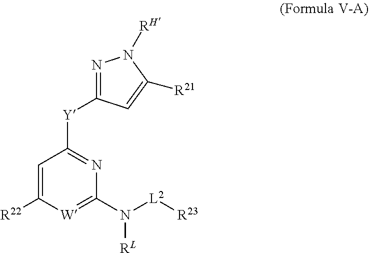

or a pharmaceutically acceptable salt, stereoisomer, and/or N-oxide thereof, wherein:
W′ is selected from the group consisting of N, C—H, and C—F;
Y′ is selected from the group consisting of NH, N—CH₃, O, S, CH₂, CF₂, CH(CH₃), C(CH₃)₂, and C(CH₂CH₂);
R^{L} is H or C₁-C₆ alkyl;
L² is bond or C₁-C₆ alkylene (optionally substituted by one, two or three halogens);
R²³ is NR′R′;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo; or two R's together with the nitrogen to which they are attached form a heterocyclyl which may optionally be substituted by methyl, halo, cyano, oxo, or hydroxyl;
R^{H′} is selected from the group consisting of H, C₁-C₃alkyl, —C(O)—C₁-C₃alkyl, and C(O)—O—C₁-C₃alkyl;
R²¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₁₀ cycloalkyl, heterocyclyl, or heteroaryl may be substituted by one, two or three substituents each independently selected from halo and C₁-C₄alkyl (optionally substituted by one, two or three halogens); and
R²² is selected from the group consisting of H, F, —C(O)—O-methyl, —C(O)OH, —O—methyl, methyl, C₃-C₇ cycloalkyl, and heterocyclyl.

In some embodiments, W′ is C—H. In other embodiments, W′ is C—F. In other embodiments, W′ is N.

In some embodiments, R^{H′} is selected from the group consisting of H, —C(O)—C₁-C₃alkyl, and C(O)—O—C₁-C₃alkyl. In some embodiments, R^{H′} is H.

In some embodiments, for example, W′ is N, and the compound is represented by the Formula Va:

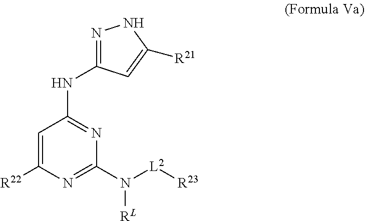

or a pharmaceutically acceptable salt, stereoisomer, and/or N-oxide thereof.

In some embodiments, R²¹ is a 5-6 membered heterocyclyl or C₃-C₆ cycloalkyl. In some embodiments, R²¹ is a C₃-C₆ cycloalkyl. For example, R²¹ may be cyclopropyl or cyclopentyl.

In some embodiments, L² is C₁-C₆ alkylene. For example, L² may be methylene, ethylene, or propylene. In some embodiments, L² is propylene. In other embodiments, L² is bond.

In some embodiments, R²³ is NR′R′, wherein R′ is each independently H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R²³ is NR′R′, wherein R′ is each independently H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl. In some embodiments, R²³ is NR′R′, wherein R′ is each independently H, C₁-C₆ alkyl (optionally substituted by one, two or three halogens). In some embodiments, R²³ is NH₂, NH(CH₃), or N(CH₃)₂.

In some embodiments, R²³ is NH′R′ wherein R′ is H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, or heterocyclyl optionally substituted by one or two substituents each independently selected from the group consisting of halo, hydroxyl, C₁-C₄ alkyl (optionally substituted by one, two or three halogens), and oxo. In some embodiments, R²³ is NH′R′ wherein R′ is H, C₁-C₆ alkyl (optionally substituted by one, two or three halogens), C₁-C₆ alkylene-O—C₁-C₄ alkyl, or C₁-C₆ alkylene-SO₂—C₁-C₄ alkyl.

In other embodiments, R²³ is NR′R′ and two R's together with the nitrogen to which they are attached form a 4-6 membered heterocyclyl.

In some embodiments, R²² is selected from the group consisting of H, F, CF₃, —C(O)—O-methyl, methyl, —C(O)OH, —O-methyl, and C₃-C₇ cycloalkyl. In some embodiments, R²² is selected from the group consisting of H, F, CF₃, —C(O)—O-methyl, —C(O)OH, —O—methyl, and methyl. In some embodiments, R²² is H, F, or methyl. In some embodiments, R²² is H or F. In some embodiments, R²² is H.

In some embodiments, R^{L} is H. In other embodiments, R^{L} is methyl.

A contemplated compound, for example, may be selected from the group consisting of:

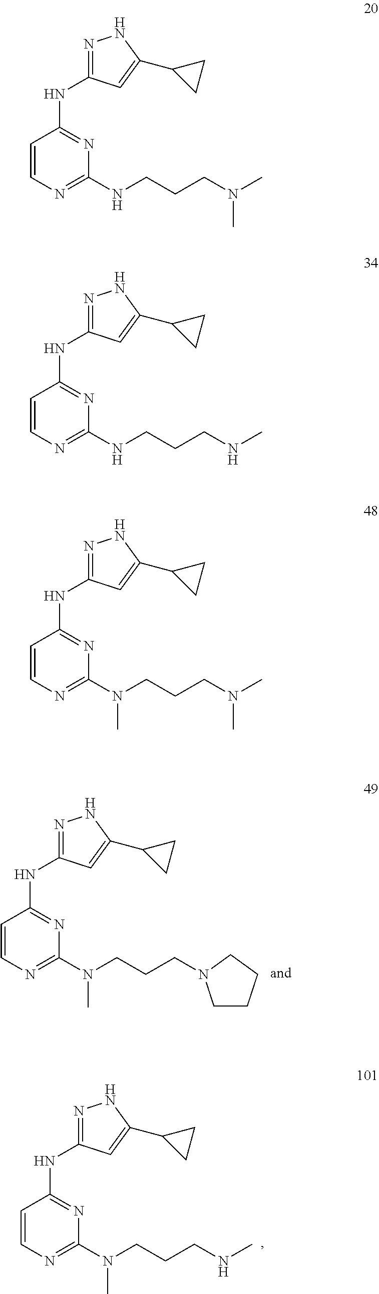 [SMILES: CN(C)CCCNc1nccc(Nc2cc(C3CC3)[nH]n2)n1 ; CNCCCNc1nccc(Nc2cc(C3CC3)[nH]n2)n1 ; CN(C)CCCN(C)c1nccc(Nc2cc(C3CC3)[nH]n2)n1 ; CN(CCCN1CCCC1)c1nccc(Nc2cc(C3CC3)[nH]n2)n1 ; CNCCCN(C)c1nccc(Nc2cc(C3CC3)[nH]n2)n1]

or a pharmaceutically acceptable salt, stereoisomer, and/or N-oxide thereof.

Disclosed compounds described herein may be present in a salt form, and the salt form of the compound is a pharmaceutically acceptable salt, and/or compounds described herein may be present in a prodrug form. Any convenient prodrug forms of the subject compounds can be prepared, for example, according to the strategies and methods described by Rautio et al. (“Prodrugs: design and clinical applications”, Nature Reviews Drug Discovery 7, 255-270 (February 2008)). Compounds described herein may be present in a solvate form.

In some embodiments, the compounds, or a prodrug form thereof, are provided in the form of pharmaceutically acceptable salts. Compounds containing an amine functional group or a nitrogen-containing heteroaryl group may be basic in nature and may react with any number of inorganic and organic acids to from the corresponding pharmaceutically acceptable salts. Inorganic acids commonly employed to form such salts include hydrochloric, hydrobromic, hydroiodic, sulfuric, and phosphoric acids, and related inorganic acids. Organic acids commonly employed to form such salts include para-toluenesulfonic, methanesulfonic, oxalic, para-bromophenylsulfonic, fumaric, maleic, carbonic, succinic, citric, benzoic and acetic acid, and related organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the related salts.

It is understood that all variations of salts, solvates, hydrates, prodrugs and stereoisomers are meant to be encompassed by the present disclosure.

### Pharmaceutical Compositions and Formulations

The compounds, prodrugs, and compositions described herein can be useful as pharmaceutical compositions for administration to a subject in need thereof.

Accordingly, pharmaceutical compositions are presented that can comprise at least a compound described herein, a pharmaceutically acceptable salt thereof, or a prodrug thereof, and at least one pharmaceutically acceptable carriers, diluent, stabilizers, excipients, dispersing agents, suspending agents, or thickening agents. For example, a disclosed pharmaceutical compositions may include one or more of the disclosed compounds, pharmaceutically acceptable salts, or prodrugs described herein. Contemplated compositions may include a compound, a pharmaceutically acceptable salt thereof, or a prodrug thereof in a therapeutically effective amount, for example, a disclosed pharmaceutical composition may be formulated for parenteral administration to a subject in need thereof, formulated for intravenous administration to a subject in need thereof, or formulated for subcutaneous administration to a subject in need thereof.

### Methods of Treatment

As described above, embodiments of the present disclosure include the use of compounds, prodrugs, and pharmaceutical compositions described herein to treat a Myc protein associated proliferative disease in a subject in need thereof. Such proliferative diseases include cancer, for example, a cancer selected from a group consisting of head and neck cancer, nervous system cancer, brain cancer, neuroblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genito-urinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site.

In some embodiments, a contemplated method of treating includes treating a cancer that is a Myc protein associated cancer, e.g., wherein the Myc protein is selected from the group consisting of a N-Myc protein, a c-MYc protein, a L-Myc protein, a human N-Myc protein, a human c-Myc protein, and a human L-Myc protein.

For example, provided herein is a method of treating a cancer selected from the group consisting of neuroblastoma, small cell lung carcinoma, breast cancer or a hematopoietic cancer.

In some embodiments, a disclosed method to treat cancer further comprises a second therapy, wherein the secondary therapy is an antineoplastic therapy, e.g., a contemplated method may further comprise administering an antineoplastic therapy such as one or more agents selected from a DNA topoisomerase I or II inhibitor, a DNA damaging agent, an immunotherapeutic agent (e.g., an antibody, cytokine, immune checkpoint inhibitor or cancer vaccine), an antimetabolite or a thymidylate synthase (TS) inhibitor, a microtubule targeted agent, ionizing radiation, an inhibitor of a mitosis regulator or a mitotic checkpoint regulator, an inhibitor of a DNA damage signal transducer, and an inhibitor of a DNA damage repair enzyme. For example, additional antineoplastic therapy may be selected from the group consisting of immunotherapy (e.g., immuno-oncologic therapy), radiation therapy, photodynamic therapy, gene-directed enzyme prodrug therapy (GDEPT), antibody-directed enzyme prodrug therapy (ADEPT), gene therapy, and controlled diets.

The present disclosure also contemplates the use of compounds, prodrugs, and pharmaceutical compositions described herein to modulate the amount and activity of a Myc protein (in vitro or in a patient), where the Myc protein may be for example a N-Myc protein, a c-MYc protein, a L-Myc protein, a human N-Myc protein, a human c-Myc protein, and/or a human L-Myc protein.

For example, the disclosure provides a method of modulating the amount (e.g., the concentration) and/or activity of a Myc protein such as (e.g. degrading a Myc protein, or modulating the rate of degradation of a Myc protein) that comprises contacting a Myc protein with an effective amount of a compound described herein, or a pharmaceutically acceptable salt, stereoisomer, and/or N-oxide thereof, including embodiments or from any examples, tables or figures.

Contemplated methods include methods of modulating the protein-protein interactions of the Myc family protein, or a method of decreasing the amount and decreasing the level of activity of a Myc protein.

A disclosed method of modulating the amount and activity of a Myc protein may include co-administering a compound described herein, or a pharmaceutically acceptable salt thereof, and a therapeutically effective amount of a second agent, e.g., therapeutic agent.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (i.e., amounts, temperatures, etc.), but some experimental error and deviation should, of course, be allowed for.

### General Experimental

¹H and ¹³C NMR spectra were recorded in CDCl₃ (residual internal standard CHCl₃=δ 7.26), DMSO-d₆ (residual internal standard CD₃SOCD₂H=δ 2.50), methanol-d₄ (residual internal standard CD₂HOD=δ 3.20), or acetone-d₆ (residual internal standard CD₃COCD₂H=δ 2.05). The chemical shifts (δ) reported are given in parts per million (ppm) and the coupling constants (J) are in Hertz (Hz). The spin multiplicities are reported as s=singlet, bs=broad singlet, bm=broad multiplet, d=doublet, t=triplet, q=quartet, p=pentuplet, dd=doublet of doublet, ddd=doublet of doublet of doublet, dt=doublet of triplet, td=triplet of doublet, tt=triplet of triplet, and m=multiplet.

UPLC-MS analysis was carried out on a Waters Acquity UPLC system consisting of an Acquity I—Class Sample Manager—FL, Acquity I—Class Binary Solvent Manager and an Acquity UPLC Column Manager. UV detection was afforded using an Acquity UPLC PDA detector (scanning from 210 to 400 nm), whilst mass detection was achieved using aa Acquity QDa detector (mass scanning from 100-1250 Da; positive and negative modes simultaneously). A Waters Acquity UPLC BEH C18 column (2.1×50 mm, 1.7 mm) was used to separate the analytes.

Samples were prepared by dissolution (with or without sonication) into 1 mL of 50% (v/v) MeCN in water. The resulting solutions were then filtered through a 0.2 mm syringe filter before submitting for analysis. All of the solvents, including formic acid and 36% ammonia solution, were purchased as the HPLC grade. Conditions (Acidic 2 min): 0.1% v/v Formic acid in water [Eluent A]; 0.1% v/v Formic acid in MeCN [Eluent B]; Flow rate 0.8 mL/min; injection volume 2 mL and 1.5 min equilibration time between samples. Conditions (Acidic 4 min): 0.1% v/v formic acid in water [Eluent A]; 0.1% v/v formic acid in MeCN [Eluent B]; Flow rate 0.8 mL/min; injection volume 2 mL and 1.5 min equilibration time between samples. Conditions (Basic 2 min): 0.1% ammonia in water [Eluent A]; 0.1% ammonia in MeCN [Eluent B]; Flow rate 0.8 mL/min; injection volume 2 mL and 1.5 min equilibration time between samples. Conditions (Basic 4 min): 0.1% ammonia in water [Eluent A]; 0.1% ammonia in MeCN [Eluent B]; Flow rate 0.8 mL/min; injection volume 2 mL and 1.5 min equilibration time between samples.

### General Synthetic Schemes

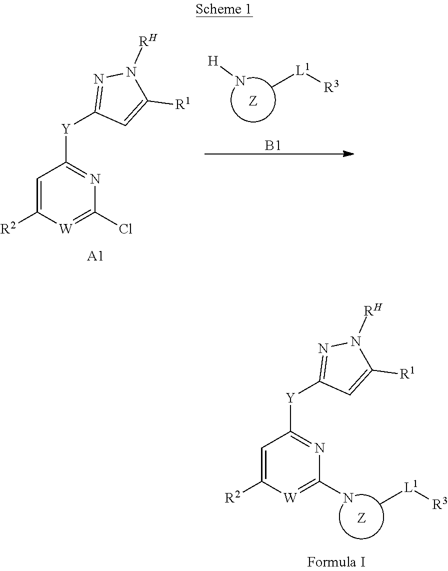

In certain embodiments, a compound of Formula I is prepared according to Scheme 1. Intermediate A1 (e.g., disclosed herein) is reacted with amine B1 (e.g., amines disclosed herein) in the presence of a base (e.g., N,N-diisopropylethylamine) at elevated temperatures to produce a compound of Formula I. In certain embodiments, amine B1 may comprise a protecting group (e.g., tert-butyloxycarbonyl (Boc), benzyl carbamate (Cbz)). In certain embodiments, when amine B1 comprises a protecting group (e.g., tert-butyloxycarbonyl (Boc), benzyl carbamate (Cbz)), the intermediate produced from the reaction thereof with intermediate A1 in the presence of a base may further be deprotected (e.g., in the presence of reducing agents such as hydrogen (e.g., with Pd/C as catalyst) or LiAlH₄ or acid such as TFA or HBr) to produce a compound of Formula I.

### Synthesis of Intermediates 2-Chloro-N-(5-cyclopropyl-1H-pyrazol-3-yl)pyrimidin-4-amine

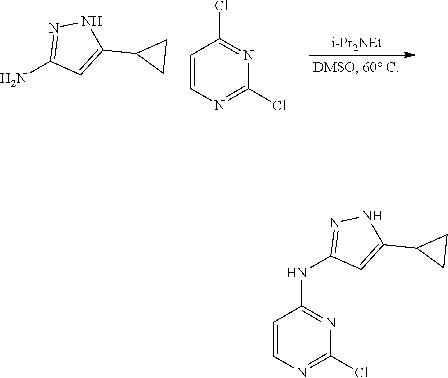

A solution of 5-cyclopropyl-1H-pyrazol-3-amine (4.55 g, 36.9 mmol), 2,4-dichloropyrimidine (5.00 g, 33.6 mmol) and N,N-diisopropylethylamine (8.8 mL, 50 mmol) in DMSO (50 mL) was heated to 60° C. and stirred for 16 hours. The reaction mixture was then cooled to ambient temperature, then added to ice water (ca. 1 L). The precipitate was collected by filtration, then dried under reduced pressure to afford the title compound as a solid (7.41 g, 31.4 mmol, 94%).

¹H NMR (400 MHz, DMSO-d₆) δ 12.17 (s, 1H), 10.26 (s, 1H), 8.14 (s, 1H), 7.31 (s, 1H), 5.93 (s, 1H), 1.89 (tt, J=8.7, 5.2 Hz, 1H), 1.01-0.88 (m, 2H), 0.68 (q, J=3.5, 1.9 Hz, 2H).

UPLC-MS (basic method, 2 min): Rt: 0.87 min, m/z: 236.2 [M+H]⁺.

### Synthesis of 2-Chloro-N-(5-cyclopentyl-1H-pyrazol-3-yl)pyrimidin-4-amine

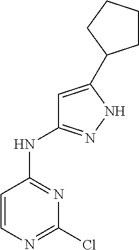 [SMILES: Clc1nccc(Nc2cc(C3CCCC3)[nH]n2)n1]

A solution of 2,4-dichloropyrimidine (450 mg, 3.0 mmol, 1.0 eq) in anhydrous DMSO (10.0 mL) was treated sequentially with 5-cyclopentyl-1H-pyrazol-3-amine (502 mg, 3.2 mmol, 1.10 eq) and N,N-diisopropylethylamine (0.79 mL, 4.5 mmol, 1.50 eq), and the resulting solution was stirred at 60° C. for 20 h. The reaction mixture was cooled to ambient temperature and poured into ice water, affording a suspension, which was stirred for 5 min. The mixture was filtered to afford a solid, which was washed with water (50 mL) before being dried under vacuum to constant weight to give the desired compound as a solid (675 mg, 2.56 mmol, 85%). UPLC-MS (Basic Method, 2 min): rt 0.98 min, m/z=263.3 [M+H]⁺. 1H NMR (400 MHz, DMSO) δ 12.19 (s, 1H), 10.31 (s, 1H), 8.16 (s, 1H), 3.02 (q, J=8.3 Hz, 1H), 2.05-1.94 (m, 2H), 1.74-1.66 (m, 2H), 1.58 (ddt, J=20.3, 15.7, 7.0 Hz, 4H).

### Synthesis of tert-Butyl 4-(Methylsulfonyloxymethyl)-2 azabicyclo[2.1.1]hexane-2-carboxylate

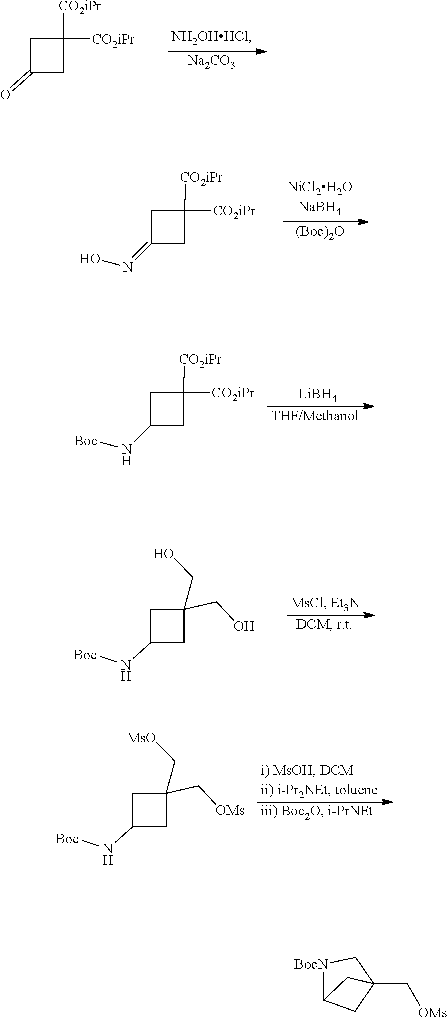

### Diisopropyl 3-hydroxyiminocyclobutane-1,1-dicarboxylate

Diisopropyl 3-oxocyclobutane-1,1-dicarboxylate (50.00 g, 206 mmol) and hydroxylamine hydrochloride (28.68 g, 413 mmol) were dissolved in ethanol (400 mL) and water (400 mL) mixture. Sodium carbonate (65.62 g, 619 mmol) was added, and the mixture heated to reflux for 1 hour. Reaction was allowed to cool to room temperature and volatiles were removed under reduced pressure. The obtained residue was suspended in water (150 mL), cooled to 5° C. and filtered. The resultant white solids were dried in the vacuum oven at 40° C. for 2 hours. The solid was then triturated in heptane (250 mL) and filtered to give the title compound as a solid (44.50 g, 173 mmol, 84%).

¹H NMR (400 MHz, Chloroform-d) δ 8.20-7.99 (m, 1H), 5.13-4.99 (m, 2H), 3.45-3.34 (m, 4H), 1.27-1.19 (m, 12H).

UPLC MS-ELS (2 minute, basic method, ELSD) rt=1.02 min, m/z=258.1 [M+H⁺].

### Diisopropyl 3-(tert-butoxycarbonylamino)cyclobutane-1,1-dicarboxylate

To a suspension of di-tert-butyl dicarbonate (22.56 g, 103 mmol) and nickel(II) chloride hexahydrate (6.14 g, 25.8 mmol) in 1-butanol (130 mL) was added a solution of diisopropyl 3-hydroxyiminocyclobutane-1,1-dicarboxylate (13.30 g, 51.7 mmol) in methanol (70 mL) at 0° C. followed by sodium borohydride (7.82 g, 207 mmol) in a portion wise manner. The reaction mixture was warmed to room temperature and stirred for 16 hours. Diethylene triamine (13 mL, 119 mmol) was added to it and the reaction was stirred for 1 hour. The reaction mixture was diluted with water (130 mL) and i-PrOAc (260 mL). After stirring for 15 minutes, the organic layer was separated, and aqueous layer was extracted with i-PrOAc (130 mL). The combined organic layers were, washed with brine (200 mL), dried over sodium sulfate, and concentrated under reduced pressure to afford the title compound as an oil, which solidified upon standing (16.20 g, 47.2 mmol, 91%).

¹H NMR (400 MHz, Chloroform-d) δ 5.10-4.96 (m, 2H), 4.83 (d, J=8.7 Hz, 1H), 2.88 (d, J=13.4 Hz, 2H), 2.40-2.28 (m, 2H), 1.43 (d, J=2.1 Hz, 1H), 1.41 (s, 9H), 1.29-1.14 (m, 12H).

UPLC MS (2 minute, basic method, ELSD) rt=1.24 min, m/z=342.2 [M−H]⁻.

### tert-Butyl N-[3,3-Bis(hydroxymethyl)cyclobutyl]carbamate

To diisopropyl 3-(tert-butoxycarbonylamino)cyclobutane-1,1-dicarboxylate (17.9 g, 52.1 mmol) in THE (270 mL) was added lithium borohydride (4.54 g, 208 mmol). Reaction mixture was warmed to 50° C. and methanol (54 mL) added to the reaction mixture in a dropwise manner over 10 min. Once effervescence had subsided, the reaction was heated at reflux for 1 hour. The reaction mixture was then cooled to room temperature and 0.5 M aqueous citric acid (100 mL) was added. After stirring for 16 hours, the reaction mixture was transferred to separatory funnel with water (300 mL) and EtOAc (400 mL). The aqueous layer was further extracted with EtOAc (2×100 mL). The combined organic extracts were dried over Na₂SO₄ and concentrated to dryness under reduced pressure. The crude material was purified by triturating in ether to afford the title compound as a solid (11.0 g, 47.6 mmol, 91%).

¹H NMR (400 MHz, Methanol-d₄) δ 4.14-3.78 (m, 1H), 3.55 (s, 2H), 3.46 (s, 2H), 2.17-2.06 (m, 2H), 1.79-1.67 (m, 2H), 1.40 (s, 9H).

### [3-(tert-Butoxycarbonylamino)-1-(methylsulfonyloxymethyl)cyclobutyl]methyl methanesulfonate

In a round bottom flask, tert-butyl N-[3,3-bis(hydroxymethyl)cyclobutyl]carbamate (10.70 g, 46.3 mmol) and triethylamine (26 mL, 185 mmol) was suspended in chloroform (100 mL). The reaction mixture was cooled to 0° C. Afterwards, methanesulfonyl chloride (7.9 mL, 102 mmol) was added dropwise, and reaction was allowed to warm to ambient temperature and stirred for 16 hours. The reaction mixture was transferred the reaction mixture to separating funnel with water (200 mL) and DCM (300 mL The layers were separated, then the aqueous layer was further extracted with DCM (2×200 mL). The combined organic extracts were washed with brine (200 mL) and dried over Na₂SO₄ then concentrated to dryness under reduced pressure. The residue was purified by normal-phase flash column chromatography eluting with a gradient of 0 to 60% EtOAc in iso-hexane to afford the title compound as a solid (7.00 g, 18.1 mmol, 39%).

¹H NMR (400 MHz, Chloroform-d) δ 4.77 (s, 1H), 4.25 (d, J=0.7 Hz, 2H), 4.19 (d, J=0.7 Hz, 2H), 3.08-3.00 (m, 6H), 2.41-2.31 (m, 2H), 2.05-1.91 (m, 2H), 1.41 (s, 9H).

### tert-Butyl 4-(Methylsulfonyloxymethyl)-2-azabicyclo[2.1.1]hexane-2-carboxylate

To a solution of [3-(tert-butoxycarbonylamino)-1-(methylsulfonyloxymethyl)cyclobutyl]methyl methanesulfonate (7.00 g, 18.1 mmol) in DCM (100 mL) was added methanesulfonic acid (3.6 mL, 54 mmol) and reaction mixture was stirred for 2 hours at room temperature. THE (30 mL) was added and stirring was continued for another 2 hours. Afterwards, the volatiles were removed under reduced pressure to afford semi-solid residue. The residue was taken in toluene (50 mL) and N,N-diisopropylethylamine (14 mL, 81 mmol), then IPA (25 mL) was added and reaction was heated to reflux and stirred for 16 hours. The reaction mixture was then cooled to room temperature, then N,N-diisopropylethylamine (6.3 mL, 36.1 mmol) and di-tert-butyl dicarbonate (5.91 g, 27.1 mmol) added successively. After stirring of 16 hours at room temperature, the reaction mixture was transferred to separatory funnel with water (400 mL) and EtOAc (200 mL). The organic layer was separated and the aqueous layer was further extracted with EtOAc (300 mL). The combined organics were washed with brine (400 mL), dried over Na₂SO₄ and concentrated under reduced pressure to afford an oily residue. The crude material was purified by normal-phase column chromatography, over silica gel, eluting with a gradient of EtOAc (50% to 100%; v/v) in iso-hexane to afford the title compound as a solid (2.50 g, 8.58 mmol, 48%).

¹H NMR (400 MHz, Chloroform-d) δ 4.42-4.34 (m, 3H), 3.28 (s, 2H), 3.02 (s, 3H), 1.92-1.82 (m, 2H), 1.63-1.47 (m, 2H), 1.46 (s, 9H).

### Example 1. Synthesis of Compound 1

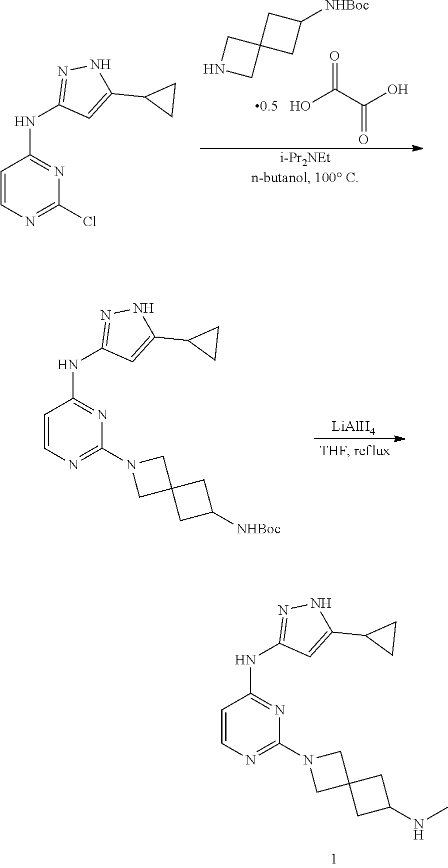

### tert-Butyl (2-(4-((5-Cyclopropyl-1H-pyrazol-3-yl)amino)pyrimidin-2-yl)-2-azaspiro[3.3]heptan-6-yl)carbamate

A solution of 2-chloro-N-(5-cyclopropyl-1H-pyrazol-3-yl)pyrimidin-4-amine (400 mg, 1.69 mmol), tert-butyl N-(2-azaspiro[3.3]heptan-6-yl)carbamate hemioxalate (873 mg, 3.39 mmol) and N,N-diisopropylethylamine (0.888 mL, 5.10 mmol) in n-butanol (15 mL) was heated to 100° C. and stirred for 44 hours. The reaction mixture was then cooled to ambient temperature, and the volatiles were removed under reduced pressure. The crude residue was triturated with a 1:1 mixture of EtOAc and water to afford the title compound as a solid (430 mg, 0.961 mmol, 57%).

¹H NMR (400 MHz, DMSO-d₆) δ 9.80 (s, 1H), 7.82 (d, J=6.0 Hz, 1H), 7.12 (d, J=7.9 Hz, 1H), 6.33 (s, 1H), 6.18 (s, 1H), 4.04 (s, 2H), 3.93 (s, 2H), 3.85 (d, J=7.7 Hz, 1H), 2.44 (t, J=10.0 Hz, 3H), 2.10 (dd, J=11.8, 8.6 Hz, 2H), 1.95-1.77 (m, 1H), 1.37 (s, 11H), 1.26 (t, J=6.2 Hz, 4H), 0.97-0.88 (m, 2H), 0.67 (d, J=5.3 Hz, 2H).

UPLC-MS (basic method, 4 min): Rt: 1.01 min, m/z: 412.3 [M+H]⁺.

### 2-(4-((5-cyclopropyl-1H-pyrazol-3-yl)amino)pyrimidin-2-yl)-N-methyl-2-azaspiro[3.3]heptan-6-amine

To a solution of tert-butyl (2-(4-((5-cyclopropyl-1H-pyrazol-3-yl)amino)pyrimidin-2-yl)-2-azaspiro[3.3]heptan-6-yl)carbamate (150 mg, 0.365 mmol) in anhydrous THE (25 mL) was added a 2.4 M solution of lithium aluminum hydride in THE (1.5 mL, 3.6 mmol) dropwise at 0° C. The reaction mixture was heated to reflux and stirred for 2 hours. The reaction mixture was then cooled to 0° C., then quenched with water (140 μL), then a 15% w/v NaOH solution (140 μL), followed by water (420 μL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (10 mL). The eluent was then concentrated to dryness under reduced pressure. The crude residue was purified by reversed-phase acidic prepHPLC, using TFA as the additive, followed by reversed-phase basic prepHPLC, using ammonia as the additive, to afford the title compound as a solid (27 mg, 79 μmol, 22%).

¹H NMR (400 MHz, DMSO-d₆) δ 11.90 (s, 1H), 9.44 (s, 1H), 7.82 (d, J=5.7 Hz, 1H), 6.29 (s, 1H), 6.13 (s, 1H), 3.96 (s, 2H), 3.86 (s, 2H), 3.10-2.83 (m, 1H), 2.36 (t, J=9.5 Hz, 2H), 2.15 (s, 4H), 1.86 (m, 3H), 1.04 (d, J=6.1 Hz, 1H), 0.90 (d, J=8.2 Hz, 2H), 0.65 (d, J=5.0 Hz, 2H).

UPLC-MS (basic method, 4 min): Rt: 1.10 min, m/z: 326.3 [M+H]⁺.

### Example 2. Synthesis of Compound 2

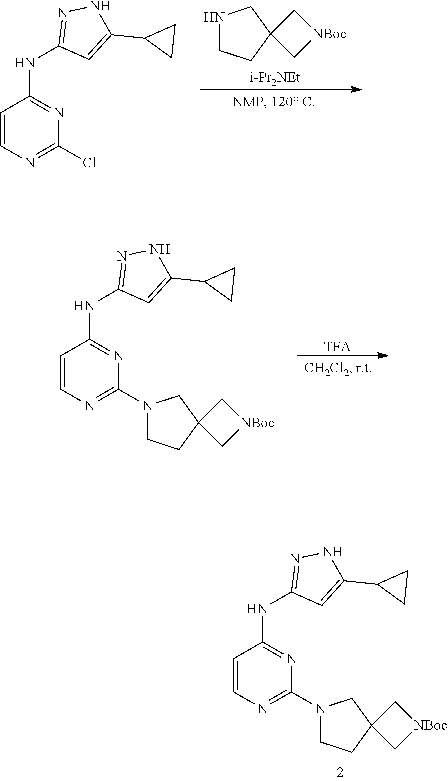

### tert-Butyl 6-(4-((5-Cyclopropyl-1H-pyrazol-3-yl)amino)pyrimidin-2-yl)-2,6-diazaspiro[3.4]octane-2-carboxylate

A solution of 2-chloro-N-(5-cyclopropyl-1H-pyrazol-3-yl)pyrimidin-4-amine (300 mg, 1.27 mmol), tert-butyl 2,6-diazaspiro[3.4]octane-2-carboxylate (405 mg, 1.91 mmol) and N,N-diisopropylethylamine (0.55 mL, 3.2 mmol) in NMP (3 mL) was heated to 120° C. in a sealed tube and stirred for 24 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (405 mg, 0.984 mmol, 77%)

¹H NMR (400 MHz, DMSO-d₆, 90° C.) δ 11.86 (s, 1H), 9.30 (s, 1H), 7.83 (d, J=5.8 Hz, 1H), 6.40-6.04 (m, 2H), 3.86-3.75 (m, 4H), 3.61 (s, 2H), 3.50 (t, J=6.7 Hz, 2H), 2.11 (t, J=6.8 Hz, 2H), 1.91-1.81 (m, 1H), 1.39 (s, 9H), 0.95-0.86 (m, 2H), 0.74-0.59 (m, 2H).

UPLC-MS (basic method, 2 min): Rt: 1.04 min, m/z: 412.4 [M+H]⁺.

### N-(5-cyclopropyl-1H-pyrazol-3-yl)-2-(2,6-diazaspiro[3.4]octan-6-yl)pyrimidin-4-amine

To a solution of tert-butyl 6-(4-((5-cyclopropyl-1H-pyrazol-3-yl)amino)pyrimidin-2-yl)-2,6-diazaspiro[3.4]octane-2-carboxylate (100 mg, 0.243 mmol) in CH₂Cl₂ (2 mL) was added trifluoroacetic acid (2.0 mL, 26 mmol) and the reaction mixture was stirred at ambient temperature for 1 hour, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The residue was purified by reversed-phase basic prepHPLC to afford the title compound as a solid (62 mg, 0.20 mmol, 82%).

¹H NMR (400 MHz, DMSO-d₆) δ 11.88 (s, 1H), 9.34 (s, 1H), 7.83 (d, J=5.7 Hz, 1H), 6.35-6.02 (m, 2H), 4.02 (s, 1H), 3.57 (s, 2H), 3.49-3.34 (m, 6H), 2.08 (t, J=6.8 Hz, 2H), 1.91-1.83 (m, 1H), 0.94-0.88 (m, 2H), 0.70-0.62 (m, 2H).

¹H NMR (400 MHz, methanol-d₄) δ 7.84 (d, J=5.8 Hz, 1H), 6.37-6.00 (m, 2H), 3.75-3.67 (m, 4H), 3.63-3.53 (m, 4H), 2.24 (t, J=6.9 Hz, 2H), 1.95-1.85 (m, 1H), 1.01-0.92 (m, 2H), 0.77-0.68 (m, 2H).

UPLC-MS (basic method, 4 min): Rt: 1.00 min, m/z: 312.3 [M+H]⁺.

### Example 3. Synthesis of Compound 3

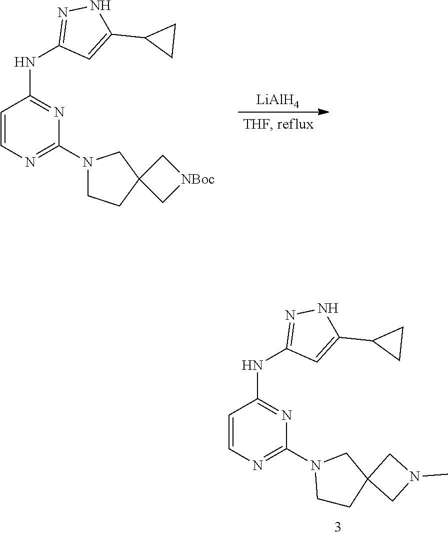 [SMILES: CC(C)(C)OC(=O)N1CC2(CCN(c3nccc(Nc4cc(C5CC5)[nH]n4)n3)C2)C1 ; "LiAlH4" ; CN1CC2(CCN(c3nccc(Nc4cc(C5CC5)[nH]n4)n3)C2)C1]

### N-(5-cyclopropyl-1H-pyrazol-3-yl)-2-(2-methyl-2,6-diazaspiro[3.4]octan-6-yl)pyrimidin-4-amine

To a solution of tert-butyl 6-(4-((5-cyclopropyl-1H-pyrazol-3-yl)amino)pyrimidin-2-yl)-2,6-diazaspiro[3.4]octane-2-carboxylate (150 mg, 0.365 mmol) in anhydrous THF (20 mL) was added a 2.4 M solution of lithium aluminum hydride in THF (1.5 mL, 3.6 mmol) dropwise at 0° C. The reaction mixture was heated to reflux and stirred for 2 hours. The reaction mixture was then cooled to 0° C., then quenched with water (150 μL), then a 15% w/v sodium hydroxide solution (150 μL), followed by water (450 μL). The reaction mixture was warmed to ambient temperature and stirred for 10 minutes, after which magnesium sulfate (ca. 1 g) was added and the reaction mixture was stirred for a further 10 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (64 mg, 0.20 mmol, 54%).

¹H NMR (400 MHz, DMSO-d₆) δ 11.87 (s, 1H), 9.32 (s, 1H), 7.82 (d, J=5.6 Hz, 1H), 6.39-6.03 (m, 2H), 3.54 (s, 2H), 3.45 (t, J=6.3 Hz, 2H), 3.12 (d, J=6.7 Hz, 2H), 3.06 (d, J=6.7 Hz, 2H), 2.22 (s, 3H), 2.05 (t, J=6.9 Hz, 2H), 1.92-1.80 (m, 1H), 0.94-0.88 (m, 2H), 0.70-0.61 (m, 2H).

¹H NMR (400 MHz, methanol-d₄) δ 7.83 (d, J=5.9 Hz, 1H), 6.36-5.96 (m, 2H), 3.65 (s, 2H), 3.57 (t, J=7.1 Hz, 2H), 3.37-3.26 (m, 4H), 2.38 (s, 3H), 2.19 (t, J=7.0 Hz, 2H), 1.96-1.85 (m, 1H), 1.01-0.94 (m, 2H), 0.77-0.69 (m, 2H).

UPLC-MS (basic method, 4 min): Rt: 1.14 min, m/z: 326.3 [M+H]⁺.

### Example 4. Synthesis of Compound 4

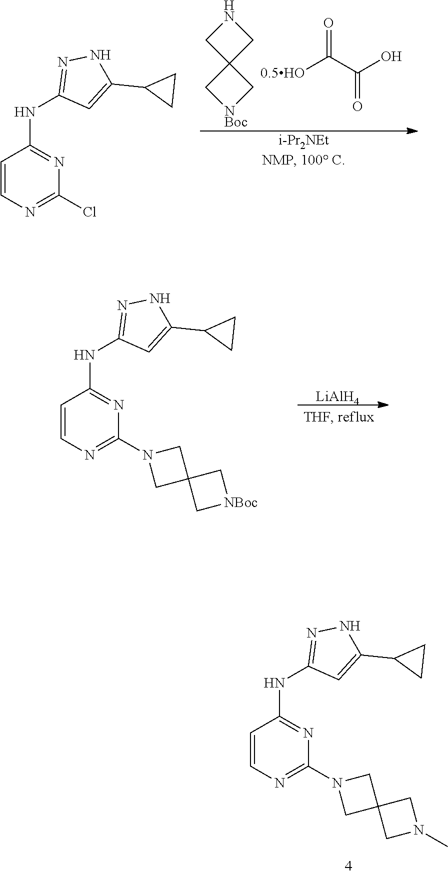

### tert-Butyl 6-(4-((5-Cyclopropyl-1H-pyrazol-3-yl)amino)pyrimidin-2-yl)-2,6-diazaspiro[3.3]heptane-2-carboxylate

A solution of 2-chloro-N-(5-cyclopropyl-1H-pyrazol-3-yl)pyrimidin-4-amine (300 mg, 1.27 mmol), tert-butyl 2,6-diazaspiro[3.3]heptane-2-carboxylate hemioxalate (465 mg, 1.91 mmol) and N,N-diisopropylethylamine (0.78 mL, 4.46 mmol) in NMP (5 mL) was heated to 100° C. and stirred for 16 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (300 mg, 0.611 mmol, 48%).

¹H NMR (400 MHz, methanol-d₄) δ 7.83 (d, J=6.0 Hz, 1H), 6.22 (s, 2H), 4.20 (s, 4H), 4.11 (s, 4H), 1.90 (m, 1H), 1.45 (s, 9H), 0.97 (d, J=8.0 Hz, 2H), 0.82-0.69 (m, 2H).

UPLC-MS (basic method, 2 min): Rt: 1.00 min, m/z: 398.4 [M+H]⁺.

### N-(5-Cyclopropyl-1H-pyrazol-3-yl)-2-(6-methyl-2,6-diazaspiro[3.3]heptan-2-yl)pyrimidin-4-amine

To a solution of tert-butyl 6-(4-((5-cyclopropyl-1H-pyrazol-3-yl)amino)pyrimidin-2-yl)-2,6-diazaspiro[3.3]heptane-2-carboxylate (149 mg, 0.375 mmol) in anhydrous THF (10 mL) was added a 2.4 M solution of lithium aluminum hydride in THF (1.6 mL, 3.8 mmol) dropwise at 0° C. The reaction mixture was heated to reflux and stirred for 2 hours. The reaction mixture was then cooled to 0° C., then quenched with water (150 μL), then a 15% w/v sodium hydroxide solution (150 μL), followed by water (450 μL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (16 mg, 21 μmol, 13%).

¹H NMR (400 MHz, DMSO-d₆) δ 11.87 (s, 1H), 9.43 (s, 1H), 7.82 (d, J=5.7 Hz, 1H), 6.32 (s, 1H), 6.14 (s, 1H), 4.00 (s, 4H), 3.25 (s, 4H), 2.17 (s, 3H), 1.98-1.64 (m, 1H), 1.05-0.80 (m, 2H), 0.75-0.56 (m, 2H).

UPLC-MS (basic method, 4 min): Rt: 1.08 min, m/z: 312.3 [M+H]⁺.

### Example 5. Synthesis of Compound 5

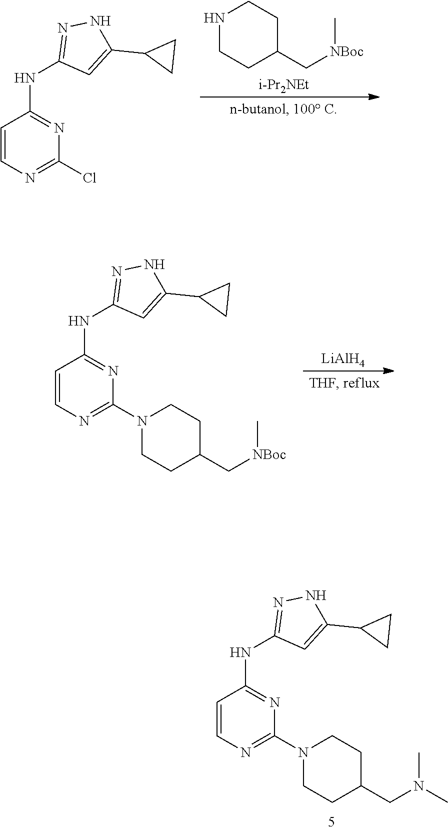

### tert-Butyl ((1-(4-((5-Cyclopropyl-1H-pyrazol-3-yl)amino)pyrimidin-2-yl)piperidin-4-yl)methyl)(methyl)carbamate

A solution of 2-chloro-N-(5-cyclopropyl-1H-pyrazol-3-yl)pyrimidin-4-amine (220 mg, 0.934 mmol), tert-butyl methyl(piperidin-4-ylmethyl)carbamate (533 mg, 2.33 mmol) and N,N-diisopropylethylamine (0.57 mL, 3.3 mmol) in n-butanol (10 mL) was heated to 100° C. and stirred for 24 hours. The reaction mixture was then concentrated to dryness under reduced pressure, then diluted with water (100 mL) and extracted with EtOAc (3×100 mL). The combined organic extracts were washed with water (2×100 mL), then a saturated aqueous brine solution (100 mL), then dried over Na₂SO₄ then concentrated to dryness under reduced pressure. The residue was purified by automated flash column chromatography, over silica gel, eluting with a gradient of CH₂Cl₂ to 1:9 2 M ammonia in MeOH:CH₂Cl₂ to afford the title compound as an oil (250 mg, 0.497 mmol, 53%)

¹H NMR (400 MHz, DMSO-d₆) δ 11.95 (s, 1H), 9.33 (s, 1H), 7.85 (d, J=5.7 Hz, 1H), 6.23 (s, 1H), 6.07 (s, 1H), 3.06 (d, J=7.1 Hz, 2H), 2.79 (d, J=9.5 Hz, 5H), 1.85 (s, 2H), 1.57 (s, 2H), 1.38 (s, 9H), 1.05 (d, J=12.2 Hz, 2H), 0.95-0.80 (m, 3H), 0.62 (d, J=5.2 Hz, 2H).

UPLC-MS (basic method, 2 min): Rt: 1.11 min, m/z: 428.4 [M+H]⁺.

### N-(5-Cyclopropyl-1H-pyrazol-3-yl)-2-(4-((dimethylamino)methyl)piperidin-1-yl)pyrimidin-4-amine

To a solution of tert-butyl ((1-(4-((5-cyclopropyl-1H-pyrazol-3-yl)amino)pyrimidin-2-yl)piperidin-4-yl)methyl)(methyl)carbamate (110 mg, 0.257 mmol) in anhydrous THE (10 mL) was added a 2.4 M solution of lithium aluminum hydride in THE (1.1 mL, 2.6 mmol) dropwise at 0° C. The reaction mixture was heated to reflux and stirred for 2.5 hours. The reaction mixture was then cooled to 0° C., then quenched with water (100 μL), then a 15% w/v sodium hydroxide solution (100 μL), followed by water (300 μL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (125 mg, 0.399 mmol, 27%).

¹H NMR (400 MHz, DMSO-d₆) δ 11.90 (s, 1H), 9.33 (s, 1H), 7.85 (d, J=5.6 Hz, 1H), 6.23 (s, 1H), 6.04 (s, 1H), 4.59 (d, J=13.1 Hz, 2H), 2.93-2.67 (m, 2H), 2.11 (s, 6H), 2.05 (d, J=6.8 Hz, 2H), 1.91-1.80 (m, 1H), 1.71 (d, J=11.3 Hz, 3H), 1.08-0.96 (m, 2H), 0.94-0.86 (m, 2H), 0.69-0.58 (m, 2H).

UPLC-MS (basic method, 4 min): Rt: 1.15 min, m/z: 342.1 [M+H]⁺.

### Example 6. Synthesis of Compound 6

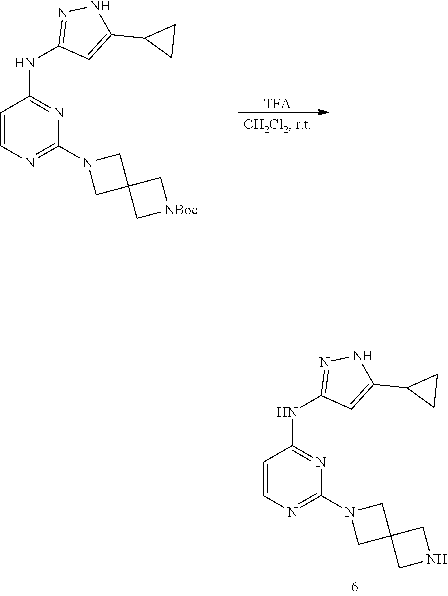 [SMILES: CC(C)(C)OC(=O)N1CC2(C1)CN(c1nccc(Nc3cc(C4CC4)[nH]n3)n1)C2 ; c1cc(Nc2cc(C3CC3)[nH]n2)nc(N2CC3(CNC3)C2)n1 ; "TFA"]

### N-(5-Cyclopropyl-1H-pyrazol-3-yl)-2-(2,6-diazaspiro[3.3]heptan-2-yl)pyrimidin-4-amine

To a solution of tert-butyl 6-(4-((5-cyclopropyl-1H-pyrazol-3-yl)amino)pyrimidin-2-yl)-2,6-diazaspiro[3.3]heptane-2-carboxylate (120 mg, 0.302 mmol) in CH₂Cl₂ (3 mL) was added trifluoroacetic acid (3.0 mL, 39 mmol) at 0° C. The reaction mixture was warmed to ambient temperature and stirred for 30 minutes. The reaction mixture was then concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase acidic prepHPLC, using TFA as the additive, followed by reversed-phase basic prepHPLC, using ammonia as the additive, to afford the title compound as a solid (36 mg, 0.12 mmol, 38%).

¹H NMR (400 MHz, DMSO-d₆) δ 11.92 (s, 1H), 9.42 (s, 1H), 7.82 (d, J=5.8 Hz, 1H), 6.31 (s, 1H), 6.14 (s, 1H), 4.02 (s, 4H), 3.59 (s, 4H), 2.02-1.66 (m, 1H), 0.91 (d, J=7.9 Hz, 2H), 0.66 (d, J=5.2 Hz, 2H).

UPLC-MS (basic method, 4 min): Rt: 0.89 min, m/z: 298.3 [M+H]⁺.

### Example 7. Synthesis of Compound 7

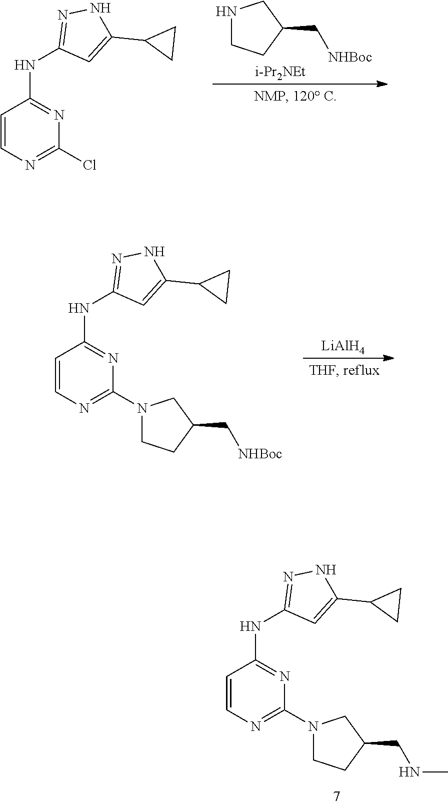 [SMILES: "i-Pr2NEt" ; Clc1nccc(Nc2cc(C3CC3)[nH]n2)n1 ; CC(C)(C)OC(=O)NC[C@H]1CCNC1 ; CC(C)(C)OC(=O)NC[C@H]1CCN(c2nccc(Nc3cc(C4CC4)[nH]n3)n2)C1 ; "LiAlH4" ; CNC[C@H]1CCN(c2nccc(Nc3cc(C4CC4)[nH]n3)n2)C1]

### tert-Butyl (R)-((1-(4-((5-Cyclopropyl-1H-pyrazol-3-yl)amino)pyrimidin-2-yl)pyrrolidin-3-yl)methyl)carbamate

A solution of 2-chloro-N-(5-cyclopropyl-1H-pyrazol-3-yl)pyrimidin-4-amine (600 mg, 2.55 mmol), (S)-tert-butyl (pyrrolidin-3-ylmethyl)carbamate (765 mg, 3.82 mmol) and N,N-diisopropylethylamine (1.1 mL, 6.4 mmol) in NMP (6 mL) was heated to 120° C. in a sealed tube and stirred for 21 hours. The reaction mixture was then cooled to ambient temperature, then added dropwise to stirring ice water (250 mL), resulting in the formation of a precipitate. The precipitate was collected by filtration, washed with water (2×10 mL) then dried under reduced pressure to afford the title compound as a solid (810 mg, 2.02 mmol, 79%).

¹H NMR (400 MHz, DMSO-d₆) δ 11.86 (s, 1H), 9.31 (s, 1H), 7.82 (d, J=5.7 Hz, 1H), 6.99 (s, 1H), 6.37-6.01 (m, 2H), 3.61-3.52 (m, 2H), 3.46-3.37 (m, 1H), 3.17-3.12 (m, 1H), 3.03-2.95 (m, 2H), 2.44-2.30 (m, 1H), 2.02-1.91 (m, 1H), 1.89-1.82 (m, 1H), 1.70-1.61 (m, 1H), 1.38 (s, 9H), 0.97-0.86 (m, 2H), 0.69-0.63 (m, 2H).

UPLC-MS (basic method, 2 min): Rt: 1.00 min, m/z: 400.4 [M+H]⁺.

### (R)—N-(5-Cyclopropyl-1H-pyrazol-3-yl)-2-(3-((methylamino)methyl)pyrrolidin-1-yl)pyrimidin-4-amine

To a solution of tert-butyl tert-butyl (R)-((1-(4-((5-cyclopropyl-1H-pyrazol-3-yl)amino)pyrimidin-2-yl)pyrrolidin-3-yl)methyl)carbamate (600 mg, 1.50 mmol) in anhydrous THF (20 mL) was added a 2.4 M solution of lithium aluminum hydride in THE (6.3 mL, 15 mmol) dropwise at 0° C. The reaction mixture was heated to reflux and stirred for 4 hours. The reaction mixture was then cooled to 0° C., then quenched with water (0.65 mL), then a 15% w/v sodium hydroxide solution (0.65 mL), followed by water (1.95 mL). The reaction mixture was warmed to ambient temperature and stirred for 15 minutes, after which magnesium sulfate (ca. 5 g) was added and the reaction mixture was stirred for a further 15 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (125 mg, 0.399 mmol, 27%).

¹H NMR (400 MHz, methanol-d₄) δ 7.84 (d, J=5.9 Hz, 1H), 6.21-6.03 (m, 2H), 3.77 (dd, J=10.4, 7.7 Hz, 1H), 3.73-3.65 (m, 1H), 3.56-3.45 (m, 1H), 3.19 (t, J=9.3 Hz, 1H), 2.72-2.59 (m, 2H), 2.51 (dq, J=14.8, 7.3 Hz, 1H), 2.41 (s, 3H), 2.25-2.13 (m, 1H), 1.89 (tt, J=8.4, 5.1 Hz, 1H), 1.74 (dq, J=12.3, 8.5 Hz, 1H), 1.01-0.90 (m, 2H), 0.75-0.67 (m, 2H).

UPLC-MS (basic method, 4 min): Rt: 1.08 min, m/z: 314.1 [M+H]⁺.

### Example 8. Synthesis of Compound 8

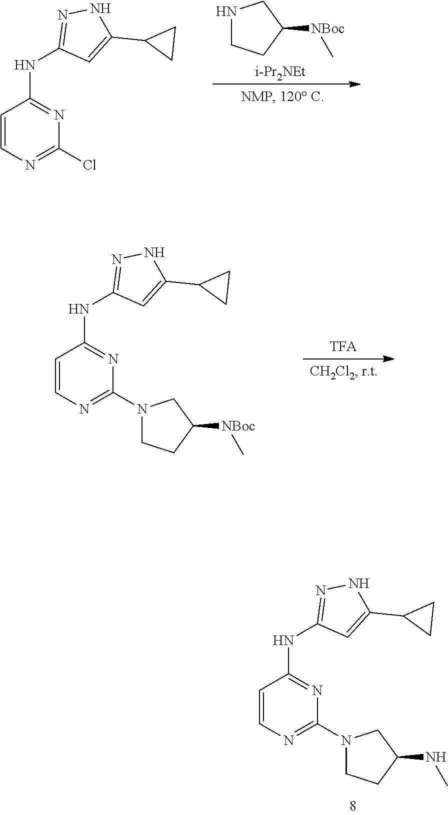 [SMILES: "i-Pr2NEt" ; Clc1nccc(Nc2cc(C3CC3)[nH]n2)n1 ; CN(C(=O)OC(C)(C)C)[C@H]1CCNC1 ; CN(C(=O)OC(C)(C)C)[C@H]1CCN(c2nccc(Nc3cc(C4CC4)[nH]n3)n2)C1 ; CN[C@H]1CCN(c2nccc(Nc3cc(C4CC4)[nH]n3)n2)C1 ; "TFA"]

### tert-Butyl (S)-(1-(4-((5-Cyclopropyl-1H-pyrazol-3-yl)amino)pyrimidin-2-yl)pyrrolidin-3-yl)(methyl)carbamate

A solution of 2-chloro-N-(5-cyclopropyl-1H-pyrazol-3-yl)pyrimidin-4-amine (300 mg, 1.27 mmol), (S)-tert-butyl methyl(pyrrolidin-3-yl)carbamate (382 mg, 1.91 mmol) and N,N-diisopropylethylamine (0.55 mL, 3.2 mmol) in NMP (3 mL) was heated to 120° C. in a sealed tube and stirred for 20 hours. The reaction mixture was then cooled to ambient temperature, then added dropwise to stirring ice water (250 mL), resulting in the formation of a precipitate. The precipitate was collected by filtration, washed with water (2×10 mL) then dried under reduced pressure to afford the title compound as a solid (389 mg, 0.945 mmol, 74%)

¹H NMR (400 MHz, DMSO-d₆) δ 11.89 (s, 1H), 9.36 (s, 1H), 7.84 (d, J=5.8 Hz, 1H), 6.23 (s, 2H), 4.64 (s, 1H), 3.72-3.62 (m, 2H), 3.48-3.32 (m, 2H), 2.75 (s, 3H), 2.12-1.98 (m, 2H), 1.88-1.80 (m, 1H), 1.42 (s, 9H), 0.92-0.85 (m, 2H), 0.68-0.59 (m, 2H).

UPLC-MS (basic method, 2 min): Rt: 1.05 min, m/z: 400.4 [M+H]⁺.

### (S)—N-(5-cyclopropyl-1H-pyrazol-3-yl)-2-(3-(methylamino)pyrrolidin-1-yl)pyrimidin-4-amine

To a solution of tert-butyl (S)-(1-(4-((5-cyclopropyl-1H-pyrazol-3-yl)amino)pyrimidin-2-yl)pyrrolidin-3-yl)(methyl)carbamate (100 mg, 0.250 mmol) in CH₂Cl₂ (2 mL) was added trifluoracetic acid (2.0 mL, 26 mmol) and the reaction mixture was stirred at ambient temperature for 1 hour, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase acidic prepHPLC, using TFA as the additive, followed by reversed-phase basic prepHPLC, using ammonia as the additive, to afford the title compound as a solid (37 mg, 0.12 mmol, 49%).

¹H NMR (400 MHz, DMSO-d₆) δ 11.88 (s, 1H), 9.32 (s, 1H), 7.82 (d, J=5.7 Hz, 1H), 6.40-6.00 (m, 2H), 3.66-3.49 (m, 2H), 3.48-3.39 (m, 1H), 3.25 (d, J=4.7 Hz, 1H), 3.21-3.12 (m, 1H), 2.29 (d, J=4.9 Hz, 3H), 2.02 (dq, J=12.5, 6.5 Hz, 1H), 1.91-1.70 (m, 3H), 0.95-0.86 (m, 2H), 0.72-0.60 (m, 2H).

¹H NMR (400 MHz, methanol-d₄) δ 7.83 (d, J=6.0 Hz, 1H), 6.36-6.00 (m, 2H), 3.79 (dd, J=10.4, 5.6 Hz, 1H), 3.75-3.66 (m, 1H), 3.61-3.51 (m, 1H), 3.42-3.33 (m, 2H), 2.43 (s, 3H), 2.30-2.17 (m, 1H), 1.96-1.83 (m, 2H), 1.01-0.92 (m, 2H), 0.76-0.67 (m, 2H).

UPLC-MS (basic method, 4 min): Rt: 1.02 min, m/z: 300.1 [M+H]⁺.

### Example 9. Synthesis of Compound 9

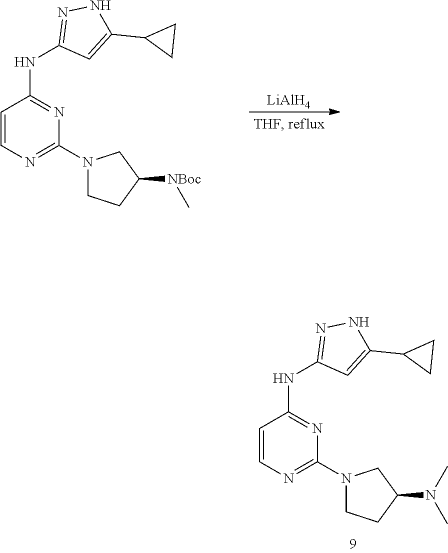 [SMILES: CN(C(=O)OC(C)(C)C)[C@H]1CCN(c2nccc(Nc3cc(C4CC4)[nH]n3)n2)C1 ; "LiAlH4" ; CN(C)[C@H]1CCN(c2nccc(Nc3cc(C4CC4)[nH]n3)n2)C1]

### (S)—N-(5-Cyclopropyl-1H-pyrazol-3-yl)-2-(3-(dimethylamino)pyrrolidin-1-yl)pyrimidin-4-amine

To a solution of tert-butyl (S)-(1-(4-((5-cyclopropyl-1H-pyrazol-3-yl)amino)pyrimidin-2-yl)pyrrolidin-3-yl)(methyl)carbamate (150 mg, 0.375 mmol) in anhydrous THF (10 mL) was added a 2.4 M solution of lithium aluminum hydride in THF (1.6 mL, 3.8 mmol) dropwise at 0° C. The reaction mixture was heated to reflux and stirred for 6 hours. The reaction mixture was then cooled to 0° C., then quenched with water (150 μL), then a 15% w/v sodium hydroxide solution (150 μL), followed by water (450 μL). The reaction mixture was warmed to ambient temperature and stirred for 10 minutes, after which magnesium sulfate (ca. 1 g) was added and the reaction mixture was stirred for a further 10 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (64 mg, 0.20 mmol, 54%).

¹H NMR (400 MHz, DMSO-d₆) δ 11.90 (s, 1H), 9.37 (s, 1H), 7.84 (d, J=5.7 Hz, 1H), 6.21 (s, 2H), 3.77 (dd, J=10.6, 7.1 Hz, 1H), 3.67 (t, J=9.8 Hz, 1H), 3.45-3.34 (m, 1H), 3.14 (t, J=9.5 Hz, 1H), 2.78-2.68 (m, 1H), 2.21 (s, 6H), 2.18-2.07 (m, 1H), 1.85 (tt, J=8.4, 5.1 Hz, 1H), 1.81-1.70 (m, 1H), 0.96-0.83 (m, 2H), 0.70-0.59 (m, 2H).

¹H NMR (400 MHz, chloroform-d) δ 8.01 (d, J=5.7 Hz, 1H), 6.89 (s, 1H), 6.04 (s, 1H), 5.91 (s, 1H), 3.93 (dd, J=10.4, 7.1 Hz, 1H), 3.82 (ddd, J=10.9, 8.8, 2.0 Hz, 1H), 3.53 (td, J=10.6, 6.9 Hz, 1H), 3.33 (t, J=9.6 Hz, 1H), 2.84-2.72 (m, 1H), 2.32 (s, 6H), 2.27-2.16 (m, 1H), 1.97-1.80 (m, 2H), 0.99-0.88 (m, 2H), 0.77-0.68 (m, 2H).

UPLC-MS (basic method, 4 min): Rt: 1.22 min, m/z: 314.3 [M+H]⁺.

### Example 10. Synthesis of Compound 10

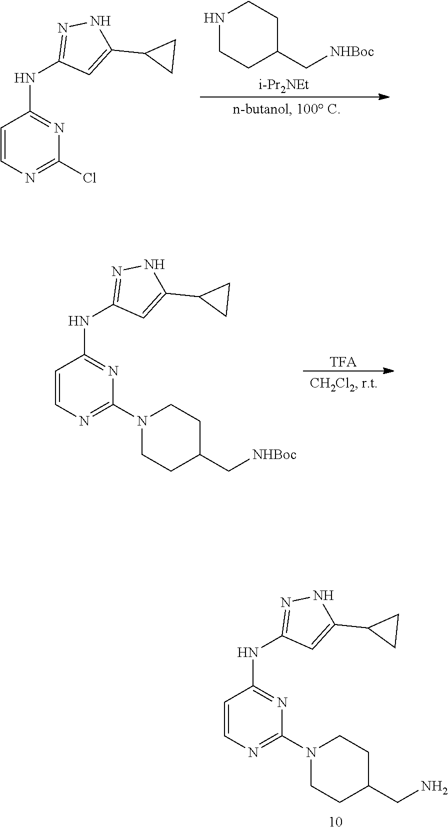

### tert-Butyl ((1-(4-((5-Cyclopropyl-1H-pyrazol-3-yl)amino)pyrimidin-2-yl)piperidin-4-yl)methyl)carbamate

A solution of 2-chloro-N-(5-cyclopropyl-1H-pyrazol-3-yl)pyrimidin-4-amine (120 mg, 0.509 mmol), tert-butyl 4-piperidinylmethylcarbamate (273 mg, 1.27 mmol) and N,N-diisopropylethylamine (0.31 mL, 1.8 mmol) in n-butanol (5 mL) was heated to 100° C. and stirred for 24 hours. The reaction mixture was then concentrated to dryness under reduced pressure, the residue was dissolved in CH₂Cl₂ (30 mL) and the organic layer was washed with water (2×30 mL), then a saturated aqueous brine solution (30 mL). The organic layer was then dried over Na₂SO₄ then concentrated to dryness under reduced pressure. The residue was purified by automated flash column chromatography, over silica gel, eluting with a gradient of CH₂Cl₂ to 1:9 2 M NH₃ in MeOH:CH₂Cl₂ to afford the title compound as a solid (115 mg, 0.278 mmol, 54%)

¹H NMR (400 MHz, methanol-d₄) δ 7.84 (d, J=5.7 Hz, 1H), 6.14 (s, 2H), 4.63 (d, J=13.2 Hz, 2H), 3.35 (s, 1H), 2.95 (d, J=6.2 Hz, 2H), 2.87 (t, J=12.9 Hz, 2H), 1.93-1.82 (m, 1H), 1.74 (d, J=12.0 Hz, 2H), 1.44 (s, 9H), 1.24-1.07 (m, 2H), 0.97 (d, J=8.2 Hz, 2H), 0.79-0.62 (m, 2H).

UPLC-MS (basic method, 2 min): Rt: 1.04 min, m/z: 414.4 [M+H]⁺.

### 2-(4-(aminomethyl)piperidin-1-yl)-N-(5-cyclopropyl-1H-pyrazol-3-yl)pyrimidin-4-amine

To a solution of tert-butyl ((1-(4-((5-cyclopropyl-1H-pyrazol-3-yl)amino)pyrimidin-2-yl)piperidin-4-yl)methyl)carbamate (115 mg, 0. 278 mmol) in CH₂Cl₂ (3 mL) was added trifluoracetic acid (3.0 mL, 26 mmol) at 0° C. The reaction mixture was warmed to ambient temperature and stirred for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC, to afford the title compound as a solid (66 mg, 0.21 mmol, 74%).

¹H NMR (400 MHz, DMSO-d₆) δ 11.92 (s, 1H), 9.30 (s, 1H), 7.86 (d, J=5.6 Hz, 1H), 6.23 (s, 1H), 6.05 (s, 1H), 4.62 (d, J=13.0 Hz, 2H), 2.88-2.73 (m, 2H), 2.58 (d, J=6.4 Hz, 1H), 1.92-1.82 (m, 2H), 1.81-1.56 (m, 3H), 1.18-0.99 (m, 2H), 0.96-0.86 (m, 2H), 0.68-0.47 (m, 2H).

UPLC-MS (basic method, 4 min): Rt: 1.04 min, m/z: 314.3 [M+H]⁺.

### Example 11. Synthesis of Compound 11

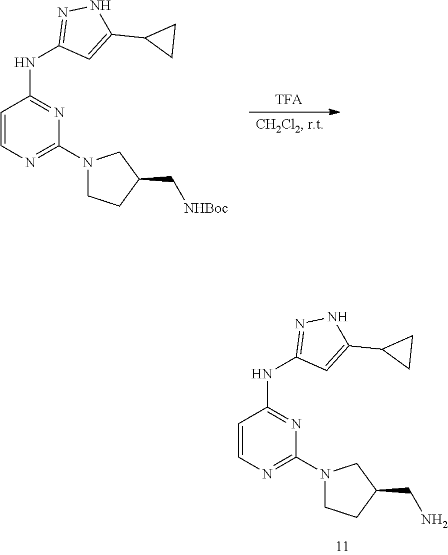 [SMILES: CC(C)(C)OC(=O)NC[C@H]1CCN(c2nccc(Nc3cc(C4CC4)[nH]n3)n2)C1 ; NC[C@H]1CCN(c2nccc(Nc3cc(C4CC4)[nH]n3)n2)C1 ; "TFA"]

### (R)-2-(3-(Aminomethyl)pyrrolidin-1-yl)-N-(5-cyclopropyl-1H-pyrazol-3-yl)pyrimidin-4-amine

To a solution of tert-butyl (R)-((1-(4-((5-cyclopropyl-1H-pyrazol-3-yl)amino)pyrimidin-2-yl)pyrrolidin-3-yl)methyl)carbamate (100 mg, 0.250 mmol) in CH₂Cl₂ (2 mL) was added trifluoroacetic acid (2.0 mL, 26 mmol) and the reaction mixture was stirred at ambient temperature for 1 hour, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase acidic prepHPLC, using TFA as the additive, followed by reversed-phase basic prepHPLC, using ammonia as the additive, to afford the title compound as a solid (37 mg, 0.12 mmol, 49%).

¹H NMR (400 MHz, DMSO-d₆) δ 11.87 (s, 1H), 9.31 (s, 1H), 7.82 (d, J=5.7 Hz, 1H), 6.44-5.95 (m, 2H), 3.66-3.50 (m, 2H), 3.46-3.36 (m, 1H), 3.21-3.10 (m, 1H), 2.59 (t, J=6.1 Hz, 2H), 2.28-2.17 (m, 1H), 2.07-1.94 (m, 1H), 1.90-1.79 (m, 1H), 1.67 (q, J=9.4 Hz, 1H), 0.95-0.86 (m, 2H), 0.69-0.60 (m, 2H).

¹H NMR (400 MHz, methanol-d₄) δ 7.84 (d, J=5.9 Hz, 1H), 6.11 (s, 2H), 3.80-3.64 (m, 2H), 3.52 (q, J=8.7 Hz, 1H), 3.21 (t, J=9.4 Hz, 1H), 2.82-2.67 (m, 2H), 2.41 (p, J=7.1 Hz, 1H), 2.22-2.11 (m, 1H), 1.95-1.83 (m, 1H), 1.81-1.67 (m, 1H), 0.99-0.91 (m, 2H), 0.74-0.67 (m, 2H).

UPLC-MS (basic method, 4 min): Rt: 1.03 min, m/z: 300.1 [M+H]⁺.

### Example 12. Synthesis of Compound 12

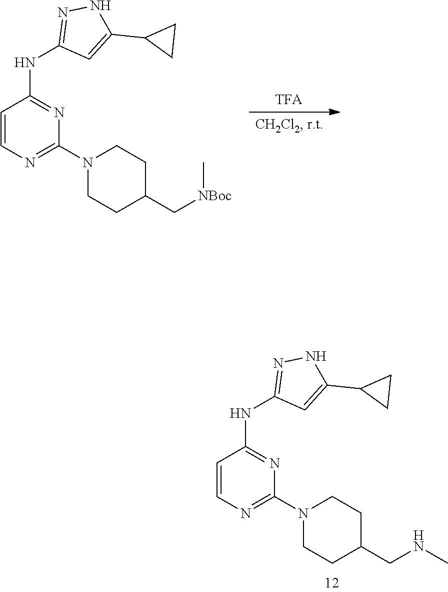 [SMILES: CN(CC1CCN(c2nccc(Nc3cc(C4CC4)[nH]n3)n2)CC1)C(=O)OC(C)(C)C ; CNCC1CCN(c2nccc(Nc3cc(C4CC4)[nH]n3)n2)CC1 ; "TFA"]

### N-(5-Cyclopropyl-1H-pyrazol-3-yl)-2-(4-((methylamino)methyl)piperidin-1-yl)pyrimidin-4-amine

To a solution of tert-butyl ((1-(4-((5-Cyclopropyl-1H-pyrazol-3-yl)amino)pyrimidin-2-yl)piperidin-4-yl)methyl)(methyl)carbamate (111 mg, 0.258 mmol) in CH₂Cl₂ (3 mL) was added trifluoracetic acid (3.0 mL, 26 mmol) at 0° C. The reaction mixture was warmed to ambient temperature and stirred for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC, to afford the title compound as a solid (78 mg, 0.24 mmol, 92%).

¹H NMR (400 MHz, DMSO-d₆) δ 11.92 (s, 1H), 9.30 (s, 1H), 7.85 (d, J=5.6 Hz, 1H), 6.22 (s, 1H), 6.04 (s, 1H), 4.60 (d, J=12.9 Hz, 2H), 2.91-2.71 (m, 3H), 2.33 (d, J=6.3 Hz, 2H), 2.26 (s, 3H), 1.93-1.77 (m, 1H), 1.76-1.54 (m, 3H), 1.10-0.98 (m, 2H), 0.95-0.87 (m, 2H), 0.69-0.55 (m, 2H).

UPLC-MS (basic method, 4 min): Rt: 1.69 min, m/z: 328.3 [M+H]⁺.

### Example 13. Synthesis of Compound 13

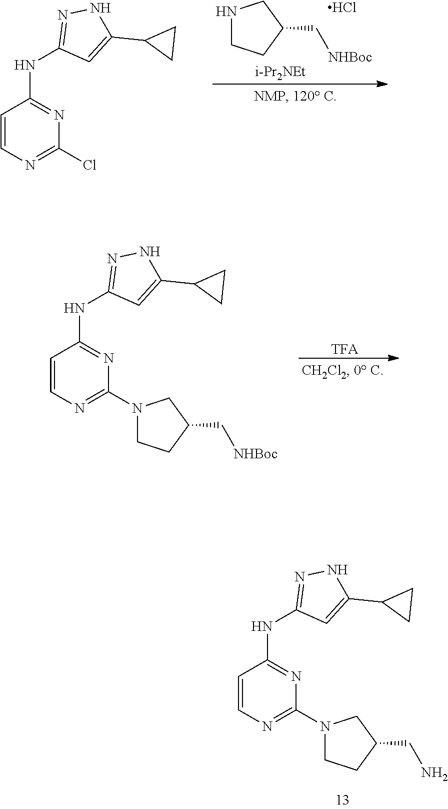

### tert-Butyl (S)-(1-(4-((5-Cyclopropyl-1H-pyrazol-3-yl)amino)pyrimidin-2-yl)pyrrolidin-3-yl)(methyl)carbamate

A solution of 2-chloro-N-(5-cyclopropyl-1H-pyrazol-3-yl)pyrimidin-4-amine (670 mg, 2.84 mmol), tert-butyl N-[(3R)-pyrrolidin-3-ylmethyl]carbamate hydrochloride (1.01 g, 4.26 mmol) and N,N-diisopropylethylamine (1.7 mL, 10 mmol) in NMP (8 mL) was heated to 120° C. and stirred for 21 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude material was purified by automated flash column chromatography, over silica gel, eluting with a gradient of CH₂Cl₂ to 1:9 2 M NH₃ in MeOH:CH₂Cl₂ to afford the title compound as an oil (800 mg, 1.92 mmol, 68%)

¹H NMR (400 MHz, DMSO-d₆) δ 11.86 (s, 1H), 9.31 (s, 1H), 7.82 (d, J=5.7 Hz, 1H), 6.99 (d, J=6.5 Hz, 1H), 6.20 (s, 2H), 4.15-4.00 (m, 2H), 3.67-3.49 (m, 2H), 2.99 (t, J=6.6 Hz, 2H), 2.41-2.33 (m, 1H), 2.05-1.92 (m, 1H), 1.88-1.78 (m, 1H), 1.75-1.59 (m, 1H), 1.38 (s, 9H), 0.90 (d, J=8.2 Hz, 2H), 0.72-0.60 (m, 2H).

UPLC-MS (basic method, 2 min): Rt: 1.00 min, m/z: 400.4 [M+H]⁺.

### (S)—N-(5-cyclopropyl-1H-pyrazol-3-yl)-2-(3-(methylamino)pyrrolidin-1-yl)pyrimidin-4-amine

To a solution of tert-butyl (S)-(1-(4-((5-cyclopropyl-1H-pyrazol-3-yl)amino)pyrimidin-2-yl)pyrrolidin-3-yl)(methyl)carbamate (120 mg, 0.300 mmol) in CH₂Cl₂ (4 mL) was added trifuoroacetic acid (4.0 mL, 52 mmol) at 0° C. The reaction mixture was held at 0° C. and stirred for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC, to afford the title compound as a solid (30 mg, 94 μmol, 31%).

¹H NMR (400 MHz, methanol-d₄) δ 7.84 (d, J=5.9 Hz, 1H), 6.36-6.00 (m, 2H), 3.83-3.65 (m, 2H), 3.56-3.41 (m, 1H), 3.22 (t, J=9.4 Hz, 1H), 2.80 (m, 2H), 2.50-2.35 (m, 1H), 2.19 (q, J=5.8, 5.2 Hz, 1H), 1.98-1.83 (m, 1H), 1.80-1.65 (m, 1H), 0.96 (d, J=8.1 Hz, 2H), 0.72 (d, J=3.5, 2.0 Hz, 2H).

UPLC-MS (basic method, 4 min): Rt: 1.04 min, m/z: 300.1 [M+H]⁺.

### Example 14. Synthesis of Compound 14

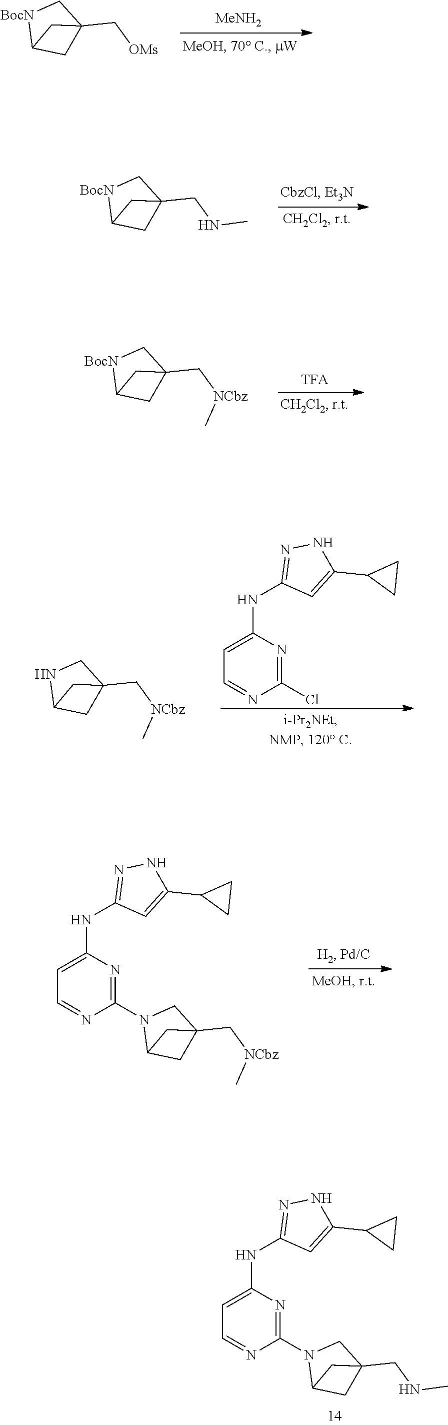

### tert-Butyl 4-((Methylamino)methyl)-2-azabicyclo[2.1.1]hexane-2-carboxylate

A solution of 2-methyl-2-propanyl 4-{[(methylsulfonyl)oxy]methyl}-2-azabicyclo[2.1.1]hexane-2-carboxylate (1.48 g, 5.08 mmol) in a 2.0 M solution of methylamine in MeOH (68 mL) was heated to 70° C. in a microwave reactor and stirred for 2 hours. The reaction mixture was concentrated to dryness under reduced pressure then the residue was diluted with a 0.5 M aqueous solution of NaOH (100 mL), then extracted with EtOAc (3×100 mL). The combined organic extracts were dried over Na₂SO₄, then concentrated to dryness under reduced pressure, to afford the title compound as an oil (820 mg, 3.62 mmol, 71%).

¹H NMR (400 MHz, chloroform-d) δ 4.34 (s, 1H), 3.23 (s, 2H), 2.83 (s, 2H), 2.47 (s, 3H), 1.78-1.70 (m, 2H), 1.54-1.38 (m, 11H).

UPLC-MS (basic method, 2 min): Rt: 0.92 min, m/z=227.1 [M+H]⁺.

### tert-Butyl 4-((((Benzyloxy)carbonyl)(methyl)amino)methyl)-2-azabicyclo[2.1.1]hexane-2-carboxylate

To a solution of tert-butyl 4-((methylamino)methyl)-2-azabicyclo[2.1.1]hexane-2-carboxylate (815 mg, 3.60 mmol) and triethylamine (753 μL, 5.40 mmol) in CH₂Cl₂ (10 mL) was added benzyl chloroformate (565 μL, 3.96 mmol) and the reaction mixture was stirred at ambient temperature for 3 hours. The reaction mixture was then diluted with water (50 mL) then extracted with CH₂Cl₂ (3×50 mL). The combined organic extracts were washed with a saturated aqueous brine solution (50 mL) then concentrated to dryness under reduced pressure. The crude material was purified by automated flash column chromatography, over silica gel, eluting with a gradient of 1:4 EtOAc:iso-hexane to 4:6 EtOAc:iso-hexane to afford the title compound as an oil (1.16 g, 3.12 mmol, 87%).

¹H NMR (400 MHz, chloroform-d) δ 7.39-7.28 (m, 5H), 5.14-5.08 (m, 2H), 4.29 (s, 1H), 3.57-3.49 (m, 2H), 3.29-3.11 (m, 2H), 2.99-2.93 (m, 3H), 1.78-1.66 (m, 2H), 1.46 (s, 11H).

UPLC-MS (basic method, 2 min): Rt: 1.26 min, m/z=361.1 [M+H]⁺.

### Benzyl ((2-azabicyclo[2.1.1]hexan-4-yl)methyl)(methyl)carbamate

To a solution of tert-butyl 4-((((benzyloxy)carbonyl)(methyl)amino)methyl)-2-azabicyclo[2.1.1]hexane-2-carboxylate (1.15 g, 3.19 mmol) in CH₂Cl₂ (10 mL) was added trifluoroacetic acid (5.0 mL, 65 mmol) and the reaction mixture was stirred at room temperature for 30 minutes. The crude residue was loaded onto an SCX cartridge, the cartridge was then washed with methanol, then the compound was eluted with 2 M NH₃ in methanol, and the eluent was concentrated to dryness under reduced pressure to afford the title compound as oil (760 mg, 2.92 mmol, 92%).

¹H NMR (400 MHz, chloroform-d) δ 7.39-7.29 (m, 5H), 5.15-5.08 (m, 2H), 3.60-3.51 (m, 2H), 2.99-2.93 (m, 3H), 2.84-2.72 (m, 2H), 1.65-1.55 (m, 2H), 1.36-1.23 (m, 2H).

UPLC-MS (basic method, 2 min): Rt: 0.86 min, m/z=261.1 [M+H]⁺.

### Benzyl ((2-(4-((5-Cyclopropyl-1H-pyrazol-3-yl)amino)pyrimidin-2-yl)-2-azabicyclo[2.1.1]hexan-4-yl)methyl)(methyl)carbamate

A solution of 2-chloro-N-(5-cyclopropyl-1H-pyrazol-3-yl)pyrimidin-4-amine (570 mg, 2.42 mmol), benzyl ((2-azabicyclo[2.1.1]hexan-4-yl)methyl)(methyl)carbamate (756 mg, 2.90 mmol) and N,N-diisopropylethylamine (0.93 mL, 5.3 mmol) in NMP (6 mL) was heated to 120° C. in a sealed tube and stirred for 18 hours. The reaction mixture was cooled to ambient temperature, then added dropwise to stirring ice water (200 mL), resulting in the formation of a precipitate. The precipitate was collected by filtration, washed with water (2×10 mL) then dried under reduced pressure to afford the title compound as a solid (970 mg, 2.11 mmol, 87%).

UPLC-MS (basic method, 2 min): Rt: 1.07 min, m/z=460.4 [M+H]⁺.

### N-(5-cyclopropyl-1H-pyrazol-3-yl)-2-(4-((methylamino)methyl)-2-azabicyclo[2.1.1]hexan-2-yl)pyrimidin-4-amine

To a solution of benzyl ((2-(4-((5-cyclopropyl-1H-pyrazol-3-yl)amino)pyrimidin-2-yl)-2-azabicyclo[2.1.1]hexan-4-yl)methyl)(methyl)carbamate (220 mg, 0.479 mmol) in Methanol (5 mL) was added a suspension of 10% w/w palladium on activated carbon (51 mg, 48 mol) in water (0.1 mL) under a flow of nitrogen. The reaction vessel was then placed under vacuum and backfilled with nitrogen three times. The reaction vessel was then placed under vacuum, then back-filled with hydrogen a total of three times. The reaction mixture was then stirred at room temperature for 3 hours. The reaction mixture was then filtered through a pad of celite, and the filter cake was washed with EtOAc (10 mL). The eluent was concentrated to dryness, under reduced pressure. The residue was purified by reversed-phase basic prepHPLC to afford the title compound as a solid (90 mg, 0.277 mmol, 58%).

¹H NMR (400 MHz, DMSO-d₆) δ 11.89 (s, 1H), 9.31 (s, 1H), 7.83 (d, J=5.7 Hz, 1H), 6.35-5.97 (m, 2H), 4.68 (s, 1H), 3.32 (s, 2H), 2.75 (s, 2H), 2.32 (s, 3H), 1.86 (tt, J=8.5, 5.1 Hz, 1H), 1.82-1.76 (m, 2H), 1.42-1.31 (m, 2H), 0.95-0.86 (m, 2H), 0.69-0.62 (m, 2H).

¹H NMR (400 MHz, methanol-d₄) δ 7.83 (d, J=6.0 Hz, 1H), 6.27-5.92 (m, 1H), 4.78 (s, 1H), 3.44 (s, 2H), 2.89 (s, 2H), 2.44 (s, 3H), 1.97-1.84 (m, 3H), 1.58-1.47 (m, 1H), 1.02-0.92 (m, 2H), 0.77-0.68 (m, 2H).

UPLC-MS (basic method, 2 min): Rt: 1.15 min, m/z=326.0 [M+H]⁺.

### Example 15. Synthesis of Compound 15

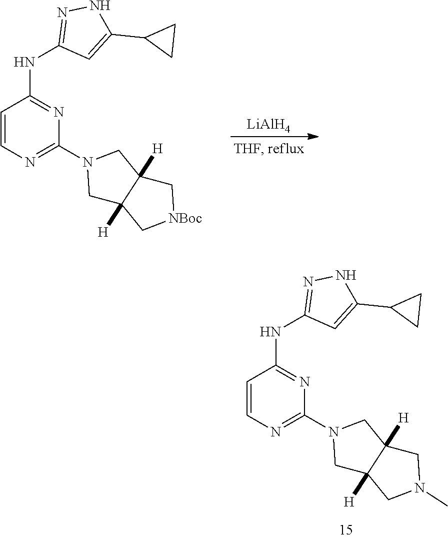 [SMILES: CC(C)(C)OC(=O)N1C[C@@H]2CN(c3nccc(Nc4cc(C5CC5)[nH]n4)n3)C[C@@H]2C1 ; "LiAlH4" ; CN1C[C@@H]2CN(c3nccc(Nc4cc(C5CC5)[nH]n4)n3)C[C@@H]2C1]

### 2-[rac-(3aR,6aS)-2-methyl-1,3,3a,4,6,6a-hexahydropyrrolo[3,4-c]pyrrol-5-yl]-N-(5-cyclopropyl-1H-pyrazol-3-yl)pyrimidin-4-amine

To a solution of tert-butyl rac-(3aS,6aR)-2-[4-[(5-cyclopropyl-1H-pyrazol-3-yl)amino]pyrimidin-2-yl]-1,3,3a,4,6,6a hexahydropyrrolo[3,4-c]pyrrole-5-carboxylate (200 mg, 0.486 mmol) in anhydrous THE (10 mL) was added a 2.4 M solution of lithium aluminum hydride in THE (2.0 mL, 4.9 mmol) dropwise at 0° C. The reaction mixture was heated to reflux and stirred for 2 hours. The reaction mixture was then cooled to 0° C., then quenched with water (1.8 mL), then a 15% w/v sodium hydroxide solution (1.8 mL), followed by water (5.2 mL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (13 mg, 39 μmol, 8%).

¹H NMR (400 MHz, chloroform-d) δ 8.01 (d, J=1.2 Hz, 1H), 6.82 (s, 1H), 6.02 (s, 1H), 5.79 (s, 1H), 3.87-3.72 (m, 2H), 3.56 (dd, J=11.2, 3.0 Hz, 2H), 2.97 (s, 2H), 2.71 (t, J=7.7 Hz, 2H), 2.48 (d, J=9.5 Hz, 2H), 2.33 (s, 3H), 1.91-1.81 (m, 1H), 0.95 (dd, J=8.4, 2.2 Hz, 2H), 0.74 (dd, J=5.0, 2.0 Hz, 2H).

UPLC-MS (basic method, 4 min): Rt: 1.16 min, m/z: 326.3 [M+H]⁺.

### Example 16. Synthesis of Compound 16

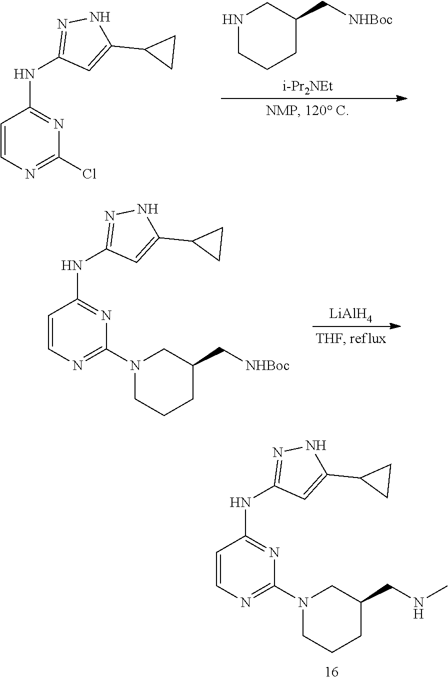 [SMILES: "i-Pr2NEt" ; Clc1nccc(Nc2cc(C3CC3)[nH]n2)n1 ; CC(C)(C)OC(=O)NC[C@H]1CCCNC1 ; CC(C)(C)OC(=O)NC[C@H]1CCCN(c2nccc(Nc3cc(C4CC4)[nH]n3)n2)C1 ; "LiAlH4" ; CNC[C@H]1CCCN(c2nccc(Nc3cc(C4CC4)[nH]n3)n2)C1]

### tert-Butyl N-[[(3R)-1-[4-[(5-Cyclopropyl-1H-pyrazol-3-yl)amino]pyrimidin-2-yl]-3-piperidyl]methyl]carbamate

A solution of 2-chloro-N-(5-cyclopropyl-1H-pyrazol-3-yl)pyrimidin-4-amine (367 mg, 1.56 mmol), tert-butyl N-[[(3S)-3-piperidyl]methyl]carbamate (501 mg, 2.34 mmol) and N,N-diisopropylethylamine (0.68 mL, 3.9 mmol) in NMP (6 mL) was heated to 120° C. and stirred for 16 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude material was purified by automated flash column chromatography, eluting with a gradient of CH₂Cl₂ to 1:9 2 M NH₃ in MeOH:CH₂Cl₂, to afford the title compound as a solid (471 mg, 1.12 mmol, 72%)

¹H NMR (400 MHz, DMSO-d₆) δ 11.91 (s, 1H), 9.28 (s, 1H), 7.84 (d, J=5.6 Hz, 1H), 6.89-6.72 (m, 1H), 6.33-5.97 (m, 2H), 4.62-4.23 (m, 2H), 2.86 (t, J=6.6 Hz, 3H), 2.70-2.52 (m, 1H), 1.93-1.81 (m, 1H), 1.80-1.73 (m, 1H), 1.64 (s, 1H), 1.40-1.35 (m, 10H), 1.27-1.13 (m, 1H), 1.01-0.85 (m, 2H), 0.74-0.56 (m, 2H).

UPLC-MS (basic method, 2 min): Rt: 1.07 min, m/z: 414.5 [M+H]⁺

### N-(5-Cyclopropyl-1H-pyrazol-3-yl)-2-[(3R)-3-(methylaminomethyl)-1-piperidyl]pyrimidin-4-amine

To a solution of tert-butyl N-[[(3R)-1-[4-[(5-Cyclopropyl-1H-pyrazol-3-yl)amino]pyrimidin-2-yl]-3-piperidyl]methyl]carbamate (180 mg, 0.435 mmol) in anhydrous THE (10 mL) was added a 2.4 M solution of lithium aluminum hydride in THE (1.8 mL, 4.4 mmol) dropwise at 0° C. The reaction mixture was heated to reflux and stirred for 2 hours. The reaction mixture was then cooled to 0° C., then quenched with water (180 μL), then a 15% w/v sodium hydroxide solution (180 μL), followed by water (540 μL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (13 mg, 39 μmol, 8%).

¹H NMR (400 MHz, DMSO-d₆) δ 11.91 (s, 1H), 9.32 (s, 1H), 7.83 (d, J=5.7 Hz, 1H), 6.15 (s, 2H), 4.56 (d, J=12.7 Hz, 1H), 4.44 (d, J=13.3 Hz, 1H), 2.91-2.83 (m, 1H), 2.68-2.56 (m, 1H), 2.36 (d, J=6.7 Hz, 2H), 2.26 (s, 3H), 1.89-1.75 (m, 2H), 1.69-1.51 (m, 2H), 1.37 (q, J=12.0 Hz, 1H), 1.32-1.09 (m, 1H), 1.00-0.86 (m, 2H), 0.74-0.61 (m, 2H).

UPLC-MS (basic method, 4 min): Rt: 1.26 min, m/z: 328.3 [M+H]⁺.

### Example 17. Synthesis of Compound 17

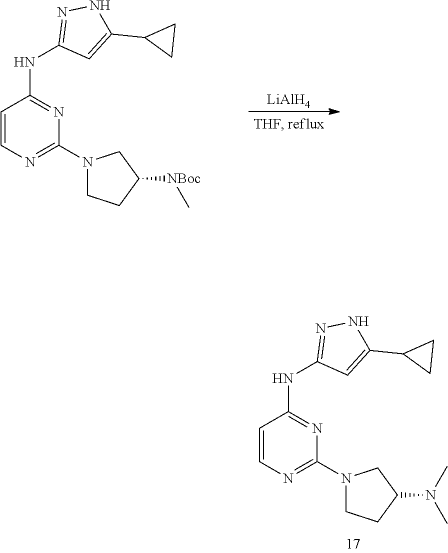

### N-(5-cyclopropyl-1H-pyrazol-3-yl)-2-[(3R)-3-(dimethylamino)pyrrolidin-1-yl]pyrimidin-4-amine

To a solution of tert-butyl N-[(3R)-1-[4-[(5-cyclopropyl-1H-pyrazol-3-yl)amino]pyrimidin-2-yl]pyrrolidin-3-yl]-N-methyl-carbamate (94 mg, 0.235 mmol) in anhydrous THF (10 mL) was added a 2.4 M solution of lithium aluminum hydride in THE (1.0 mL, 2.4 mmol) dropwise at 0° C. The reaction mixture was heated to reflux and stirred for 3 hours. The reaction mixture was then cooled to 0° C., then quenched with water (180 μL), then a 15% w/v sodium hydroxide solution (180 μL), followed by water (540 μL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (50 mg, 0.16 mmol, 67%).

¹H NMR (400 MHz, DMSO-d₆) δ 11.91 (s, 1H), 9.34 (s, 1H), 7.83 (d, J=5.7 Hz, 1H), 6.22 (s, 2H), 3.76 (dd, J=10.6, 7.1 Hz, 1H), 3.66 (t, J=9.7 Hz, 1H), 3.36 (s, 1H), 3.13 (t, J=9.5 Hz, 1H), 2.71 (dt, J=15.9, 8.1 Hz, 1H), 2.20 (s, 6H), 2.14-2.10 (m, 1H), 1.91-1.82 (m, 1H), 1.78-1.71 (m, 1H), 0.94-0.86 (m, 2H), 0.66-0.56 (m, 2H).

UPLC-MS (basic method, 4 min): Rt: 1.23 min, m/z: 314.3 [M+H]⁺.

### Example 18. Synthesis of Compound 18

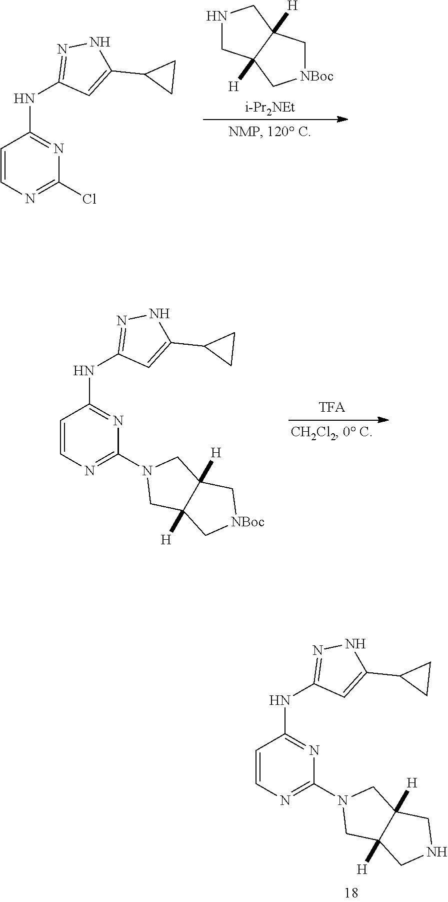 [SMILES: "i-Pr2NEt" ; Clc1nccc(Nc2cc(C3CC3)[nH]n2)n1 ; CC(C)(C)OC(=O)N1C[C@H]2CNC[C@H]2C1 ; CC(C)(C)OC(=O)N1C[C@@H]2CN(c3nccc(Nc4cc(C5CC5)[nH]n4)n3)C[C@@H]2C1 ; c1cc(Nc2cc(C3CC3)[nH]n2)nc(N2C[C@H]3CNC[C@H]3C2)n1]

### tert-Butyl rac-(3aS,6aR)-2-[4-[(5-Cyclopropyl-1H-pyrazol-3-yl)amino]pyrimidin-2-yl]-1,3,3a,4,6,6a hexahydropyrrolo[3,4-c]pyrrole-5-carboxylate

A solution of 2-chloro-N-(5-cyclopropyl-1H-pyrazol-3-yl)pyrimidin-4-amine (500 mg, 2.35 mmol), cis-2-Boc-hexahydropyrrol[3,4-c]pyrrole (370 mg, 1.57 mmol) and N,N-diisopropylethylamine (0.68 mL, 3.9 mmol) in NMP (5 mL) was heated to 120° C. and stirred for 20 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as an oil (607 mg, 1.28 mmol, 82%)

¹H NMR (400 MHz, DMSO-d₆) δ 11.89 (s, 1H), 9.35 (s, 1H), 7.83 (d, J=5.7 Hz, 1H), 6.21 (s, 2H), 3.68 (s, 2H), 3.31 (d, J=7.0 Hz, 1H), 3.14 (s, 2H), 2.93 (s, 2H), 2.69 (s, 1H), 2.17 (t, J=8.1 Hz, 1H), 1.95-1.82 (m, 2H), 1.38 (s, 9H), 0.97-0.86 (m, 2H), 0.68-0.62 (m, 2H).

UPLC-MS (basic method, 2 min): Rt: 1.02 min, m/z: 412.4 [M+H]⁺.

### N-(5-Cyclopropyl-1H-pyrazol-3-yl)-2-[rac-(3aR,6aS)-2,3,3a,4,6,6a-hexahydro-1H-pyrolo[3,4-c]pyrrol-5-yl]pyrimidin-4-amine

To a solution of tert-butyl rac-(3aS,6aR)-2-[4-[(5-cyclopropyl-1H-pyrazol-3-yl)amino]pyrimidin-2-yl]-1,3,3a,4,6,6a hexahydropyrrolo[3,4-c]pyrrole-5-carboxylate (200 mg, 0.48 mmol) in CH₂Cl₂ (4 mL) was added trifluoracetic acid (4.0 mL, 52 mmol) at 0° C. The reaction mixture was held at 0° C. and stirred for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC, to afford the title compound as a solid (38 mg, 0.115 mmol, 24%).

¹H NMR (400 MHz, DMSO-d₆) δ 11.89 (s, 1H), 9.36 (s, 1H), 7.83 (d, J=5.7 Hz, 1H), 6.20 (s, 2H), 3.68 (dd, J=11.1, 7.3 Hz, 2H), 3.52 (s, 1H), 3.15 (d, J=11.4 Hz, 1H), 2.95-2.88 (m, 2H), 2.77 (s, 2H), 2.64-2.57 (m, 2H), 1.86 (td, J=8.6, 4.4 Hz, 1H), 0.95-0.86 (m, 2H), 0.69-0.61 (m, 2H).

¹H NMR (400 MHz, methanol-d₄) δ 7.85 (d, J=5.9 Hz, 1H), 6.16 (s, 2H), 3.75 (dd, J=11.4, 7.1 Hz, 2H), 3.53 (d, J=3.2 Hz, 1H), 3.50 (d, J=3.2 Hz, 1H), 3.22 (dd, J=11.5, 7.1 Hz, 2H), 3.01 (d, J=3.8 Hz, 2H), 2.86 (d, J=3.9 Hz, 1H), 2.83 (d, J=4.0 Hz, 1H), 2.07-1.72 (m, 1H), 0.97 (d, J=8.3 Hz, 2H), 0.88-0.53 (m, 2H).

UPLC-MS (basic method, 4 min): Rt: 0.99 min, m/z: 312.3 [M+H]⁺.

### Example 19. Synthesis of Compound 19

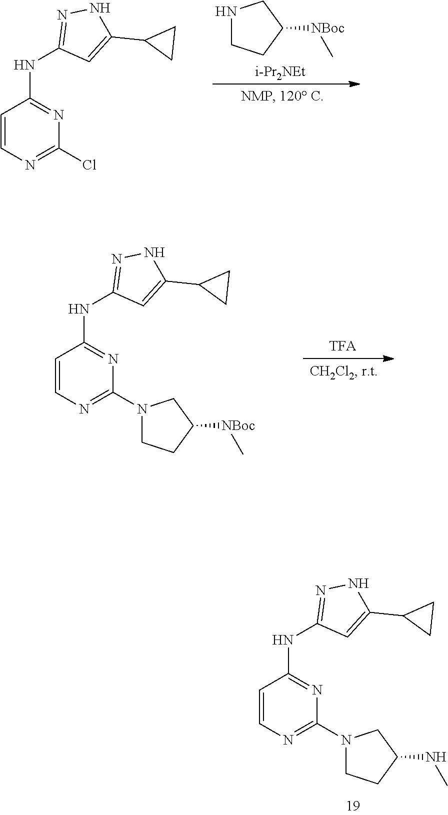 [SMILES: "i-Pr2NEt" ; Clc1nccc(Nc2cc(C3CC3)[nH]n2)n1 ; CN(C(=O)OC(C)(C)C)[C@@H]1CCNC1 ; CN(C(=O)OC(C)(C)C)[C@@H]1CCN(c2nccc(Nc3cc(C4CC4)[nH]n3)n2)C1 ; CN[C@@H]1CCN(c2nccc(Nc3cc(C4CC4)[nH]n3)n2)C1 ; "TFA"]

### tert-Butyl N-[(3R)-1-[4-[(5-Cyclopropyl-1H-pyrazol-3-yl)amino]pyrimidin-2-yl]pyrrolidin-3-yl]-N-methyl-carbamate

A solution of 2-chloro-N-(5-cyclopropyl-1H-pyrazol-3-yl)pyrimidin-4-amine (200 mg, 0.849 mmol), (R)-tert-butyl methyl(pyrrolidin-3-yl)carbamate (255 mg, 1.27 mmol) and N,N-diisopropylethylamine (0.37 mL, 2.1 mmol) in NMP (5 mL) was heated to 120° C. and stirred for 18 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude material was purified by automated flash column chromatography, over silica gel, eluting with a gradient of 1:4 EtOAc:iso-hexane to 4:6 EtOAc:iso-hexane to afford the title compound as an oil (219 mg, 0.450 mmol, 82%)

¹H NMR (400 MHz, DMSO-d₆) δ 11.90 (s, 1H), 9.35 (s, 1H), 7.84 (d, J=5.7 Hz, 1H), 6.24 (s, 2H), 4.64 (s, 1H), 3.66 (s, 2H), 3.41 (d, J=9.8 Hz, 2H), 2.75 (s, 3H), 2.06 (d, J=8.2 Hz, 2H), 1.76 (s, 1H), 1.42 (s, 9H), 0.91 (m, 2H), 0.69-0.56 (m, 2H).

UPLC-MS (basic method, 2 min): Rt: 0.93 min, m/z: 400.4 [M+H]⁺.

### N-(5-Cyclopropyl-1H-pyrazol-3-yl)-2-[(3R)-3-(methylamino)pyrrolidin-1-yl]pyrimidin-4-amine

To a solution of tert-butyl N-[(3R)-1-[4-[(5-cyclopropyl-1H-pyrazol-3-yl)amino]pyrimidin-2-yl]pyrrolidin-3-yl]-N-methyl-carbamate (85 mg, 0.213 mmol) in CH₂Cl₂ (3 mL) was added trifluoracetic acid (3.0 mL, 39 mmol) and the reaction mixture was stirred at ambient temperature for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase acidic prepHPLC, using TFA as the additive, followed by reversed-phase basic prepHPLC, using ammonia as the additive, to afford the title compound as a solid (22 mg, 73 μmol, 35%).

¹H NMR (400 MHz, DMSO-d₆, 90° C.) δ 8.83 (s, 1H), 7.85 (d, J=5.6 Hz, 1H), 6.21 (d, J=5.7 Hz, 1H), 6.11 (s, 1H), 3.70-3.61 (m, 1H), 3.61-3.55 (m, 1H), 3.52-3.44 (m, 1H), 3.33-3.21 (m, 2H), 2.35 (s, 3H), 2.12-1.99 (m, 1H), 1.90-1.82 (m, 1H), 1.78-1.70 (m, 1H), 0.96-0.83 (m, 2H), 0.70-0.61 (m, 2H).

UPLC-MS (basic method, 4 min): Rt: 1.02 min, m/z: 300.1 [M+H]⁺.

### Example 20. Synthesis of Compound 20

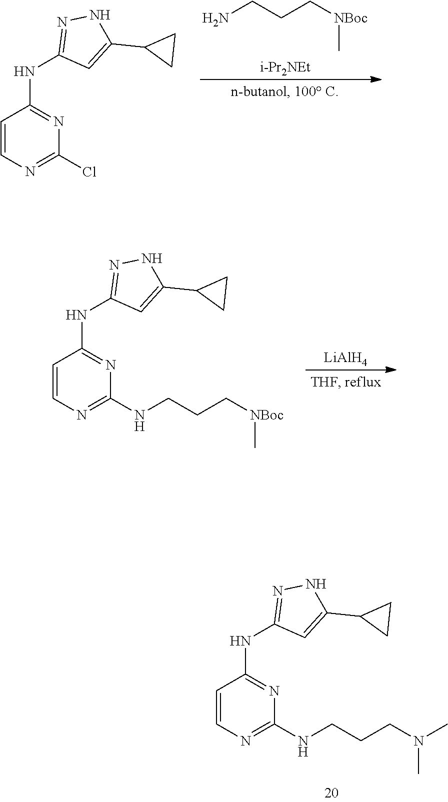 [SMILES: "i-Pr2NEt" ; Clc1nccc(Nc2cc(C3CC3)[nH]n2)n1 ; CN(CCCN)C(=O)OC(C)(C)C ; CN(CCCNc1nccc(Nc2cc(C3CC3)[nH]n2)n1)C(=O)OC(C)(C)C ; "LiAlH4" ; CN(C)CCCNc1nccc(Nc2cc(C3CC3)[nH]n2)n1]

### tert-Butyl N-[3-[[4-[(5-Cyclopropyl-1H-pyrazol-3-yl)amino]pyrimidin-2-yl]amino]propyl]-N-methyl-carbamate

A solution of 2-chloro-N-(5-cyclopropyl-1H-pyrazol-3-yl)pyrimidin-4-amine (300 mg, 1.27 mmol), 3-(N-Boc-N-methylamino)propylamine (610 μL, 3.18 mmol) and N,N-diisopropylethylamine (0.78 mL, 4.5 mmol) in n-butanol (7 mL) was heated to 100° C. and stirred for 44 hours. The reaction mixture was then cooled to ambient temperature, then concentrated to dryness under reduced pressure. The residue was suspended in water (20 mL) and extracted with CH₂Cl₂ (3×20 mL). The combined organic extracts were dried over Na₂SO₄, then concentrated to dryness under reduced pressure. The crude material was purified by automated flash column chromatography, over silica gel, eluting with a gradient of CH₂Cl₂ to 9:1 2 M NH₃ in MeOH:CH₂Cl₂ to afford the title compound as a solid (270 mg, 0.665 mmol, 51%)

¹H NMR (400 MHz, chloroform-d) δ 7.97 (dd, J=13.1, 5.8 Hz, 1H), 6.24 (s, 1H), 5.87 (s, 1H), 3.51-3.39 (m, 3H), 3.10 (s, 1H), 2.87 (s, 3H), 1.88 (td, J=8.7, 4.3 Hz, 2H), 1.53-1.41 (m, 10H), 0.95 (t, J=6.8 Hz, 2H), 0.77 (d, J=5.6 Hz, 2H).

UPLC-MS (basic method, 2 min): Rt: 1.01 min, m/z: 388.4 [M+H]⁺.

### N₄-(5-Cyclopropyl-1H-pyrazol-3-yl)-N₂-[3-(dimethylamino)propyl]pyrimidine-2,4-diamine

To a solution of tert-butyl N-[3-[[4-[(5-cyclopropyl-1H-pyrazol-3-yl)amino]pyrimidin-2-yl]amino]propyl]-N-methyl-carbamate (159 mg, 0.410 mmol) in anhydrous THF (10 mL) was added a 2.4 M solution of lithium aluminum hydride in THF (1.7 mL, 4.1 mmol) dropwise at 0° C. The reaction mixture was heated to reflux and stirred for 3 hours. The reaction mixture was then cooled to 0° C., then quenched with water (1.5 mL), then a 15% w/v sodium hydroxide solution (1.5 mL), followed by water (4.5 mL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (25 mg, 80 μmol, 19%).

¹H NMR (400 MHz, DMSO-d₆) δ 11.87 (s, 1H), 9.22 (s, 1H), 7.75 (s, 1H), 6.57 (s, 1H), 6.33-5.96 (m, 2H), 3.24 (q, J=6.7 Hz, 2H), 2.24 (t, J=7.1 Hz, 2H), 2.11 (s, 6H), 1.83 (s, 1H), 1.69-1.60 (m, 2H), 0.90 (s, 2H), 0.65 (s, 2H).

UPLC-MS (basic method, 4 min): Rt: 1.11 min, m/z: 302.1 [M+H]⁺.

### Example 21. Synthesis of Compound 21

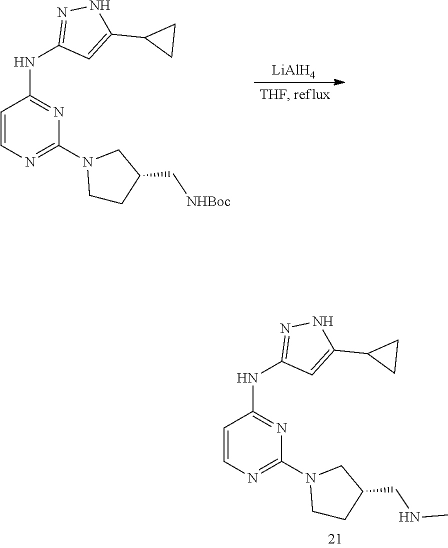 [SMILES: CC(C)(C)OC(=O)NC[C@@H]1CCN(c2nccc(Nc3cc(C4CC4)[nH]n3)n2)C1 ; "LiAlH4" ; CNC[C@@H]1CCN(c2nccc(Nc3cc(C4CC4)[nH]n3)n2)C1]

### N-(5-Cyclopropyl-1H-pyrazol-3-yl)-2-[(3S)-3-(methylaminomethyl)pyrrolidin-1-yl]pyrimidin-4-amine

To a solution of tert-butyl N-[[(3S)-1-[4-[(5-cyclopropyl-1H-pyrazol-3-yl)amino]pyrimidin-2-yl]pyrrolidin-3-yl]methyl]carbamate (495 mg, 1.24 mmol) in anhydrous THF (15 mL) was added a 2.4 M solution of lithium aluminum hydride in THF (5.2 mL, 12 mmol) dropwise at 0° C. The reaction mixture was heated to reflux and stirred for 2 hours. The reaction mixture was then cooled to 0° C., then quenched with water (510 μL), then a 15% w/v sodium hydroxide solution (510 μL), followed by water (1.5 mL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (20 mL). The eluent was then concentrated to dryness under reduced pressure. The residue was purified by reversed-phase acidic prepHPLC, using TFA as a modified, followed by reversed-phase basic prepHPLC, using ammonia as a modified, to afford the title compound as a solid (91 mg, 0.29 mmol, 23%).

¹H NMR (400 MHz, DMSO-d₆) δ 11.80 (s, 1H), 9.33 (s, 1H), 7.82 (d, J=5.7 Hz, 1H), 6.20 (s, 2H), 3.69-3.51 (m, 2H), 3.43-3.39 (m, 1H), 3.20-3.11 (m, 1H), 2.62-2.50 (m, 2H), 2.45-2.34 (m, 1H), 2.33 (s, 3H), 2.09-1.97 (m, 1H), 1.85 (tt, J=8.4, 5.1 Hz, 1H), 1.65 (dd, J=12.2, 8.0 Hz, 1H), 0.95-0.84 (m, 2H), 0.69-0.61 (m, 2H).

UPLC-MS (basic method, 4 min): Rt: 1.04 min, m/z: 314.3 [M+H]⁺.

### Example 22. Synthesis of Compound

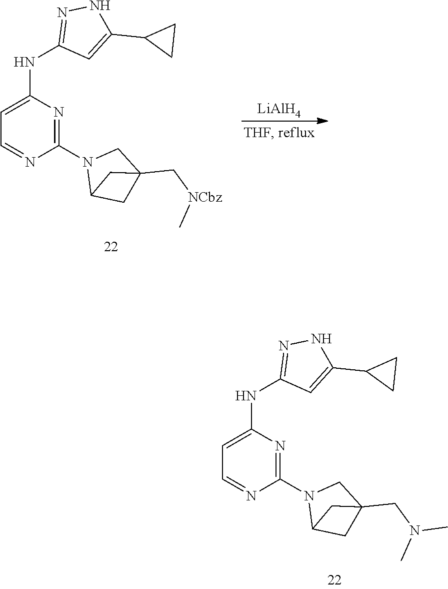 [SMILES: CN(CC12CC(C1)N(c1nccc(Nc3cc(C4CC4)[nH]n3)n1)C2)C(=O)OCc1ccccc1 ; "LiAlH4" ; CN(C)CC12CC(C1)N(c1nccc(Nc3cc(C4CC4)[nH]n3)n1)C2]

### N-(5-Cyclopropyl-1H-pyrazol-3-yl)-2-[4-[(dimethylamino)methyl]-2-azabicyclo[2.1.1]hexan-2-yl]pyrimidin-4-amine

To a solution of benzyl ((2-(4-((5-cyclopropyl-1H-pyrazol-3-yl)amino)pyrimidin-2-yl)-2-azabicyclo[2.1.1]hexan-4-yl)methyl)(methyl)carbamate (149 mg, 0.324 mmol) in anhydrous THF (3 mL) was added a 2.4 M solution of lithium aluminum hydride in THF (1.4 mL, 3.3 mmol) dropwise at 0° C. The reaction mixture was heated to reflux and stirred for 1 hour. The reaction mixture was then cooled to 0° C., then quenched with water (140 μL), then a 15% w/v sodium hydroxide solution (140 μL), followed by water (450 μL). The reaction mixture was warmed to ambient temperature and stirred for 15 minutes, after which magnesium sulfate (ca. 1 g) was added, and the reaction mixture was stirred for a further 15 minutes. The reaction mixture was passed through a pad of celite, and the filter cake was washed with EtOAc (20 mL). The eluent was then concentrated to dryness under reduced pressure. The crude residue was loaded onto an SCX cartridge, the cartridge was then washed with methanol, then the compound was eluted with 2 M NH₃ in methanol. The eluent was concentrated under reduced pressure to afford the title compound as a solid (73 mg, 0.21 mmol, 65%).

¹H NMR (400 MHz, DMSO-d₆) δ 11.90 (s, 1H), 9.30 (s, 1H), 7.83 (d, J=5.6 Hz, 1H), 6.41-6.03 (m, 2H), 4.68 (s, 1H), 3.35 (s, 2H), 2.55 (s, 2H), 2.16 (s, 6H), 1.86 (td, J=8.3, 4.2 Hz, 1H), 1.79 (d, J=4.2 Hz, 2H), 1.44 (dd, J=4.3, 1.8 Hz, 2H), 0.91 (d, J=6.1 Hz, 2H), 0.70-0.61 (m, 2H).

UPLC-MS (basic method, 4 min): Rt: 1.37 min, m/z: 340.1 [M+H]⁺.

### Example 23. Synthesis of Compound 23

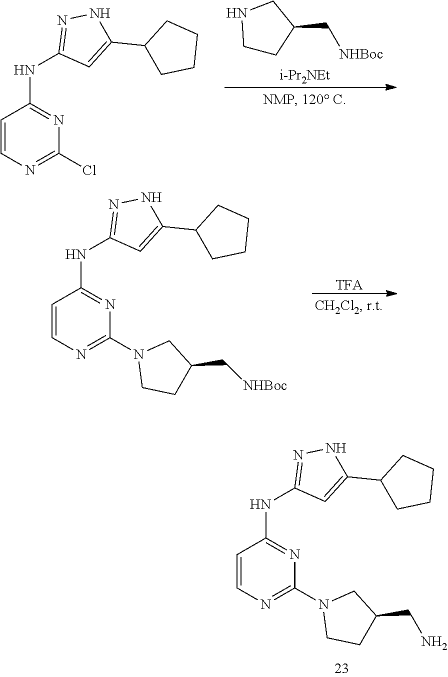

### tert-butyl N-[[(3R)-1-[4-[(5-cyclopentyl-1H-pyrazol-3-yl)amino]pyrimidin-2-yl]pyrrolidin-3-yl]methyl]carbamate

A solution of 2-chloro-N-(5-cyclopropyl-1H-pyrazol-3-yl)pyrimidin-4-amine (298 mg, 1.13 mmol), (S)-tert-Butyl (pyrrolidin-3-ylmethyl)carbamate (357 mg, 1.78 mmol) and N,N-diisopropylethylamine (0.49 mL, 2.82 mmol) in NMP (6 mL) was heated to 120° C. and stirred for 48 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as a foam (576 mg, 1.01 mmol, 89%).

¹H NMR (400 MHz, chloroform-d) δ 8.01 (d, J=5.7 Hz, 1H), 6.91 (s, 1H), 6.09 (s, 2H), 4.71 (s, 1H), 3.83-3.67 (m, 2H), 3.56 (q, J=8.1 Hz, 1H), 3.27 (t, J=9.0 Hz, 2H), 3.21-2.99 (m, 3H), 2.58-2.46 (m, 1H), 2.17-2.07 (m, 2H), 1.84-1.64 (m, 6H), 1.45 (s, 9H).

UPLC-MS (basic method, 2 min) rt=1.13 min, m/z=428.2 [M+H]⁺.

### 2-[(3R)-3-(aminomethyl)pyrrolidin-1-yl]-N-(5-cyclopentyl-1H-pyrazol-3-yl)pyrimidin-4-amine

To a solution of tert-butyl N-[[(3R)-1-[4-[(5-cyclopentyl-1H-pyrazol-3-yl)amino]pyrimidin-2-yl]pyrrolidin-3-yl]methyl]carbamate (102 mg, 0.178 mmol) in CH₂Cl₂ (2 mL) was added trifluoroacetic acid (2.0 mL, 26 mmol) and the reaction mixture was stirred at ambient temperature for 15 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC, to afford the title compound as a solid (30 mg, 92 μmol, 52%).

¹H NMR (400 MHz, methanol-d₄) δ 7.76 (d, J=7.0 Hz, 1H), 6.42 (s, 2H), 3.25-2.98 (m, 4H), 2.83-2.66 (m, 1H), 2.48-2.29 (m, 1H), 2.18-1.87 (m, 3H), 1.89-1.58 (m, 6H), 1.31 (d, J=15.4 Hz, 3H).

UPLC-MS (basic method, 4 min): rt=1.27 min, m/z=328.1 [M+H]⁺.

### Example 24. Synthesis of Compound 24

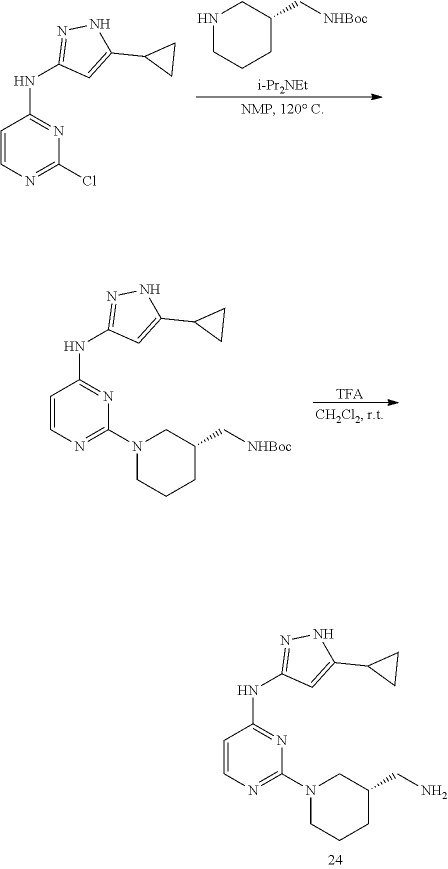

### tert-Butyl N-[[(3S)-1-[4-[(5-cyclopropyl-1H-pyrazol-3-yl)amino]pyrimidin-2-yl]-3-piperidyl]methyl]carbamate

A solution of 2-chloro-N-(5-cyclopropyl-1H-pyrazol-3-yl)pyrimidin-4-amine (300 mg, 1.27 mmol), (R)-tert-butyl (piperidin-3-ylmethyl)carbamate (409 mg, 1.91 mmol) and N,N-diisopropylethylamine (0.55 mL, 3.18 mmol) in NMP (6 mL) was heated to 120° C. and stirred for 18 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as an oil (414 mg, 0.80 mmol, 63%).

¹H NMR (400 MHz, DMSO-d₆) δ 11.61 (s, 1H), 8.86 (s, 1H), 7.86 (d, J=5.7 Hz, 1H), 6.24 (d, J=5.8 Hz, 1H), 6.09 (s, 1H), 3.64-3.53 (m, 2H), 3.50-3.40 (m, 3H), 3.30-3.20 (m, 2H), 2.01-1.80 (m, 6H), 1.43 (s, 9H), 0.94-0.86 (m, 2H), 0.71-0.60 (m, 2H).

UPLC-MS (basic method, 2 min) r.t.: 1.07 min; m/z: 414.4 [M+H]⁺.

### 2-[(3S)-3-(aminomethyl)-1-piperidyl]-N-(5-cyclopropyl-1H-pyrazol-3-yl)pyrimidin-4-amine

To a solution of tert-butyl N-[[(3S)-1-[4-[(5-cyclopropyl-1H-pyrazol-3-yl)amino]pyrimidin-2-yl]-3-piperidyl]methyl]carbamate (100 mg, 0.242 mmol) in CH₂Cl₂ (2 mL) was added trifluoroacetic acid (2.0 mL, 26 mmol) and the reaction mixture was stirred at ambient temperature for 15 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC, using ammonia as the additive, to afford the title compound as a solid (49 mg, 0.155 mmol, 64%).

¹H NMR (400 MHz, DMSO-d₆) δ 11.80 (s, 1H), 9.32 (s, 1H), 7.84 (d, J=5.6 Hz, 1H), 6.36-5.94 (m, 2H), 4.57-4.32 (m, 2H), 2.96-2.80 (m, 1H), 2.66 (dd, J=13.1, 9.5 Hz, 1H), 2.54 (s, 1H), 2.45 (d, J=6.6 Hz, 2H), 1.96-1.73 (m, 2H), 1.63 (dt, J=7.5, 3.4 Hz, 1H), 1.50-1.28 (m, 2H), 1.27-1.09 (m, 1H), 1.00-0.81 (m, 2H), 0.74-0.56 (m, 2H).

UPLC-MS (basic method, 4 min): rt=1.10 min, m/z=314.1 [M+H]⁺.

### Example 25. Synthesis of Compound 25

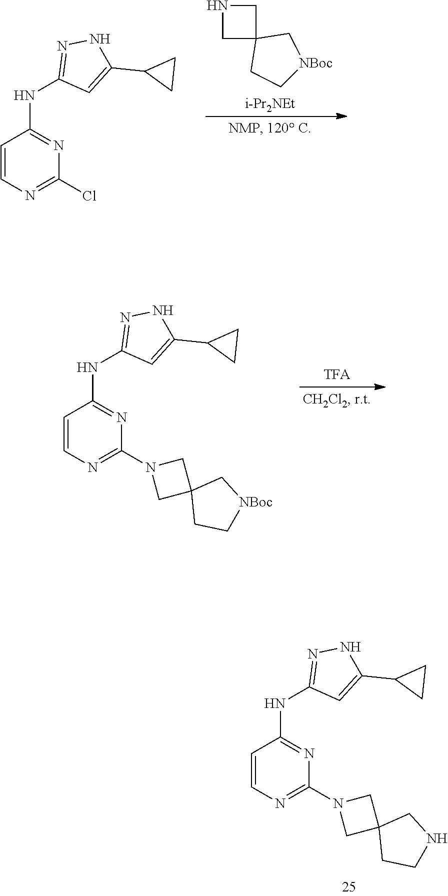 [SMILES: "i-Pr2NEt" ; Clc1nccc(Nc2cc(C3CC3)[nH]n2)n1 ; CC(C)(C)OC(=O)N1CCC2(CNC2)C1 ; CC(C)(C)OC(=O)N1CCC2(C1)CN(c1nccc(Nc3cc(C4CC4)[nH]n3)n1)C2 ; c1cc(Nc2cc(C3CC3)[nH]n2)nc(N2CC3(CCNC3)C2)n1]

### tert-butyl 2-[4-[(5-cyclopropyl-1H-pyrazol-3-yl)amino]pyrimidin-2-yl]-2,7-diazaspiro[3.4]octane-7-carboxylate

A solution of 2-chloro-N-(5-cyclopropyl-1H-pyrazol-3-yl)pyrimidin-4-amine (305 mg, 1.29 mmol), tert-butyl 2,6-diazaspiro[3.4]octane-6-carboxylate (431 mg, 2.03 mmol) and N,N-diisopropylethylamine (0.56 mL, 3.22 mmol) in NMP (6 mL) was heated to 120° C. and stirred for 48 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (797 mg, 1.86 mmol, 143%)

¹H NMR (400 MHz, chloroform-d) δ 7.97 (d, J=5.9 Hz, 1H), 4.08-3.96 (m, 4H), 3.53 (d, J=11.4 Hz, 2H), 2.11 (m, 2H), 1.86 (m, 1H), 1.46 (d, J=5.7 Hz, 9H), 1.01 (d, J=6.6 Hz, 2H), 0.95 (d, J=7.2 Hz, 2H), 0.77-0.68 (m, 2H). Compound contaminated with amine starting material.

UPLC-MS (basic method, 2 min) rt=1.06 min, m/z=412.2 [M+H]⁺.

### N-(5-cyclopropyl-1H-pyrazol-3-yl)-2-(2,7-diazaspiro[3.4]octan-2-yl)pyrimidin-4-amine

To a solution of tert-butyl 2-[4-[(5-cyclopropyl-1H-pyrazol-3-yl)amino]pyrimidin-2-yl]-2,7-diazaspiro[3.4]octane-7-carboxylate (105 mg, 0.246 mmol) in CH₂Cl₂ (2 mL) was added trifluoroacetic acid (2.0 mL, 26 mmol) and the reaction mixture was stirred at ambient temperature for 15 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC, to afford the title compound as a solid (7.6 mg, 0.024 mmol, 9.7%).

¹H NMR (400 MHz, DMSO-d₆) δ 11.91 (s, 1H), 9.46 (s, 1H), 7.85 (d, J=5.7 Hz, 1H), 6.32 (s, 1H), 6.16 (s, 1H), 3.98 (d, J=8.7 Hz, 2H), 3.91 (d, J=8.5 Hz, 2H), 3.28 (s, 2H), 3.13 (t, J=7.3 Hz, 2H), 2.14 (t, J=7.3 Hz, 2H), 1.84 (tt, J=8.4, 5.1 Hz, 1H), 0.95-0.81 (m, 2H), 0.70-0.62 (m, 2H).

UPLC-MS (basic method, 4 min): rt=0.97 min, m/z=312.1 [M+H]⁺.

### Example 26. Synthesis of Compound 26

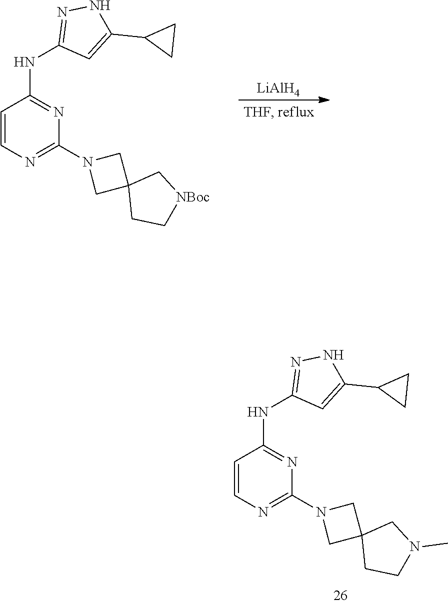 [SMILES: CC(C)(C)OC(=O)N1CCC2(C1)CN(c1nccc(Nc3cc(C4CC4)[nH]n3)n1)C2 ; "LiAlH4" ; CN1CCC2(C1)CN(c1nccc(Nc3cc(C4CC4)[nH]n3)n1)C2]

### N-(5-cyclopropyl-1H-pyrazol-3-yl)-2-(7-methyl-2,7-diazaspiro[3.4]octan-2-yl)pyrimidin-4-amine

To a solution of tert-butyl 2-[4-[(5-cyclopropyl-1H-pyrazol-3-yl)amino]pyrimidin-2-yl]-2,7-diazaspiro[3.4]octane-7-carboxylate (160 mg, 0.37 mmol) in anhydrous THF (3.5 mL) was added a 2.4 M solution of lithium aluminum hydride in THF (1.55 mL, 3.672 mmol) dropwise at 0° C. The reaction mixture was heated to reflux and stirred for 3 hours. The reaction mixture was then cooled to 0° C., then quenched with water (0.15 mL), then a 10% w/v sodium hydroxide solution (0.15 mL), followed by water (0.5 mL). The reaction mixture was warmed to ambient temperature and stirred for 15 minutes, after which magnesium sulfate (ca. 1 g) was added and the reaction mixture was stirred for a further 15 minutes. The reaction mixture was filtered, and the filter cake was washed with EtOAc (20 mL). The eluent was then concentrated to dryness under reduced pressure. The residue was purified by reversed-phase basic prepHPLC, to afford the title compound as a solid (30 mg, 0.092 mmol, 25%).

¹H NMR (400 MHz, DMSO-d₆) δ 11.89 (s, 1H), 9.40 (s, 1H), 7.83 (d, J=5.7 Hz, 1H), 6.34 (s, 1H), 6.17 (s, 1H), 3.88 (q, J=8.4 Hz, 4H), 2.64 (s, 2H), 2.46 (t, J=7.0 Hz, 2H), 2.23 (s, 3H), 2.04 (t, J=7.0 Hz, 2H), 1.85 (td, J=8.4, 4.3 Hz, 1H), 0.91 (d, J=6.2 Hz, 2H), 0.69-0.61 (m, 2H).

UPLC-MS (basic method, 4 min): rt=1.20 min, m/z=326.1 [M+H]+.

### Example 27. Synthesis of Compound 27

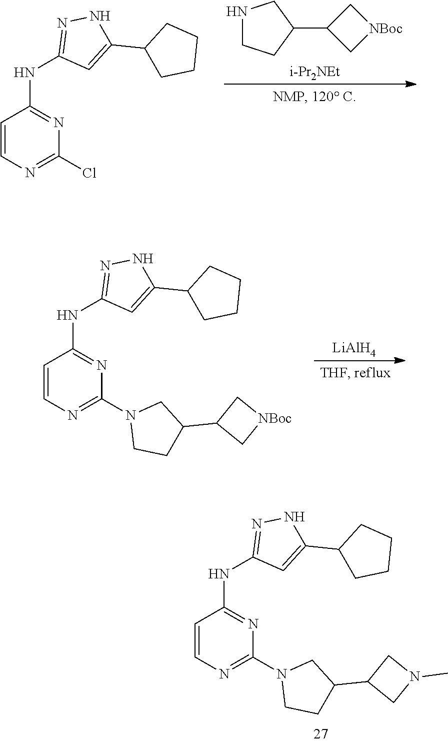

### tert-Butyl 3-[1-[4-[(5-Cyclopentyl-1H-pyrazol-3-yl)amino]pyrimidin-2-yl]pyrrolidin-3-yl]azetidine-1-carboxylate

A solution of 2-chloro-N-(5-cyclopropyl-1H-pyrazol-3-yl)pyrimidin-4-amine (469 mg, 1.78 mmol), tert-butyl 3-(pyrrolidin-3-yl)azetidine-1-carboxylate (550 mg, 2.43 mmol) and N,N-diisopropylethylamine (0.77 mL, 4.4 mmol) in NMP (9 mL) was heated to 120° C. and stirred for 48 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The rude residue was purified by automated flash column chromatography, over silica gel, eluting with a gradient of CH₂Cl₂ to 1:9 2 M NH₃ in MeOH:CH₂Cl₂ to afford the title compound as a foam (463 mg, 0.938 mmol, 53%).

¹H NMR (400 MHz, chloroform-d) δ 7.98 (s, 1H), 6.09 (s, 2H), 4.03 (q, J=7.8 Hz, 2H), 3.82-3.62 (m, 3H), 3.56 (dt, J=10.9, 7.8 Hz, 1H), 3.17 (dd, J=10.5, 6.7 Hz, 1H), 3.05 (dt, J=14.9, 7.3 Hz, 1H), 2.53 (d, J=3.2 Hz, 2H), 2.14-1.95 (m, 4H), 1.80-1.58 (m, 7H), 1.44 (s, 9H).

UPLC-MS (basic method, 2 min) rt=1.18 min, m/z=454.3 [M+H]⁺.

### N-(5-cyclopentyl-1H-pyrazol-3-yl)-2-[3-(1-methylazetidin-3-yl)pyrrolidin-1-yl]pyrimidin-4-amine

To a solution of tert-butyl 3-[1-[4-[(5-cyclopentyl-1H-pyrazol-3-yl)amino]pyrimidin-2-yl]pyrrolidin-3-yl]azetidine-1-carboxylate (134 mg, 0.37 mmol) in anhydrous THF (3 mL) was added a 2.4 M solution of lithium aluminum hydride in THF (1.1 mL, 2.7 mmol) dropwise at 0° C. The reaction mixture was heated to reflux and stirred for 3 hours. The reaction mixture was then cooled to 0° C., then quenched with water (0.13 mL), then a 10% w/v sodium hydroxide solution (0.13 mL), followed by water (0.4 mL). The reaction mixture was warmed to ambient temperature and stirred for 15 minutes, after which magnesium sulfate (ca. 1 g) was added and the reaction mixture was stirred for a further 15 minutes. The reaction mixture was filtered, then the filter cake was washed with EtOAc (20 mL). The eluent was then concentrated to dryness under reduced pressure. The residue was purified by reversed-phase basic prepHIPLC, to afford the title compound as a solid (83 mg, 0.217 mmol, 80%).

¹H NMR (400 MHz, DMSO-d₆) δ 11.85 (s, 1H), 9.34 (s, 1H), 7.82 (d, J=5.5 Hz, 1H), 6.42 (s, 1H), 6.18 (s, 1H), 3.64 (s, 1H), 3.62-3.53 (m, 1H), 3.44-3.23 (m, 2H), 3.09-2.94 (m, 2H), 2.90-2.80 (m, 2H), 2.47-2.29 (m, 2H), 2.20 (s, 3H), 2.05-1.92 (m, 3H), 1.75-1.44 (m, 8H).

UPLC-MS (basic method, 4 min): rt=1.40 min, m/z=368.1 [M+H]⁺.

### Example 28. Synthesis of Compound 28

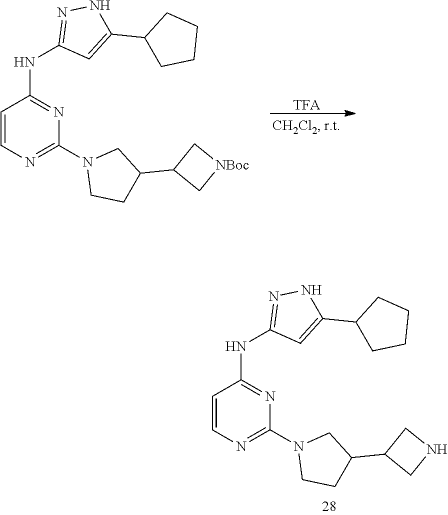 [SMILES: CC(C)(C)OC(=O)N1CC(C2CCN(c3nccc(Nc4cc(C5CCCC5)[nH]n4)n3)C2)C1 ; c1cc(Nc2cc(C3CCCC3)[nH]n2)nc(N2CCC(C3CNC3)C2)n1 ; "TFA"]

### 2-[3-(azetidin-3-yl)pyrrolidin-1-yl]-N-(5-cyclopentyl-1H-pyrazol-3-yl)pyrimidin-4-amine

To a solution of tert-butyl 3-[1-[4-[(5-cyclopentyl-1H-pyrazol-3-yl)amino]pyrimidin-2-yl]pyrrolidin-3-yl]azetidine-1-carboxylate (142 mg, 0.288 mmol) in CH₂Cl₂ (2 mL) was added trifluoroacetic acid (2.0 mL, 26 mmol) and the reaction mixture was stirred at ambient temperature for 15 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC, to afford the title compound as a solid (14 mg, 37 μmol, 13%).

¹H NMR (400 MHz, DMSO-d₆) δ 11.85 (s, 1H), 9.37 (s, 1H), 7.82 (d, J=5.7 Hz, 1H), 6.39 (s, 1H), 6.18 (s, 1H), 3.59-3.55 (m, 5H), 3.09-2.94 (m, 3H), 2.02-1.91 (m, 4H), 1.75-1.66 (m, 2H), 1.66-1.47 (m, 7H).

UPLC-MS (basic method, 4 min): rt=1.24 min, m/z=354.1 [M+H]⁺.

### Example 29. Synthesis of Compound 29

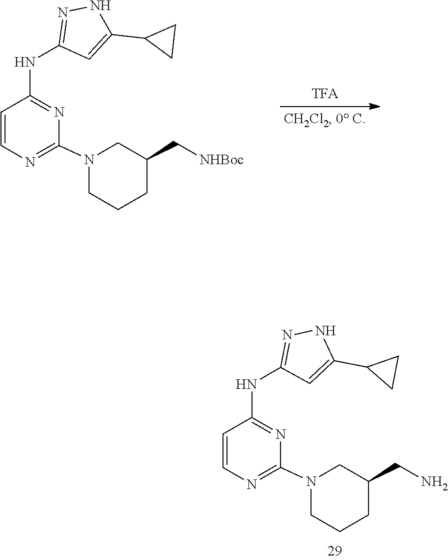 [SMILES: CC(C)(C)OC(=O)NC[C@H]1CCCN(c2nccc(Nc3cc(C4CC4)[nH]n3)n2)C1 ; NC[C@H]1CCCN(c2nccc(Nc3cc(C4CC4)[nH]n3)n2)C1]

### 2-[(3R)-3-(Aminomethyl)-1-piperidyl]-N-(5-cyclopropyl-1H-pyrazol-3-yl)pyrimidin-4-amine

To a solution of tert-butyl N-[[(3R)-1-[4-[(5-cyclopropyl-1H-pyrazol-3-yl)amino]pyrimidin-2-yl]-3-piperidyl]methyl]carbamate (171 mg, 0.41 mmol) in CH₂Cl₂ (4 mL) was added trifluoroacetic acid (4 mL, 52 mmol) at 0° C. The reaction mixture was warmed to ambient temperature and stirred for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC to afford the title compound as a solid (53 mg, 0.167 mmol, 40%).

¹H NMR (400 MHz, DMSO-d₆) δ 11.93 (s, 1H), 9.35 (s, 1H), 7.84 (d, J=4.6 Hz, 1H), 6.13 (s, 2H), 4.52 (d, J=12.3 Hz, 1H), 4.41 (d, J=13.5 Hz, 1H), 2.97-2.77 (m, 2H), 2.70-2.55 (m, 1H), 2.44 (d, J=6.6 Hz, 2H), 1.92-1.81 (m, 2H), 1.69-1.58 (m, 1H), 1.43-1.31 (m, 2H), 1.21-1.10 (m, 1H), 0.96-0.86 (m, 2H), 0.72-0.58 (m, 2H).

UPLC-MS (basic method, 4 min): Rt: 1.16 min, m/z: 314.1 [M+H]⁺.

### Example 30. Synthesis of Compound 31

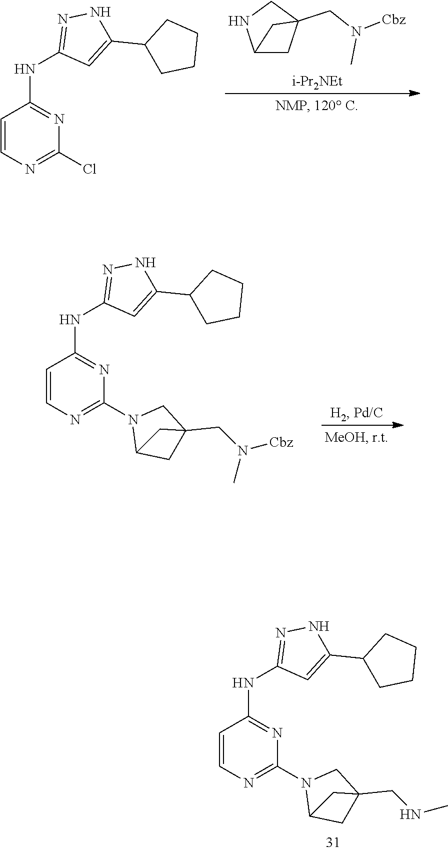 [SMILES: "i-Pr2NEt" ; Clc1nccc(Nc2cc(C3CCCC3)[nH]n2)n1 ; CN(CC12CNC(C1)C2)C(=O)OCc1ccccc1 ; CN(CC12CC(C1)N(c1nccc(Nc3cc(C4CCCC4)[nH]n3)n1)C2)C(=O)OCc1ccccc1 ; "H2, Pd/C" ; CNCC12CC(C1)N(c1nccc(Nc3cc(C4CCCC4)[nH]n3)n1)C2]

### Benzyl N-[[2-[4-[(5-cyclopentyl-1H-pyrazol-3-yl)amino]pyrimidin-2-yl]-2-azabicyclo[2.1.1]hexan-4-yl]methyl]-N-methyl-carbamate

A solution of 2-chloro-N-(5-cyclopentyl-1H-pyrazol-3-yl)pyrimidin-4-amine (185 mg, 0.702 mmol), benzyl N-(2-azabicyclo[2.1.1]hexan-4-ylmethyl)-N-methyl-carbamate (320 mg, 1.10 mmol) and N,N-diisopropylethylamine (0.74 mL, 1.84 mmol) in NMP (7 mL) was heated to 120° C. and stirred for 48 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (228 mg, 0.431 mmol, 61%)

¹H NMR (400 MHz, chloroform-d) δ 7.99 (d, J=5.7 Hz, 1H), 7.41-7.28 (m, 5H), 7.08 (s, 1H), 6.17 (s, 1H), 6.10-6.01 (m, 1H), 5.12 (s, 2H), 4.81 (d, J=15.9 Hz, 1H), 3.63-3.58 (m, 2H), 3.50-3.44 (m, 2H), 3.09-2.95 (m, 4H), 2.14-2.02 (m, 2H), 1.92-1.55 (m, 10H).

UPLC-MS (basic method, 2 min) rt=1.19 min, m/z=488.2 [M+H]+.

### N-(5-cyclopentyl-1H-pyrazol-3-yl)-2-[4-(methylaminomethyl)-2-azabicyclo[2.1.1]hexan-2-yl]pyrimidin-4-amine

To a solution of benzyl N-[[2-[4-[(5-cyclopentyl-1H-pyrazol-3-yl)amino]pyrimidin-2-yl]-2-azabicyclo[2.1.1]hexan-4-yl]methyl]-N-methyl-carbamate (115 mg, 0.235 mmol) in MeOH (3 mL) under nitrogen was added 10% w/w palladium on carbon (30 mg, 0.28 mmol). The atmosphere was exchanged for hydrogen and the reaction mixture was stirred at ambient temperature for 4 hours. The reaction mixture was filtered through a pad of celite, washed with EtOAc (10 mL) and concentrated to dryness under reduced pressure. The crude residue was purified by automated flash column chromatography, eluting with a gradient of 2 M NH₃ in MeOH (0 to 20%; v/v) in CH₂Cl₂ to afford the title compound as a solid (50 mg, 0.140 mmol, 59%).

¹H NMR (400 MHz, DMSO-d₆) δ 11.90 (s, 1H), 9.33 (s, 1H), 7.84 (d, J=5.7 Hz, 1H), 6.28 (s, 2H), 4.69 (s, 1H), 3.36 (s, 2H), 3.02 (p, J=7.9 Hz, 1H), 2.90 (s, 2H), 2.40 (s, 3H), 2.08-1.95 (m, 2H), 1.90-1.80 (m, 2H), 1.75-1.68 (m, 2H), 1.65-1.51 (m, 4H), 1.40 (dd, J=4.3, 1.8 Hz, 2H).

UPLC-MS (basic method, 4 min): rt=1.28 min, m/z=354.1 [M+H]+.

### Example 31. Synthesis of Compound 32

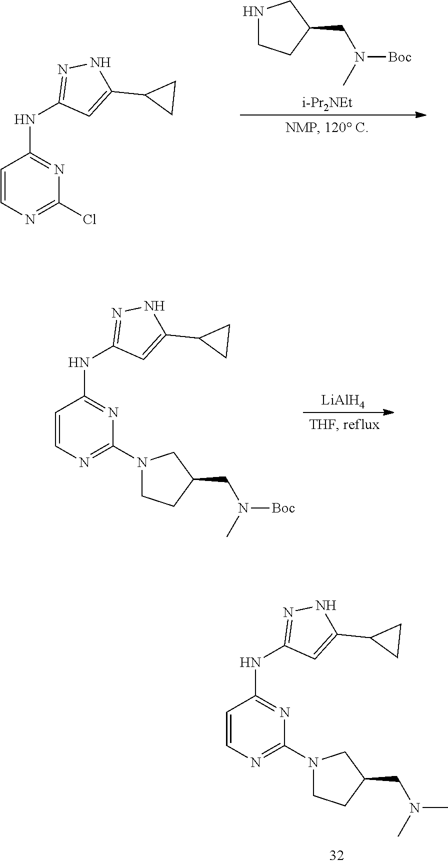 [SMILES: "i-Pr2NEt" ; Clc1nccc(Nc2cc(C3CC3)[nH]n2)n1 ; CN(C[C@H]1CCNC1)C(=O)OC(C)(C)C ; CN(C[C@H]1CCN(c2nccc(Nc3cc(C4CC4)[nH]n3)n2)C1)C(=O)OC(C)(C)C ; "LiAlH4" ; CN(C)C[C@H]1CCN(c2nccc(Nc3cc(C4CC4)[nH]n3)n2)C1]

### tert-butyl N-methyl-N-[[rac-(3S)-1-[4-[(5-cyclopropyl-1H-pyrazol-3-yl)amino]pyrimidin-2-yl]pyrrolidin-3-yl]methyl]carbamate

A solution of 2-chloro-N-(5-cyclopropyl-1H-pyrazol-3-yl)pyrimidin-4-amine (275 mg, 1.17 mmol), tert-butyl (S)-methyl(pyrrolidin-3-ylmethyl)carbamate (250 mg, 1.17 mmol) and N,N-diisopropylethylamine (0.51 mL, 2.93 mmol) in NMP (6 mL) was heated to 120° C. and stirred for 48 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as a foam (371 mg, 0.798 mmol, 68%).

¹H NMR (400 MHz, CDCl₃) δ 7.97 (d, J=3.9 Hz, 1H), 6.02 (s, 1H), 5.88 (s, 1H), 3.83-3.64 (m, 2H), 3.64-3.48 (m, 1H), 3.48-3.09 (m, 3H), 2.90, (s, 3H), 2.68-2.51 (m, 1H), 2.07 (dt, J=12.3, 6.4 Hz, 1H), 1.86 (tt, J=8.5, 5.0 Hz, 1H), 1.81-1.65 (m, 1H), 1.43 (s, 9H), 0.98-0.88 (m, 2H), 0.77-0.68 (m, 2H). 2H).

UPLC-MS (basic method, 2 min) rt=1.06 min, m/z=414.3 [M+H]+.

### N-(5-cyclopropyl-1H-pyrazol-3-yl)-2-[rac-(3R)-3-[(dimethylamino)methyl]pyrrolidin-1-yl]pyrimidin-4-amine

To a solution of tert-butyl N-methyl-N-[[rac-(3S)-1-[4-[(5-cyclopropyl-1H-pyrazol-3-yl)amino]pyrimidin-2-yl]pyrrolidin-3-yl]methyl]carbamate (154 mg, 0.331 mmol) in anhydrous THE (3 mL) was added a 2.4 M solution of lithium aluminum hydride in THE (1.34 mL, 3.22 mmol) dropwise at 0° C. The reaction mixture was heated to reflux and stirred for 3 hours. The reaction mixture was then cooled to 0° C., then quenched with water (0.13 mL), then a 10% w/v sodium hydroxide solution (0.13 mL), followed by water (0.5 mL). The reaction mixture was warmed to ambient temperature and stirred for 15 minutes, after which magnesium sulfate (ca. 1 g) was added and the reaction mixture was stirred for a further 15 minutes. The reaction mixture was filtered, and washed with EtOAc (20 mL). The eluent was then concentrated to dryness under reduced pressure. The residue was purified by reversed-phase basic prepHPLC, to afford the title compound as a solid (86 mg, 0.256 mmol, 77%).

¹H NMR (400 MHz, DMSO-d₆) δ 11.89 (s, 1H), 9.30 (s, 1H), 7.82 (d, J=5.7 Hz, 1H), 6.25 (s, 1H), 6.18 (s, 1H), 3.73-3.48 (m, 2H), 3.47-3.35 (m, 1H), 3.22-3.08 (m, 1H), 2.48-2.37 (m, 1H), 2.24 (d, J=7.7 Hz, 2H), 2.17 (s, 6H), 2.09-1.94 (m, 1H), 1.84 (ddd, J=13.2, 8.4, 5.0 Hz, 1H), 1.70-1.49 (m, 1H), 0.98-0.82 (m, 2H), 0.69-0.56 (m, 2H).

UPLC-MS (basic method, 4 min): rt=1.33 min, m/z=328.1 [M+H]+.

### Example 32. Synthesis of Compound 33

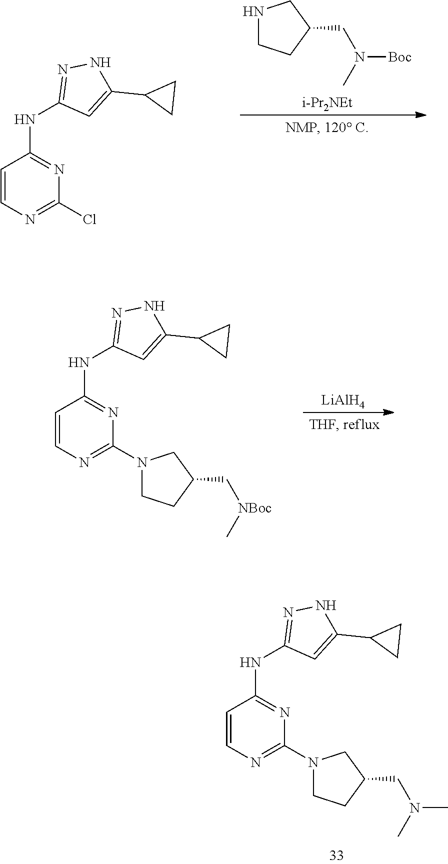 [SMILES: "i-Pr2NEt" ; Clc1nccc(Nc2cc(C3CC3)[nH]n2)n1 ; CN(C[C@@H]1CCNC1)C(=O)OC(C)(C)C ; CN(C[C@@H]1CCN(c2nccc(Nc3cc(C4CC4)[nH]n3)n2)C1)C(=O)OC(C)(C)C ; "LiAlH4" ; CN(C)C[C@@H]1CCN(c2nccc(Nc3cc(C4CC4)[nH]n3)n2)C1]

### tert-Butyl N-Methyl-N-[[rac-(3R)-1-[4-[(5-cyclopropyl-1H-pyrazol-3-yl)amino]pyrimidin-2-yl]pyrrolidin-3-yl]methyl]carbamate

A solution of 2-chloro-N-(5-cyclopropyl-1H-pyrazol-3-yl)pyrimidin-4-amine (250 mg, 1.06 mmol), tert-butyl N-methyl-N-[[(3R)-pyrrolidin-3-yl]methyl]carbamate (250 mg, 1.17 mmol) and N,N-diisopropylethylamine (0.18 mL, 1.06 mmol) in NMP (5 mL) was heated to 120° C. and stirred for 24 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude was purified by automated flash column chromatography, over silica gel, eluting with a gradient of CH₂Cl₂ to 1:9 2 M NH₃ in MeOH:CH₂Cl₂ to afford the title compound as a solid (192 mg, 0.464 mmol, 44%).

¹H NMR (400 MHz, DMSO-d₆) δ 11.87 (s, 1H), 9.37 (s, 1H), 7.79 (dd, J=5.8, 3.6 Hz, 1H), 6.17 (s, 1H), 3.60-3.38 (m, 3H), 2.78 (s, 3H), 2.03-1.91 (m, 1H), 1.81 (tt, J=8.4, 5.1 Hz, 1H), 1.68-1.57 (m, 1H), 1.40-1.27 (m, 9H), 1.22-1.12 (m, 1H), 0.92-0.83 (m, 2H), 0.60 (dt, J=6.4, 3.2 Hz, 2H).

UPLC-MS (basic method, 2 min) Rt=1.05 min, m/z=414.2 [M+H]⁺.

### N-(5-Cyclopropyl-1H-pyrazol-3-yl)-2-[rac-(3S)-3-[(dimethylamino)methyl]pyrrolidin-1-yl]pyrimidin-4-amine

To a solution of tert-butyl N-methyl-N-[[rac-(3R)-1-[4-[(5-cyclopropyl-1H-pyrazol-3-yl)amino]pyrimidin-2-yl]pyrrolidin-3-yl]methyl]carbamate (120 mg, 0.290 mmol) in anhydrous THF (10 mL) was added a 2.4 M solution of lithium aluminum hydride in THF (1.2 mL, 2.9 mmol) dropwise at 0° C. The reaction mixture was heated to reflux and stirred for 4.5 hours. The reaction mixture was then cooled to 0° C., then quenched with water (1.0 mL), then a 10% w/v sodium hydroxide solution (1.0 mL), followed by water (1.0 mL). The reaction mixture was warmed to ambient temperature and stirred for 5 minutes, after which magnesium sulfate (ca. 1 g) was added and the reaction mixture was stirred for a further 5 minutes. The reaction mixture was filtered, and the filter cake was washed with EtOAc (20 mL). The eluent was then concentrated to dryness under reduced pressure to afford the title compound as a solid (50 mg, 0.153 mmol, 53%).

¹H NMR (400 MHz, DMSO-d₆) δ 11.88 (s, 1H), 9.29 (s, 1H), 7.82 (s, 1H), 6.38-6.08 (m, 2H), 3.69-3.52 (m, 2H), 3.38 (s, 1H), 3.20-3.11 (m, 1H), 2.46-2.38 (m, 1H), 2.24 (d, J=6.9 Hz, 2H), 2.17 (s, 6H), 2.01 (s, 1H), 1.84 (s, 1H), 1.63 (s, 1H), 0.90 (d, J=8.1 Hz, 2H), 0.64 (s, 2H).

UPLC-MS (basic method, 4 min): Rt=1.35 min, m/z=328.1 [M+H]⁺.

### Example 33. Synthesis of Compound 35

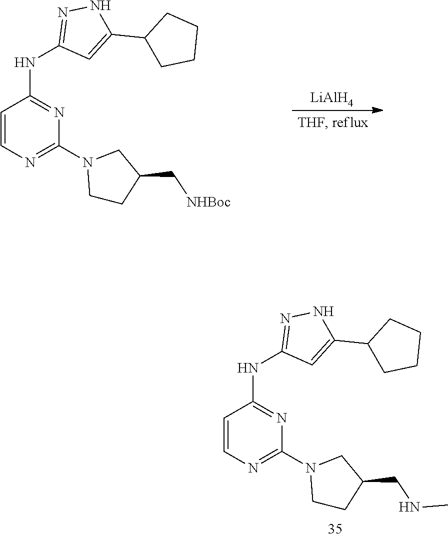 [SMILES: CC(C)(C)OC(=O)NC[C@H]1CCN(c2nccc(Nc3cc(C4CCCC4)[nH]n3)n2)C1 ; "LiAlH4" ; CNC[C@H]1CCN(c2nccc(Nc3cc(C4CCCC4)[nH]n3)n2)C1]

### N-(5-cyclopentyl-1H-pyrazol-3-yl)-2-[(3R)-3-(methylaminomethyl)pyrrolidin-1-yl]pyrimidin-4-amine

To a solution of tert-butyl N-[[(3R)-1-[4-[(5-cyclopentyl-1H-pyrazol-3-yl)amino]pyrimidin-2-yl]pyrrolidin-3-yl]methyl]carbamate (150 mg, 0.263 mmol) in anhydrous THF (2.6 mL) was added a 2.4 M solution of lithium aluminum hydride in THF (1.1 mL, 2.6 mmol) dropwise at 0° C. The reaction mixture was heated to reflux and stirred for 3 hours. The reaction mixture was then cooled to 0° C., then quenched with water (0.11 mL), then a 10% w/v sodium hydroxide solution (0.11 mL), followed by water (0.35 mL). The reaction mixture was warmed to ambient temperature and stirred for 15 minutes, after which magnesium sulfate (ca. 1 g) was added and the reaction mixture was stirred for a further 15 minutes. The reaction mixture was filtered, and washed with EtOAc (10 mL). The eluent was then concentrated to dryness under reduced pressure. The residue was purified by reversed-phase basic prepHPLC, to afford the title compound as a solid (28 mg, 81 μmol, 32%).

¹H NMR (400 MHz, DMSO-d₆) δ 11.85 (s, 1H), 9.32 (s, 1H), 7.82 (d, J=5.6 Hz, 1H), 6.40 (s, 1H), 6.18 (s, 1H), 3.70-3.61 (m, 1H), 3.57 (ddd, J=11.7, 8.1, 4.1 Hz, 1H), 3.39 (d, J=9.0 Hz, 1H), 3.21-3.10 (m, 1H), 3.00 (p, J=7.8 Hz, 1H), 2.39-2.32 (m, 1H), 2.30 (s, 3H), 2.09-1.91 (m, 4H), 1.76-1.51 (m, 8H).

UPLC-MS (basic method, 4 min): rt=1.34 min, m/z=342.1 [M+H]⁺.

### Example 34. Synthesis of Compound 37

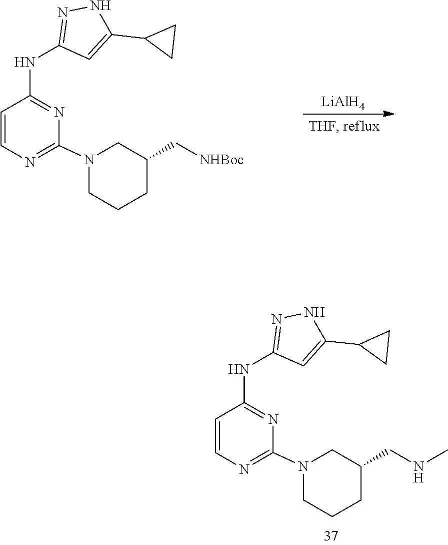 [SMILES: CC(C)(C)OC(=O)NC[C@@H]1CCCN(c2nccc(Nc3cc(C4CC4)[nH]n3)n2)C1 ; "LiAlH4" ; CNC[C@@H]1CCCN(c2nccc(Nc3cc(C4CC4)[nH]n3)n2)C1]

### N-(5-cyclopropyl-1H-pyrazol-3-yl)-2-[(3S)-3-(methylaminomethyl)-1-piperidyl]pyrimidin-4-amine

To a solution of tert-butyl N-[[(3S)-1-[4-[(5-cyclopropyl-1H-pyrazol-3-yl)amino]pyrimidin-2-yl]-3-piperidyl]methyl]carbamate (150 mg, 0.363 mmol) in anhydrous THF (4 mL) was added a 2.4 M solution of lithium aluminum hydride in THF (1.5 mL, 3.6 mmol) dropwise at 0° C. The reaction mixture was warmed to ambient temperature, then stirred for 1 hour. The reaction mixture was then heated to reflux and stirred for 3 hours. The reaction mixture was then cooled to 0° C., then quenched with water (0.15 mL), then a 10% w/v sodium hydroxide solution (0.15 mL), followed by water (0.5 mL). The reaction mixture was warmed to ambient temperature and stirred for 15 minutes, after which magnesium sulfate (ca. 1 g) was added and the reaction mixture was stirred for a further 15 minutes. The reaction mixture was filtered, and washed with EtOAc (20 mL). The eluent was then concentrated to dryness under reduced pressure. The residue was purified by reversed-phase basic prepHPLC, to afford the title compound as a solid (43 mg, 0.130 mmol, 36%).

¹H NMR (400 MHz, DMSO-d₆) δ 11.93 (s, 1H), 9.37 (s, 1H), 7.83 (d, J=5.7 Hz, 1H), 6.12 (s, 2H), 4.76-4.24 (m, 2H), 2.88 (t, J=11.1 Hz, 1H), 2.63 (t, J=11.6 Hz, 1H), 2.36 (d, J=6.7 Hz, 2H), 2.27 (s, 3H), 1.90-1.77 (m, 2H), 1.68-1.52 (m, 2H), 1.43-1.30 (m, 1H), 1.16 (qd, J=11.8, 3.9 Hz, 1H), 0.94-0.87 (m, 2H), 0.70-0.64 (m, 2H).

UPLC-MS (basic method, 4 min): rt=1.30 min, m/z=328.1 [M+H]+.

### Example 35. Synthesis of Compound 38

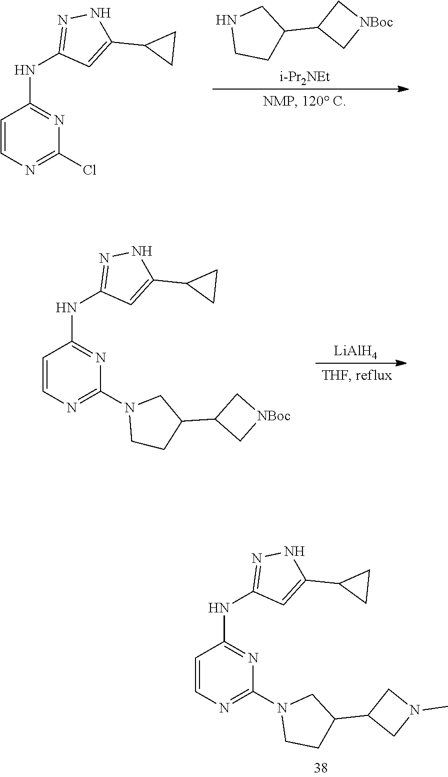 [SMILES: "i-Pr2NEt" ; Clc1nccc(Nc2cc(C3CC3)[nH]n2)n1 ; CC(C)(C)OC(=O)N1CC(C2CCNC2)C1 ; CC(C)(C)OC(=O)N1CC(C2CCN(c3nccc(Nc4cc(C5CC5)[nH]n4)n3)C2)C1 ; "LiAlH4" ; CN1CC(C2CCN(c3nccc(Nc4cc(C5CC5)[nH]n4)n3)C2)C1]

### tert-Butyl 3-[1-[4-[(5-Cyclopropyl-1H-pyrazol-3-yl)amino]pyrimidin-2-yl]pyrrolidin-3-yl]azetidine-1-carboxylate

A solution of 2-chloro-N-(5-cyclopropyl-1H-pyrazol-3-yl)pyrimidin-4-amine (348 mg, 1.48 mmol), tert-butyl 3-(pyrrolidin-3-yl)azetidine-1-carboxylate (501 mg, 2.22 mmol) and N,N-diisopropylethylamine (0.64 mL, 3.7 mmol) in NMP (5 mL) was heated to 120° C. and stirred for 24 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude residue was purified by automated flash column chromatography, eluting with a gradient of CH₂Cl₂ to 1:9 2 M NH₃ in MeOH:CH₂Cl₂ to afford the title compound as an oil (365 mg, 0.849 mmol, 58%).

¹H NMR (400 MHz, DMSO-d₆) δ 11.98-11.81 (m, 1H), 9.33 (s, 1H), 7.82 (d, J=5.7 Hz, 1H), 6.22 (s, 2H), 3.93 (s, 2H), 3.80-3.53 (m, 4H), 3.41 (d, J=9.4 Hz, 1H), 3.04 (dd, J=10.9, 6.9 Hz, 1H), 2.09-1.96 (m, 1H), 1.92-1.80 (m, 1H), 1.66-1.51 (m, 1H), 1.44-1.32 (m, 11H), 0.91 (dd, J=8.1, 2.9 Hz, 2H), 0.63 (td, J=6.0, 3.8 Hz, 2H).

UPLC-MS (basic method, 2 min): Rt: 1.06 min, m/z: 426.5 [M+H]⁺.

### N-(5-Cyclopropyl-1H-pyrazol-3-yl)-2-[3-(1-methylazetidin-3-yl)pyrrolidin-1-yl]pyrimidin-4-amine

To a solution of tert-butyl 3-[1-[4-[(5-cyclopropyl-1H-pyrazol-3-yl)amino]pyrimidin-2-yl]pyrrolidin-3-yl]azetidine-1-carboxylate (360 mg, 0.85 mmol) in anhydrous THE (12 mL) was added a 2.4 M solution of lithium aluminum hydride in THE (3.5 mL, 8.5 mmol) dropwise at 0° C. The reaction mixture was heated to reflux and stirred for 2 hours. The reaction mixture was then cooled to 0° C., then quenched with water (3.2 mL), then a 15% w/v sodium hydroxide solution (3.2 mL), followed by water (9.6 mL). The reaction mixture was warmed to ambient temperature and stirred for 15 minutes, after which magnesium sulfate (ca. 1 g) was added, and the reaction mixture was stirred for a further 15 minutes. The reaction mixture was passed through a pad of celite, and the filter cake was washed with EtOAc (20 mL). The eluent was concentrated to dryness under reduced pressure. 50 mg of the crude residue was loaded onto an SCX cartridge, the cartridge was then washed with methanol, then the compound was eluted with 2 M NH₃ in methanol, and the eluent was concentrated to dryness under reduced pressure to afford the title compound as a solid (30 mg, 88 μmol, 10%).

¹H NMR (400 MHz, DMSO-d₆) δ 11.86 (s, 1H), 9.34 (s, 1H), 7.82 (d, J=5.7 Hz, 1H), 6.20 (s, 2H), 3.67-3.53 (m, 2H), 3.49-3.41 (m, 2H), 3.41-3.35 (m, 1H), 3.14-2.96 (m, 3H), 2.47-2.42 (m, 2H), 2.34 (s, 3H), 2.04-1.94 (m, 1H), 1.85 (tt, J=8.4, 5.1 Hz, 1H), 1.59-1.49 (m, 1H), 0.96-0.81 (m, 2H), 0.67-0.54 (m, 2H).

UPLC-MS (basic method, 4 min): Rt: 1.17 min, m/z: 340.1 [M+H]⁺.

### Example 36. Synthesis of Compound 42

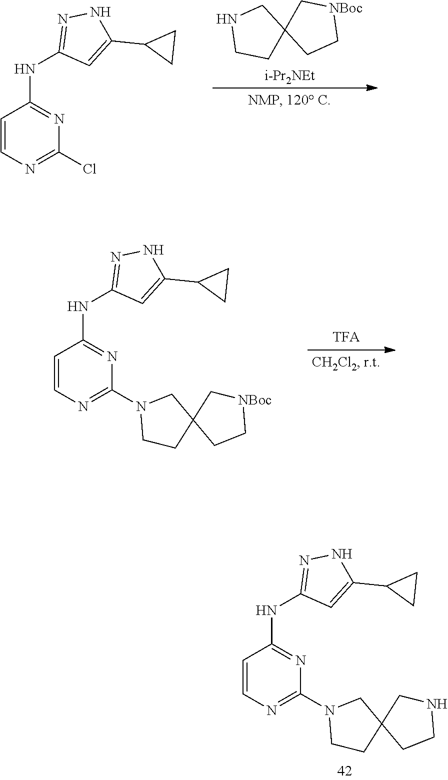 [SMILES: "i-Pr2NEt" ; Clc1nccc(Nc2cc(C3CC3)[nH]n2)n1 ; CC(C)(C)OC(=O)N1CCC2(CCNC2)C1 ; CC(C)(C)OC(=O)N1CCC2(CCN(c3nccc(Nc4cc(C5CC5)[nH]n4)n3)C2)C1 ; c1cc(Nc2cc(C3CC3)[nH]n2)nc(N2CCC3(CCNC3)C2)n1]

### (±)-tert-Butyl 7-[4-[(5-Cyclopropyl-1H-pyrazol-3-yl)amino]pyrimidin-2-yl]-2,7-diazaspiro[4.4]nonane-2-carboxylate

A solution of 2-chloro-N-(5-cyclopropyl-1H-pyrazol-3-yl)pyrimidin-4-amine (700 mg, 2.97 mmol), (±)-tert-butyl 2,7-diazaspiro[4.4]nonane-2-carboxylate (498 mg, 1.10 mmol) and N,N-diisopropylethylamine (1.3 mL, 7.4 mmol) in NMP (7 mL) was heated to 120° C. and stirred for 18 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (1.10 g, 2.59 mmol, 87%).

¹H NMR (400 MHz, DMSO-d₆) δ 11.92 (s, 1H), 9.29 (s, 1H), 7.85 (d, J=5.7 Hz, 1H), 6.92-6.82 (m, 1H), 6.24 (s, 1H), 6.09 (s, 1H), 4.53-4.37 (m, 2H), 2.94-2.80 (m, 3H), 2.66-2.55 (m, 1H), 1.88 (tt, J=8.2, 5.2 Hz, 1H), 1.80-1.72 (m, 1H), 1.71-1.61 (m, 1H), 1.61-1.49 (m, 1H), 1.43-1.29 (m, 10H), 1.24-1.10 (m, 1H), 0.96-0.87 (m, 2H), 0.66 (dt, J=6.6, 3.3 Hz, 2H).

UPLC-MS (basic method, 2 min): Rt: 1.05 min, m/z: 426.5 [M+H]⁺.

### N-(5-Cyclopropyl-1H-pyrazol-3-yl)-2-(2,7-diazaspiro[4.4]nonan-2-yl)pyrimidin-4-amine

To a solution of tert-butyl 7-[4-[(5-cyclopropyl-1H-pyrazol-3-yl)amino]pyrimidin-2-yl]-2,7-diazaspiro[4.4]nonane-2-carboxylate (550 mg, 0.48 mmol) in CH₂Cl₂ (10 mL) was added trifluoroacetic acid (10 mL, 130 mmol) at ambient temperature and the reaction was stirred for 30 minutes. The reaction mixture was then concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure to afford the title compound as a solid (359 mg, 1.10 mmol, 85%).

¹H NMR (400 MHz, DMSO-d₆) δ 11.87 (s, 1H), 9.32 (s, 1H), 7.82 (d, J=5.7 Hz, 1H), 6.34-6.00 (m, 2H), 3.60-3.38 (m, 4H), 2.85 (t, J=7.0 Hz, 2H), 2.67 (s, 2H), 1.96-1.78 (m, 3H), 1.67 (dt, J=8.1, 3.8 Hz, 2H), 0.95-0.86 (m, 2H), 0.71-0.60 (m, 2H).

¹H NMR (400 MHz, methanol-d₄) δ 7.84 (d, J=5.9 Hz, 1H), 6.28-5.98 (m, 2H), 3.71-3.56 (m, 2H), 3.56-3.43 (m, 2H), 3.03 (t, J=7.1 Hz, 2H), 2.92-2.79 (m, 2H), 2.01 (t, J=7.0 Hz, 2H), 1.95-1.78 (m, 3H), 1.01-0.92 (m, 2H), 0.76-0.67 (m, 2H).

UPLC-MS (basic method, 4 min): Rt: 1.10 min, m/z: 326.1 [M+H]⁺.

### Example 37. Synthesis of Compound 41

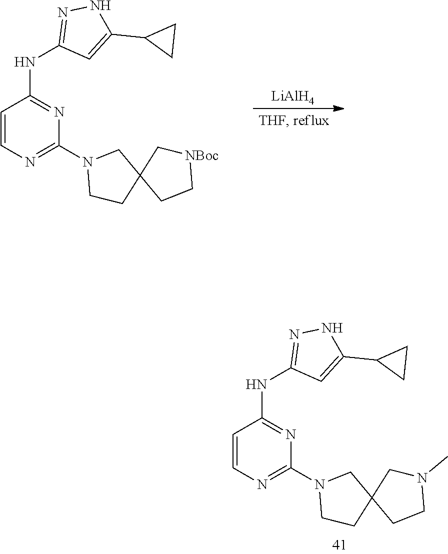 [SMILES: CC(C)(C)OC(=O)N1CCC2(CCN(c3nccc(Nc4cc(C5CC5)[nH]n4)n3)C2)C1 ; "LiAlH4" ; CN1CCC2(CCN(c3nccc(Nc4cc(C5CC5)[nH]n4)n3)C2)C1]

### N-(5-Cyclopropyl-1H-pyrazol-3-yl)-2-(2,7-diazaspiro[4.4]nonan-2-yl)pyrimidin-4-amine

To a solution of tert-butyl 7-[4-[(5-cyclopropyl-1H-pyrazol-3-yl)amino]pyrimidin-2-yl]-2,7-diazaspiro[4.4]nonane-2-carboxylate (550 mg, 0.741 mmol) in anhydrous THF (20 mL) was added a 2.4 M solution of lithium aluminum hydride in THF (5.4 mL, 13 mmol) dropwise at 0° C. The reaction mixture was heated to reflux and stirred for 3 hours. The reaction mixture was then cooled to 0° C., then quenched with water (500 μL), then a 15% w/v sodium hydroxide solution (500 μL), followed by water (1.5 mL). The reaction mixture was warmed to ambient temperature and stirred for 15 minutes, after which magnesium sulfate (ca. 2 g) was added, and the reaction mixture was stirred for a further 15 minutes. The reaction mixture was passed through a pad of celite, and the filter cake was washed with CH₂Cl₂ (20 mL). The eluent was then concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced. 50 mg of the crude material was purified by reversed-phase basic prepHPLC to afford the title compound as a solid (34 mg, 0.10 mmol, 8%).

¹H NMR (400 MHz, DMSO-d₆) δ 11.87 (s, 1H), 9.31 (s, 1H), 7.82 (d, J=5.7 Hz, 1H), 6.42-6.00 (m, 2H), 3.53-3.32 (m, 4H), 2.58-2.52 (m, 2H), 2.45-2.35 (m, 2H), 2.23 (s, 3H), 1.99-1.81 (m, 3H), 1.75 (t, J=7.0 Hz, 2H), 0.95-0.83 (m, 2H), 0.69-0.60 (m, 2H)

¹H NMR (400 MHz, methanol-d₄) δ 7.80 (d, J=5.9 Hz, 1H), 6.35-5.96 (m, 2H), 3.70-3.40 (m, 4H), 2.76-2.50 (m, 4H), 2.35 (s, 2H), 2.10-1.92 (m, 2H), 1.92-1.81 (m, 3H), 0.97-0.90 (m, 2H), 0.74-0.65 (m, 2H).

UPLC-MS (basic method, 4 min): Rt: 1.20 min, m/z: 340.2 [M+H]⁺.

### Example 38. Synthesis of Compound 43

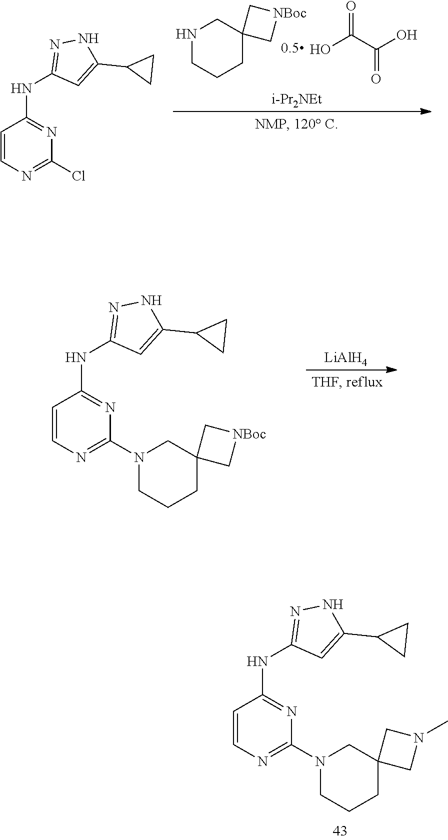

### tert-Butyl 8-[4-[(5-Cyclopropyl-1H-pyrazol-3-yl)amino]pyrimidin-2-yl]-2,8-diazaspiro[3.5]nonane-2-carboxylate

A solution of 2-chloro-N-(5-cyclopropyl-1H-pyrazol-3-yl)pyrimidin-4-amine (360 mg, 1.53 mmol), tert-butyl 2,6-diazaspiro[3.5]nonane-2-carboxylate hemioxalate (497 mg, 1.83 mmol) and N,N-diisopropylethylamine (0.85 mL, 4.9 mmol) in NMP (5 mL) was heated to 120° C. and stirred for 24 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude residue was purified by automated flash column chromatography, eluting with a gradient of CH₂Cl₂ to 1:9 2 M NH₃ in MeOH:CH₂Cl₂ to afford the title compound as a solid (120 mg, 68% pure by UPLC-MS, 0.192 mmol, 13%).

¹H NMR (400 MHz, methanol-d₄) δ 7.81 (d, J=6.0 Hz, 1H), 6.11 (s, 2H), 3.67-3.51 (m, 5H), 3.26 (s, 4H), 1.90-1.76 (m, 2H), 1.57-1.51 (m, 2H), 1.39 (s, 9H), 0.98-0.87 (m, 2H), 0.74-0.62 (m, 2H).

UPLC-MS (basic method, 2 min): Rt: 1.11 min, m/z: 426.3 [M+H]⁺.

### N-(5-Cyclopropyl-1H-pyrazol-3-yl)-2-[rac-(3aR,6aR)-2,3,3a,4,6,6a-hexahydro-1H-pyrrolo[3,4-c]pyrrol-5-yl]pyrimidin-4-amine

To a solution of tert-butyl 8-[4-[(5-cyclopropyl-1H-pyrazol-3-yl)amino]pyrimidin-2-yl]-2,8-diazaspiro[3.5]nonane-2-carboxylate (100 mg, 0.24 mmol) in anhydrous THF (10 mL) was added a 2.4 M solution of lithium aluminum hydride in THF (1.0 mL, 2.4 mmol) dropwise at 0° C. The reaction mixture was heated to reflux and stirred for 2 hours. The reaction mixture was then cooled to 0° C., then quenched with water (900 μL), then a 15% w/v sodium hydroxide solution (900 μL), followed by water (2.7 mL). The reaction mixture was warmed to ambient temperature and stirred for 15 minutes, after which magnesium sulfate (ca. 1 g) was added, and the reaction mixture was stirred for a further 15 minutes. The reaction mixture was passed through a pad of celite, and the filter cake was washed with EtOAc (20 mL). The eluent was then concentrated to dryness under reduced pressure. The crude residue was purified by reversed-phase prepHPLC to afford the title compound as a solid (22 mg, 65 μmol, 28%).

¹H NMR (400 MHz, methanol-d₄) δ 7.88 (d, J=5.9 Hz, 1H), 6.20 (s, 1H), 6.06 (s, 1H), 3.90 (s, 2H), 3.75-3.61 (m, 2H), 3.43 (d, J=8.8 Hz, 2H), 3.35 (s, 2H), 2.57 (s, 3H), 1.94-1.82 (m, 3H), 1.61-1.54 (m, 2H), 1.08-0.93 (m, 2H), 0.79-0.67 (m, 2H).

UPLC-MS (basic method, 4 min): Rt: 1.36 min, m/z: 340.3 [M+H]⁺.

### Example 39. Synthesis of Compound 50

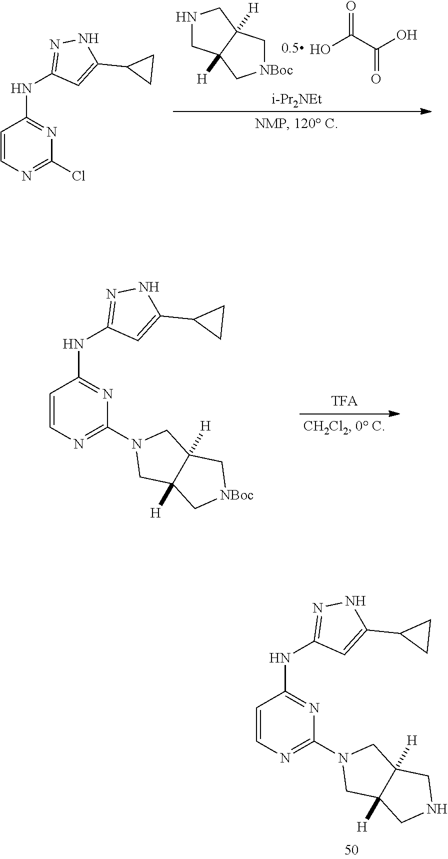

### tert-Butyl rac-(3aS,6aS)-2-[4-[(5-Cyclopropyl-1H-pyrazol-3-yl)amino]pyrimidin-2-yl]-1,3,3a,4,6,6a-hexahydropyrrolo[3,4-c]pyrrole-5-carboxylate

A solution of 2-chloro-N-(5-cyclopropyl-1H-pyrazol-3-yl)pyrimidin-4-amine (415 mg, 1.76 mmol), (±)-tert-butyl (3aS,6aS)-octahydropyrrolo[3,4-c]pyrrole-2-carboxylate hemioxalate (498 mg, 1.10 mmol) and N,N-diisopropylethylamine (1.1 mL, 6.1 mmol) in NMP (6 mL) was heated to 120° C. and stirred for 48 hours. A further addition of N,N-diisopropylethylamine (1.1 mL, 6.1 mmol) was made and the reaction was heated to 120° C. and stirred for a further 24 h. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (559 mg, 1.36 mmol, 77%).

¹H NMR (400 MHz, chloroform-d) δ 8.01 (d, J=5.7 Hz, 1H), 6.94 (s, 1H), 6.07 (s, 1H), 5.85 (s, 1H), 4.00-3.88 (m, 2H), 3.74 (dd, J=9.9, 5.9 Hz, 1H), 3.67 (dd, J=9.7, 6.1 Hz, 1H), 3.32-3.16 (m, 2H), 3.09 (td, J=10.2, 3.4 Hz, 2H), 2.38-2.21 (m, 2H), 1.87 (tt, J=8.4, 5.1 Hz, 1H), 1.48 (s, 9H), 1.02-0.90 (m, 2H), 0.77-0.68 (m, 2H).

UPLC-MS (basic method, 2 min): Rt: 1.04 min, m/z: 412.4 [M+H]⁺.

### N-(5-Cyclopropyl-1H-pyrazol-3-yl)-2-[rac-(3aR,6aR)-2,3,3a,4,6,6a-hexahydro-1H-pyrrolo[3,4-c]pyrrol-5-yl]pyrimidin-4-amine

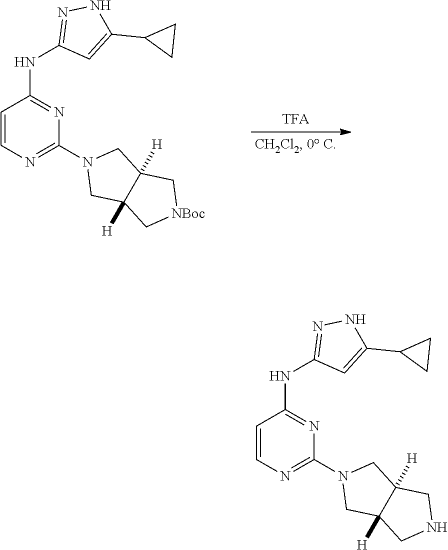 [SMILES: CC(C)(C)OC(=O)N1C[C@@H]2CN(c3nccc(Nc4cc(C5CC5)[nH]n4)n3)C[C@H]2C1 ; c1cc(Nc2cc(C3CC3)[nH]n2)nc(N2C[C@H]3CNC[C@@H]3C2)n1]

To a solution of tert-butyl rac-(3aS,6aS)-2-[4-[(5-cyclopropyl-1H-pyrazol-3-yl)amino]pyrimidin-2-yl]-1,3,3a,4,6,6a-hexahydropyrrolo[3,4-c]pyrrole-5-carboxylate (250 mg, 0.48 mmol) in CH₂Cl₂ (10 mL) was added trifluoro acetic acid (10 mL, 130 mmol) at ambient temperature and stirred for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure to afford the title compound as a solid (189 mg, 0.589 mmol, 97%).

¹H NMR (400 MHz, DMSO-d₆) δ 11.84 (s, 1H), 9.34 (s, 1H), 7.83 (d, J=5.6 Hz, 1H), 6.32-6.08 (m, 2H), 3.80-3.73 (m, 2H), 3.14-2.87 (m, 4H), 2.62-2.53 (m, 2H), 2.33-2.10 (m, 2H), 1.91-1.80 (m, 1H), 0.91-0.90 (m, 1H), 0.73-0.60 (m, 2H).

¹H NMR (400 MHz, methanol-d₄) δ 7.84 (d, J=6.0 Hz, 1H), 6.23-6.01 (m, 2H), 3.98-3.81 (m, 2H), 3.25-3.17 (m, 4H), 2.77 (t, J=10.3 Hz, 2H), 2.47-2.30 (m, 2H), 1.95-1.83 (m, 1H), 1.01-0.88 (m, 2H), 0.75-0.67 (m, 2H).

UPLC-MS (basic method, 4 min): Rt: 0.97 min, m/z: 312.0 [M+H]⁺.

### Example 40. Synthesis of Compound 47

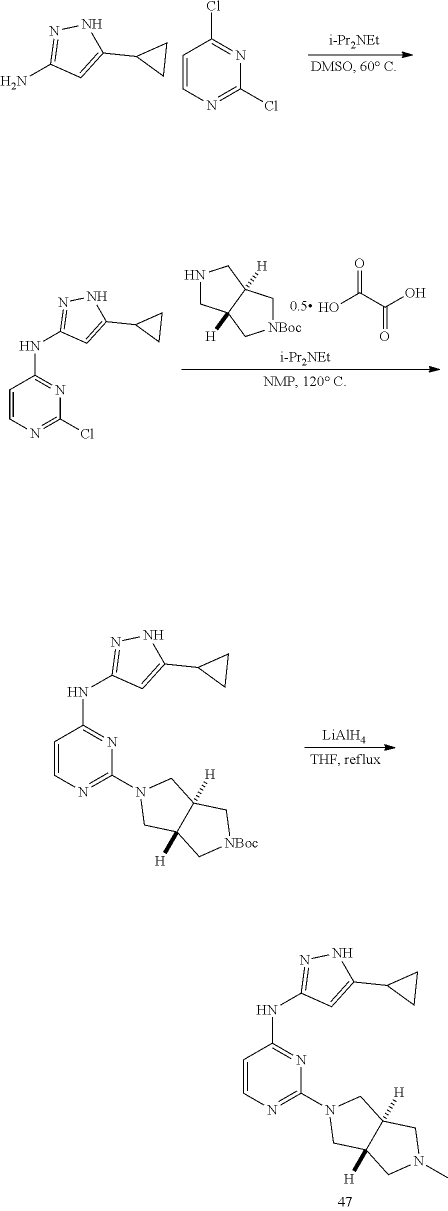

### N-(5-Cyclopropyl-1H-pyrazol-3-yl)-2-[rac-(3aR,6aR)-2-methyl-1,3,3a,4,6,6a-hexahydropyrrolo[3,4-c]pyrrol-5-yl]pyrimidin-4-amine

To a solution of tert-butyl rac-(3aS,6aS)-2-[4-[(5-cyclopropyl-1H-pyrazol-3-yl)amino]pyrimidin-2-yl]-1,3,3a,4,6,6a-hexahydropyrrolo[3,4-c]pyrrole-5-carboxylate (305 mg, 0.741 mmol) in anhydrous THF (20 mL) was added a 2.4 M solution of lithium aluminum hydride in THF (3.1 mL, 7.4 mmol) dropwise at 0° C. The reaction mixture was heated to reflux and stirred for 3 hours. The reaction mixture was then cooled to 0° C., then quenched with water (300 μL), then a 15% w/v sodium hydroxide solution (300 μL), followed by water (900 μL). The reaction mixture was warmed to ambient temperature and stirred for 15 minutes, after which magnesium sulfate (ca. 1 g) was added, and the reaction mixture was stirred for a further 15 minutes. The reaction mixture was passed through a pad of celite, and the filter cake was washes with CH₂Cl₂ (20 mL) followed by ethanol (20 mL). The eluent was then concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure to afford the title compound as a solid (115 mg, 0.353 mmol, 48%).

¹H NMR (400 MHz, DMSO-d₆) δ 11.86 (s, 1H), 9.29 (s, 1H), 7.82 (d, J=5.7 Hz, 1H), 6.43-6.01 (m, 2H), 3.79-3.69 (m, 2H), 3.15-2.97 (m, 2H), 2.83-2.74 (m, 2H), 2.60-2.52 (m, 2H), 2.44 (s, 3H), 2.32-2.18 (m, 2H), 1.86 (tt, J=8.7, 5.0 Hz, 1H), 0.93-0.87 (m, 2H), 0.69-0.60 (m, 2H).

¹H NMR (400 MHz, methanol-d₄) δ 7.83 (d, J=6.0 Hz, 1H), 6.32-6.04 (m, 2H), 3.93-3.75 (m, 2H), 3.22-3.09 (m, 2H), 2.98 (dd, J=9.4, 5.7 Hz, 2H), 2.66 (t, J=9.9 Hz, 2H), 2.56 (s, 3H), 2.47-2.35 (m, 2H), 1.89 (tt, J=8.9, 5.1 Hz, 1H), 0.99-0.92 (m, 2H), 0.78-0.67 (m, 2H).

UPLC-MS (basic method, 4 min): Rt: 1.17 min, m/z: 326.1 [M+H]⁺.

### Example 41. Synthesis of Compound 66

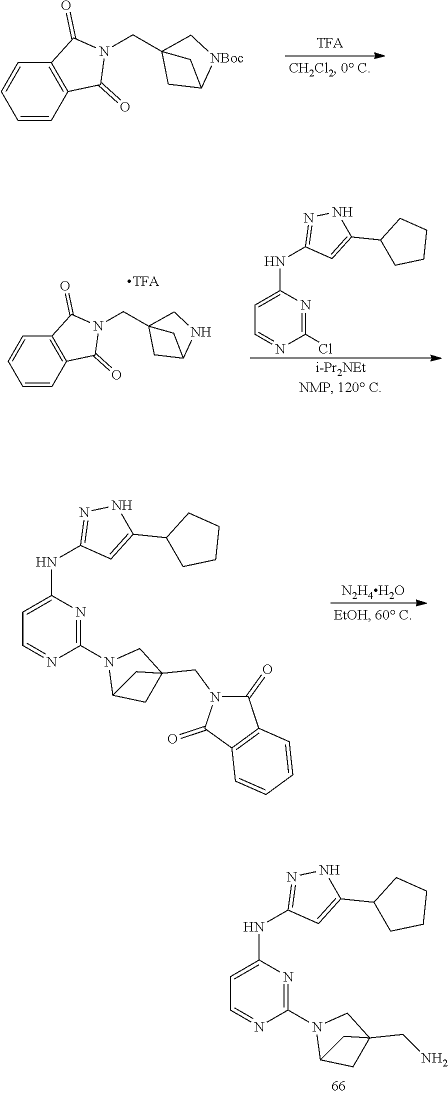

### 2-(2-Azabicyclo[2.1.1]hexan-4-ylmethyl)isoindoline-1,3-dione trifluoroacetate

A solution of tert-butyl 4-[(1,3-dioxoisoindolin-2-yl)methyl]-2-azabicyclo[2.1.1]hexane-2-carboxylate (600 mg, 1.75 mmol) in CH₂Cl₂ (20 mL) was added trifluoroacetic acid (4 mL, 52.27 mmol) and the reaction stirred at 0° C. for 30 minutes, after which the reaction mixture was concentrated to dryness under reduced pressure to afford the title compound as a solid (590 mg, 1.66 mmol, 95%).

¹H NMR (400 MHz, methanol-d₄) δ 7.92-7.85 (m, 2H), 7.85-7.77 (m, 2H), 4.12 (td, J=3.4, 2.0 Hz, 1H), 4.08-3.94 (m, 2H), 3.27-3.22 (m, 2H), 2.04 (d, J=7.0 Hz, 2H), 1.65-1.50 (m, 2H).

UPLC-MS (basic method, 2 min): Rt: 0.72 min, m z=243.1 [M+H]⁺.

### 2-[[2-[4-[(5-Cyclopentyl-1H-pyrazol-3-yl)amino]pyrimidin-2-yl]-2-azabicyclo[2.1.1]hexan-4-yl]methyl]isoindoline-1,3-dione

A solution of 2-chloro-N-(5-cyclopentyl-1H-pyrazol-3-yl)pyrimidin-4-amine (200 mg, 0.758 mmol), 2-(2-azabicyclo[2.1.1]hexan-4-ylmethyl)isoindoline-1,3-dione trifluoroacetate (220 mg, 0.910 mmol) and N,N-diisopropylethylamine (0.66 mL, 3.79 mmol) in NMP (5 mL) was heated to 120° C. and stirred for 96 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The residue was purified by normal-phase column chromatography, over silica gel, eluting with a gradient of CH₂Cl₂ to 9:1 CH₂Cl₂:0.1% NEt₃ in MeOH to afford the title compound as a solid (112 mg, 0.239 mmol, 31%).

¹H NMR (400 MHz, DMSO-d₆) δ 11.83 (s, 1H), 9.31 (s, 1H), 7.86-7.73 (m, 5H), 6.22 (s, 2H), 4.62 (t, J=1.8 Hz, 1H), 3.90 (s, 2H), 2.95 (p, J=8.0 Hz, 1H), 1.96 (d, J=10.1 Hz, 2H), 1.83-1.75 (m, 2H), 1.70 (s, 2H), 1.57 (s, 4H), 1.38 (dd, J=4.4, 1.8 Hz, 2H), 1.15 (t, J=16.9 Hz, 2H).

UPLC-MS (basic method, 2 min): Rt: 1.09 min, m/z=470.2 [M+H]⁺.

### 2-[4-(aminomethyl)-2-azabicyclo[2.1.1]hexan-2-yl]-N-(5-cyclopentyl-1H-pyrazol-3-yl)pyrimidin-4-amine

To a solution of 2-[[2-[4-[(5-cyclopentyl-1H-pyrazol-3-yl)amino]pyrimidin-2-yl]-2-azabicyclo[2.1.1]hexan-4-yl]methyl]isoindoline-1,3-dione (110 mg, 0.23 mmol) in EtOH (4 mL) was added hydrazine hydrate (34 μL, 0.35 mmol) and the reaction mixture was heated to 60° C. for one hour. The reaction mixture was cooled to room temperature, then filtered through a pad of celite. The filtrate was then concentrated to dryness under reduced pressure. The crude compound was loaded onto an SCX cartridge, the cartridge was then washed with methanol, then the compound was eluted with 2 M NH₃ in methanol, and the eluent was concentrated to dryness under reduced pressure to afford the title compound as a solid (58 mg, 0.17 mmol, 73%).

¹H NMR (400 MHz, DMSO-d₆) δ 11.84 (s, 1H), 9.28 (s, 1H), 7.79 (d, J=5.7 Hz, 1H), 6.22 (s, 2H), 4.68-4.61 (m, 1H), 2.97 (p, J=8.0 Hz, 1H), 2.78 (s, 2H), 2.00-1.90 (m, 2H), 1.77-1.71 (m, 2H), 1.70-1.48 (m, 7H), 1.33-1.26 (m, 2H).

¹H NMR (400 MHz, methanol-d₄) δ 7.84-7.75 (m, 1H), 6.33-6.05 (m, 2H), 4.77-4.74 (m, 1H), 3.37 (s, 2H), 3.10-3.00 (m, 1H), 2.95 (s, 2H), 2.12-1.97 (m, 2H), 1.93-1.83 (m, 2H), 1.80-1.57 (m, 6H), 1.51-1.40 (m, 2H).

UPLC-MS (basic method, 4 min): Rt: 1.28 min, m/z=340.1 [M+H]⁺.

### Example 42. Synthesis of Compound 102

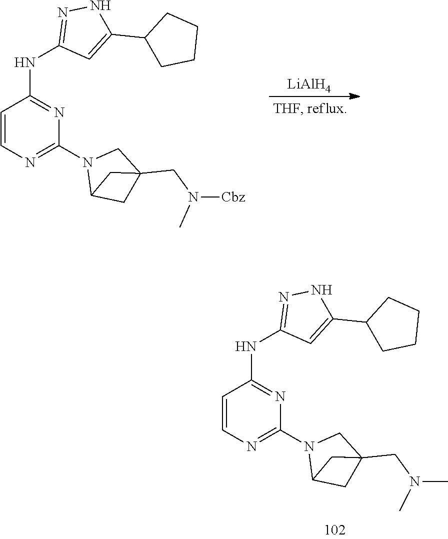 [SMILES: CN(CC12CC(C1)N(c1nccc(Nc3cc(C4CCCC4)[nH]n3)n1)C2)C(=O)OCc1ccccc1 ; "LiAlH4" ; CN(C)CC12CC(C1)N(c1nccc(Nc3cc(C4CCCC4)[nH]n3)n1)C2]

### N-(5-Cyclopentyl-1H-pyrazol-3-yl)-2-[4-(methylaminomethyl)-2-azabicyclo[2.1.1]hexan-2-yl]pyrimidin-4-amine

To a solution of benzyl N-[[2-[4-[(5-cyclopentyl-1H-pyrazol-3-yl)amino]pyrimidin-2-yl]-2-azabicyclo[2.1.1]hexan-4-yl]methyl]-N-methyl-carbamate (104 mg, 0.213 mmol) in THF (3 mL) under nitrogen was added a 2.4 M solution of lithium aluminum hydride in THF (0.89 mL, 2.1 mmol). The reaction mixture was stirred at reflux for 1 hour. The reaction mixture was then cooled to 0° C., then quenched with water (0.9 mL), then a 10% w/v sodium hydroxide solution (0.9 mL), followed by water (0.5 mL). The reaction mixture was warmed to ambient temperature and stirred for 15 minutes, after which magnesium sulfate (ca. 1 g) was added and the reaction mixture was stirred for a further 15 minutes. The reaction mixture was filtered, and the filter cake was washed with EtOAc (20 mL). The eluent was then concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure to afford the title compound as a solid (62 mg, 0.166 mmol, 78%).

¹H NMR (400 MHz, DMSO-d₆) δ 11.88 (s, 1H), 9.33 (s, 1H), 7.83 (d, J=5.7 Hz, 1H), 6.24 (s, 2H), 4.68 (t, J=1.8 Hz, 1H), 3.37 (s, 2H), 3.02 (p, J=7.9 Hz, 1H), 2.55 (s, 2H), 2.16 (s, 6H), 2.03-1.93 (m, 2H), 1.81-1.76 (m, 2H), 1.76-1.67 (m, 2H), 1.67-1.52 (m, 4H), 1.44 (dd, J=4.3, 1.8 Hz, 2H)

UPLC-MS (basic method, 4 min): rt=1.61 min, m/z=368.2 [M+H]+

### Example 43. Synthesis of Compound 84

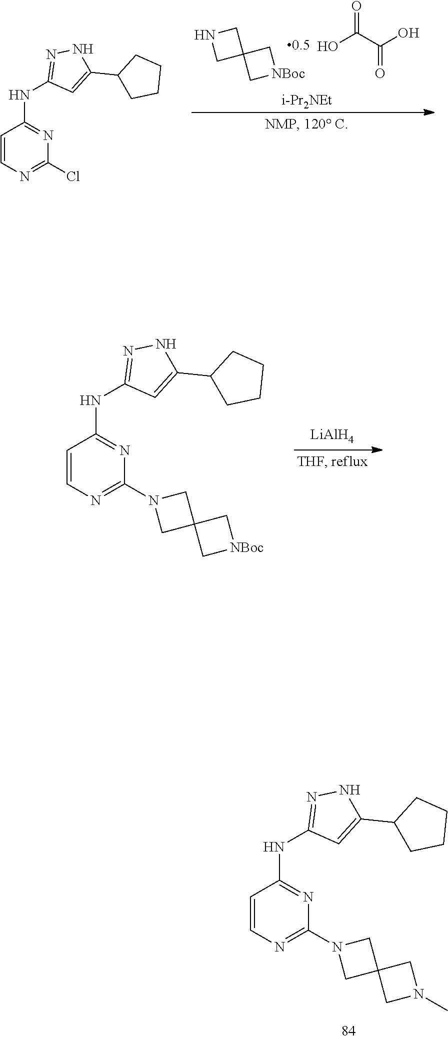

### tert-Butyl 6-[4-[(5-cyclopentyl-1H-pyrazol-3-yl)amino]pyrimidin-2-yl]-2,6-diazaspiro[3.3]heptane-2-carboxylate

A solution of 2-chloro-N-(5-cyclopentyl-1H-pyrazol-3-yl)pyrimidin-4-amine (557 mg, 2.11 mmol), tert-butyl 2,6-diazaspiro[3.3]heptane-2-carboxylate hemioxalate (507 mg, 2.08 mmol) and N,N-diisopropylethylamine (0.82 mL, 4.69 mmol) in NMP (6 mL) was heated to 120° C. and stirred for 72 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude residue was purified by automated flash column chromatography, over silica gel, eluting with a gradient of CH₂Cl₂ to 1:9 2 M NH₃ in MeOH:CH₂Cl₂, to afford the title compound as a solid (391 mg, 0.873 mmol, 84%).

¹H NMR (400 MHz, DMSO-d₆) δ 7.98 (d, J=5.7 Hz, 1H), 6.29 (s, 1H), 6.12 (s, 1H), 4.25 (s, 4H), 4.11 (s, 4H), 3.71-3.60 (m, 2H), 3.11-3.01 (m, 1H), 2.15-2.04 (m, 2H), 1.86-1.53 (m, 4H), 1.45 (s, 9H).

UPLC-MS (basic method, 2 min): Rt: 1.09 min, m/z: 426.2 [M+H]⁺.

### N-(5-cyclopentyl-1H-pyrazol-3-yl)-2-(6-methyl-2,6-diazaspiro[3.3]heptan-2-yl)pyrimidin-4-amine

To a solution of tert-butyl 6-[4-[(5-cyclopentyl-1H-pyrazol-3-yl)amino]pyrimidin-2-yl]-2,6-diazaspiro[3.3]heptane-2-carboxylate (153 mg, 0.359 mmol) in anhydrous THF (3 mL) under nitrogen was added a 2.4 M solution of lithium aluminum hydride ion THF (1.5 mL, 3.6 mmol). The reaction mixture was stirred at reflux for 1 hour. The reaction mixture was then cooled to 0° C., then quenched with water (0.15 mL), then a 10% w/v sodium hydroxide solution (0.15 mL), followed by water (0.5 mL). The reaction mixture was warmed to ambient temperature and stirred for 15 minutes, after which magnesium sulfate (ca. 1 g) was added, then the reaction mixture was stirred for a further 15 minutes. The reaction mixture was filtered, then the filter cake was washed with EtOAc (20 mL). The eluent was then concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure to afford the title compound as a solid (64 mg, 0.19 mmol, 53%).

¹H NMR (400 MHz, DMSO-d₆) δ 11.89 (s, 1H), 9.43 (s, 1H), 7.83 (d, J=5.9 Hz, 1H), 6.28 (s, 2H), 4.00 (s, 4H), 3.26 (s, 4H), 3.05-2.93 (m, 1H), 2.18 (s, 3H), 1.98 (ddd, J=11.2, 7.7, 4.8 Hz, 2H), 1.81-1.68 (m, 2H), 1.67-1.46 (m, 4H).

UPLC-MS (basic method, 4 min): Rt: 1.40 min, m/z: 340.2 [M+H]⁺.

### Example 44. Synthesis of Compound 51

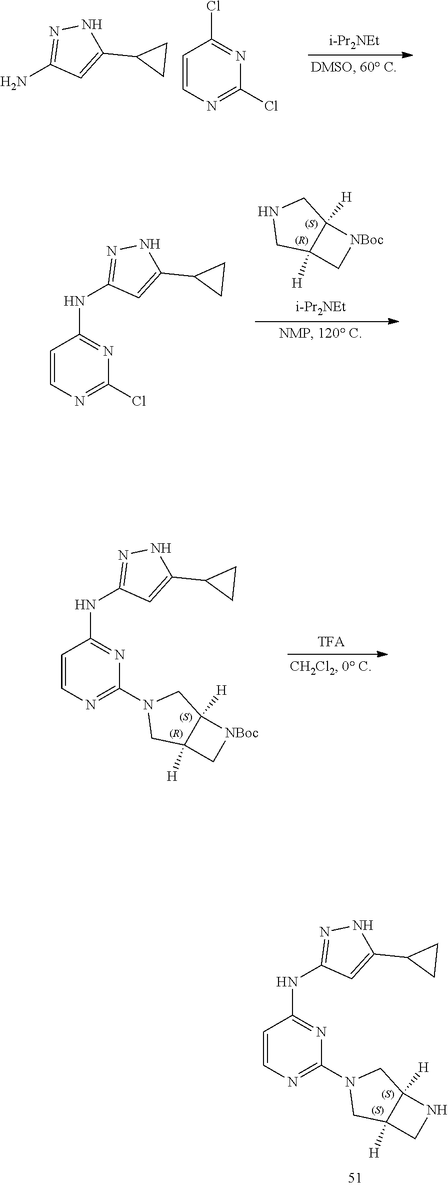

### tert-Butyl (1R,5S)-3-[4-[(5-Cyclopropyl-1H-pyrazol-3-yl)amino]pyrimidin-2-yl]-3,6-diazabicyclo[3.2.0]heptane-6-carboxylate

A solution of 2-chloro-N-(5-cyclopropyl-1H-pyrazol-3-yl)pyrimidin-4-amine (200 mg, 0.849 mmol), (1R,5S)-tert butyl 3,6-diazabicyclo[3.2.0]heptane-6-carboxylate (202 mg, 1.02 mmol) and N,N-diisopropylethylamine (0.37 mL, 2.12 mmol) in NMP (2 mL) was heated to 120° C. and stirred for 18 hours. The reaction mixture was cooled to ambient temperature, then added to stirring ice water (100 mL), resulting in the formation of a precipitate. The precipitate was collected by filtration, washed with water (2×10 mL) then dried under reduced pressure. The crude material was purified by automated flash column chromatography, over silica gel, eluting with a gradient of CH₂Cl₂: to 4:1 2M NH₃ in MeOH:CH₂Cl₂ to 80:20 CH₂Cl₂:2M NH₃ in MeOH to afford the title compound as a solid (156 mg, 0.393 mmol, 46%).

¹H NMR (400 MHz, DMSO-d₆) δ 11.89 (s, 1H), 9.42 (s, 1H), 7.83 (d, J=5.7 Hz, 1H), 6.21 (s, 2H), 4.69-4.61 (m, 1H), 4.13 (d, J=12.7 Hz, 1H), 4.00-3.83 (m, 2H), 3.42 (s, 1H), 3.16 (dd, J=11.8, 6.8 Hz, 1H), 3.11-2.99 (m, 2H), 1.81 (tt, J=8.4, 5.1 Hz, 1H), 1.33 (d, J=9.7 Hz, 10H), 0.86 (dd, J=8.6, 2.1 Hz, 2H), 0.67-0.56 (m, 2H).

UPLC-MS (basic method, 2 min): Rt: 1.04 min, m/z: 398.2 [M+H]⁺.

### N-(5-Cyclopropyl-1H-pyrazol-3-yl)-2-[(1S,5S)-3,6-diazabicyclo[3.2.0]heptan-3-yl]pyrimidin-4-amine

To a solution of tert-butyl (1R,5S)-3-[4-[(5-cyclopropyl-1H-pyrazol-3-yl)amino]pyrimidin-2-yl]-3,6-diazabicyclo[3.2.0]heptane-6-carboxylate (140 mg, 0.35 mmol) in CH₂Cl₂ (5 mL) was added trifluoroacetic acid (2 mL, 26 mmol) at ambient temperature and the reaction mixture was stirred for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure to afford the title compound as a solid (84 mg, 0.268 mmol, 80%).

¹H NMR (400 MHz, DMSO-d₆) δ 11.84 (s, 1H), 9.33 (s, 1H), 7.83 (d, J=5.7 Hz, 1H), 6.20 (s, 1H), 4.30 (dd, J=6.6, 4.7 Hz, 1H), 3.93-3.79 (m, 2H), 3.60 (t, J=7.7 Hz, 1H), 3.18-3.10 (m, 3H), 3.08-3.03 (m, 1H), 1.82 (tt, J=8.4, 5.0 Hz, 1H), 0.91-0.79 (m, 2H), 0.66-0.54 (m, 2H).

UPLC-MS (basic method, 4 min): Rt: 0.95 min, m/z: 298.1 [M+H]⁺.

### Example 45. Synthesis of Compound 64

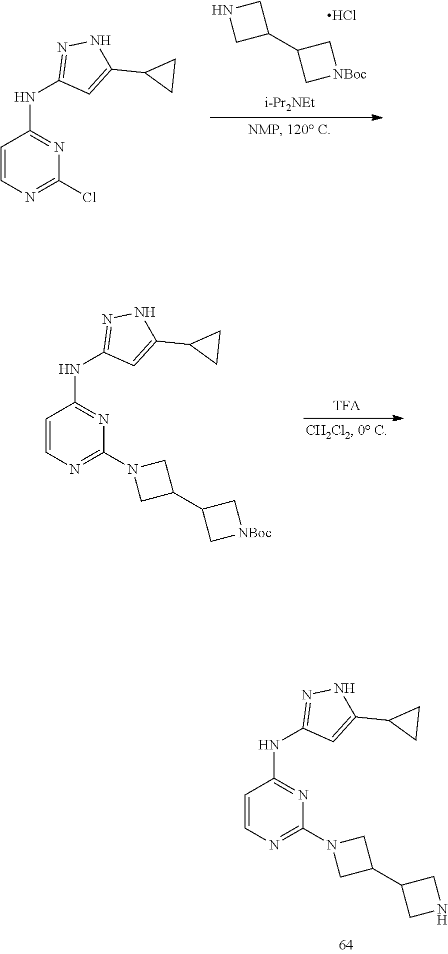 [SMILES: "i-Pr2NEt" ; Clc1nccc(Nc2cc(C3CC3)[nH]n2)n1 ; CC(C)(C)OC(=O)N1CC(C2CNC2)C1 ; Cl ; CC(C)(C)OC(=O)N1CC(C2CN(c3nccc(Nc4cc(C5CC5)[nH]n4)n3)C2)C1 ; c1cc(Nc2cc(C3CC3)[nH]n2)nc(N2CC(C3CNC3)C2)n1]

### tert-Butyl 3-[1-[4-[(5-Cyclopropyl-1H-pyrazol-3-yl)amino]pyrimidin-2-yl]azetidin-3-yl]azetidine-1-carboxylate

A solution of 2-chloro-N-(5-cyclopropyl-1H-pyrazol-3-yl)pyrimidin-4-amine (395 mg, 1.68 mmol), tert-butyl [3,3′-biazetidine]-1-carboxylate hydrochloride (1.20 eq, 500 mg, 2.01 mmol) and N,N-diisopropylethylamine (1.0 mL, 3.5 mmol) in NMP (6 mL) was heated to 120° C. and stirred for 20 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude residue was purified by automated flash column chromatography, eluting with a gradient of CH₂Cl₂ to 1:9 2 M NH₃ in CH₂Cl₂, to afford the title compound as a solid (205 mg, 0.478 mmol, 29%).

¹H NMR (400 MHz, DMSO-d₆) δ 11.90 (s, 1H), 9.43 (s, 1H), 7.84 (d, J=5.8 Hz, 1H), 6.48-6.08 (m, 2H), 4.12-4.02 (m, 2H), 3.95 (s, 2H), 3.62 (dd, J=8.6, 4.8 Hz, 2H), 3.53 (s, 2H), 2.99-2.75 (m, 2H), 1.89-1.72 (m, 1H), 1.46-1.29 (m, 9H), 0.98-0.83 (m, 2H), 0.75-0.56 (m, 2H).

UPLC-MS (basic method, 4 min): Rt: 1.56 min, m/z: 412.5 [M+H]⁺.

### 2-[3-(Azetidin-3-yl)azetidin-1-yl]-N-(5-cyclopropyl-1H-pyrazol-3-yl)pyrimidin-4-amine

To a solution of tert-butyl 3-[1-[4-[(5-cyclopropyl-1H-pyrazol-3-yl)amino]pyrimidin-2-yl]azetidin-3-yl]azetidine-1-carboxylate (97 mg, 0.24 mmol) in CH₂Cl₂ (4 mL) was added trifluoroacetic acid (4 mL, 52 mmol) at 0° C. The reaction mixture was warmed to ambient temperature and stirred for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC to afford the title compound as a solid (31 mg, 97 μmol, 41%).

¹H NMR (400 MHz, DMSO-d₆) δ 11.90 (s, 1H), 9.39 (s, 1H), 7.82 (d, J=5.7 Hz, 1H), 6.44-5.90 (m, 2H), 4.04 (t, J=8.0 Hz, 2H), 3.67 (dd, J=8.7, 4.5 Hz, 2H), 3.52 (t, J=7.2 Hz, 2H), 3.20 (t, 2H), 2.86 (s, 2H), 1.91-1.79 (m, 1H), 1.65 (s, 1H), 0.98-0.81 (m, 2H), 0.74-0.52 (m, 2H).

UPLC-MS (basic method, 4 min): Rt: 0.99 min, m/z: 312.2 [M+H]⁺.

### Example 46. Synthesis of Compound 14

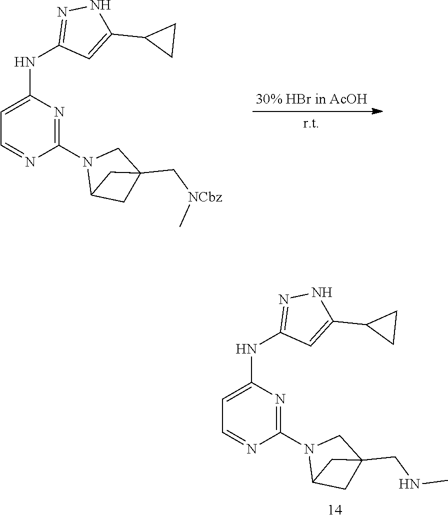 [SMILES: CN(CC12CC(C1)N(c1nccc(Nc3cc(C4CC4)[nH]n3)n1)C2)C(=O)OCc1ccccc1 ; CNCC12CC(C1)N(c1nccc(Nc3cc(C4CC4)[nH]n3)n1)C2]

### N-(5-cyclopropyl-1H-pyrazol-3-yl)-2-(4-((methylamino)methyl)-2-azabicyclo[2.1.1]hexan-2-yl)pyrimidin-4-amine

To a solution of benzyl ((2-(4-((5-cyclopropyl-1H-pyrazol-3-yl)amino)pyrimidin-2-yl)-2-azabicyclo[2.1.1]hexan-4-yl)methyl)(methyl)carbamate (200 mg, 0.435 mmol) in a 30% w/v solution of hydrogen bromide in acetic acid (3 mL) was stirred at ambient temperature for 3 hours. The reaction mixture was then concentrated to dryness under reduced pressure. The residue was purified by reversed-phase basic prepHPLC. The residue was then loaded onto an SCX cartridge, the cartridge was washed with methanol (10 mL), then the compound was eluted with 2 M NH₃ in MeOH (20 mL). The eluent was concentrated under reduced pressure to afford the title compound as a solid (77 mg, 0.23 mmol, 53%).

¹H NMR (400 MHz, methanol-d₄) δ 7.83 (d, J=5.9 Hz, 1H), 6.29-6.00 (m, 2H), 4.78 (s, 1H), 3.44 (s, 2H), 2.89 (s, 2H), 2.44 (s, 3H), 1.96-1.85 (m, 3H), 1.58-1.48 (m, 2H), 1.01-0.93 (m, 2H), 0.77-0.68 (m, 2H).

UPLC-MS (basic method, 2 min): Rt: 1.19 min, m/z=326.1 [M+H]⁺.

### Example 47. Synthesis of Compound 14

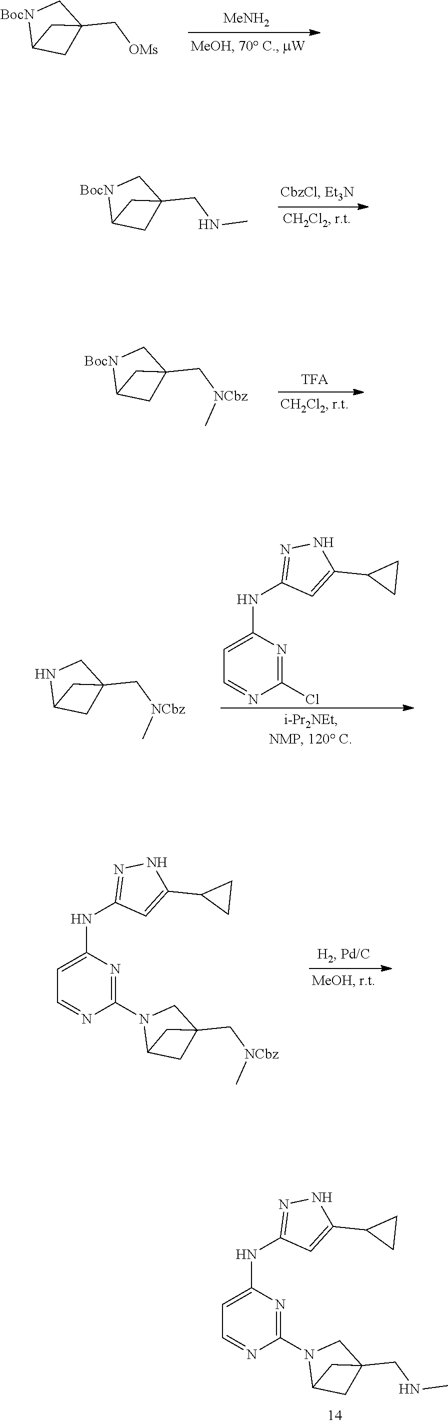

### tert-Butyl 4-(methylaminomethyl)-2-azabicyclo[2.1.1]hexane-2-carboxylate

2-Methyl-2-propanyl 4-{[(methylsulfonyl)oxy]methyl}-2-azabicyclo[2.1.1]hexane-2-carboxylate (1.018 g, 3.49 mmol) was dissolved in a 2.0 M solution of methylamine in methanol (60 mL, 120 mmol) and distributed across three 15-20 mL microwave vials. The reaction mixtures were successively heated to 70° C. under microwave irradiation for 2 hours, then the vials were recombined and concentrated to dryness. The crude residue was diluted with 0.5 M NaOH_{(aq)} (75 mL) and extracted with EtOAc (3×100 mL). The combined organic extracts were dried over Na₂SO₄, filtered and concentrated to dryness to afford the title compound as an oil (802 mg, 3.37 mmol, 96%).

¹H NMR (400 MHz, chloroform-d) δ 4.45-4.21 (m, 1H), 3.23 (s, 2H), 2.83 (s, 2H), 2.46 (s, 3H), 1.78-1.73 (m, 2H), 1.48-1.44 (m, 11H).

UPLC-MS (2 min, basic): rt=0.93 min, m/z=227.1 [M+H]⁺

### tert-Butyl 4-((((Benzyloxy)carbonyl)(methyl)amino)methyl)-2-azabicyclo[2.1.1]hexane-2-carboxylate

To a solution of tert-butyl 4-((methylamino)methyl)-2-azabicyclo[2.1.1]hexane-2-carboxylate (802 mg, 3.37 mmol) and triethylamine (0.74 mL, 5.31 mmol) in CH₂Cl₂ (45 mL) was added benzyl chloroformate (0.56 mL, 3.90 mmol) and the reaction mixture was stirred at ambient temperature for 3 hours. The reaction mixture was then diluted with water (50 mL) then extracted with CH₂Cl₂ (3×50 mL). The combined organic extracts were washed with a saturated aqueous brine solution (50 mL) then concentrated to dryness under reduced pressure. The crude material was purified by automated flash column chromatography, over silica gel, eluting with a gradient of 1:4 EtOAc:iso-hexane to 4:6 EtOAc:iso-hexane to afford the title compound as an oil (776 mg, 2.07 mmol, 58%).

¹H NMR (400 MHz, chloroform-d) δ 7.38-7.28 (m, 5H), 5.11 (d, J=9.1 Hz, 2H), 4.30 (d, J=13.0 Hz, 1H), 3.53 (d, J=16.2 Hz, 2H), 3.25-3.14 (d, 2H), 2.96 (d, J=6.1 Hz, 3H), 1.80-1.66 (m, 2H), 1.51-1.39 (m, 11H).

UPLC-MS (2 min, basic, ELSD): rt=1.26 min, m/z=361.2 [M+H]+

### Benzyl ((2-Azabicyclo[2.1.1]hexan-4-yl)methyl)(methyl)carbamate

To a solution of tert-butyl 4-((((benzyloxy)carbonyl)(methyl)amino)methyl)-2-azabicyclo[2.1.1]hexane-2-carboxylate (776 mg, 2.07 mmol) in CH₂Cl₂ (20 mL) was added trifluoroacetic acid (10.0 mL, 105 mmol) and the reaction mixture was stirred at room temperature for 30 minutes, then concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure to afford the title compound as an oil (487 mg, 1.78 mmol, 83%).

¹H NMR (400 MHz, chloroform-d) δ 7.38-7.14 (m, 5H), 5.03 (d, J=8.7 Hz, 2H), 3.59 (d, 1H), 3.47 (d, J=14.4 Hz, 2H), 2.88 (s, 3H), 2.73 (d, 2H), 1.55 (d, 2H), 1.35-1.16 (m, 2H).

UPLC-MS (basic method, 2 min): Rt: 0.89 min, m/z=261.1 [M+H]⁺.

### Benzyl ((2-(4-((5-Cyclopropyl-1H-pyrazol-3-yl)amino)pyrimidin-2-yl)-2-azabicyclo[2.1.1]hexan-4-yl)methyl)(methyl)carbamate

A solution of 2-chloro-N-(5-cyclopropyl-1H-pyrazol-3-yl)pyrimidin-4-amine (479 mg, 2.03 mmol), benzyl ((2-azabicyclo[2.1.1]hexan-4-yl)methyl)(methyl)carbamate (487 mg, 1.78 mmol) and N,N-diisopropylethylamine (0.77 mL, 4.44 mmol) in NMP (6 mL) was heated to 120° C. and stirred for 24 hours. The reaction mixture was cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (676 mg, 1.18 mmol, 66%).

¹H NMR (400 MHz, DMSO-d₆) δ δ 7.99 (d, J=5.7 Hz, 1H), 7.39-7.29 (m, 5H), 6.14 (s, 1H), 5.88 (s, 1H), 5.13 (s, 2H), 3.64-3.51 (m, 2H), 3.54-3.45 (m, 2H), 3.00 (d, J=6.6 Hz, 3H), 1.86 (ddd, J=13.5, 8.5, 5.1 Hz, 3H), 1.71-1.47 (m, 3H), 1.05-0.91 (m, 2H), 0.75 (dt, J=6.6, 4.8 Hz, 2H).

UPLC-MS (basic method, 2 min): Rt: 1.07 min, m/z=460.4 [M+H]⁺.

### N-(5-cyclopropyl-1H-pyrazol-3-yl)-2-(4-((methylamino)methyl)-2-azabicyclo[2.1.1]hexan-2-yl)pyrimidin-4-amine

To a solution of benzyl ((2-(4-((5-cyclopropyl-1H-pyrazol-3-yl)amino)pyrimidin-2-yl)-2-azabicyclo[2.1.1]hexan-4-yl)methyl)(methyl)carbamate (893 mg, 1.94 mmol) in methanol (25 mL) was added 10% w/w palladium on activated carbon (260 mg, 0.244 mmol) under a flow of nitrogen. The reaction was then exchanged for hydrogen and the reaction mixture was then stirred at room temperature for 6 hours. The atmosphere was exchanged for nitrogen and a suspension of 10% w/w palladium on activated carbon (282 mg, 0.265 mmol) in water (1 mL) was added under a flow of nitrogen. The atmosphere was exchanged for hydrogen and the reaction mixture was then stirred at room temperature overnight. The reaction mixture was then filtered through a pad of celite, and the filter cake was washed with EtOAc (10 mL). The eluent was concentrated to dryness, under reduced pressure. The residue was purified by reversed-phase basic prepHPLC to afford the title compound as a solid (206 mg, 0.613 mmol, 32%).

¹H NMR (400 MHz, methanol-d₄) δ 7.85-7.76 (m, 1H), 6.15 (s, 2H), 4.77 (q, J=1.8 Hz, 1H), 3.44 (s, 2H), 2.88 (d, J=3.7 Hz, 2H), 2.47-2.35 (m, 3H), 1.88 (dd, J=8.3, 5.1 Hz, 3H), 1.52 (dd, J=4.6, 1.9 Hz, 2H), 1.01-0.87 (m, 2H), 0.76-0.64 (m, 2H).

UPLC-MS (basic method, 2 min): Rt: 1.13 min, m/z=326.0 [M+H]⁺.

### Example 48. Synthesis of Compound 14

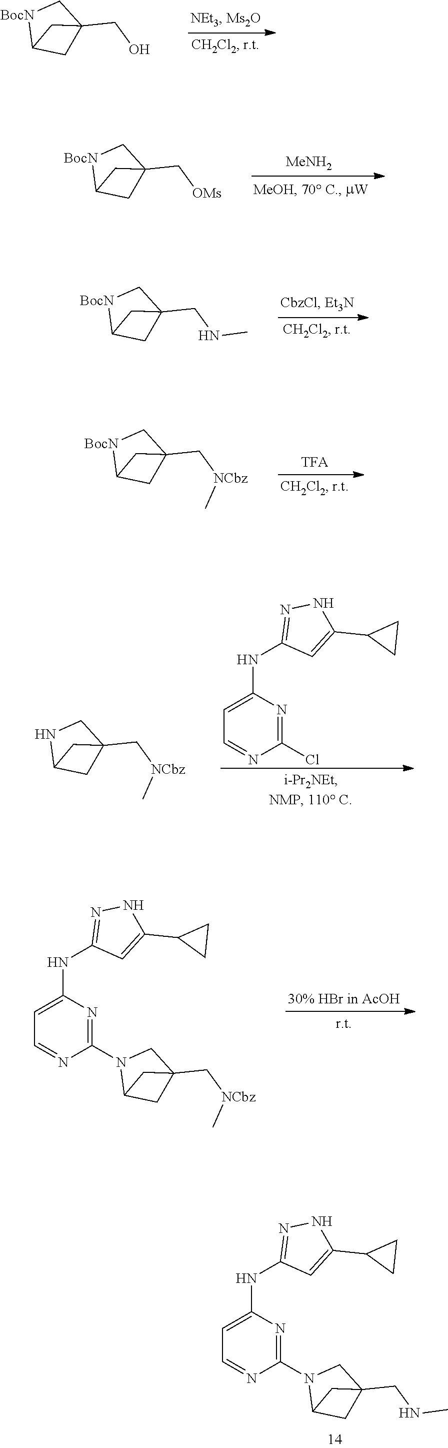

### tert-Butyl 4-(methylsulfonyloxymethyl)-2-azabicyclo[2.1.1]hexane-2-carboxylate

tert-Butyl 4-(hydroxymethyl)-2-azabicyclo[2.1.1]hexane-2-carboxylate (5.00 g, 23.4 mmol) was dissolved in CH₂Cl₂ (190 mL) and cooled to 0° C. Triethylamine (9.8 mL, 70.3 mmol) was added, followed by portion wise addition of methanesulfonic anhydride (8.17 g, 46.9 mmol) over 10 minutes then the reaction allowed to warm to room temperature and stir for 16 hours. The reaction mixture was quenched with water (1 L) and the organics extracted with CH₂Cl₂ (3×100 mL), and the combined organic extracts were dried over Na₂SO₄ and concentrated to dryness to afford the title compound as an oil (8.00 g, 24.7 mmol).

¹H NMR (400 MHz, chloroform-d) δ_{H} 4.40 (s, 2H), 3.29 (s, 2H), 3.03 (s, 3H), 1.87 (dt, J=4.3, 1.9 Hz, 2H), 1.54 (dd, J=4.6, 1.9 Hz, 2H), 1.46 (s, 9H).

### tert-Butyl 4-(methylaminomethyl)-2-azabicyclo[2.1.1]hexane-2-carboxylate

2-Methyl-2-propanyl 4-{[(methylsulfonyl)oxy]methyl}-2-azabicyclo[2.1.1]hexane-2-carboxylate (8.70 g, 29.9 mmol) was dissolved in a 2.0 M solution of methylamine in methanol (400 mL) in a 1 L Parr vessel, then the sealed reaction was heated at 70° C. for 16 hours. The vessel was allowed to cool to room temperature then the reaction mixture concentrated to dryness under reduced pressure to give the title compound as an oil (5.6 g, 23.5 mmol, 78% yield).

¹H NMR (400 MHz, chloroform-d) δ 4.32 (s, 1H), 3.21 (s, 2H), 2.81 (s, 2H), 2.44 (s, 3H), 1.73 (dt, J=4.1, 1.9 Hz, 2H), 1.44 (s, 9H), 1.50-1.38 (m, 2H).

### tert-Butyl 4-((((benzyloxy)carbonyl)(methyl)amino)methyl)-2-azabicyclo[2.1.1]hexane-2-carboxylate

To a solution of tert-butyl 4-((methylamino)methyl)-2-azabicyclo[2.1.1]hexane-2-carboxylate (5.6 g, 24.7 mmol) and triethylamine (5.17 mL, 37.12 mmol) in CH₂Cl₂ (56 mL) was added benzyl chloroformate (3.89 mL, 27.2 mmol) and the reaction mixture was stirred at ambient temperature for 1.5 hours. The reaction mixture was then diluted with water (200 mL) then extracted with CH₂Cl₂ (3×50 mL). The combined organic extracts were washed with a saturated aqueous brine solution (200 mL) then concentrated to dryness under reduced pressure. The crude material was purified by automated flash column chromatography, over silica gel, eluting with a gradient of 1:4 EtOAc:iso-hexane to 6:4 EtOAc:iso-hexane to afford the title compound as an oil (7.00 g, 16.51 mmol, 67%).

¹H NMR (400 MHz, chloroform-d) δ 7.38-7.27 (m, 5H), 5.10 (d, J=8.4 Hz, 2H), 4.31 (s, 1H), 3.51 (d, J=15.7 Hz, 2H), 3.23-3.13 (m, 2H), 2.95 (d, J=5.3 Hz, 2H), 1.67 (m, 2H), 1.45 (s, 11H).

UPLC-MS (basic method, 4 min, ELSD): Rt=2.03 min, m/z=261.2 [M−Boc+H]⁺.

### Benzyl ((2-azabicyclo[2.1.1]hexan-4-yl)methyl)(methyl)carbamate

To a solution of tert-butyl 4-((((benzyloxy)carbonyl)(methyl)amino)methyl)-2-azabicyclo[2.1.1]hexane-2-carboxylate (7.00 g, 19.42 mmol) in CH₂Cl₂ (120 mL) was added trifluoroacetic acid (30 mL, 400 mmol) and the reaction mixture was stirred at room temperature for 1 hour, then concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure to afford the title compound as an oil (3.94 g, 14.4 mmol, 74%).

¹H NMR (400 MHz, chloroform-d) δ 7.41-7.27 (m, 5H), 5.15-5.07 (m, 2H), 3.70-3.60 (m, 1H), 3.59-3.50 (m, 2H), 3.01-2.90 (m, 3H), 2.85-2.73 (m, 2H), 1.68-1.55 (m, 2H), 1.39-1.25 (m, 2H).

UPLC-MS (basic method, 2 min): Rt: 0.89 min, m/z=261.1 [M+H]⁺.

### Benzyl ((2-(4-((5-cyclopropyl-1H-pyrazol-3-yl)amino)pyrimidin-2-yl)-2-azabicyclo[2.1.1]hexan-4-yl)methyl)(methyl)carbamate

A solution of 2-chloro-N-(5-cyclopropyl-1H-pyrazol-3-yl)pyrimidin-4-amine (3.05 g, 12.94 mmol), benzyl ((2-azabicyclo[2.1.1]hexan-4-yl)methyl)(methyl)carbamate (3.94 g, 14.38 mmol) and N,N-diisopropylethylamine (6.26 mL, 35.95 mmol) in NMP (38 mL) was heated to 102° C. and stirred for 67 hours. Additional N,N-diisopropylethylamine (6.26 mL, 36.0 mmol) was added and the reaction heated at 110° C. for 20 hours. The reaction mixture was cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (676 mg, 1.18 mmol, 66%).

¹H NMR (400 MHz, DMSO-d₆) δ_{H} 11.87 (s, 1H), 9.31 (s, 1H), 7.79 (d, J=5.7 Hz, 1H), 7.36-7.22 (m, 5H), 6.34-5.99 (m, 2H), 5.04 (d, J=13.6 Hz, 2H), 4.63 (d, J=12.1 Hz, 1H), 3.55 (s, 2H), 2.89 (d, J=13.1 Hz, 3H), 1.79 (s, 2H), 1.72 (s, 1H), 1.39 (s, 1H), 1.34 (s, 1H), 0.86 (d, J=8.1 Hz, 2H), 0.62 (s, 2H).

UPLC-MS (basic method, 4 min): Rt: 1.74 min, m/z=460.4 [M+H]⁺.

### N-(5-Cyclopropyl-1H-pyrazol-3-yl)-2-(4-((methylamino)methyl)-2-azabicyclo[2.1.1]hexan-2-yl)pyrimidin-4-amine

To benzyl ((2-(4-((5-cyclopropyl-1H-pyrazol-3-yl)amino)pyrimidin-2-yl)-2-azabicyclo[2.1.1]hexan-4-yl)methyl)(methyl)carbamate (4.77 g, 10.38 mmol) was added a 30% w/v solution of hydrogen bromide in acetic acid (95 mL, 1.7 mol) and the reaction mixture was stirred at ambient temperature for 1 hour. The suspension was diluted with water (450 mL) and washed with CH₂Cl₂ (450 mL), and the aqueous layer added dropwise to a suspension of Na₂CO₃ (256 g) in water (730 mL). The solution was extracted with CH₂Cl₂ (3×500 mL) and the combined organics dried over Na₂SO₄ and concentrated to dryness under reduced pressure. The residue was purified by flash column chromatography, over silica gel, eluting with a gradient of 1:49 to 1:19 of 2 M NH₃ in MeOH:DCM to afford the title compound as a solid (1.84 g, 5.65 mmol, 54% yield).

¹H NMR (400 MHz, DMSO-d₆) δ_{H} 11.86 (s, 1H), 9.28 (s, 1H), 7.79 (d, J=5.7 Hz, 1H), 6.21 (s, 3H), 4.67-4.61 (m, 1H), 2.74 (s, 2H), 2.29 (s, 3H), 1.83 (ddd, J=13.5, 8.6, 5.0 Hz, 1H), 1.76 (m, 2H), 1.33 (m, 2H), 0.87 (m, 2H), 0.66-0.58 (m, 2H).

¹H NMR (400 MHz, methanol-d₄) δ 7.83 (d, J=6.0 Hz, 1H), 6.16 (s, 2H), 4.78 (m, 1H), 3.44 (s, 2H), 2.92 (s, 2H), 2.46 (s, 3H), 1.97-1.85 (m, 3H), 1.53 (m, 2H), 0.97 (m, 2H), 0.77-0.68 (m, 2H).

UPLC-MS (basic method, 2 min): Rt: 1.19 min, m/z=326.1 [M+H]⁺.

### Example 49. Synthesis of Compound 14

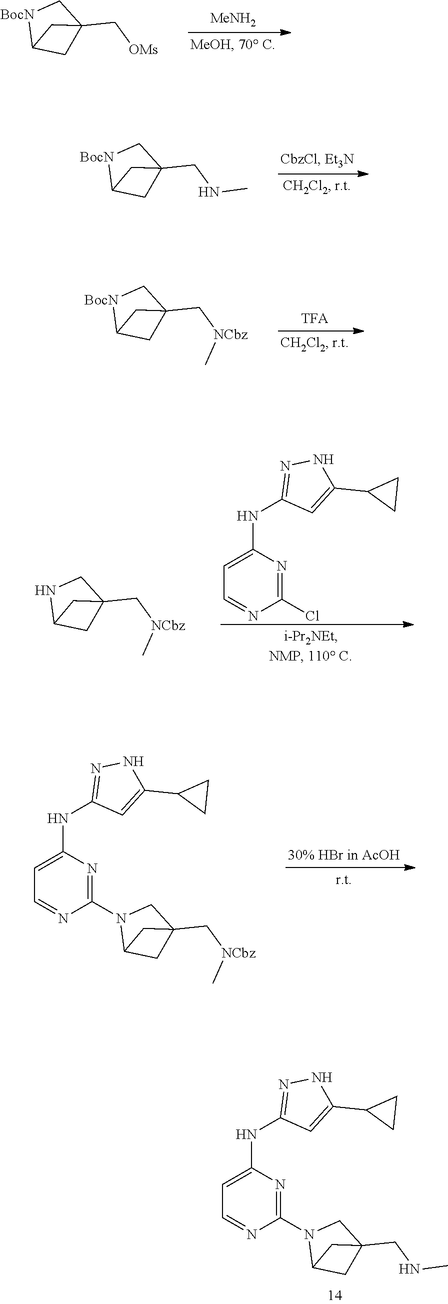

### tert-Butyl 4-(methylaminomethyl)-2-azabicyclo[2.1.1]hexane-2-carboxylate

2-Methyl-2-propanyl4-{[(methylsulfonyl)oxy]methyl}-2-azabicyclo[2.1.1]hexane-2-carboxylate (4.7 g, 16.1 mmol) was dissolved in a 2.0 M solution of methylamine in methanol (216 mL, 432 mmol) in a 300 mL Parr vessel, then the sealed reaction was heated at 70° C. for 16 hours. The vessel was allowed to cool to room temperature then the reaction mixture concentrated to dryness under reduced pressure then the residue taken up in brine (30 mL) and Na₂CO₃ (5.0 g) and extracted with EtOAc (3×30 mL). The combined organic extracts were dried over Na₂SO₄ and concentrated to dryness under reduced pressure to give the title compound as an oil (2.60 g, 10.9 mmol, 67% yield).

¹H NMR (400 MHz, chloroform-d) δ 4.32 (s, 1H), 3.22 (s, 2H), 2.82 (s, 2H), 2.45 (s, 3H), 1.79-1.69 (m, 2H), 1.45 (s, 11H).

### tert-Butyl 4-((((benzyloxy)carbonyl)(methyl)amino)methyl)-2-azabicyclo[2.1.1]hexane-2-carboxylate

To a solution of tert-butyl 4-((methylamino)methyl)-2-azabicyclo[2.1.1]hexane-2-carboxylate (3.6 g, 15.9 mmol) and triethylamine (3.3 mL, 23.9 mmol) in CH₂Cl₂ (32 mL) was added benzyl chloroformate (2.5 mL, 17.5 mmol) and the reaction mixture was stirred at ambient temperature for 3 hours. The reaction mixture was then diluted with water (100 mL) then extracted with CH₂Cl₂ (3×50 mL). The combined organic extracts were washed with a saturated aqueous brine solution (100 mL) then concentrated to dryness under reduced pressure. The crude material was purified by automated flash column chromatography, over silica gel, eluting with a gradient of 1:4 EtOAc:iso-hexane to 6:4 EtOAc:iso-hexane to afford the title compound as an oil (4.80 g, 12.5 mmol, 79%).

¹H NMR (400 MHz, chloroform-d) δ 7.40-7.27 (m, 5H), 5.11 (d, J=8.2 Hz, 2H), 4.30 (s, 1H), 3.53 (d, J=15.8 Hz, 2H), 3.31-3.08 (m, 2H), 2.99-2.93 (m, 3H), 1.80-1.67 (m, 2H), 1.55-1.36 (m, 11H).

UPLC-MS (basic method, 2 min): Rt=1.22 min, m/z=361.2 [M+H]⁺.

### Benzyl ((2-Azabicyclo[2.1.1]hexan-4-yl)methyl)(methyl)carbamate

To a solution of tert-butyl 4-((((benzyloxy)carbonyl)(methyl)amino)methyl)-2-azabicyclo[2.1.1]hexane-2-carboxylate (4.80 g, 13.3 mmol) in CH₂Cl₂ (80 mL) was added trifluoroacetic acid (21 mL, 273 mmol) and the reaction mixture was stirred at room temperature for 30 minutes, then concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure to afford the title compound as an oil (3.18 g, 12.2 mmol, 92%).

¹H NMR (400 MHz, chloroform-d) δ 7.41-7.27 (m, 5H), 5.15-5.07 (m, 2H), 3.70-3.60 (m, 1H), 3.59-3.50 (m, 2H), 3.01-2.90 (m, 3H), 2.85-2.73 (m, 2H), 1.68-1.55 (m, 2H), 1.39-1.25 (m, 2H).

UPLC-MS (basic method, 2 min): Rt: 0.80 min, m/z=261.1 [M+H]⁺.

### Benzyl ((2-(4-((5-Cyclopropyl-1H-pyrazol-3-yl)amino)pyrimidin-2-yl)-2-azabicyclo[2.1.1]hexan-4-yl)methyl)(methyl)carbamate

A solution of 2-chloro-N-(5-cyclopropyl-1H-pyrazol-3-yl)pyrimidin-4-amine (3.1 g, 13.3 mmol), benzyl ((2-azabicyclo[2.1.1]hexan-4-yl)methyl)(methyl)carbamate (3.2 g, 11.6 mmol) and N,N-diisopropylethylamine (5.1 mL, 29.0 mmol) in NMP (32 mL) was heated to 102° C. and stirred for 68 hours. Additional N,N-diisopropylethylamine (5.1 mL, 29.0 mmol) was added and the reaction heated at 110° C. for 20 hours. The reaction mixture was cooled to ambient temperature, then added dropwise to stirring ice water (600 mL), resulting in the formation of a precipitate. The precipitate was collected by filtration, then diluted with EtOAc (300 mL) and washed with saturated aqueous sodium hydrogen carbonate solution (2×125 mL). The organics were separated, dried over Na₂SO₄ then concentrated to dryness under reduced pressure. The crude material was purified by automated flash column chromatography, over silica gel, eluting with a gradient of EtOAc to ethanol/EtOAc/NH₄₀H (1:3:0.02 v/v) to afford the title compound as a foam (4.40 g, 9.10 mmol, 69%).

¹H NMR (400 MHz, chloroform-d, 50° C.) δ 8.00 (d, J=5.7 Hz, 1H), 7.40-7.27 (m, 6H), 6.75 (s, 1H), 6.13 (s, 1H), 5.89 (s, 1H), 5.15 (s, 2H), 4.81 (s, 1H), 3.61 (s, 2H), 3.53-3.40 (m, 2H), 3.01 (s, 3H), 1.91-1.78 (m, 3H), 1.46-1.35 (m, 2H), 1.01-0.89 (m, 2H), 0.78-0.71 (m, 2H).

UPLC-MS (basic method, 2 min): Rt: 1.07 min, m/z=460.2 [M+H]⁺.

### N-(5-cyclopropyl-1H-pyrazol-3-yl)-2-(4-((methylamino)methyl)-2-azabicyclo[2.1.1]hexan-2-yl)pyrimidin-4-amine

To benzyl ((2-(4-((5-cyclopropyl-1H-pyrazol-3-yl)amino)pyrimidin-2-yl)-2-azabicyclo[2.1.1]hexan-4-yl)methyl)(methyl)carbamate (1.30 g, 2.83 mmol) was added a 30% w/v solution of hydrogen bromide in acetic acid (1.6 mL, 26.0 mmol), and the reaction mixture was stirred at ambient temperature for 80 minutes. The suspension was diluted with water (120 mL) and washed with CH₂Cl₂ (120 mL), and the aqueous layer added dropwise to a suspension of Na₂CO₃ (70 g) in water (200 mL). The solution was extracted with CH₂Cl₂ (3×150 mL) and the combined organics dried over Na₂SO₄ and concentrated to dryness under reduced pressure. The crude compound was purified by reversed-phase basic prepHPLC to afford the title compound as a solid (509 g, 1.55 mmol, 55% yield).

¹H NMR (400 MHz, DMSO-d₆) δ 11.90 (s, 1H), 9.34 (s, 1H), 7.83 (d, J=5.7 Hz, 1H), 6.42-5.94 (m, 2H), 4.68 (s, 1H), 2.75 (s, 2H), 2.31 (s, 3H), 1.86 (tt, J=8.4, 5.1 Hz, 1H), 1.82-1.75 (m, 2H), 1.40-1.32 (m, 2H), 0.94-0.87 (m, 2H), 0.69-0.63 (m, 2H).

¹H NMR (400 MHz, methanol-d₄) δ 7.83 (d, J=6.0 Hz, 1H), 6.32-5.96 (m, 2H), 4.78 (s, 1H), 3.44 (s, 2H), 2.89 (s, 2H), 2.44 (s, 3H), 1.98-1.85 (m, 3H), 1.56-1.48 (m, 2H), 1.01-0.93 (m, 2H), 0.75-0.69 (m, 2H).

UPLC-MS (basic method, 4 min): Rt: 1.13 min, m/z=326.1 [M+H]⁺.

### Example 50: Synthesis of Compound 14

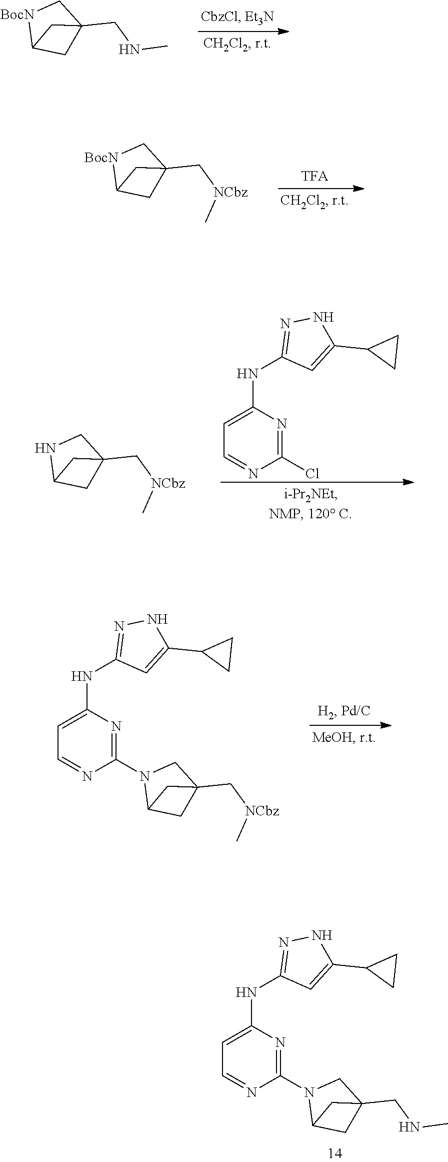 [SMILES: CNCC12CC(C1)N(C(=O)OC(C)(C)C)C2 ; CN(CC12CC(C1)N(C(=O)OC(C)(C)C)C2)C(=O)OCc1ccccc1 ; CN(CC12CNC(C1)C2)C(=O)OCc1ccccc1 ; Clc1nccc(Nc2cc(C3CC3)[nH]n2)n1 ; CN(CC12CC(C1)N(c1nccc(Nc3cc(C4CC4)[nH]n3)n1)C2)C(=O)OCc1ccccc1 ; CNCC12CC(C1)N(c1nccc(Nc3cc(C4CC4)[nH]n3)n1)C2]

### tert-Butyl 4-((((Benzyloxy)carbonyl)(methyl)amino)methyl)-2-azabicyclo[2.1.1]hexane-2-carboxylate

To a solution of tert-butyl 4-((methylamino)methyl)-2-azabicyclo[2.1.1]hexane-2-carboxylate (815 mg, 3.60 mmol) and triethylamine (753 μL, 5.40 mmol) in CH₂Cl₂ (10 mL) was added benzyl chloroformate (565 μL, 3.96 mmol) and the reaction mixture was stirred at ambient temperature for 3 hours. The reaction mixture was then diluted with water (50 mL) then extracted with CH₂Cl₂ (3×50 mL). The combined organic extracts were washed with a saturated aqueous brine solution (50 mL) then concentrated to dryness under reduced pressure. The crude material was purified by automated flash column chromatography, over silica gel, eluting with a gradient of 1:4 EtOAc:iso-hexane to 4:6 EtOAc:iso-hexane to afford the title compound as an oil (1.16 g, 3.12 mmol, 87%).

¹H NMR (400 MHz, chloroform-d) δ 7.39-7.28 (m, 5H), 5.14-5.08 (m, 2H), 4.29 (s, 1H), 3.57-3.49 (m, 2H), 3.29-3.11 (m, 2H), 2.99-2.93 (m, 3H), 1.78-1.66 (m, 2H), 1.46 (s, 11H).

UPLC-MS (basic method, 2 min): Rt: 1.26 min, m/z=361.1 [M+H]⁺.

### Benzyl ((2-azabicyclo[2.1.1]hexan-4-yl)methyl)(methyl)carbamate

To a solution of tert-butyl 4-((((benzyloxy)carbonyl)(methyl)amino)methyl)-2-azabicyclo[2.1.1]hexane-2-carboxylate (1.15 g, 3.19 mmol) in CH₂Cl₂ (10 mL) was added trifluoroacetic acid (5.0 mL, 65 mmol) and the reaction mixture was stirred at room temperature for 30 minutes. The crude residue was loaded onto an SCX cartridge, the cartridge was then washed with methanol, then the compound was eluted with 2 M NH₃ in methanol, and the eluent was concentrated to dryness under reduced pressure to afford the title compound as an oil (760 mg, 2.92 mmol, 92%).

¹H NMR (400 MHz, chloroform-d) δ 7.39-7.29 (m, 5H), 5.15-5.08 (m, 2H), 3.60-3.51 (m, 2H), 2.99-2.93 (m, 3H), 2.84-2.72 (m, 2H), 1.65-1.55 (m, 2H), 1.36-1.23 (m, 2H).

UPLC-MS (basic method, 2 min): Rt: 0.86 min, m/z=261.1 [M+H]⁺.

### Benzyl ((2-(4-((5-Cyclopropyl-1H-pyrazol-3-yl)amino)pyrimidin-2-yl)-2-azabicyclo[2.1.1]hexan-4-yl)methyl)(methyl)carbamate

A solution of 2-chloro-N-(5-cyclopropyl-1H-pyrazol-3-yl)pyrimidin-4-amine (570 mg, 2.42 mmol), benzyl ((2-azabicyclo[2.1.1]hexan-4-yl)methyl)(methyl)carbamate (756 mg, 2.90 mmol) and N,N-diisopropylethylamine (0.93 mL, 5.3 mmol) in NMP (6 mL) was heated to 120° C. in a sealed tube and stirred for 18 hours. The reaction mixture was cooled to ambient temperature, then added dropwise to stirring ice water (200 mL), resulting in the formation of a precipitate. The precipitate was collected by filtration, washed with water (2×10 mL) then dried under reduced pressure to afford the title compound as a solid (970 mg, 2.11 mmol, 87%).

¹H NMR (400 MHz, DMSO-d₆) δ 11.90 (s, 1H), 9.34 (s, 1H), 7.84 (d, J=5.6 Hz, 1H), 7.45-7.25 (m, 5H), 6.26 (s, 1H), 6.16 (s, 1H), 5.12-5.04 (m, 2H), 4.71-4.63 (m, 1H), 3.59 (s, 2H), 2.97-2.89 (m, 3H), 1.96-1.72 (m, 3H), 1.45-1.36 (m, 2H), 0.98-0.86 (m, 2H), 0.71-0.64 (m, 2H).

UPLC-MS (basic method, 2 min): Rt: 1.07 min, m/z=460.4 [M+H]⁺.

### N-(5-cyclopropyl-1H-pyrazol-3-yl)-2-(4-((methylamino)methyl)-2-azabicyclo[2.1.1]hexan-2-yl)pyrimidin-4-amine

To a solution of benzyl ((2-(4-((5-cyclopropyl-1H-pyrazol-3-yl)amino)pyrimidin-2-yl)-2-azabicyclo[2.1.1]hexan-4-yl)methyl)(methyl)carbamate (220 mg, 0.479 mmol) in Methanol (5 mL) was added a suspension of 10% w/w palladium on activated carbon (51 mg, 48 mol) in water (0.1 mL) under a flow of nitrogen. The reaction vessel was then placed under vacuum and backfilled with nitrogen three times. The reaction vessel was then placed under vacuum, then back-filled with hydrogen a total of three times. The reaction mixture was then stirred at room temperature for 3 hours. The reaction mixture was then filtered through a pad of celite, and the filter cake was washed with EtOAc (10 mL). The eluent was concentrated to dryness, under reduced pressure. The residue was purified by reversed-phase basic prepHIPLC to afford the title compound as a solid (90 mg, 0.277 mmol, 58%).

¹H NMR (400 MHz, DMSO-d₆) δ 11.89 (s, 1H), 9.31 (s, 1H), 7.83 (d, J=5.7 Hz, 1H), 6.35-5.97 (m, 2H), 4.68 (s, 1H), 3.32 (s, 2H), 2.75 (s, 2H), 2.32 (s, 3H), 1.86 (tt, J=8.5, 5.1 Hz, 1H), 1.82-1.76 (m, 2H), 1.42-1.31 (m, 2H), 0.95-0.86 (m, 2H), 0.69-0.62 (m, 2H).

¹H NMR (400 MHz, methanol-d₄) δ 7.83 (d, J=6.0 Hz, 1H), 6.27-5.92 (m, 1H), 4.78 (s, 1H), 3.44 (s, 2H), 2.89 (s, 2H), 2.44 (s, 3H), 1.97-1.84 (m, 3H), 1.58-1.47 (m, 1H), 1.02-0.92 (m, 2H), 0.77-0.68 (m, 2H).

UPLC-MS (basic method, 2 min): Rt: 1.15 min, m/z=326.0 [M+H]⁺.

### Example 51: Synthesis of Compound 30

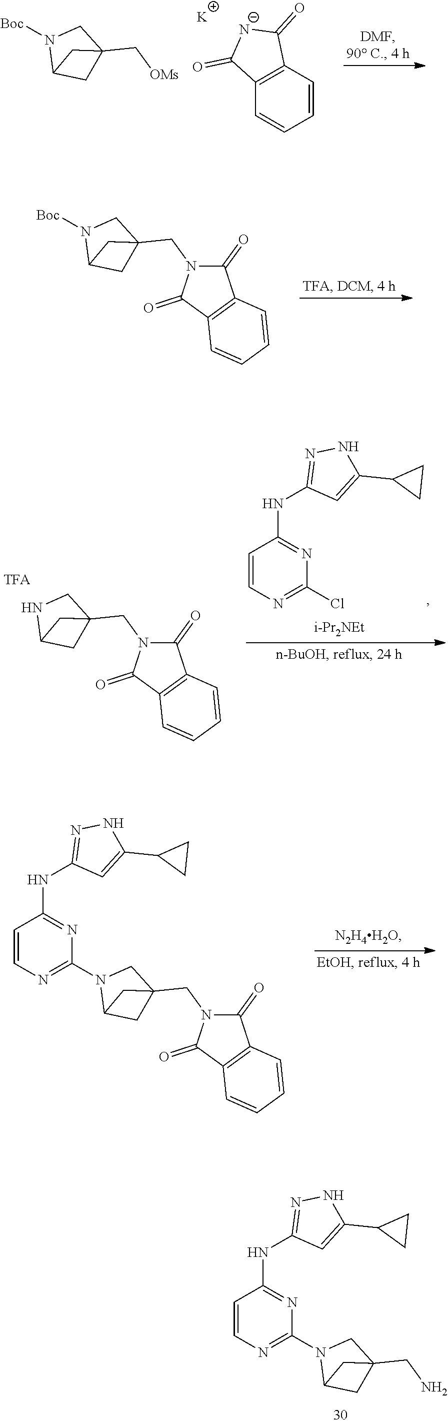

### tert-Butyl 4-[(1,3-Dioxoisoindolin-2-yl)methyl]-2-azabicyclo[2.1.1]hexane-2-carboxylate

2-methyl-2-propanyl4-{[(methylsulfonyl)oxy]methyl}-2-azabicyclo[2.1.1]hexane-2-carboxylate (600 mg, 2.06 mmol) and potassium phthalimide (458 mg, 2.47 mmol) were dissolved in DMF (6 mL) and reaction mixture was heated at 90° C. under a nitrogen atmosphere for 4 h. Reaction mixture was poured in ice-water and precipitated solid was collected by filtration. The filter cake was washed with iso-hexane (20 mL) and dried under vacuum to afford the title compound as a solid (590 mg, 1.72 mmol, 84%).

¹H NMR (400 MHz, DMSO-d₆) δ 7.90-7.74 (m, 4H), 4.07 (t, J=1.8 Hz, 1H), 3.82 (s, 2H), 3.08 (s, 2H), 1.71 (dt, J=4.2, 1.8 Hz, 2H), 1.33 (s, 9H), 1.31-1.27 (m, 2H).

UPLC-MS analysis: (2 min, basic): rt=1.18 min, characteristic m/z not observed.

### 2-(2-azabicyclo[2.1.1]hexan-4-ylmethyl)isoindoline-1,3-dione;2,2,2-trifluoroacetic acid

tert-butyl 4-[(1,3-dioxoisoindolin-2-yl)methyl]-2-azabicyclo[2.1.1]hexane-2-carboxylate (520 mg, 1.52 mmol) was dissolved in DCM (10 mL) and reaction mixture was cooled to 0° C. Trifluoroacetic acid (1.2 mL, 15 mmol) was added to it and reaction mixture was stirred for 4 h. The solvent was evaporated to dryness under reduced pressure to afford the title compound as a solid (505 mg, 1.42 mmol, 93%).

¹H NMR (400 MHz, DMSO-d₆) δ 8.86 (s, 2H), 7.89-7.76 (m, 4H), 4.02 (d, J=1.8 Hz, 1H), 3.87 (s, 2H), 3.18-3.07 (m, 2H), 1.88 (d, J=5.8 Hz, 2H), 1.52-1.37 (m, 2H).

UPLC-MS analysis: (2 min, basic): rt=0.80 min, m/z=243.1 [M+H]⁺.

### 2-[[2-[4-[(5-Cyclopropyl-1H-pyrazol-3-yl)amino]pyrimidin-2-yl]-2-azabicyclo[2.1.1]hexan-4-yl]methyl]isoindoline-1,3-dione

2-(2-azabicyclo[2.1.1]hexan-4-ylmethyl)isoindoline-1,3-dione;2,2,2-trifluoroacetic acid (505 mg, 1.42 mmol), 2-chloro-N-(5-cyclopropyl-1H-pyrazol-3-yl)pyrimidin-4-amine (334 mg, 1.42 mmol), and N,N-diisopropylethylamine (0.74 mL, 4.25 mmol) were dissolved in n-butanol (6 mL) and the reaction mixture was heated at reflux for 24 h. Reaction mixture was evaporated and ice-water was added to it. Precipitated brown solid was filtered off and washed with 10% ethyl acetate/isohexane (2×10 mL). The precipitate was dried under vacuum to obtain the title compound as a solid (460 mg, 1.04 mmol, 74%).

¹H NMR (400 MHz, DMSO-d₆) δ 12.17 (s, 1H), 7.93-7.73 (m, 5H), 6.29 (s, 2H), 4.72 (d, J=1.7 Hz, 1H), 3.97 (s, 2H), 3.43 (s, 2H), 1.95 (s, 2H), 1.92-1.83 (m, 1H), 1.54 (d, J=4.6 Hz, 2H), 1.08-0.85 (m, 2H), 0.80-0.57 (m, 2H).

UPLC-MS analysis: (2 min, basic): rt=1.03 min, m/z=442.3 [M+H]⁺.

### 2-[4-(aminomethyl)-2-azabicyclo[2.1.1]hexan-2-yl]-N-(5-cyclopropyl-1H-pyrazol-3-yl)pyrimidin-4-amine

2-[[2-[4-[(5-cyclopropyl-1H-pyrazol-3-yl) amino]pyrimidin-2-yl]-2-azabicyclo[2.1.1]hexan-4-yl]methyl]isoindoline-1,3-dione (460 mg, 1.04 mmol) was dissolved in ethanol (14 mL) and hydrazine hydrate (0.20 mL, 4.2 mmol) was added. The reaction mixture was heated to reflux for a period of 4 h. The reaction mixture was cooled and ethanol (20 mL) was added to it. The precipitated solid was removed by filtration, then the filtrate was concentrated to dryness under reduced pressure. The residue was purified by reversed-phase preparative HPLC, under basic conditions. The residue was then dissolved in MeOH, the loaded onto an SCX-2 cartridge. The cartridge was washed with MeOH, then the compound was eluted with 2 M NH₃ in MeOH. The eluent was concentrated to dryness under reduced pressure to afford the title compound as a solid. (107 mg, 0.344 mmol, 33%).

¹H NMR (400 MHz, Methanol-d₄) δ 7.84 (d, J=5.9 Hz, 1H), 6.16 (s, 2H), 4.79 (t, J=1.8 Hz, 1H), 3.42 (s, 2H), 3.00 (s, 2H), 1.95-1.85 (m, 3H), 1.50 (dd, J=4.5, 1.9 Hz, 2H), 0.97 (d, J=8.2 Hz, 2H), 0.76-0.68 (m, 2H).

UPLC-MS analysis: (4 min, basic): rt=1.12 min, m/z=312.1 [M+H]+.

### 2-[4-(aminomethyl)-2-azabicyclo[2.1.1]hexan-2-yl]-N-(5-cyclopropyl-1H-pyrazol-3-yl)pyrimidin-4-amine

To a solution of 2-[[2-[4-[(5-cyclopropyl-1H-pyrazol-3-yl)amino]pyrimidin-2-yl]-2-azabicyclo[2.1.1]hexan-4-yl]methyl]isoindoline-1,3-dione (120 mg, 0.271 mmol) in EtOH (3 mL) was added hydrazine hydrate (40 μL, 0.41 mmol) and the reaction mixture was heated to 60° C. for 3 hours. The reaction mixture was cooled to room temperature, then filtered through a pad of celite. The filtrate was then concentrated to dryness under reduced pressure. The crude compound was loaded onto an SCX-2 cartridge, the cartridge was then washed with methanol, then the compound was eluted with 2 M NH₃ in methanol, and the eluent was concentrated to dryness under reduced pressure. Sample was further purified by reversed-phase basic prepHPLC to afford the title compound as a solid (21 mg, 69 μmol, 25%).

¹H NMR (400 MHz, DMSO-d₆) δ 11.89 (s, 1H), 9.29 (s, 1H), 7.83 (d, J=5.7 Hz, 1H), 4.71-4.66 (m, 1H), 2.85 (s, 2H), 1.92-1.81 (m, 1H), 1.80-1.75 (m, 2H), 1.36-1.30 (m, 2H), 0.94-0.87 (m, 2H), 0.70-0.62 (m, 2H).

¹H NMR (400 MHz, methanol-d₄) δ 7.84 (d, J=5.9 Hz, 1H), 6.16 (s, 2H), 4.82-4.77 (m, 1H), 3.42 (s, 2H), 3.06-3.01 (m, 2H), 1.95-1.92 (m, 2H), 1.91-1.87 (m, 1H), 1.56-1.48 (m, 2H), 1.00-0.94 (m, 2H), 0.76-0.68 (m, 2H).

UPLC-MS (basic method, 4 min): Rt: 1.07 min, m/z=312.1 [M+H]⁺.

### Example 52: Synthesis of Compound 34

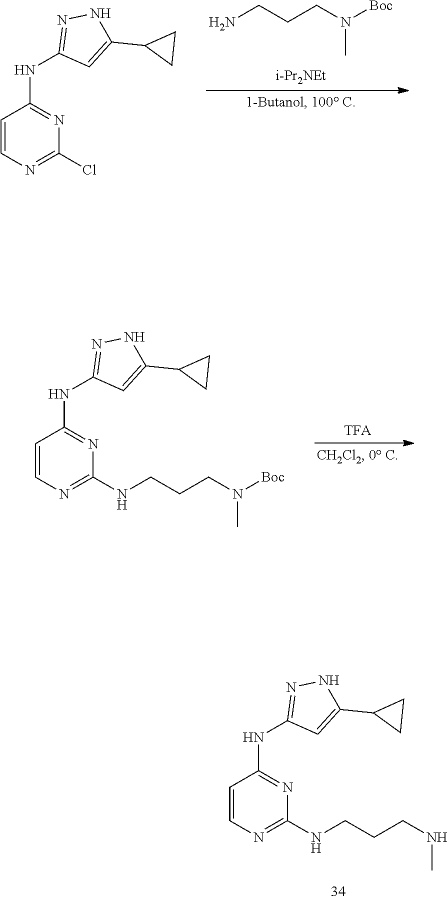 [SMILES: "i-Pr2NEt" ; Clc1nccc(Nc2cc(C3CC3)[nH]n2)n1 ; CN(CCCN)C(=O)OC(C)(C)C ; CN(CCCNc1nccc(Nc2cc(C3CC3)[nH]n2)n1)C(=O)OC(C)(C)C ; CNCCCNc1nccc(Nc2cc(C3CC3)[nH]n2)n1]

### tert-Butyl N-[3-[[4-[(5-cyclopropyl-1H-pyrazol-3-yl)amino]pyrimidin-2-yl]amino]propyl]-N-methyl-carbamate

A solution of 2-chloro-N-(5-cyclopropyl-1H-pyrazol-3-yl)pyrimidin-4-amine (300 mg, 1.27 mmol), N-methylpropane-1,3-diamine, N-Boc protected (0.61 mL, 3.2 mmol) and N,N-diisopropylethylamine (0.78 mL, 4.5 mmol) in 1-butanol (7 mL) was heated to 100° C. and stirred for 96 hours. The reaction mixture was then cooled to ambient temperature and the solvent removed under reduced pressure. The residue was dissolved in CH₂Cl₂ (20 mL) and washed with water (2×20 mL) and the organics were dried (Na₂SO₄) and concentrated to dryness under reduced pressure. The residue was purified by normal-phase column chromatography over silica gel, eluting with a gradient of CH₂Cl₂ to 9:1 CH₂Cl₂:0.1% NEt₃ in MeOH to afford the title compound as a solid (199 mg, 0.514 mmol, 40%).

¹H NMR (400 MHz, chloroform-d) δ 7.96 (d, J=5.8 Hz, 1H), 6.22 (s, 1H), 5.87 (s, 1H), 3.47-3.38 (m, 3H), 2.87 (s, 3H), 1.92-1.81 (m, 3H), 1.51-1.44 (m, 11H), 1.00-0.91 (m, 2H), 0.79-0.75 (m, 2H).

UPLC-MS (basic method, 2 min): Rt: 1.00 min, m/z=388.2 [M+H]⁺.

### N⁴-(5-cyclopropyl-1H-pyrazol-3-yl)-N²-[3-(methylamino)propyl]pyrimidine-2,4-diamine

To a solution of tert-butyl N-[3-[[4-[(5-cyclopropyl-1H-pyrazol-3-yl)amino]pyrimidin-2-yl]amino]propyl]-N-methyl-carbamate (196 mg, 0.506 mmol) in CH₂Cl₂ (4 mL) was added trifluoroacetic acid (1.0 mL, 26 mmol) at 0° C. and the reaction mixture allowed to warm to room temperature and stir for 1 hour. The reaction mixture was concentrated under reduced pressure and the crude compound was loaded onto an SCX-2 cartridge. The cartridge was then washed with methanol, then the compound was eluted with 2 M NH₃ in methanol, and the eluent was concentrated to dryness under reduced pressure to afford the title compound as a solid (114 mg, 0.397 mmol, 78%).

¹H NMR (400 MHz, DMSO-d₆) δ 9.36 (s, 1H), 7.77 (d, J=5.6 Hz, 1H), 6.71 (s, 1H), 6.11 (s, 2H), 3.30-3.22 (m, 2H), 2.53-2.51 (m, 2H), 2.26 (s, 3H), 1.89-1.78 (m, 1H), 1.70-1.58 (m, 2H), 0.94-0.84 (m, 2H), 0.70-0.60 (m, 2H).

¹H NMR (400 MHz, methanol-d₄) δ 7.79 (d, J=5.9 Hz, 1H), 6.10 (d, J=5.9 Hz, 1H), 5.99 (s, 1H), 3.41 (t, J=6.7 Hz, 2H), 2.67 (t, J=7.1 Hz, 2H), 2.38 (s, 3H), 1.93-1.86 (m, 1H), 1.86-1.77 (m, 2H), 1.00-0.91 (m, 2H), 0.76-0.67 (m, 2H).

UPLC-MS (basic method, 4 min): Rt: 0.97 min, m/z=288.1 [M+H]⁺.

### Example 53: Synthesis of Compound 36

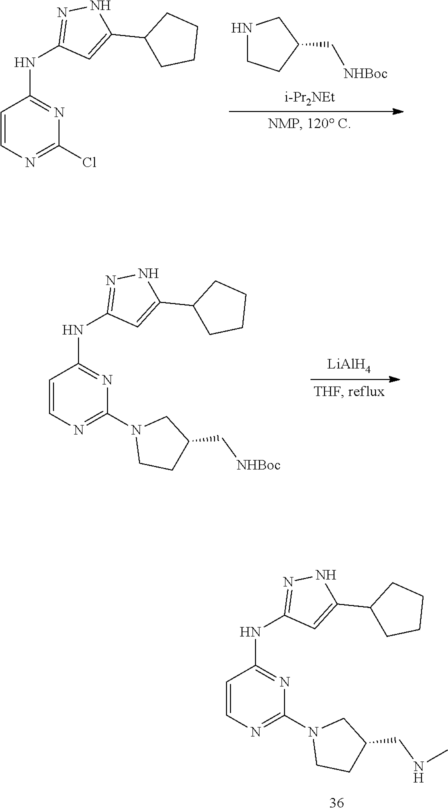 [SMILES: "i-Pr2NEt" ; Clc1nccc(Nc2cc(C3CCCC3)[nH]n2)n1 ; CC(C)(C)OC(=O)NC[C@@H]1CCNC1 ; CC(C)(C)OC(=O)NC[C@@H]1CCN(c2nccc(Nc3cc(C4CCCC4)[nH]n3)n2)C1 ; "LiAlH4" ; CNC[C@@H]1CCN(c2nccc(Nc3cc(C4CCCC4)[nH]n3)n2)C1]

### tert-Butyl N-[[(3S)-1-[4-[(5-Cyclopentyl-1H-pyrazol-3-yl)amino]pyrimidin-2-yl]pyrrolidin-3-yl]methyl]carbamate

A solution of 2-chloro-N-(5-cyclopentyl-1H-pyrazol-3-yl)pyrimidin-4-amine (442 mg, 1.68 mmol), tert-butyl N-[(3R)-pyrrolidin-3-ylmethyl]carbamate hydrochloride (269 mg, 1.14 mmol), and N,N-diisopropylethylamine (1.0 mL, 5.9 mmol) in NMP (4 mL) was heated to 120° C. and stirred for 20 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude was purified was by flash column chromatography, eluting with 9:1 CH₂Cl₂:2 M NH₃ in MeOH, to afford the title compound as an oil (315 mg, 0.597 mmol, 79%).

¹H NMR (400 MHz, DMSO-d₆) δ 11.87 (s, 1H), 9.36 (s, 1H), 7.82 (d, J=5.8 Hz, 1H), 6.98 (s, 2H), 6.20 (s, 1H), 3.55 (s, 1H), 2.98 (s, 3H), 2.37 (s, 1H), 2.18 (t, J=8.1 Hz, 1H), 2.05-1.82 (m, 4H), 1.75-1.53 (m, 8H), 1.38 (s, 9H).

UPLC-MS (basic method, 2 min): Rt: 1.10 min, m/z: 428.4 [M+H]⁺.

### N-(5-cyclopentyl-1H-pyrazol-3-yl)-2-[(3S)-3-(methylaminomethyl)pyrrolidin-1-yl]pyrimidin-4-amine

To a solution of tert-butyl N-[[(3S)-1-[4-[(5-cyclopentyl-1H-pyrazol-3-yl)amino]pyrimidin-2-yl]pyrrolidin-3-yl]methyl]carbamate (115 mg, 0.269 mmol) in anhydrous THE (10 mL) was added a 2.4 M solution of lithium aluminum hydride in THE (1.3 mL, 2.7 mmol) dropwise at 0° C. The reaction mixture was heated to reflux and stirred for 3 hours. The reaction mixture was then cooled to 0° C., then quenched with water (1.2 mL), then a 15% w/v sodium hydroxide solution (1.2 mL), followed by water (3.6 mL). The reaction mixture was warmed to ambient temperature and stirred for 10 minutes, after which magnesium sulfate (ca. 1 g) was added and the reaction mixture was stirred for a further 10 minutes. The reaction mixture was passed through a pad of celite, and the filter cake washed with EtOAc (20 mL). The filtrate was then concentrated to dryness under reduced pressure. The residue was purified by reversed-phase basic prepHPLC to afford the title compound as a solid (6.4 mg, 18.2 μmol, 6.78%).

¹H NMR (400 MHz, DMSO-d₆) δ 11.85 (s, 1H), 9.31 (s, 1H), 7.82 (d, J=5.7 Hz, 1H), 6.41 (s, 1H), 6.18 (s, 1H), 3.64 (t, J=9.4 Hz, 1H), 3.59-3.54 (m, 1H), 3.44-3.35 (m, 1H), 3.22-3.11 (m, 1H), 3.03-2.96 (m, 1H), 2.41-2.32 (m, 1H), 2.29 (s, 3H), 2.08-1.90 (m, 4H), 1.76-1.53 (m, 8H).

UPLC-MS (basic method, 4 min): Rt: 1.40 min, m/z: 342.2 [M+H]⁺.

### Example 54: Synthesis of Compound 39

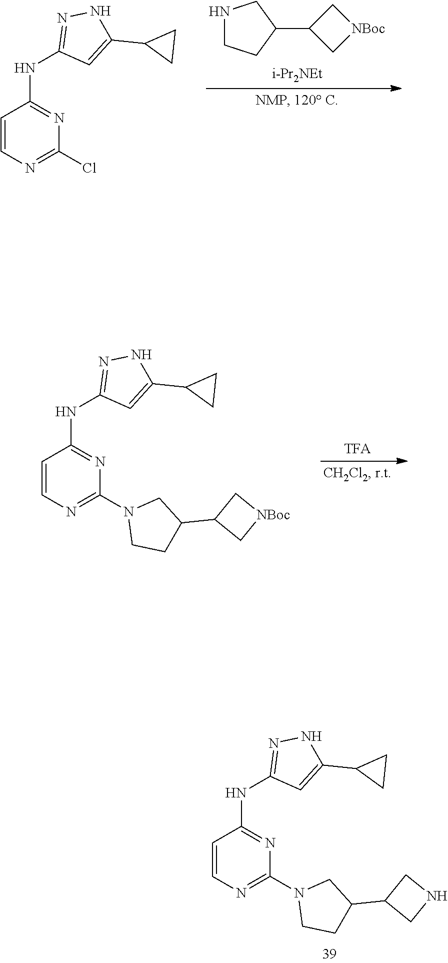 [SMILES: "i-Pr2NEt" ; Clc1nccc(Nc2cc(C3CC3)[nH]n2)n1 ; CC(C)(C)OC(=O)N1CC(C2CCNC2)C1 ; CC(C)(C)OC(=O)N1CC(C2CCN(c3nccc(Nc4cc(C5CC5)[nH]n4)n3)C2)C1 ; c1cc(Nc2cc(C3CC3)[nH]n2)nc(N2CCC(C3CNC3)C2)n1]

### (±)-tert-Butyl 3-[1-[4-[(5-Cyclopropyl-1H-pyrazol-3-yl)amino]pyrimidin-2-yl]pyrrolidin-3-yl]azetidine-1-carboxylate

A solution of 2-chloro-N-(5-cyclopropyl-1H-pyrazol-3-yl)pyrimidin-4-amine (306 mg, 1.30 mmol), tert-butyl 3-(pyrrolidin-3-yl)azetidine-1-carboxylate (348 mg, 1.54 mmol) and N,N-diisopropylethylamine (0.57 mL, 3.3 mmol) in NMP (6 mL) was heated to 120° C. and stirred for 24 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (596 mg, 1.22 mmol, 94%).

¹H NMR (400 MHz, chloroform-d) δ 7.97 (d, J=5.7 Hz, 1H), 6.04 (d, J=5.5 Hz, 1H), 5.88 (s, 1H), 4.07-3.96 (m, 2H), 3.76 (dd, J=10.7, 6.7 Hz, 1H), 3.73-3.64 (m, 3H), 3.56 (dt, J=10.9, 7.6 Hz, 1H), 3.16 (dd, J=10.8, 6.6 Hz, 1H), 2.58-2.47 (m, 2H), 2.15-2.05 (m, 1H), 1.86 (tt, J=8.4, 5.1 Hz, 1H), 1.68-1.56 (m, 1H), 1.43 (s, 9H), 1.00-0.88 (m, 2H), 0.77-0.66 (m, 2H).

UPLC-MS (basic method, 2 min): Rt: 1.06 min, m/z: 426.2 [M+H]⁺.

### (±)-2-[3-(azetidin-3-yl)pyrrolidin-1-yl]-N-(5-cyclopropyl-1H-pyrazol-3-yl)pyrimidin-4-amine

To a solution of tert-butyl 3-[1-[4-[(5-cyclopropyl-1H-pyrazol-3-yl)amino]pyrimidin-2-yl]pyrrolidin-3-yl]azetidine-1-carboxylate (106 mg, 0.22 mmol) in CH₂Cl₂ (2 mL) was added trifluoroacetic acid (2.0 mL, 26 mmol). The reaction mixture stirred for 15 minutes at room temperature, then concentrated to dryness. The crude reaction mixture was then loaded on to an SCX-2 cartridge, the cartridge was washed with MeOH then the compound was eluted with 2 M NH₃ in MeOH. The eluent was concentrated under reduced pressure to afford the title compound as a solid (38 mg, 0.12 mmol, 53%).

¹H NMR (400 MHz, DMSO-d₆) δ 11.89 (s, 1H), 9.33 (s, 1H), 7.82 (d, J=5.7 Hz, 1H), 6.19 (s, 2H), 3.74-3.49 (m, 4H), 3.49-3.21 (m, 4H), 3.09-2.96 (m, 1H), 2.07-1.94 (m, 1H), 1.91-1.79 (m, 1H), 1.64-1.43 (m, 1H), 0.98-0.81 (m, 3H), 0.71-0.55 (m, 2H).

¹H NMR (400 MHz, methanol-d₄) δ 7.81 (d, J=5.7 Hz, 1H), 6.28-5.90 (m, 2H), 3.92-3.79 (m, 1H), 3.75-3.55 (m, 3H), 3.55-3.38 (m, 2H), 3.22-3.03 (m, 1H), 2.88-2.74 (m, 1H), 2.68-2.53 (m, 1H), 2.53-2.35 (m, 1H), 2.17-2.01 (m, 1H), 1.92-1.78 (m, 1H), 1.70-1.54 (m, 1H), 0.99-0.84 (m, 2H), 0.70-0.60 (m, 2H).

UPLC-MS (basic method, 4 min): Rt: 1.04 min, m/z: 326.0 [M+H]⁺.

### Example 55: Synthesis of Compound 40

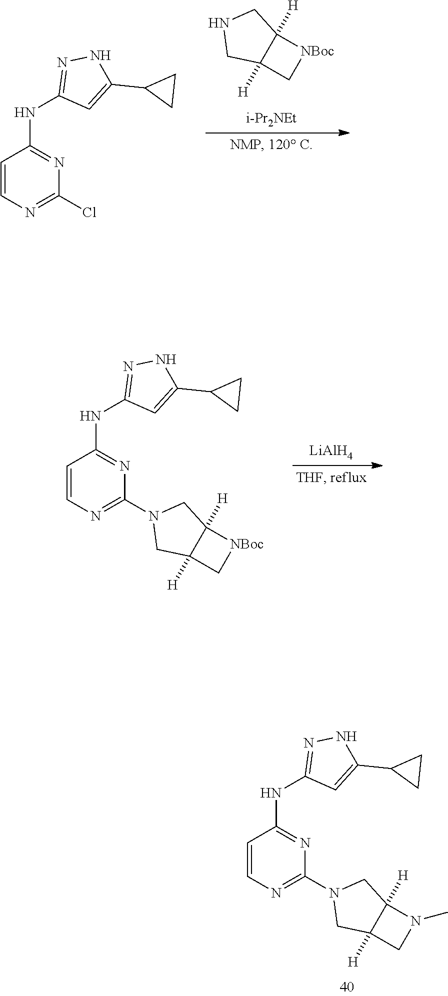 [SMILES: "i-Pr2NEt" ; Clc1nccc(Nc2cc(C3CC3)[nH]n2)n1 ; CC(C)(C)OC(=O)N1C[C@H]2CNC[C@H]21 ; CC(C)(C)OC(=O)N1C[C@H]2CN(c3nccc(Nc4cc(C5CC5)[nH]n4)n3)C[C@H]21 ; "LiAlH4" ; CN1C[C@H]2CN(c3nccc(Nc4cc(C5CC5)[nH]n4)n3)C[C@H]21]

### tert-Butyl (1R,5S)-3-[4-[(5-Cyclopropyl-1H-pyrazol-3-yl)amino]pyrimidin-2-yl]-3,6-diazabicyclo[3.2.0]heptane-6-carboxylate

A solution of 2-chloro-N-(5-cyclopentyl-1H-pyrazol-3-yl)pyrimidin-4-amine (442 mg, 1.68 mmol), (1R,5S)-tert-butyl 3,6-diazabicyclo[3.2.0]heptane-6-carboxylate (379 mg, 1.91 mmol), and N,N-diisopropylethylamine (1.0 mL, 5.9 mmol) in NMP (4 mL) was heated to 120° C. and stirred for 20 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude residue was purified was by automated flash column chromatography, eluting with a gradient of CH₂Cl₂ to 9:1 CH₂Cl₂:2 M NH₃ in MeOH, to afford the title compound as a solid (202 mg, 0.483 mmol, 38%).

¹H NMR (400 MHz, DMSO-d₆) δ 11.92 (s, 1H), 9.42 (s, 1H), 7.88 (d, J=5.7 Hz, 1H), 6.26 (s, 2H), 4.70 (t, J=5.6 Hz, 1H), 4.18 (d, J=12.5 Hz, 1H), 4.02 (d, J=11.7 Hz, 2H), 3.45-3.39 (m, 1H), 3.26-3.16 (m, 1H), 3.13 (d, J=7.4 Hz, 2H), 1.90-1.80 (m, 1H), 1.38 (s, 9H), 0.91 (d, J=8.3 Hz, 2H), 0.67 (d, J=5.5 Hz, 2H).

UPLC-MS (basic method, 4 min): Rt: 1.56 min, m/z: 398.4 [M+H]⁺.

### N-(5-Cyclopropyl-1H-pyrazol-3-yl)-2-[(1S,5S)-6-methyl-3,6-diazabicyclo[3.2.0]heptan-3-yl]pyrimidin-4-amine

To a solution of tert-butyl (1R,5S)-3-[4-[(5-cyclopropyl-1H-pyrazol-3-yl)amino]pyrimidin-2-yl]-3,6-diazabicyclo[3.2.0]heptane-6-carboxylate (189 mg, 0.476 mmol) in anhydrous THE (10 mL) was added a 2.4 M solution of lithium aluminum hydride in THE (2.0 mL, 4.76 mmol) dropwise at 0° C. The reaction mixture was heated to reflux and stirred for 3 hours. The reaction mixture was then cooled to 0° C., then quenched with water (1.8 mL), then a 15% w/v sodium hydroxide solution (1.8 mL), followed by water (5.4 mL). The reaction mixture was warmed to ambient temperature and stirred for 10 minutes, after which magnesium sulfate (ca. 1 g) was added and the reaction mixture was stirred for a further 10 minutes. The reaction mixture was passed through a pad of celite, and the filter cake was washed with EtOAc (20 mL). The filtrate was then concentrated to dryness under reduced pressure. The residue was purified by reversed-phase basic prepHPLC to afford the title compound as a solid (40 mg, 0.127 mmol, 27%).

¹H NMR (400 MHz, DMSO-d₆) δ 11.90 (s, 1H), 9.34 (s, 1H), 7.85 (d, J=5.7 Hz, 1H), 6.24 (s, 2H), 3.87-3.77 (m, 2H), 3.74 (dd, J=6.6, 4.1 Hz, 1H), 3.48 (t, J=10.1 Hz, 1H), 3.11-3.06 (m, 3H), 3.06-2.81 (m, 1H), 2.22 (s, 3H), 1.86 (tt, J=8.8, 5.0 Hz, 1H), 0.93-0.86 (m, 2H), 0.73-0.58 (m, 2H).

UPLC-MS (basic method, 4 min): Rt: 1.16 min, m/z: 312.1 [M+H]⁺.

### Example 56: Synthesis of Compound 44

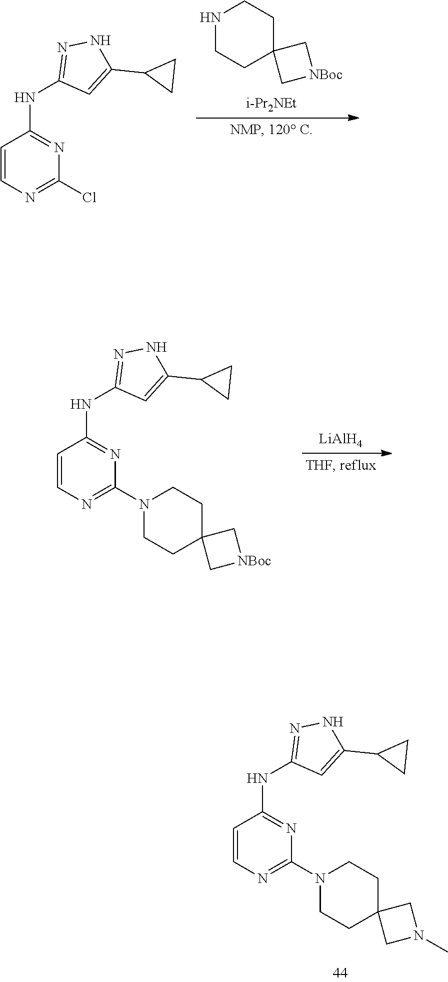

### tert-Butyl 7-[4-[(5-Cyclopropyl-1H-pyrazol-3-yl)amino]pyrimidin-2-yl]-2,7-diazaspiro[3.5]nonane-2-carboxylate

A solution of 2-chloro-N-(5-cyclopropyl-1H-pyrazol-3-yl)pyrimidin-4-amine (200 mg, 0.84 mmol), 2-(tert-butoxycarbonyl)-2,7-diazaspiro[3.5]nonane (288 mg, 1.27 mmol), and N,N-diisopropylethylamine (0.37 ml, 2.12 mmol) in NMP (5 mL) was heated to 120° C. and stirred for 20 hours. The reaction mixture was cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (300 mg, 0.705 mmol, 83%).

¹H NMR (400 MHz, methanol-d₄) δ 7.83 (d, J=5.9 Hz, 1H), 6.15 (s, 1H), 6.03 (s, 1H), 3.69 (s, 8H), 1.96-1.81 (m, 1H), 1.86-1.65 (m, 3H), 1.50-1.33 (m, 10H), 0.96 (d, J=8.5 Hz, 2H), 0.77-0.61 (m, 2H).

UPLC-MS (basic method, 4 min): Rt=1.84 min, m/z=426.3 [M+H]⁺.

### N-(5-Cyclopropyl-1H-pyrazol-3-yl)-2-(2-methyl-2,7-diazaspiro[3.5]nonan-7-yl)pyrimidin-4-amine

To a solution of tert-butyl 7-[4-[(5-cyclopropyl-1H-pyrazol-3-yl)amino]pyrimidin-2-yl]-2,7-diazaspiro[3.5]nonane-2-carboxylate (150 mg, 0.35 mmol) in anhydrous THF (7 mL) was added a 2.4 M solution of lithium aluminum hydride in THF (1.5 mL, 3.5 mmol) dropwise at 0° C. The reaction mixture was heated to reflux and stirred for 3 hours. The reaction mixture was then cooled to 0° C., then quenched with water (1.3 mL), then a 15% w/v sodium hydroxide solution (1.3 mL), followed by water (3.9 mL). The reaction mixture was warmed to ambient temperature and stirred for 10 minutes, after which magnesium sulfate (ca. 1 g) was added and the reaction mixture was stirred for a further 10 minutes. The reaction mixture was passed through a pad of celite, and the filter cake was washed with EtOAc (20 mL). The filtrate was then concentrated to dryness under reduced pressure. The residue was purified by reversed-phase basic prep HPLC to afford the title compound as a solid (22 mg, 63 μmol, 18% yield).

¹H NMR (400 MHz, DMSO-d₆) δ 11.92 (s, 1H), 9.30 (s, 1H), 7.85 (d, J=5.7 Hz, 1H), 6.24 (s, 1H), 6.03 (s, 1H), 3.76-3.51 (m, 4H), 2.94 (s, 4H), 2.23 (s, 3H), 1.92-1.76 (m, 1H), 1.66-1.55 (m, 4H), 1.01-0.77 (m, 2H), 0.83-0.49 (m, 2H).

UPLC-MS (basic method, 4 min): Rt=1.23 min, m/z=340.1 [M+H]⁺.

### Example 57: Synthesis of Compound 45

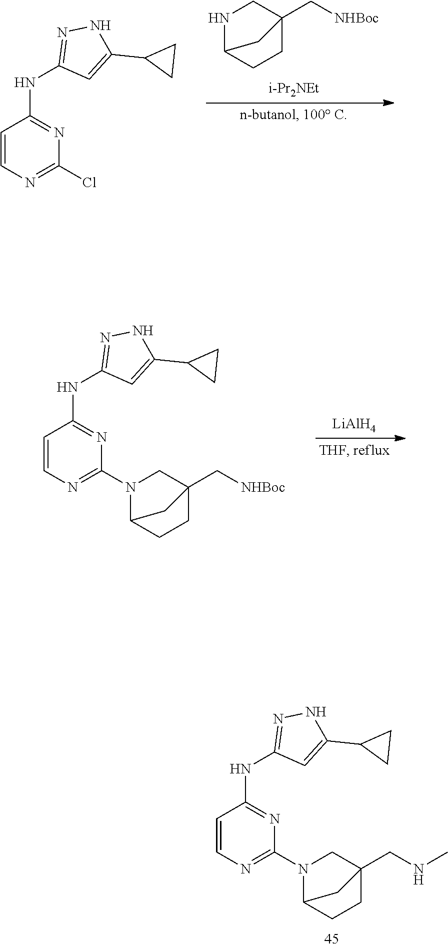 [SMILES: "i-Pr2NEt" ; Clc1nccc(Nc2cc(C3CC3)[nH]n2)n1 ; CC(C)(C)OC(=O)NCC12CCC(C1)NC2 ; CC(C)(C)OC(=O)NCC12CCC(C1)N(c1nccc(Nc3cc(C4CC4)[nH]n3)n1)C2 ; "LiAlH4" ; CNCC12CCC(C1)N(c1nccc(Nc3cc(C4CC4)[nH]n3)n1)C2]

### tert-Butyl N-[[2-[4-[(5-cyclopropyl-1H-pyrazol-3-yl)amino]pyrimidin-2-yl]-2-azabicyclo[2.2.1]heptan-4-yl]methyl]carbamate

A solution of 2-chloro-N-(5-cyclopropyl-1H-pyrazol-3-yl)pyrimidin-4-amine (202 mg, 0.764 mmol), tert-butyl N-({2-azabicyclo[2.2.1]heptan-4-yl}methyl)carbamate (190 mg, 0.840 mmol), and N,N-diisopropylethylamine (0.20 mL, 1.2 mmol) in n-butanol (5 mL) was heated to 100° C. and stirred for 24 hours. The reaction mixture was then concentrated to dryness under reduced pressure. The residue was then suspended in water (20 mL) then extracted with CH₂Cl₂ (3×20 mL). The combined organic extracts were dried over MgSO₄, then concentrated to dryness under reduced pressure. The crude material was purified by normal-phase column chromatography, over silica gel, eluting with a gradient of CH₂Cl₂ to 1:9 2 M NH₃ in MeOH:CH₂Cl₂ to afford the title compound as an oil (232 mg, 0.486 mmol, 64%).

¹H NMR (400 MHz, DMSO-d₆) δ 11.83 (s, 1H), 9.26 (s, 1H), 7.78 (d, J=5.6 Hz, 1H), 7.01 (s, 1H), 6.18 (s, 2H), 4.46 (s, 1H), 3.28 (d, J=5.4 Hz, 2H), 3.20 (d, J=6.2 Hz, 2H), 3.07 (s, 1H), 1.85 (s, 1H), 1.70 (s, 1H), 1.52 (d, J=10.5 Hz, 2H), 1.39 (s, 9H), 1.36 (s, 2H), 0.90 (s, 2H), 0.78-0.56 (m, 2H).

UPLC-MS (basic method, 4 mins): Rt: 1.68 min, m/z=426.4 [M+H]⁺.

### N-(5-Cyclopropyl-1H-pyrazol-3-yl)-2-[4-(methylaminomethyl)-2-azabicyclo[2.2.1]heptan-2-yl]pyrimidin-4-amine

To a solution of tert-butyl N-[[2-[4-[(5-cyclopropyl-1H-pyrazol-3-yl)amino]pyrimidin-2-yl]-2-azabicyclo[2.2.1]heptan-4-yl]methyl]carbamate (230 mg, 0.427 mmol) in anhydrous THE (7 mL) was added a 2.4 M solution of lithium aluminum hydride in THE (1.8 mL, 4.3 mmol) at 0° C. The reaction mixture was then heated to reflux and stirred for 3 hours. The reaction was then cooled to 0° C., then quenched by dropwise addition of water (1.7 mL), followed by a 15% w/v aqueous solution of NaOH (1.7 mL), followed by water (5.1 mL). The reaction mixture was warmed to room temperature and stirred for 15 minutes. MgSO₄ (ca. 1 g) was then added, and the reaction mixture was stirred for 16 hours. The reaction mixture was filtered through a pad of celite, then the filter cake was washed with EtOAc (20 mL), then the filtrate was concentrated to dryness under reduced pressure. The crude residue was purified by reverse-phase prepHIPLC to afford the titled compound as a solid (55 mg, 0.16 mmol, 38%).

¹H NMR (400 MHz, DMSO-d₆) δ 11.88 (s, 1H), 9.26 (s, 1H), 7.79 (d, J=5.7 Hz, 1H), 6.17 (s, 2H), 4.48 (s, 1H), 3.14 (s, 1H), 2.71 (s, 2H), 2.35 (s, 3H), 1.93-1.46 (m, 6H), 1.39 (s, 2H), 0.91 (d, J=8.3 Hz, 2H), 0.64 (dt, J=6.8, 3.4 Hz, 2H).

UPLC-MS (basic method, 4 mins): Rt: 1.23 min, m/z=340.3 [M+H]⁺.

### Example 58: Synthesis of Compound 46

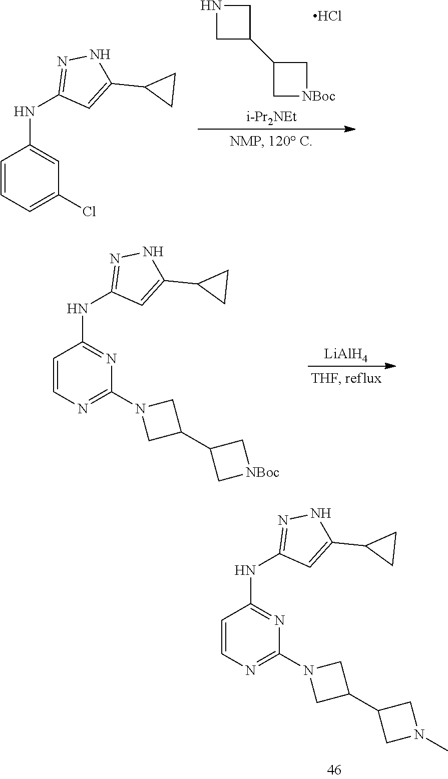

### tert-Butyl 3-[1-[4-[(5-Cyclopropyl-1H-pyrazol-3-yl)amino]pyrimidin-2-yl]azetidin-3-yl]azetidine-1-carboxylate

A solution of 2-chloro-N-(5-cyclopropyl-1H-pyrazol-3-yl)pyrimidin-4-amine (395 mg, 1.68 mmol), tert-butyl [3,3′-biazetidine]-1-carboxylate hydrochloride (500 mg, 2.01 mmol) and N,N-diisopropylethylamine (1.0 mL, 5.9 mmol) in NMP (6 mL) was heated to 120° C. and stirred for 20 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude was purified by flash column chromatography, with a gradient of CH₂Cl₂ to 9:1 CH₂Cl₂:2 M NH₃ MeOH to afford the title compound as a solid (205 mg, 0.478 mmol, 29%).

¹H NMR (400 MHz, DMSO-d₆) δ 11.90 (s, 1H), 9.43 (s, 1H), 7.84 (d, J=5.8 Hz, 1H), 6.48-6.08 (m, 2H), 4.12-4.02 (m, 2H), 3.95 (s, 2H), 3.62 (dd, J=8.6, 4.8 Hz, 2H), 3.53 (s, 2H), 2.99-2.75 (m, 2H), 1.89-1.72 (m, 1H), 1.46-1.29 (m, 9H), 0.98-0.83 (m, 2H), 0.75-0.56 (m, 2H).

UPLC-MS (basic method, 4 min): Rt=1.56 min, m/z=412.5 [M+H]⁺.

### N-(5-Cyclopropyl-1H-pyrazol-3-yl)-2-[3-(1-methylazetidin-3-yl)azetidin-1-yl]pyrimidin-4-amine

To a solution of tert-butyl 3-[1-[4-[(5-cyclopropyl-1H-pyrazol-3-yl)amino]pyrimidin-2-yl]azetidin-3-yl]azetidine-1-carboxylate (65 mg, 0.158 mmol) in THF (10 mL) was added a 2.4 M solution of lithium aluminum hydride in THF (0.65 mL, 1.58 mmol) at 0° C. The reaction mixture was then heated to reflux and stirred for 3 hours. The reaction mixture was allowed to cool to 0° C., then quenched with water (0.60 mL), then a 15% w/v sodium hydroxide solution (0.60 mL), followed by water (1.8 mL). The reaction mixture was warmed to room temperature, stirred for 15 minutes, after which magnesium sulfate (ca. 1 g) was added, and the reaction mixture was stirred for a further 15 minutes. The reaction mixture was passed through a pad of celite, and the filter cake was washed with EtOAc (20 mL). The filtrate was concentrated to dryness under reduced pressure. The residue was purified by reversed-phase basic prepHPLC, to afford the title compound as a solid (22 mg, 67 mmol, 42%).

¹H NMR (400 MHz, DMSO-d₆) δ 11.90 (s, 1H), 9.42 (s, 1H), 7.83 (d, J=6.3 Hz, 1H), 6.53-5.71 (m, 2H), 4.16-3.93 (m, 2H), 3.74-3.55 (m, 2H), 3.28-3.18 (m, 2H), 2.86-2.78 (m, 3H), 2.62 (s, 1H), 2.22-2.15 (m, 3H), 1.91-1.79 (m, 1H), 0.97-0.73 (m, 2H), 0.68-0.57 (m, 2H).

UPLC-MS (basic method, 4 min): Rt=1.16 min, m/z=326.2 [M+H]⁺.

### Example 59: Synthesis of Compound 53

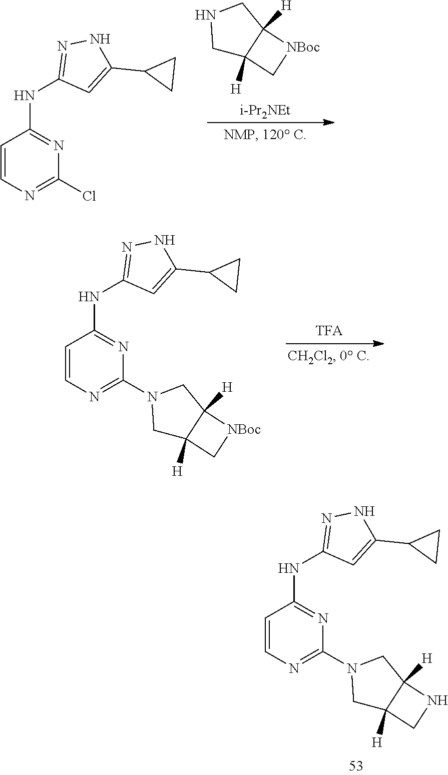

### tert-Butyl (1S,5R)-3-[4-[(5-Cyclopropyl-1H-pyrazol-3-yl)amino]pyrimidin-2-yl]-3,6-diazabicyclo[3.2.0]heptane-6-carboxylate

A solution of 2-chloro-N-(5-cyclopropyl-1H-pyrazol-3-yl)pyrimidin-4-amine (300 mg, 1.27 mmol), tert-butyl (1S,5R)-3,6-diazabicyclo[3.2.0]heptane-6-carboxylate (303 mg, 1.53 mmol) and N,N-diisopropylethylamine (0.55 mL, 3.18 mmol) in NMP (5 mL) was heated to 120° C. and stirred for 20 hours. The reaction mixture was then added dropwise to ice water (ca. 100 mL) resulting in the formation of a precipitate. The precipitate was collected by filtration, then washed with water (2×10 mL). The material was then dried under reduced pressure for 2 hours to afford the title compound as a solid (390 mg, 0.981 mmol, 77%).

¹H NMR (400 MHz, DMSO-d₆) δ 11.91 (s, 1H), 9.41 (s, 1H), 7.87 (d, J=5.8 Hz, 1H), 6.43-6.10 (m, 2H), 4.69 (t, J=5.5 Hz, 1H), 4.18 (d, J=12.5 Hz, 1H), 4.05-3.87 (m, 2H), 3.53-3.36 (m, 1H), 3.25-3.03 (m, 3H), 1.85 (tt, J=8.6, 4.9 Hz, 1H), 1.38 (s, 9H), 0.96-0.86 (m, 2H), 0.74-0.61 (m, 2H).

UPLC-MS (basic method, 2 min): Rt: 1.03 min, m/z=398.2 [M+H]⁺.

### N-(5-Cyclopropyl-1H-pyrazol-3-yl)-2-[(1R,5R)-3,6-diazabicyclo[3.2.0]heptan-3-yl]pyrimidin-4-amine

To a solution of tert-butyl (1S,5R)-3-[4-[(5-cyclopropyl-1H-pyrazol-3-yl)amino]pyrimidin-2-yl]-3,6-di azabicyclo[3.2.0]heptane-6-carboxylate (100 mg, 0.252 mmol) in CH₂Cl₂ (2 mL) was added trifluoroacetic acid (2.0 mL, 26.1 mmol) at room temperature and the reaction mixture was stirred at room temperature for 1 hour. The reaction mixture was then concentrated to dryness under reduced pressure. The crude residue was then loaded onto an SCX-2 cartridge, the cartridge was then washed with water (20 mL), followed by MeOH (20 mL). The compound was eluted with 2 M NH₃ in MeOH (40 mL), and the eluent was concentrated to dryness under reduced pressure to afford the title compound as a solid (54 mg, 0.182 mmol, 72%).

¹H NMR (400 MHz, DMSO-d₆) δ 11.89 (s, 1H), 9.37 (s, 1H), 7.87 (d, J=5.7 Hz, 1H), 6.24 (s, 2H), 4.35 (dd, J=6.6, 4.7 Hz, 1H), 3.92 (dd, J=11.6, 1.4 Hz, 1H), 3.87 (d, J=12.1 Hz, 1H), 3.65 (t, J=7.7 Hz, 1H), 3.30-3.26 (m, 1H), 3.23-3.15 (m, 2H), 3.10 (dd, J=7.9, 4.1 Hz, 1H), 1.86 (tt, J=8.4, 5.1 Hz, 1H), 0.93-0.87 (m, 2H), 0.70-0.62 (m, 2H).

¹H NMR (400 MHz, chloroform-d) δ 8.03 (d, J=5.7 Hz, 1H), 7.16 (s, 1H), 6.11 (d, J=4.5 Hz, 1H), 4.54 (dd, J=6.6, 4.7 Hz, 1H), 4.11 (dd, J=11.5, 1.4 Hz, 1H), 4.06 (d, J=12.3 Hz, 1H), 3.85 (d, J=7.9 Hz, 1H), 3.46 (dd, J=11.3, 7.2 Hz, 1H), 3.40-3.23 (m, 3H), 1.86 (tt, J=8.3, 4.8 Hz, 1H), 0.99-0.90 (m, 2H), 0.76-0.69 (m, 2H).

UPLC-MS (basic method, 4 min): Rt: 0.93 min, m/z=298.1 [M+H]⁺.

### Example 60: Synthesis of Compound 52

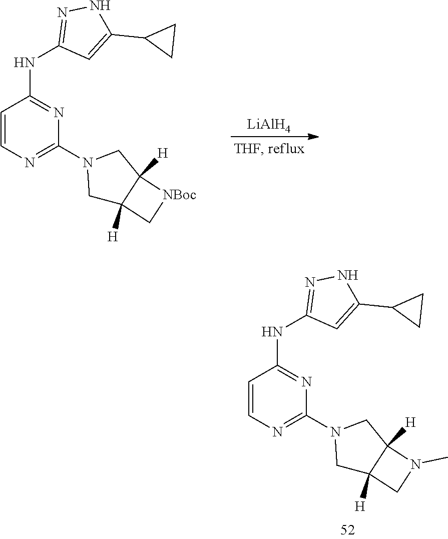 [SMILES: CC(C)(C)OC(=O)N1C[C@@H]2CN(c3nccc(Nc4cc(C5CC5)[nH]n4)n3)C[C@@H]21 ; "LiAlH4" ; CN1C[C@@H]2CN(c3nccc(Nc4cc(C5CC5)[nH]n4)n3)C[C@@H]21]

### N-(5-cyclopropyl-1H-pyrazol-3-yl)-2-[(1R,5R)-6-methyl-3,6-diazabicyclo[3.2.0]heptan-3-yl]pyrimidin-4-amine

To a solution of tert-butyl (1S,5R)-3-[4-[(5-cyclopropyl-1H-pyrazol-3-yl)amino]pyrimidin-2-yl]-3,6-diazabicyclo[3.2.0]heptane-6-carboxylate (144 mg, 0.363 mmol) in anhydrous THF (3 mL) was added a 2.4 M solution of lithium aluminum hydride in THF (1.5 mL, 3.6 mmol) at 0° C., and the reaction mixture was heated to 75° C. for 3 hours. The reaction mixture was allowed to cool to room temperature, then quenched with water (0.15 mL), then a 10% w/v sodium hydroxide solution (0.15 mL), followed by water (0.5 mL). The reaction mixture was stirred for 15 minutes, after which time magnesium sulfate (ca. 1 g) was added and the reaction mixture was stirred for a further 15 minutes. The reaction mixture was passed through a pad of celite, and the filter cake was washed with EtOAc (20 mL). The eluent was concentrated to dryness under reduced pressure to afford N-(5-cyclopropyl-1H-pyrazol-3-yl)-2-[(1R,5R)-6-methyl-3,6-diazabicyclo[3.2.0]heptan-3-yl]pyrimidin-4-amine as a solid (82 mg, 0.25 mmol, 70%).

¹H NMR (400 MHz, DMSO-D₆) δ 11.87 (s, 1H), 9.32 (s, 1H), 7.85 (d, J=5.7 Hz, 1H), 6.22 (s, 2H), 3.84 (dd, J=12.0, 4.3 Hz, 2H), 3.75 (dd, J=6.6, 4.2 Hz, 1H), 3.55-3.43 (m, 1H), 3.13-3.06 (m, 3H), 3.03-2.94 (m, 1H), 2.22 (s, 3H), 1.86 (tt, J=8.4, 5.1 Hz, 1H), 0.98-0.84 (m, 2H), 0.73-0.59 (m, 2H).

UPLC-MS (basic method, 4 min): Rt: 1.17 min, m/z=312.0 [M+H]⁺.

### Example 61: Synthesis of Compound 48

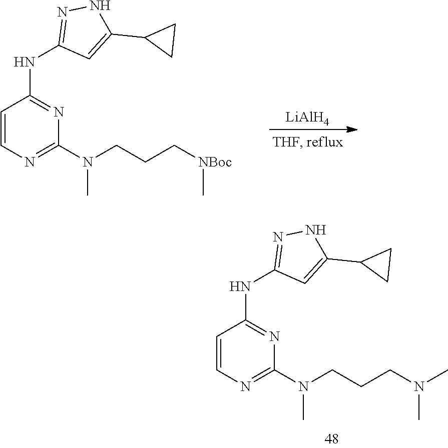 [SMILES: CN(CCCN(C)c1nccc(Nc2cc(C3CC3)[nH]n2)n1)C(=O)OC(C)(C)C ; "LiAlH4" ; CN(C)CCCN(C)c1nccc(Nc2cc(C3CC3)[nH]n2)n1]

### N⁴-(5-cyclopropyl-1H-pyrazol-3-yl)-N²-[3-(dimethylamino)propyl]-N²-methyl-pyrimidine-2,4-diamine

To a solution of tert-butyl N-[3-[[4-[(5-cyclopropyl-1H-pyrazol-3-yl)amino]pyrimidin-2-yl]-methyl-amino]propyl]-N-methyl-carbamate (230 mg, 0.573 mmol) in anhydrous THE (7 mL) was added a 2.4 M solution of lithium aluminum hydride in THE (4.06 mL, 9.74 mmol) at 0° C., and the reaction mixture was heated to reflux and stirred for 3 hours. The reaction mixture was allowed to cool to 0° C., then quenched with water (3.2 mL), then a 15% w/v sodium hydroxide solution (3.2 mL), followed by water (9.8 mL). The reaction mixture was stirred for 15 minutes, after which time magnesium sulfate (ca. 1 g) was added and the reaction mixture was stirred for a further 15 minutes. The reaction mixture was passed through a pad of celite, and the filter cake was washed with EtOAc (20 mL) and the eluent concentrated to dryness. The crude compound was loaded onto an SCX-2 cartridge, the cartridge was then washed with methanol (10 mL), then the compound was eluted with 2 M NH₃ in methanol (20 mL), and the eluent was concentrated to dryness under reduced pressure to afford the title compound as a solid (170 mg, 0.539 mmol, 94%).

¹H NMR (400 MHz, DMSO-d₆) δ 7.82 (d, J=5.6 Hz, 1H), 6.21-6.05 (m, 2H), 3.55 (dd, J=8.4, 6.4 Hz, 2H), 3.04 (s, 3H), 2.21 (t, J=7.1 Hz, 2H), 2.11 (s, 6H), 1.90-1.79 (m, 1H), 1.73-1.60 (m, 2H), 1.00-0.82 (m, 2H), 0.67-0.54 (m, 2H).

UPLC-MS (basic method, 4 min): Rt: 1.25 min, m/z=316.1 [M+H]⁺.

### Example 62: Synthesis of Compound 101

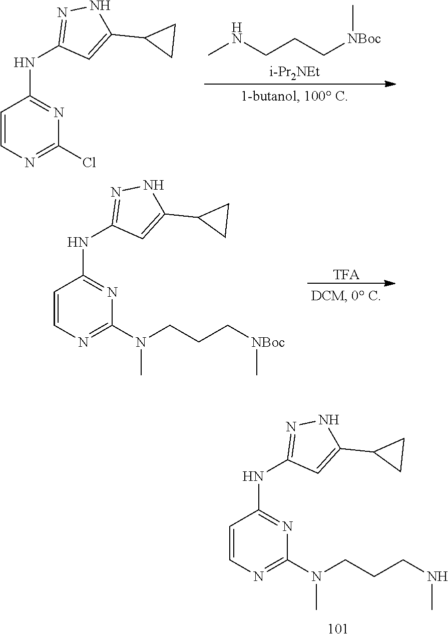

### tert-Butyl N-[3-[[4-[(5-Cyclopropyl-1H-pyrazol-3-yl)amino]pyrimidin-2-yl]-methyl-amino]propyl]-N-methyl-carbamate

A solution of tert-butyl methyl(3-(methylamino)propyl)carbamate (429 mg, 2.12 mmol), 2-chloro-N-(5-cyclopropyl-1H-pyrazol-3-yl)pyrimidin-4-amine (200 mg, 0.849 mmol), and N,N-diisopropylethylamine (0.52 mL, 2.97 mmol) in 1-butanol (7 mL) was heated to 100° C. and stirred for 20 hours. The reaction mixture was left to cool to room temperature and concentrated under reduced pressure. The crude residue was diluted with water (20 mL) and extracted with CH₂Cl₂ (2×20 mL). The organic layer was then washed with brine (50 mL) and dried with MgSO₄ and concentrated under reduced pressure to afford the title compound as an oil (345 mg, 0.791 mmol, 93%).

¹H NMR (400 MHz, methanol-d₄) δ 7.85 (d, J=5.9 Hz, 1H), 6.13 (s, 2H), 3.61 (t, J=7.2 Hz, 2H), 3.27 (t, J=7.7 Hz, 2H), 3.12 (s, 3H), 2.85 (s, 3H), 1.93-1.85 (m, 3H), 1.51-1.37 (m, 9H), 1.01-0.94 (m, 2H), 0.76-0.68 (m, 2H).

UPLC-MS (basic method, 4 min): Rt=1.73 min, m/z=402.3 [M+H]⁺.

### N⁴-(5-Cyclopropyl-1H-pyrazol-3-yl)-N²-methyl-N²-[3-(methylamino)propyl]pyrimidine-2,4-diamine

To a solution of tert-butyl N-[3-[[4-[(5-cyclopropyl-1H-pyrazol-3-yl)amino]pyrimidin-2-yl]-methyl-amino]propyl]-N-methyl-carbamate (115 mg, 0.286 mmol) in CH₂Cl₂ (5 ml), trifluoroacetic acid (5.0 mL, 0.286 mmol) was added dropwise at 0° C., and the reaction was stirred for 30 minutes. The reaction mixture was concentrated to dryness under reduced pressure. The crude compound was loaded onto an SCX cartridge, the cartridge was then washed with methanol, then the compound was eluted with 2 M NH₃ in methanol, and the eluent was concentrated to dryness under reduced pressure to afford the title compound as a solid (50 mg, 0.17 mmol, 58%).

¹H NMR (400 MHz, DMSO-d₆) δ 11.90 (s, 1H), 9.32 (s, 1H), 7.85-7.78 (m, 1H), 6.42-6.07 (m, 2H), 3.61-3.55 (m, 2H), 3.02 (d, J=4.8 Hz, 3H), 2.56-2.52 (m, 2H), 2.29 (d, J=4.3 Hz, 3H), 1.88-1.80 (m, 1H), 1.74-1.68 (m, 2H), 0.93-0.85 (m, 2H), 0.68-0.58 (m, 2H).

UPLC-MS (basic method, 6 min): Rt=1.68 min, m/z=302.1 [M+H]⁺.

### Example 63: Synthesis of Compound 65

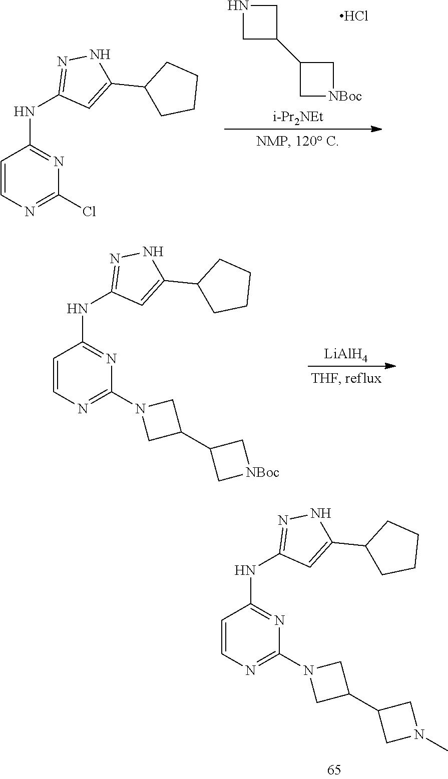

### tert-Butyl 3-[1-[4-[(5-Cyclopentyl-1H-pyrazol-3-yl)amino]pyrimidin-2-yl]azetidin-3-yl]azetidine-1-carboxylate

A solution of 2-chloro-N-(5-cyclopentyl-1H-pyrazol-3-yl)pyrimidin-4-amine (442 mg, 1.68 mmol), tert-butyl [3,3′-biazetidine]-1-carboxylate hydrochloride (500 mg, 2.01 mmol), and N,N-diisopropylethylamine (1.0 mL, 5.9 mmol) in NMP (4 mL) was heated to 120° C. and stirred for 20 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude was purified was by normal-phase flash column chromatography, over silica gel, eluting with a gradient of CH₂Cl₂ to 9:1 CH₂Cl₂:2 M NH₃ in MeOH to afford the title compound as a solid (222 mg, 0.479 mmol, 29%).

¹H NMR (400 MHz, methanol-d₄) δ 7.81 (s, 1H), 6.35 (s, 1H), 6.23 (s, 1H), 4.19 (d, J=8.3 Hz, 2H), 4.06 (d, J=8.2 Hz, 2H), 3.77 (s, 2H), 3.65 (s, 2H), 3.11-3.01 (m, 1H), 2.99-2.85 (m, 2H), 2.05 (s, 2H), 1.89-1.72 (m, 2H), 1.72-1.61 (m, 4H), 1.58-1.25 (m, 9H).

UPLC-MS (basic method, 4 min): Rt: 1.79 min, m/z: 440.4 [M+H]⁺.

### N-(5-Cyclopentyl-1H-pyrazol-3-yl)-2-[3-(1-methylazetidin-3-yl)azetidin-1-yl]pyrimidin-4-amine

To a solution of tert-butyl 3-[1-[4-[(5-cyclopentyl-1H-pyrazol-3-yl)amino]pyrimidin-2-yl]azetidin-3-yl]azetidine-1-carboxylate (199 mg, 0.453 mmol) in anhydrous THE (10 mL) was added a 2.4 M solution of lithium aluminum hydride in THE (1.86 mL, 4.53 mmol) dropwise at 0° C. The reaction mixture was heated to reflux and stirred for 3 hours. The reaction mixture was then cooled to 0° C., then quenched with water (1.6 mL), then a 15% w/v sodium hydroxide solution (1.6 mL), followed by water (3.6 mL). The reaction mixture was warmed to ambient temperature and stirred for 10 minutes, after which magnesium sulfate (ca. 1 g) was added and the reaction mixture was stirred for a further 10 minutes. The reaction mixture was passed through a pad of celite, and the filter cake was washed with EtOAc (20 mL). The filtrate was then concentrated to dryness under reduced pressure. The residue was purified by reversed-phase basic prepHPLC to afford the title compound as a solid (37 mg, 0.11 mmol, 23%).

¹H NMR (400 MHz, DMSO-d₆) δ 11.88 (s, 1H), 9.41 (s, 1H), 7.84 (d, J=5.7 Hz, 1H), 6.28 (s, 2H), 4.03 (d, J=8.3 Hz, 2H), 3.66 (dd, J=8.5, 5.4 Hz, 2H), 3.25 (t, J=7.3 Hz, 2H), 3.08-2.92 (m, 1H), 2.88-2.76 (m, 3H), 2.64-2.58 (m, 1H), 2.18 (s, 3H), 2.06-1.91 (m, 2H), 1.72-1.48 (m, 6H).

UPLC-MS (basic method, 4 min): Rt: 1.34 min, m/z: 354.1 [M+H]⁺.

### Example 64: Synthesis of Compound 67

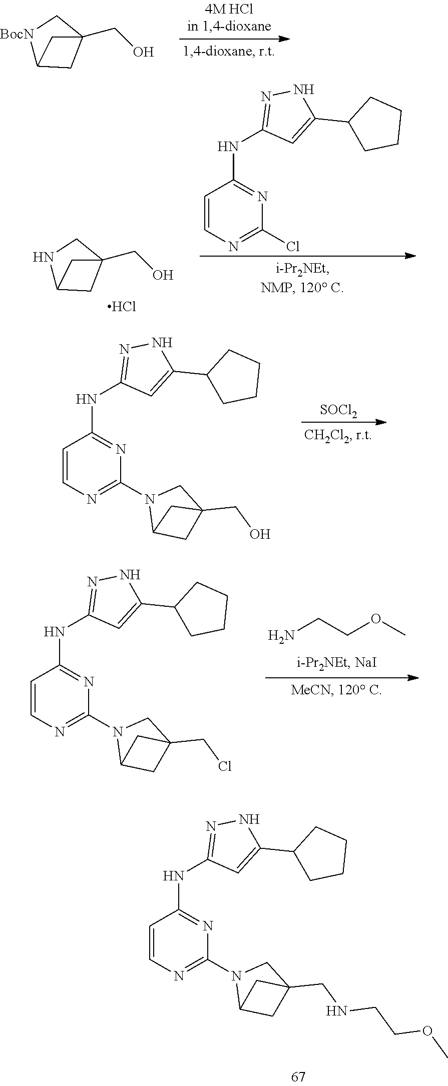

### 2-Azabicyclo[2.1.1]hexan-4-ylmethanol;hydrochloride

To a solution of tert-butyl 4-(hydroxymethyl)-2-azabicyclo[2.1.1]hexane-2-carboxylate (1.00 g, 4.69 mmol) in 1,4-dioxane (20 mL) was added a 4 M solution of hydrogen chloride in 1,4-dioxane (10 mL, 40 mmol) at room temperature and the reaction was stirred for 6 hours. The reaction mixture was then concentrated to dryness under reduced pressure to afford the title compound as a semi-solid (672 mg, 4.49 mmol, 98%).

¹H NMR (400 MHz, DMSO-d₆) δ 9.39 (s, 2H), 4.08 (t, J=1.6 Hz, 1H), 3.60 (s, 2H), 3.10-3.01 (m, 2H), 1.86-1.78 (m, 2H), 1.51-1.43 (m, 2H).

UPLC-MS (basic method, 2 min): Rt: 0.38 min, m/z=114.0 [M−Cl]⁺.

### [2-[4-[(5-Cyclopentyl-1H-pyrazol-3-yl)amino]pyrimidin-2-yl]-2-azabicyclo[2.1.1]hexan-4-yl]methanol

A solution of 2-azabicyclo[2.1.1]hexan-4-ylmethanol;hydrochloride (722 mg, 4.82 mmol), 2-chloro-N-(5-cyclopentyl-1H-pyrazol-3-yl)pyrimidin-4-amine (1.06 g, 4.02 mmol) and N,N-diisopropylethylamine (3.2 mL, 18 mmol) in NMP (10 mL) was heated to 120° C. in a sealed tube, and stirred for 48 hours. The reaction mixture was cooled to room temperature, then loaded onto a 20 g SCX-2 cartridge. The SCX cartridge was washed with water (100 mL), followed by methanol (100 mL). All eluents from both washings were combined then concentrated under reduced pressure. The crude material was purified by automated flash column chromatography, over C18-modified silica, eluting with a gradient of 1:19 MeCN:H₂O with 0.1% v/v NH₃ to 19:1 MeCN:H₂O with 0.1% v/v NH₃ to afford the title compound as a solid (950 mg, 2.79 mmol, 69%).

¹H NMR (400 MHz, DMSO-d₆) δ 11.90 (s, 1H), 9.35 (s, 1H), 7.84 (d, J=5.7 Hz, 1H), 6.48-6.10 (m, 2H), 4.70 (d, J=1.7 Hz, 1H), 4.67 (t, J=5.4 Hz, 1H), 3.64 (d, J=5.3 Hz, 2H), 3.32 (s, 2H), 3.01 (p, J=8.0 Hz, 1H), 2.05-1.94 (m, 2H), 1.82-1.76 (m, 2H), 1.75-1.52 (m, 6H), 1.39-1.30 (m, 2H).

UPLC-MS (basic method, 2 min): Rt: 0.90 min, m/z=341.3 [M+H]⁺.

### 2-[4-(Chloromethyl)-2-azabicyclo[2.1.1]hexan-2-yl]-N-(5-cyclopentyl-1H-pyrazol-3-yl)pyrimidin-4-amine

To a solution of [2-[4-[(5-cyclopentyl-1H-pyrazol-3-yl)amino]pyrimidin-2-yl]-2-azabicyclo[2.1.1]hexan-4-yl]methanol (1.01 g, 2.97 mmol) in CH₂Cl₂ (30 mL) was added thionyl chloride (646 μL, 8.90 mmol) at 0° C. The reaction mixture was warmed to room temperature and stirred for 20 hours. The reaction was quenched with water (50 mL), then extracted with a 1:3 mixture of IPA:CHCl₃ (3×100 mL). The combined organic extracts were dried over Na₂SO₄, then concentrated to dryness under reduced pressure to afford the title compound as a solid (937 mg, 2.61 mmol, 88%).

¹H NMR (400 MHz, DMSO-d₆) δ 11.92 (s, 1H), 9.43 (s, 1H), 7.85 (d, J=5.7 Hz, 1H), 6.47-6.07 (m, 2H), 4.73 (t, J=1.8 Hz, 1H), 3.99 (s, 2H), 3.42 (s, 2H), 3.01 (p, J=7.8 Hz, 1H), 2.03-1.93 (m, 2H), 1.92-1.83 (m, 2H), 1.76-1.53 (m, 6H), 1.50-1.45 (m, 2H).

UPLC-MS (basic method, 2 min): Rt: 1.11 min, m/z=359.3 [M+H]⁺.

### N-(5-Cyclopentyl-1H-pyrazol-3-yl)-2-[4-[(2-methoxyethylamino)methyl]-2-azabicyclo[2.1.1]hexan-2-yl]pyrimidin-4-amine

A suspension of 2-[4-(chloromethyl)-2-azabicyclo[2.1.1]hexan-2-yl]-N-(5-cyclopentyl-1H-pyrazol-3-yl)pyrimidin-4-amine (110 mg, 0.307 mmol), 2-methoxyethylamine (266 μL, 3.07 mmol), sodium iodide (46 mg, 0.307 mmol) and N,N-diisopropylethylamine (160 μL, 0.92 mmol) in anhydrous MeCN (5 mL) was heated to 120° C. in a sealed tube and stirred for 20 h. The reaction mixture was diluted with a 0.5 M aqueous NaOH solution (10 mL) then extracted with a 3:1 mixture of CHCl₃:IPA (3×25 mL). The combined organic extracts were dried over Na₂SO₄, then concentrated to dryness under reduced pressure. The crude residue was dissolved in MeOH (2 mL) then loaded onto a 5 g SCX-2 cartridge. The cartridge was flushed with MeOH (10 mL), then the compound was eluted with 2 M NH₃ in MeOH (25 mL). The eluent was concentrated to dryness under reduced pressure. The crude residue was purified by reversed-phase basic prepHPLC to afford the title compound as a solid (65 mg, 0.16 mmol, 53%).

¹H NMR (400 MHz, DMSO-d₆) δ 8.00 (d, J=5.7 Hz, 1H), 6.87 (s, 1H), 6.12 (s, 1H), 6.03 (s, 1H), 4.84 (t, J=1.8 Hz, 1H), 3.53-3.50 (m, 2H), 3.49 (s, 2H), 3.36 (s, 3H), 3.05 (p, J=8.1 Hz, 1H), 2.95 (s, 2H), 2.87-2.79 (m, 2H), 2.14-2.03 (m, 2H), 1.93-1.85 (m, 2H), 1.84-1.61 (m, 6H), 1.60-1.52 (m, 2H).

UPLC-MS (basic method, 4 min): Rt: 1.58 min, m/z=398.3 [M+H]⁺.

### Example 65: Synthesis of Compound 68

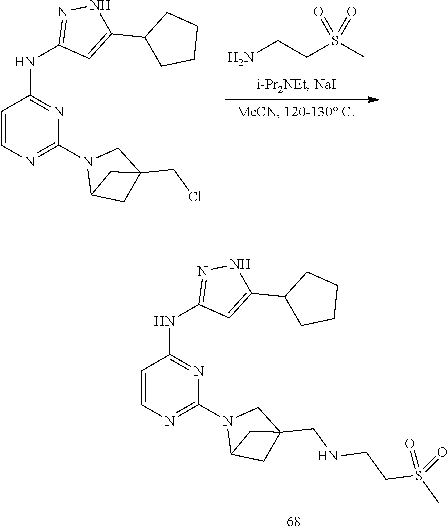

### N-(5-Cyclopentyl-1H-pyrazol-3-yl)-2-[4-[(2-methylsulfonylethylamino)methyl]-2-azabicyclo[2.1.1]hexan-2-yl]pyrimidin-4-amine

A suspension of 2-[4-(chloromethyl)-2-azabicyclo[2.1.1]hexan-2-yl]-N-(5-cyclopentyl-1H-pyrazol-3-yl)pyrimidin-4-amine (150 mg, 0.418 mmol), 2-(methylsulfonyl)ethylamine hydrochloride (334 mg, 2.09 mmol), N,N-diisopropylethylamine (0.58 mL, 3.3 mmol) and sodium iodide (63 mg, 0.42 mmol) in anhydrous MeCN (3 mL) was heated to 120° C. in a sealed tube and stirred for 24 hours. The reaction was then heated to 130° C. and stirred for a further 24 hours. The crude reaction mixture was loaded onto a 5 g SCX-2 cartridge and the cartridge was washed with water (20 mL) then 2 M NH₃ in MeOH (20 mL). Both washings were combined and concentrated to dryness under reduced pressure. The crude material was diluted with a 0.5 M aqueous solution of NaOH (20 mL) then extracted with 3:1 CHCl₃:IPA (3×20 mL). The combined organic extracts were dried over Na₂SO₄, then concentrated to dryness under reduced pressure. The crude material was purified by reversed-phase basic prepHPLC to afford the title compound as a solid (65 mg, 0.15 mmol, 35%).

¹H NMR (400 MHz, DMSO-d₆) δ 11.90 (s, 1H), 9.34 (s, 1H), 7.83 (d, J=6.1 Hz, 1H), 6.51-6.10 (m, 2H), 4.69 (t, J=1.6 Hz, 1H), 3.23 (t, J=6.7 Hz, 2H), 3.06-2.98 (m, 4H), 2.98-2.90 (m, 2H), 2.85-2.78 (m, 2H), 2.04-1.92 (m, 3H), 1.82-1.77 (m, 2H), 1.74-1.52 (m, 6H), 1.41-1.34 (m, 2H).

1H NMR (400 MHz, chloroform-d) δ 8.00 (d, J=5.7 Hz, 1H), 6.87 (s, 1H), 6.15 (s, 1H), 6.01 (s, 1H), 4.85 (s, 1H), 3.47 (s, 2H), 3.24-3.13 (m, 4H), 3.10-3.02 (m, 1H), 3.02 (s, 3H), 2.95 (s, 2H), 2.13-2.02 (m, 2H), 1.89-1.83 (m, 2H), 1.82-1.62 (m, 6H), 1.58-1.53 (m, 2H).

UPLC-MS (basic method, 4 min): Rt: 1.37 min, m/z=446.3 [M+H]⁺.

### Example 66: Synthesis of Compound 69

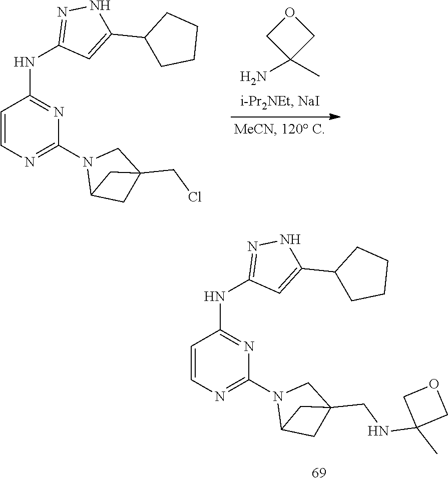

### N-(5-Cyclopentyl-1H-pyrazol-3-yl)-2-[4-[[(3-methyloxetan-3-yl)amino]methyl]-2-azabicyclo[2.1.1]hexan-2-yl]pyrimidin-4-amine

A suspension of 2-[4-(chloromethyl)-2-azabicyclo[2.1.1]hexan-2-yl]-N-(5-cyclopentyl-1H-pyrazol-3-yl)pyrimidin-4-amine (140 mg, 0.390 mmol), 3-methyl-3-oxetanamine (175 μL, 3.90 mmol), N,N-diisopropylethylamine (0.20 mL, 1.2 mmol) and sodium iodide (58 mg, 0.39 mmol) in anhydrous MeCN (3 mL) was heated to 120° C. in a sealed tube and stirred for 20 hours. The reaction mixture was then diluted with a 0.5 M aqueous solution of NaOH (20 mL), then extracted with 1:3 IPA:CHCl₃ (3×20 mL). The combined organic extracts were dried over Na₂SO₄, then concentrated to dryness under reduced pressure. The crude material was purified by reversed-phase basic prepHPLC to afford the title compound as a solid (62 mg, 0.15 mmol, 39%).

¹H NMR (400 MHz, DMSO-d₆) δ 11.88 (s, 1H), 9.34 (s, 1H), 7.83 (d, J=5.7 Hz, 1H), 6.59-6.04 (m, 2H), 4.69 (s, 1H), 4.45 (d, J=5.7 Hz, 2H), 4.19 (d, J=5.7 Hz, 2H), 3.36 (s, 2H), 3.00 (p, J=7.9 Hz, 1H), 2.81 (d, J=7.3 Hz, 2H), 2.17 (t, J=7.5 Hz, 1H), 2.03-1.93 (m, 2H), 1.83-1.76 (m, 2H), 1.75-1.51 (m, 6H), 1.41-1.38 (m, 2H), 1.37 (s, 3H).

UPLC-MS (basic method, 4 min): Rt: 1.46 min, m/z=410.3 [M+H]⁺.

### Example 67: Synthesis of Compound 71

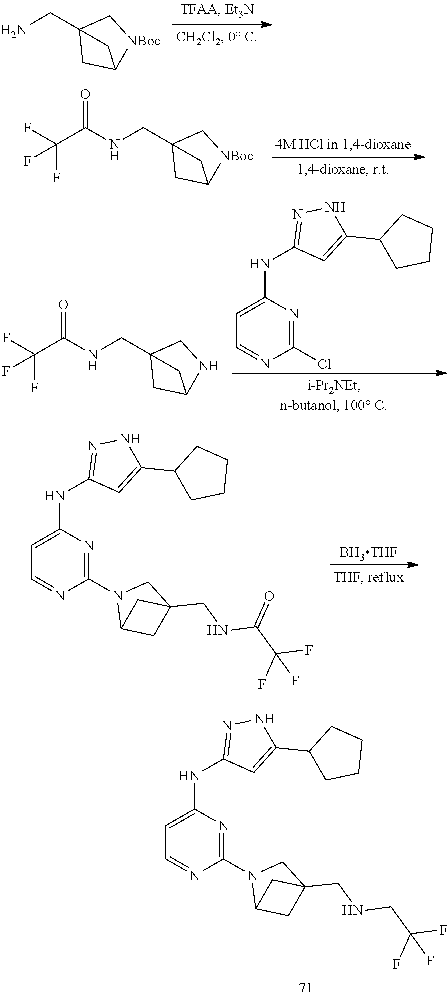

### tert-Butyl 4-[[(2,2,2-Trifluoroacetyl)amino]methyl]-2-azabicyclo[2.1.1]hexane-2-carboxylate

To a solution of tert-butyl 4-(aminomethyl)-2-azabicyclo[2.1.1]hexane-2-carboxylate (620 mg, 2.92 mmol) and triethylamine (0.81 mL, 5.8 mmol) in CH₂Cl₂ (15 mL) was added trifluoroacetic anhydride (0.61 mL, 4.4 mmol) dropwise at 0° C. The reaction mixture was warmed to room temperature and stirred for 22 hours. The reaction mixture was then diluted with CH₂Cl₂ (30 mL), then the organic phase was washed with a saturated aqueous solution of sodium bicarbonate (40 mL) then brine (40 mL). The organic phase was dried over magnesium sulfate, then concentrated to dryness under reduced pressure to afford the title compound as an oil (810 mg, 2.63 mmol, 90%).

¹H NMR (400 MHz, DMSO-d₆) δ 9.59 (t, J=6.2 Hz, 1H), 4.14 (s, 1H), 3.47 (d, J=6.1 Hz, 1H), 3.08 (s, 2H), 1.79-1.61 (m, 2H), 1.39 (s, 9H), 1.30 (s, 2H).

¹⁹F NMR (376 MHz, DMSO-d₆) δ −74.1 (s).

### N-(2-Azabicyclo[2.1.1]hexan-4-ylmethyl)-2,2,2-trifluoro-acetamide;hydrochloride

To a solution of tert-butyl 4-[[(2,2,2-trifluoroacetyl)amino]methyl]-2-azabicyclo[2.1.1]hexane-2-carboxylate (810 mg, 2.63 mmol) in 1,4-dioxane (57 mL) was added a 4 M solution of HCl in 1,4-dioxane (4.5 mL, 18.0 mmol) at 0° C. The reaction mixture was warmed to room temperature and stirred for one hour. The reaction mixture was then concentrated under reduced pressure to afford the title compound as an oil (540 mg, 1.79 mmol, 68%).

¹H NMR (400 MHz, DMSO-d₆) δ 9.72 (t, J=5.8 Hz, 1H), 9.36 (s, 2H), 4.07 (t, J=1.7 Hz, 1H), 3.53 (d, J=6.1 Hz, 2H), 3.06 (dd, J=6.9, 3.3 Hz, 2H), 1.83 (d, J=5.7 Hz, 2H), 1.50 (dd, J=5.6, 2.2 Hz, 2H).

### N-[[2-[4-[(5-Cyclopentyl-1H-pyrazol-3-yl)amino]pyrimidin-2-yl]-2-azabicyclo[2.1.1]hexan-4-yl]methyl]-2,2,2-trifluoro-acetamide

A solution of 2-chloro-N-(5-cyclopentyl-1H-pyrazol-3-yl)pyrimidin-4-amine (290 mg, 1.10 mmol), N-(2-azabicyclo[2.1.1]hexan-4-ylmethyl)-2,2,2-trifluoro-acetamide;hydrochloride (538 mg, 2.20 mmol) and N,N-diisopropylethylamine (0.67 mL, 3.9 mmol) in n-butanol (8 mL) was heated to 100° C. and stirred for 72 hours. The reaction mixture was then cooled to ambient temperature, then concentrated to dryness under reduced pressure. The crude residue was suspended in water (30 mL), then the organics were extracted with EtOAc (3×30 mL). The combined organic extracts were dried over magnesium sulfate, then concentrated to dryness under reduced pressure. The crude residue was purified by normal-phase flash column chromatography, over silica gel, eluting with a gradient of CH₂Cl₂ to 9:1 CH₂Cl₂:2 M NH₃ in MeOH to afford the title compound as an oil (245 mg, 0.484 mmol, 44%).

¹H NMR (400 MHz, DMSO-d₆) δ 11.91 (s, 1H), 9.39 (s, 1H), 8.01-7.59 (m, 1H), 6.28 (s, 2H), 4.68 (s, 1H), 3.55 (d, J=6.0 Hz, 2H), 3.30 (s, 2H), 3.02-2.95 (m, 1H), 2.00-1.93 (m, 2H), 1.79-1.72 (m, 2H), 1.73-1.68 (m, 3H), 1.63-1.55 (m, 4H), 1.41-1.36 (m, 2H).

¹⁹F NMR (376 MHz, DMSO-d₆) δ −74.0 (s).

UPLC-MS (basic method, 4 min): Rt: 1.75 min, m/z=436.2 [M+H]⁺.

### N-(5-Cyclopentyl-1H-pyrazol-3-yl)-2-[4-[(2,2,2-trifluoroethylamino)methyl]-2-azabicyclo[2.1.1]hexan-2-yl]pyrimidin-4-amine

To a solution of N-[[2-[4-[(5-Cyclopentyl-1H-pyrazol-3-yl)amino]pyrimidin-2-yl]-2-azabicyclo[2.1.1]hexan-4-yl]methyl]-2,2,2-trifluoro-acetamide (245 mg, 0.563 mmol) in anhydrous THE (5 mL) was added a 1.0 M solution of borane in THE (5.6 mL, 5.6 mmol) at 0° C. The reaction mixture was heated to reflux and stirred for 20 hours. The reaction was then cooled to ambient temperature, and quenched with MeOH (10 mL), then concentrated to dryness under reduced pressure. The crude reside was then dissolved in MeOH (10 mL), then the solution was concentrated to dryness under reduced pressure. The residue was purified by reversed-phase basic prepHPLC to afford the title compound as a solid (24 mg, 57 mol, 10%).

¹H NMR (400 MHz, DMSO-d₆) δ 11.90 (s, 1H), 9.36 (s, 1H), 7.83 (d, J=5.7 Hz, 1H), 6.29 (s, 2H), 4.69 (s, 1H), 3.35 (s, 2H), 3.31-3.18 (m, 2H), 3.00 (p, J=8.0 Hz, 1H), 2.92 (d, J=6.9 Hz, 2H), 2.43 (p, J=6.5 Hz, 1H), 2.05-1.94 (m, 2H), 1.79 (s, 2H), 1.75-1.52 (m, 6H), 1.47-1.33 (m, 2H).

¹⁹F NMR (376 MHz, DMSO-d₆) δ −70.2-−70.7 (m).

UPLC-MS (basic method, 4 min): Rt: 1.77 min, m/z=422.4 [M+H]⁺.

### Example 68: Synthesis of Compound 72

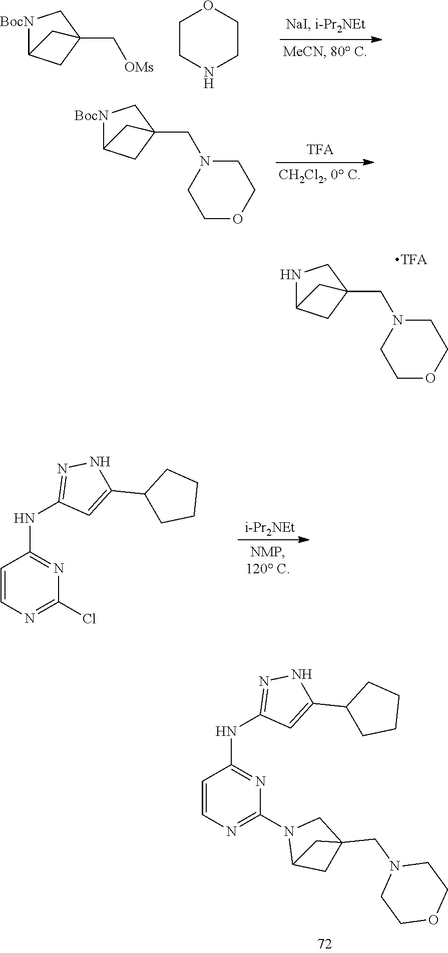

### tert-Butyl 4-(Morpholinomethyl)-2-azabicyclo[2.1.1]hexane-2-carboxylate

To a suspension of 2-methyl-2-propanyl 4-{[(methylsulfonyl)oxy]methyl}-2-azabicyclo[2.1.1]hexane-2-carboxylate (500 mg, 1.72 mmol), sodium iodide (144 mg, 0.958 mmol) and N,N-diisopropylethylamine (0.50 mL, 2.9 mmol) in MeCN (12 mL) was added morpholine (1.5 mL, 17 mmol) dropwise at room temperature. The reaction was heated to 80° C. and stirred 12 hours. The reaction was concentrated to dryness under pressure then suspended in a 10% w/v aqueous solution of NaOH (20 mL) then extracted with EtOAc (3×10 mL). The combined organic extracts were washed with brine (20 mL), dried over MgSO₄ then concentrated to dryness under reduced pressure to afford the title compound as an oil (484 mg, 1.56 mmol, 91%).

¹H NMR (400 MHz, DMSO-d₆) δ 4.17 (t, J=1.8 Hz, 1H), 3.55 (d, J=4.7 Hz, 4H), 3.52-3.47 (m, 1H), 3.22-3.16 (m, 1H), 2.71-2.66 (m, 1H), 2.55 (s, 2H), 2.38-2.30 (m, 4H), 1.75 (dt, J=4.0, 1.8 Hz, 2H), 1.40 (s, 9H), 1.37 (d, J=1.3 Hz, 1H), 1.36 (d, J=1.8 Hz, 1H).

UPLC-MS (basic method, 4 min, ELS detector): Rt: 1.63 min, m/z=283.1 [M+H]⁺.

### 4-(2-Azabicyclo[2.1.1]hexan-4-ylmethyl)morpholine;2,2,2-trifluoroacetic acid

To a solution of tert-butyl 4-(morpholinomethyl)-2-azabicyclo[2.1.1]hexane-2-carboxylate (480 mg, 1.70 mmol) in CH₂Cl₂ (5 mL) was added trifluoroacetic acid (3.0 mL, 39 mmol) dropwise at 0° C. The reaction was held at 0° C. and stirred for 30 minutes. The reaction was concentrated under reduced pressure and the residue loaded onto an SCX-2 cartridge. The cartridge was then washed with methanol (10 mL), then the compound was eluted with 2 M NH₃ in methanol (10 mL). The eluent was concentrated to dryness under reduced pressure to afford the title compound as an oil (1.00 g, 1.68 mmol, 99%).

¹H NMR (400 MHz, DMSO-d₆) δ 4.14-4.09 (m, 1H), 3.81-3.74 (m, 1H), 3.60 (t, J=4.7 Hz, 5H), 3.16 (s, 2H), 3.14-3.07 (m, 1H), 2.74 (s, 2H), 1.92 (d, J=5.7 Hz, 2H), 1.54 (dd, J=5.6, 2.1 Hz, 2H).

¹⁹F NMR (376 MHz, DMSO-d₆) δ −73.62, −74.88-−74.96 (m).

UPLC-MS (basic method, 4 min, ELS detector): Rt: 0.39 min, m/z=183.1 [M+H]⁺.

### N-(5-Cyclopentyl-1H-pyrazol-3-yl)-2-[4-(morpholinomethyl)-2-azabicyclo[2.1.1]hexan-2-yl]pyrimidin-4-amine

A solution of 2-chloro-N-(5-cyclopropyl-1H-pyrazol-3-yl)pyrimidin-4-amine (536 mg, 2.03 mmol), 4-(2-azabicyclo[2.1.1]hexan-4-ylmethyl)morpholine;2,2,2-trifluoroacetic acid (1.00 g, 1.68 mmol) and N,N-diisopropylethylamine (1.5 mL, 8.53 mmol) in NMP (6 mL) was heated to 120° C. and stirred for 24 hours. The reaction mixture was then cooled to ambient temperature, diluted with water (10 mL) then loaded onto an SCX-2 cartridge. The cartridge was washed with methanol (10 mL), then the compound was eluted with 2 M NH₃ in methanol (10 mL). The eluent was then concentrated to dryness under reduced pressure. The crude residue was purified by reversed-phase prepHPLC to afford the title compound as a solid (140 mg, 0.342 mmol, 14%).

¹H NMR (400 MHz, DMSO-d₆) δ 11.87 (s, 1H), 9.37 (s, 1H), 7.82 (d, J=5.7 Hz, 1H), 6.22 (s, 2H), 4.83-4.50 (m, 1H), 3.56 (t, 4H), 3.39 (s, 2H), 3.12-2.95 (m, 1H), 2.62 (s, 2H), 2.37 (t, J=4.6 Hz, 4H), 2.02-1.91 (m, 2H), 1.84-1.77 (m, 2H), 1.76-1.50 (m, 6H), 1.44 (dd, J=4.3, 1.8 Hz, 2H).

UPLC-MS (basic method, 4 min): Rt: 1.57 min, m/z=410.2 [M+H]⁺.

### Example 69: Synthesis of Compound 73

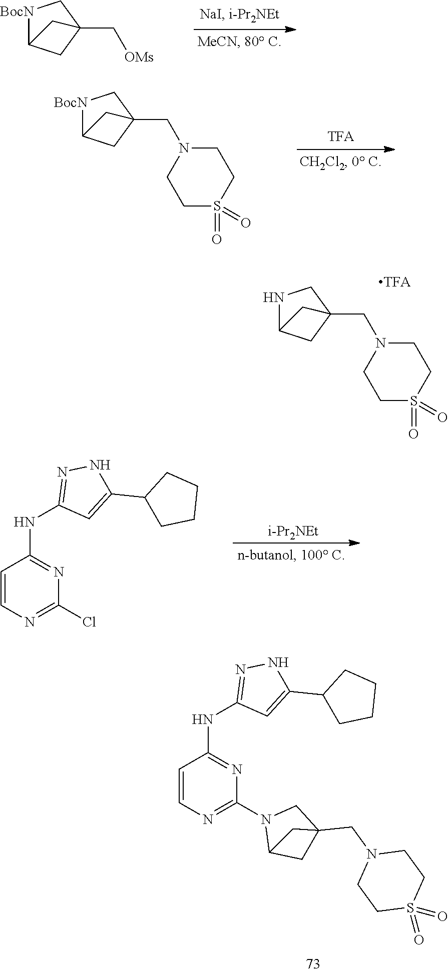

### tert-Butyl 4-[(1,1-Dioxo-1,4-thiazinan-4-yl)methyl]-2-azabicyclo[2.1.1]hexane-2-carboxylate

A suspension of 2-methyl-2-propanyl 4-{[(methylsulfonyl)oxy]methyl}-2-azabicyclo[2.1.1]hexane-2-carboxylate (279 mg, 0.958 mmol), N,N-diisopropylethylamine (0.50 mL, 2.9 mmol), sodium iodide (144 mg, 0.958 mmol), and thiomorpholine 1,1-dioxide (1.29 g, 9.58 mmol) in MeCN (6 mL) was heated to 80° C. and stirred 12 hours. The reaction was concentrated to dryness under pressure then suspended in a 10% w/v aqueous solution of NaOH (20 mL) then extracted with EtOAc (3×10 mL). The combined organic extracts were washed with brine (20 mL), dried over magnesium sulfate then concentrated to dryness under reduced pressure to afford the title compound as an oil (315 mg, 0.953 mmol, 99%).

¹H NMR (400 MHz, methanol-d₄) δ 4.34-4.26 (m, 1H), 3.25-3.20 (m, 3H), 3.11-3.04 (m, 3H), 3.03-2.94 (m, 4H), 2.81 (s, 1H), 1.89-1.81 (m, 2H), 1.53-1.39 (m, 11H), 1.29 (s, 1H).

UPLC-MS (basic method, 2 min, ELS detector): Rt: 1.00 min, m/z=331.1 [M+H]⁺.

### 4-(2-Azabicyclo[2.1.1]hexan-4-ylmethyl)-1,4-thiazinane 1,1-dioxide

To a solution of tert-butyl 4-[(1,1-dioxo-1,4-thiazinan-4-yl)methyl]-2-azabicyclo[2.1.1]hexane-2-carboxylate (315 mg, 0.953 mmol) in CH₂Cl₂ (4 mL) was added trifluoroacetic acid (3.0 mL, 39 mmol) dropwise at 0° C. The reaction was held at 0° C. and stirred for 30 minutes. The reaction was concentrated under reduced pressure and the residue loaded onto an SCX-2 cartridge. The cartridge was then washed with methanol, then the compound was eluted with 2 M NH₃ in methanol. The eluent was concentrated to dryness under reduced pressure to afford the title compound as an oil (215 mg, 0.495 mmol, 52%).

¹H NMR (400 MHz, DMSO-d₆) δ 3.78 (s, 1H), 3.09-3.06 (m, 6H), 2.97-2.93 (m, 2H), 2.91-2.85 (m, 2H), 2.78 (s, 2H), 1.71 (d, J=4.6 Hz, 2H), 1.33 (dd, J=4.6, 1.9 Hz, 2H).

UPLC-MS (basic method, 4 min, ELS detector): Rt: 0.29 min, m/z=231.1 [M+H]⁺.

### N-(5-Cyclopentyl-1H-pyrazol-3-yl)-2-[4-[(1,1-dioxo-1,4-thiazinan-4-yl)methyl]-2-azabicyclo[2.1.1]hexan-2-yl]pyrimidin-4-amine

A solution of 2-chloro-N-(5-cyclopropyl-1H-pyrazol-3-yl)pyrimidin-4-amine (100 mg, 0.379 mmol), 4-(2-azabicyclo[2.1.1]hexan-4-ylmethyl)-1,4-thiazinane 1,1-dioxide (218 mg, 0.948 mmol), and N,N-diisopropylethylamine (0.23 mL, 1.3 mmol) in 1-butanol (6 mL) was heated to 100° C. and the reaction mixture was stirred for 24 hours. The reaction mixture was then cooled to ambient temperature then concentrated to dryness under reduced pressure. The residue was diluted with water (20 mL), then the organics were extracted with CH₂Cl₂ (3×20 mL). The combined organic extracts were dried over magnesium sulfate, then concentrated to dryness under reduced pressure. The crude residue was purified by reverse-phase prepHPLC to afford the title compound as a solid (50 mg, 0.11 mmol, 29%).

¹H NMR (400 MHz, DMSO-d₆) δ 11.90 (s, 1H), 9.38 (s, 1H), 7.83 (d, J=5.7 Hz, 1H), 6.25 (s, 2H), 4.70 (s, 1H), 3.39 (s, 2H), 3.07 (t, J=5.1 Hz, 4H), 3.01 (t, J=7.7 Hz, 1H), 2.92 (dd, J=7.2, 3.5 Hz, 4H), 2.84 (s, 2H), 2.04-1.96 (m, 2H), 1.84-1.78 (m, 2H), 1.76-1.52 (m, 6H), 1.48-1.41 (m, 2H).

UPLC-MS (basic method, 4 min): Rt: 1.48 min, m/z=458.4 [M+H]⁺.

### Example 70: Synthesis of Compound 74

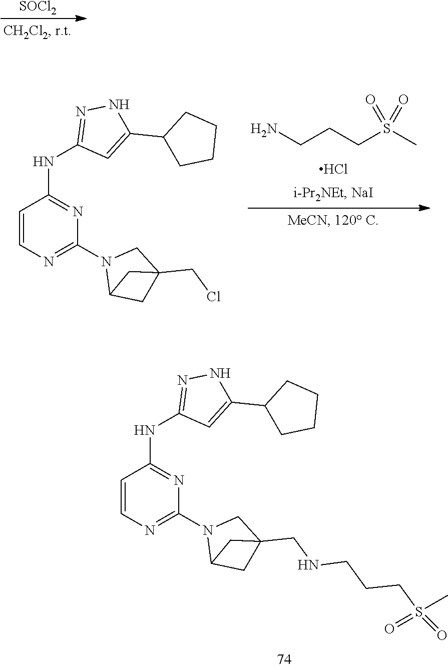

### N-(5-Cyclopentyl-1H-pyrazol-3-yl)-2-[4-[(3-methylsulfonylpropylamino)methyl]-2-azabicyclo[2.1.1]hexan-2-yl]pyrimidin-4-amine

A suspension of 2-[4-(chloromethyl)-2-azabicyclo[2.1.1]hexan-2-yl]-N-(5-cyclopentyl-1H-pyrazol-3-yl)pyrimidin-4-amine (150 mg, 0.418 mmol), 3-methanesulfonyl-propylamine hydrochloride (653 mg, 3.76 mmol), N,N-diisopropylethylamine (0.95 mL, 5.4 mmol) and sodium iodide (63 mg, 0.418 mmol) in anhydrous MeCN (3 mL) was heated to 120° C. in a sealed tube and stirred for 24 hours. The reaction mixture was then diluted with a 0.5 M aqueous NaOH solution (10 mL) then extracted with a 3:1 mixture of CHCl₃:IPA (3×15 mL). The combined organic extracts were dried over Na₂SO₄, then concentrated to dryness under reduced pressure. The crude was then loaded onto a 5 g SCX-2 cartridge, the cartridge was washed with MeOH (10 mL), then the compound was eluted with 2 M NH₃ in MeOH (25 mL). The eluent was concentrated to dryness under reduced pressure. The crude was purified by basic reversed-phase prepHPLC to afford the title compound as a solid (31 mg, 67 μmol, 16%).

¹H NMR (400 MHz, chloroform-d) δ 8.01 (d, J=5.7 Hz, 1H), 6.84 (s, 1H), 6.14 (s, 1H), 6.00 (s, 1H), 4.85 (t, J=1.7 Hz, 1H), 3.47 (s, 2H), 3.19-3.13 (m, 2H), 3.05 (p, J=8.0 Hz, 1H), 2.93 (s, 3H), 2.92 (s, 2H), 2.82 (t, J=6.5 Hz, 2H), 2.13-1.98 (m, 4H), 1.88-1.83 (m, 2H), 1.83-1.62 (m, 6H), 1.59-1.52 (m, 2H).

UPLC-MS (basic method, 4 min): Rt: 1.38 min, m/z=460.3 [M+H]⁺.

### Example 71: Synthesis of Compound 75

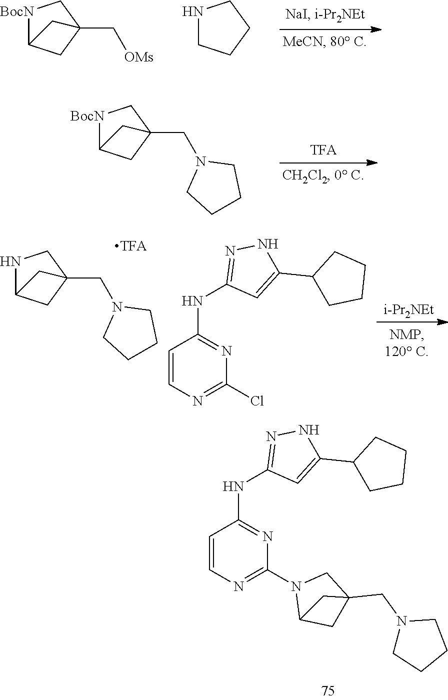

### tert-Butyl 4-(Pyrrolidin-1-ylmethyl)-2-azabicyclo[2.1.1]hexane-2-carboxylate

A suspension of 2-methyl-2-propanyl 4-{[(methylsulfonyl)oxy]methyl}-2-azabicyclo[2.1.1]hexane-2-carboxylate (500 mg, 1.72 mmol), N,N-diisopropylethylamine (0.90 mL, 5.2 mmol), pyrrolidine (1.4 mL, 17 mmol), and sodium iodide (257 mg, 1.72 mmol) in MeCN (20 mL) was heated to 80° C. and stirred for 16 hours. The reaction mixture was then concentrated to dryness under reduced pressure then suspended in a 10% w/v aqueous NaOH solution then extracted with EtOAc (3×10 mL). The combined organic extracts were washed with a saturated aqueous brine solution (20 ml), dried over MgSO₄ the concentrated to dryness under reduced pressure to afford the title compound as an oil (437 mg, 1.64 mmol, 96%).

¹H NMR (400 MHz, methanol-d₄) δ 4.38-4.17 (m, 1H), 3.26 (s, 2H), 2.79 (s, 2H), 2.69-2.29 (m, 4H), 1.87-1.82 (m, 2H), 1.82-1.77 (m, 4H), 1.50-1.47 (m, 1H), 1.47-1.45 (m, 10H).

UPLC-MS (basic method, 4 min, ELS detector): Rt: 1.00 min, m/z=267.1 [M+H]⁺.

### 4-(Pyrrolidin-1-ylmethyl)-2-azabicyclo[2.1.1]hexane;2,2,2-trifluoroacetic acid

To a solution of tert-butyl 4-(pyrrolidin-1-ylmethyl)-2-azabicyclo[2.1.1]hexane-2-carboxylate (437 mg, 1.64 mmol) in CH₂Cl₂ (5 mL) was added trifluoroacetic acid (5.0 mL, 66 mmol) dropwise at 0° C. The reaction was held at 0° C. and stirred for 30 minutes. The reaction was concentrated under reduced pressure and the residue loaded onto an SCX-2 cartridge. The cartridge was then washed with methanol (10 mL), then the compound was eluted with 2 M NH₃ in methanol (10 mL). The eluent was concentrated to dryness under reduced pressure to afford the title compound as an oil (250 mg, 0.634 mmol, 39%).

¹H NMR (400 MHz, methanol-d₄) δ 3.55 (td, J=1.9, 0.6 Hz, 1H), 3.28-3.20 (m, 1H), 2.78 (d, J=0.9 Hz, 2H), 2.76 (s, 2H), 2.55-2.47 (m, 4H), 1.84-1.53 (m, 4H), 1.69-1.59 (m, 2H), 1.39-1.28 (m, 2H).

¹⁹F NMR (376 MHz, methanol-d₄) δ −76.7, −78.8.

UPLC-MS (basic method, 4 min, ELS detector): Rt: 0.66 min, m/z=167.1 [M+H]⁺.

### N-(5-Cyclopentyl-1H-pyrazol-3-yl)-2-[4-(pyrrolidin-1-ylmethyl)-2-azabicyclo[2.1.1]hexan-2-yl]pyrimidin-4-amine

A solution of 2-chloro-N-(5-cyclopentyl-1H-pyrazol-3-yl)pyrimidin-4-amine (110 mg, 0.417 mmol), 4-(pyrrolidin-1-ylmethyl)-2-azabicyclo[2.1.1]hexane;2,2,2-trifluoroacetic acid (247 mg, 0.626 mmol), and N,N-diisopropylethylamine (0.22 mL, 1.25 mmol) in n-butanol (5 mL) was heated to 100° C. and stirred for 24 hours. The reaction mixture was then concentrated to dryness under reduced pressure. The residue was suspended in water (30 ml) then extracted with CH₂Cl₂ (3×30 ml). The combined organic extracts were dried over MgSO₄, then concentrated under reduced pressure. The residue was purified by reverse-phase prepHPLC to afford the title compound as a solid (24 mg, 61 μmol, 15%).

¹H NMR (400 MHz, DMSO-d₆) δ 11.88 (s, 1H), 9.38 (s, 1H), 7.82 (d, J=5.7 Hz, 1H), 6.22 (s, 2H), 4.81-4.55 (m, 1H), 3.50-3.31 (m, 2H), 3.00 (p, J=7.8 Hz, 1H), 2.73 (s, 2H), 2.48-2.41 (m, 4H), 2.03-1.88 (m, 2H), 1.84-1.75 (m, 2H), 1.73-1.49 (m, 10H), 1.47-1.41 (m, 2H).

UPLC-MS (basic method, 4 min): Rt: 1.67 min, m/z=394.2 [M+H]⁺.

### Example 72: Synthesis of Compound 76

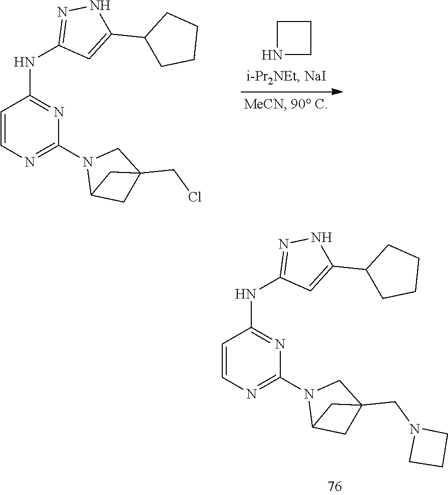

### 2-[4-(Azetidin-1-ylmethyl)-2-azabicyclo[2.1.1]hexan-2-yl]-N-(5-cyclopentyl-1H-pyrazol-3-yl)pyrimidin-4-amine

A suspension of 2-[4-(chloromethyl)-2-azabicyclo[2.1.1]hexan-2-yl]-N-(5-cyclopentyl-1H-pyrazol-3-yl)pyrimidin-4-amine (150 mg, 0.418 mmol), azetidine (0.28 mL, 4.2 mmol) and sodium iodide (63 mg, 0.42 mmol) in anhydrous MeCN (3 mL) was heated to 90° C. in a sealed tube and stirred for 24 hours. The reaction mixture was then diluted with a 0.5 aqueous NaOH solution (10 mL) then extracted with a 3:1 mixture of CHCl₃:IPA (3×15 mL). The combined organic extracts were dried over Na₂SO₄, then concentrated to dryness under reduced pressure. The crude was loaded onto a SCX-2 cartridge, the cartridge was washed with MeOH (10 mL), then the compound was eluted with 2 M NH in MeOH (25 mL). The eluent was concentrated to dryness under reduced pressure. The crude was further purified by basic reverse-phase prepHPLC to afford the title compound as a solid (11 mg, 29 μmol, 7%).

¹H NMR (400 MHz, chloroform-d) δ 8.00 (d, J=5.7 Hz, 1H), 6.80 (s, 1H), 6.11 (d, J=5.7 Hz, 1H), 6.03 (s, 1H), 4.81 (t, J=1.8 Hz, 1H), 3.44 (s, 2H), 3.26 (t, J=7.0 Hz, 4H), 3.05 (p, J=8.0 Hz, 1H), 2.73 (s, 2H), 2.15-2.04 (m, 4H), 1.87-1.82 (m, 2H), 1.82-1.62 (m, 6H), 1.57-1.50 (m, 2H).

UPLC-MS (basic method, 4 min): Rt: 1.74 min, m/z=380.4 [M+H]⁺.

### Example 73: Synthesis of Compound 78

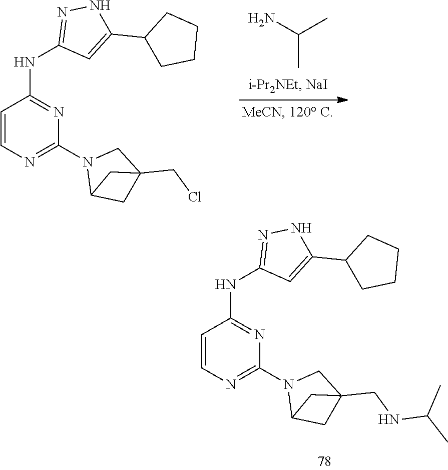

### N-(5-Cyclopentyl-1H-pyrazol-3-yl)-2-[4-[(isopropylamino)methyl]-2-azabicyclo[2.1.1]hexan-2-yl]pyrimidin-4-amine

A suspension of 2-[4-(chloromethyl)-2-azabicyclo[2.1.1]hexan-2-yl]-N-(5-cyclopentyl-1H-pyrazol-3-yl)pyrimidin-4-amine (150 mg, 0.418 mmol), isopropylamine (0.36 mL, 4.2 mmol), N,N-diisopropylethylamine (0.22 mL, 1.3 mmol) and sodium iodide (63 mg, 0.42 mmol) in anhydrous MeCN (3 mL) was heated to 120° C. in a sealed tube and stirred for 20 hours. The reaction mixture was then diluted with a 0.5 M aqueous NaOH solution (20 mL) then extracted with EtOAc (3×20 mL). The combined organic extracts were dried over Na₂SO₄, then concentrated to dryness under reduced pressure. The residue was purified by basic reversed-phase prepHPLC to afford the title compound as a solid (60 mg, 0.16 mmol, 37%).

¹H NMR (400 MHz, DMSO-d₆) δ 11.91 (s, 1H), 9.37 (s, 1H), 7.84 (d, J=5.8 Hz, 1H), 6.28 (s, 2H), 4.83-4.55 (m, 1H), 3.39 (s, 2H), 3.05-2.95 (m, 3H), 2.05-1.93 (m, 2H), 1.86 (s, 2H), 1.75-1.50 (m, 7H), 1.46-1.38 (m, 2H), 1.09 (s, 6H).

UPLC-MS (basic method, 4 min): Rt: 1.59 min, m/z=382.2 [M+H]⁺.

### Example 74: Synthesis of Compound 85

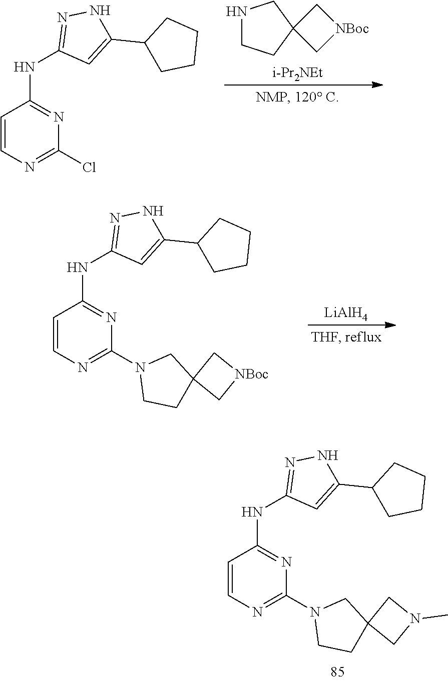

### tert-Butyl 7-[4-[(5-Cyclopentyl-1H-pyrazol-3-yl)amino]pyrimidin-2-yl]-2,7-diazaspiro[3.4]octane-2-carboxylate

A solution of 2-chloro-N-(5-cyclopentyl-1H-pyrazol-3-yl)pyrimidin-4-amine (500 mg, 1.896 mmol) tert-butyl 2,7-diazaspiro[3.4]octane-2-carboxylate (604 mg, 2.84 mmol) and N,N-diisopropylethylamine (0.83 mL, 4.7 mmol) in NMP (10 mL) was heated to 120° C. and stirred for 24 hours. The reaction mixture was then cooled to ambient temperature, then poured into stirring ice water (250 mL), resulting in the formation of a precipitate. The precipitate was collected by filtration, washed with water (2×30 mL) then dried under reduced pressure to afford the title compound as a solid (797 mg, 1.81 mmol, 96%).

¹H NMR (400 MHz, DMSO-d₆) δ 11.83 (s, 1H), 9.32 (s, 1H), 7.79 (d, J=5.7 Hz, 1H), 6.50-6.05 (m, 2H), 3.76 (s, 4H), 3.58 (s, 2H), 3.46 (t, J=6.8 Hz, 2H), 2.97 (dd, J=16.0, 7.3 Hz, 1H), 2.07 (t, J=6.8 Hz, 2H), 1.95 (d, J=7.7 Hz, 2H), 1.66 (d, J=5.8 Hz, 2H), 1.61-1.53 (m, 4H), 1.35 (s, 9H).

UPLC-MS (basic method, 2 min): Rt: 1.14 min, m/z=440.4 [M+H]⁺.

### N-(5-cyclopentyl-1H-pyrazol-3-yl)-2-(2-methyl-2,6-diazaspiro[3.4]octan-6-yl)pyrimidin-4-amine

To a solution of tert-butyl 7-[4[(Cyclopentyl-1H-pyrazol-3-yl)amino]pyrimidin-2-yl]-2,7-diazaspiro[3.4]octane-2-carboxylate (150 mg, 0.341 mmol) in anhydrous THF (10 mL) was added a 2.4 M solution of lithium aluminum hydride in THF (1.4 mL, 3.41 mmol) at 0° C., and then the reaction mixture was heated to reflux for 3 hours. The reaction mixture was allowed to cool to room temperature, then quenched with water (1 mL), then a 15% W/v sodium hydroxide solution (1 mL), followed by water (1 mL). The reaction mixture was stirred for 15 minutes, after which magnesium sulfate (ca. 1 g) was added, and the reaction mixture was stirred for a further 15 minutes. The reaction mixture was passed through a pad of celite, and the filter cake was washed with EtOAc (20 mL). The eluent was concentrated to dryness under reduced pressure. The crude compound was loaded onto an SCX-2 cartridge, the cartridge was then washed with methanol, then the compound was eluted with 2 M NH₃ in methanol, and the eluent was concentrated to dryness under reduced pressure to afford the title compound as a solid (66 mg, 0.18 mmol, 56%).

¹H NMR (400 MHz, DMSO-d₆) 11.87 (s, 1H), 9.36 (s, 1H), 7.82 (d, J=1.7 Hz, 1H), 6.39 (s, 1H), 6.19 (s, 1H), 3.54 (s, 2H), 3.50-3.41 (m, 2H), 3.17-3.11 (m, 2H), 3.06-3.00 (m, 2H), 2.21 (s, 3H), 2.10-2.01 (m, 2H), 2.01-1.93 (m, 2H), 1.76-1.66 (m, 2H), 1.65-1.51 (m, 4H).

¹H NMR (400 MHz, methanol-d₄) δ 7.82 (d, J=6.0 Hz, 1H), 6.40 (s, 1H), 6.14 (s, 1H), 3.64 (s, 2H), 3.59-3.54 (m, 2H), 3.38-3.32 (m, 2H), 3.26 (d, J=7.8 Hz, 2H), 3.10-3.05 (m, 1H), 2.37 (s, 3H), 2.19 (t, J=6.9 Hz, 2H), 2.11-2.04 (m, 2H), 1.83-1.79 (m, 2H), 1.72-1.67 (m, 4H).

UPLC-MS (basic method, 4 min): Rt: 1.42 min, m/z=354.1 [M+H]⁺.

### Example 75: Synthesis of Compound 86

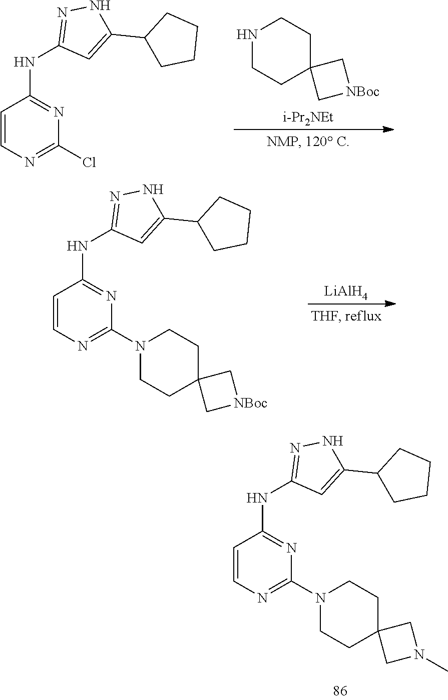

### tert-Butyl 7-[4-[(5-Cyclopentyl-1H-pyrazol-3-yl)amino]pyrimidin-2-yl]-2,7-diazaspiro[3.5]nonane-2-carboxylate

A solution of 2-chloro-N-(5-cyclopentyl-1H-pyrazol-3-yl)pyrimidin-4-amine (150 mg, 0.569 mmol), 2-(tert-butoxycarbonyl)-2,7-diazaspiro[3.5]nonane (193 mg, 0.853 mmol) and N,N-diisopropylethylamine (0.25 mL, 1.4 mmol) in NMP (5 mL) was heated to 120° C. and stirred for 18 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (256 mg, 0.564 mmol, 99%).

¹H NMR (400 MHz, DMSO-d₆) δ 11.91 (s, 1H), 9.33 (s, 1H), 7.86 (d, J=5.6 Hz, 1H), 6.39-6.07 (m, 2H), 3.74-3.62 (m, 4H), 3.00 (p, J=7.8 Hz, 1H), 2.05-1.94 (m, 2H), 1.74-1.67 (m, 2H), 1.67-1.63 (m, 5H), 1.62-1.52 (m, 5H), 1.4-1.35 (m, 11H).

UPLC-MS (basic method, 2 min): Rt: 1.19 min, m/z=454.3 [M+H]⁺.

### N⁴-(5-Cyclopentyl-1H-pyrazol-3-yl)-N²-(2-methyl-2-azaspiro[3.5]nonan-7-yl)pyrimidine-2,4-diamine

To a solution of tert-butyl 7-[4-[(5-cyclopentyl-1H-pyrazol-3-yl)amino]pyrimidin-2-yl]-2,7-diazaspiro[3.5]nonane-2-carboxylate (221 mg, 0.487 mmol) in THE (10 mL) was added a 2.4 M solution of lithium aluminum hydride in THE (2.0 mL, 4.9 mmol) and the reaction mixture heated to reflux and stirred for 2 hours. The reaction mixture was allowed to cool to room temperature, then quenched with water (2 mL), then a 15% w/v sodium hydroxide solution (2 mL), followed by water (2 mL). The reaction mixture was stirred for 15 minutes, after which magnesium sulfate (ca. 1 g) was added, and the reaction mixture was stirred for a further 15 minutes. The reaction mixture was passed through a pad of celite, and the filter cake was washed with EtOAc (20 mL). The eluent was concentrated to dryness under reduced pressure. The crude compound was loaded onto a SCX-2 cartridge, the cartridge was then washed with methanol (10 mL), then the compound was eluted with 2 M NH₃ in methanol (20 mL), and the eluent was concentrated to dryness under reduced pressure to afford the title compound as a solid (102 mg, 0.272 mmol, 56%).

¹H NMR (400 MHz, DMSO-d₆) δ 11.90 (s, 1H), 9.32 (s, 1H), 7.85 (d, J=5.7 Hz, 1H), 6.21 (s, 2H), 3.67-3.60 (m, 4H), 3.02 (q, J=7.9 Hz, 1H), 2.95 (s, 4H), 2.23 (s, 3H), 2.03-1.93 (m, 2H), 1.77-1.49 (m, 10H).

¹H NMR (400 MHz, methanol-d₄) δ 7.81 (dd, J=5.9, 1.0 Hz, 1H), 6.24 (s, 1H), 6.13 (s, 1H), 3.71-3.64 (m, 4H), 3.15 (s, 4H), 3.06 (p, J=8.1 Hz, 1H), 2.36 (s, 3H), 2.12-2.00 (m, 2H), 1.83-1.76 (m, 2H), 1.76-1.72 (m, 4H), 1.71-1.58 (m, 4H).

UPLC-MS (basic method, 4 min): Rt: 1.44 min, m/z=368.2 [M+H]⁺.

### Example 76: Synthesis of Compound 81

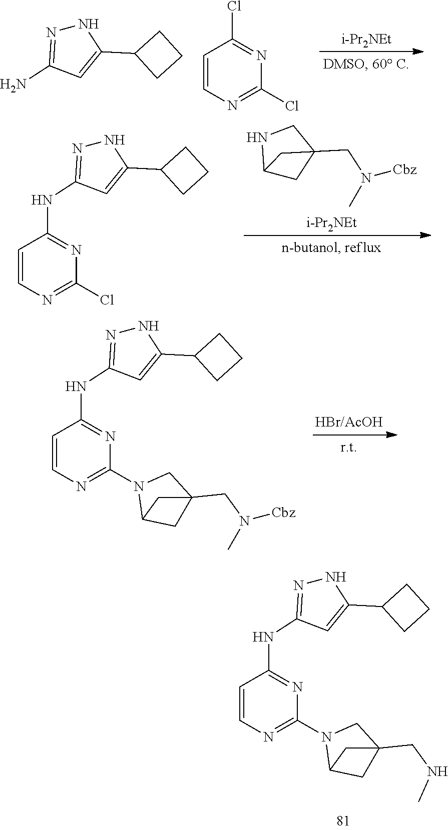

### 2-Chloro-N-(5-cyclobutyl-1H-pyrazol-3-yl)pyrimidin-4-amine

A solution of 2,4-dichloropyrimidine (50 mg, 0.34 mmol), 3-amino-5-cyclobutyl-1H-pyrazole (51 mg, 0.37 mmol) and N,N-diisopropylethylamine (88 μL, 0.50 mmol) in DMSO (2 mL) was heated to 60° C. and stirred for 18 hours. The reaction mixture was then cooled to ambient temperature, then added dropwise to stirring ice water (30 mL), resulting in the formation of a precipitate. The precipitate was collected by filtration, washed with water (2×10 mL) then dried under reduced pressure to afford the title compound as a solid (63 mg, 0.25 mmol, 75%).

¹H NMR (400 MHz, DMSO-d₆) δ 12.22 (s, 1H), 10.31 (s, 1H), 8.16 (s, 1H), 7.41 (s, 1H), 6.06 (s, 1H), 3.49 (p, J=8.6 Hz, 1H), 2.34-2.21 (m, 2H), 2.19-2.04 (m, 2H), 2.02-1.89 (m, 1H), 1.88-1.78 (m, 1H).

UPLC-MS (basic method, 2 min): Rt: 0.96 mins, m/z=250.1 [M+H]⁺.

### Benzyl N-[[2-[4-[(5-Cyclobutyl-1H-pyrazol-3-yl)amino]pyrimidin-2-yl]-2-azabicyclo[2.1.1]hexan-4-yl]methyl]-N-methyl-carbamate

A solution of 2-chloro-N-(5-cyclobutyl-1H-pyrazol-3-yl)pyrimidin-4-amine (120 mg, 0.41 mmol), benzyl N-(2-azabicyclo[2.1.1]hexan-4-ylmethyl)-N-methyl-carbamate (118 mg, 0.46 mmol) and N,N-diisopropylethylamine (0.22 mL, 1.24 mmol) in n-butanol (5 mL) was heated to reflux and stirred for 18 hours. The reaction mixture was then cooled to ambient temperature concentrated under reduced pressure. The residue was purified by normal-phase column chromatography, over silica gel, eluting with a gradient of 2 M NH₃ in MeOH (0% to 10%) in CH₂Cl₂ to afford the title compound as a solid (87 mg, 0.18 mmol, 44%).

¹H NMR (400 MHz, DMSO-d₆) δ 11.89 (s, 1H), 9.33 (s, 1H), 7.80 (d, J=5.7 Hz, 1H), 7.35-7.24 (m, 5H), 6.41-6.11 (m, 2H), 5.04 (d, J=10.3 Hz, 2H), 4.65 (d, J=11.8 Hz, 1H), 3.55 (s, 2H), 3.45-3.36 (m, 1H), 2.89 (d, J=12.7 Hz, 3H), 2.26-2.19 (m, 2H), 2.09-2.04 (m, 3H), 1.95-1.84 (m, 1H), 1.81-1.69 (m, 4H), 1.43-1.33 (m, 2H).

UPLC-MS (basic method, 2 min): Rt: 1.12 min, m/z=474.4 [M+H]⁺.

### N-(5-Cyclobutyl-1H-pyrazol-3-yl)-2-[4-(methylaminomethyl)-2-azabicyclo[2.1.1]hexan-2-yl]pyrimidin-4-amine

To benzyl N-[[2-[4-[(5-cyclobutyl-1H-pyrazol-3-yl)amino]pyrimidin-2-yl]-2-azabicyclo[2.1.1]hexan-4-yl]methyl]-N-methyl-carbamate (87 mg, 0.18 mmol) was added a 30% solution of HBr in acetic acid (3.0 mL) and the reaction mixture stirred at room temperature for 3 hours. The reaction mixture was concentrated to dryness under reduced pressure. The residue was loaded onto an SCX-2 cartridge, then cartridge was washed with MeOH (25 mL), then the compound was eluted with 2 M NH₃ in MeOH (25 mL). The eluent was concentrated to dryness under reduced pressure. The residue was purified by reversed-phase basic prepHPLC to afford the title compound as a solid (29 mg, 90 μmol, 13%).

¹H NMR (400 MHz, DMSO-d₆) δ 11.94 (s, 1H), 9.34 (s, 1H), 7.84 (d, J=5.7 Hz, 1H), 6.28 (s, 2H), 4.69 (t, J=1.7 Hz, 1H), 3.54-3.40 (m, 1H), 3.34 (s, 2H), 2.77 (s, 2H), 2.32 (s, 3H), 2.33-2.21 (m, 2H), 2.18-2.04 (m, 2H), 2.02-1.81 (m, 2H), 1.81-1.78 (m, 2H), 1.40-1.35 (m, 2H).

UPLC-MS (basic method, 4 min): Rt=1.37 mins, m/z=340.2 [M+H⁺].

### Example 77: Synthesis of Compound 103

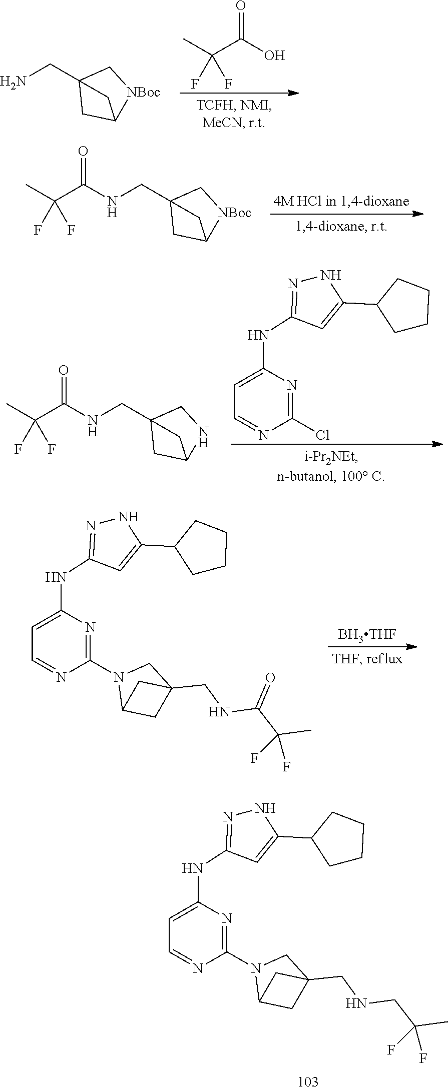

### tert-Butyl 4-[(2,2-difluoropropanoylamino)methyl]-2-azabicyclo[2.1.1]hexane-2-carboxylate

To a solution of 2,2-difluoropropionic acid (400 mg, 3.63 mmol), tert-butyl 4-(aminomethyl)-2-azabicyclo[2.1.1]hexane 2-carboxylate (1.00 g, 4.72 mmol), 1-methylimidazole (1.0 mL, 13 mmol) in MeCN (7 mL) was added chloro-N,N,N′,N′-tetramethylformamidinium hexafluorophosphate (1.22 g, 4.36 mmol) and the reaction mixture was stirred at room temperature for 20 hours. The reaction was then diluted with water (7 mL) then extracted with EtOAc (2×10 mL) the combined organic extracts were washed with water (5 mL), dried over MgSO₄, then concentrated under reduced pressure to afford the title compound as a gum (1.50 g, 3.53 mmol, 97%).

¹H NMR (400 MHz, methanol-d₄) δ 4.29-4.23 (m, 1H), 3.55-3.48 (m, 2H), 2.84-2.72 (m, 2H), 1.81-1.78 (m, 2H), 1.77 (t, J=19.2 Hz, 3H), 1.46 (s, 9H), 1.45-1.37 (m, 2H).

¹⁹F NMR (376 MHz, methanol-d₄) δ −100.9 (q, J=19.2 Hz).

UPLC-MS (basic method, 2 min): Rt: 1.09 min, m/z=303.1 [M−H]⁻.

### N-(2-Azabicyclo[2.1.1]hexan-4-ylmethyl)-2,2-difluoro-propanamide;2,2,2-trifluoroacetic acid

To a solution of tert-butyl 4-[(2,2-difluoropropanoylamino)methyl]-2-azabicyclo[2.1.1]hexane-2-carboxylate (1.51 g, 4.66 mmol) in CH₂Cl₂ (7 mL) was added a trifluoroacetic acid (9.3 mL, 120 mmol) at 0° C. The reaction mixture was warmed to room temperature and stirred for one hour. The reaction mixture was then concentrated to dryness under reduced pressure to afford the title compound as an oil (652 mg, 2.01 mmol, 43%).

¹H NMR (400 MHz, methanol-d₆) δ 4.13 (t, J=1.8 Hz, 1H), 3.58 (s, 2H), 3.35 (s, 1H), 3.25-3.17 (m, 2H), 2.82 (s, 1H), 2.07-1.94 (m, 2H), 1.78 (t, J=19.3 Hz, 3H), 1.65-1.47 (m, 2H).

¹⁹F NMR (376 MHz, methanol-d₄) δ −100.9 (q, J=19.2 Hz).

UPLC-MS (basic method, 2 min): Rt: 0.38 min, m/z=205.1 [M+H]⁺.

### N-[[2-[4-[(5-Cyclopentyl-1H-pyrazol-3-yl)amino]pyrimidin-2-yl]-2-azabicyclo[2.1.1]hexan-4-yl]methyl]-2,2-difluoro-propanamide

A solution of 2-chloro-N-(5-cyclopentyl-1H-pyrazol-3-yl)pyrimidin-4-amine (265 mg, 1.00 mmol), N-(2-azabicyclo[2.1.1]hexan-4-ylmethyl)-2,2-difluoro-propanamide;2,2,2-trifluoroacetic acid (652 mg, 2.01 mmol), and N,N-diisopropylethylamine (0.53 mL, 3.0 mmol) in n-butanol (8 mL) was heated to 100° C. and stirred for 24 hours. The reaction mixture was then concentrated to dryness under reduced pressure. The residue was then suspended in water (20 mL) then extracted with CH₂Cl₂ (3×20 mL), dried over MgSO₄, then concentrated under reduced pressure to afford the title compound as an oil (205 mg, 0.475 mmol, 47%).

¹H NMR (400 MHz, DMSO-d₆) δ 11.89 (s, 1H), 9.35 (s, 1H), 7.83 (d, J=5.7 Hz, 1H), 6.26 (s, 2H), 4.66 (s, 1H), 3.47 (d, J=6.1 Hz, 2H), 3.28 (s, 2H), 3.05-2.92 (m, 1H), 2.73-2.63 (m, 1H), 2.07-1.84 (m, 2H), 1.79-1.68 (m, 7H), 1.64-1.49 (m, 4H), 1.44-1.32 (m, 2H).

¹⁹F NMR (376 MHz, DMSO-d₆) δ −97.4 (q, J=19.3 Hz).

UPLC-MS (basic method, 2 min): Rt: 0.99 min, m/z=432.2 [M+H]⁺.

### N-(5-Cyclopentyl-1H-pyrazol-3-yl)-2-[4-[(2,2-difluoropropylamino)methyl]-2-azabicyclo[2.1.1]hexan-2-yl]pyrimidin-4-amine

To a solution of N-[[2-[4-[(5-cyclopentyl-1H-pyrazol-3-yl)amino]pyrimidin-2-yl]-2-azabicyclo[2.1.1]hexan-4-yl]methyl]-2,2-difluoro-propanamide (202 mg, 0.468 mmol) in anhydrous THF (5 mL) was added a 1.0 M solution of borane-THF complex in THF (4.7 mL, 4.7 mmol) dropwise at 0° C. The reaction mixture was then heated to reflux and stirred for 20 hours. The reaction mixture was cooled to ambient temperature, then quenched with MeOH (10 mL), then concentrated to dryness under reduced pressure. The residue was dissolved in MeOH (10 mL) the concentrated under reduced pressure a further two times. The residue was then purified by reversed-phase basic prepHPLC to afford the title compound as a solid (60 mg, 0.14 mmol, 31%).

¹H NMR (400 MHz, DMSO-d₆) δ 11.89 (s, 1H), 9.35 (s, 1H), 7.82 (d, J=5.7 Hz, 1H), 6.26 (s, 2H), 4.81-4.43 (m, 1H), 3.32 (s, 2H), 3.00 (q, J=8.0 Hz, 1H), 2.94-2.80 (m, 4H), 2.04-1.91 (m, 2H), 1.79 (s, 2H), 1.72-1.66 (m, 2H), 1.66-1.52 (m, 8H), 1.42-1.35 (m, 2H).

¹⁹F NMR (376 MHz, DMSO-d₆) δ −91.8-−94.9 (m).

UPLC-MS (basic method, 4 min): Rt: 1.74 min, m/z=418.4 [M+H]⁺.

### Example 78: Synthesis of Compound 104

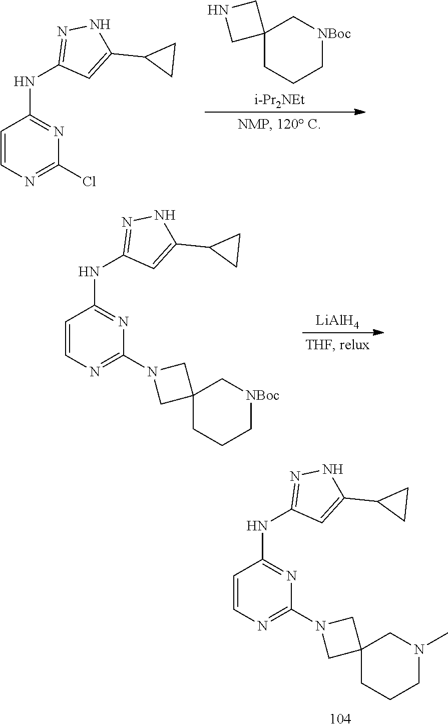 [SMILES: "i-Pr2NEt" ; Clc1nccc(Nc2cc(C3CC3)[nH]n2)n1 ; CC(C)(C)OC(=O)N1CCCC2(CNC2)C1 ; CC(C)(C)OC(=O)N1CCCC2(C1)CN(c1nccc(Nc3cc(C4CC4)[nH]n3)n1)C2 ; "LiAlH4" ; CN1CCCC2(C1)CN(c1nccc(Nc3cc(C4CC4)[nH]n3)n1)C2]

### tert-Butyl 2-[4-[(5-Cyclopropyl-1H-pyrazol-3-yl)amino]pyrimidin-2-yl]-2,8-diazaspiro[3.5]nonane-8-carboxylate

A solution of 2-chloro-N-(5-cyclopropyl-1H-pyrazol-3-yl)pyrimidin-4-amine (340 mg, 1.44 mmol), tert-butyl 2,6-diazaspiro[3.5]nonane-6-carboxylate (490 mg, 2.16 mmol) and N,N-diisopropylethylamine (0.63 mL, 3.6 mmol) in NMP (6 mL) was heated to 120° C. and stirred for 48 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude was purified was by normal-phase flash column chromatography, over silica gel, eluting with a gradient of CH₂Cl₂ to 9:1 CH₂Cl₂:2 M NH₃ in MeOH, to afford the title compound as a solid (200 mg, 0.432 mmol, 30%).

¹H NMR (400 MHz, methanol-d₄) δ 7.82 (s, 1H), 6.21 (s, 2H), 3.81 (d, J=8.4 Hz, 2H), 3.72 (d, J=8.4 Hz, 2H), 3.57 (s, 2H), 3.38 (s, 2H), 1.92-1.79 (m, 3H), 1.50-1.38 (m, 11H), 0.94 (s, 2H), 0.78-0.62 (m, 2H).

UPLC-MS (basic method, 4 min): Rt: 1.72 min, m/z=426.2 [M+H]⁺.

### N-(5-cyclopropyl-1H-pyrazol-3-yl)-2-(8-methyl-2,8-diazaspiro[3.5]nonan-2-yl)pyrimidin-4-amine

To a solution of tert-butyl 2-[4-[(5-cyclopropyl-1H-pyrazol-3-yl)amino]pyrimidin-2-yl]-2,8-diazaspiro[3.5]nonane-8-carboxylate (107 mg, 0.251 mmol) in anhydrous THF (10 mL) was added a 2.4 M solution of lithium aluminum hydride in THE (1.0 mL, 2.51 mmol) dropwise at 0° C. The reaction mixture was heated to reflux and stirred for 2 hours. The reaction mixture was then cooled to 0° C., then quenched with water (0.96 mL), then a 15% w/v sodium hydroxide solution (0.96 mL), followed by water (2.88 mL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (50 mg, 0.147 mmol, 59%).

¹H NMR (400 MHz, DMSO-d₆) δ 12.07 (s, 1H), 9.49 (s, 1H), 7.86 (d, J=6.2 Hz, 1H), 6.36 (s, 1H), 6.22 (s, 1H), 4.09 (s, 1H), 3.79 (s, 4H), 3.04 (s, 1H), 2.77 (s, 4H), 2.03 (s, 1H), 1.92-1.80 (m, 2H), 1.63 (s, 2H), 0.98-0.83 (m, 2H), 0.72-0.57 (m, 2H); upon addition of 1 drop of D₂O to the ¹H NMR sample, followed by re-acquisition of spectrum, the following peak was observed: 3.33 (s, 1H).

UPLC-MS (basic method, 4 min): Rt: 1.35 min, m/z=340.1 [M+H]⁺.

### Example 79: Synthesis of Compound 105

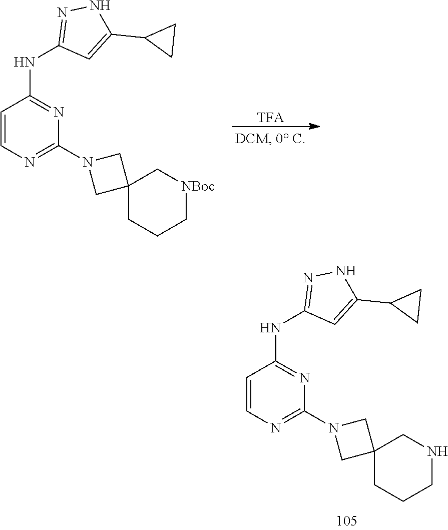 [SMILES: CC(C)(C)OC(=O)N1CCCC2(C1)CN(c1nccc(Nc3cc(C4CC4)[nH]n3)n1)C2 ; c1cc(Nc2cc(C3CC3)[nH]n2)nc(N2CC3(CCCNC3)C2)n1 ; "TFA"]

### N-(5-Cyclopropyl-1H-pyrazol-3-yl)-2-(2,8-diazaspiro[3.5]nonan-2-yl)pyrimidin-4-amine

To a solution of tert-butyl 2-[4-[(5-cyclopropyl-1H-pyrazol-3-yl)amino]pyrimidin-2-yl]-2,8-diazaspiro[3.5]nonane-8-carboxylate (91 mg, 0.214 mmol) in CH₂Cl₂ (5 mL) was added trifluoroacetic acid (4.6 mL, 60 mmol) dropwise at 0° C. The reaction mixture was stirred for 30 mins, then concentrated to dryness under reduced pressure. The crude residue was loaded onto an SCX cartridge, the cartridge was washed with MeOH (10 mL), then the compound was eluted with 2 M NH₃ in MeOH (20 mL). The eluent was concentrated to dryness under reduced pressure. The residue was then dissolved in CH₂Cl₂ (10 mL), then Amberlyst A21 was added, and the suspension was stirred for one hour. The suspension was then filtered, and the filtrate was concentrated to dryness under reduced pressure to afford the title compound as a solid (14 mg, 43 μmol, 20%).

¹H NMR (400 MHz, methanol-d₄) δ 7.83 (d, J=6.0 Hz, 1H), 6.19 (s, 2H), 3.94-3.68 (m, 4H), 2.92 (s, 2H), 2.74 (s, 2H), 1.93-1.80 (m, 3H), 1.59 (s, 2H), 1.30 (s, 1H), 0.97 (d, J=8.2 Hz, 2H), 0.73 (d, J=4.9 Hz, 2H).

UPLC-MS (basic method, 4 min): Rt: 1.15 min, m/z=326.0 [M+H]⁺.

### Example 80: Synthesis of Compound 106

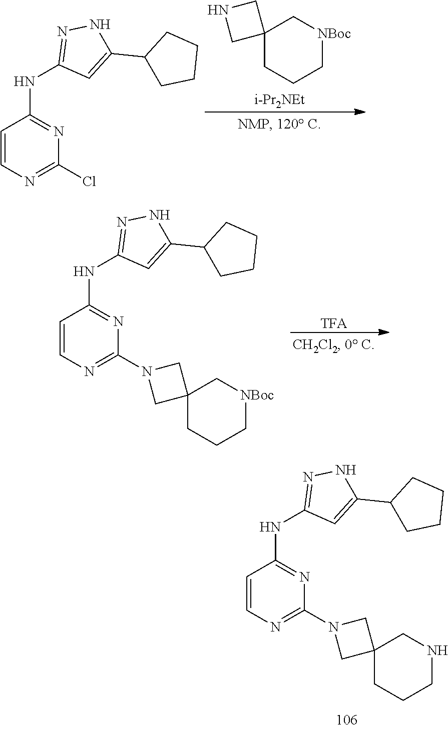

### tert-Butyl 2-[4-[(5-Cyclopentyl-1H-pyrazol-3-yl)amino]pyrimidin-2-yl]-2,8-diazaspiro[3.5]nonane-8-carboxylate

A solution of 2-chloro-N-(5-cyclopentyl-1H-pyrazol-3-yl)pyrimidin-4-amine (386 mg, 1.46 mmol), tert-butyl 2,6-diazaspiro[3.5]nonane-6-carboxylate (497 mg, 2.20 mmol), and N,N-diisopropylethylamine (0.64 mL, 3.7 mmol), in NMP (3.5 mL) was heated to 120° C. and stirred for 72 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The crude was purified was by normal-phase flash column chromatography, over silica gel, eluting with a gradient of CH₂Cl₂ to 9:1 CH₂Cl₂:2 M NH₃ in MeOH, to afford the title compound as an oil (50 mg, 96 μmol, 7%).

¹H NMR (400 MHz, methanol-d₄) δ 7.82 (d, J=6.0 Hz, 1H), 6.37 (s, 1H), 6.22 (s, 1H), 3.81 (d, J=8.4 Hz, 2H), 3.72 (d, J=8.5 Hz, 2H), 3.57 (s, 2H), 3.38 (s, 2H), 3.13-2.99 (m, 1H), 2.10-2.01 (m, 2H), 1.90-1.72 (m, 4H), 1.70-1.63 (m, 4H), 1.56 (s, 2H), 1.45 (s, 9H).

UPLC-MS (basic method, 4 min): Rt: 1.92 min, m/z=454.3 [M+H]⁺.

### N-(5-Cyclopentyl-1H-pyrazol-3-yl)-2-(2,8-diazaspiro[3.5]nonan-2-yl)pyrimidin-4-amine

To a solution of tert-butyl 2-[4-[(5-cyclopentyl-1H-pyrazol-3-yl)amino]pyrimidin-2-yl]-2,8-diazaspiro[3.5]nonane-8-carboxylate (50 mg, 0.11 mmol) in CH₂Cl₂ (3 mL) was added trifluoroacetic acid (3.0 mL, 39 mmol) dropwise at 0° C. The reaction mixture was stirred for 30 mins. The reaction was concentrated to dryness under reduced pressure and the residue dissolved in MeOH (5 mL) then loaded onto an SCX-2 cartridge. The cartridge was washed with MeOH (10 mL) then the compound was eluted with 2 M NH₃ in methanol (20 mL). The eluent was concentrated to dryness under reduced pressure. The residue was dissolved in CH₂Cl₂, and Amberlyst A21 (ca. 100 mg) was added, and the suspension was stirred for 1 hour. The resin was removed by filtration, and the solution was concentrated to dryness under reduced pressure to afford the title compound as a solid (3 mg, 8.3 μmol, 8%).

¹H NMR (400 MHz, DMSO-d₆) δ 7.71 (s, 1H), 6.09 (s, 2H), 3.65 (s, 2H), 3.56 (d, J=8.2 Hz, 2H), 3.04-2.91 (m, 1H), 2.74 (d, J=4.5 Hz, 2H), 2.58 (s, 2H), 2.13 (t, J=4.4 Hz, 1H), 2.01-1.85 (m, 2H), 1.77-1.49 (m, 8H), 1.40 (s, 2H).

UPLC-MS (basic method, 4 min): Rt: 1.37 min, m/z=354.1 [M+H]⁺.

### Example 81: Synthesis of Compound 107

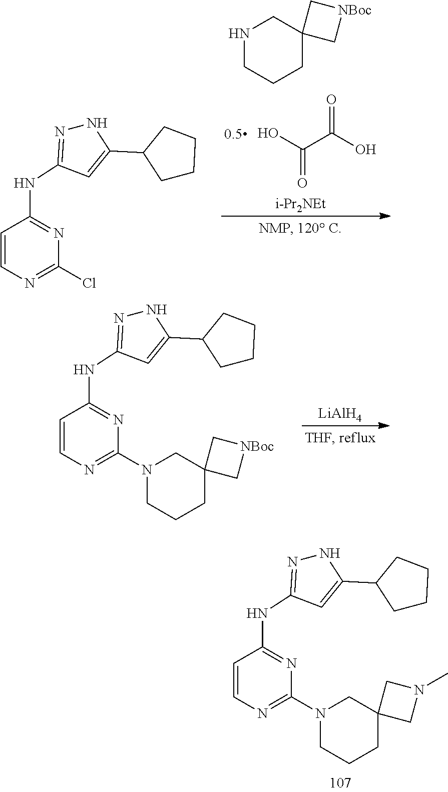

### tert-Butyl 8-[4-[(5-Cyclopentyl-1H-pyrazol-3-yl)amino]pyrimidin-2-yl]-2,8-diazaspiro[3.5]nonane-2-carboxylate

A solution of 2-chloro-N-(5-cyclopentyl-1H-pyrazol-3-yl)pyrimidin-4-amine (325 mg, 1.23 mmol), 2-methyl-2-propanyl 2,6-diazaspiro[3.5]nonane-2-carboxylate ethanedioate (2:1) (1.00 g, 1.85 mmol), and N,N-diisopropylethylamine (0.75 mL, 4.31 mmol) in NMP (4 mL) was heated to 120° C. and stirred for 48 hours. The reaction mixture was then cooled to ambient temperature, then added dropwise to stirring ice-water (100 mL), resulting in the formation of a precipitate. The precipitate was collected by filtration, washed with water (2×10 mL) then dried under reduced pressure to afford the title compound as a solid (305 mg, 0.592 mmol, 48%).

¹H NMR (400 MHz, methanol-d₄) δ 7.83 (d, J=6.0 Hz, 1H), 6.32 (s, 1H), 6.21-6.12 (m, 1H), 3.90 (s, 1H), 3.69-3.41 (m, 5H), 3.15-2.94 (m, 1H), 2.42-2.00 (m, 2H), 1.87-1.58 (m, 10H), 1.45-1.39 (m, 11H).

UPLC-MS (basic method, 4 min): Rt=2.02 min, m/z=454.2 [M+H]⁺.

### N-(5-cyclopentyl-1H-pyrazol-3-yl)-2-(2-methyl-2,6-diazaspiro[3.5]nonan-6-yl)pyrimidin-4-amine

To a solution of tert-butyl 8-[4-[(5-cyclopentyl-1H-pyrazol-3-yl)amino]pyrimidin-2-yl]-2,8-diazaspiro[3.5]nonane-2-carboxylate (100 mg, 0.220 mmol) in anhydrous THF (10 mL) was added a 2.4 M solution of lithium aluminum hydride in THF (0.92 mL, 2.20 mmol) dropwise at 0° C. The reaction mixture was heated to reflux and stirred for 3 hours. The reaction mixture was then cooled to 0° C., then quenched with water (0.92 mL), then a 15% w/v sodium hydroxide solution (0.92 mL), followed by water (2.75 mL). The reaction mixture was warmed to ambient temperature and stirred for 10 minutes, after which magnesium sulfate (ca. 1 g) was added and the reaction mixture was stirred for a further 10 minutes. The reaction mixture was passed through a pad of celite, and the filter cake was washed with EtOAc (20 mL). The filtrate was then concentrated to dryness under reduced pressure. The residue was purified by reversed-phase basic prepHIPLC to afford the title compound as a solid (15 mg, 0.0408 mmol, 19%).

¹H NMR (400 MHz, DMSO-d₆) δ 11.91 (s, 1H), 9.39 (s, 1H), 7.83 (d, J=5.6 Hz, 1H), 6.40 (s, 1H), 6.16 (s, 1H), 3.83 (s, 2H), 3.62 (t, J=5.5 Hz, 2H), 3.05-2.95 (m, 3H), 2.74 (d, J=6.8 Hz, 2H), 2.21 (s, 3H), 1.99 (s, 2H), 1.74-1.54 (m, 8H), 1.46-1.35 (m, 2H).

UPLC-MS (basic method, 4 min): Rt=1.75 min, m/z=368.2 [M+H]⁺, 100% purity.

### Example 82: Synthesis of Compound 108

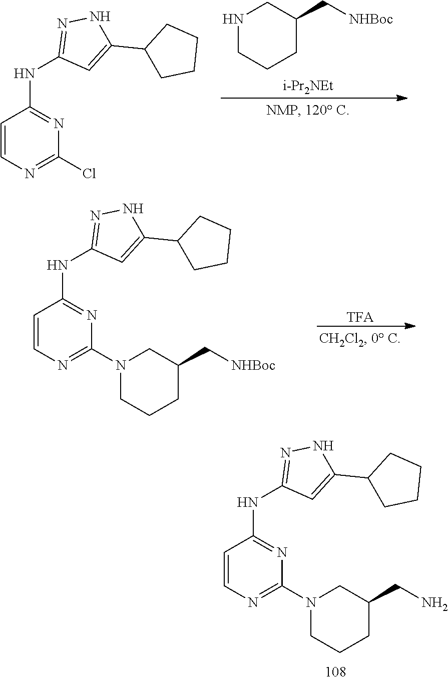

### tert-Butyl N-[[(3R)-1-[4-[(5-Cyclopentyl-1H-pyrazol-3-yl)amino]pyrimidin-2-yl]-3-piperidyl]methyl]carbamate

A solution of 2-chloro-N-(5-cyclopentyl-1H-pyrazol-3-yl)pyrimidin-4-amine (280 mg, 1.06 mmol), N,N-diisopropylethylamine (0.46 mL, 2.7 mmol), and tert-butyl N-[[(3S)-3-piperidyl]methyl]carbamate (341 mg, 1.59 mmol) in NMP (3.6 mL) was heated to 120° C. and stirred for 16 hours. The reaction mixture was added dropwise to ice-water (ca. 150 ml) resulting in a precipitation, then the precipitate was collected by vacuum filtration. The wet filter cake was re-dissolved in CH₂Cl₂ (20 mL), dried over Na₂SO₄, and concentrated to dryness to afford the title compound as a foam (471 mg, 1.05 mmol, 99%).

¹H NMR (400 MHz, DMSO-d₆) δ 11.90 (s, 1H), 9.32 (s, 1H), 7.84 (d, J=5.7 Hz, 1H), 6.86 (t, J=5.8 Hz, 1H), 6.23 (s, 2H), 4.50-4.38 (m, 2H), 3.05-2.95 (m, 1H), 2.91-2.81 (m, 3H), 2.65-2.51 (m, 1H), 2.00 (m, 2H), 1.80-1.50 (m, 9H), 1.30-1.39 (m, 10H), 1.26-1.09 (m, 1H).

UPLC-MS (basic method, 4 min): Rt: 2.03 min, m/z: 442.2 [M+H]⁺.

### 2-[(3R)-3-(Aminomethyl)-1-piperidyl]-N-(5-cyclopentyl-1H-pyrazol-3-yl)pyrimidin-4-amine

To a solution of tert-butyl N-[[(3R)-1-[4-[(5-cyclopentyl-1H-pyrazol-3-yl)amino]pyrimidin-2-yl]-3-piperidyl]methyl]carbamate (471 mg, 1.07 mmol) in CH₂Cl₂ (14 mL) was added trifluoroacetic acid (0.82 mL, 11 mmol) at 0° C. The reaction mixture was warmed to ambient temperature and stirred for 5 hours, after which the reaction mixture was concentrated to dryness under reduced pressure. The crude residue was loaded onto an SCX-2 cartridge, the cartridge was then washed with methanol (100 mL), then the compound was eluted with 2 M NH₃ in methanol (100 mL), and the eluent was concentrated to dryness under reduced pressure to afford the title compound as a solid (294 mg, 0.835 mmol, 78%).

¹H NMR (400 MHz, DMSO-d₆) δ 11.90 (s, 1H), 9.33 (s, 1H), 7.84 (d, J=5.6 Hz, 1H), 6.19-6.26 (m, 2H), 4.51 (m, 1H), 4.42 (m, 1H), 3.17 (s, 1H), 3.06-2.86 (m, 2H), 2.72-2.61 (m, 1H), 2.50-2.38 (m, 2H), 2.04-1.91 (m, 2H), 1.90-1.79 (m, 1H), 1.77-1.50 (m, 7H), 1.45-1.29 (m, 2H), 1.22-1.08 (m, 1H).

UPLC-MS (basic method, 4 min): Rt: 1.58 min, m/z: 342.2 [M+H]⁺.

### Example 83: Synthesis of Compound 109

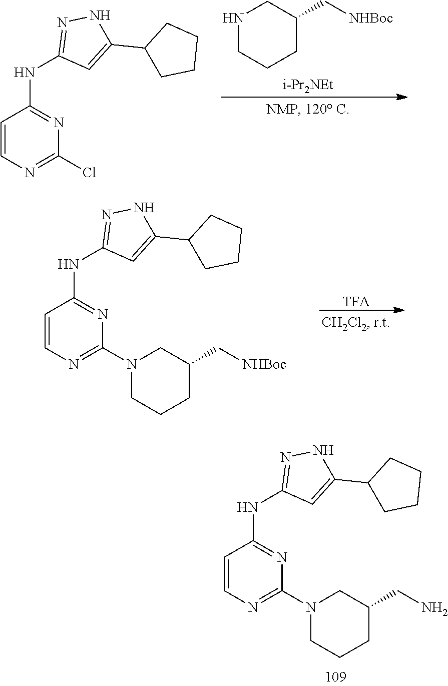

### tert-Butyl N-[[(3S)-1-[4-[(5-Cyclopentyl-1H-pyrazol-3-yl)amino]pyrimidin-2-yl]-3-piperidyl]methyl]carbamate

A solution of 2-chloro-N-(5-cyclopentyl-1H-pyrazol-3-yl)pyrimidin-4-amine (205 mg, 0.777 mmol), N,N-diisopropylethylamine (0.41 mL, 2.3 mmol), and tert-butyl N-[[(3R)-3-piperidyl]methyl]carbamate (333 mg, 1.55 mmol) in n-butanol (5 mL) was heated to 100° C. and stirred for 72 hours. The reaction mixture was then concentrated to dryness under reduced pressure. The residue was suspended in water (30 mL), then extracted with CH₂Cl₂ (3×20 mL). The combined organic extracts were dried over MgSO4, then concentrated to dryness under reduced pressure to afford the title compound as an oil (362 mg, 0.771 mmol, 99%).

¹H NMR (400 MHz, DMSO-d₆) δ 11.90 (s, 1H), 9.32 (s, 1H), 7.84 (d, J=5.6 Hz, 1H), 6.86 (t, J=5.8 Hz, 1H), 6.30-6.08 (m, 2H), 4.54-4.45 (m, 2H), 2.90-2.77 (m, 3H), 2.08-1.90 (m, 2H), 1.84-1.56 (m, 9H), 1.44-1.30 (m, 12H).

UPLC-MS (basic method, 4 min): Rt: 1.97 min, m/z: 442.3 [M+H]⁺.

### 2-[(3S)-3-(Aminomethyl)-1-piperidyl]-N-(5-cyclopentyl-1H-pyrazol-3-yl)pyrimidin-4-amine

To a solution of tert-butyl N-[[(3S)-1-[4-[(5-cyclopropyl-1H-pyrazol-3-yl)amino]pyrimidin-2-yl]-3-piperidyl]methyl]carbamate (360 mg, 0.815 mmol) in CH₂Cl₂ (5 mL) was added trifluoracetic acid (5.0 mL, 66 mmol) and the reaction mixture was stirred at ambient temperature for 1 hour, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase basic prepHPLC, to afford the title compound as a solid (128 mg, 0.375 mmol, 46%).

¹H NMR (400 MHz, DMSO-d₆) δ 11.91 (s, 1H), 9.34 (s, 1H), 7.84 (d, J=5.7 Hz, 1H), 6.25 (s, 2H), 4.51 (d, J=12.9 Hz, 1H), 4.42 (d, J=12.9 Hz, 1H), 3.05-2.83 (m, 2H), 2.67 (dd, J=13.0, 10.2 Hz, 1H), 2.45 (dd, J=6.6, 3.8 Hz, 2H), 2.02-1.92 (m, 2H), 1.87-1.78 (m, 1H), 1.77-1.49 (m, 8H), 1.39 (tt, J=12.0, 7.4 Hz, 2H).

UPLC-MS (basic method, 4 min): rt=1.44 min, m/z=342.2 [M+H]⁺.

### Example 84: Synthesis of Compound 110

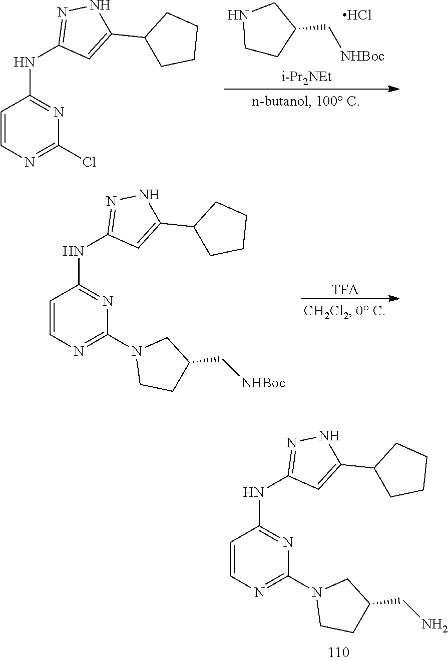

### tert-Butyl N-[[(3S)-1-[4-[(5-Cyclopentyl-1H-pyrazol-3-yl)amino]pyrimidin-2-yl]pyrrolidin-3-yl]methyl]carbamate

A solution of 2-chloro-N-(5-cyclopentyl-1H-pyrazol-3-yl)pyrimidin-4-amine (300 mg, 1.14 mmol), tert-butyl N-[(3R)-pyrrolidin-3-ylmethyl]carbamate hydrochloride (404 mg, 1.71 mmol) and N,N-diisopropylethylamine (0.79 mL, 4.6 mmol) in n-butanol (6 mL) was heated to 100° C. and stirred for 24 hours. The reaction mixture was then concentrated to dryness under reduced pressure. The residue was dissolved in CH₂Cl₂ (30 mL), then the organic layer was washed with water (2×10 mL) then a saturated aqueous brine solution (30 mL). The organic layer was dried over MgSO₄, then concentrated to dryness under reduced pressure to afford the title compound as an oil (372 mg, 0.801 mmol, 70%).

¹H NMR (400 MHz, DMSO-d₆) δ 11.91 (s, 1H), 9.46 (s, 1H), 7.83 (d, J=5.8 Hz, 1H), 7.01 (t, J=5.8 Hz, 1H), 6.40 (s, 1H), 6.22 (s, 1H), 3.65-3.53 (m, 2H), 3.17 (d, J=5.2 Hz, 1H), 3.04-2.96 (m, 3H), 2.43-2.32 (m, 1H), 2.01-1.88 (m, 3H), 1.76-1.52 (m, 7H), 1.42-1.35 (m, 10H).

UPLC-MS (basic method, 4 min): Rt: 1.91 min, m/z: 428.4 [M+H]⁺.

### 2-[(3S)-3-(Aminomethyl)pyrrolidin-1-yl]-N-(5-cyclopentyl-1H-pyrazol-3-yl)pyrimidin-4-amine

To a solution of tert-butyl (S)-(1-(4-((5-cyclopentyl-1H-pyrazol-3-yl)amino)pyrimidin-2-yl)pyrrolidin-3-yl)(methyl)carbamate (372 mg, 0.870 mmol) in CH₂Cl₂ (5 mL) was added trifluoracetic acid (5.0 mL, 69 mmol) at 0° C. The reaction mixture was held at 0° C. and stirred for 30 minutes, after which the reaction mixture was concentrated to dryness under reduced pressure. The crude residue was loaded onto an SCX-2 cartridge, the cartridge was then washed with methanol (20 mL), then the compound was eluted with 2 M NH₃ in methanol (20 mL), and the eluent was concentrated to dryness under reduced pressure. The crude residue was purified by reversed-phase basic prepHPLC, to afford the title compound as a solid (45 mg, 129 mmol, 16%).

¹H NMR (400 MHz, DMSO-d₆) δ 11.85 (s, 1H), 9.33 (s, 1H), 7.81 (d, J=5.7 Hz, 1H), 6.38 (s, 1H), 6.17 (s, 1H), 3.64-3.50 (m, 2H), 3.18-3.10 (m, 1H), 3.06-2.88 (m, 1H), 2.64-2.54 (m, 2H), 2.29-2.14 (m, 1H), 2.05-1.88 (m, 4H), 1.76-1.39 (m, 9H).

UPLC-MS (basic method, 4 min): Rt: 1.46 min, m/z: 328.3 [M+H]⁺.

### Example 85: Synthesis of Compound 111

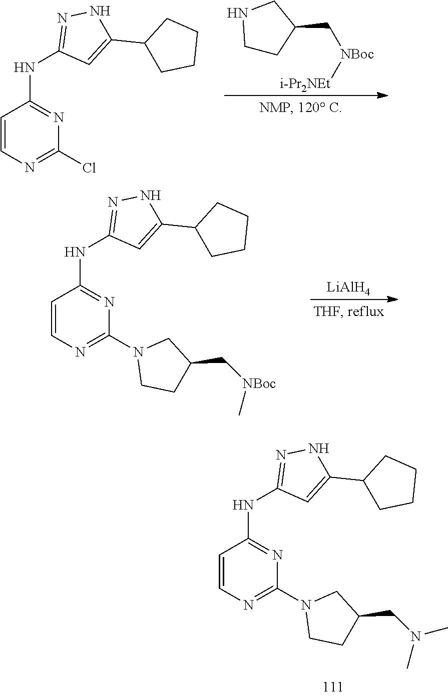

### 2-Chloro-N-(5-cyclopentyl-1H-pyrazol-3-yl)pyrimidin-4-amine

A solution of 5-cyclopentyl-1H-pyrazol-3-amine (5.00 g, 33.1 mmol), 2,4-dichloropyrimidine (5.42 g, 36.4 mmol) and N,N-diisopropylethylamine (9.5 mL, 55 mmol) in DMSO (50 mL) was heated to 60° C. and stirred for 18 hours. The reaction mixture was then allowed to cool to room temperature and then poured into ice water (ca. 2 L) resulting in a precipitation. The precipitate was collected by filtration and the filter cake washed with water (100 mL) followed by CH₂Cl₂ (100 mL). The filter cake was dried under reduced pressure to afford the title compound as a solid (7.22 g, 27.4 mmol, 75%).

¹H NMR (400 MHz, DMSO-d₆) δ 12.18 (s, 1H), 10.29 (s, 1H), 8.16 (s, 1H), 7.26 (s, 1H), 6.07 (s, 1H), 3.02 (p, J=8.1 Hz, 1H), 2.07-1.95 (m, 2H), 1.81-1.50 (m, 6H).

UPLC-MS (basic method, 2 min): Rt: 1.04 min, m/z: 264.0 [M+H]⁺.

### tert-Butyl N-[[(3S)-1-[4-[(5-Cyclopentyl-1H-pyrazol-3-yl)amino]pyrimidin-2-yl]pyrrolidin-3-yl]methyl]-N-methyl-carbamate

A solution of 2-chloro-N-(5-cyclopentyl-1H-pyrazol-3-yl)pyrimidin-4-amine (250 mg, 0.948 mmol), tert-butyl (S)-methyl(pyrrolidin-3-ylmethyl)carbamate (244 mg, 1.14 mmol) and N,N-diisopropylethylamine (0.41 mL, 2.4 mmol) in NMP (2.5 mL) was heated to 120° C. and stirred for 20 hours in a sealed tube. The reaction mixture was then added dropwise to ice water (ca. 100 mL) resulting in the formation of a precipitate. The precipitate was collected by filtration, then washed with water (2×10 mL). The material was then dried under reduced pressure for 1 hour to afford the title compound as a solid (324 mg, 0.734 mmol, 77%).

¹H NMR (400 MHz, DMSO-d₆) δ 11.86 (s, 1H), 9.35 (s, 1H), 7.82 (d, J=5.7 Hz, 1H), 6.42 (s, 1H), 6.18 (s, 1H), 3.63-3.38 (m, 3H), 3.29-3.09 (m, 3H), 3.00 (p, J=8.1 Hz, 1H), 2.81 (s, 3H), 2.59-2.52 (m, 1H), 2.06-1.88 (m, 3H), 1.76-1.50 (m, 7H), 1.46-1.24 (m, 9H).

UPLC-MS (basic method, 2 min): Rt: 1.26 min, m/z: 442.2 [M+H]⁺.

### N-(5-Cyclopentyl-1H-pyrazol-3-yl)-2-[(3R)-3-[(dimethylamino)methyl]pyrrolidin-1-yl]pyrimidin-4-amine

To a solution of tert-butyl N-[[(3S)-1-[4-[(5-Cyclopentyl-1H-pyrazol-3-yl)amino]pyrimidin-2-yl]pyrrolidin-3-yl]methyl]-N-methyl-carbamate (150 mg, 0.340 mmol) in anhydrous THE (10 mL) was added a 2.4 M solution of lithium aluminum hydride in THE (1.4 mL, 3.4 mmol) at 0° C. The reaction mixture was then heated to reflux and stirred for 6 hours. The reaction was then cooled to 0° C., then quenched by dropwise addition of water (140 μL), followed by a 15% w/v aqueous solution of NaOH (140 μL), followed by water (420 μL). The reaction mixture was warmed to room temperature and stirred for 15 minutes. MgSO₄ (ca. 1 g) was then added, and the reaction mixture was stirred for 16 hours. The reaction mixture was filtered through a pad of celite, then the filter cake was washed with CH₂Cl₂ (20 mL), followed by EtOH (20 mL), then the filtrate was concentrated to dryness under reduced pressure. The crude residue was dissolved in MeOH (2 mL), then loaded onto a SCX-2 cartridge. The cartridge was washed with MeOH (20 mL), then the compound was eluted with a 2 M solution of NH₃ in MeOH (20 mL). The eluent was concentrated to dryness under reduced pressure to afford the title compound as a solid (95 mg, 0.27 mmol, 79%).

¹H NMR (400 MHz, DMSO-d₆) δ 11.86 (s, 1H), 9.34 (s, 1H), 7.82 (d, J=5.6 Hz, 1H), 6.43 (s, 1H), 6.18 (s, 1H), 3.71-3.62 (m, 1H), 3.62-3.51 (m, 1H), 3.45-3.36 (m, 1H), 3.23-3.10 (m, 1H), 3.00 (p, J=7.9 Hz, 1H), 2.43 (p, J=7.3 Hz, 1H), 2.24 (d, J=7.5 Hz, 2H), 2.16 (s, 6H), 2.07-1.92 (m, 3H), 1.75-1.51 (m, 7H).

UPLC-MS (basic method, 4 min): Rt: 1.62 min, m/z: 356.4 [M+H]⁺.

### Example 86: Synthesis of Compound 112

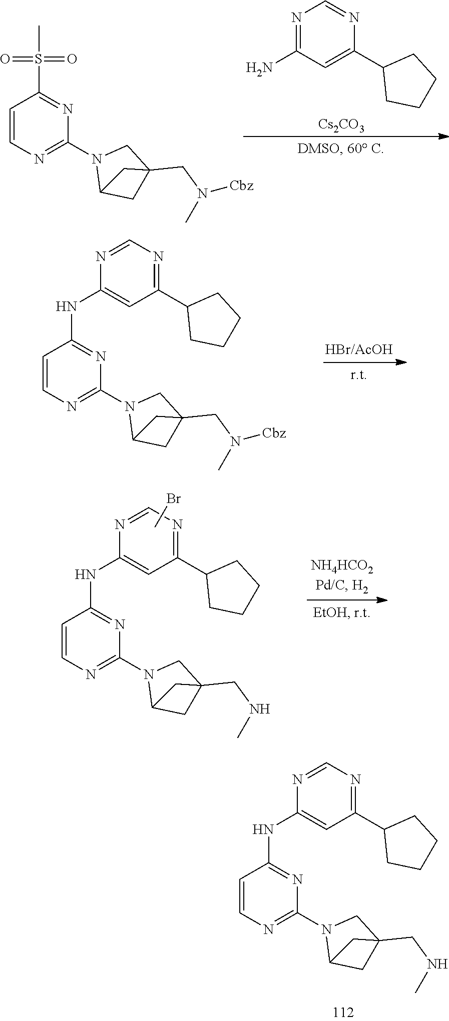

### Benzyl N-[[2-[4-[(6-Cyclopentylpyrimidin-4-yl)amino]pyrimidin-2-yl]-2-azabicyclo[2.1.1]hexan-4-yl]methyl]-N-methyl-carbamate

A solution of benzyl N-methyl-N-[[2-(4-methylsulfonylpyrimidin-2-yl)-2-azabicyclo[2.1.1]hexan-4-yl]methyl]carbamate (150 mg, 0.360 mmol), 6-cyclopentylpyrimidin-4-amine (88 mg, 0.54 mmol) and cesium carbonate (352 mg, 1.08 mmol) in DMSO (4 mL) was heated to 60° C. in a sealed tube and stirred for 18 hours. The reaction mixture was diluted with CH₂Cl₂ (15 mL), and the organic phase was washed with a saturated aqueous NaHCO₃ solution (15 mL) then a saturated aqueous brine solution (15 mL). The organic layer was separated, dried over Na₂SO₄ and concentrated to dryness under reduced pressure. The crude residue was purified by normal-phase flash column chromatography, over silica gel, eluting with a gradient of CH₂Cl₂ to 1:9 2 M NH₃ in MeOH:CH₂Cl₂ to afford the title compound as an oil (172 mg, 0.340 mmol, 96%).

¹H NMR (400 MHz, DMSO-d₆) δ 10.15 (s, 1H), 8.66 (d, J=1.2 Hz, 1H), 8.07 (d, J=5.6 Hz, 1H), 7.38-7.27 (m, 5H), 6.63 (s, 1H), 5.11-5.04 (m, 2H), 4.77-4.69 (m, 1H), 3.60 (s, 2H), 3.38 (s, 2H), 3.09-3.04 (m, 1H), 2.97-2.90 (m, 3H), 2.03-1.95 (m, 2H), 1.89-1.60 (m, 8H), 1.50-1.41 (m, 2H).

UPLC-MS (basic method, 2 min): Rt: 1.22 mins, m/z=500.3 [M+H]⁺.

### Bromo-6-cyclopentyl-N-[2-[4-(methylaminomethyl)-2-azabicyclo[2.1.1]hexan-2-yl]pyrimidin-4-yl]pyrimidin-4-amine

To benzyl N-[[2-[4-[(6-cyclopentylpyrimidin-4-yl)amino]pyrimidin-2-yl]-2-azabicyclo[2.1.1]hexan-4-yl]methyl]-N-methyl-carbamate (172 mg, 0.344 mmol) was added a 30% solution of HBr in acetic acid (3 mL) and the reaction mixture stirred at room temperature for 2 hours. The reaction mixture was concentrated to dryness, then dissolved in MeOH (2 mL) and loaded onto an SCX-2 cartridge. The cartridge was washed with MeOH (25 mL), then the compound was eluted with 2 M NH₃ in MeOH (25 mL). The eluent was concentrated to dryness under reduced pressure. The residue was further purified by reversed-phase basic prepHPLC to afford the title compound as a solid (26 mg, 80 μmol, 31%).

¹H NMR (400 MHz, DMSO-d₆) δ 8.77 (d, J=1.3 Hz, 1H), 8.33 (s, 1H), 6.91 (s, 1H), 4.76-4.71 (m, 1H), 3.51-3.44 (m, 3H), 2.75-2.70 (m, 2H), 2.60 (s, 4H), 2.35-2.30 (m, 1H), 2.11-1.97 (m, 6H), 1.92-1.87 (m, 1H), 1.58-1.54 (m, 3H).

UPLC-MS (basic method, 6 min): Rt: 3.62 mins, m/z=442.2 [M+H]⁺.

### N-(6-Cyclopentylpyrimidin-4-yl)-2-[4-(methylaminomethyl)-2-azabicyclo[2.1.1]hexan-2-yl]pyrimidin-4-amine

A solution of 2-bromo-6-cyclopentyl-N-[2-[4-(methylaminomethyl)-2-azabicyclo[2.1.1]hexan-2-yl]pyrimidin-4-yl]pyrimidin-4-amine (101 mg, 0.227 mmol), ammonium formate (143 mg, 2.27 mmol) and palladium on activated carbon (24 mg, 23 μmol) in ethanol (3 mL) was placed under an atmosphere of hydrogen and left to stir at room temperature for 72 hours. The reaction mixture was filtered through celite, the filter cake was washed with EtOAc (10 mL) and concentrated to dryness under reduced pressure. The crude residue was purified by basic reversed-phase prepHPLC to give the title compound as a semi-solid (19 mg, 50 μmol, 23%).

¹H NMR (400 MHz, DMSO-d₆) δ 10.14 (s, 1H), 8.65 (d, J=1.2 Hz, 1H), 8.06 (d, J=5.6 Hz, 1H), 6.56 (s, 1H), 4.74 (d, J=1.7 Hz, 1H), 3.41 (s, 2H), 3.12-3.00 (m, 1H), 2.77 (s, 2H), 2.32 (s, 3H), 2.10-1.97 (m, 2H), 1.88-1.80 (m, 2H), 1.80-1.61 (m, 6H), 1.48-1.38 (m, 2H).

UPLC-MS (basic method, 4 min): Rt: 1.48 mins, m/z=366.2 [M+H⁺].

### Example 87: Synthesis of Compound 113

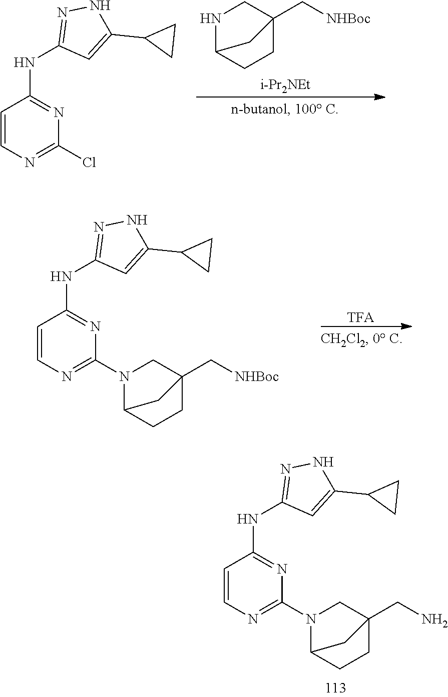

### tert-Butyl N-[[2-[4-[(5-Cyclopropyl-1H-pyrazol-3-yl)amino]pyrimidin-2-yl]-2-azabicyclo[2.2.1]heptan-4-yl]methyl]carbamate

To a solution of 2-chloro-N-(5-cyclopropyl-1H-pyrazol-3-yl)pyrimidin-4-amine (265 mg, 1.12 mmol) and tert-butyl N-({2-azabicyclo[2.2.1]heptan-4-yl}methyl)carbamate (293 mg, 1.29 mmol) in n-butanol (13 mL) was added N,N-diisopropylethylamine (0.59 mL, 3.4 mmol). The reaction mixture was heated to 100° C. and stirred for 18 h. The reaction mixture was concentrated under reduced pressure and diluted with water (30 mL). The resulting solution was extracted with DCM (3×20 mL). The combined organic fractions were washed with a saturated brine solution (20 mL), dried over Na₂SO₄ and concentrated to dryness under reduced pressure to afford the title compound as an oil (479 mg, 0.877 mmol, 78%).

¹H NMR (400 MHz, DMSO-d₆) δ 11.83 (s, 1H), 9.26 (s, 1H), 7.78 (d, J=5.6 Hz, 1H), 7.01 (s, 1H), 6.18 (s, 2H), 4.46 (s, 1H), 3.28 (d, J=5.4 Hz, 2H), 3.20 (d, J=6.2 Hz, 2H), 3.07 (s, 1H), 1.85 (s, 1H), 1.70 (s, 1H), 1.52 (d, J=10.5 Hz, 2H), 1.39 (s, 9H), 1.36 (s, 2H), 0.90 (s, 2H), 0.78-0.56 (m, 2H).

UPLC-MS (basic method, 4 min): rt=1.64 min, m z: 426.4 [M+H]⁺.

### 2-[4-(Aminomethyl)-2-azabicyclo[2.2.1]heptan-2-yl]-N-(5-cyclopropyl-1H-pyrazol-3-yl)pyrimidin-4-amine

To a solution tert-butyl N-[[2-[4-[(5-cyclopropyl-1H-pyrazol-3-yl)amino]pyrimidin-2-yl]-2-azabicyclo[2.2.1]heptan-4-yl]methyl]carbamate (74 mg, 0.17 mmol) in CH₂Cl₂ (1.9 mL) was added trifluoroacetic acid (4.0 mL, 53 mmol) at 0° C. The reaction mixture was warmed to room temperature and stirred for 30 minutes. The reaction mixture was then concentrated to dryness under reduced pressure. The residue was then loaded onto a SCX-2 cartridge. The cartridge was washed with MeOH (25 mL) then the compound was eluted with 2 M solution of NH₃ in MeOH (25 mL). The eluent was concentrated to dryness under reduced pressure. The product was purified by reversed-phase basic prepHPLC. The residue was then loaded onto an SCX-2 cartridge. The cartridge was washed with MeOH (25 mL) then the compound was eluted with 2 M solution of NH₃ in MeOH (25 mL). The eluent was concentrated to dryness under reduced pressure to afford the title compound as a solid (30 mg, 88 μmol, 50%).

¹H NMR (400 MHz, DMSO-d₆) δ 11.87 (s, 1H), 9.27 (s, 1H), 7.79 (d, J=5.7 Hz, 1H), 6.17 (s, 2H), 4.49 (s, 1H), 3.14 (d, J=9.6 Hz, 1H), 2.92 (s, 2H), 1.85 (td, J=8.5, 4.3 Hz, 1H), 1.66-1.53 (m, 3H), 1.39 (d, J=14.9 Hz, 2H), 0.90 (d, J=8.2 Hz, 3H), 0.70-0.59 (m, 3H).

UPLC-MS (basic method, 4 mins): Rt: 1.68 min, m/z=326.3 [M+H]⁺.

### Example 88: Synthesis of Compound 114

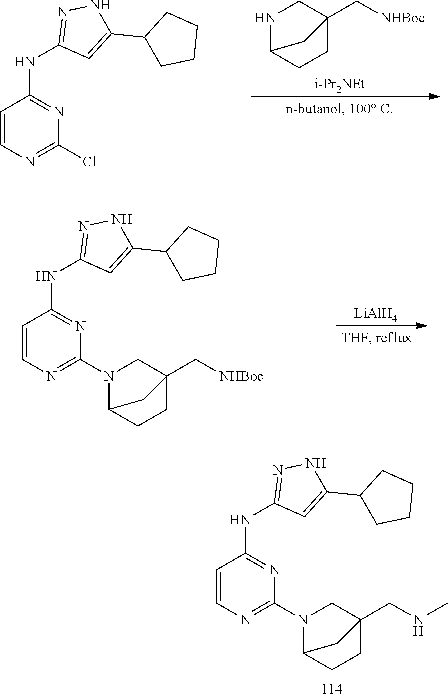 [SMILES: "i-Pr2NEt" ; Clc1nccc(Nc2cc(C3CCCC3)[nH]n2)n1 ; CC(C)(C)OC(=O)NCC12CCC(C1)NC2 ; CC(C)(C)OC(=O)NCC12CCC(C1)N(c1nccc(Nc3cc(C4CCCC4)[nH]n3)n1)C2 ; "LiAlH4" ; CNCC12CCC(C1)N(c1nccc(Nc3cc(C4CCCC4)[nH]n3)n1)C2]

### tert-Butyl N-[[2-[4-[(5-Cyclopentyl-1H-pyrazol-3-yl)amino]pyrimidin-2-yl]-2-azabicyclo[2.2.1]heptan-4-yl]methyl]carbamate

A solution of 2-chloro-N-(5-cyclopentyl-1H-pyrazol-3-yl)pyrimidin-4-amine (202 mg, 0.764 mmol), tert-butyl N-({2-azabicyclo[2.2.1]heptan-4-yl}methyl)carbamate (190 mg, 0.840 mmol), and N,N-diisopropylethylamine (0.20 mL, 1.2 mmol) in n-butanol (5 mL) was heated to 100° C. and stirred for 24 hours. The reaction mixture was then concentrated to dryness under reduced pressure. The residue was then suspended in water (20 mL) then extracted with CH₂Cl₂ (3×20 mL), dried over MgSO₄, then concentrated under reduced pressure. The crude material was purified by normal-phase column chromatography, over silica gel, eluting with a gradient of CH₂Cl₂ to 1:4 2 M NH₃ MeOH:CH₂Cl₂ to afford the title compound as an oil (232 mg, 0.486 mmol, 64%).

¹H NMR (400 MHz, DMSO-d₆) δ 11.86 (s, 1H), 9.31 (s, 1H), 7.79 (d, J=5.8 Hz, 1H), 7.00 (s, 1H), 6.34 (s, 1H), 6.19 (s, 2H), 4.48 (s, 1H), 3.30-3.18 (m, 3H), 3.13-2.96 (m, 2H), 1.98 (s, 2H), 1.77-1.50 (m, 10H), 1.45-1.28 (m, 11H).

UPLC-MS (basic method, 4 min): Rt: 1.87 min, m/z=454.4 [M+H]⁺.

### 2-[4-(Aminomethyl)-2-azabicyclo[2.2.1]heptan-2-yl]-N-(5-cyclopropyl-1H-pyrazol-3-yl)pyrimidin-4-amine

To a solution of tert-butyl N-[[2-[4-[(5-cyclopentyl-1H-pyrazol-3-yl)amino]pyrimidin-2-yl]-2-azabicyclo[2.2.1]heptan-4-yl]methyl]carbamate (108 mg, 0.226 mmol) in anhydrous THF (7 mL) was added a 2.4 M solution of lithium aluminum hydride in THE (0.94 mL, 2.3 mmol) at 0° C. The reaction mixture was then heated to reflux and stirred for 3 hours. The reaction was then cooled to 0° C., then quenched by dropwise addition of water (900 μL), followed by a 15% w/v aqueous solution of NaOH (900 μL), followed by water (2.7 mL). The reaction mixture was warmed to room temperature and stirred for 15 minutes. MgSO₄ (ca. 1 g) was then added, and the reaction mixture was stirred for 16 hours. The reaction mixture was filtered through a pad of celite, then the filter cake was washed with EtOAc (20 mL), then the filtrate was concentrated to dryness under reduced pressure. The crude residue was purified by reversed-phase basic prepHIPLC to afford the title compound as a solid (12 mg, 33 μmol, 14%).

¹H NMR (400 MHz, DMSO-d₆) δ 11.84 (s, 1H), 9.30 (s, 1H), 7.79 (d, J=5.7 Hz, 1H), 6.34 (s, 1H), 6.14 (s, 1H), 4.48 (s, 1H), 3.17 (d, J=11.9 Hz, 1H), 3.00 (q, J=7.8 Hz, 1H), 2.68 (d, J=12.9 Hz, 2H), 2.33 (s, 3H), 1.96 (d, J=10.0 Hz, 2H), 1.73-1.50 (m, 11H), 1.39 (s, 2H).

UPLC-MS (basic method, 4 min): Rt: 1.46 min, m/z=368.4 [M+H]⁺.

### Example 89: Synthesis of Compound 115

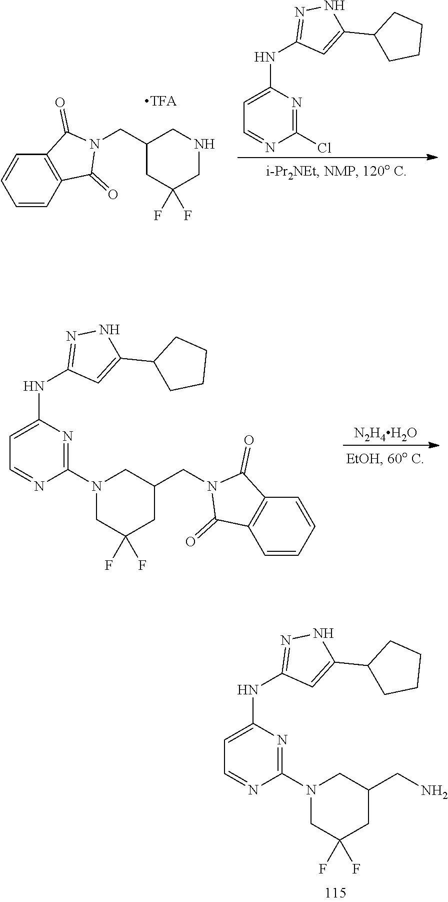

### 2-[[1-[4-[(5-Cyclopentyl-1H-pyrazol-3-yl)amino]pyrimidin-2-yl]-5,5-difluoro-3-piperidyl]methyl]isoindoline-1,3-dione

A solution of 2-chloro-N-(5-cyclopentyl-1H-pyrazol-3-yl)pyrimidin-4-amine (200 mg, 0.758 mmol), 2-[(5,5-difluoro-3-piperidyl)methyl]isoindoline-1,3-dione;2,2,2-trifluoroacetic acid (359 mg, 0.910 mmol) and N,N-diisopropylethylamine (0.46 mL, 2.65 mmol) in anhydrous NMP (3 mL) was heated to 120° C. and stirred for 16 hours. The reaction mixture was then cooled to room temperature, then added dropwise to stirring ice water (ca. 50 mL) resulting in the formation of a precipitate. The precipitate was collected by filtration, the filter cake was washed with water (2×10 mL), then dried under reduced pressure. The crude material was purified by normal-phase column chromatography, over silica gel, eluting with a gradient of CH₂Cl₂ to 9:1 CH₂Cl₂:2 M NH₃ in MeOH to afford the title compound as a solid (64 mg, 0.13 mmol, 17%).

¹H NMR (400 MHz, DMSO-d₆) δ 11.95 (s, 1H), 9.48 (s, 1H), 7.93-7.81 (m, 5H), 6.47-6.02 (m, 2H), 4.83 (t, J=12.1 Hz, 1H), 4.65 (d, J=13.2 Hz, 1H), 3.65-3.64 (m, 2H), 3.25-3.14 (m, 1H), 3.01 (d, J=7.8 Hz, 1H), 2.82 (t, J=12.1 Hz, 1H), 2.29-2.08 (m, 2H), 2.05-1.78 (m, 3H), 1.79-1.49 (m, 6H).

¹⁹F NMR (376 MHz, DMSO-d₆) δ −97.7-−98.8 (m), −101.6-−102.7 (m).

UPLC-MS (basic method, 2 min): Rt: 1.17 min, m/z=508.2 [M+H]⁺.

### 2-[5-(Aminomethyl)-3,3-difluoro-1-piperidyl]-N-(5-cyclopentyl-1H-pyrazol-3-yl)pyrimidin-4-amine

To a solution of 2-[[1-[4-[(5-cyclopentyl-1H-pyrazol-3-yl)amino]pyrimidin-2-yl]-5,5-difluoro-3-piperidyl]methyl]isoindoline-1,3-dione (60 mg, 0.12 mmol) in ethanol (5 mL) was added hydrazine hydrate (59 μL, 0.59 mmol). The reaction mixture was then heated to 60° C. and stirred for 5 hours. The reaction mixture was then filtered through celite, and the filtrate was concentrated to dryness under reduced pressure. The crude residue was dissolved in MeOH, then loaded onto a SCX-2 cartridge. The cartridge was washed with MeOH (20 mL), then the compound was eluted with 2 M NH₃ in MeOH (20 mL). The eluent was concentrated to dryness under reduced pressure to afford the title compound as a solid (37 mg, 98 μmol, 83%).

¹H NMR (400 MHz, Chloroform-d) δ 8.00 (d, J=5.7 Hz, 1H), 7.03 (s, 1H), 6.21 (d, J=5.7 Hz, 1H), 6.05 (s, 1H), 4.62-4.44 (m, 1H), 4.43-4.29 (m, 1H), 3.72-3.50 (m, 1H), 3.29-3.13 (m, 1H), 3.03 (p, J=7.9 Hz, 1H), 2.83-2.61 (m, 2H), 2.34-2.18 (m, 1H), 2.14-1.92 (m, 3H), 1.86-1.56 (m, 7H).

¹⁹F NMR (376 MHz, Chloroform-d) δ-δ −98.0-−99.2 (m), −100.8-−102.2 (m).

UPLC-MS (basic method, 4 min): Rt: 1.46 min, m/z=378.4 [M+H]⁺.

### Example 90: Synthesis of Compound 116

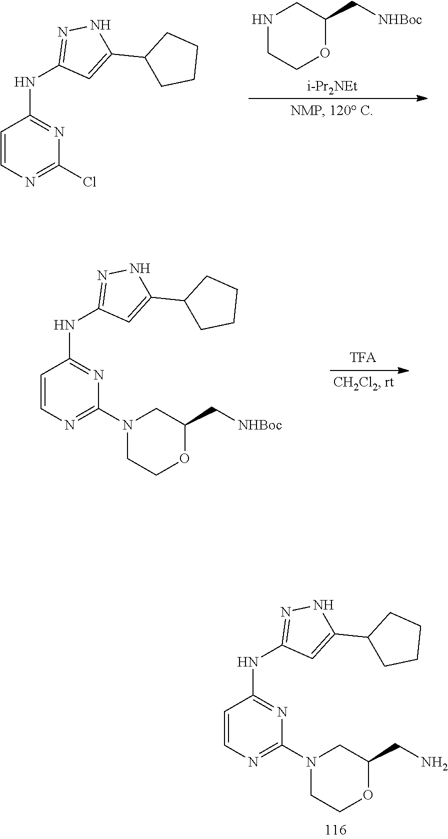 [SMILES: "i-Pr2NEt" ; Clc1nccc(Nc2cc(C3CCCC3)[nH]n2)n1 ; CC(C)(C)OC(=O)NC[C@@H]1CNCCO1 ; CC(C)(C)OC(=O)NC[C@@H]1CN(c2nccc(Nc3cc(C4CCCC4)[nH]n3)n2)CCO1 ; NC[C@@H]1CN(c2nccc(Nc3cc(C4CCCC4)[nH]n3)n2)CCO1]

### tert-Butyl N-[[(2R)-4-[4-[(5-Cyclopentyl-1H-pyrazol-3-yl)amino]pyrimidin-2-yl]morpholin-2-yl]methyl]carbamate

A solution of 2-chloro-N-(5-cyclopropyl-1H-pyrazol-3-yl)pyrimidin-4-amine (277 mg, 1.05 mmol), (S)-tert-butyl (morpholin-2-ylmethyl)carbamate (250 mg, 1.16 mmol) and N,N-diisopropylethylamine (605 μL, 3.47 mmol) in NMP (4.5 mL) was heated to 120° C. and stirred for 16 hours. The reaction mixture was cooled to room temperature, then added dropwise to ice water (ca. 100 mL) resulting in the formation of a precipitate. The precipitate was collected by filtration, dissolved in EtOAc, dried over Na₂SO₄, then concentrated to dryness under reduced pressure to afford the title compound as a solid (403 mg, 0.799 mmol, 69%).

¹H NMR (400 MHz, DMSO-d₆) δ 11.93 (s, 1H), 9.41 (s, 1H), 7.89 (d, J=5.7 Hz, 1H), 6.94-6.87 (m, 1H), 6.34 (s, 1H), 6.18 (s, 1H), 4.45 (d, J=13.0 Hz, 1H), 4.33 (d, J=13.0 Hz, 1H), 3.93-3.85 (m, 1H), 3.50-3.43 (m, 2H), 3.14-2.97 (m, 3H), 3.97-2.84 (m, 1H), 2.64-2.54 (m, 1H), 2.03-1.93 (m, 2H), 1.73-1.68 (m, 2H), 1.63-1.54 (m, 4H), 1.38 (s, 9H).

UPLC-MS (basic method, 4 min): Rt: 1.80 min, m/z=444.4 [M+H]⁺.

### 2-[(2R)-2-(Aminomethyl)morpholin-4-yl]-N-(5-cyclopentyl-1H-pyrazol-3-yl)pyrimidin-4-amine

To a solution of tert-butyl N-[[(2R)-4-[4-[(5-cyclopentyl-1H-pyrazol-3-yl)amino]pyrimidin-2-yl]morpholin-2-yl]methyl]carbamate (140 mg, 0.315 mmol) in CH₂Cl₂ (2.1 mL) was added trifluoroacetic acid (483 μl, 6.31 mmol) then the reaction mixture was stirred at room temperature for 16 hours. The reaction mixture was concentrated to dryness under reduced pressure. The residue was loaded onto a SCX-2 cartridge, the cartridge was washed with MeOH (25 mL), then the compound was eluted with 2 M NH₃ in MeOH (25 mL). The eluent was concentrated to dryness under reduced pressure. The crude material was purified by normal-phase column chromatography, over silica gel, eluting with a gradient of CH₂Cl₂ to 1:4 MeOH:CH₂Cl₂ to afford the title compound as a solid (54 mg, 0.16 mmol, 49%).

¹H NMR (400 MHz, DMSO-d₆) δ 11.93 (s, 1H), 9.42 (s, 1H), 7.88 (d, J=5.9 Hz, 1H), 6.28 (s, 1H), 6.21 (s, 1H), 4.54-4.45 (m, 1H), 4.39-4.31 (m, 1H), 3.95-3.86 (m, 1H), 3.52-3.41 (m, 1H), 3.06-2.94 (m, 1H), 2.94-2.83 (m, 1H), 2.71-2.55 (m, 3H), 2.05-1.92 (m, 2H), 1.76-1.49 (m, 7H).

UPLC-MS (basic method, 4 min): Rt: 1.28 min, m/z=344.3 [M+H]⁺.

### Example 91: Synthesis of Compound 117

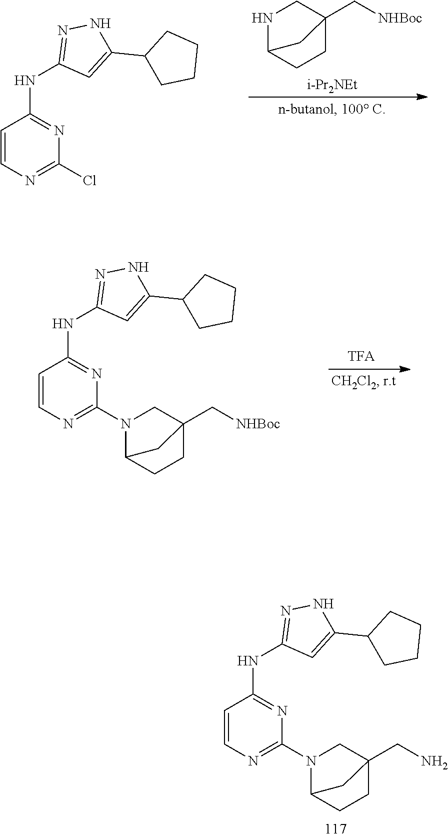 [SMILES: "i-Pr2NEt" ; Clc1nccc(Nc2cc(C3CCCC3)[nH]n2)n1 ; CC(C)(C)OC(=O)NCC12CCC(C1)NC2 ; CC(C)(C)OC(=O)NCC12CCC(C1)N(c1nccc(Nc3cc(C4CCCC4)[nH]n3)n1)C2 ; NCC12CCC(C1)N(c1nccc(Nc3cc(C4CCCC4)[nH]n3)n1)C2]

### 2-[4-(Aminomethyl)-2-azabicyclo[2.2.1]heptan-2-yl]-N-(5-cyclopropyl-1H-pyrazol-3-yl)pyrimidin-4-amine

To a solution tert-butyl N-[[2-[4-[(5-cyclopropyl-1H-pyrazol-3-yl)amino]pyrimidin-2-yl]-2-azabicyclo[2.2.1]heptan-4-yl]methyl]carbamate (74 mg, 0.17 mmol) in CH₂Cl₂ (1.9 mL) was added trifluoroacetic acid (4.0 mL, 52 mmol) at 0° C. The reaction mixture was warmed to room temperature, then stirred for 30 minutes. The reaction mixture was then concentrated to dryness under reduced pressure. The residue was then loaded onto a SCX-2 cartridge. The cartridge was washed with MeOH (25 mL) then the compound was eluted with a 2 M solution of NH₃ in MeOH (25 mL). The eluent was concentrated to dryness under reduced pressure. The residue was purified by reversed-phase basic prepHPLC to afford the title compound as a solid (30 mg, 88 μmol, 50%).

¹H NMR (400 MHz, DMSO-d₆) δ 11.85 (s, 1H), 9.30 (s, 1H), 7.79 (d, J=5.7 Hz, 1H), 6.48-6.00 (m, 2H), 4.49 (s, 1H), 3.11 (s, 1H), 3.00 (p, J=7.7 Hz, 1H), 2.79 (s, 2H), 1.97 (d, J=9.7 Hz, 2H), 1.78-1.47 (m, 11H), 1.42-1.20 (m, 2H).

UPLC-MS (basic method, 4 min): Rt: 1.36, m/z=354.4 [M+H]⁺.

### Example 92: Synthesis of Compound 118

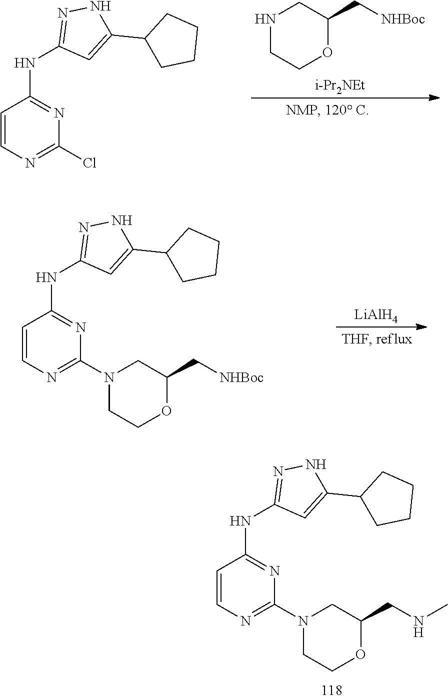 [SMILES: "i-Pr2NEt" ; Clc1nccc(Nc2cc(C3CCCC3)[nH]n2)n1 ; CC(C)(C)OC(=O)NC[C@@H]1CNCCO1 ; CC(C)(C)OC(=O)NC[C@@H]1CN(c2nccc(Nc3cc(C4CCCC4)[nH]n3)n2)CCO1 ; "LiAlH4" ; CNC[C@@H]1CN(c2nccc(Nc3cc(C4CCCC4)[nH]n3)n2)CCO1]

### N-(5-Cyclopentyl-1H-pyrazol-3-yl)-2-[(2R)-2-(methylaminomethyl)morpholin-4-yl]pyrimidin-4-amine

To a solution tert-butyl N-[[(2R)-4-[4-[(5-cyclopentyl-1H-pyrazol-3-yl)amino]pyrimidin-2-yl]morpholin-2-yl]methyl]carbamate (179 mg, 0.403 mmol) in anhydrous THF (8.0 mL) was added a 2.4 M solution of lithium aluminum hydride in THF (1.68 mL, 4.03 mmol) at 0° C. The reaction mixture was then heated to reflux and stirred for 2 hours. The reaction was then cooled to 0° C., then quenched with water (1.0 mL), then a 10% w/v aqueous NaOH solution (1.0 mL) followed by water (2.0 mL). MgSO₄ (ca. 2 g) was added then the reaction mixture was through celite and the filter cake was washed with EtOAc (20 mL). The filtrate was concentrated to dryness under reduced pressure. The crude was purified by column chromatography eluting with a gradient of CH₂Cl₂ to 1:4 MeOH:CH—₂Cl₂ to afford the title compound as a solid (78 mg, 0.22 mmol, 54%.)

¹H NMR (400 MHz, DMSO-d₆) δ 11.94 (s, 1H), 9.44 (s, 1H), 7.88 (d, J=5.9 Hz, 1H), 6.24 (s, 1H), 6.20 (s, 1H), 4.53-4.45 (m, 1H), 4.39-4.31 (m, 1H), 3.94-3.86 (m, 1H), 3.55-3.42 (m, 2H), 3.06-2.84 (m, 2H), 2.71-2.60 (m, 2H), 2.63-2.54 (m, 1H), 2.32 (s, 3H), 2.05-1.93 (m, 2H), 1.76-1.50 (m, 6H).

UPLC-MS (basic method, 4 min): Rt: 1.30 min, m/z=358.4 [M+H]⁺.

### Example 93. Synthesis of Compound 127

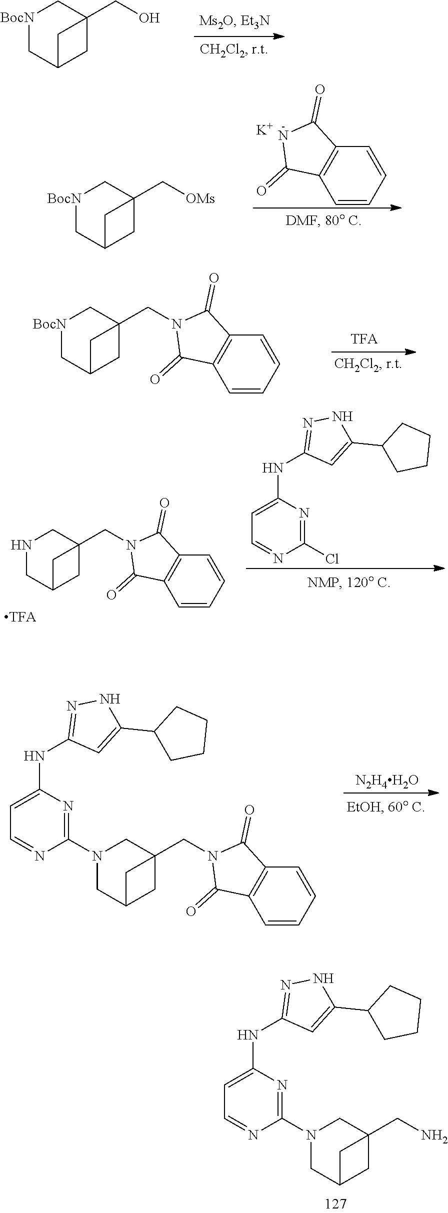

### tert-Butyl 1-(Methylsulfonyloxymethyl)-3-azabicyclo[3.1.1]heptane-3-carboxylate

To a solution of tert-butyl 1-(hydroxymethyl)-3-azabicyclo[3.1.1]heptane-3-carboxylate (500 mg, 2.20 mmol) in CH₂Cl₂ (20 mL) cooled to 0° C. was added Triethylamine (0.92 mL, 6.60 mmol), followed by methylsulfonic anhydride (766 mg, 4.40 mmol). The reaction was allowed to warm to room temperature and stirred for 16 h. Water (25 mL) was added to the reaction, the layers partitioned, and the organics were extracted with DCM (2×25 mL). The combined organics were washed with saturated brine solution (25 mL), dried with Na₂SO₄, filtered and concentrated to dryness to afford the title compound as a solid (646 mg, 2.09 mmol, 95%).

¹H NMR (400 MHz, Chloroform-d) δ 4.07 (t, J=2.2 Hz, 2H), 3.53 (dt, J=8.0, 2.2 Hz, 2H), 3.47 (dd, J=13.3, 1.7 Hz, 2H), 3.02 (dd, J=8.2, 1.8 Hz, 3H), 2.56-2.36 (m, 1H), 2.03-1.96 (m, 2H), 1.61 (s, 2H), 1.49 (s, 9H).

UPLC-MS (basic method, 4 min): rt=1.67 min, m/z: 306.1 [M+H]⁺.

### tert-Butyl 1-[(1,3-dioxoisoindolin-2-yl)methyl]-3-azabicyclo[3.1.1]heptane-3-carboxylate

To a solution of tert-butyl 1-(methylsulfonyloxymethyl)-3-azabicyclo[3.1.1]heptane-3-carboxylate (562 mg, 1.84 mmol) in DMF (20 mL) was added potassium phthalimide (375 mg, 2.02 mmol). The reaction was heated to 80° C. and stirred for 16 h. The reaction mixture was poured into ice water (200 mL), and the white precipitate allowed to settle, and the liquid was decanted. EtOAc (50 mL) was then added, and the resulting solution was washed sequentially with water (2×50 mL) and saturated brine solution (50 mL). The organic fraction was dried over Na₂SO₄ and concentrated to dryness to afford the title compound as a solid (563 mg, 1.44 mmol, 78%).

¹H NMR (400 MHz, Chloroform-d) δ 7.91-7.79 (m, 2H), 7.79-7.68 (m, 2H), 3.65 (d, J=12.1 Hz, 2H), 3.47 (dd, J=8.5, 2.6 Hz, 2H), 3.43 (s, 2H), 2.36 (dtt, J=21.5, 6.2, 2.5 Hz, 1H), 1.96 (dtd, J=9.7, 6.5, 2.4 Hz, 2H), 1.46 (dd, J=7.2, 0.8 Hz, 9H), 1.41 (td, J=7.7, 2.9 Hz, 2H).

UPLC-MS (basic method, 4 min): rt=1.23 min, m/z: 257.1 [M−Boc+H]⁺.

### 2-(3-Azoniabicyclo[3.1.1]heptan-1-ylmethyl)isoindoline-1,3-dione;2,2,2-trifluoroacetate

To a solution of tert-butyl 1-[(1,3-dioxoisoindolin-2-yl)methyl]-3-azabicyclo[3.1.1]heptane-3-carboxylate (554 mg, 1.55 mmol) in CH₂Cl₂ (20 mL) at 0° C. was added trifluoroacetic acid (4.0 mL, 52 mmol) and the resulting solution stirred at room temperature for 30 minutes. The reaction mixture was then concentrated to dryness under reduced pressure to afford the title compound as a solid (565 mg, 1.50 mmol, 96%).

¹H NMR (400 MHz, Chloroform-d) δ 9.46 (s, 2H), 7.87-7.80 (m, 2H), 7.74 (ddd, J=5.7, 3.0, 0.7 Hz, 2H), 3.68 (s, 2H), 3.43 (s, 4H), 2.52 (t, J=6.7 Hz, 1H), 2.24 (t, J=8.4 Hz, 2H), 1.80-1.71 (m, 2H).

UPLC-MS (basic method, 4 min): rt=1.09 min, m z: 370.3 [M+H]⁺.

### 2-[[3-[4-[(5-Cyclopentyl-1H-pyrazol-3-yl)amino]pyrimidin-2-yl]-3-azabicyclo[3.1.1]heptan-1-yl]methyl]isoindoline-1,3-dione

A solution of 2-chloro-N-(5-cyclopentyl-1H-pyrazol-3-yl)pyrimidin-4-amine (152 mg, 0.576 mmol) and 2-(3-azoniabicyclo[3.1.1]heptan-1-ylmethyl)isoindoline-1,3-dione;2,2,2-trifluoroacetate (256 mg, 0.692 mmol) and N,N-diisopropylethylamine (0.50 mL, 2.9 mmol) in NMP (5 mL) was heated to 120° C. and stirred for 72 hours. The reaction mixture was allowed to cool to room temperature and poured into ice water (400 mL), forming a precipitate which was collected by filtration. The filter cake was washed with water (2×10 mL), then dried under reduced pressure. The crude material was purified by normal-phase column chromatography, over silica gel, eluting with a gradient of CH₂Cl₂ to 1:9 0.1% Et₃N in MeOH:CH₂Cl₂. The residue was then purified by reversed-phase basic prepHPLC, to afford the title compound as a solid (77 mg, 0.14 mmol, 23%).

¹H NMR (400 MHz, DMSO-d₆) δ 12.08 (s, 1H), 7.95-7.80 (m, 5H), 6.70 (s, 1H), 6.42 (d, J=7.2 Hz, 1H), 3.79 (s, 2H), 3.62 (t, J=8.1 Hz, 4H), 3.35-3.28 (m, 1H), 3.08-3.00 (m, 1H), 2.03 (s, 4H), 1.75-1.46 (m, 8H).

UPLC-MS (basic method, 4 min): rt=1.18 min, m z: 484.2 [M+H]⁺.

### 2-[[3-[4-[(5-Cyclopentyl-1H-pyrazol-3-yl)amino]pyrimidin-2-yl]-3-azabicyclo[3.1.1]heptan-1-yl]methyl]isoindoline-1,3-dione

To a solution of 2-[[3-[4-[(5-cyclopentyl-1H-pyrazol-3-yl)amino]pyrimidin-2-yl]-3-azabicyclo[3.1.1]heptan-1-yl]methyl]isoindoline-1,3-dione (77 mg, 0.14 mmol) in ethanol (10 mL) was added hydrazine hydrate (68 μL, 1.4 mmol), then the reaction was heated to 60° C. and stirred for 5 hours. The reaction mixture was filtered, and the solid was washed with ethanol (10 mL). The combined filtrates were concentrated to dryness under reduced pressure. The residue was loaded onto an SCX-2 cartridge, the cartridge was washed with MeOH, then the compound was eluted with a 2 M solution of NH₃ in MeOH. The eluent was concentrated to dryness under reduced pressure. The residue was further purified by reversed-phase column chromatography, over C18 modified silica gel, eluting with a gradient of 1:19 MeCN (0.1% NH₃):water (0.1% NH₃) to 19:1 MeCN (0.1% NH₃):water (0.1% NH₃) to afford the title compound as a solid (7.2 mg, 19 μmol, 14%).

¹H NMR (400 MHz, DMSO-d₆) δ 11.82 (s, 1H), 9.36 (s, 1H), 7.83 (d, J=5.7 Hz, 1H), 6.39 (s, 1H), 6.18 (s, 1H), 3.70-3.54 (m, 2H), 3.55-3.41 (m, 2H), 3.02-2.90 (m, 1H), 2.50 (s, 2H), 2.44-2.39 (m, 1H), 2.01-1.81 (m, 4H), 1.72-1.47 (m, 8H), 1.25-1.17 (m, 2H).

UPLC-MS (basic method, 4 min): rt=1.44 min, m z: 354.4 [M+H]⁺.

### Example 94. Synthesis of Compound 126

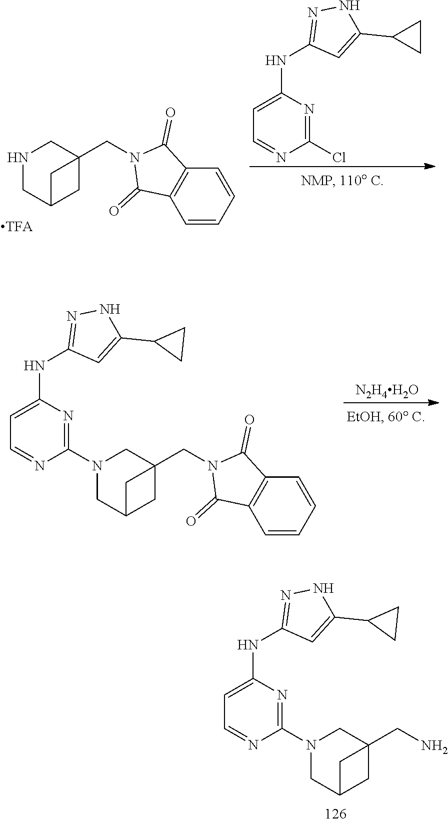

### 2-[[3-[4-[(5-Cycloprop-1H-pyrazol-3-yl)amino]pyrimidin-2-yl]-3-azabicyclo[3.1.1]heptan-1-yl]methyl]isoindoline-1,3-dione

To a solution of 2-chloro-N-(5-cyclopropyl-1H-pyrazol-3-yl)pyrimidin-4-amine (322 mg, 1.37 mmol) and 2-(3-azoniabicyclo[3.1.1]heptan-1-ylmethyl)isoindoline-1,3-dione;2,2,2-trifluoroacetate (607 mg, 1.64 mmol) in NMP (12 mL) was added N,N-diisopropylethylamine (1.2 mL, 6.8 mmol). The resulting solution was then heated to 110° C. and stirred for 16 h. The reaction mixture was poured into ice water (250 mL), and the solid that precipitated was filtered and washed with water (100 mL) and dried under reduced pressure to afford the title compound as powder (497 mg, 0.927 mmol, 68%).

¹H NMR (400 MHz, DMSO-d₆) δ 11.94-11.74 (m, 1H), 9.37 (s, 1H), 7.93-7.82 (m, 5H), 6.61-6.04 (m, 2H), 3.30 (t, J=7.0 Hz, 2H), 2.69 (d, J=1.1 Hz, 2H), 2.18 (t, J=8.1 Hz, 2H), 1.90 (dq, J=8.5, 7.4 Hz, 2H), 1.38 (d, J=7.8 Hz, 2H), 0.95 (d, J=6.9 Hz, 2H).

UPLC-MS (basic method, 4 min): rt=1.76 min, m z: 456.3 [M+H]⁺.

### 2-[[3-[4-[(5-Cyclopropyl-1H-pyrazol-3-yl)amino]pyrimidin-2-yl]-3-azabicyclo[3.1.1]heptan-1-yl]methyl]isoindoline-1,3-dione

To a solution of 2-[[3-[4-[(5-cyclopropyl-1H-pyrazol-3-yl)amino]pyrimidin-2-yl]-3-azabicyclo[3.1.1]heptan-1-yl]methyl]isoindoline-1,3-dione (364 mg, 0.615 mmol) in ethanol (15 mL) was added hydrazine hydrate (0.60 mL, 6.2 mmol), then the reaction was heated to 60° C. and stirred for 16 hours. The reaction mixture was allowed to cool to room temperature, then filtered, and the filter cake was washed with ethanol (10 mL). The combined filtrates were concentrated to dryness under reduced pressure. The residue was purified by reversed-phase basic prepHPLC. The residue was then dissolved in methanol (2 mL) then loaded on to an SCX-2 cartridge. The cartridge was washed with MeOH (20 mL) then the compound was eluted with 2 M NH₃ in MeOH (20 mL). The eluent was concentrated under reduced pressure to afford the title compound as a solid (44 mg, 0.14 mmol, 22%).

¹H NMR (400 MHz, DMSO-d₆) δ 11.90 (s, 1H), 9.37 (s, 1H), 7.87 (dd, J=5.6, 1.8 Hz, 1H), 6.24 (s, 2H), 3.64 (d, J=18.3 Hz, 2H), 3.53 (s, 2H), 2.56 (s, 2H), 1.99-1.77 (m, 4H), 1.27 (s, 2H), 0.95-0.86 (m, 2H), 0.72-0.60 (m, 2H).

UPLC-MS (basic method, 4 min): rt=1.22 min, m z: 326.3 [M+H]⁺.

### Example 95. Synthesis of Compound 123-1

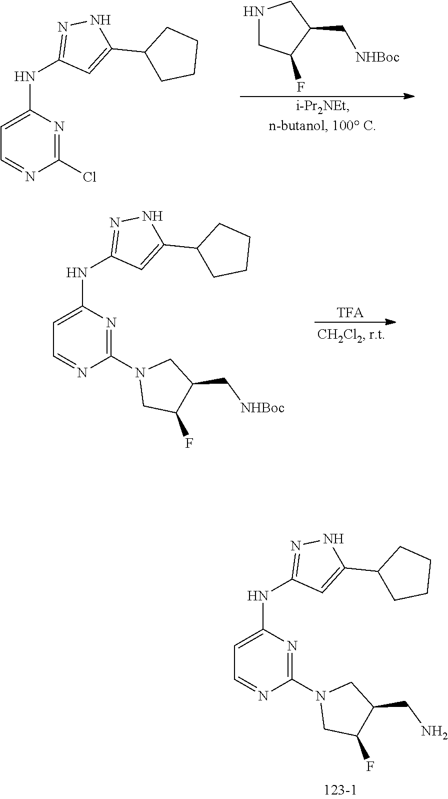

### tert-Butyl N-[[(3R,4R)-1-[4-[(5-Cyclopentyl-1H-pyrazol-3-yl)amino]pyrimidin-2-yl]-4-fluoro-pyrrolidin-3-yl]methyl]carbamate

A solution of 2-chloro-N-(5-cyclopentyl-1H-pyrazol-3-yl)pyrimidin-4-amine (200 mg, 0.758 mmol), tert-butyl N-[[(3S,4R)-4-fluoropyrrolidin-3-yl]methyl]carbamate (215 mg, 0.986 mmol), and N,N-diisopropylethylamine (260 μL, 1.52 mmol) in n-butanol (5 mL) was heated to 100° C. and stirred for 20 hours. The reaction mixture was concentrated to dryness under reduced pressure then the residue was suspended with water (40 mL), then extracted with CH₂Cl₂ (3×20 mL). The combined organic extracts were dried over MgSO₄, then concentrated to dryness under pressure to afford the title compound as a solid (330 mg, 0.718 mmol, 95%).

UPLC-MS (basic method, 4 min): Rt: 1.80 min, m/z=446.3 [M+H]⁺.

¹H NMR (400 MHz, DMSO-d₆) δ 11.90 (s, 1H), 9.42 (s, 1H), 7.85 (d, J=5.7 Hz, 1H), 7.02 (s, 1H), 6.29 (s, 2H), 5.34-5.15 (m, 1H), 3.88-3.73 (m, 2H), 3.26-3.10 (m, 3H), 3.01 (p, J=7.8 Hz, 1H), 2.05-1.93 (m, 2H), 1.78-1.49 (m, 7H), 1.40 (d, J=1.1 Hz, 10H).

¹⁹F NMR (376 MHz, DMSO-d₆) δ −191.1-−195.2 (m).

### 2-[(3R,4R)-3-(Aminomethyl)-4-fluoro-pyrrolidin-1-yl]-N-(5-cyclopentyl-1H-pyrazol-3-yl)pyrimidin-4-amine

To a solution of tert-butyl N-[[(3R,4R)-1-[4-[(5-cyclopentyl-1H-pyrazol-3-yl)amino]pyrimidin-2-yl]-4-fluoro-pyrrolidin-3-yl]methyl]carbamate (127 mg, 0.285 mmol) in CH₂Cl₂ (3 mL) was added trifluoroacetic acid (3.0 mL, 39 mmol) at 0° C. The reaction mixture was warmed to room temperature and stirred for 30 minutes. The reaction mixture was concentrated to dryness under reduced pressure. The residue was dissolved in MeOH (2 mL), then loaded onto an SCX-2 cartridge. The cartridge was washed with MeOH (40 mL), then the compound was eluted with 2 M NH₃ in MeOH (40 mL). The eluent was concentrated to dryness under reduced pressure to afford the title compound as a solid (90 mg, 0.26 mmol, 90%).

UPLC-MS (basic method, 4 min): Rt: 1.33 min, m/z=346.2 [M+H]⁺.

¹H NMR (400 MHz, DMSO-d₆) δ 11.90 (s, 1H), 9.41 (s, 1H), 7.85 (d, J=5.7 Hz, 1H), 6.27 (s, 2H), 5.62-4.97 (m, 1H), 4.55 (s, 1H), 4.02-3.70 (m, 1H), 3.15 (t, J=10.8 Hz, 1H), 3.07-2.86 (m, 2H), 2.81 (dd, J=12.6, 6.7 Hz, 1H), 2.57-2.51 (m, 5H), 1.97 (t, J=9.9 Hz, 2H), 1.78-1.47 (m, 5H).

¹⁹F NMR (376 MHz, DMSO-d₆) δ −191.9-−194.2 (m).

### Example 96. Synthesis of Compound 123-2

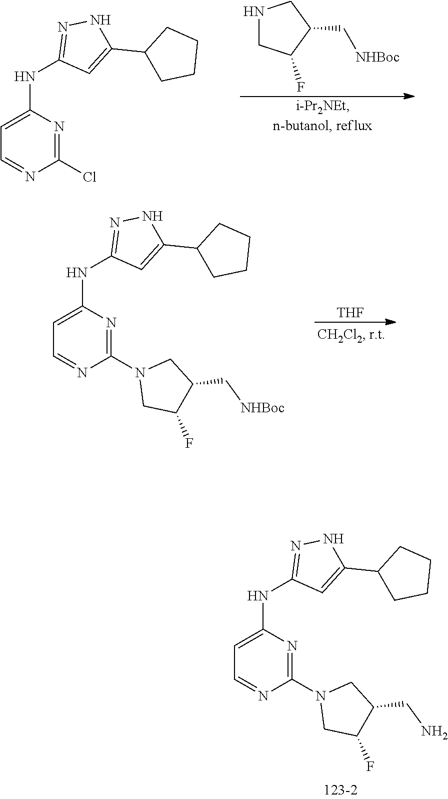

### tert-Butyl N-[[(3S,4S)-1-[4-[(5-Cyclopentyl-1H-pyrazol-3-yl)amino]pyrimidin-2-yl]-4-fluoro-pyrrolidin-3-yl]methyl]carbamate

A solution of 2-chloro-N-(5-cyclopentyl-1H-pyrazol-3-yl)pyrimidin-4-amine (280 mg, 1.06 mmol), tert-butyl N-[[(3R,4S)-4-fluoropyrrolidin-3-yl]methyl]carbamate (250 mg, 1.15 mmol), and N,N-diisopropylethylamine (0.46 mL, 2.7 mmol) in n-butanol (5 mL) was heated to reflux and stirred for 2 h. Reaction mixture was cooled to room temperature then concentrated to dryness under reduced pressure. The residue was suspended in water (50 mL) then extracted with 1:4 MeOH in DCM (2×50 mL). The combined organic extracts were dried over Na₂SO₄ then concentrated to dryness under reduced pressure to afford the title compound as an oil (462 mg, 1.04 mmol, 98%).

UPLC-MS (basic method, 2 min): Rt: 1.08 min, m/z=446.3 [M+H]⁺.

¹H NMR (400 MHz, DMSO-d₆) δ 11.86 (s, 1H), 9.37 (s, 1H), 7.81 (d, J=5.8 Hz, 1H), 6.99 (s, 1H), 6.27 (s, 2H), 5.23 (d, J=53.9 Hz, 1H), 3.87-3.69 (m, 2H), 3.24-3.04 (m, 4H), 2.98 (q, J=7.9 Hz, 1H), 1.95 (s, 2H), 1.62 (d, J=46.1 Hz, 6H), 1.36 (s, 9H), 1.25-1.16 (m, 1H).

### 2-[(3S,4S)-3-(Aminomethyl)-4-fluoro-pyrrolidin-1-yl]-N-(5-cyclopentyl-1H-pyrazol-3-yl)pyrimidin-4-amine

A solution of tert-butyl N-[[(3S,4S)-1-[4-[(5-cyclopentyl-1H-pyrazol-3-yl)amino]pyrimidin-2-yl]-4-fluoro-pyrrolidin-3-yl]methyl]carbamate (300 mg, 0.673 mmol) was dissolved in CH₂Cl₂ (6 mL) and trifluoroacetic acid (0.52 mL, 6.7 mmol) was added to it at 0° C. The reaction mixture was kept at 0° C. and stirred for 2 hours. The reaction mixture was then concentrated to dryness under reduced pressure. The residue was dissolved in MeOH, then loaded on to an SCX-2 cartridge. The cartridge was washed with MeOH (10 mL), then the compound was eluted with 2 M NH₃ in MeOH (20 mL). The eluent was concentrated to dryness under reduced pressure to afford the title compound as a solid (145 mg, 0.403 mmol, 60%).

UPLC-MS (basic method, 4 min): Rt: 1.34 min, m/z=346.2 [M+H]+.

¹H NMR (400 MHz, DMSO-d₆) δ 11.85 (s, 1H), 9.38 (s, 1H), 7.81 (d, J=5.7 Hz, 1H), 6.31 (s, 1H), 6.21 (s, 1H), 5.41-5.18 (m, 1H), 3.90-3.72 (m, 2H), 3.01 (s, 1H), 2.96 (q, J=7.6 Hz, 1H), 2.81 (dd, J=12.4, 7.9 Hz, 1H), 2.68 (dd, J=12.4, 6.6 Hz, 1H), 2.45-2.26 (m, 1H), 2.03-1.85 (m, 2H), 1.73-1.47 (m, 7H).

¹⁹F NMR (400 MHz, DMSO-d₆) δ −192.7-193.8 (m).

### Example 97. Synthesis of Compound 122-1

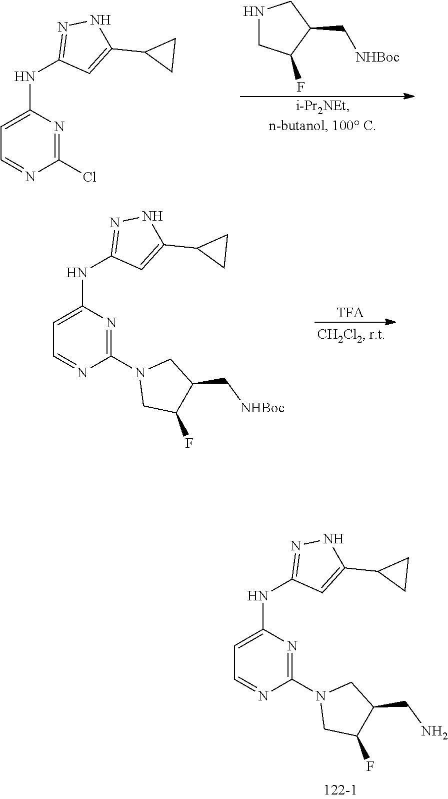

### tert-Butyl N-[[(3R,4R)-1-[4-[(5-Cyclopropyl-1H-pyrazol-3-yl)amino]pyrimidin-2-yl]-4-fluoro-pyrrolidin-3-yl]methyl]carbamate

A solution of 2-chloro-N-(5-cyclopropyl-1H-pyrazol-3-yl)pyrimidin-4-amine (200 mg, 0.849 mmol), tert-butyl N-[[(3S,4R)-4-fluoropyrrolidin-3-yl]methyl]carbamate (278 mg, 1.27 mmol), and N,N-diisopropylethylamine (370 μL, 2.12 mmol) in n-butanol (5 mL) was heated to 100° C. and stirred for 72 hours. The reaction mixture was concentrated to dryness under reduced pressure then the residue was suspended with water (40 mL), then extracted with CH₂Cl₂ (3×20 mL). The combined organic extracts were dried over MgSO₄, then concentrated to dryness under pressure to afford the title compound as a solid (348 mg, 0.817 mmol, 96%).

UPLC-MS (basic method, 4 min): Rt: 1.57 min, m/z=418.3 [M+H]⁺.

¹H NMR (400 MHz, DMSO-d₆) δ 11.90 (s, 1H), 9.39 (s, 1H), 7.85 (d, J=5.7 Hz, 1H), 7.04 (s, 1H), 6.26 (s, 1H), 6.14 (s, 1H) 5.58-4.92 (m, 1H), 3.86-3.71 (m, 2H), 3.27-3.07 (m, 4H), 1.87 (tq, J=8.7, 4.2 Hz, 1H), 1.40 (s, 10H), 0.95-0.88 (m, 2H), 0.74-0.61 (m, 2H).

¹⁹F NMR (376 MHz, DMSO-d₆) δ −192.5-193.1 (m).

### 2-[(3R,4R)-3-(Aminomethyl)-4-fluoro-pyrrolidin-1-yl]-N-(5-cyclopropyl-1H-pyrazol-3-yl)pyrimidin-4-amine

To a solution of tert-butyl N-[[(3R,4R)-1-[4-[(5-cyclopropyl-1H-pyrazol-3-yl)amino]pyrimidin-2-yl]-4-fluoro-pyrrolidin-3-yl]methyl]carbamate (112 mg, 0.263 mmol) in CH₂Cl₂ (3 mL) was added trifluoroacetic acid (3.0 mL, 39 mmol) at 0° C. The reaction mixture was warmed to room temperature and stirred for 30 minutes. The reaction mixture was concentrated to dryness under reduced pressure. The residue was dissolved in MeOH (2 mL), then loaded onto an SCX-2 cartridge. The cartridge was washed with MeOH (40 mL), then the compound was eluted with 2 M NH₃ in MeOH (40 mL). The eluent was concentrated to dryness under reduced pressure to afford the title compound as a solid (62 mg, 0.19 mmol, 74%).

UPLC-MS (basic method, 4 min): Rt: 1.06 min, m/z=318.3 [M+H]⁺.

¹H NMR (400 MHz, DMSO-d₆) δ 11.91 (s, 1H), 9.39 (s, 1H), 7.85 (d, J=5.7 Hz, 1H), 6.23 (s, 2H), 5.52-5.07 (m, 1H), 3.89-3.77 (m, 1H), 3.12 (t, J=11.1 Hz, 1H), 2.88 (dd, J=12.4, 7.8 Hz, 1H), 2.75 (dd, J=12.3, 6.8 Hz, 1H), 2.54 (d, J=0.6 Hz, 5H), 1.87 (ddd, J=13.4, 8.5, 4.9 Hz, 1H), 1.20-0.80 (m, 2H), 0.75-0.53 (m, 2H).

¹⁹F NMR (376 MHz, DMSO-d₆) δ −192.7-−193.7 (m).

### Example 98. Synthesis of Compound 122-2

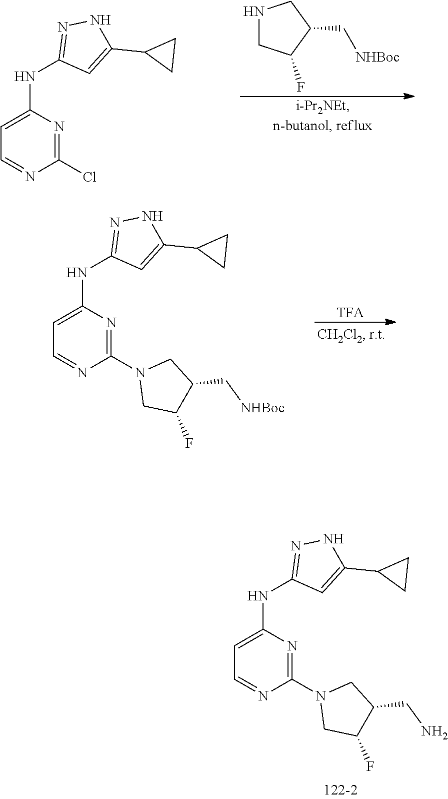

### tert-Butyl N-[[(3S,4S)-1-[4-[(5-cyclopropyl-1H-pyrazol-3-yl)amino]pyrimidin-2-yl]-4-fluoro-pyrrolidin-3-yl]methyl]carbamate

A solution of 2-chloro-N-(5-cyclopropyl-1H-pyrazol-3-yl)pyrimidin-4-amine (250 mg, 1.06 mmol), tert-butyl N-[[(3R,4S)-4-fluoropyrrolidin-3-yl]methyl]carbamate (250 mg, 1.15 mmol), and N,N-diisopropylethylamine (0.46 mL, 2.7 mmol) in n-butanol (5 mL) was heated at reflux for 2 h. The reaction mixture was cooled to room temperature, then concentrated to dryness under reduced pressure. The residue was suspended in water (10 mL) then extracted with 20% MeOH in DCM (2×50 mL). The combined organic extracts were dried over Na₂SO₄ then concentrated under reduced pressure to afford the title compound as a solid (437 mg, 1.05 mmol, 99%).

UPLC-MS (basic method, 2 min): Rt: 1.00 min, m/z=418.3 [M+H]⁺.

¹H NMR (400 MHz, DMSO-d₆) δ 11.87 (s, 1H), 9.37 (s, 1H), 7.81 (d, J=5.8 Hz, 1H), 7.00 (s, 1H), 6.20 (s, 1H), 5.23 (d, J=53.7 Hz, 1H), 3.86-3.65 (m, 2H), 3.24-3.09 (m, 2H), 1.82 (td, J=8.6, 4.4 Hz, 1H), 1.36 (s, 9H), 1.31-1.13 (m, 3H), 0.84 (s, 2H), 0.69-0.56 (m, 2H).

### 2-[(3S,4S)-3-(Aminomethyl)-4-fluoro-pyrrolidin-1-yl]-N-(5-cyclopropyl-1H-pyrazol-3-yl)pyrimidin-4-amine

To a solution of tert-butyl N-[[(3S,4S)-1-[4-[(5-cyclopropyl-1H-pyrazol-3-yl)amino]pyrimidin-2-yl]-4-fluoro-pyrrolidin-3-yl]methyl]carbamate (300 mg, 0.719 mmol) in CH₂Cl₂ (6 mL) was added trifluoroacetic acid (0.55 mL, 7.2 mmol) at 0° C. The reaction mixture was warmed to room temperature and stirred for 2 h. The reaction mixture was then concentrated to dryness under reduced pressure. The residue was dissolved in MeOH, then loaded on to an SCX-2 cartridge. The cartridge was washed with MeOH (10 mL), then the compound was eluted with 2 M NH₃ in MeOH (20 mL). The eluent was concentrated to dryness under reduced pressure to afford the title compound as a solid (150 mg, 0.444 mmol, 62%).

UPLC-MS (basic method, 4 min): Rt: 1.12 min, m/z=318.2 [M+H]⁺.

¹H NMR (400 MHz, DMSO-d₆) δ 11.87 (s, 1H), 9.38 (s, 1H), 7.81 (d, J=5.7 Hz, 1H), 6.18 (s, 2H), 5.30 (d, J=53.7 Hz, 1H), 3.91-3.71 (m, 4H), 3.10 (t, J=10.8 Hz, 1H), 2.90 (dd, J=12.5, 7.6 Hz, 1H), 2.77 (dd, J=12.6, 6.8 Hz, 1H), 1.83 (tt, J=8.8, 5.0 Hz, 1H), 0.87 (qt, J=7.3, 3.6 Hz, 2H), 0.67-0.51 (m, 2H).

¹⁹F NMR (376 MHz, DMSO-d₆) δ −196.6-−197.3 (m).

### Example 99. Synthesis of Compound 125

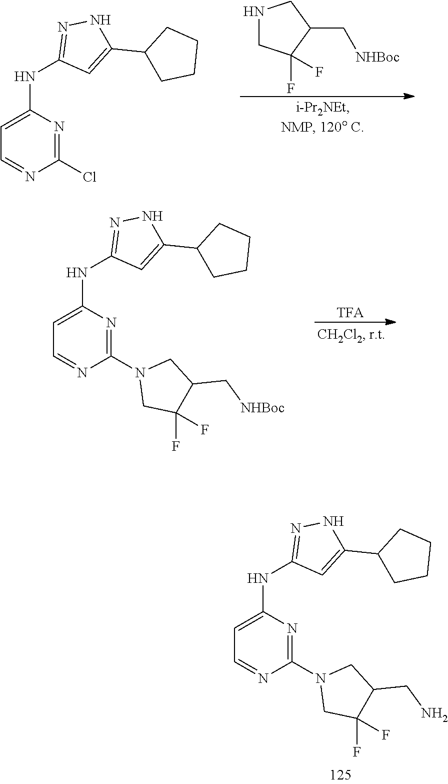

### tert-Butyl N-[[1-[4-[(5-Cyclopentyl-1H-pyrazol-3-yl)amino]pyrimidin-2-yl]-4,4-difluoro-pyrrolidin-3-yl]methyl]carbamate

A solution of tert-butyl ((4,4-difluoropyrrolidin-3-yl)methyl)carbamate (205 mg, 0.866 mmol), 2-chloro-N-(5-cyclopentyl-1H-pyrazol-3-yl)pyrimidin-4-amine (190 mg, 0.721 mmol) and N,N-diisopropylethylamine (314 μL, 1.80 mmol) in NMP (2 mL) was heated to 120° C. and stirred for 48 hours. The reaction mixture was cooled to room temperature, then added dropwise to ice water (ca. 50 mL) resulting in the formation of a precipitate. The precipitate was collected by filtration, and the filter cake was washed with water (2×10 mL), then dried under reduced pressure to afford the title compound as a solid (297 mg, 0.641 mmol, 89%).

UPLC-MS (basic method, 4 min): Rt: 1.87 min, m/z=464.4 [M+H]⁺.

¹H NMR (400 MHz, DMSO-d₆) δ 11.92 (s, 1H), 9.50 (s, 1H), 7.88 (d, J=5.8 Hz, 1H), 7.05 (t, J=5.3 Hz, 1H), 6.45-6.18 (m, 2H), 3.96-3.80 (m, 3H), 3.43-3.34 (m, 1H), 3.30-3.25 (m, 1H), 3.14-2.95 (m, 2H), 2.95-2.77 (m, 1H), 2.04-1.92 (m, 2H), 1.78-1.50 (m, 6H), 1.38 (s, 9H)

¹⁹F NMR (376 MHz, DMSO-d₆) δ −102.8-−105.2 (m), −111.0-−112.7 (m).

### 2-[4-(Aminomethyl)-3,3-difluoro-pyrrolidin-1-yl]-N-(5-cyclopentyl-1H-pyrazol-3-yl)pyrimidin-4-amine

To a solution of tert-butyl N-[[1-[4-[(5-cyclopentyl-1H-pyrazol-3-yl)amino]pyrimidin-2-yl]-4,4-difluoro-pyrrolidin-3-yl]methyl]carbamate (100 mg, 0.216 mmol) in CH₂Cl₂ (2.5 mL) was added trifluoroacetic acid (2.5 mL, 33 mmol) and the reaction mixture was stirred for 2 hours. The reaction mixture was then concentrated to dryness under reduced pressure. The residue was dissolved in MeOH, then loaded onto an SCX-2 cartridge. The cartridge was washed with MeOH (25 mL), then the compound was eluted with 2 M NH₃ in MeOH (25 mL). The eluent was concentrated to dryness under reduced pressure to afford the title compound as a solid (72 mg, 0.20 mmol, 92%).

UPLC-MS (basic method, 4 min): Rt: 1.40 min, m/z=364.4 [M+H]⁺.

¹H NMR (400 MHz, Chloroform-d) δ 8.03 (d, J=5.6 Hz, 1H), 6.86 (s, 1H), 6.26 (d, J=5.4 Hz, 1H), 6.11 (s, 1H), 4.08-3.86 (m, 3H), 3.54-3.42 (m, 1H), 3.15 (dd, J=13.1, 7.1 Hz, 1H), 3.06 (p, J=8.0 Hz, 1H), 2.89 (dd, J=13.2, 6.7 Hz, 1H), 2.76-2.60 (m, 1H), 2.15-2.04 (m, 2H), 1.84-1.49 (m, 6H).

¹⁹F NMR (376 MHz, Chloroform-d) δ −102.0-−102.8 (m), −112.0-−112.8 (m).

### Example 100. Synthesis of Compound 124

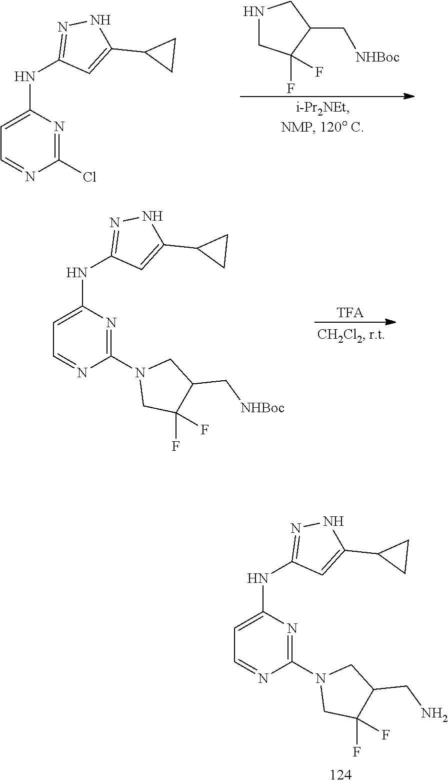

### tert-Butyl N-[[1-[4-[(5-Cyclopropyl-1H-pyrazol-3-yl)amino]pyrimidin-2-yl]-4,4-difluoro-pyrrolidin-3-yl]methyl]carbamate

A solution of 2-chloro-N-(5-cyclopropyl-1H-pyrazol-3-yl)pyrimidin-4-amine (170 mg, 0.721 mmol), tert-butyl ((4,4-difluoropyrrolidin-3-yl)methyl)carbamate (205 mg, 0.866 mmol), and N,N-diisopropylethylamine (314 μL, 1.80 mmol) was heated to 120° C. and stirred for 24 hours. The reaction mixture was cooled to room temperature, then added dropwise to stirring ice water (ca. 50 mL) resulting in the formation of a precipitate. The precipitate was collected by filtration, then the filter cake was washed with water (2×10 mL), then dried under reduced pressure to afford the title compound as a solid (254 mg, 0.583 mmol, 81%).

UPLC-MS (basic method, 2 min): Rt: 1.04 min, m/z=436.4 [M+H]⁺.

¹H NMR (400 MHz, DMSO-d₆) δ 11.93 (s, 1H), 9.49 (s, 1H), 7.88 (d, J=5.7 Hz, 1H), 7.07 (t, J=5.7 Hz, 1H), 6.46-5.97 (m, 2H), 4.00-3.78 (m, 3H), 3.43-3.35 (m, 1H), 3.32-3.23 (m, 1H), 3.15-3.01 (m, 1H), 2.96-2.76 (m, 1H), 1.87 (tt, J=8.8, 5.1 Hz, 1H), 1.38 (s, 9H), 0.99-0.85 (m, 2H), 0.72-0.61 (m, 2H).

¹⁹F NMR (376 MHz, DMSO-d₆) δ-−102.5-−104.9 (m), −111.0-−112.7 (m).

### 2-[4-(Aminomethyl)-3,3-difluoro-pyrrolidin-1-yl]-N-(5-cyclopropyl-1H-pyrazol-3-yl)pyrimidin-4-amine

To a solution of tert-butyl N-[[1-[4-[(5-cyclopropyl-1H-pyrazol-3-yl)amino]pyrimidin-2-yl]-4,4-difluoro-pyrrolidin-3-yl]methyl]carbamate (100 mg, 0.230 mmol) in CH₂Cl₂ (2 mL) was added trifluoroacetic acid (2.0 mL, 26 mmol) and the reaction mixture was stirred at room temperature for 2 hours. The reaction mixture was concentrated to dryness under reduced pressure. The residue was dissolved in MeOH (2 mL), then loaded onto an SCX-2 cartridge. The cartridge was washed with MeOH (25 mL), then the compound was eluted with 2 M NH₃ in MeOH (25 mL). The eluent was concentrated to dryness under reduced pressure to afford the title compound as a solid (75 mg, 0.22 mmol, 97%).

UPLC-MS (basic method, 4 min): Rt: 1.19 min, m/z=336.3 [M+H]⁺.

¹H NMR (400 MHz, DMSO-d₆) δ 11.94 (s, 1H), 9.49 (s, 1H), 7.88 (d, J=5.7 Hz, 1H), 6.30 (s, 1H), 6.20 (s, 1H), 3.97-3.81 (m, 3H), 3.43 (dd, J=11.2, 7.0 Hz, 1H), 2.99-2.89 (m, 1H), 2.77-2.58 (m, 2H), 1.87 (tt, J=8.4, 5.0 Hz, 1H), 0.95-0.86 (m, 2H), 0.71-0.62 (m, 2H).

¹⁹F NMR (376 MHz, DMSO-d₆) δ−101.2-−102.8 (m), −111.1-−112.5 (m).

### Example 101. Synthesis of Compound 181

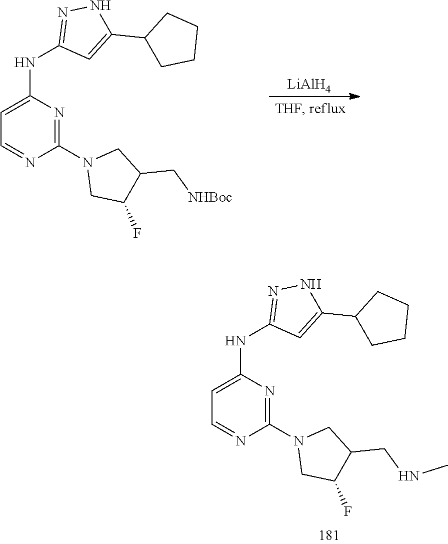 [SMILES: CC(C)(C)OC(=O)NCC1CN(c2nccc(Nc3cc(C4CCCC4)[nH]n3)n2)C[C@H]1F ; "LiAlH4" ; CNCC1CN(c2nccc(Nc3cc(C4CCCC4)[nH]n3)n2)C[C@H]1F]

To solution of tert-butyl N-[[(3S,4S)-1-[4-[(5-cyclopentyl-1H-pyrazol-3-yl)amino]pyrimidin-2-yl]-4-fluoro-pyrrolidin-3-yl]methyl]carbamate (130 mg, 0.292 mmol) in anhydrous THE (10 mL) under a nitrogen atmosphere was added a 2.4 M solution of lithium aluminum hydride in THE (1.2 mL, 2.92 mmol) dropwise at 0° C. The reaction mixture was then heated to reflux and stirred for 3 hours. The reaction mixture was cooled to 0° C. and then quenched by dropwise addition of saturated ammonium chloride solution (20 mL) in ice water bath. The aqueous layer was extracted with 20% MeOH in CH₂Cl₂ (2×25 mL). The combined organic extracts were washed with water (20 mL), a saturated aqueous brine solution (25 mL) dried over Na₂SO₄, then concentrated to dryness under reduced pressure. The residue was purified by reversed-phase basic prepHPLC to afford the title compound as a solid (30 mg, 78 μmol, 27%).

UPLC-MS (basic method, 4 min): Rt: 1.43 min, m/z=360.2 [M+H]⁺.

¹H NMR (400 MHz, DMSO-d₆) δ 11.85 (s, 1H), 9.37 (s, 1H), 7.81 (d, J=5.7 Hz, 1H), 6.21 (s, 2H), 5.24 (d, J=54.0 Hz, 1H), 3.77 (dd, J=25.5, 13.5 Hz, 2H), 3.63 (d, J=43.8 Hz, 1H), 3.10 (s, 1H), 2.97 (p, J=7.8 Hz, 1H), 2.74 (dd, J=11.2, 7.1 Hz, 1H), 2.56 (dd, J=11.8, 6.7 Hz, 1H), 2.29 (s, 3H), 2.01-1.88 (m, 2H), 1.76-1.42 (m, 7H).

¹⁹F NMR (376 MHz, DMSO-d₆) δ −192.3-−192.9 (m).

### Example 102: Synthesis of Compound 182

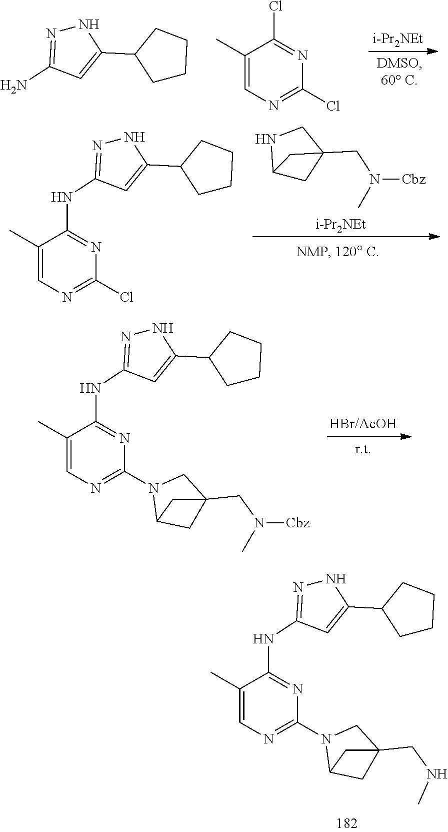

### 2-Chloro-N-(5-Cyclopentyl-1H-pyrazol-3-yl)-5-methyl-pyrimidin-4-amine

A solution of 5-cyclopentyl-1H-pyrazol-3-amine (510 mg, 3.37 mmol), 2,4-dichloro-5-methylpyrimidine (500 mg, 3.07 mmol) and N,N-diisopropylethylamine (0.80 mL, 4.60 mmol) in DMSO (5 mL) was heated to 60° C. and stirred for 18 hours. The reaction mixture was then allowed to cool to room temperature and then poured into ice water (ca. 200 mL) resulting in a precipitation. The precipitate was collected by filtration and the filter cake washed with water (20 mL) followed by CH₂Cl₂ (20 mL). The filter cake was dried under reduced pressure to afford the title compound as a solid (410 mg, 1.46 mmol, 48%).

H NMR (400 MHz, DMSO-d₆) δ 12.20 (s, 1H), 9.29 (s, 1H), 7.97 (s, 1H), 6.37 (s, 1H), 3.17-2.95 (m, 1H), 2.11 (d, J=0.9 Hz, 3H), 2.07-1.94 (m, 2H), 1.79-1.66 (m, 2H), 1.68-1.48 (m, 4H).

UPLC-MS (basic method, 2 min): Rt: 1.20 min, m/z: 278.1 [M+H]⁺.

### Benzyl N-[[2-[4-[(5-Cyclopentyl-1H-pyrazol-3-yl)amino]-5-methyl-pyrimidin-2-yl]-2-azabicyclo[2.1.1]hexan-4-yl]methyl]-N-methyl-carbamate

A solution of 2-chloro-N-(5-cyclopentyl-1H-pyrazol-3-yl)-5-methyl-pyrimidin-4-amine (150 mg, 0.540 mmol), benzyl N-(2-azabicyclo[2.1.1]hexan-4-ylmethyl)-N-methyl-carbamate (211 mg, 0.810 mmol) and N,N-diisopropylethylamine (0.28 mL, 1.6 mmol) in NMP (1.5 mL) was heated to 120° C. and stirred for 20 hours. The reaction mixture was then added dropwise to ice water (ca. 100 mL) resulting in the formation of a precipitate. The precipitate was collected by filtration, then washed with water (2×10 mL). The resultant solid was purified by normal-phase column chromatography, over silica gel, eluting with a gradient of CH₂Cl₂ to 95:5 CH₂Cl₂:0.1% NH₃ in MeOH to afford the title compound as a solid (154 mg, 0.307 mmol, 57%).

¹H NMR (400 MHz, chloroform-d) δ 7.80 (s, 1H), 7.37-7.23 (m, 5H), 6.87 (s, 1H), 5.11 (d, J=2.6 Hz, 2H), 4.75 (d, J=15.6 Hz, 1H), 3.59 (d, J=19.1 Hz, 2H), 3.47 (d, J=18.4 Hz, 2H), 3.12-3.01 (m, 1H), 2.98 (d, J=8.9 Hz, 3H), 2.07 (s, 2H), 2.02 (s, 3H), 1.92-1.76 (m, 4H), 1.76-1.61 (m, 4H), 1.61-1.44 (m, 2H)

UPLC-MS (basic method, 2 min): Rt: 1.18 min, m/z: 502.3 [M+H]⁺.

### N-(5-Cyclopentyl-1H-pyrazol-3-yl)-5-methyl-2-[4-(methylaminomethyl)-2-azabicyclo[2.1.1]hexan-2-yl]pyrimidin-4-amine

To benzyl N-[[2-[4-[(5-cyclopentyl-1H-pyrazol-3-yl)amino]-5-methyl-pyrimidin-2-yl]-2-azabicyclo[2.1.1]hexan-4-yl]methyl]-N-methyl-carbamate (140 mg, 0.279 mmol) was added a 30% solution of HBr in acetic acid (1.4 mL) and the reaction mixture stirred at room temperature for 30 minutes. The reaction mixture was concentrated to dryness under reduced pressure and loaded onto an SCX-2 cartridge. The cartridge was washed with MeOH (30 mL), then the compound was eluted with a 2 M solution of NH₃ in MeOH (30 mL). The eluent was concentrated to dryness under reduced pressure to afford the title compound as a solid (83 mg, 0.21 mmol, 77%).

¹H NMR (400 MHz, DMSO-d₆) δ 11.93 (s, 1H), 8.43 (s, 1H), 7.69 (s, 1H), 6.53 (s, 1H), 4.62 (d, J=1.8 Hz, 1H), 3.07-2.99 (m, 1H), 2.74 (s, 2H), 2.31 (s, 3H), 2.02-1.97 (m, 6H), 1.80-1.67 (m, 5H), 1.68-1.55 (m, 5H), 1.40-1.30 (m, 2H).

UPLC-MS (basic method, 4 min): Rt: 1.38 min, m/z: 368.4 [M+H]⁺.

### Example 103: Synthesis of Compound 183

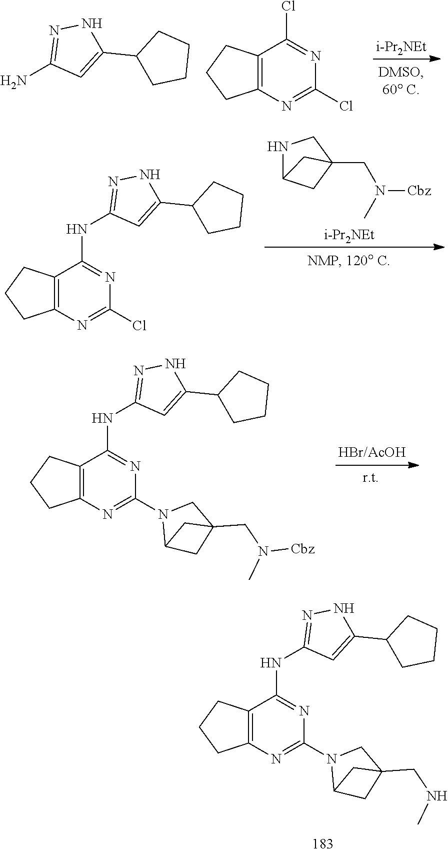

### 2-Chloro-N-(5-Cyclopentyl-1H-pyrazol-3-yl)-6,7-dihydro-5H-cyclopenta[d]pyrimidin-4-amine

A solution of 5-cyclopentyl-1H-pyrazol-3-amine (440 mg, 2.91 mmol), 2,4-dichloro-6,7-dihydro-5H-cyclopenta[d]pyrimidine (500 mg, 2.64 mmol) and N,N-diisopropylethylamine (0.69 mL, 4.0 mmol) in DMSO (5 mL) was heated to 60° C. and stirred for 18 hours. The reaction mixture was then allowed to cool to room temperature and then poured into ice water (ca. 200 mL) resulting in a precipitation. The precipitate was collected by filtration and the filter cake washed with water (2×20 mL). The filter cake was dried under reduced pressure to afford the title compound as a solid. (495 mg, 1.56 mmol, 59%).

¹H NMR (400 MHz, DMSO-d₆) δ 12.15 (s, 1H), 9.59 (s, 1H), 6.37 (s, 1H), 3.09-2.89 (m, 1H), 2.80-2.68 (m, 4H), 2.07-1.95 (m, 4H), 1.76-1.68 (m, 2H), 1.66-1.51 (m, 4H).

UPLC-MS (basic method, 2 min): Rt: 1.06 min, m/z: 304.1 [M+H]⁺.

### Benzyl N-[[2-[4-[(5-cyclopentyl-1H-pyrazol-3-yl)amino]-6,7-dihydro-5H-cyclopenta[d]pyrimidin-2-yl]-2-azabicyclo[2.1.1]hexan-4-yl]methyl]-N-methyl-carbamate

A solution of 2-chloro-N-(5-cyclopentyl-1H-pyrazol-3-yl)-6,7-dihydro-5H-cyclopenta[d]pyrimidin-4-amine (150 mg, 0.494 mmol), benzyl N-(2-azabicyclo[2.1.1]hexan-4-ylmethyl)-N-methyl-carbamate (154 mg, 0.593 mmol) and N,N-diisopropylethylamine (0.26 mL, 1.48 mmol) in NMP (1.5 mL) was heated to 120° C. and stirred for 20 hours. The reaction mixture was then added dropwise to ice water (ca. 100 mL) resulting in the formation of a precipitate. The precipitate was collected by filtration, then washed with water (2×10 mL). The resultant solid was purified by normal-phase column chromatography, over silica gel, eluting with a gradient of CH₂Cl₂ to 95:5 CH₂Cl₂:0.1% NH₃ in MeOH to afford the title compound as a solid (132 mg, 0.250 mmol, 51%).

¹H NMR (400 MHz, chloroform-d) δ 7.37-7.22 (m, 5H), 7.11 (s, 1H), 5.10 (d, J=5.2 Hz, 2H), 4.79 (d, J=16.1 Hz, 1H), 3.57 (d, J=19.4 Hz, 2H), 3.48 (d, J=17.6 Hz, 2H), 3.10-2.98 (m, 1H), 2.97 (d, J=9.5 Hz, 3H), 2.77 (t, J=7.7 Hz, 2H), 2.62 (t, J=7.3 Hz, 2H), 2.10-1.96 (m, 4H), 1.83-1.72 (m, 4H), 1.69-1.41 (m, 6H).

UPLC-MS (basic method, 2 min): Rt: 1.21 min, m/z: 528.3 [M+H]⁺.

### N-(5-Cyclopentyl-1H-pyrazol-3-yl)-2-[4-(methylaminomethyl)-2-azabicyclo[2.1.1]hexan-2-yl]-6,7-dihydro-5H-cyclopenta[d]pyrimidin-4-amine

To benzyl N-[[2-[4-[(5-cyclopentyl-1H-pyrazol-3-yl)amino]-6,7-dihydro-5H-cyclopenta[d]pyrimidin-2-yl]-2-azabicyclo[2.1.1]hexan-4-yl]methyl]-N-methyl-carbamate (122 mg, 0.231 mmol) was added a 30% solution of HBr in acetic acid (1.2 mL) and the reaction mixture stirred at room temperature for 45 minutes. The reaction mixture was concentrated to dryness under reduced pressure and loaded onto an SCX-2 cartridge. The cartridge was washed with MeOH (30 mL), then the compound was eluted with a 2 M solution of NH₃ in MeOH (30 mL). The eluent was concentrated to dryness under reduced pressure to afford the title compound as a solid (87 mg, 0.21 mmol, 93%).

¹H NMR (400 MHz, DMSO-d₆) δ 11.88 (s, 1H), 8.76 (s, 1H), 6.52 (s, 1H), 4.64 (t, J=1.8 Hz, 1H), 3.03 (q, J=7.7 Hz, 1H), 2.75 (s, 2H), 2.69-2.57 (m, 4H), 2.32 (s, 3H), 2.03-1.86 (m, 5H), 1.77 (d, J=4.3 Hz, 2H), 1.74-1.67 (m, 2H), 1.67-1.53 (m, 5H), 1.40-1.30 (m, 2H).

UPLC-MS (basic method, 4 min): Rt: 2.51 min, m/z: 394.4 [M+H]⁺.

### Example 104: Synthesis of Compound 121

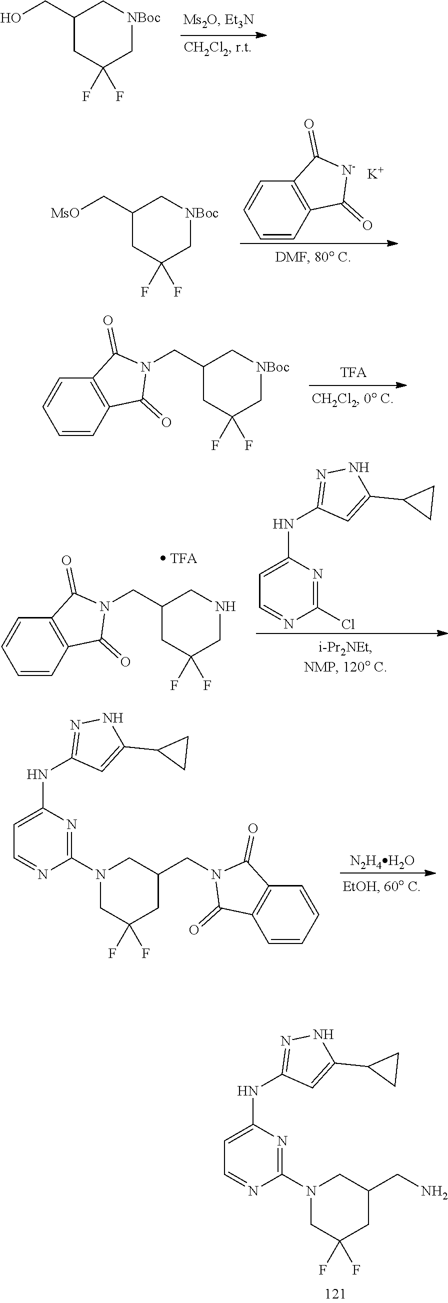

### tert-Butyl 3,3-Difluoro-5-(methylsulfonyloxymethyl)piperidine-1-carboxylate

To a solution of 3,3-difluoro-5-hydroxymethyl-piperidine-1-carboxylic acid tert-butyl ester (1.00 g, 3.98 mmol) and triethylamine (1.7 mL, 12 mmol) in CH₂Cl₂ (40 mL) was added methanesulfonic anhydride (1.39 g, 7.96 mmol) at 0° C. The reaction mixture was warmed to room temperature and stirred for 16 hours. The reaction mixture was then diluted with water (20 mL) and extracted with CH₂Cl₂ (3×20 mL). The combined organic extracts were washed with a saturated aqueous brine solution (30 mL), dried over Na₂SO₄, then concentrated under reduced pressure to afford the title compound as an oil (1.30 g, 3.95 mmol, 99%).

¹H NMR (400 MHz, Chloroform-d) δ 4.15 (d, J=5.9 Hz, 2H), 4.05-3.73 (m, 2H), 3.42-3.23 (m, 1H), 3.16-2.92 (m, 4H), 2.38-2.14 (m, 2H), 1.93-1.76 (m, 1H), 1.47 (s, 9H).

¹⁹F NMR (376 MHz, Chloroform-d) δ −99.3-−101.2 (m), −102.1-−103.9 (m).

UPLC-MS (basic method, 2 min, ELSD): Rt: 1.07 min, m/z=274.1 [M−tBu+2H]⁺.

### tert-Butyl 5-[(1,3-dioxoisoindolin-2-yl)methyl]-3,3-difluoro-piperidine-1-carboxylate

A solution of tert-butyl 3,3-difluoro-5-(methylsulfonyloxymethyl)piperidine-1-carboxylate (1.25 g, 3.80 mmol) and potassium phthalimide (0.84 g, 4.55 mmol) in DMF (45 mL) was heated to 80° C. and stirred for 5 hours. The reaction mixture was then diluted with EtOAc (200 mL) and the organic layer was washed with water (3×200 mL) followed by a saturated aqueous brine solution (200 mL). The organic layer was then concentrated under reduced pressure. The crude material was purified by normal-phase column chromatography, over silica gel, eluting with a gradient of 1:4 EtOAc:iso-hexane to 4:1 EtOAc:iso-hexane to afford the title compound as a solid (1.36 g, 3.58 mmol, 94%).

¹H NMR (400 MHz, Chloroform-d) δ 7.89-7.80 (m, 2H), 7.79-7.68 (m, 2H), 4.34-4.15 (m, 1H), 3.71-3.55 (m, 2H), 2.96 (dd, J=29.5, 13.9 Hz, 1H), 2.72-2.44 (m, 1H), 2.40-2.17 (m, 2H), 1.71-1.52 (m, 2H), 1.42 (s, 9H).

¹⁹F NMR (376 MHz, Chloroform-d) δ −99.7-−101.6 (m), −103.3-−105.1 (m).

UPLC-MS (basic method, 2 min, ELSD): Rt: 1.19 min, m/z=325.1 [M−tBu+2H]⁺.

### 2-[(5,5-Difluoro-3-piperidyl)methyl]isoindoline-1,3-dione;2,2,2-trifluoroacetic acid

To a solution of tert-butyl 5-[(1,3-dioxoisoindolin-2-yl)methyl]-3,3-difluoro-piperidine-1-carboxylate (1.36 g, 3.58 mmol) in CH₂Cl₂ (15 mL) was added trifluoroacetic acid (1.4 mL, 18 mmol) and the reaction mixture was stirred at room temperature for 30 minutes. The reaction mixture was then concentrated under reduced pressure to afford the title compound as a solid (1.28 g, 3.25 mmol, 90%).

¹H NMR (400 MHz, DMSO-d₆) δ 9.50 (s, 2H), 7.91-7.82 (m, 4H), 3.71-3.56 (m, 3H), 3.40-3.22 (m, 2H), 2.85 (t, J=12.4 Hz, 1H), 2.44-2.28 (m, 2H), 2.09-1.86 (m, 1H).

¹⁹F NMR (376 MHz, DMSO-d₆) δ −74.0 (s), −97.2-−98.1 (m), −98.1-−99.0 (m).

UPLC-MS (basic method, 2 min, ELSD): Rt: 0.94 min, m/z=281.1 [M+H]⁺.

### 2-[[1-[4-[(5-Cyclopropyl-1H-pyrazol-3-yl)amino]pyrimidin-2-yl]-5,5-difluoro-3-piperidyl]methyl]isoindoline-1,3-dione

A solution of 2-chloro-N-(5-cyclopropyl-1H-pyrazol-3-yl)pyrimidin-4-amine (200 mg, 0.849 mmol), 2-[(5,5-difluoro-3-piperidyl)methyl]isoindoline-1,3-dione;2,2,2-trifluoroacetic acid (402 mg, 1.02 mmol) and N,N-diisopropylethylamine (0.52 mL, 2.97 mmol) in anhydrous NMP (3 mL) was heated to 120° C. and stirred for 18 hours. The reaction mixture was cooled to room temperature, then added dropwise to ice water (ca. 100 mL), resulting in the formation of a precipitate. The precipitate was collected by filtration, the filter cake was washed with water (2×10 mL), then dried under reduced pressure. The crude material was purified by normal-phase column chromatography, over silica gel, eluting with a gradient of CH₂Cl₂ to 9:1 CH₂Cl₂:2 M NH₃ in MeOH to afford the title compound as a gum (295 mg, 0.615 mmol, 73%).

¹H NMR (400 MHz, DMSO-d₆) δ 11.97 (s, 1H), 9.45 (s, 1H), 7.94-7.81 (m, 5H), 6.30 (s, 1H), 6.06 (s, 1H), 4.82 (t, J=12.3 Hz, 1H), 4.64 (d, J=13.2 Hz, 1H), 4.11 (d, J=5.4 Hz, 1H), 4.08 (d, J=5.3 Hz, 1H), 3.71-3.56 (m, 2H), 2.82 (t, J=12.1 Hz, 1H), 2.29-2.08 (m, 2H), 1.85 (tt, J=8.4, 5.1 Hz, 1H), 0.97-0.86 (m, 2H), 0.72-0.63 (m, 2H).

¹⁹F NMR (376 MHz, DMSO-d₆) δ −97.6-−98.8 (m), −101.4-−102.8 (m).

UPLC-MS (basic method, 2 min): Rt: 1.08 min, m/z=480.2 [M+H]⁺.

### 2-[5-(Aminomethyl)-3,3-difluoro-1-piperidyl]-N-(5-cyclopropyl-1H-pyrazol-3-yl)pyrimidin-4-amine

To a solution of 2-[[1-[4-[(5-cyclopropyl-1H-pyrazol-3-yl)amino]pyrimidin-2-yl]-5,5-difluoro-3-piperidyl]methyl]isoindoline-1,3-dione (292 mg, 0.609 mmol) in ethanol (10 mL) was added hydrazine hydrate (0.30 mL, 3.0 mmol) at room temperature. The reaction mixture was then heated to 60° C. and stirred for 16 hours. The reaction mixture was then filtered through celite, and the filtrate was concentrated to dryness under reduced pressure. The crude residue was dissolved in MeOH, then loaded onto a SCX-2 cartridge. The cartridge was washed with MeOH (20 mL), then the compound was eluted with 2 M NH₃ in MeOH (20 mL). The eluent was concentrated to dryness under reduced pressure. The residue was purified by reversed-phase basic prepHPLC to afford the title compound as a solid. (60 mg, 0.17 mmol, 28%).

¹H NMR (400 MHz, Methanol-d₄) δ 7.88 (d, J=5.9 Hz, 1H), 6.22 (d, J=5.9 Hz, 1H), 6.06 (s, 1H), 4.68-4.56 (m, 1H), 4.50-4.42 (m, 1H), 3.55-3.41 (m, 1H), 3.04 (dd, J=13.4, 9.5 Hz, 1H), 2.73-2.65 (m, 1H), 2.60 (dd, J=13.0, 6.3 Hz, 1H), 2.37-2.21 (m, 1H), 2.01-1.86 (m, 2H), 1.85-1.67 (m, 1H), 1.03-0.92 (m, 2H), 0.77-0.67 (m, 2H).

¹⁹F NMR (376 MHz, Methanol-d₄) δ−100.0-−100.8 (m), −103.2-−104.1 (m).

UPLC-MS (basic method, 4 min): Rt: 1.20 min, m/z=350.3 [M+H]⁺.

### Example 105: Synthesis of Compound 177

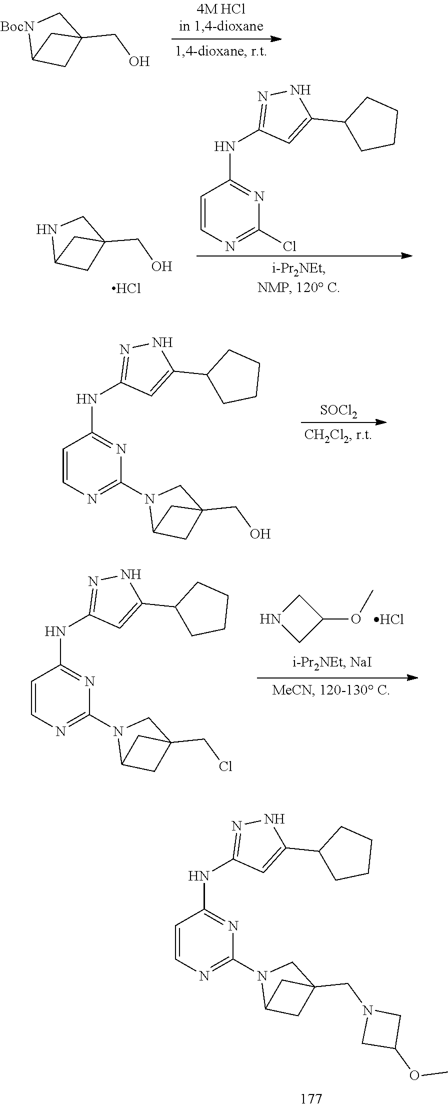

### N-(5-Cyclopentyl-1H-pyrazol-3-yl)-2-[4-[(3-methoxyazetidin-1-yl)methyl]-2-azabicyclo[2.1.1]hexan-2-yl]pyrimidin-4-amine

A suspension of 2-[4-(chloromethyl)-2-azabicyclo[2.1.1]hexan-2-yl]-N-(5-cyclopentyl-1H-pyrazol-3-yl)pyrimidin-4-amine (150 mg, 0.418 mmol), 3-methoxyazetidine hydrochloride (517 mg, 4.18 mmol), N,N-diisopropylethylamine (0.95 mL, 5.4 mmol) and sodium iodide (63 mg, 0.42 mmol) in MeCN (3 mL) was heated to 120° C. in a sealed tube, and stirred for 16 hours. The reaction mixture was then heated to 130° C. and stirred for a further 6 hours. The reaction mixture was diluted with a 0.5 M aqueous NaOH solution (10 mL), then extracted with 1:3 IPA:CHCl3 (3×10 mL). The combined organic extracts were dried over Na₂SO₄, then concentrated to dryness under reduced pressure. The crude residue was purified by reversed-phase basic prepHPLC to afford a colorless solid. The residue was then loaded onto an SCX-2 cartridge. The cartridge was washed with MeOH (25 mL), then the compound was eluted with a 2 M solution of NH₃ in MeOH (25 mL). The eluent was concentrated to dryness under reduced pressure to afford the title compound as a solid (12 mg, 29 μmol, 7%).

1H NMR (400 MHz, Chloroform-d) δ 8.00 (d, J=5.7 Hz, 1H), 6.80 (s, 1H), 6.12 (d, J=5.7 Hz, 1H), 6.03 (s, 1H), 4.82 (s, 1H), 4.05 (p, J=5.9 Hz, 1H), 3.69 (t, J=6.8 Hz, 2H), 3.45 (s, 2H), 3.25 (s, 3H), 3.05 (p, J=8.1 Hz, 1H), 2.99-2.92 (m, 2H), 2.80 (s, 2H), 2.16-2.02 (m, 2H), 1.89-1.61 (m, 8H), 1.58-1.49 (m, 2H).

UPLC-MS (basic method, 4 min): Rt: 1.54 min, m/z=410.4 [M+H]+.

### Example 106: Synthesis of Compound 119

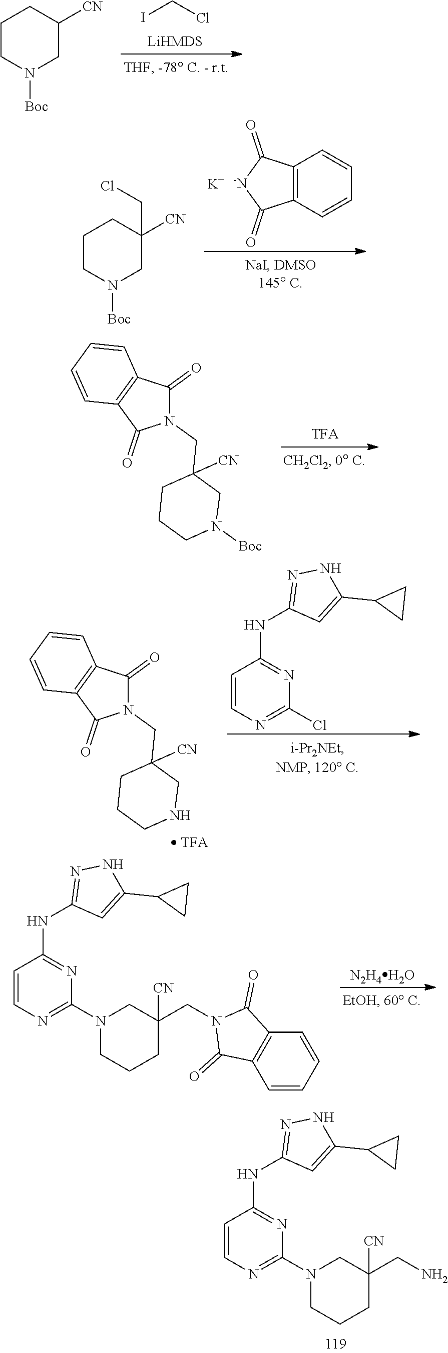

### tert-Butyl 3-(chloromethyl)-3-cyano-piperidine-1-carboxylate

To a solution of 1-boc-piperidine-3-carbonitrile (2.00 g, 9.51 mmol) in THF (20 mL) was added a 1.0 M solution of lithium bis(trimethylsilyl)amide THF (11 mL, 10.5 mmol) at −78° C. and the reaction mixture was stirred for one hour. Chloroiodomethane (1.76 g, 9.99 mmol) in THF (10 mL) was added at −78° C. then reaction mixture was warmed to room temperature and stirred for 16 hours. The reaction was then quenched with a saturated aqueous ammonium chloride solution (5 mL) and stirred for 15 min. Reaction mixture was concentrated to dryness under reduced pressure, diluted with EtOAc (20 mL). The organic phase was washed with water (10 mL) dried over Na₂SO₄ then concentrated to dryness under reduced pressure to the title compound as a solid (2.10 g, 8.12 mmol, 85%).

1H NMR (400 MHz, Chloroform-d) δ 4.21 (s, 1H), 3.90 (s, 1H), 3.57 (d, J=4.7 Hz, 2H), 3.05 (s, 1H), 2.99-2.88 (m, 1H), 2.18-2.07 (m, 1H), 1.89-1.75 (m, 1H), 1.76-1.61 (m, 2H), 1.47 (s, 9H).

UPLC-MS (basic method, 2 min, ELSD) Rt: 1.07 min, m/z=259.1 [M+H]+.

### tert-Butyl 3-Cyano-3-[(1,3-dioxoisoindolin-2-yl)methyl]piperidine-1-carboxylate

To a stirred solution of tert-butyl 3-(chloromethyl)-3-cyano-piperidine-1-carboxylate (550 mg, 2.13 mmol) in DMSO (5 mL) was added potassium phthalimide (433 mg, 2.34 mmol) and sodium iodide (478 mg, 3.19 mmol). The reaction mixture was heated to 145° C. and stirred for 16 hours. The reaction mixture was then cooled to room temperature, diluted with water (10 mL) and extracted with ethyl acetate (3×10 mL). The combined organic extracts were washed with a saturated aqueous brine solution (6×10 mL), dried over Na₂SO₄, and concentrated to dryness under reduced pressure. The crude residue was purified by normal-phase flash column chromatography, over silica gel, eluting with 1:1 EtOAc:iso-hexane to afford the title compound as a solid (180 mg, 0.487 mmol, 23%).

1H NMR (400 MHz, Chloroform-d) δ 8.14-7.81 (m, 2H), 7.83-7.67 (m, 2H), 4.31 (s, 1H), 3.84 (d, J=11.4 Hz, 2H), 3.24-2.59 (m, 2H), 2.24-2.05 (m, 1H), 1.94-1.66 (m, 2H), 1.63-1.51 (m, 1H), 1.45 (d, J=0.8 Hz, 1H), 1.41 (s, 9H).

UPLC-MS (basic method, 2 min, ELSD) Rt: 1.13 min, m/z=370.1 [M+H]+.

### 1-[4-[(5-Cyclopropyl-1H-pyrazol-3-yl)amino]pyrimidin-2-yl]-3-[(1,3-dioxoisoindolin-2-yl)methyl]piperidine-3-carbonitrile

A solution of 2-chloro-N-(5-cyclopropyl-1H-pyrazol-3-yl)pyrimidin-4-amine (65 mg, 0.28 mmol), 3-[(1,3-dioxoisoindolin-2-yl)methyl]piperidine-3-carbonitrile;2,2,2-trifluoroacetic acid (127 mg, 0.331 mmol) and N,N-diisopropylethylamine (0.19 mL, 1.1 mmol) in NMP (1 mL) was heated to 130° C. and stirred for 48 hours. The reaction mixture was then cooled to room temperature, poured into ice water, and stirred for 30 minutes. Solid was collected by filtration, and the filter cake was washed with iso-hexanes. The crude material was purified by normal-phase flash column chromatography, over silica gel, eluting with 49:1 CH2Cl2:MeOH to afford the title compound as a solid (110 mg, 0.141 mmol).

1H NMR (400 MHz, Chloroform-d) δ 7.93 (d, J=5.7 Hz, 1H), 7.91-7.82 (m, 2H), 7.78-7.69 (m, 2H), 7.36 (s, 1H), 6.18 (d, J=5.8 Hz, 1H), 5.90 (s, 1H), 4.94 (d, J=13.4 Hz, 1H), 4.50 (d, J=13.4 Hz, 1H), 4.13-3.78 (m, 2H), 3.19 (d, J=13.5 Hz, 1H), 3.08-2.97 (m, 1H), 2.89-2.80 (m, 1H), 2.19-2.12 (m, 1H), 1.97-1.78 (m, 3H), 0.99-0.90 (m, 2H), 0.84-0.65 (m, 2H).

UPLC-MS (basic method, 2 min) Rt: 1.00 min, m/z=469.3 [M+H]+.

### 3-(Aminomethyl)-1-[4-[(5-cyclopropyl-1H-pyrazol-3-yl)amino]pyrimidin-2-yl]piperidine-3-carbonitrile

To a stirred solution of 1-[4-[(5-cyclopropyl-1H-pyrazol-3-yl)amino]pyrimidin-2-yl]-3-[(1,3-dioxoisoindolin-2-yl)methyl]piperidine-3-carbonitrile (140 mg, 0.179 mmol) in ethanol (2 mL) was added hydrazine hydrate (87 μL, 0.90 mmol). The reaction mixture was heated to 60° C. and stirred for 2 hours. Reaction mixture was then cooled to room temperature and then filtered through celite. The filtrate was concentrated to dryness under reduced pressure. The crude residue was dissolved in MeOH, then loaded onto an SCX-2 cartridge. The cartridge was washed with MeOH (20 mL), then the compound was eluted with 2 M NH₃ in MeOH (20 mL). The eluent was concentrated to dryness under reduced pressure. The residue was then purified by reversed-phase basic prepHPLC to afford the title compound as a solid (28 mg, 80 μmol, 45%).

1H NMR (400 MHz, Chloroform-d) δ 7.98 (d, J=5.7 Hz, 1H), 7.67 (s, 1H), 6.17 (d, J=5.8 Hz, 1H), 5.90 (s, 1H), 4.20 (d, J=13.4 Hz, 1H), 3.86 (d, J=13.5 Hz, 2H), 3.75 (s, 1H), 2.87 (dd, 2H), 2.20-2.03 (m, 1H), 1.86 (td, J=8.5, 4.4 Hz, 1H), 1.74 (d, J=10.6 Hz, 4H), 1.09-0.87 (m, 2H), 0.86-0.65 (m, 2H).

UPLC-MS (basic method, 4 min) Rt: 1.17 min, m/z=339.2 [M+H]+.

### Example 107: Synthesis of Compound 161

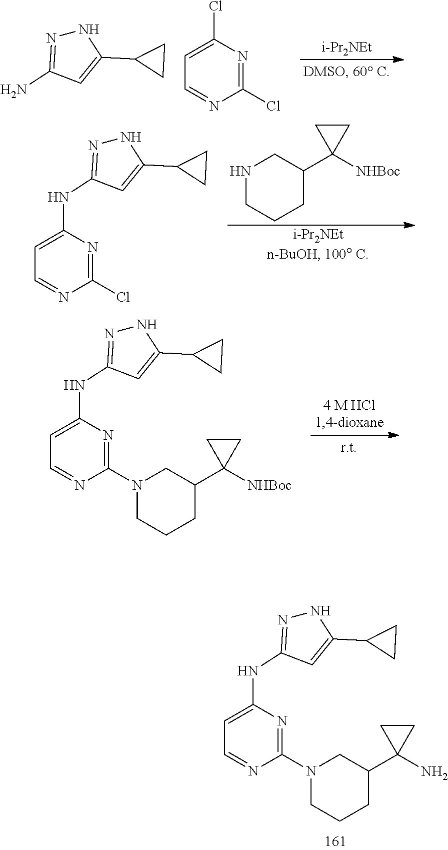

### tert-Butyl N-[1-[1-[4-[(5-cyclopropyl-1H-pyrazol-3-yl)amino]pyrimidin-2-yl]-3-piperidyl]-1-methyl-propyl]carbamate

To a solution of 2-chloro-N-(5-cyclopropyl-1H-pyrazol-3-yl)pyrimidin-4-amine (404 mg, 1.71 mmol) and tert-butyl N-(1-piperidin-3-ylcyclopropyl)carbamate (474 mg, 1.97 mmol) in n-butanol (17 mL) was added N,N-diisopropylethylamine (0.90 mL, 5.1 mmol). The resulting solution was then heated to 100° C. and stirred for 16 h. The reaction mixture was concentrated under reduced pressure and diluted with water (40 mL). The resulting solution was extracted with DCM (3×20 mL). The combined organic fractions were dried over Na₂SO₄ and concentrated to dryness under reduced pressure to afford the title compound as a solid (0.77 g, 1.59 mmol, 93%).

1H NMR (400 MHz, DMSO-d₆) δ 11.90 (s, 1H), 9.31 (s, 1H), 7.82 (d, J=5.7 Hz, 1H), 7.12 (s, 1H), 6.17 (s, 2H), 4.68 (dd, J=50.4, 12.9 Hz, 2H), 4.31 (t, J=5.2 Hz, 1H), 3.42-3.33 (m, 2H), 2.66-2.53 (m, 1H), 1.93-1.60 (m, 3H), 1.43-1.37 (m, 3H), 1.37 (s, 9H), 1.34-1.24 (m, 3H), 1.04 (d, J=6.1 Hz, 1H), 0.95-0.90 (m, 1H), 0.61 (d, J=5.4 Hz, 1H).

UPLC-MS (basic method, 4 min): R.t.: 1.11 min, m z: 440.5 [M+H]⁺.

### 2-[3-(1-Aminocyclopropyl)-1-piperidyl]-N-(5-cyclopropyl-1H-pyrazol-3-yl)pyrimidin-4-amine

To tert-butyl N-[1-[1-[4-[(5-cyclopropyl-1H-pyrazol-3-yl)amino]pyrimidin-2-yl]-3-piperidyl]cyclopropyl]carbamate (104 mg, 0.237 mmol) was added 4 M HCl in 1,4-dioxane (0.59 mL, 2.4 mmol). The resulting mixture was stirred at room temperature for 2 h. The volatiles were removed under reduced pressure to afford a yellow solid, which was dissolved in methanol (2 mL) and loaded onto an SCX cartridge (5 g). The cartridge was washed with MeOH (20 mL) then the compound was eluted with 2 M NH₃ in MeOH (20 mL). The volatiles were removed under reduced pressure to afford the title compound as a powder (60 mg, 0.17 mmol, 72%).

¹H NMR (400 MHz, DMSO-d₆) δ 11.91 (s, 1H), 9.31 (s, 1H), 7.83 (d, J=5.7 Hz, 1H), 6.17 (s, 2H), 4.65 (d, J=12.7 Hz, 2H), 2.80-2.70 (m, 1H), 2.67-2.56 (m, 1H), 1.91-1.80 (m, 1H), 1.78-1.64 (m, 3H), 1.60-1.44 (m, 1H), 1.37-1.29 (m, 1H), 0.95-0.89 (m, 2H), 0.86-0.74 (m, 1H), 0.71-0.65 (m, 2H), 0.52-0.43 (m, 1H), 0.41-0.27 (m, 3H).

UPLC-MS (basic method, 4 min): R.t.: 1.36 min, m z: 340.3 [M+H]⁺.

### Example 108: Synthesis of Compound 159-1 and 159-2

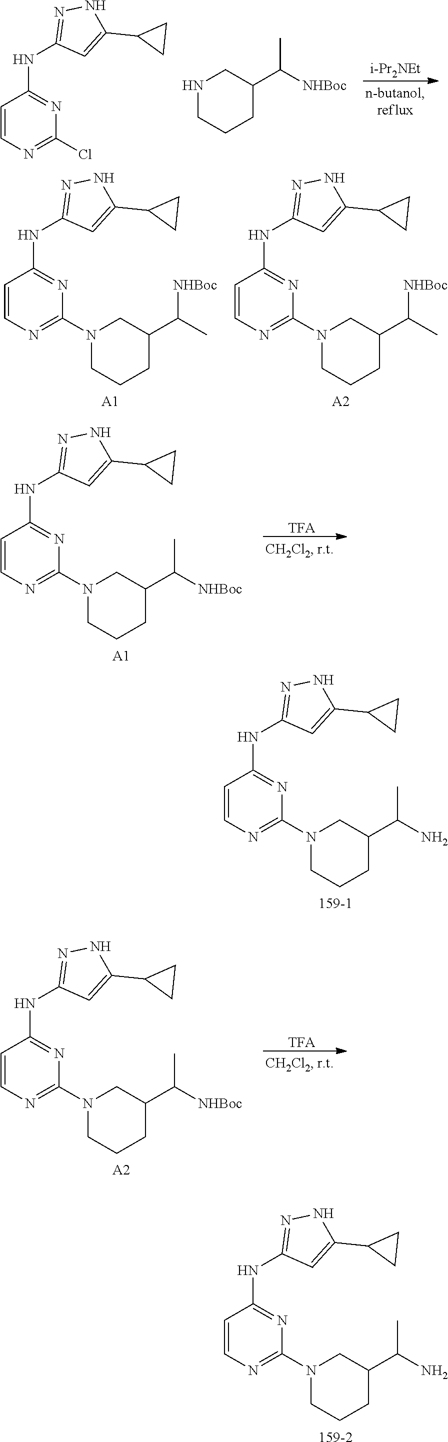

### tert-Butyl N-[1-[1-[4-[(5-Cyclopropyl-1H-pyrazol-3-yl)amino]pyrimidin-2-yl]-3-piperidyl]ethyl]carbamate

To a stirred solution of 2-chloro-N-(5-cyclopropyl-1H-pyrazol-3-yl)pyrimidin-4-amine (250 mg, 1.06 mmol) and tert-butyl N-(1-(piperidin-3-yl)ethyl)carbamate (363 mg, 1.59 mmol) in n-butanol (3 mL) was added N,N-diisopropylethylamine (462 μL, 2.65 mmol). The reaction mixture was heated to 120° C. and stirred overnight, then concentrated to dryness under reduced pressure. The residue was purified by reversed-phase basic prep-HPLC to afford two diastereomers of the title compound. Diastereomer A1 was obtained as a solid (190 mg, 0.444 mmol, 42%). Diastereomer A2 was obtained as a solid (196 mg, 0.458 mmol, 43%).

### A1:

1H NMR (400 MHz, Chloroform-d) δ 10.83 (s, 1H), 7.92 (d, J=5.7 Hz, 1H), 6.05-6.00 (m, 2H), 4.68-4.49 (m, 2H), 3.64-3.54 (m, 1H), 2.85-2.73 (m, 1H), 2.69-2.57 (m, 1H), 1.99-1.82 (m, 1H), 1.86-1.75 (m, 1H), 1.76-1.68 (m, 1H), 1.52-1.36 (m, 12H), 1.31-1.17 (m, 1H), 1.14 (d, J=6.8 Hz, 3H), 0.95-0.83 (m, 2H), 0.72-0.61 (m, 2H).

### A2:

¹H NMR (400 MHz, Chloroform-d) δ 7.92 (d, J=5.7 Hz, 1H), 7.70 (d, J=5.7 Hz, 1H), 6.56-5.54 (m, 2H), 4.96-4.45 (m, 3H), 3.58 (d, J=10.0 Hz, 1H), 2.83-2.72 (m, 1H), 2.71-2.50 (m, 1H), 1.97-1.76 (m, 2H), 1.78-1.65 (m, 1H), 1.54-1.37 (m, 11H), 1.24 (d, J=14.1 Hz, 1H), 1.11 (d, J=6.7 Hz, 3H), 0.94-0.82 (m, 2H), 0.72-0.64 (m, 2H).

### 2-[3-(1-Aminoethyl)-1-piperidyl]-N-(5-cyclopropyl-1H-pyrazol-3-yl)pyrimidin-4-amine (Compound 159-1)

To a stirred solution of tert-butyl N-[1-[1-[4-[(5-cyclopropyl-1H-pyrazol-3-yl)amino]pyrimidin-2-yl]-3-piperidyl]ethyl]carbamate (diastereomer 1) (69 mg, 0.16 mmol) in DCM (3 mL) was added trifluoroacetic acid (2.0 mL, 0.161 mmol) at room temperature. The reaction mixture was stirred for 30 minutes. The volatiles were removed under reduced pressure. The residue was dissolved in methanol and loaded onto an SCX-2 cartridge. The cartridge was washed with methanol, then the compound was eluted with 2 M NH₃ in methanol. The eluent was concentrated to dryness under reduced pressure to afford the title compound as a solid (47 mg, 0.144 mmol, 89%).

1H NMR (400 MHz, Chloroform-d) δ 7.89 (dd, J=5.7, 0.7 Hz, 1H), 6.01 (d, J=5.7 Hz, 1H), 5.96-5.92 (m, 1H), 4.73-4.36 (m, 2H), 2.86-2.73 (m, 2H), 2.63 (dd, J=12.9, 10.7 Hz, 1H), 1.95-1.86 (m, 1H), 1.87-1.66 (m, 2H), 1.55-1.31 (m, 2H), 1.31-1.16 (m, 1H), 1.12 (d, J=6.5 Hz, 3H), 0.96-0.81 (m, 2H), 0.75-0.63 (m, 2H).

UPLC-MS (4 min, basic method) R.t.: 1.27 min, m/z=328.2 [M+H]+.

### 2-[3-(1-Aminoethyl)-1-piperidyl]-N-(5-cyclopropyl-1H-pyrazol-3-yl)pyrimidin-4-amine (Compound 159-2)

To a stirred solution of tert-butyl N-[1-[1-[4-[(5-cyclopropyl-1H-pyrazol-3-yl)amino]pyrimidin-2-yl]-3-piperidyl]ethyl]carbamate (diastereomer 2) (63 mg, 0.15 mmol) in DCM (3 mL) was added trifluoroacetic acid (2.0 mL, 0.147 mmol) at room temperature. The reaction mixture was stirred for 30 minutes. The volatiles were removed under reduced pressure. The residue was dissolved in methanol and loaded onto an SCX-2 cartridge. The cartridge was washed with methanol, then the compound was eluted with 2 M NH₃ in methanol. The eluent was concentrated to dryness under reduced pressure to afford the title compound as a solid (42 mg, 0.128 mmol, 87%).

1H NMR (400 MHz, Chloroform-d) δ 8.27 (s, 1H), 7.98-7.87 (m, 1H), 6.06-5.99 (m, 1H), 5.85 (s, 1H), 4.18 (d, J=13.1 Hz, 1H), 3.98 (d, J=12.7 Hz, 1H), 3.53-3.37 (m, 2H), 2.89-2.76 (m, 1H), 2.05-1.75 (m, 2H), 1.74-1.64 (m, 1H), 1.60-1.30 (m, 3H), 1.10 (d, J=1.6 Hz, 3H), 0.95-0.85 (m, 2H), 0.76-0.65 (m, 2H).

UPLC-MS (4 min, basic method) R.t.: 1.38 min, m/z=328.2 [M+H]+.

### Example 109: Synthesis of Compound 162-1 and 162-2

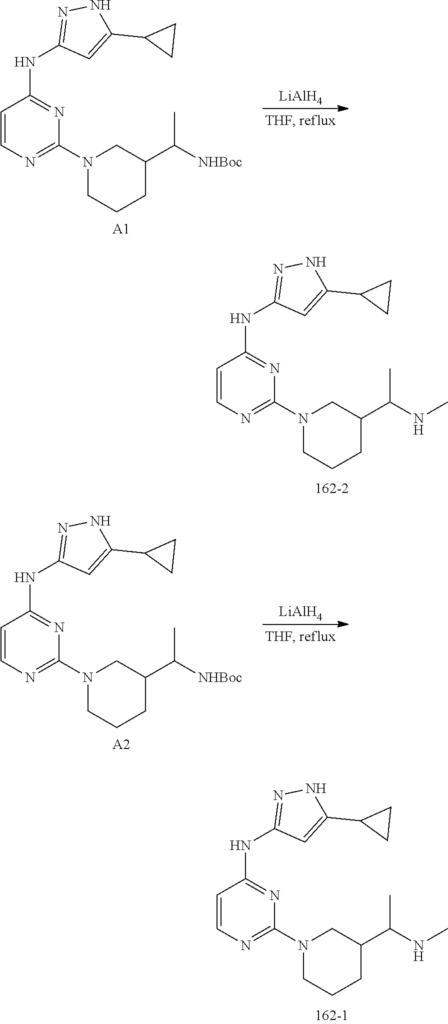

### N-(5-Cyclopropyl-1H-pyrazol-3-yl)-2-[3-[1-(methylamino)ethyl]-1-piperidyl]pyrimidin-4-amine (Compound 162-2)

To a solution of tert-butyl N-[1-[1-[4-[(5-cyclopropyl-1H-pyrazol-3-yl)amino]pyrimidin-2-yl]-3-piperidyl]ethyl]carbamate (Diastereomer A1) (90 mg, 0.211 mmol) in THF (5 mL) was added a 2.4 M solution of lithium aluminum hydride in THF (880 μL, 2.11 mmol) at 0° C. The reaction mixture was heated to reflux and stirred for 4 hours. Reaction mixture was cooled to room temperature and quenched with water (100 μl) followed by a 10% w/v aqueous solution of NaOH (10%, 400 μl). After 15 minutes of stirring, MgSO₄ (500 mg) was added, and reaction mixture was left for stirring for 16 hours.

Reaction mixture was filtered through celite, washed with dichloromethane (50 mL) followed by methanol (20 mL). Volatiles were removed under reduced pressure. The residue was dissolved in methanol, then loaded onto an SCX-2 cartridge. The cartridge was washed with MeOH, then the compound was eluted with 2 M NH₃ in MeOH. The eluent was concentrated to dryness under reduced pressure. The residue was purified by flash column chromatography, eluting with 1:1 DCM:MeOH to afford the title compound as a solid (33 mg, 97 μmol, 46%).

1H NMR (400 MHz, Chloroform-d) δ 8.20 (s, 1H), 7.93 (d, J=5.7 Hz, 1H), 6.05 (d, J=5.7 Hz, 1H), 5.91 (s, 1H), 4.76-4.67 (m, 1H), 4.59 (d, J=13.1 Hz, 1H), 2.85-2.74 (m, 1H), 2.65 (dd, J=12.9, 11.0 Hz, 1H), 2.60-2.48 (m, 1H), 2.43 (s, 3H), 1.90-1.75 (m, 2H), 1.76-1.68 (m, 1H), 1.66-1.53 (m, 1H), 1.53-1.41 (m, 1H), 1.41-1.27 (m, 1H), 1.09 (d, J=6.5 Hz, 3H), 0.97-0.84 (m, 2H), 0.74-0.64 (m, 2H).

UPLC MS (4 min, basic method), rt=1.42 min, m/z=342.2 [M+H]+.

### N-(5-Cyclopropyl-1H-pyrazol-3-yl)-2-[3-[1-(methylamino)ethyl]-1-piperidyl]pyrimidin-4-amine (Compound 162-1)

To a solution of tert-butyl N-[1-[1-[4-[(5-cyclopropyl-1H-pyrazol-3-yl)amino]pyrimidin-2-yl]-3-piperidyl]ethyl]carbamate (Diastereomer A2) (90 mg, 0.211 mmol) in THE (5 mL) was added a 2.4 M solution of lithium aluminum hydride in THE (880 μL, 2.11 mmol) at 0° C. The reaction mixture was heated to reflux and stirred for 4 hours. Reaction mixture was cooled to room temperature and quenched with water (100 μl) followed by a 10% w/v aqueous solution of NaOH (10%, 400 μl). After 15 minutes of stirring, MgSO₄ (500 mg) was added, and reaction mixture was left for stirring for 16 hours.

Reaction mixture was filtered through celite, washed with dichloromethane (50 mL) followed by methanol (20 mL). Volatiles were removed under reduced pressure. The residue was dissolved in methanol, then loaded onto an SCX-2 cartridge. The cartridge was washed with MeOH, then the compound was eluted with 2 M NH₃ in MeOH. The eluent was concentrated to dryness under reduced pressure. The residue was purified by flash column chromatography, eluting with 1:1 DCM:MeOH to afford the title compound as a solid (33 mg, 97 μmol, 46%).

¹H NMR (400 MHz, Chloroform-d) δ 8.20 (s, 1H), 7.93 (d, J=5.7 Hz, 1H), 6.05 (d, J=5.7 Hz, 1H), 5.91 (s, 1H), 4.76-4.67 (m, 1H), 4.59 (d, J=13.1 Hz, 1H), 2.85-2.74 (m, 1H), 2.65 (dd, J=12.9, 11.0 Hz, 1H), 2.60-2.48 (m, 1H), 2.43 (s, 3H), 1.90-1.75 (m, 2H), 1.76-1.68 (m, 1H), 1.66-1.53 (m, 1H), 1.53-1.41 (m, 1H), 1.41-1.27 (m, 1H), 1.09 (d, J=6.5 Hz, 3H), 0.97-0.84 (m, 2H), 0.74-0.64 (m, 2H).

UPLC MS (4 min, basic method), rt=1.42 min, m/z=342.2 [M+H]⁺.

### Example 110: Synthesis of Compound 187

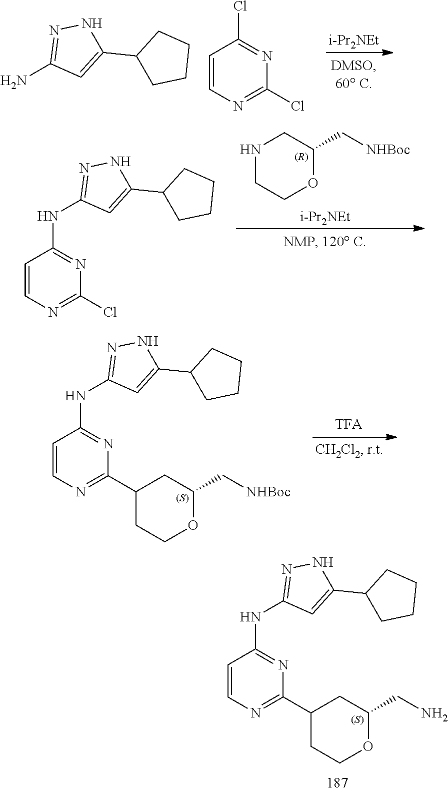

### tert-Butyl N-[[(2S)-4-[4-[(5-Cyclopentyl-1H-pyrazol-3-yl)amino]pyrimidin-2-yl]morpholin-2-yl]methyl]carbamate

A solution of 2-chloro-N-(5-cyclopentyl-1H-pyrazol-3-yl)pyrimidin-4-amine (254 mg, 0.963 mmol), (R)-2-(Boc-aminomethyl)morpholine (250 mg, 1.16 mmol) and N,N-diisopropylethylamine (0.42 mL, 2.4 mmol) in NMP (3 mL) was heated to 120° C. and stirred for 16 hours. The reaction mixture was then cooled to room temperature, then added dropwise to ice water (ca. 100 mL) resulting in the formation of a precipitate. The precipitate was collected by filtration, then the filter cake was washed with water (2×10 mL), then dried under reduced pressure to afford the title compound as a solid (331 mg, 0.746 mmol, 77%).

¹H NMR (400 MHz, DMSO-d₆) δ 11.94 (s, 1H), 9.41 (s, 1H), 7.89 (d, J=5.7 Hz, 1H), 6.91 (t, J=5.9 Hz, 1H), 6.34 (s, 1H), 6.18 (s, 1H), 4.45 (d, J=13.1 Hz, 1H), 4.33 (d, J=13.1 Hz, 1H), 3.94-3.84 (m, 1H), 3.49-3.36 (m, 2H), 3.14-2.85 (m, 4H), 2.63-2.53 (m, 1H), 2.02-1.93 (m, 2H), 1.77-1.50 (m, 6H), 1.38 (s, 9H).

UPLC-MS (basic method, 2 min): Rt: 1.11 min, m/z: 444.3 [M+H]⁺.

### 2-[(2S)-2-(Aminomethyl)morpholin-4-yl]-N-(5-cyclopentyl-1H-pyrazol-3-yl)pyrimidin-4-amine

To a solution of tert-butyl N-[[(2S)-4-[4-[(5-cyclopentyl-1H-pyrazol-3-yl)amino]pyrimidin-2-yl]morpholin-2-yl]methyl]carbamate (100 mg, 0.225 mmol) in CH₂Cl₂ (5 mL) was added trifluoroacetic acid (5.0 mL, 65 mmol). The reaction mixture was stirred at room temperature for 3 hours. The reaction mixture was concentrated to dryness under reduced pressure. The residue was dissolved in MeOH (2 mL) then loaded onto a SCX-2 cartridge. The cartridge was washed with MeOH (25 mL) then the compound was eluted with 2 M NH₃ in MeOH (25 mL). The eluent was concentrated to dryness under reduced pressure to afford the title compound as a solid (60 mg, 0.18 mmol, 77%).

¹H NMR (400 MHz, DMSO-d₆) δ 11.93 (s, 1H), 9.42 (s, 1H), 7.88 (d, J=5.6 Hz, 1H), 6.49-5.98 (m, 2H), 4.54-4.46 (m, 1H), 4.41-4.30 (m, 1H), 3.90 (dd, J=11.2, 3.2 Hz, 1H), 3.46 (td, J=11.5, 2.8 Hz, 1H), 3.00 (p, J=7.9 Hz, 1H), 2.89 (td, J=12.4, 3.5 Hz, 1H), 2.70-2.55 (m, 3H), 2.06-1.92 (m, 2H), 1.77-1.48 (m, 7H).

UPLC-MS (basic method, 4 min): rt=1.27 min, m/z=344.3 [M+H]⁺.

### Example 111: Synthesis of Compound 188

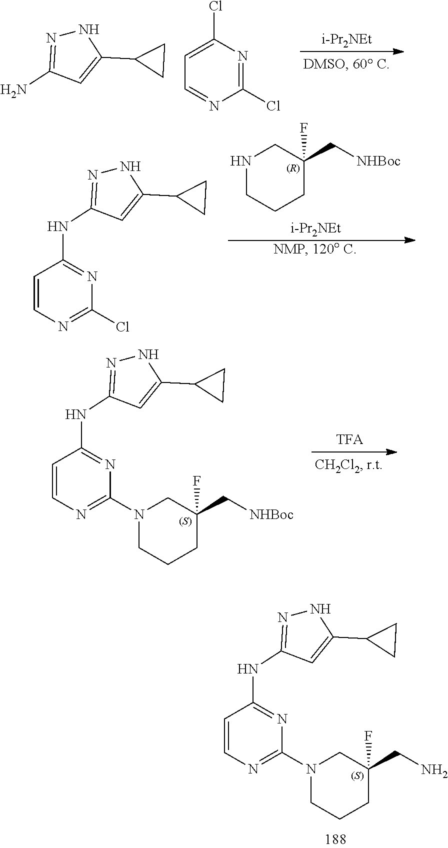

### tert-Butyl N-[[(3S)-1-[4-[(5-Cyclopropyl-1H-pyrazol-3-yl)amino]pyrimidin-2-yl]-3-fluoro-3-piperidyl]methyl]carbamate

A solution of 2-chloro-N-(5-cyclopropyl-1H-pyrazol-3-yl)pyrimidin-4-amine (210 mg, 0.891 mmol), tert-butyl (R)-((3-fluoropiperidin-3-yl)methyl)carbamate (245 mg, 1.05 mmol) and N,N-diisopropylethyl amine (0.55 mL, 3.2 mmol) in NMP (4 mL) was heated to 120° C. and stirred for 20 hours. The reaction mixture was then cooled to room temperature, then added dropwise to ice water (ca. 100 mL) resulting in the formation of a precipitate. The precipitate was collected by filtration, then the filter cake was washed with water (2×10 mL) then dried under reduced pressure to the title compound as a solid (353 mg, 0.785 mmol, 74%).

¹H NMR (400 MHz, DMSO-d₆) δ 11.94 (s, 1H), 9.32 (s, 1H), 7.85 (d, J=5.7 Hz, 1H), 7.09 (t, J=6.4 Hz, 1H), 6.26 (s, 1H), 6.07 (s, 1H), 4.43 (t, J=12.0 Hz, 1H), 4.33 (d, J=13.0 Hz, 1H), 3.31-3.00 (m, 2H), 1.88 (m, 1H), 1.78 (s, 2H), 1.60 (s, 3H), 1.39 (s, 9H), 0.98-0.85 (m, 3H), 0.67 (m, 2H).

UPLC-MS (basic method, 4 min): Rt: 1.72 min, m/z: 432.3 [M+H]⁺.

### 2-[(3S)-3-(Aminomethyl)-3-fluoro-1-piperidyl]-N-(5-cyclopropyl-1H-pyrazol-3-yl)pyrimidin-4-amine

To a solution of tert-butyl tert-butyl N-[[(3S)-1-[4-[(5-cyclopropyl-1H-pyrazol-3-yl)amino]pyrimidin-2-yl]-3-fluoro-3-piperidyl]methyl]carbamate (339 mg, 0.785 mmol) in CH₂Cl₂ (5 mL) was added trifluoroacetic acid (1.2 mL, 16 mmol) then the reaction mixture was stirred at room temperature for 1 hour. The reaction mixture was then concentrated to dryness under reduced pressure. The residue was then loaded onto a SCX-2 cartridge. The cartridge was washed with MeOH (25 mL) then the compound was eluted with 2 M solution of NH₃ in MeOH (25 mL). The eluent was concentrated to dryness under reduced pressure to afford the title compound as a solid (226 mg, 0.668 mmol, 85%).

¹H NMR (400 MHz, DMSO-d₆) δ 12.00 (s, 1H), 9.39 (s, 1H), 7.89 (d, J=5.6 Hz, 1H), 6.15 (s, 1H), 6.10 (s, 1H), 4.47-4.37 (m, 1H), 4.33-4.24 (m, 1H), 3.45-3.30 (m, 1H), 3.23 (d, J=10.5 Hz, 1H), 2.79-2.66 (m, 2H), 1.95-1.64 (m, 4H), 1.63-1.53 (m, 1H), 1.01-0.87 (m, 2H), 0.75-0.63 (m, 2H).

UPLC-MS (basic method, 4 min): rt=1.22 min, m/z=332.3 [M+H]⁺.

### Example 112: Synthesis of Compound 189

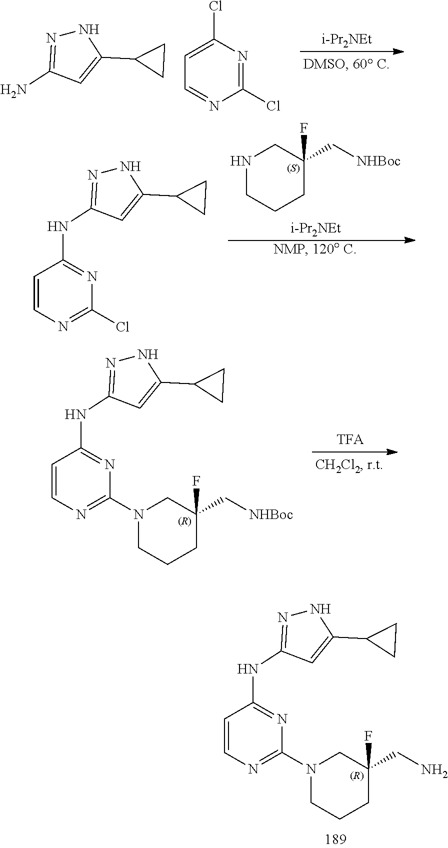

### tert-Butyl N-[[(3R)-1-[4-[(5-Cyclopropyl-1H-pyrazol-3-yl)amino]pyrimidin-2-yl]-3-fluoro-3-piperidyl]methyl]carbamate

A solution of 2-chloro-N-(5-cyclopropyl-1H-pyrazol-3-yl)pyrimidin-4-amine (210 mg, 0.891 mmol), tert-butyl N-[[(3S)-3-fluoropiperidin-3-yl]methyl]carbamate (248 mg, 1.07 mmol) and N,N-diisopropylethylamine (0.39 mL, 2.2 mmol) in NMP (3 mL) was heated to 120° C. and stirred for 18 hours. The reaction mixture was then cooled to room temperature, then added dropwise to ice water (ca. 100 mL) resulting in the formation of a precipitate. The precipitate was collected by filtration, then the filter cake was washed with water (2×10 mL) then dried under reduced pressure to the title compound as a solid (378 mg, 0.876 mmol, 98%).

¹H NMR (400 MHz, DMSO-d₆) δ 11.94 (s, 1H), 9.33 (s, 1H), 7.85 (d, J=5.7 Hz, 1H), 7.09 (t, J=6.4 Hz, 1H), 6.26 (s, 1H), 6.07 (s, 1H), 4.43 (t, J=12.0 Hz, 1H), 4.33 (d, J=13.2 Hz, 1H), 3.30-3.00 (m, 4H), 1.95-1.51 (m, 5H), 1.39 (s, 9H), 1.02-0.82 (m, 2H), 0.72-0.56 (m, 2H).

¹⁹F NMR (376 MHz, DMSO-d₆) δ −160.2-−161.9 (m).

UPLC-MS (basic method, 2 min): Rt: 1.06 min, m/z: 432.4 [M+H]⁺.

### 2-[(3R)-3-(Aminomethyl)-3-fluoro-1-piperidyl]-N-(5-cyclopropyl-1H-pyrazol-3-yl)pyrimidin-4-amine

To a solution of tert-butyl N-[[(3R)-1-[4-[(5-cyclopropyl-1H-pyrazol-3-yl)amino]pyrimidin-2-yl]-3-fluoro-3-piperidyl]methyl]carbamate (100 mg, 0.232 mmol) in CH₂Cl₂ (5 mL) was added trifluoroacetic acid (5.0 mL, 65 mmol) then the reaction mixture was stirred at room temperature for 2 hours. The reaction mixture was then concentrated to dryness under reduced pressure. The residue was then loaded onto a SCX-2 cartridge. The cartridge was washed with MeOH (25 mL) then the compound was eluted with 2 M solution of NH₃ in MeOH (25 mL). The eluent was concentrated to dryness under reduced pressure to afford the title compound as a solid (65 mg, 0.20 mmol, 85%).

¹H NMR (400 MHz, DMSO-d₆) δ 11.94 (s, 1H), 9.33 (s, 1H), 7.85 (d, J=5.7 Hz, 1H), 6.21 (s, 1H), 6.10 (s, 1H), 4.38 (t, J=11.8 Hz, 1H), 4.24 (d, J=13.1 Hz, 1H), 3.18 (t, J=10.9 Hz, 1H), 2.69 (d, J=20.5 Hz, 2H), 1.93-1.46 (m, 6H), 0.96-0.82 (m, 2H), 0.70-0.61 (m, 2H).

¹⁹F NMR (376 MHz, DMSO-d₆) δ −162.1-−162.8 (m).

UPLC-MS (basic method, 4 min): rt=1.20 min, m/z=332.3 [M+H]⁺.

### Example 113: Synthesis of Compound 190

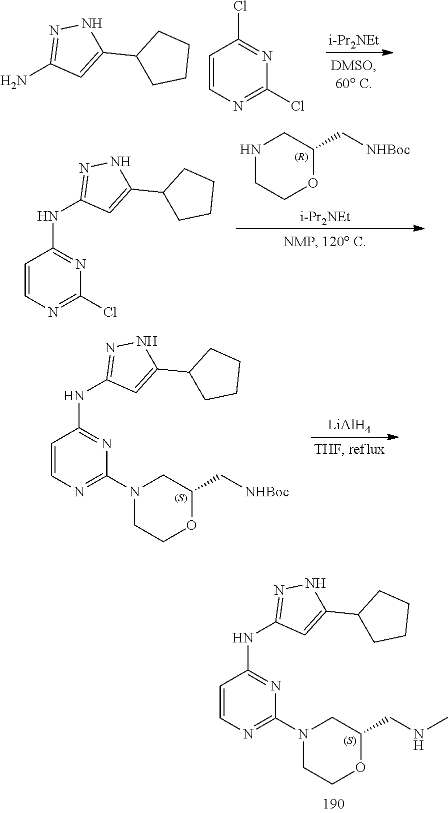

### N-(5-Cyclopentyl-1H-pyrazol-3-yl)-2-[(2S)-2-(methylaminomethyl)morpholin-4-yl]pyrimidin-4-amine

To a solution of tert-butyl N-[[(2S)-4-[4-[(5-cyclopentyl-1H-pyrazol-3-yl)amino]pyrimidin-2-yl]morpholin-2-yl]methyl]carbamate (150 mg, 0.338 mmol) in anhydrous THF (10 mL) was added a 2.4 M solution of lithium aluminum hydride in THF (1.4 mL, 3.4 mmol) dropwise at 0° C. The reaction mixture was then heated to reflux and stirred for 5 hours. The reaction mixture was cooled to 0° C., then quenched by the addition of water (150 μL), followed by a 15% w/v aqueous NaOH solution (150 μL), followed by water (450 μL). The reaction mixture was warmed to room temperature, then stirred for 10 minutes. MgSO₄ (ca. 2 g) was then added, and the reaction mixture was stirred for a further 10 minutes. The reaction mixture was passed through a pad of celite, then the filter cake was washed with CH₂Cl₂ (10 mL), followed by EtOH (10 mL). The filtrate was concentrated to dryness under reduced pressure. The residue was purified by reversed-phase basic prepHPLC to afford the title compound as a solid (44 mg, 0.12 mmol, 36%).

¹H NMR (400 MHz, DMSO-d₆) δ 11.92 (s, 1H), 9.43 (s, 1H), 7.88 (d, J=5.7 Hz, 1H), 6.42-6.01 (m, 2H), 4.55-4.45 (m, 1H), 4.38-4.32 (m, 1H), 3.93-3.85 (m, 1H), 3.47 (ddt, J=11.5, 8.6, 2.7 Hz, 2H), 2.99 (p, J=8.0 Hz, 1H), 2.93-2.83 (m, 1H), 2.68-2.57 (m, 2H), 2.53 (d, J=5.7 Hz, 1H), 2.29 (s, 3H), 2.04-1.94 (m, 2H), 1.76-1.50 (m, 6H).

UPLC-MS (basic method, 4 min): rt=1.35 min, m/z=358.4 [M+H]⁺.

### Example 114: Synthesis of Compound 191

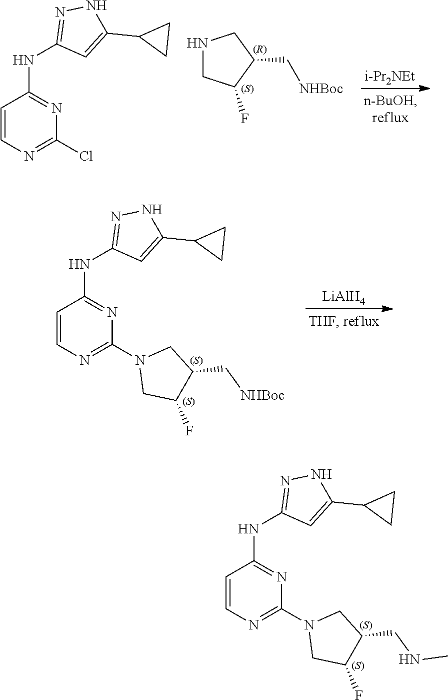

### tert-Butyl N-[[(3S,4S)-1-[4-[(5-cyclopropyl-1H-pyrazol-3-yl)amino]pyrimidin-2-yl]-4-fluoro-pyrrolidin-3-yl]methyl]carbamate

2-chloro-N-(5-cyclopropyl-1H-pyrazol-3-yl)pyrimidin-4-amine (250 mg, 1.06 mmol), tert-butyl N-[[(3R,4S)-4-fluoropyrrolidin-3-yl]methyl]carbamate (250 mg, 1.15 mmol), and N,N-diisopropylethylamine (0.46 mL, 2.7 mmol) were dissolved in n-butanol (5 mL) and the reaction mixture was heated at reflux for 2 h. The reaction mixture was cooled to room temperature and concentrated to dryness under reduced pressure. The residue was diluted with water (50 mL), then extracted with 20% MeOH in DCM (2×50 mL). The combined organic extracts were dried over Na₂SO₄ and the solvent was removed under reduced pressure to afford the title compound as a solid (437 mg, 1.05 mmol, 99%).

¹H NMR δ 11.87 (s, 1H), 9.37 (s, 1H), 7.81 (d, J=5.8 Hz, 1H), 7.00 (s, 1H), 6.20 (s, 1H), 5.23 (d, J=53.7 Hz, 1H), 3.86-3.65 (m, 2H), 3.24-3.09 (m, 2H), 1.82 (td, J=8.6, 4.4 Hz, 1H), 1.36 (s, 9H), 1.31-1.13 (m, 3H), 0.84 (s, 2H), 0.69-0.56 (m, 2H).

UPLC-MS analysis: (2 min, basic): rt=1.00 min, m/z=418.3 [M+H]+.

### N-(5-Cyclopropyl-1H-pyrazol-3-yl)-2-[(3S,4S)-3-fluoro-4-(methylaminomethyl)pyrrolidin-1-yl]pyrimidin-4-amine

To a solution of tert-butyl N-[[(3S,4S)-1-[4-[(5-cyclopropyl-1H-pyrazol-3-yl)amino]pyrimidin-2-yl]-4-fluoro-pyrrolidin-3-yl]methyl]carbamate (130 mg, 0.311 mmol) in anhydrous THF (10 mL) was added 2.4 M lithium aluminium hydride in THF (1.3 mL, 3.1 mmol) dropwise under a nitrogen atmosphere at 0° C. The reaction mixture was then heated to reflux and stirred for 3 hours. The reaction mixture was cooled to 0° C. and then quenched by dropwise addition of water (3 mL), followed by a 15% w/v aqueous NaOH solution (3 mL), followed by water (10 mL). The quenched reaction mixture was warmed to room temperature and stirred for 15 minutes. Ethyl acetate was added (30 mL), and the resulting organic phase was washed with water (20 mL) then a saturated aqueous brine solution (20 mL). The organic phase was dried over Na₂SO₄ and concentrated to dryness under reduced pressure. The residue was purified by reversed-phase prepHPLC to afford the title compound as a solid (9.0 mg, 27 μmol, 9%).

¹H NMR analysis: (400 MHz, DMSO-d₆) δ 11.87 (s, 1H), 9.35 (s, 1H), 7.81 (d, J=5.7 Hz, 1H), 6.18 (s, 1H), 5.40-5.10 (m, 1H), 3.87-3.71 (m, 2H), 3.63 (d, J=41.5 Hz, 1H), 3.09 (s, 1H), 2.73 (s, 1H), 2.65-2.48 (m, 2H), 2.29 (s, 3H), 1.82 (tt, J=8.6, 4.9 Hz, 1H), 0.94-0.81 (m, 2H), 0.67-0.56 (m, 2H).

¹⁹F NMR (376 MHz, DMSO-d₆) δ −192.4-−192.8 ppm.

UPLC-MS analysis: (4 min, basic): rt=1.16 min, m/z=332.3 [M+H]⁺.

### Example 115: Synthesis of Compound 155

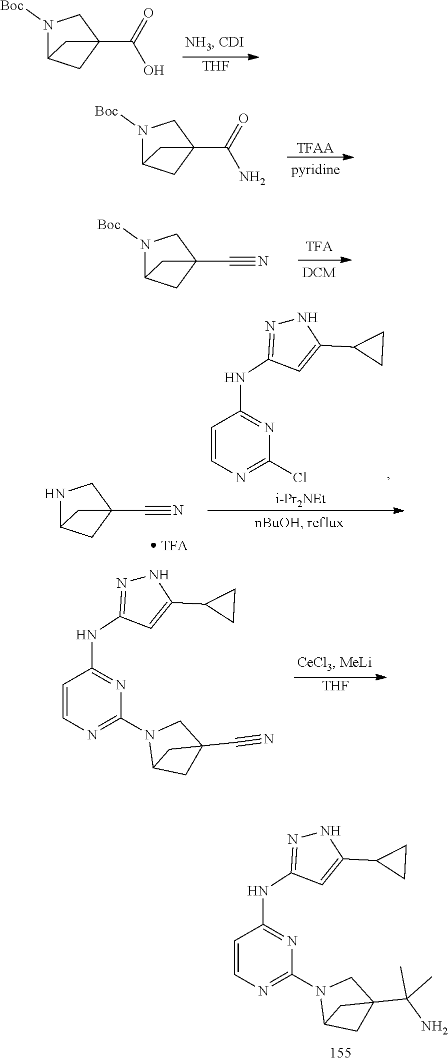

### 1-[2-[4-[(5-cyclopropyl-1H-pyrazol-3-yl)amino]pyrimidin-2-yl]-2-azabicyclo[2.1.1]hexan-4-yl]ethenone

2-[4-[(5-cyclopropyl-1H-pyrazol-3-yl)amino]pyrimidin-2-yl]-2-azabicyclo[2.1.1]hexane-4-carbonitrile (500 mg, 1.63 mmol) was dissolved in THE (15 mL) at 0° C. and anhydrous cerium(III)chloride (1.20 g, 4.88 mmol) was added to it. The reaction mixture was stirred for 20 min. 1.6 M methyllithium in diethyl ether (20 mL, 32.5 mmol) was added to it and reaction mixture was stirred for 4.5 hours. The reaction mixture was quenched by adding a saturated aqueous ammonium chloride solution (10 mL) and extracted with 1:4 v/v MeOH:DCM solution (2×20 mL). The combined organic extracts were separated and dried over sodium sulfate, then the solvent was evaporated. The crude material was purified by reversed-phase basic prepHPLC to afford to afford the title compound as a solid (20 mg, 62 μmol, 4%).

¹H NMR (400 MHz, DMSO-d₆) δ 11.92 (s, 1H), 9.35 (s, 1H), 7.83 (d, J=5.7 Hz, 1H), 6.24 (s, 1H), 6.14 (s, 1H), 4.63 (t, J=1.8 Hz, 1H), 3.35 (s, 2H), 1.91-1.83 (m, 1H), 1.83-1.76 (m, 2H), 1.41 (s, 2H), 1.30 (dd, J=4.3, 1.8 Hz, 2H), 1.05 (s, 6H), 0.96-0.85 (m, 2H), 0.69-0.61 (m, 2H).

UPLC-MS (4 min, basic method): rt=1.23 min, m/z=340.2 [M+H]⁺.

### Example 116: Synthesis of Compound 164

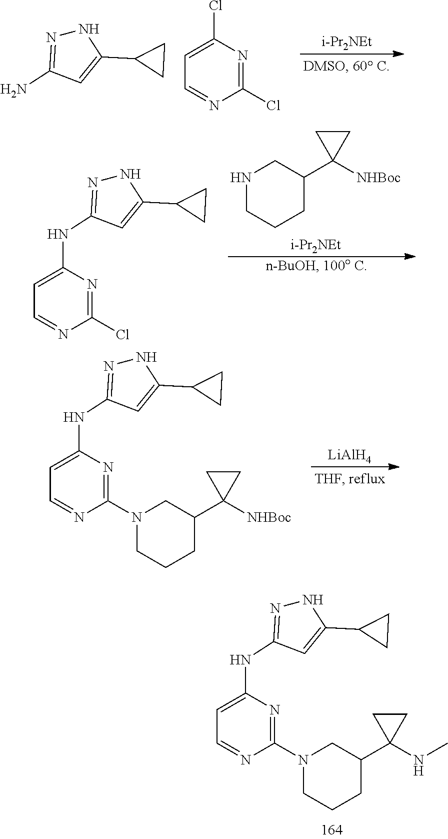

### tert-Butyl N-[1-[1-[4-[(5-Cyclopropyl-1H-pyrazol-3-yl)amino]pyrimidin-2-yl]-3-piperidyl]-1-methyl-propyl]carbamate

To a solution of 2-chloro-N-(5-cyclopropyl-1H-pyrazol-3-yl)pyrimidin-4-amine (404 mg, 1.71 mmol) and tert-butyl N-(1-piperidin-3-ylcyclopropyl)carbamate (474 mg, 1.97 mmol) in n-butanol (17 mL) was added N,N-diisopropylethylamine (0.90 mL, 5.1 mmol). The resulting solution was then heated to 100° C. and stirred for 16 h. The reaction mixture was concentrated under reduced pressure and diluted with water (40 mL). The compound was extracted with DCM (3×20 mL). The combined organic fractions were dried over Na₂SO₄ and concentrated to dryness to afford the title compound as a solid (0.77 g, 1.6 mmol, 93%).

¹H NMR (400 MHz, DMSO-d₆) δ 11.90 (s, 1H), 9.31 (s, 1H), 7.82 (d, J=5.7 Hz, 1H), 7.12 (s, 1H), 6.17 (s, 2H), 4.68 (dd, J=50.4, 12.9 Hz, 2H), 4.31 (t, J=5.2 Hz, 1H), 3.42-3.33 (m, 2H), 2.66-2.53 (m, 1H), 1.93-1.60 (m, 3H), 1.43-1.37 (m, 3H), 1.37 (s, 9H), 1.34-1.24 (m, 3H), 1.04 (d, J=6.1 Hz, 1H), 0.95-0.90 (m, 1H), 0.61 (d, J=5.4 Hz, 1H).

UPLC-MS (basic method, 4 min): rt=1.11 min, m z: 440.5 [M+H]⁺.

### N-(5-Cyclopropyl-1H-pyrazol-3-yl)-2-[3-[1-(methylamino)cyclopropyl]-1-piperidyl]pyrimidin-4-amine

To a solution of tert-butyl N-[1-[1-[4-[(5-cyclopropyl-1H-pyrazol-3-yl)amino]pyrimidin-2-yl]-3-piperidyl]cyclopropyl]carbamate (610 mg, 1.39 mmol) in anhydrous THE (14 mL) under an inert atmosphere was added 2.4 M solution of lithium aluminum hydride in THE (5.8 mL, 13.9 mmol) at 0° C. The resulting mixture was allowed to warm to room temperature then heated to reflux and stirred for 4 hours. The reaction mixture was allowed to cool to room temperature and water (10 mL) was added dropwise, followed by 10% w/v aqueous NaOH solution (10 mL) and then additional water (10 mL). Ethyl acetate (25 mL) was then added, and the resulting organic phase was washed sequentially with water (2×20 mL) and saturated brine solution (20 mL). The organic fraction was dried (Na₂SO₄) and concentrated to under reduced pressure. The residue was purified by reversed-phase basic prepHPLC. The residue was then dissolved in MeOH loaded onto an SCX-2 cartridge. The cartridge was then washed with methanol (40 mL) and the compound was eluted with 2 M NH₃ in methanol (20 mL). The volatiles were removed under reduced pressure to afford the title compound as a solid (56 mg, 0.16 mmol, 11%).

¹H NMR (400 MHz, DMSO-d₆) δ 11.92 (s, 1H), 9.34 (s, 1H), 7.83 (d, J=5.7 Hz, 1H), 6.17 (s, 2H), 4.68 (dd, J=29.6, 12.8 Hz, 2H), 2.70-2.53 (m, 2H), 2.23 (s, 3H), 1.93-1.80 (m, 1H), 1.78-1.61 (m, 2H), 1.37-1.25 (m, 3H), 0.97-0.88 (m, 2H), 0.88-0.82 (m, 1H), 0.71-0.63 (m, 2H), 0.50-0.38 (m, 4H).

UPLC-MS (basic method, 4 min): rt=1.50 min, m z: 354.3 [M+H]⁺.

### Example 117: Synthesis of Compound 176

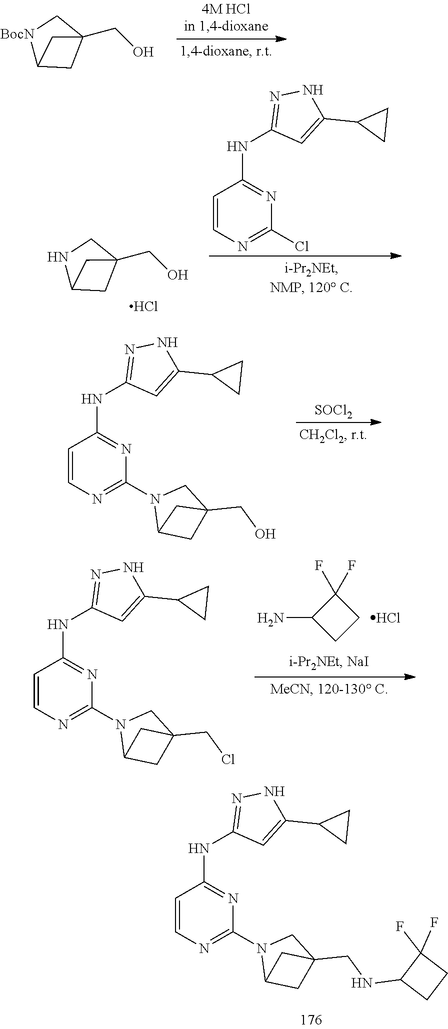

### N-(5-Cyclopropyl-1H-pyrazol-3-yl)-2-[4-[[(2,2-difluorocyclobutyl)amino]methyl]-2-azabicyclo[2.1.1]hexan-2-yl]pyrimidin-4-amine

A suspension of 2-[4-(chloromethyl)-2-azabicyclo[2.1.1]hexan-2-yl]-N-(5-cyclopropyl-1H-pyrazol-3-yl)pyrimidin-4-amine (150 mg, 0.453 mmol), 2,2-difluorocyclobutan-1-amine hydrochloride (325 mg, 2.27 mmol), N,N-diisopropylethylamine (0.63 mL, 3.6 mmol) and sodium iodide (68 mg, 0.45 mmol) in MeCN (3 mL) was heated to 120° C. in a sealed tube and stirred for 24 hours. The reaction mixture was heated to 130° C. and stirred for a further 9 days. The reaction mixture was then diluted with 0.5 M aqueous NaOH solution (10 mL), then the compound was extracted with 3:1 CHCl₃:IPA (3×10 mL). The combined organic extracts were dried over Na₂SO₄, then concentrated to dryness under reduced pressure. The residue was purified by reversed-phase basic prepHPLC to afford the title compound as a solid (23 mg, 57 μmol, 13%).

¹H NMR (400 MHz, Chloroform-d) δ 8.00 (d, J=5.7 Hz, 1H), 6.83 (s, 1H), 6.08 (s, 1H), 5.84 (s, 1H), 4.85 (s, 1H), 3.64-3.41 (m, 3H), 3.12 (d, J=12.3 Hz, 1H), 2.91 (d, J=12.4 Hz, 1H), 2.38-2.06 (m, 3H), 1.92-1.81 (m, 3H), 1.62-1.52 (m, 2H), 1.50-1.36 (m, 1H), 1.01-0.88 (m, 2H), 0.78-0.70 (m, 2H).

¹⁹F NMR (376 MHz, Chloroform-d) δ −83.7-−84.9 (m), −122.6-−123.5 (m).

UPLC-MS (basic method, 4 min): Rt: 1.51 min, m/z=402.4 [M+H]⁺.

### Example 118: Synthesis of Compound 180

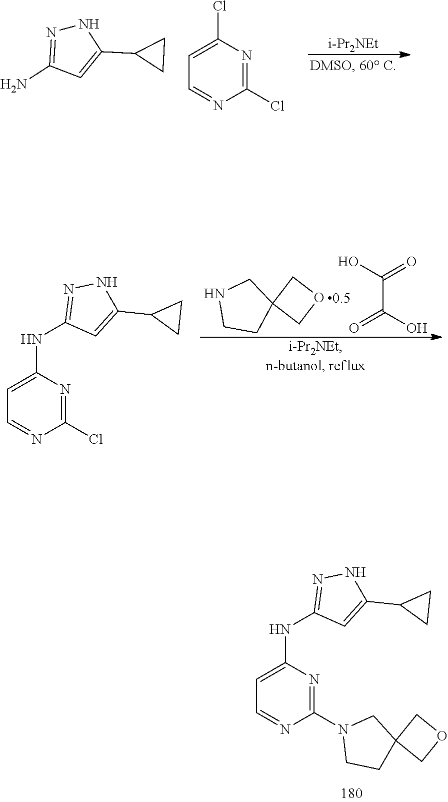

### N-(5-Cyclopropyl-1H-pyrazol-3-yl)-2-(2-oxa-7-azaspiro[3.4]octan-7-yl)pyrimidin-4-amine

A stirred mixture of 2-chloro-N-(5-cyclopropyl-1H-pyrazol-3-yl)pyrimidin-4-amine (100 mg, 0.424 mmol), 2-oxa-6-azaspiro[3.4]octane hemioxalate (81 mg, 0.255 mmol) and N,N-diisopropylethylamine (0.37 mL, 2.1 mmol) in n-butanol (4.2 mL) was heated, at reflux, for 18 hours. The reaction mixture was cooled to room temperature and then solvent was removed under reduced pressure to leave a viscous oil. The oil was diluted with water (10 mL) and extracted with DCM (3×10 mL). The combined organic extracts were washed with brine (10 mL) and dried over Na₂SO₄. The solvent was removed under reduced pressure to leave a gum. The crude material was purified by column chromatography over silica (4 g cartridge), loaded in DCM (2.0 mL) and eluting with a gradient of methanol (0% to 10%; v/v) in DCM to afford the title compound as a solid (41 mg, 0.12 mmol, 29%).

¹H NMR (400 MHz, DMSO-d₆) δ 11.89 (s, 1H), 9.39 (s, 1H), 7.83 (d, J=5.7 Hz, 1H), 6.27-6.19 (m, 2H), 4.58 (d, J=6.0 Hz, 2H), 4.51 (d, J=6.0 Hz, 2H), 3.69 (s, 2H), 3.47 (t, J=6.9 Hz, 2H), 2.21 (t, J=6.9 Hz, 2H), 1.91-1.82 (m, 1H), 0.94-0.88 (m, 2H), 0.69-0.64 (m, 2H).

UPLC-MS (basic method, 4 min): Rt: 1.20 min, m/z=313.2 [M+H]⁺.

### Example 119: Synthesis of Compound 184

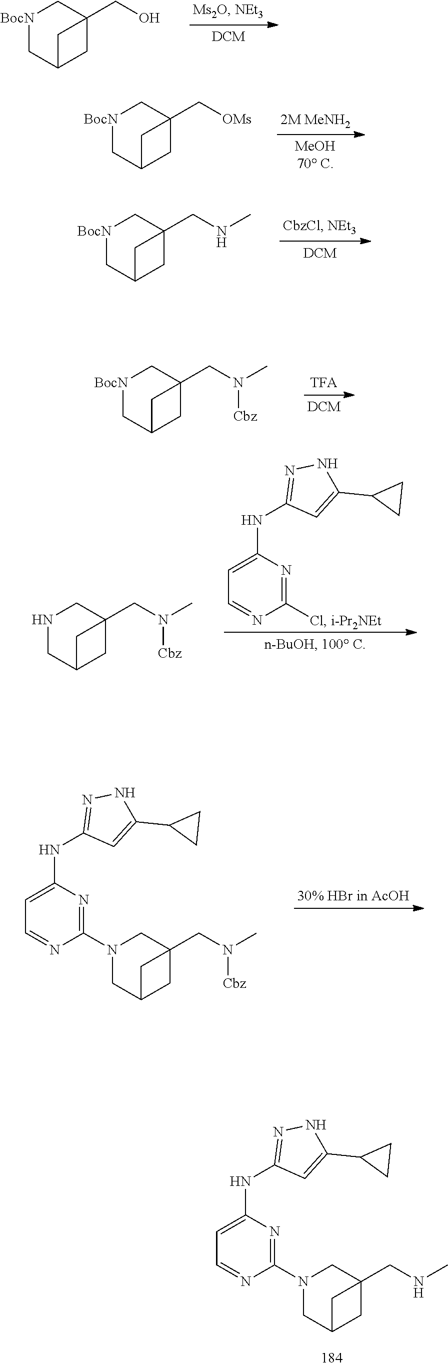

### tert-Butyl 1-(methylsulfonyloxymethyl)-3-azabicyclo[3.1.1]heptane-3-carboxylate

A solution of tert-butyl 1-(hydroxymethyl)-3-azabicyclo[3.1.1]heptane-3-carboxylate (0.98 g, 4.32 mmol) in DCM (45 mL) was cooled to 0° C. Triethylamine (1.8 mL, 12.9 mmol) was added, followed by methanesulfonic anhydride (1.50 g, 8.63 mmol). The reaction was allowed to warm to ambient temperature and stirred for 16 h. Water (50 mL) was added to the reaction, the layers partitioned, and the aqueous layer extracted with DCM (2×50 mL). The combined organics were washed with a saturated brine solution (50 mL), dried over Na₂SO₄, filtered and concentrated to dryness to afford the title compound as a solid (1.31 g, 4.13 mmol, 96%).

¹H NMR (400 MHz, DMSO-d₆) δ 4.11 (s, 2H), 3.45-3.33 (m, 4H), 3.18 (s, 3H), 2.41 (dtd, J=15.5, 6.3, 3.2 Hz, 1H), 1.95 (t, J=7.0 Hz, 2H), 1.42 (d, J=1.4 Hz, 9H), 1.38-1.30 (m, 2H).

UPLC-MS-ELS (basic method, 4 min): rt=1.71 min, m z: 250.1 [M−tBu+H]⁺.

### tert-Butyl 1-(methylaminomethyl)-3-azabicyclo[3.1.1]heptane-3-carboxylate

A mixture of tert-butyl 1-(methylsulfonyloxymethyl)-3-azabicyclo[3.1.1]heptane-3-carboxylate (1.31 g, 4.29 mmol), a 40% w/v solution of methylamine in water (3.6 mL, 43 mmol) and methanol (22 mL) was heated to 70° C. in a sealed tube, and stirred for 16 h. The reaction mixture was allowed to cool to ambient temperature, concentrated to dryness and the residue was suspended in an aqueous 0.5 M NaOH solution (25 mL) then extracted with EtOAc (3×20 mL). The combined organic extracts were washed with a saturated brine solution (20 mL) and dried over Na₂SO₄, then concentrated to dryness under reduced pressure to afford the title compound as an oil (0.86 g, 3.59 mmol, 84%).

¹H NMR (400 MHz, Chloroform-d) δ 3.48 (dd, J=9.6, 2.6 Hz, 2H), 3.40 (s, 2H), 2.50 (d, J=3.9 Hz, 2H), 2.48-2.38 (m, 4H), 2.39-2.32 (m, 1H), 1.93-1.79 (m, 2H), 1.51-1.42 (m, 9H), 1.37 (td, J=7.2, 2.8 Hz, 2H).

UPLC-MS (basic method, 4 min, ELSD): rt=1.50 min, m z: 185.1 [M−tBu+H]⁺.

### tert-butyl 1-[[benzyloxycarbonyl(methyl)amino]methyl]-3-azabicyclo[3.1.1]heptane-3-carboxylate

To a solution of tert-butyl 1-(methylaminomethyl)-3-azabicyclo[3.1.1]heptane-3-carboxylate (863 mg, 3.59 mmol) and triethylamine (0.75 mL, 5.4 mmol) in DCM (10 mL) was added benzyl chloroformate (0.56 mL, 3.95 mmol) dropwise at 0° C. The reaction mixture was then allowed to warm to ambient temperature and stirred for 16 h. The reaction mixture was diluted with water (50 mL) and the organics were extracted with DCM (3×40 mL). The combined organic extracts were then dried over Na₂SO₄, filtered and concentrated to afford the title compound as an oil (1.27 g, 3.22 mmol, 90%).

¹H NMR (400 MHz, Chloroform-d) δ 7.43-7.28 (m, 5H), 5.13-5.08 (m, 2H), 3.52-3.33 (m, 4H), 3.31-3.19 (m, 2H), 2.98-2.90 (m, 3H), 1.95-1.84 (m, 2H), 1.68-1.63 (m, 1H), 1.50-1.45 (m, 9H), 1.45-1.32 (m, 2H).

UPLC-MS (basic method, 4 min, ELSD): rt=2.20 min, m z: 375.3 [M+H]⁺.

### Benzyl N-(3-Azabicyclo[3.1.1]heptan-1-ylmethyl)-N-methyl-carbamate

To a solution of tert-butyl 1-[[benzyloxycarbonyl(methyl)amino]methyl]-3-azabicyclo[3.1.1]heptane-3-carboxylate (1.25 g, 3.34 mmol) in DCM (20 mL) was added trifluoroacetic acid (5.1 mL, 67 mmol) dropwise at 0° C. The reaction mixture was allowed to warm to ambient temperature and stirred for 3 hours. The reaction mixture was then concentrated to an oil under reduced pressure, then was diluted with DCM (20 mL), and the resulting solution washed with water (3×100 mL) and saturated brine solution (100 mL). The organic fraction was dried over Na₂SO₄ and concentrated to dryness to afford the title compound as an oil (610 mg, 1.96 mmol, 59%).

¹H NMR (400 MHz, Chloroform-d) δ 7.39-7.28 (m, 5H), 5.30 (s, 2H), 5.13-5.08 (m, 2H), 3.21 (m, 4H), 3.08-3.01 (m, 1H), 2.97-2.89 (m, 3H), 2.43-2.29 (m, 1H), 2.05-1.89 (m, 2H), 1.73-1.53 (m, 2H).

UPLC-MS-ELS (basic method, 2 min): rt=0.87 min, m z: 275.2 [M+H]⁺.

### Benzyl N-[[3-[4-[(5-Cyclopropyl-1H-pyrazol-3-yl)amino]pyrimidin-2-yl]-3-azabicyclo[3.1.1]heptan-1-yl]methyl]-N-methyl-carbamate

To a solution of 2-chloro-N-(5-cyclopropyl-1H-pyrazol-3-yl)pyrimidin-4-amine (382 mg, 1.62 mmol) and benzyl N-(3-azabicyclo[3.1.1]heptan-1-ylmethyl)-N-methyl-carbamate (581 mg, 1.86 mmol) in n-butanol (15 mL) was added N,N-diisopropylethylamine (0.85 mL, 4.9 mmol). The reaction mixture was heated to 100° C. and allowed to stir for 16 h. The reaction mixture was concentrated under reduced pressure and diluted with water (30 mL). The mixture was then extracted with DCM (3×20 mL). The combined organic fractions were washed with saturated brine solution (20 mL), dried over Na₂SO₄ and concentrated to dryness to afford an oil. The crude material was purified by flash column chromatography over silica gel, eluting with a gradient of 2 M NH₃ in MeOH (0% to 10% v/v) in DCM to afford the title compound as a solid (405 mg, 0.573 mmol, 35%).

¹H NMR (400 MHz, DMSO-d₆) δ 11.88 (s, 1H), 9.39 (s, 1H), 7.88 (s, 1H), 7.46-7.26 (m, 5H), 6.26 (s, 2H), 5.12-5.05 (m, 2H), 3.72-3.48 (m, 4H), 3.01-2.86 (m, 3H), 1.98-1.80 (m, 3H), 1.40 (s, 3H), 0.96-0.82 (m, 3H), 0.71-0.61 (m, 3H).

UPLC-MS (basic method, 4 min): rt=1.91 min, m z: 474.3 [M+H]⁺.

### N-(5-Cyclopropyl-1H-pyrazol-3-yl)-2-[1-(methylaminomethyl)-3-azabicyclo[3.1.1]heptan-3-yl]pyrimidin-4-amine

To benzyl N-[[3-[4-[(5-cyclopropyl-1H-pyrazol-3-yl)amino]pyrimidin-2-yl]-3-azabicyclo[3.1.1]heptan-1-yl]methyl]-N-methyl-carbamate (386 mg, 0.546 mmol) was added 30% HBr in acetic acid (5 mL), and the reaction was stirred for 3 hours. The reaction mixture was then concentrated under reduced pressure, and the residue loaded onto an SCX-2 cartridge. The cartridge was washed with MeOH (50 mL) and the compound was eluted with 2 M NH₃ in MeOH (30 mL). The eluent was concentrated to dryness under reduced pressure to afford a yellow oil. The residue was purified by reversed-phase basic prepHPLC. The residue was dissolved in MeOH (1 mL), loaded onto an SCX-2 cartridge, washed with MeOH (30 mL) and eluted with 2 M NH₃ in MeOH (20 mL). The eluent was concentrated to dryness under reduced pressure to afford the title compound as a powder (26 mg, 78 μmol, 14%).

¹H NMR (400 MHz, Methanol-d₄) δ 7.89 (d, J=5.9 Hz, 1H), 6.18 (s, 1H), 3.68 (s, 4H), 2.65 (s, 2H), 2.58 (s, 1H), 2.46 (s, 3H), 2.10-2.04 (m, 2H), 1.89 (tt, J=9.1, 5.0 Hz, 1H), 1.49 (dd, J=6.8, 2.8 Hz, 2H), 0.97 (d, J=8.2 Hz, 2H), 0.75-0.69 (m, 2H).

¹H NMR (400 MHz, DMSO-d₆) δ 11.94 (s, 1H), 9.39 (s, 1H), 7.86 (d, J=5.7 Hz, 1H), 6.25 (s, 2H), 3.71-3.51 (m, 4H), 2.32 (d, J=10.9 Hz, 3H), 1.90 (s, 2H), 1.83 (s, 1H), 1.35 (dd, J=6.8, 2.5 Hz, 2H), 0.91 (d, J=8.1 Hz, 2H), 0.65 (s, 2H).

UPLC-MS (basic method, 4 min): rt=1.33 min, m z: 340.3 [M+H]⁺.

### Example 120: Synthesis of Compound 185

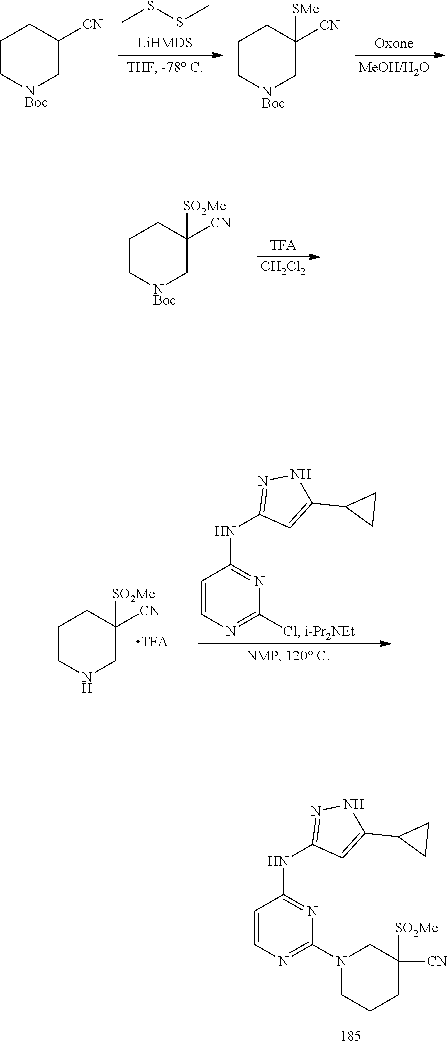

### tert-Butyl 3-Cyano-3-methylsulfanyl-piperidine-1-carboxylate

To a solution of 1-Boc-piperidine-3-carbonitrile (5.00 g, 23.8 mmol) in THF (10 mL) under nitrogen atmosphere was added a 1.0 M solution of lithium bis(trimethylsilyl)amide in THF (26 mL, 26 mmol) at −78° C. dropwise. After 30 minutes of stirring, a solution of dimethyl disulfide (2.1 mL, 24 mmol) in THF (1 mL) was added to it. The reaction mixture was stirred for 2 hours at −78° C. Afterwards, the reaction mixture was quenched with a saturated aqueous solution of ammonium chloride (10 mL) then extracted with EtOAc (50 mL). The organic layer was separated, washed with brine (50 mL), and dried under reduced pressure to afford the title compound as an oil (5.80 g, 22.6 mmol, 95%).

¹H NMR (400 MHz, Chloroform-d) δ 4.34-4.00 (m, 1H), 3.84 (d, J=26.5 Hz, 1H), 3.24-2.88 (m, 2H), 2.30 (s, 3H), 2.26-2.18 (m, 1H), 1.87-1.58 (m, 3H), 1.46 (s, 9H).

UPLC-MS-ELS (2 minute, basic method) rt=1.15 min, m/z=257.1 [M+H]⁺.

### tert-Butyl 3-Cyano-3-methylsulfonyl-piperidine-1-carboxylate

To a solution of tert-butyl 3-cyano-3-methylsulfanyl-piperidine-1-carboxylate (1.12 g, 4.02 mmol) in methanol (8 mL) and water (2 mL) was added Oxone® (2.45 g, 16.1 mmol) portion-wise at 0° C. The reaction mixture was stirred for 72 hours at the room temperature, then was quenched with water (10 mL). The compound was extracted with dichloromethane (2×20 mL), dried over sodium sulfate, and concentrated under reduced pressure to afford the title compound as an oil (675 mg, 2.27 mmol, 56%).

¹H NMR (400 MHz, Chloroform-d) 4.84-4.54 (m, 1H), 4.34-3.92 (m, 1H), 3.20 (d, J=13.4 Hz, 1H), 3.12 (s, 3H), 2.86-2.55 (m, 1H), 2.42-2.31 (m, 1H), 2.17-2.05 (m, 1H), 1.97-1.77 (m, 2H), 1.46 (s, 9H).

UPLC-MS-ELS (2 minute, basic method) rt=0.75 min, m/z=289.1 [M+H]⁺.

### 3-Methylsulfonylpiperidine-3-carbonitrile;2,2,2-trifluoroacetic acid

To a solution of tert-butyl 3-cyano-3-methylsulfonyl-piperidine-1-carboxylate (590 mg, 2.05 mmol) in DCM (6 mL) was added trifluoroacetic acid (6.0 mL, 2.1 mmol) dropwise at 0° C. The reaction mixture was stirred for 1.5 hour at the same temperature. The volatiles were removed under reduced pressure and methanol was added to it. After stirring for 10 minutes, the precipitate was collected by filtration to afford the title compound as a solid (590 mg, 1.95 mmol, 95%).

¹H NMR (400 MHz, DMSO-d₆) δ 4.09-4.01 (m, 1H), 3.40 (s, 3H), 3.36 (s, 1H), 3.32-3.17 (m, 1H), 3.03-2.92 (m, 1H), 2.43-2.34 (m, 1H), 2.19-2.01 (m, 2H), 1.84-1.68 (m, 1H). 1-[4-[(5-Cyclopropyl-1H-pyrazol-3-yl)amino]pyrimidin-2-yl]-3-methylsulfonyl-piperidine-3-carbonitrile

To a stirred solution of 2-chloro-N-(5-cyclopropyl-1H-pyrazol-3-yl)pyrimidin-4-amine (300 mg, 1.27 mmol) in NMP (3 mL) was added 3-methylsulfonylpiperidine-3-carbonitrile;2,2,2-trifluoroacetic acid (577 mg, 1.91 mmol) and N,N-diisopropylethylamine (0.89 mL, 5.1 mmol). The reaction mixture was heated to 120° C. and stirred for 96 hours. The reaction mixture was cooled to room temperature, diluted with cold water (ca. 100 mL), and stirred for 30 minutes. The precipitate was collected by filtration. The solid was then triturated in diethylether:hexane. The solid was then purified by reversed-phase preparative HPLC to afford the title compound as a solid (28 mg, 72 μmol, 6%).

¹H NMR (400 MHz, Chloroform-d) δ 7.95 (dd, J=5.8, 0.9 Hz, 1H), 6.20 (d, J=5.8 Hz, 1H), 6.00 (s, 1H), 5.51 (d, J=13.3 Hz, 1H), 4.82 (d, J=13.6 Hz, 1H), 3.29 (d, J=13.2 Hz, 1H), 3.19 (s, 3H), 2.87-2.76 (m, 1H), 2.44 (d, J=13.1 Hz, 1H), 2.28-2.16 (m, 2H), 2.04-1.85 (m, 2H), 1.87-1.76 (m, 2H), 0.80-0.66 (m, 2H).

UPLC MS (4 minute, basic method), rt=1.35 min, m/z=388.2 [M+H]⁺.

### Example 121: Synthesis of Compound 186

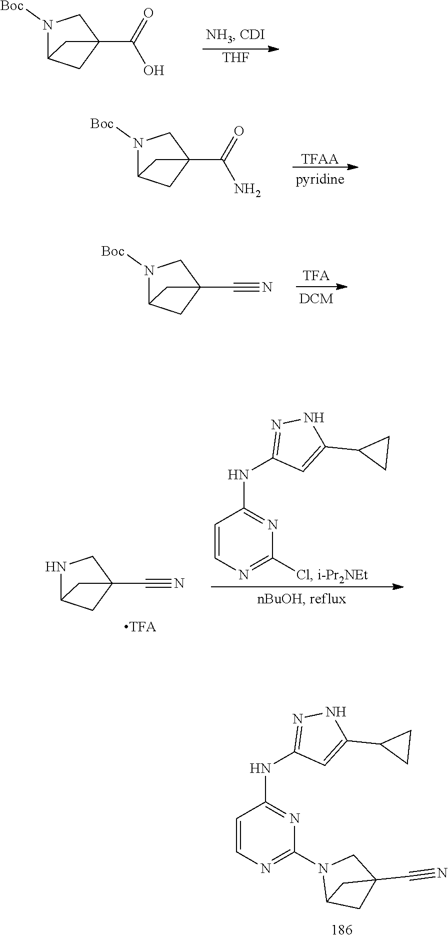

### tert-Butyl 4-Carbamoyl-2-azabicyclo[2.1.1]hexane-2-carboxylate

2-tert-butoxycarbonyl-2-azabicyclo[2.1.1]hexane-4-carboxylic acid (4.32 g, 19.0 mmol) and N,N-carbonyldiimidazole (4.62 g, 28.5 mmol) were dissolved in THE (50 mL) and the reaction mixture was stirred at room temperature for 4 hours. Then, a 0.5 M solution of ammonia in THE (76 mL, 38.0 mmol) was added to it and reaction mixture was stirred at room temperature for a further 24 hours. The solvent was evaporated, and ice water was added to the crude material, resulting in a precipitation. The precipitated solid was collected by filtration, and the filter cake was washed with water (50 mL), then dried under suction to afford the title compound as a solid (2.70 g, 11.9 mmol, 63%).

¹H NMR (400 MHz, DMSO-d₆) δ 7.36 (s, 1H), 7.11 (s, 1H), 4.09 (t, J=1.8 Hz, 1H), 3.27 (s, 2H), 2.06-1.96 (m, 2H), 1.54-1.44 (m, 2H), 1.36 (s, 9H).

UPLC-MS analysis: (2 min, basic): rt=0.84 min, m/z=225.2 [M−H]⁺.

### tert-Butyl 4-Cyano-2-azabicyclo[2.1.1]hexane-2-carboxylate

tert-butyl 4-carbamoyl-2-azabicyclo[2.1.1]hexane-2-carboxylate (2.40 g, 10.6 mmol) was dissolved in pyridine (2.6 mL) at 0° C. and trifluoroacetic anhydride (2.2 mL, 16 mmol) was added slowly to the reaction mixture. Reaction mixture was warmed to room temperature and stirred for 24 h. The reaction mixture was quenched by adding a 1 M aqueous solution of HCl (20 mL) then extracted with DCM (2×20 mL). The combined organic extracts were separated, then washed with water, brine and dried over sodium sulfate. The solvent was evaporated and crude material was purified by column chromatography, over silica gel, to afford the title compound as a solid (1.92 g, 9.22 mmol, 87%).

¹H NMR (400 MHz, Chloroform-d) δ 4.34 (s, 1H), 3.56 (s, 2H), 2.31 (dt, J=4.7, 1.9 Hz, 2H), 1.84 (dd, J=4.7, 1.9 Hz, 2H), 1.46 (s, 9H).

### 2-Azabicyclo[2.1.1]hexane-4-carbonitrile;2,2,2-trifluoroacetic acid

tert-butyl 4-cyano-2-azabicyclo[2.1.1]hexane-2-carboxylate (1.90 g, 9.12 mmol) was dissolved in DCM (10 mL) and trifluoroacetic acid (3.5 mL, 46 mmol) was added to it at 0° C. and the reaction mixture was stirred for 5 min. The reaction mixture was brought to room temperature and stirred for a further 4 hours. The Reaction mixture was then concentrated to dryness under reduced pressure to afford the title compound as an oil (2.02 g, 9.09 mmol, 99%).

¹H NMR (400 MHz, DMSO-d₆) δ 9.33 (s, 2H), 4.15 (t, J=1.9 Hz, 1H), 3.53 (t, J=3.9 Hz, 2H), 2.52 (dtd, J=5.5, 2.1, 1.1 Hz, 2H), 1.85 (dd, J=5.6, 2.2 Hz, 2H).

### 2-[4-[(5-cyclopropyl-1H-pyrazol-3-yl)amino]pyrimidin-2-yl]-2-azabicyclo[2.1.1]hexane-4-carbonitrile

2-chloro-N-(5-cyclopropyl-1H-pyrazol-3-yl)pyrimidin-4-amine (145 mg, 0.615 mmol), 2-azabicyclo[2.1.1]hexane-4-carbonitrile;2,2,2-trifluoroacetic acid (137 mg, 0.615 mmol), and N,N-diisopropylethylamine (1.1 mL, 6.2 mmol) were dissolved in n-butanol (10 mL) and the reaction mixture was heated to reflux and stirred for 24 h. The solvent was evaporated under reduced pressure and the residue was purified by column chromatography, over silica gel, eluting with 4:1 v/v DCM:MeOH to afford the title compound as a solid (55 mg, 0.18 mmol, 29%).

¹H NMR (400 MHz, DMSO-d₆) δ 11.94 (s, 1H), 9.48 (s, 1H), 7.88 (d, J=5.7 Hz, 1H), 6.35 (s, 1H), 6.13 (s, 1H), 4.68 (t, J=1.9 Hz, 1H), 3.74 (s, 2H), 2.47-2.39 (m, 2H), 1.88 (ddd, J=13.5, 8.5, 5.0 Hz, 1H), 1.83 (dd, J=4.4, 1.8 Hz, 2H), 0.96-0.87 (m, 2H), 0.72-0.63 (m, 2H).

UPLC-MS analysis: (4 min, basic): rt=0.95 min, m/z=308.1 [M+H]⁺.

### Example 122: Synthesis of Compounds 192-1 and 192-2

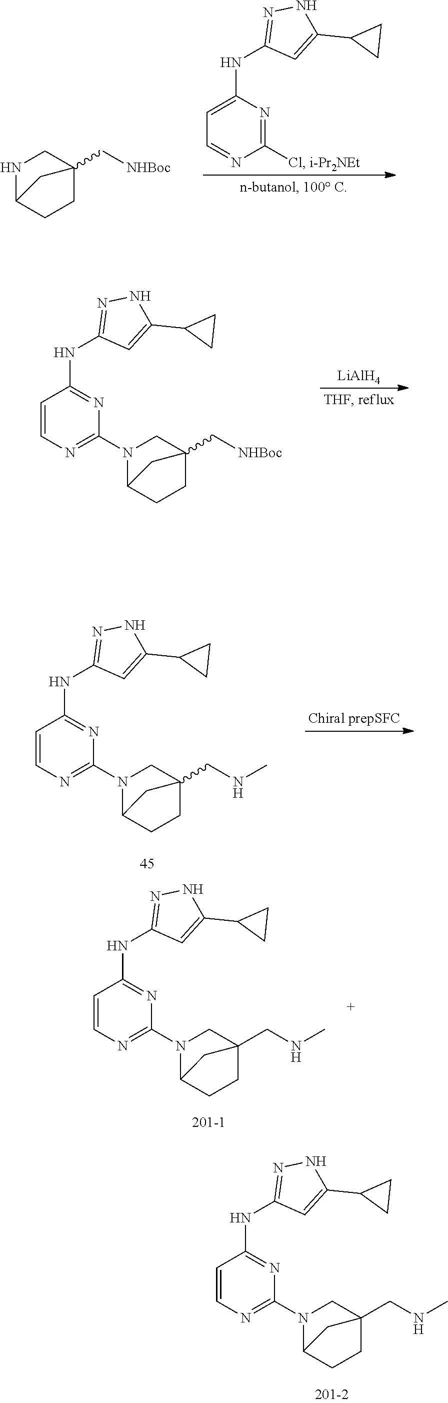

Enantiomer separation by chiral prepSFC was carried out by Reach Separations using a Lux A1 (21.2 mm×250 mm, 5 μm) column and the following parameters: temperature: 40° C.; flow rate: 50 mL/min; mobile phase: 30:70 EtOH:CO₂ (0.2% v/v NH₃), to afford Compound 192-1 as a solid (6.4 mg, 19 μmol) and Compound 192-2 as a solid (5.6 mg, 16 μmol).

### Compound 192-1

¹H NMR (400 MHz, DMSO-d₆) δ 11.89 (s, 1H), 9.28 (s, 1H), 7.79 (d, J=5.7 Hz, 1H), 6.18 (s, 2H), 4.48 (s, 1H), 3.16-3.12 (m, 1H), 2.74-2.62 (m, 2H), 2.38-2.29 (m, 3H), 1.89-1.47 (m, 6H), 1.39 (s, 2H), 0.94-0.88 (m, 2H), 0.67-0.61 (m, 2H).

UPLC-MS (4 min, basic): rt=1.23 min, m/z=340.2 [M+H]⁺.

Chiral HPLC (16 min): Amy-C Neo (4.6 mm×250 mm, 5 m) column; mobile phase: 30:70 MeOH:CO₂ (0.1% v/v NH₃), flow rate: 3 mL/min; temperature: 40° C.; rt=5.81 min (major), 7.92 min (minor), >95% ee.

### Compound 192-2

¹H NMR (400 MHz, DMSO-d₆) δ 11.90 (s, 1H), 9.29 (s, 1H), 7.80 (d, J=5.8 Hz, 1H), 6.18 (s, 2H), 4.50-4.46 (m, 1H), 3.20-3.07 (m, 1H), 2.80-2.68 (m, 2H), 2.37-2.31 (m, 3H), 1.90-1.48 (m, 6H), 1.42-1.37 (m, 2H), 0.98-0.85 (m, 2H), 0.68-0.61 (m, 2H).

UPLC-MS (4 min, basic): rt=1.33 min, m/z=340.2 [M+H]⁺.

Chiral HPLC (16 min): Amy-C Neo (4.6 mm×250 mm, 5 m) column; mobile phase: 30:70 MeOH:CO₂ (0.1% v/v NH₃), flow rate: 3 mL/min; temperature: 40° C.; rt=6.01 min (minor), 7.71 min (major), >96% ee.

### Example 123: Synthesis of Compound 199

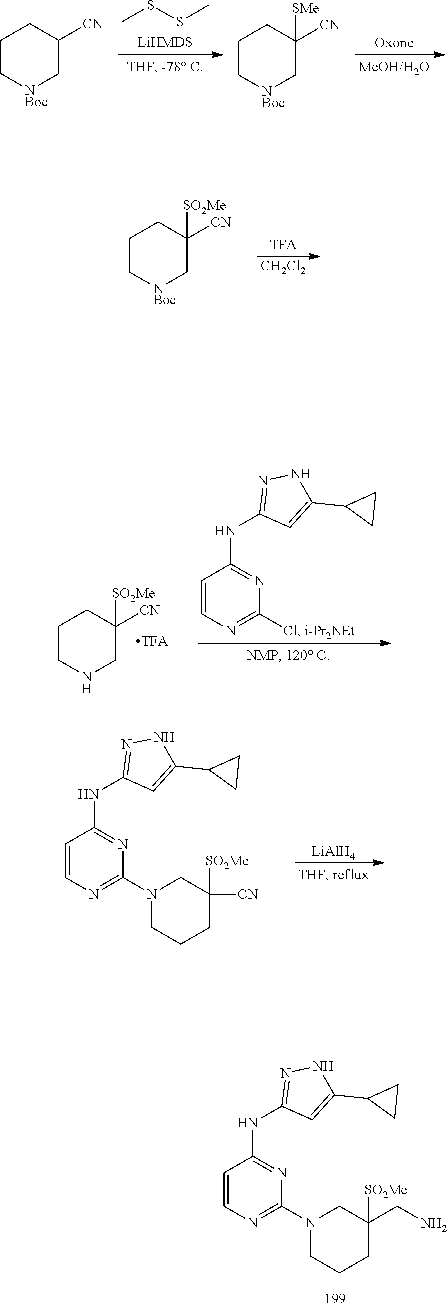

### 2-[3-(Aminomethyl)-3-methylsulfonyl-1-piperidyl]-N-(5-cyclopropyl-1H-pyrazol-3-yl)pyrimidin-4-amine

To a stirred solution of 1-[4-[(5-cyclopropyl-1H-pyrazol-3-yl)amino]pyrimidin-2-yl]-3-methylsulfonyl-piperidine-3-carbonitrile (480 mg, 1.24 mmol) in diethyl ether (1 mL) was added a 2.4 M solution of lithium aluminum hydride in THF (2.6 mL, 6.19 mmol) at 21° C. The reaction mixture was left for stirring overnight at room temperature. Reaction mixture was quenched with water (0.5 mL) followed by addition of NaOH solution (10%, 2.5 mL). After stirring for 2 hours at room temperature, reaction mixture was extracted with dichloromethane (3×15 mL). Combined organic layers were dried over sodium sulfate and concentrated under reduced pressure to get yellow oil. The residue was purified by flash column chromatography, over silica gel, eluting with 88:12 v/v dichloromethane:methanol. The residue was further purified by reversed-phase basic prepHPLC to afford the title compound as a solid (40 mg, 0.102 mmol, 8%).

¹H NMR (400 MHz, Chloroform-d) δ 7.87 (d, J=5.7 Hz, 1H), 6.07 (d, J=5.8 Hz, 2H), 5.95 (s, 1H), 5.04-4.94 (m, 1H), 4.62 (d, J=13.3 Hz, 1H), 3.41-3.29 (m, 1H), 3.14-3.00 (m, 2H), 3.00 (s, 3H), 2.93-2.79 (m, 1H), 2.23-2.09 (m, 1H), 1.88-1.70 (m, 2H), 1.69-1.53 (m, 1H), 0.96-0.83 (m, 2H), 0.74-0.63 (m, 2H).

UPLC MS (basic method, 4 min): Rt=1.15 min, m/z=392.2 [M+H⁺].

### Example 124: Synthesis of Compounds 200-1 and 200-2

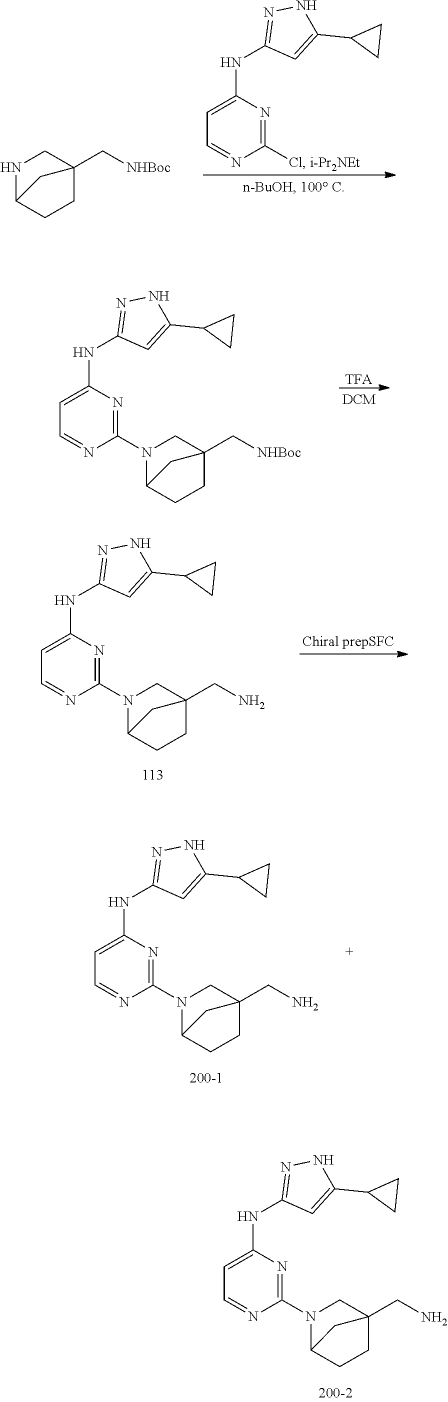

Enantiomer separation by chiral prepSFC was carried out by Reach Separations using a Chiralpak IG (21 mm×250 mm, 5 μm) column and the following parameters: temperature: 40° C.; flow rate: 50 mL/min; mobile phase: 30:70 EtOH:CO₂ (0.2% v/v NH₃), to afford 211-1 as a solid (31.3 mg, 93 μmol) and 211-2 as a solid (32.3 mg, 98 μmol).

### Compound 200-1

¹H NMR (400 MHz, DMSO-d₆) δ 11.89 (s, 1H), 9.31 (s, 1H), 7.80 (d, J=5.7 Hz, 1H), 6.16 (s, 2H), 4.49 (s, 1H), 3.13-3.09 (m, 1H), 2.84-2.77 (m, 2H), 1.90-1.29 (m, 8H), 0.95-0.87 (m, 2H), 0.69-0.60 (m, 2H).

UPLC-MS (4 min, basic): rt=1.17 min, m/z=326.2 [M+H]⁺.

Chiral HPLC (30 min): Chiralpak IG (4.6 mm×250 mm, 5 m) column; mobile phase: 30:70 EtOH:CO₂ (0.1% v/v NH₃), flow rate: 3 mL/min; temperature: 40° C.; rt=19.48 min (major), 22.01 min (minor), >98% ee.

[α]²⁵_{D}: +93.5°

### Compound 200-2

¹H NMR (400 MHz, DMSO-d₆) δ 11.89 (s, 1H), 9.31 (s, 1H), 7.80 (d, J=5.7 Hz, 1H), 6.16 (s, 2H), 4.49 (s, 1H), 3.16-3.08 (m, 1H), 2.83-2.75 (m, 2H), 1.93-1.28 (m, 8H), 0.96-0.87 (m, 2H), 0.71-0.60 (m, 2H).

UPLC-MS (4 min, basic): rt=1.12 min, m/z=326.2 [M+H]⁺.

Chiral HPLC (30 min): Chiralpak IG (4.6 mm×250 mm, 5 m) column; mobile phase: 30:70 EtOH:CO₂ (0.1% v/v NH₃), flow rate: 3 mL/min; temperature: 40° C.; rt=minor enantiomer not detected, 22.36 min (major), >99% ee.

[α]²⁵_{D}: −98.7°

### Example 125: Synthesis of Compound 201

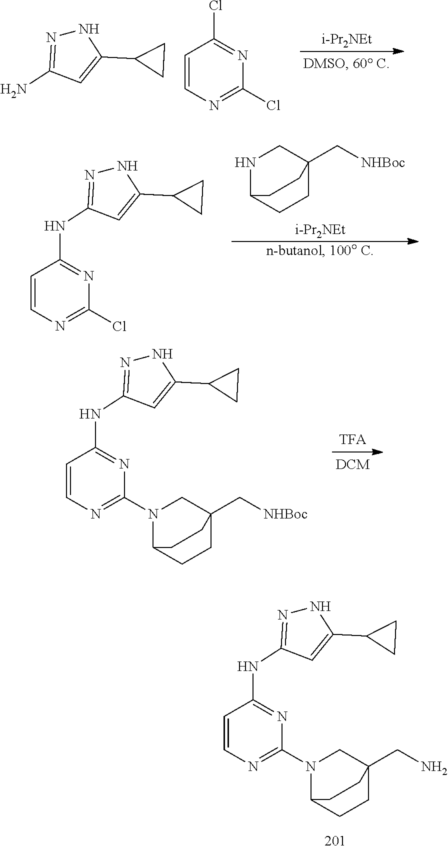

### tert-Butyl N-[[2-[4-[(5-cyclopropyl-1H-pyrazol-3-yl)amino]pyrimidin-2-yl]-2-azabicyclo[2.2.2]octan-4-yl]methyl]carbamate

To 2-chloro-N-(5-cyclopropyl-1H-pyrazol-3-yl)pyrimidin-4-amine (250 mg, 1.06 mmol) and tert-butyl N-(2-azabicyclo[2.2.2]octan-4-ylmethyl)carbamate (306 mg, 1.27 mmol) in n-butanol (12 mL), N,N-diisopropylethylamine (0.46 mL, 2.65 mmol) was added. The mixture was heated to 100° C. and stirred for 24 hours. The solvent was removed under vacuum and the oil was partitioned between DCM (50 mL) and water (50 mL). The organic layer was washed again with water (50 mL) then brine (50 mL) and then dried over MgSO₄. The solvent was removed under vacuum to afford an oil. The crude product was purified by reverse-phase column chromatography, over Cis-modified silica, eluting with a 5% to 95% gradient of MeCN (0.1% v/v NH₃) in water (0.1% v/v NH₃) to afford the title compound as a solid (254 mg, 0.579 mmol, 55%).

¹H NMR (400 MHz, DMSO-d₆) δ 12.07-11.63 (m, 1H), 9.51-9.08 (m, 1H), 8.06-7.63 (m, 1H), 6.89 (s, 1H), 5.98 (s, 1H), 4.60 (d, J=18.4 Hz, 1H), 3.29-3.17 (m, 2H), 2.93-2.76 (m, 2H), 2.04-1.81 (m, 1H), 1.69 (s, 4H), 1.38 (s, 14H), 0.91 (d, J=8.2 Hz, 2H), 0.65 (d, J=22.7 Hz, 2H).

UPLC-MS (2 min, basic method) Rt: 1.11 min, m/z=440.4 [M+H]⁺.

### 2-[4-(Aminomethyl)-2-azabicyclo[2.2.2]octan-2-yl]-N-(5-cyclopropyl-1H-pyrazol-3-yl)pyrimidin-4-amine

To tert-butyl N-[[2-[4-[(5-cyclopropyl-1H-pyrazol-3-yl)amino]pyrimidin-2-yl]-2-azabicyclo[2.2.2]octan-4-yl]methyl]carbamate (120 mg, 0.262 mmol) in DCM (2.5 mL) at 0° C., trifluoroacetic acid (0.20 mL, 2.6 mmol) was added dropwise. The mixture was allowed to warm to room temperature and was stirred for 4 hours. The solvent was removed under vacuum to afford an oil which was dissolved in MeOH and loaded onto an SCX-2 cartridge. The cartridge was washed with MeOH and then the product was eluted with 2M NH₃ in MeOH. The eluent was concentrated under vacuum to afford the title compound as a solid (86 mg, 0.255 mmol, 97%).

¹H NMR (400 MHz, DMSO-d₆) δ 11.92 (s, 1H), 9.67-8.78 (m, 1H), 8.04-7.48 (m, 1H), 6.66-5.52 (m, 2H), 4.62 (s, 1H), 4.11 (s, 1H), 3.51-3.28 (m, 2H, overlapping with water peak), 3.28-3.10 (m, 2H), 2.46-2.34 (m, 1H), 1.95-1.11 (m, 9H), 1.09-0.79 (m, 2H), 0.73-0.50 (m, 2H).

UPLC-MS (2 min, basic method) Rt: 1.25 min, m/z=340.2 [M+H]+.

### Example 126: Synthesis of Compound 14

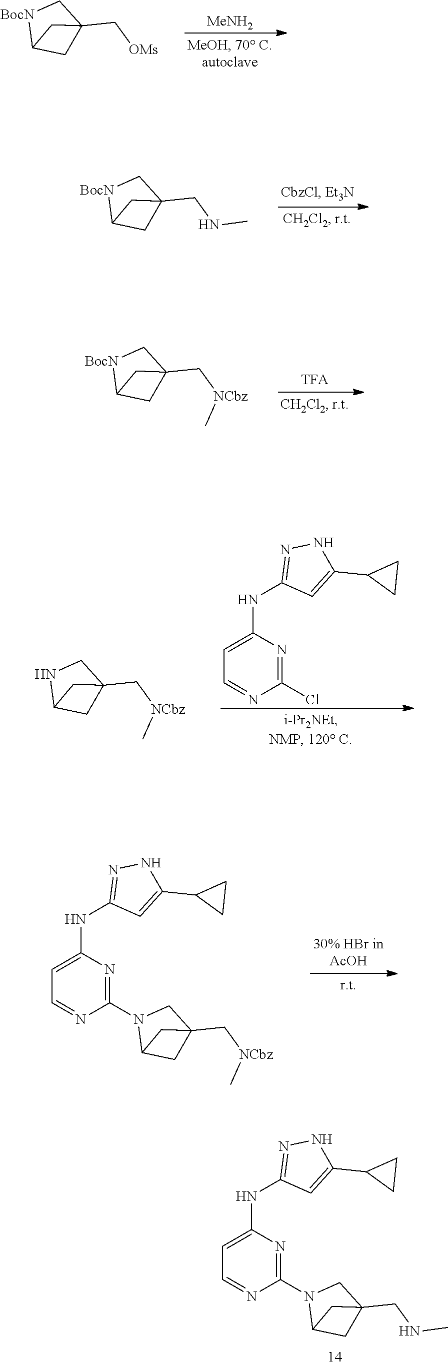

### tert-Butyl 4-(methylaminomethyl)-2-azabicyclo[2.1.1]hexane-2-carboxylate

A solution of 2-methyl-2-propanyl 4-{[(methylsulfonyl)oxy]methyl}-2-azabicyclo[2.1.1]hexane-2-carboxylate (25.0 g, 85.8 mmol) in a 40% solution of methylamine in water (83 mL, 995 mmol) was heated to 70° C. in a sealed 1 Lt autoclave, and stirred for 16 hours. The reaction mixture was cooled to room temperature and concentrated to dryness. The residue was suspended in an aqueous 0.5 M NaOH solution (600 mL) then extracted with EtOAc (3×450 mL). The combined organic extracts were dried (Na₂SO₄), then concentrated to dryness under reduced pressure to afford the title compound as an oil (19.4 g, 85.7 mmol, quant. yield).

¹H NMR (400 MHz, DMSO-d₆) δ 4.15 (t, J=1.8 Hz, 1H), 3.11-3.05 (m, 2H), 2.67 (s, 2H), 2.27 (s, 3H), 1.72 (m, 2H), 1.39 (s, 9H), 1.42-1.31 (m, 1H), 1.29 (m, 2H).

UPLC MS (CAN, 4 min, basic): rt=1.35 min, m/z=227.2 [M+H]⁺100%.

### tert-Butyl 4-[[benzyloxycarbonyl(methyl)amino]methyl]-2-azabicyclo[2.1.1]hexane-2-carboxylate

To a solution of tert-butyl 4-(methylaminomethyl)-2-azabicyclo[2.1.1]hexane-2-carboxylate (19.4 g, 85.7 mmol) and triethylamine (18 mL, 129 mmol) in CH₂Cl₂ (195 mL) in an ice bath cooled to 0° C. was added benzyl chloroformate (14.2 mL, 94.3 mmol) dropwise and allowed to mildly exotherm to 15° C. The reaction was stirred at 25° C. for 3 h then diluted with water (200 mL), followed with CH₂Cl₂ (100 mL) and the organics separated. The aqueous was further extracted with CH₂Cl₂ (2×100 mL). The combined organic extracts were washed with brine (200 mL) and dried (Na₂SO₄) and concentrated to dryness. The residue was purified by column chromatography over silica eluting with a gradient of EtOAc (20% to 60% v/v) in iso-hexane to afford the title compound as an oil (26.50 g, 71.3 mmol, 83% yield).

¹H NMR (400 MHz, CDCl₃) δ 7.37-7.23 (m, 5H), 5.08 (d, J=9.0 Hz, 2H), 4.40-4.15 (m, 1H), 3.49 (d, J=16.0 Hz, 2H), 3.22-3.10 (m, 2H), 2.93 (d, J=6.3 Hz, 3H), 1.85-1.57 (m, 2H), 1.43 (s, 9H), 1.41-1.34 (m, 2H).

UPLC-MS (2 min basic): Rt: 1.24 min, m/z=361.3 [M+H⁺].

### Benzyl N-(2-azabicyclo[2.1.1]hexan-4-ylmethyl)-N-methyl-carbamate

To a solution of tert-butyl 4-[[benzyloxycarbonyl(methyl)amino]methyl]-2-azabicyclo[2.1.1]hexane-2-carboxylate (26.5 g, 71.3 mmol) in CH₂Cl₂ (300 mL) was added trifluoroacetic acid (109 mL, 1.43 mol) at 0° C. The reaction mixture was warmed to room temperature and stirred for 1.5 hours then concentrated under reduced pressure. The residue was taken up in MeOH and purified by SCX-2 cartridge, washing with MeOH and eluting with 2 M NH₃ in MeOH to afford the title compound as an oil (15.8 g, 60.7 mmol, 85% yield).

¹H NMR (400 MHz, CDCl₃) δ 7.34-7.21 (m, 5H), 5.06 (d, J=10.0 Hz, 2H), 3.63-3.55 (m, 1H), 3.50 (d, J=15.1 Hz, 2H), 2.91 (d, J=5.9 Hz, 3H), 2.79-2.64 (m, 2H), 2.32-2.24 (m, 1H), 1.74-1.46 (m, 2H), 1.37-1.07 (m, 2H).

UPLC MS (2 min, basic) rt=0.87 min m/z=261.2 [M+H⁺].

### Benzyl N-[[2-[4-[(5-cyclopropyl-1H-pyrazol-3-yl)amino]pyrimidin-2-yl]-2-azabicyclo[2.1.1]hexan-4-yl]methyl]-N-methyl-carbamate

To a solution of 2-chloro-N-(5-cyclopropyl-1H-pyrazol-3-yl)pyrimidin-4-amine (15.0 g, 63.6 mmol) and benzyl N-(2-azabicyclo[2.1.1]hexan-4-ylmethyl)-N-methyl-carbamate (19.88 g, 76.4 mmol) in NMP (150 mL) was added N,N-diisopropylethylamine (28.0 mL, 159 mmol). The reaction mixture was heated to 120° C. and stirred for 16 hours under a nitrogen atmosphere. The reaction mixture was cooled to room temperature and poured into ice water (1000 mL) and stirred for 1 hour followed by 10 min of sonication. The aqueous layer was decanted, and the residue was dissolved in CH₂Cl₂ (300 mL) and concentrated under reduced pressure. The residue was purified by column chromatography eluting with EtOAc/EtOH/NH₄₀H (3:1:0.02, v/v, 0% to 100%, v/v) and EtOAc (0-100%) to afford the title compound as a solid. (21.2 g, 44.7 mmol, 70% yield).

¹H NMR (400 MHz, CDCl₃) δ 7.95 (d, J=5.7 Hz, 1H), 7.38-7.25 (m, 5H), 6.09 (s, 2H), 5.11 (d, J=6.2 Hz, 2H), 4.77 (d, J=14.2 Hz, 1H), 3.74-3.53 (m, 2H), 3.44 (d, J=17.8 Hz, 2H), 2.97 (d, J=8.3 Hz, 3H), 1.89-1.79 (m, 3H), 1.60-1.44 (m, 2H), 0.97-0.87 (m, 2H), 0.76-0.66 (m, 2H).

UPLC MS (2 min, basic): Rt: 1.09 min, m/z=460.3 [M+H⁺].

### N-(5-Cyclopropyl-1H-pyrazol-3-yl)-2-[4-(methylaminomethyl)-2-azabicyclo[2.1.1]hexan-2-yl]pyrimidin-4-amine

To benzyl N-[[2-[4-[(5-cyclopropyl-1H-pyrazol-3-yl)amino]pyrimidin-2-yl]-2-azabicyclo[2.1.1]hexan-4-yl]methyl]-N-methyl-carbamate (21.00 g, 45.7 mmol) was added 30% HBr in AcOH (400 mL, 457 mmol) and the reaction mixture stirred for 30 mins. The reaction mixture was diluted with water (2000 mL), and washed with CH₂Cl₂ (2×600 mL). The aqueous layer was dropped onto saturated sodium bicarbonate suspension (2000 mL) and the mixture was stirred for 1 hour and stirred. The aqueous layer was extracted with CH₂Cl₂ (3×600 mL) and dried (Na₂SO₄) and concentrated under reduced pressure. The obtained material was purified by column chromatography using 2 M NH₃ in CH₂Cl₂ (0% to 15%) in CH₂Cl₂ to afford the title compound as a solid (8.50 g, 26.1 mmol, 57.16% yield).

Two fractions of the purification above were combined to get 2.1 g of impure material which was further purified by column chromatography, eluting with MeOH (0% to 3% v/v) in CH₂Cl₂ to afford the title compound as a solid (1.90 g, 5.78 mmol, 93.30% yield).

¹H NMR (400 MHz, CD₃OD) δ 7.80 (d, J=5.9 Hz, 1H), 6.13 (d, J=5.8 Hz, 1H), 6.05 (s, 1H), 4.77-4.72 (m, 1H), 3.40 (s, 2H), 2.85 (s, 2H), 2.41 (s, 3H), 1.96-1.81 (m, 3H), 1.54-1.44 (m, 2H), 1.00-0.87 (m, 2H), 0.74-0.64 (m, 2H).

UPLC MS (4 min, basic): Rt: 1.21 min, m/z=326.2 [M+H⁺].

### Example 127: Synthesis of Compound 163

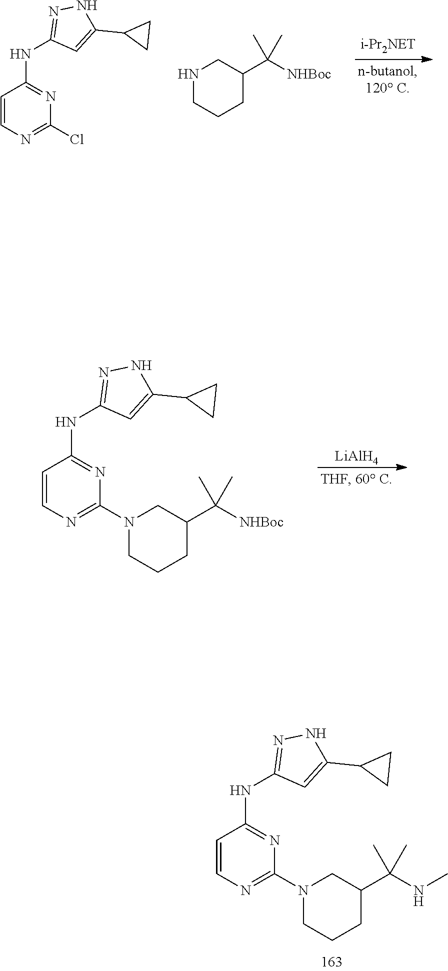 [SMILES: Clc1nccc(Nc2cc(C3CC3)[nH]n2)n1 ; CC(C)(C)OC(=O)NC(C)(C)C1CCCNC1 ; CC(C)(C)OC(=O)NC(C)(C)C1CCCN(c2nccc(Nc3cc(C4CC4)[nH]n3)n2)C1 ; "LiAlH4" ; CNC(C)(C)C1CCCN(c2nccc(Nc3cc(C4CC4)[nH]n3)n2)C1]

### N-(5-Cyclopropyl-1H-pyrazol-3-yl)-2-[3-[1-methyl-1-(methylamino)ethyl]-1-piperidyl]pyrimidin-4-amine

To a stirred solution of tert-butyl N-[1-[1-[4-[(5-cyclopropyl-1H-pyrazol-3-yl)amino]pyrimidin-2-yl]-3-piperidyl]-1-methyl-ethyl]carbamate (150 mg, 0.329 mmol) in THF (3 mL) was added a 2.4 M solution of lithium aluminum hydride in THF (1.4 mL, 3.29 mmol). The reaction mixture was heated to 60° C. and stirred for three hours. The reaction mixture was cooled to 0° C., then quenched with water (0.2 mL) followed by 10% w/v aqueous NaOH solution (0.4 mL). After 20 minutes of stirring, MgSO₄ (500 mg) was added. The suspension was stirred for 16 hours at room temperature. The reaction mixture was filtered through celite, then the celite pad washed with dichloromethane (20 mL), followed by methanol (10 mL). The filtrate was concentrated to dryness under reduced pressure. The crude residue was dissolved in methanol (2 mL), then loaded onto an SCX-2 cartridge. The cartridge was washed with MeOH (20 mL), then the compound was eluted with 2 M ammonia in MeOH (20 mL) The eluent was concentrated to dryness under reduced pressure. The residue was purified by reversed-phase basic prepHPLC to afford the title compound as a solid (40 mg, 0.11 mmol, 34%).

¹H NMR (400 MHz, Chloroform-d) δ 7.97 (d, J=5.7 Hz, 1H), 7.42 (s, 1H), 6.06 (d, J=5.7 Hz, 1H), 5.92 (s, 1H), 4.88 (d, J=12.8 Hz, 1H), 4.68 (d, J=13.0 Hz, 1H), 2.79-2.67 (m, 1H), 2.63-2.52 (m, 1H), 2.34 (s, 3H), 1.89-1.74 (m, 3H), 1.62-1.42 (m, 2H), 1.37-1.20 (m, 1H), 1.11 (s, 3H), 1.07 (s, 3H), 1.03-0.87 (m, 2H), 0.76-0.67 (m, 2H).

UPLC MS (Basic method, 6 min), rt=2.20 min, m/z=356.2 [M+H]⁺.

### Example 128: Synthesis of Compound 160

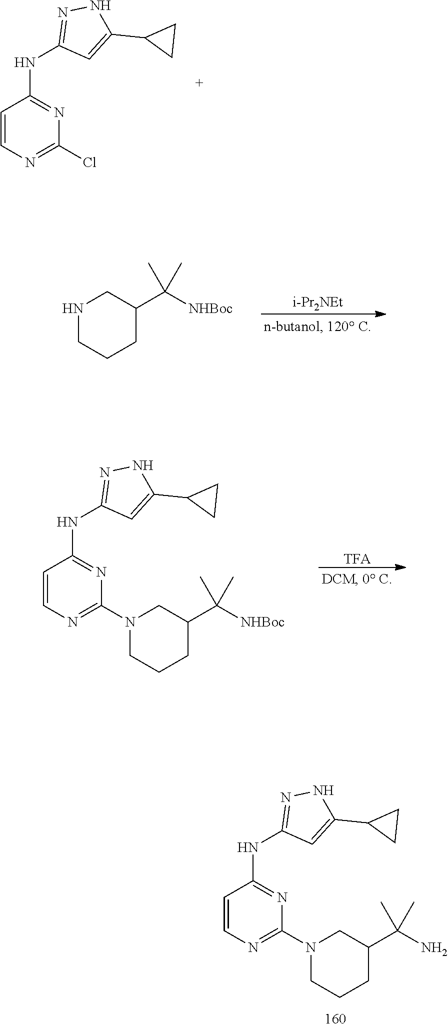 [SMILES: "i-Pr2NEt" ; Clc1nccc(Nc2cc(C3CC3)[nH]n2)n1 ; CC(C)(C)OC(=O)NC(C)(C)C1CCCNC1 ; CC(C)(C)OC(=O)NC(C)(C)C1CCCN(c2nccc(Nc3cc(C4CC4)[nH]n3)n2)C1 ; "TFA" ; CC(C)(N)C1CCCN(c2nccc(Nc3cc(C4CC4)[nH]n3)n2)C1]

### tert-Butyl N-[1-[1-[4-[(5-cyclopropyl-1H-pyrazol-3-yl)amino]pyrimidin-2-yl]-3-piperidyl]-1-methyl-ethyl]carbamate

To a stirred solution of tert-butyl (2-(piperidin-3-yl)propan-2-yl)carbamate (309 mg, 1.27 mmol) and 2-chloro-N-(5-cyclopropyl-1H-pyrazol-3-yl)pyrimidin-4-amine (250 mg, 1.06 mmol) in 1-butanol (2.5 mL) was added N,N-diisopropylethylamine (0.55 mL, 3.2 mmol). The reaction mixture was heated to reflux and stirred for 24 hours. Afterwards, reaction mixture cooled to room temperature and volatiles were removed under reduced pressure. The reaction mixture was diluted with water (10 mL) and extracted with dichloromethane (2×20 mL). The combined organic extracts were washed with a saturated aqueous brine solution (10 mL), dried over Na₂SO₄, and concentrated under reduced pressure to afford the title compound as an oil (365 mg, 0.802 mmol, 76%).

¹H NMR (400 MHz, Chloroform-d) δ 7.90 (d, J=5.7 Hz, 1H), 6.19-5.63 (m, 2H), 4.79 (d, J=13.4 Hz, 1H), 4.63 (d, J=13.8 Hz, 1H), 2.75-2.61 (m, 1H), 2.60-2.49 (m, 1H), 1.90-1.69 (m, 3H), 1.53-1.46 (m, 1H), 1.38 (s, 9H), 1.38-1.31 (m, 2H), 1.30 (s, 3H), 1.25 (s, 3H), 0.89-0.83 (m, 2H), 0.72-0.63 (m, 2H).

UPLC-MS (basic method, 2 min), rt=1.16 min, m/z=442.4 [M+H]⁺.

### 2-[3-(1-Amino-1-methyl-ethyl)-1-piperidyl]-N-(5-cyclopropyl-1H-pyrazol-3-yl)pyrimidin-4-amine

To a stirred solution of tert-butyl N-[1-[1-[4-[(5-cyclopropyl-1H-pyrazol-3-yl)amino]pyrimidin-2-yl]-3-piperidyl]-1-methyl-ethyl]carbamate (150 mg, 0.329 mmol) in dichloromethane (1.5 mL) was added trifluoroacetic acid (0.25 mL, 3.3 mmol) dropwise at 0° C. The reaction mixture was stirred for 16 hours at the room temperature. The volatiles were removed under reduced pressure to afford an oil. The crude residue was dissolved in methanol (2 mL) and loaded onto pre-wet SCX-2 cartridge (1 g). The cartridge was washed with methanol (10 mL) then the compound was eluted with 2 M ammonia in methanol. The eluent was concentrated to dryness under reduced pressure to afford the title compound as a solid (79 mg, 0.23 mmol, 70%).

¹H NMR (400 MHz, Chloroform-d) δ 8.07-7.75 (m, 1H), 6.06-5.99 (m, 1H), 5.92 (s, 1H), 5.00-4.90 (m, 1H), 4.67 (d, J=13.0 Hz, 1H), 2.82-2.66 (m, 1H), 2.63-2.52 (m, 1H), 1.95 (s, 1H), 1.88-1.69 (m, 2H), 1.58-1.46 (m, 1H), 1.38-1.21 (m, 2H), 1.13 (s, 3H), 1.12 (s, 3H), 1.00-0.85 (m, 2H), 0.74-0.65 (m, 2H).

UPLC MS (basic method, 4 min), rt=1.35 min, m/z=342.2 [M+H⁺].

### Example 129: Synthesis of Compound 202

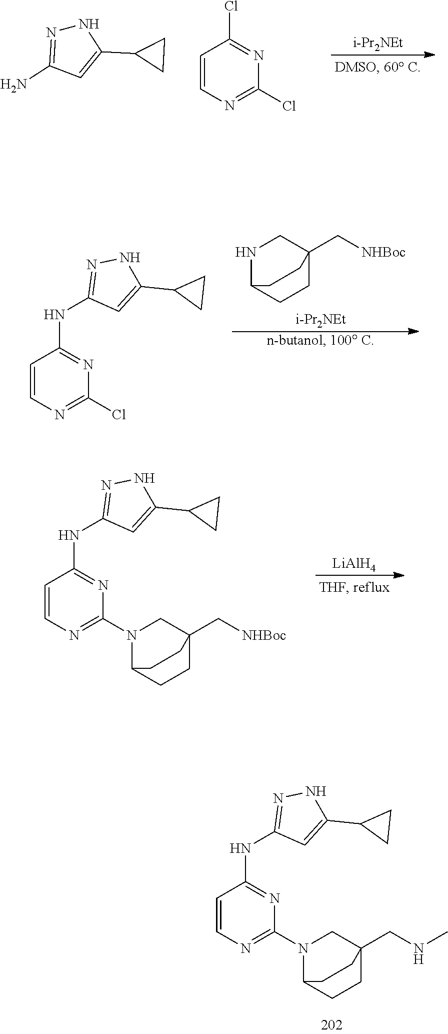

### N-(5-Cyclopropyl-1H-pyrazol-3-yl)-2-[4-(methylaminomethyl)-2-azabicyclo[2.2.2]octan-2-yl]pyrimidin-4-amine

To tert-butyl N-[[2-[4-[(5-cyclopropyl-1H-pyrazol-3-yl)amino]pyrimidin-2-yl]-2-azabicyclo[2.2.2]octan-4-yl]methyl]carbamate (74 mg, 0.17 mmol) in anhydrous THF (6 mL) under a nitrogen atmosphere was added a 2.4 M solution of lithium aluminium hydride in THF (0.70 mL, 1.7 mmol) at 0° C. The reaction mixture was then heated to 55° C. for and stirred for 4.5 hours. The mixture was cooled to 0° C. and then quenched with the addition of water (160 μL), 15% w/v NaOH (aq) (160 μL) and water (470 μL). The mixture was warmed to room temperature and stirred for 10 minutes. MgSO₄ (ca. 1 g) was added and the mixture was stirred for a further 10 minutes. The mixture was the filtered through a pad of celite which was washed with DCM (10 mL) followed by EtOH (10 mL). The filtrate was concentrated to dryness under reduced pressure, then the residue was purified by reversed-phase prepHPLC. The residue was dissolved in MeOH (2 mL), then loaded onto an SCX-2 cartridge. The cartridge was washed with MeOH (10 mL), then the compound was eluted with 2 M NH₃ in MeOH (10 mL). The eluent was concentrated to dryness under reduced pressure to afford the title compound as a white solid (31 mg, 86 μmol, 51%).

¹H NMR (400 MHz, DMSO-d₆) δ 11.92 (s, 1H), 9.56-9.01 (m, 1H), 8.01-7.58 (m, 1H), 6.58-5.77 (m, 2H), 4.62 (s, 1H), 3.31-3.20 (m, 2H), 2.41-2.17 (m, 5H), 1.91-1.57 (m, 5H), 1.57-1.34 (m, 4H), 0.98-0.82 (m, 2H), 0.64 (s, 2H).

UPLC-MS (4 min, basic method) Rt: 1.38 min, m/z=354.2 [M+H]⁺.

### Example 129: Degradation of MycC/MycN Protein

For L363 cells (suspension), 1E6 cells were plated into each well of a 6 well plate. For SK-N-BE(2) cells (adherent), 5E5 cells were plated into each well of a 6 well dish. Cells were cultured for 24 hrs then treated with exemplary compounds at 0.1, 0.5, 1.0, 3.0 and 6.0 mM final assay concentrations plus DMSO control. All compounds were diluted to 10 mM in DMSO. Cells were treated with the compound for 24 hrs, then for both cell types, media containing cells were removed from wells into a 15 ml centrifuge tube, and remaining adherent SK-N-BE(2) cells were also scraped from the wells into the appropriate tubes. Tubes were centrifuged, cells were washed with PBS then re-centrifuged to pellet cells. A RIPA buffer cocktail was added to the cells on ice for 5 mins. Cell lysates were clarified by centrifugation and stored at −80 degrees until required. BCA assay was performed on each lysate to determine the protein concentration.

For validation studies of antibodies, lysates and antibodies were used at a number of concentrations (Lysates: 2, 1, 0.2 mg/ml; Antibodies: 1 in 50, 1 in 200 dilution). Once an appropriate lysate concentration and antibody concentration was determined, samples were screened in the JESS technology (Protein Simple, San Jose, CA—https://www.proteinsimple.com/jess.html).

Target protein antibodies (n-myc and c-myc) were detected in the chemi-luminescence channel and loading controls (tubulin and GAPDH) were detected using a Near Infra Red (NIR) labeled secondary antibody.

### Cell Proliferation/Viability Measures

For SK-N-EB2 adherent cells, 5E5 cells in 1.9 mls media were plated into 6 well dishes and incubated for 24 hrs. Exemplary compounds were added (100 ul, 1 in 20 dilution in media) to each well (final assay concentration 6, 3, 1 and 0.5 mM) together with DMSO control wells and incubated for 24 hrs. Cells were scrapped off the plate, centrifuged, washed with PBS, centrifuged, then a RIPA buffer cocktail added to the cells (100 ul), centrifuged and stored at −80 for later analysis. For L363 suspension cells, 1E6 cells were plated into 24 well dishes (950 ul media) and incubated for 24 hrs. Exemplary compounds were added (50 ul, 1 in 20 dilution in media) to each well (final assay concentration 6, 3, 1 and 0.5 mM) together with DMSO control wells and incubated for 24 hrs. Cells were aspirated into tubes, centrifuged, washed with PBS, centrifuged, then a RIPA buffer cocktail added to the cells (100 ul), centrifuged and stored at −80 for later analysis.

For Western blot, a BCA (total protein) assay was run on all samples, these were then run in batches of 5 compounds plus control (DMSO) on each Western blot run—n-myc and c-myc were run in separate experiments in the chemiluminescence channel. Both tubulin and GAPDH were run as loading controls in all lanes in the near infra red channel.

For cytotox, both SK-N-EB2 or L363 cells were run in 384 well format. For SK-N-EB2 cells, 5000 cells per well (in 30 ul media) were incubated for 24 hrs prior to the addition of compounds (10 ul, 1 in 4 dilution, 10 mM top final concentration, 1 in 2 dilutions) for 24 hrs. For L363 cells, 2000 cells per well (30 ul media) were incubated for 24 hrs prior to the addition of compounds (10 ul, 10 mM 1 in 2 dilutions) for 24 hrs. In both cases Promega Cell Titer GLO was added according to the manufactures instructions and the plates read immediately in the luminometer.

TABLE 1
Percent Degradation of MycC/MycN Protein by Various Compounds

| | % | % | % | % | % | % | % | % |
|---|---|---|---|---|---|---|---|---|
| | Degradation | Degradation | Degradation | Degradation | Degradation | Degradation | Degradation | Degradation |
| | of MycN | of MycC | of MycN | of MycC | of MycN | of MycC | of MycN | of MycC |
| Cmpd | (SKNBE2) | (L-363) | (SKNBE2) | (L-363) | (SKNBE2) | (L-363) | (SKNBE2) | (L-363) |
| No. | 24 h, 6 μM | 24 h, 6 μM | 24 h, 3 μM | 24 h, 3 μM | 24 h, 1 μM | 24 h, 1 μM | 24 h, 0.5 μM | 24 h, 0.5 μM |
| 1 | *** | ** | * | ND | ND | ND | ND | ND |
| 2 | *** | ** | * | ** | * | ** | * | ** |
| 3 | *** | **** | * | | * | ** | * | * |
| 4 | ** | **** | ** | **** | ** | ** | * | * |
| 5 | ** | ** | ND | ** | ND | * | ND | * |
| 6 | * | ** | * | * | * | ** | * | * |
| 7 | **** | ND | *** | ND | * | * | * | ND |
| 8 | | ND | * | ** | ND | * | ND | * |
| 9 | * | **** | * | ** | ** | ** | ** | ** |
| 10 | **** | ** | **** | ND | ** | * | ND | * |
| 11 | **** | ND | **** | * | **** | ND | * | ND |
| 12 | *** | **** | ** | *** | ND | * | ND | * |
| 13 | **** | ** | **** | * | *** | * | * | ND |
| 14 | **** | **** | ** | **** | ** | **** | * | ** |
| 15 | **** | ND | *** | ** | ** | ND | ** | * |
| 15 | **** | ** | *** | * | ** | ** | ** | * |
| 16 | **** | * | **** | * | **** | * | * | * |
| 17 | **** | **** | *** | * | ** | ND | ** | ND |
| 18 | **** | ** | **** | * | *** | * | ** | * |
| 18 | **** | ** | **** | * | *** | ** | ** | * |
| 19 | ** | ND | ** | ND | ND | ND | ND | * |
| 20 | *** | * | ** | ND | * | ND | * | ND |
| 21 | ** | ** | ** | * | * | ND | ** | ND |
| 22 | *** | **** | ** | * | * | ND | * | ND |
| 23 | **** | **** | **** | **** | *** | ND | ** | ND |
| 24 | **** | **** | **** | **** | *** | ND | ** | * |
| 25 | ** | ND | ** | ND | ** | * | ** | * |
| 26 | *** | ND | ** | * | ** | ** | ** | ** |
| 27 | **** | ** | *** | * | ** | ** | ** | * |
| 28 | **** | *** | **** | ND | **** | * | | * |
| 29 | **** | **** | **** | **** | **** | *** | **** | ** |
| 30 | **** | **** | **** | **** | *** | ** | * | ND |
| 31 | **** | **** | **** | **** | **** | ND | **** | ND |
| 32 | *** | * | *** | ND | ** | * | ** | ND |
| 33 | *** | ND | *** | ND | ** | ND | ** | ND |
| 34 | * | ND | ND | ND | ND | ND | ND | ND |
| 35 | **** | **** | **** | *** | *** | * | ** | ND |
| 36 | **** | **** | **** | * | * | ** | ** | * |
| 37 | **** | ND | *** | * | ** | * | ** | ND |
| 38 | *** | * | ** | * | ** | * | ** | * |
| 39 | ** | ND | ** | ND | * | ** | ND | ND |
| 40 | * | ND | * | * | ND | * | ND | * |
| 40 | * | ND | * | * | ND | * | ND | * |
| 41 | **** | ** | *** | * | ** | * | ** | ** |
| 42 | **** | **** | *** | ND | *** | * | ** | * |
| 43 | *** | * | ** | ** | ** | * | ** | ** |
| 44 | **** | **** | ** | * | * | ND | ND | ND |
| 44 | **** | **** | ** | * | * | ND | ND | ND |
| 45 | **** | **** | **** | **** | **** | **** | **** | **** |
| 46 | * | ND | ND | ND | ND | ND | ND | ND |
| 70 | ** | ND | ND | ND | * | ND | ND | ND |
| 47 | **** | **** | *** | ** | ** | ** | ** | ** |
| 48 | * | ** | * | * | * | ** | * | ND |
| 50 | **** | *** | *** | ** | ** | * | ** | ** |
| 51 | *** | *** | ** | *** | ** | ** | ** | * |
| 52 | * | ND | ND | ND | ND | ND | | ND |
| 53 | * | * | ND | ** | ND | ND | ND | ND |
| 64 | ** | ** | * | ** | ND | * | * | ND |
| 65 | *** | *** | ** | * | * | * | * | * |
| 66 | **** | **** | **** | **** | **** | ND | | * |
| 67 | **** | **** | **** | **** | *** | * | * | * |
| 68 | **** | **** | **** | **** | * | * | * | * |
| 69 | **** | **** | **** | **** | ** | ND | ** | * |
| 71 | **** | **** | **** | **** | *** | **** | ** | ** |
| 72 | **** | **** | *** | ND | ** | ND | ** | ND |
| 73 | **** | **** | *** | ** | ** | ND | * | ND |
| 74 | **** | ND | *** | ND | ** | ND | ** | ND |
| 75 | **** | *** | *** | ND | ** | ND | ** | ND |
| 76 | **** | **** | **** | * | * | ND | ND | ND |
| 78 | **** | **** | **** | *** | ** | * | * | ND |
| 81 | **** | **** | **** | **** | *** | ** | ** | ** |
| 84 | ** | ** | ND | * | | ** | ND | ** |
| 85 | *** | * | ** | ND | * | ND | * | * |
| 86 | **** | **** | **** | **** | ** | ND | ** | ND |
| 101 | ** | ** | * | ** | * | ** | * | ** |
| 102 | ** | ** | * | ** | * | ** | * | ** |
| 103 | **** | **** | **** | ***** | ** | * | * | * |
| 104 | ** | ** | ** | ND | * | * | ND | ND |
| 105 | **** | **** | * | * | * | ND | * | ND |
| 106 | ** | * | ** | * | * | * | * | * |
| 107 | ** | * | ** | * | ** | ND | * | ND |
| 108 | **** | **** | *** | **** | *** | ND | * | ND |
| 109 | **** | **** | *** | **** | *** | * | ** | * |
| 110 | **** | **** | *** | **** | *** | ND | ** | * |
| 111 | *** | * | * | * | * | * | * | * |
| 112 | **** | *** | *** | ND | ND | ND | ND | ND |
| 113 | **** | **** | **** | **** | **** | **** | **** | **** |
| 114 | **** | **** | **** | **** | **** | **** | **** | **** |
| 115 | **** | **** | **** | **** | *** | ND | ** | ND |
| 116 | **** | **** | **** | **** | * | * | * | ND |
| 117 | **** | **** | **** | **** | **** | **** | **** | **** |
| 118 | **** | ND | * | * | ** | * | * | ND |
| 119 | **** | **** | **** | **** | *** | *** | *** | ND |
| 121 | **** | **** | **** | **** | *** | **** | *** | ND |
| 122-1 | **** | **** | ** | **** | ** | *** | ** | ** |
| 122-2 | **** | **** | *** | **** | * | *** | | ** |
| 123-1 | **** | **** | *** | **** | * | ** | | * |
| 123-2 | **** | **** | *** | **** | ** | *** | ** | ** |
| 124 | **** | **** | ** | | ** | * | ** | |
| 125 | **** | **** | *** | | ** | ** | ** | ** |
| 126 | **** | **** | **** | **** | **** | **** | **** | **** |
| 127 | **** | **** | **** | **** | **** | **** | *** | * |
| 159^{a} | **** | **** | **** | **** | **** | **** | **** | ** |
| 159^{b} | **** | **** | *** | ** | ** | ** | ** | ** |
| 161 | **** | **** | **** | **** | **** | **** | *** | * |
| 162^{a} | **** | **** | *** | ** | ** | ** | ** | ** |
| 162^{b} | **** | ** | **** | ND | ** | | ND | ND |
| 177 | *** | **** | *** | ** | * | ** | ** | * |
| 180 | **** | **** | **** | **** | **** | **** | * | *** |
| 181 | **** | **** | ** | ND | * | * | * | ND |
| 182 | **** | **** | **** | ND | ** | ND | ** | ND |
| 183 | **** | **** | **** | **** | *** | ND | ** | ND |
| 184 | **** | **** | **** | *** | *** | ND | ** | ND |
| 185 | **** | **** | **** | **** | **** | **** | **** | **** |
| 186 | **** | **** | **** | **** | * | * | * | ND |
| 187 | *** | *** | ** | ** | ** | ** | ** | * |
| 188 | **** | **** | **** | **** | *** | **** | *** | **** |
| 189 | **** | **** | **** | **** | **** | **** | *** | **** |
| 190 | *** | ND | *** | ND | ** | ND | ** | ND |
| 191 | **** | **** | **** | *** | ** | * | * | ND |

^{a}Diastereomer 1 of the compound was evaluated.
^{b}Diastereomer 2 of the compound was evaluated.
Key:
****: degradation 80-100%
***: degradation 50-79%
**: degradation 20-49%
*: degradation <20%
ND—No Degradation

It will be appreciated that compounds reported as a salt form (e.g., a TFA salt) may or may not have a 1:1 stoichiometry, and/or for example, reported potency concentrations or other assay results may be, e.g., slightly higher or lower.

The practice of the present disclosure will employ, unless otherwise indicated, conventional methods of organic chemistry, protein chemistry, biochemistry, recombinant DNA techniques and pharmacology, within the skill of the art. While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

What is claimed is:

1. A compound of Formula I: 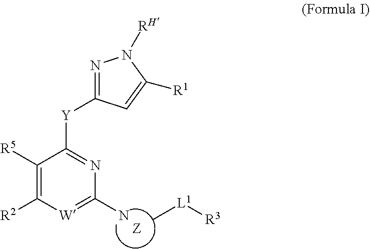
or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CHF, CF₂, CH(CH₃), C(CH₃)₂, and C(CH₂CH₂), CHCF₃, CHCF₂H, CH(OCF₂H), CHOH, C(CH₃)OH, and CH(OCH₃);
ring Z is a 4-10 membered N-bound mono- or bicyclic heterocyclic, wherein Z may optionally be substituted by one or two substituents each independently selected from the group consisting of halo, hydroxyl, C₁-C₄ alkyl (optionally substituted by one, two or three halogens), and oxo, C₁-C₄ alkoxy, and cyano;
L¹ is bond or C₁-C₆ alkylene (optionally substituted by one, two or three halogens, R⁴);
R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
R⁴ is each independently selected from the group consisting of halogen and C₁-C₆ alkyl, or two R⁴ together with the carbon from which the R⁴ are attached form a C₃-C₅ carbocyclyl;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R^{N} is selected from the group consisting of H and C₁-C₃alkyl, or two R^{N} together with the nitrogen atoms to which the R^{N}s are attached form a 4-6 membered heterocyclic ring;
R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
R² is selected from the group consisting of H, F, CF₃, —C(O)—O-methyl, —C(O)OH, —O—methyl, —O-ethyl, C₃-C₇ cycloalkyl, and heterocyclyl; and
R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring;
wherein when ring Z is a 6 membered monocyclic heterocycle, L¹ is C₁-C₆alkylene or L¹ is a bond and R³ is selected from C₂-C₆alkyl, SO₂C₁-C₄alkyl, and NHR′ wherein R′ is selected from the group consisting of C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo.

2. The compound of claim 1, wherein W is N, and having the Formula Ia: 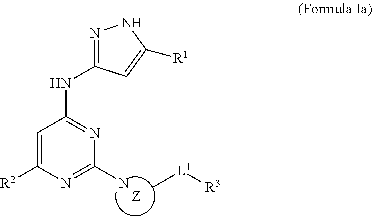
or a pharmaceutically acceptable salt, stereoisomer and/or N-oxide thereof.

3. The compound of claim 1 or 2, wherein R¹ is a 5-6 membered heterocyclyl or C₃-C₆ cycloalkyl.

4. The compound of any one of claims 1-3, wherein R¹ is a C₃-C₆ cycloalkyl.

5. The compound of any one of claims 1-4, wherein R¹ is cyclopropyl.

6. The compound of any one of claims 1-4, wherein R¹ is cyclopentyl.

7. The compound of any one of claims 1-6, wherein ring Z is selected from the group consisting of 4-6 membered monocyclic heterocycle, 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), and oxo.

8. The compound of any one of claims 1-6, wherein ring Z is selected from the group consisting of 6-10 membered spiroheterocycle, a 6-10 membered fused bicyclic heterocyclic, and a 6-10 membered bridged cycloheteroalkyl, wherein ring Z may optionally be substituted by one or two substituents each independently selected from the group consisting of halo, hydroxyl, C₁-C₄ alkyl (optionally substituted by one, two or three halogens), and oxo.

9. The compound of any one of claims 1-6, wherein ring Z is selected from the group consisting of:

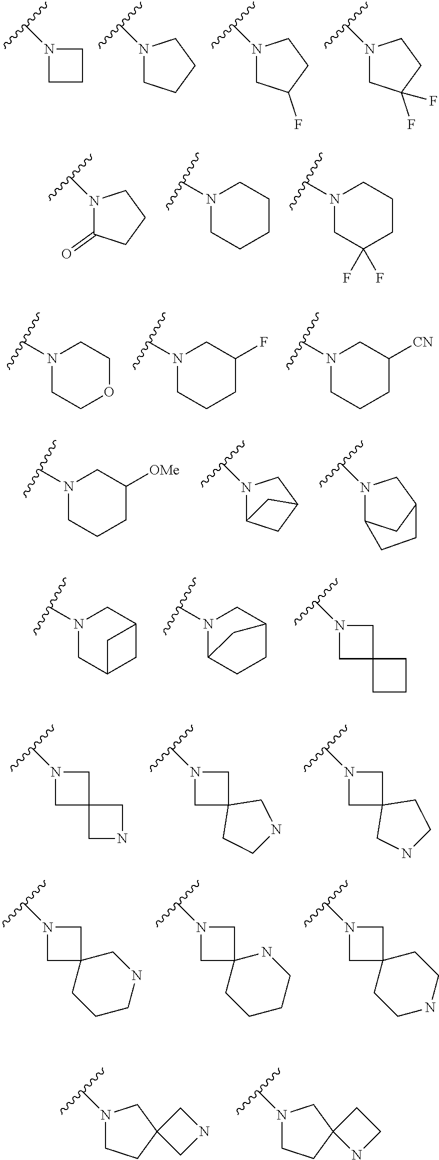

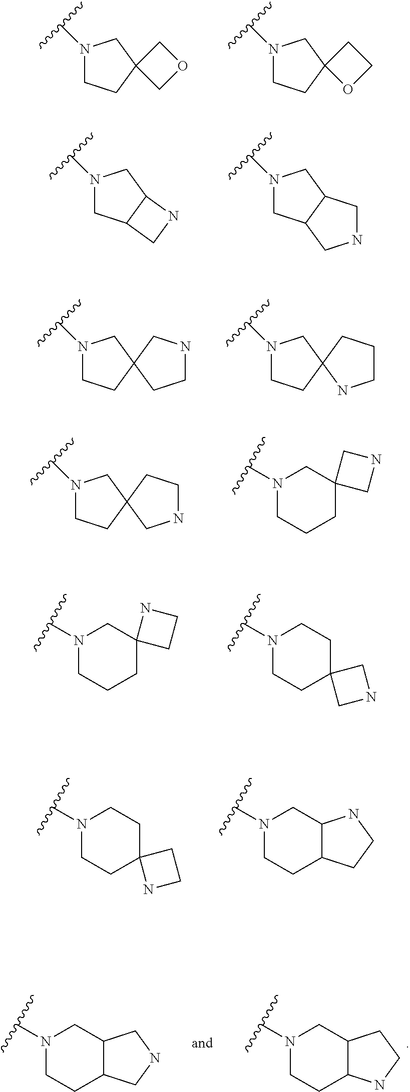

10. The compound of any one of claims 1-9, represented by Formula Ib: 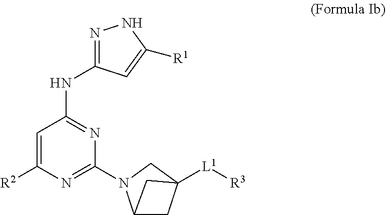
or a pharmaceutically acceptable salt, stereoisomer, and/or N-oxide thereof.

11. The compound of any one of claims 1-9, represented by Formula Ic: 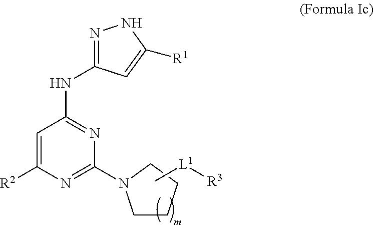
or a pharmaceutically acceptable salt, stereoisomer, and/or N-oxide thereof, wherein m is 0, 1, or 2.

12. The compound of any one of claims 1-9, represented by Formula Id: 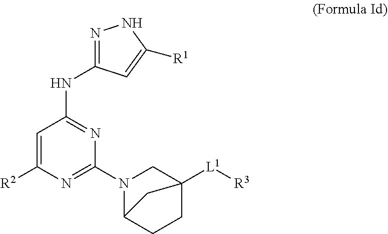
or a pharmaceutically acceptable salt, stereoisomer, and/or N-oxide thereof.

13. The compound of any one of claims 1-9, represented by Formula Ie: 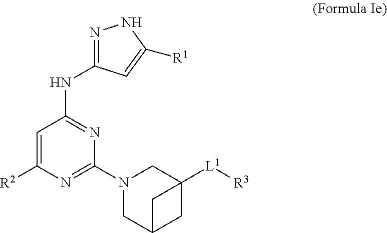
or a pharmaceutically acceptable salt, stereoisomer, and/or N-oxide thereof.

14. The compound of any one of claims 1-13, wherein L¹ is bond.

15. The compound of any one of claims 1-13, wherein L¹ is C₁-C₆ alkylene.

16. The compound of claim 15, wherein L¹ is methylene.

17. The compound of any one of claims 1-13, wherein L¹ is C₁-C₄ alkylene substituted by one or two R⁴.

18. The compound of any one of claims 1-13 and 17, wherein L¹ is methylene substituted by one or two R⁴.

19. The compound of claim 18, wherein R⁴ is C₁-C₃ alkyl (optionally substituted by one, two or three halogens).

20. The compound of claim 18, wherein two R⁴s together with the carbon to which the R⁴ are attached form a C₃-C₄ carbocyclyl.

21. The compound of any one of claims 1-20, wherein R³ is H or C₁-C₆ alkyl.

22. The compound of claim 21, wherein R³ is H or methyl.

23. The compound of any one of claims 1-20, wherein R³ is NR′R′.

24. The compound of claim 23, wherein R³ is NH₂, NH(CH₃), or N(CH₃)₂.

25. The compound of any one of claims 1-20, wherein R³ is 4-6 membered heterocyclyl optionally substituted by one or two substituents each independently selected from the group consisting of halo, hydroxyl, C₁-C₄ alkyl (optionally substituted by one, two or three halogens), and oxo.

26. The compound of any one of claims 1-25, wherein R² is H or F.

27. The compound of any one of claims 1-26, wherein R² is H.

28. The compound of claim 1, wherein R⁵ is H or methyl.

29. The compound of claim 1 or 28, wherein R⁵ is H.

30. A compound selected from the group consisting of: 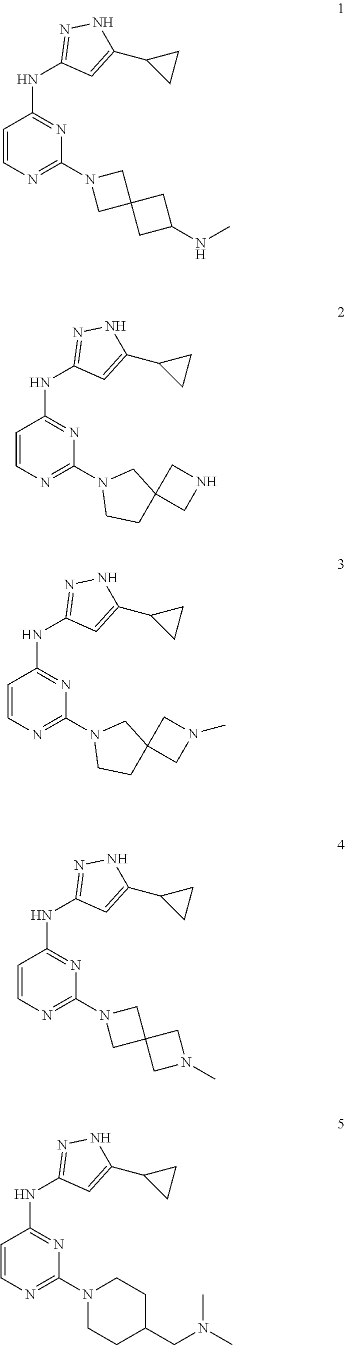 [SMILES: CNC1CC2(C1)CN(c1nccc(Nc3cc(C4CC4)[nH]n3)n1)C2 ; c1cc(Nc2cc(C3CC3)[nH]n2)nc(N2CCC3(CNC3)C2)n1 ; CN1CC2(CCN(c3nccc(Nc4cc(C5CC5)[nH]n4)n3)C2)C1 ; CN1CC2(C1)CN(c1nccc(Nc3cc(C4CC4)[nH]n3)n1)C2 ; CN(C)CC1CCN(c2nccc(Nc3cc(C4CC4)[nH]n3)n2)CC1] 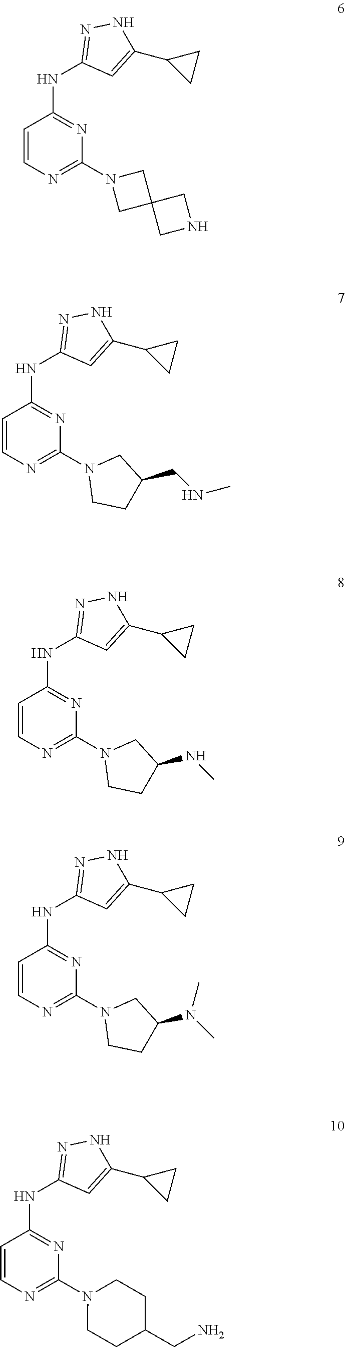 [SMILES: c1cc(Nc2cc(C3CC3)[nH]n2)nc(N2CC3(CNC3)C2)n1 ; CNC[C@H]1CCN(c2nccc(Nc3cc(C4CC4)[nH]n3)n2)C1 ; CN[C@H]1CCN(c2nccc(Nc3cc(C4CC4)[nH]n3)n2)C1 ; CN(C)[C@H]1CCN(c2nccc(Nc3cc(C4CC4)[nH]n3)n2)C1 ; NCC1CCN(c2nccc(Nc3cc(C4CC4)[nH]n3)n2)CC1] 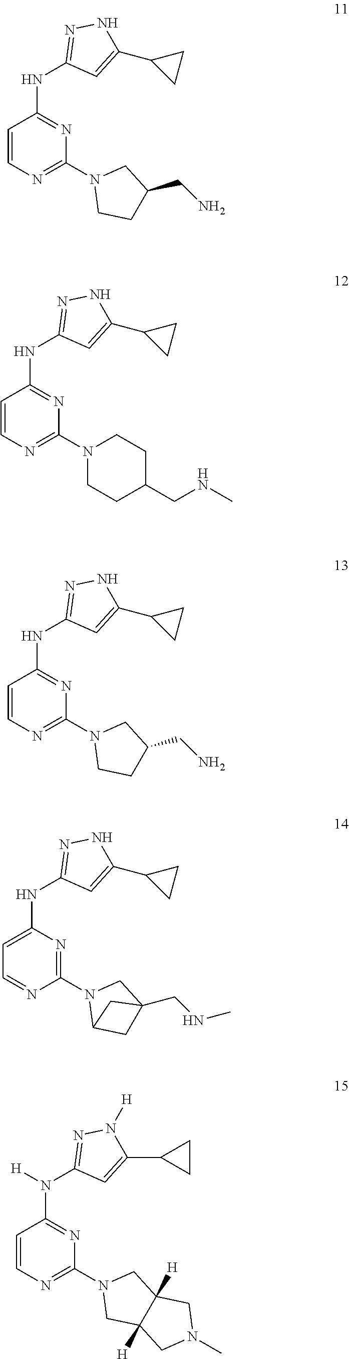 [SMILES: NC[C@H]1CCN(c2nccc(Nc3cc(C4CC4)[nH]n3)n2)C1 ; CNCC1CCN(c2nccc(Nc3cc(C4CC4)[nH]n3)n2)CC1 ; NC[C@@H]1CCN(c2nccc(Nc3cc(C4CC4)[nH]n3)n2)C1 ; CNCC12CC(C1)N(c1nccc(Nc3cc(C4CC4)[nH]n3)n1)C2 ; CN1C[C@@H]2CN(c3nccc(Nc4cc(C5CC5)[nH]n4)n3)C[C@@H]2C1] 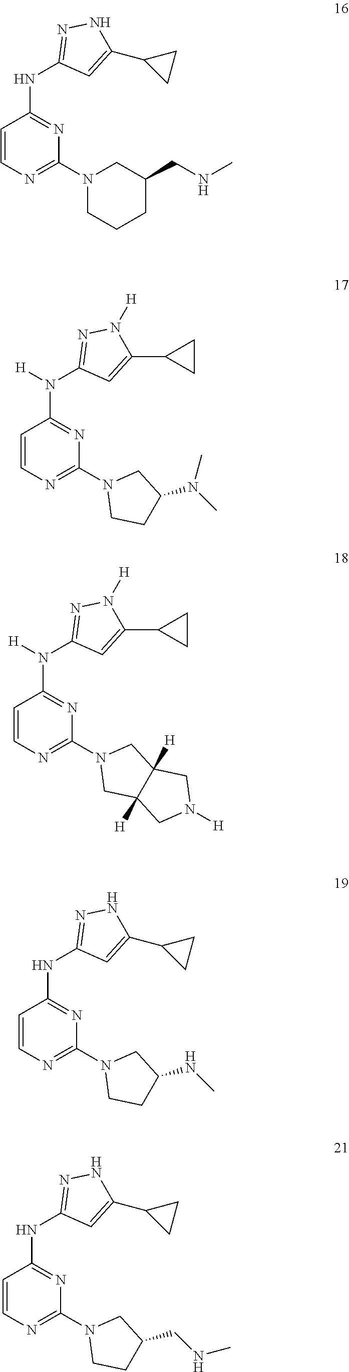 [SMILES: CNC[C@H]1CCCN(c2nccc(Nc3cc(C4CC4)[nH]n3)n2)C1 ; CN(C)[C@@H]1CCN(c2nccc(Nc3cc(C4CC4)[nH]n3)n2)C1 ; c1cc(Nc2cc(C3CC3)[nH]n2)nc(N2C[C@H]3CNC[C@H]3C2)n1 ; CN[C@@H]1CCN(c2nccc(Nc3cc(C4CC4)[nH]n3)n2)C1 ; CNC[C@@H]1CCN(c2nccc(Nc3cc(C4CC4)[nH]n3)n2)C1] 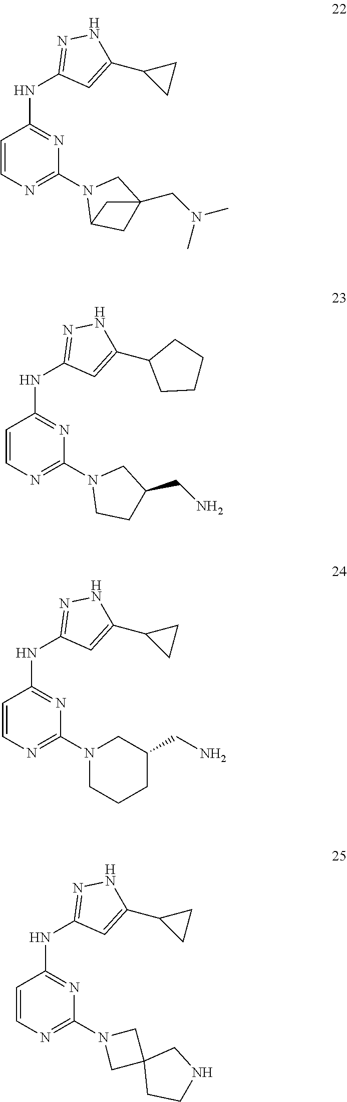 [SMILES: CN(C)CC12CC(C1)N(c1nccc(Nc3cc(C4CC4)[nH]n3)n1)C2 ; NC[C@H]1CCN(c2nccc(Nc3cc(C4CCCC4)[nH]n3)n2)C1 ; NC[C@@H]1CCCN(c2nccc(Nc3cc(C4CC4)[nH]n3)n2)C1 ; c1cc(Nc2cc(C3CC3)[nH]n2)nc(N2CC3(CCNC3)C2)n1] 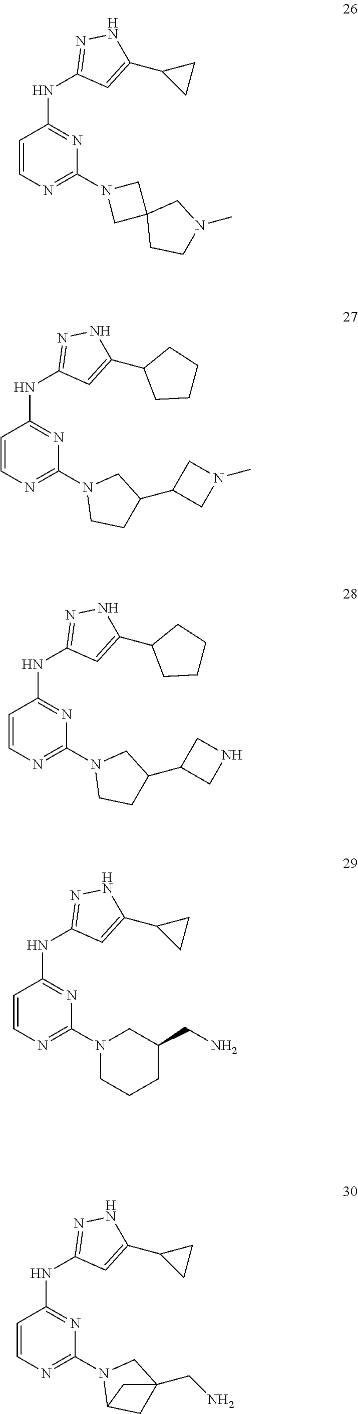 [SMILES: CN1CCC2(C1)CN(c1nccc(Nc3cc(C4CC4)[nH]n3)n1)C2 ; CN1CC(C2CCN(c3nccc(Nc4cc(C5CCCC5)[nH]n4)n3)C2)C1 ; c1cc(Nc2cc(C3CCCC3)[nH]n2)nc(N2CCC(C3CNC3)C2)n1 ; NC[C@H]1CCCN(c2nccc(Nc3cc(C4CC4)[nH]n3)n2)C1 ; NCC12CC(C1)N(c1nccc(Nc3cc(C4CC4)[nH]n3)n1)C2] 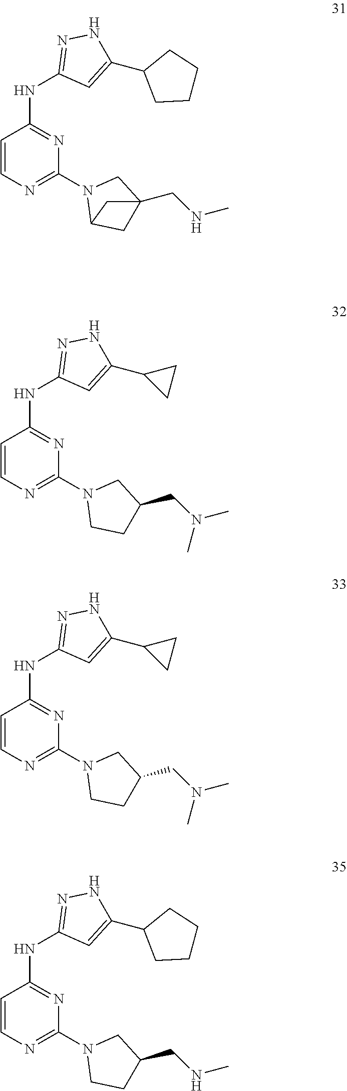 [SMILES: CNCC12CC(C1)N(c1nccc(Nc3cc(C4CCCC4)[nH]n3)n1)C2 ; CN(C)C[C@H]1CCN(c2nccc(Nc3cc(C4CC4)[nH]n3)n2)C1 ; CN(C)C[C@@H]1CCN(c2nccc(Nc3cc(C4CC4)[nH]n3)n2)C1 ; CNC[C@H]1CCN(c2nccc(Nc3cc(C4CCCC4)[nH]n3)n2)C1] 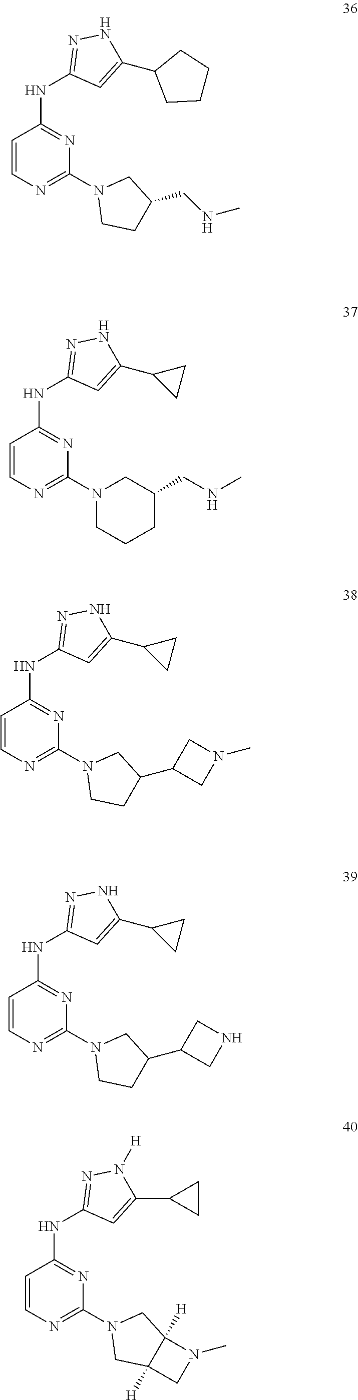 [SMILES: CNC[C@@H]1CCN(c2nccc(Nc3cc(C4CCCC4)[nH]n3)n2)C1 ; CNC[C@@H]1CCCN(c2nccc(Nc3cc(C4CC4)[nH]n3)n2)C1 ; CN1CC(C2CCN(c3nccc(Nc4cc(C5CC5)[nH]n4)n3)C2)C1 ; c1cc(Nc2cc(C3CC3)[nH]n2)nc(N2CCC(C3CNC3)C2)n1 ; CN1C[C@H]2CN(c3nccc(Nc4cc(C5CC5)[nH]n4)n3)C[C@H]21] 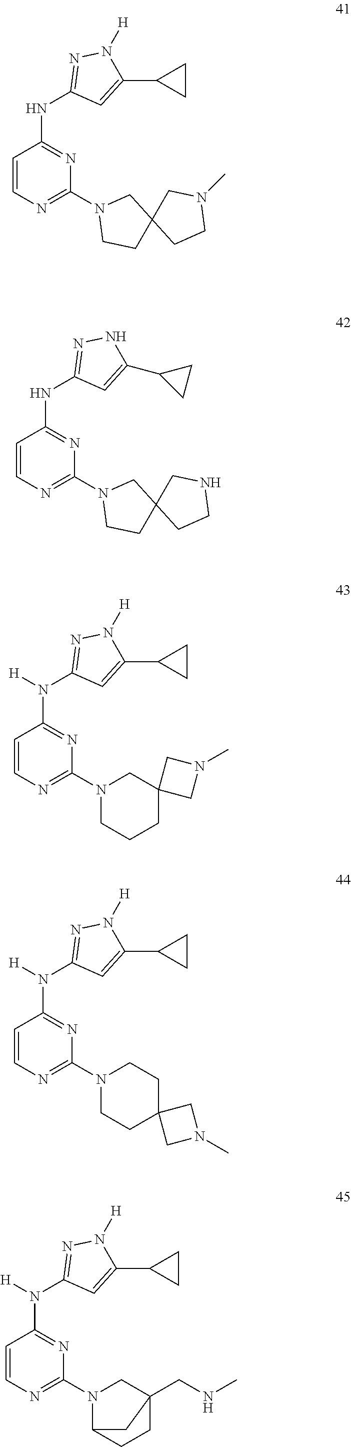 [SMILES: CN1CCC2(CCN(c3nccc(Nc4cc(C5CC5)[nH]n4)n3)C2)C1 ; c1cc(Nc2cc(C3CC3)[nH]n2)nc(N2CCC3(CCNC3)C2)n1 ; CN1CC2(CCCN(c3nccc(Nc4cc(C5CC5)[nH]n4)n3)C2)C1 ; CN1CC2(CCN(c3nccc(Nc4cc(C5CC5)[nH]n4)n3)CC2)C1 ; CNCC12CCC(C1)N(c1nccc(Nc3cc(C4CC4)[nH]n3)n1)C2] 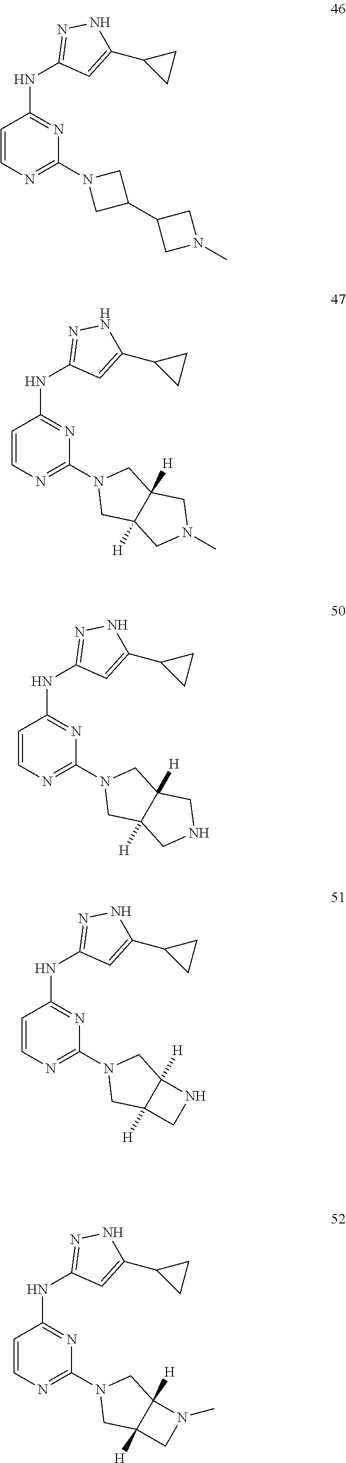 [SMILES: CN1CC(C2CN(c3nccc(Nc4cc(C5CC5)[nH]n4)n3)C2)C1 ; CN1C[C@H]2CN(c3nccc(Nc4cc(C5CC5)[nH]n4)n3)C[C@@H]2C1 ; c1cc(Nc2cc(C3CC3)[nH]n2)nc(N2C[C@@H]3CNC[C@H]3C2)n1 ; c1cc(Nc2cc(C3CC3)[nH]n2)nc(N2C[C@@H]3CN[C@@H]3C2)n1 ; CN1C[C@@H]2CN(c3nccc(Nc4cc(C5CC5)[nH]n4)n3)C[C@@H]21] 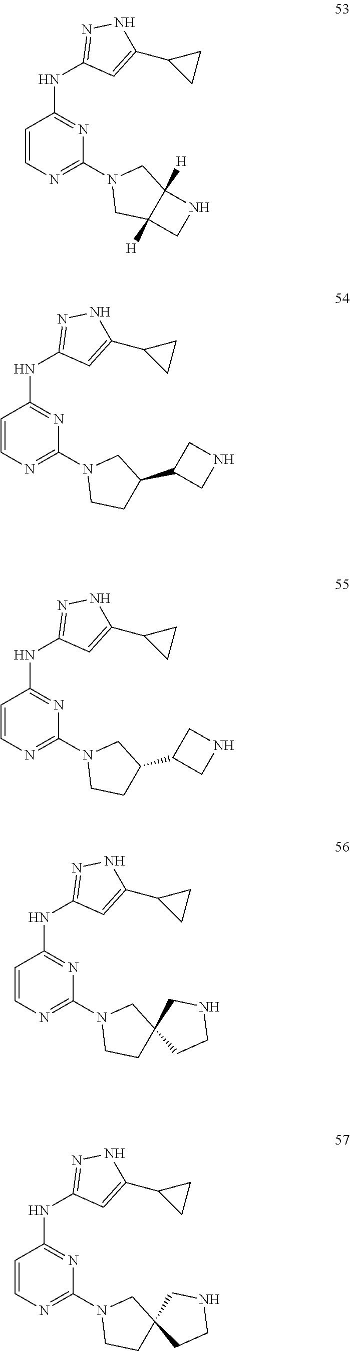 [SMILES: c1cc(Nc2cc(C3CC3)[nH]n2)nc(N2C[C@H]3CN[C@H]3C2)n1 ; c1cc(Nc2cc(C3CC3)[nH]n2)nc(N2CC[C@H](C3CNC3)C2)n1 ; c1cc(Nc2cc(C3CC3)[nH]n2)nc(N2CC[C@@H](C3CNC3)C2)n1 ; c1cc(Nc2cc(C3CC3)[nH]n2)nc(N2CC[C@@]3(CCNC3)C2)n1 ; c1cc(Nc2cc(C3CC3)[nH]n2)nc(N2CC[C@]3(CCNC3)C2)n1] 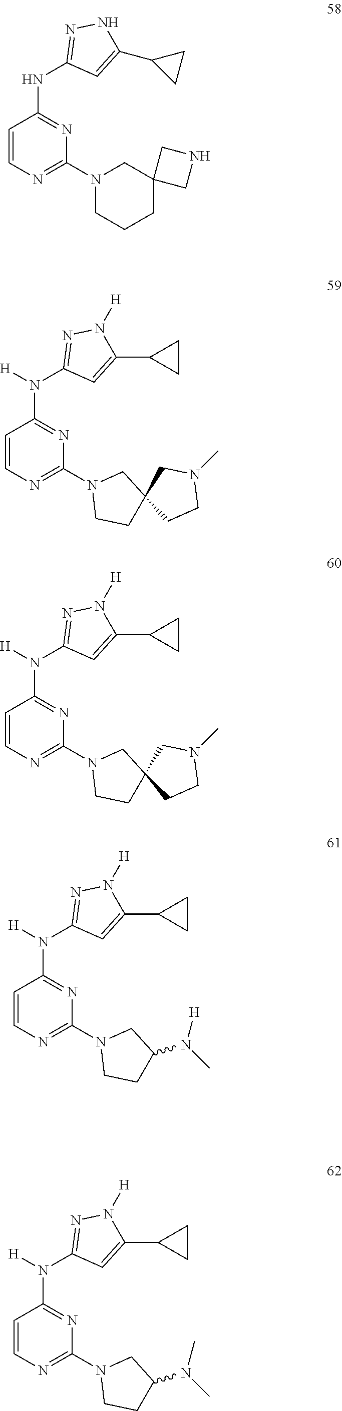 [SMILES: c1cc(Nc2cc(C3CC3)[nH]n2)nc(N2CCCC3(CNC3)C2)n1 ; CN1CC[C@@]2(CCN(c3nccc(Nc4cc(C5CC5)[nH]n4)n3)C2)C1 ; CN1CC[C@]2(CCN(c3nccc(Nc4cc(C5CC5)[nH]n4)n3)C2)C1 ; CNC1CCN(c2nccc(Nc3cc(C4CC4)[nH]n3)n2)C1 ; CN(C)C1CCN(c2nccc(Nc3cc(C4CC4)[nH]n3)n2)C1] 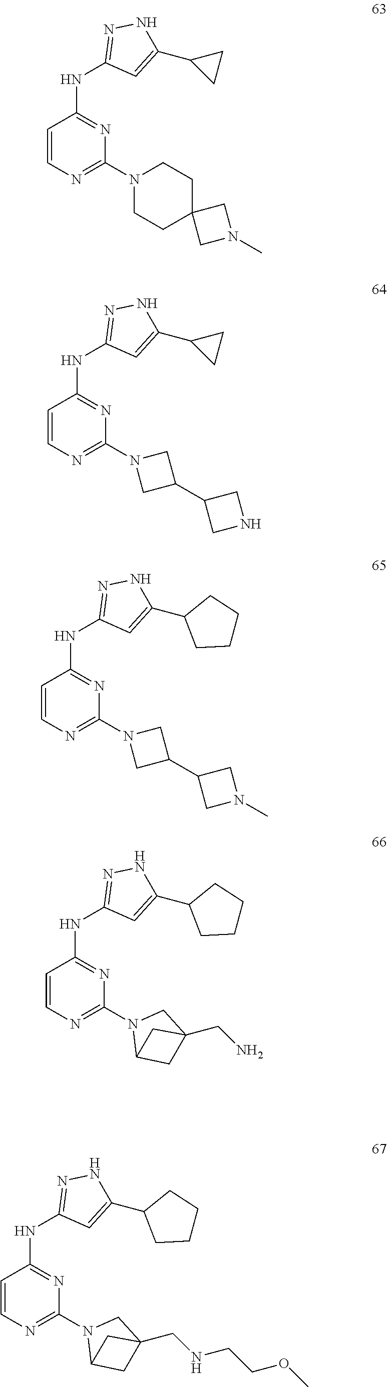 [SMILES: CN1CC2(CCN(c3nccc(Nc4cc(C5CC5)[nH]n4)n3)CC2)C1 ; c1cc(Nc2cc(C3CC3)[nH]n2)nc(N2CC(C3CNC3)C2)n1 ; CN1CC(C2CN(c3nccc(Nc4cc(C5CCCC5)[nH]n4)n3)C2)C1 ; NCC12CC(C1)N(c1nccc(Nc3cc(C4CCCC4)[nH]n3)n1)C2 ; COCCNCC12CC(C1)N(c1nccc(Nc3cc(C4CCCC4)[nH]n3)n1)C2] 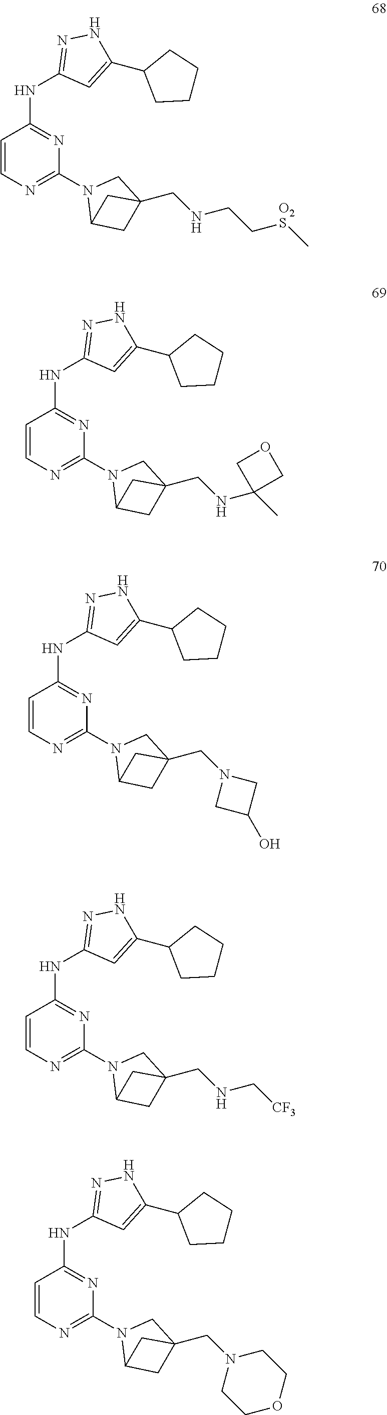 [SMILES: CS(=O)(=O)CCNCC12CC(C1)N(c1nccc(Nc3cc(C4CCCC4)[nH]n3)n1)C2 ; CC1(NCC23CC(C2)N(c2nccc(Nc4cc(C5CCCC5)[nH]n4)n2)C3)COC1 ; OC1CN(CC23CC(C2)N(c2nccc(Nc4cc(C5CCCC5)[nH]n4)n2)C3)C1 ; FC(F)(F)CNCC12CC(C1)N(c1nccc(Nc3cc(C4CCCC4)[nH]n3)n1)C2 ; c1cc(Nc2cc(C3CCCC3)[nH]n2)nc(N2CC3(CN4CCOCC4)CC2C3)n1] 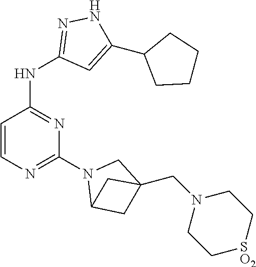 [SMILES: O=S1(=O)CCN(CC23CC(C2)N(c2nccc(Nc4cc(C5CCCC5)[nH]n4)n2)C3)CC1] 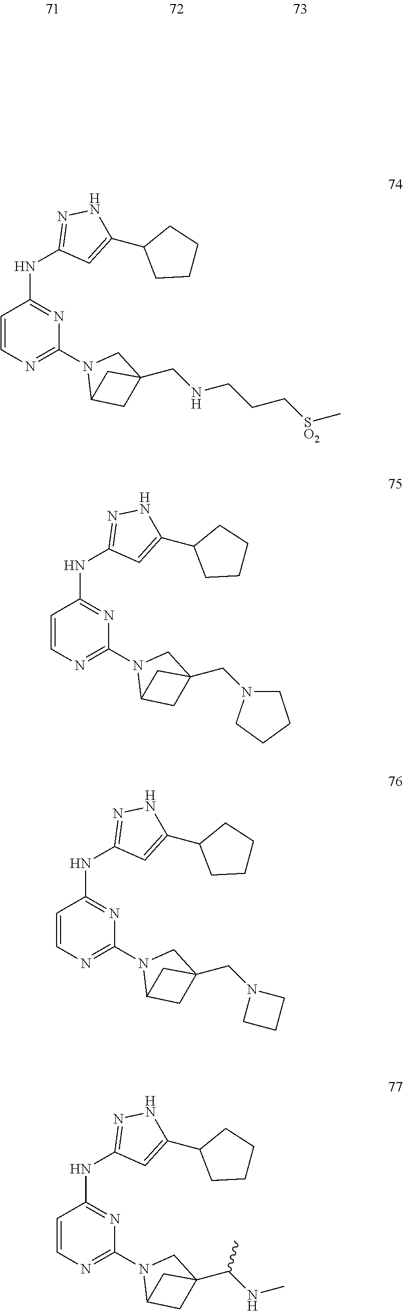 [SMILES: CS(=O)(=O)CCCNCC12CC(C1)N(c1nccc(Nc3cc(C4CCCC4)[nH]n3)n1)C2 ; c1cc(Nc2cc(C3CCCC3)[nH]n2)nc(N2CC3(CN4CCCC4)CC2C3)n1 ; c1cc(Nc2cc(C3CCCC3)[nH]n2)nc(N2CC3(CN4CCC4)CC2C3)n1 ; CNC(C)C12CC(C1)N(c1nccc(Nc3cc(C4CCCC4)[nH]n3)n1)C2] 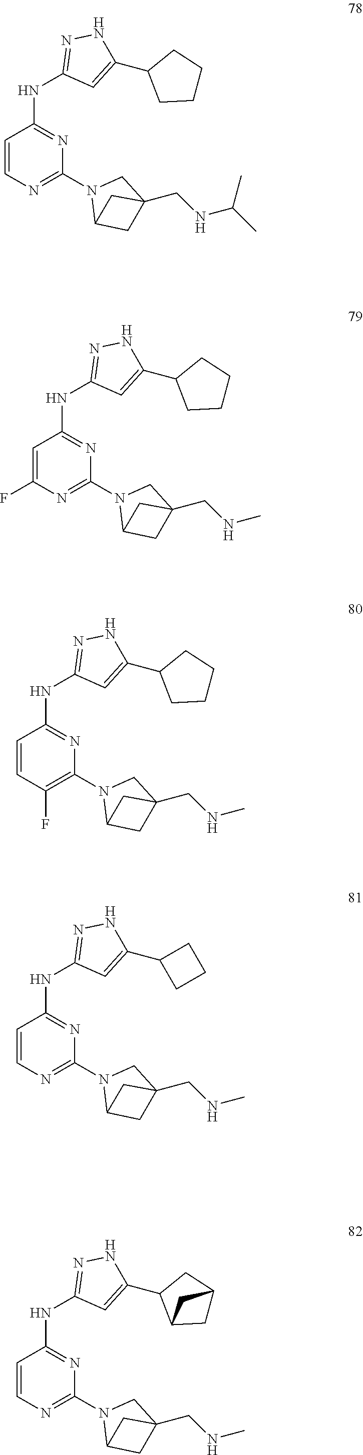 [SMILES: CC(C)NCC12CC(C1)N(c1nccc(Nc3cc(C4CCCC4)[nH]n3)n1)C2 ; CNCC12CC(C1)N(c1nc(F)cc(Nc3cc(C4CCCC4)[nH]n3)n1)C2 ; CNCC12CC(C1)N(c1nc(Nc3cc(C4CCCC4)[nH]n3)ccc1F)C2 ; CNCC12CC(C1)N(c1nccc(Nc3cc(C4CCC4)[nH]n3)n1)C2 ; CNCC12CC(C1)N(c1nccc(Nc3cc(C4C[C@H]5C[C@@H]4C5)[nH]n3)n1)C2] 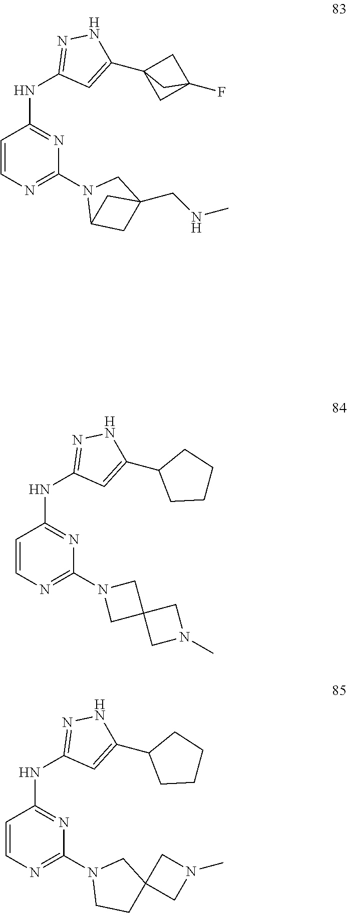 [SMILES: CNCC12CC(C1)N(c1nccc(Nc3cc(C45CC(F)(C4)C5)[nH]n3)n1)C2 ; CN1CC2(C1)CN(c1nccc(Nc3cc(C4CCCC4)[nH]n3)n1)C2 ; CN1CC2(CCN(c3nccc(Nc4cc(C5CCCC5)[nH]n4)n3)C2)C1] 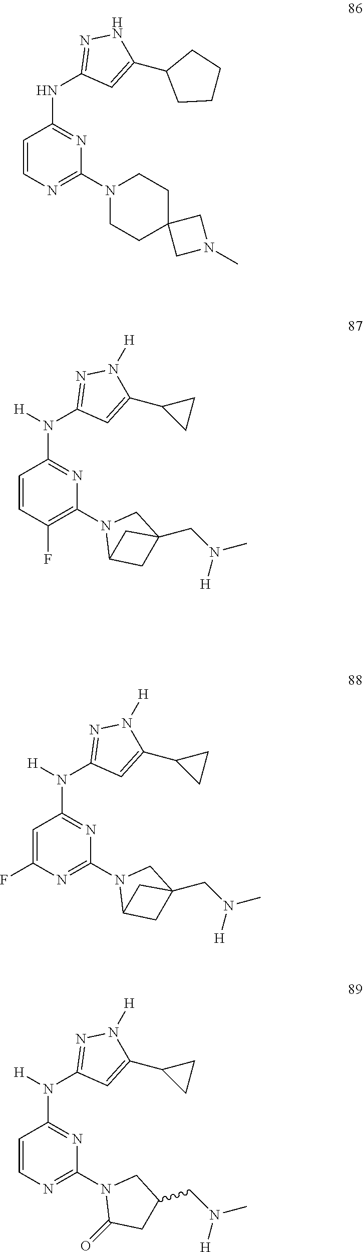 [SMILES: CN1CC2(CCN(c3nccc(Nc4cc(C5CCCC5)[nH]n4)n3)CC2)C1 ; CNCC12CC(C1)N(c1nc(Nc3cc(C4CC4)[nH]n3)ccc1F)C2 ; CNCC12CC(C1)N(c1nc(F)cc(Nc3cc(C4CC4)[nH]n3)n1)C2 ; CNCC1CC(=O)N(c2nccc(Nc3cc(C4CC4)[nH]n3)n2)C1] 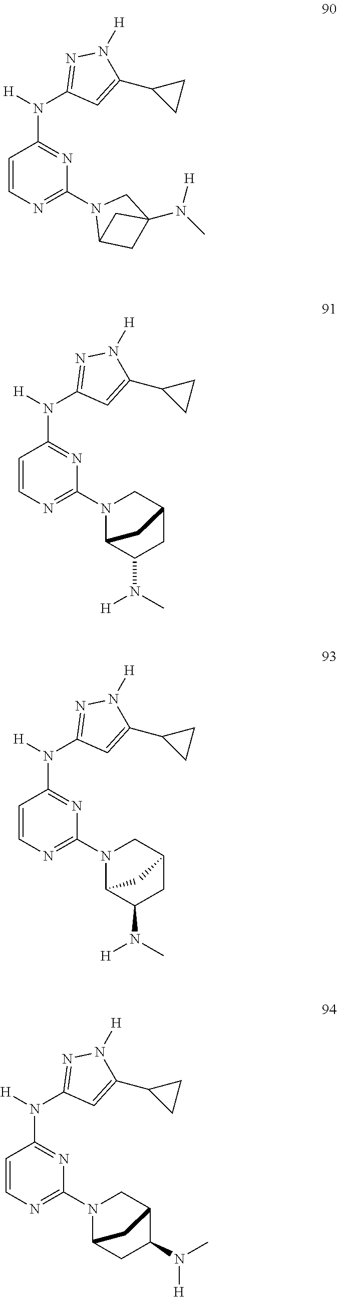 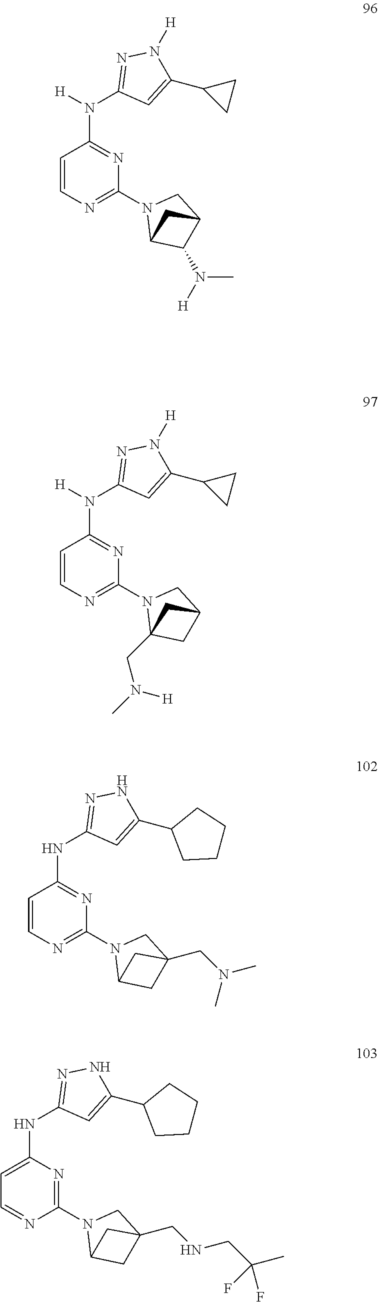 [SMILES: CN[C@H]1[C@@H]2C[C@H]1N(c1nccc(Nc3cc(C4CC4)[nH]n3)n1)C2 ; CNC[C@]12C[C@H](CN1c1nccc(Nc3cc(C4CC4)[nH]n3)n1)C2 ; CN(C)CC12CC(C1)N(c1nccc(Nc3cc(C4CCCC4)[nH]n3)n1)C2 ; CC(F)(F)CNCC12CC(C1)N(c1nccc(Nc3cc(C4CCCC4)[nH]n3)n1)C2] 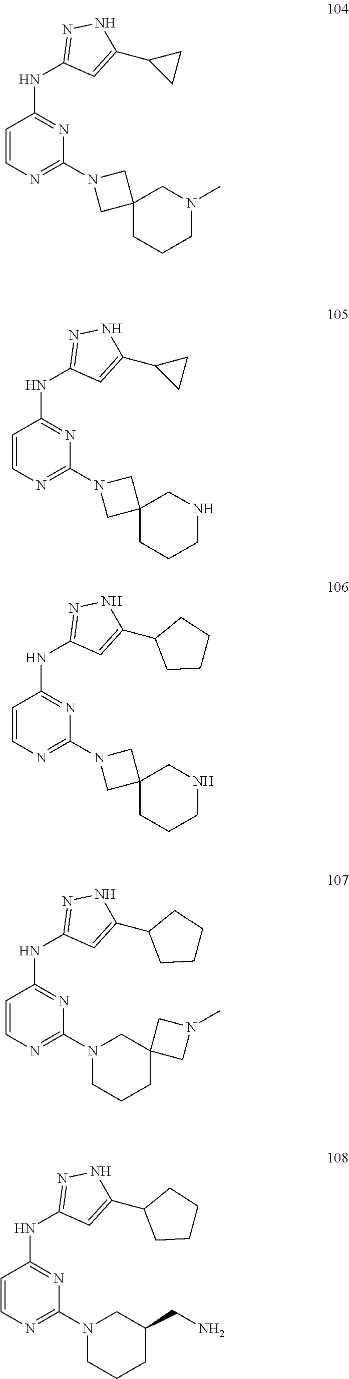 [SMILES: CN1CCCC2(C1)CN(c1nccc(Nc3cc(C4CC4)[nH]n3)n1)C2 ; c1cc(Nc2cc(C3CC3)[nH]n2)nc(N2CC3(CCCNC3)C2)n1 ; c1cc(Nc2cc(C3CCCC3)[nH]n2)nc(N2CC3(CCCNC3)C2)n1 ; CN1CC2(CCCN(c3nccc(Nc4cc(C5CCCC5)[nH]n4)n3)C2)C1 ; NC[C@H]1CCCN(c2nccc(Nc3cc(C4CCCC4)[nH]n3)n2)C1] 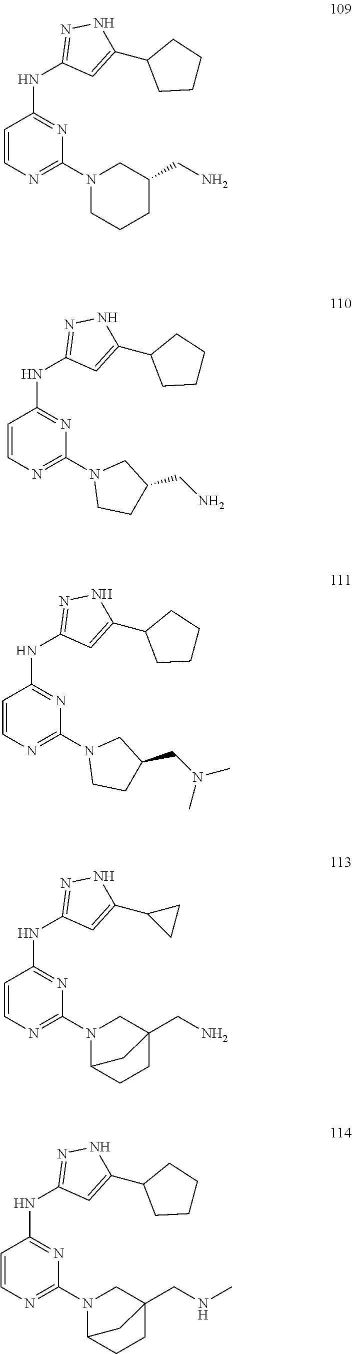 [SMILES: NC[C@@H]1CCCN(c2nccc(Nc3cc(C4CCCC4)[nH]n3)n2)C1 ; NC[C@@H]1CCN(c2nccc(Nc3cc(C4CCCC4)[nH]n3)n2)C1 ; CN(C)C[C@H]1CCN(c2nccc(Nc3cc(C4CCCC4)[nH]n3)n2)C1 ; NCC12CCC(C1)N(c1nccc(Nc3cc(C4CC4)[nH]n3)n1)C2 ; CNCC12CCC(C1)N(c1nccc(Nc3cc(C4CCCC4)[nH]n3)n1)C2] 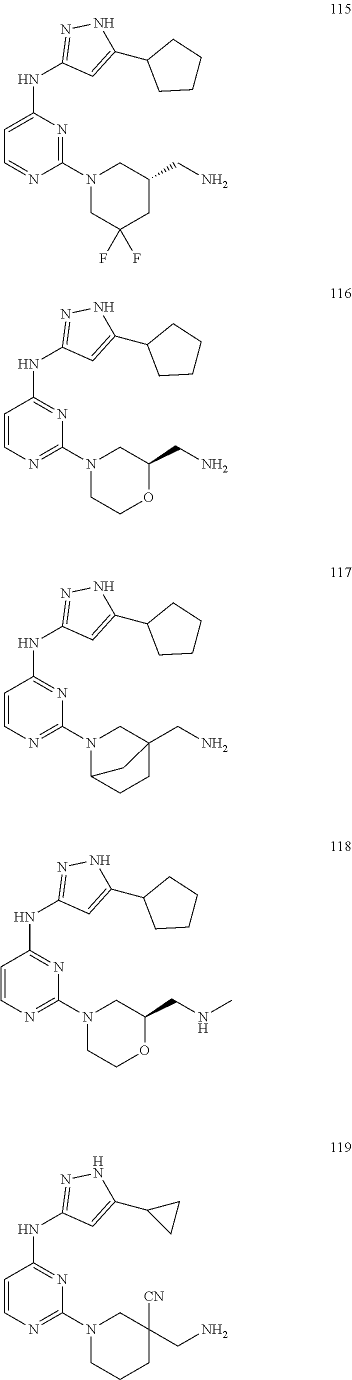 [SMILES: NC[C@H]1CN(c2nccc(Nc3cc(C4CCCC4)[nH]n3)n2)CC(F)(F)C1 ; NC[C@@H]1CN(c2nccc(Nc3cc(C4CCCC4)[nH]n3)n2)CCO1 ; NCC12CCC(C1)N(c1nccc(Nc3cc(C4CCCC4)[nH]n3)n1)C2 ; CNC[C@@H]1CN(c2nccc(Nc3cc(C4CCCC4)[nH]n3)n2)CCO1 ; N#CC1(CN)CCCN(c2nccc(Nc3cc(C4CC4)[nH]n3)n2)C1] 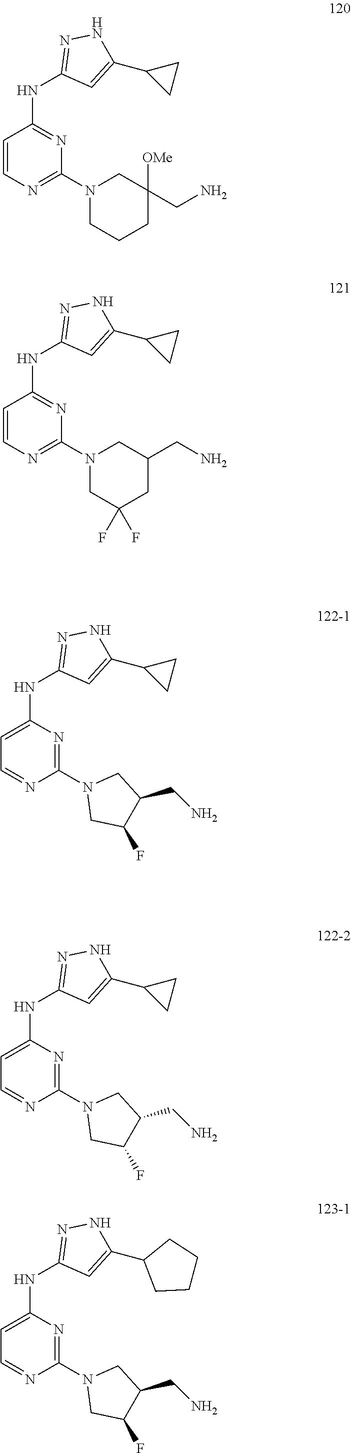 [SMILES: COC1(CN)CCCN(c2nccc(Nc3cc(C4CC4)[nH]n3)n2)C1 ; NCC1CN(c2nccc(Nc3cc(C4CC4)[nH]n3)n2)CC(F)(F)C1 ; NC[C@@H]1CN(c2nccc(Nc3cc(C4CC4)[nH]n3)n2)C[C@@H]1F ; NC[C@H]1CN(c2nccc(Nc3cc(C4CC4)[nH]n3)n2)C[C@H]1F ; NC[C@@H]1CN(c2nccc(Nc3cc(C4CCCC4)[nH]n3)n2)C[C@@H]1F] 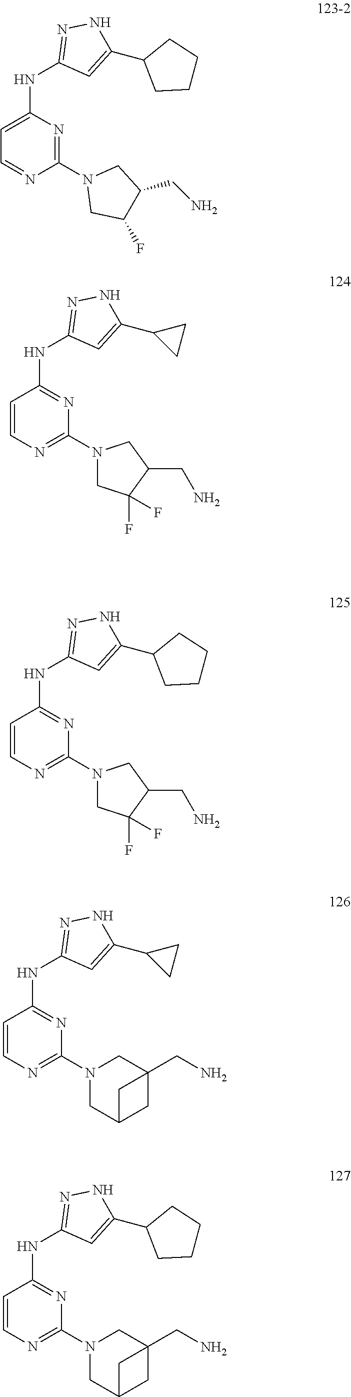 [SMILES: NC[C@H]1CN(c2nccc(Nc3cc(C4CCCC4)[nH]n3)n2)C[C@H]1F ; NCC1CN(c2nccc(Nc3cc(C4CC4)[nH]n3)n2)CC1(F)F ; NCC1CN(c2nccc(Nc3cc(C4CCCC4)[nH]n3)n2)CC1(F)F ; NCC12CC(CN(c3nccc(Nc4cc(C5CC5)[nH]n4)n3)C1)C2 ; NCC12CC(CN(c3nccc(Nc4cc(C5CCCC5)[nH]n4)n3)C1)C2] 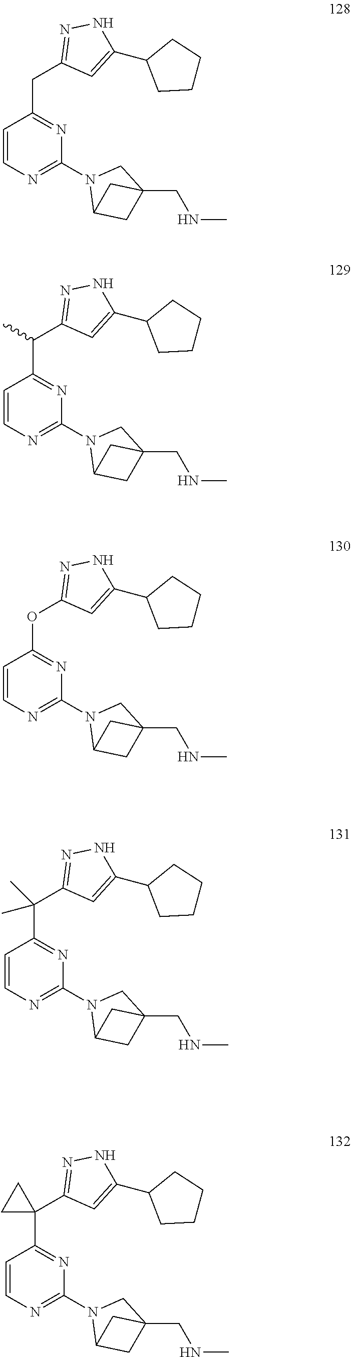 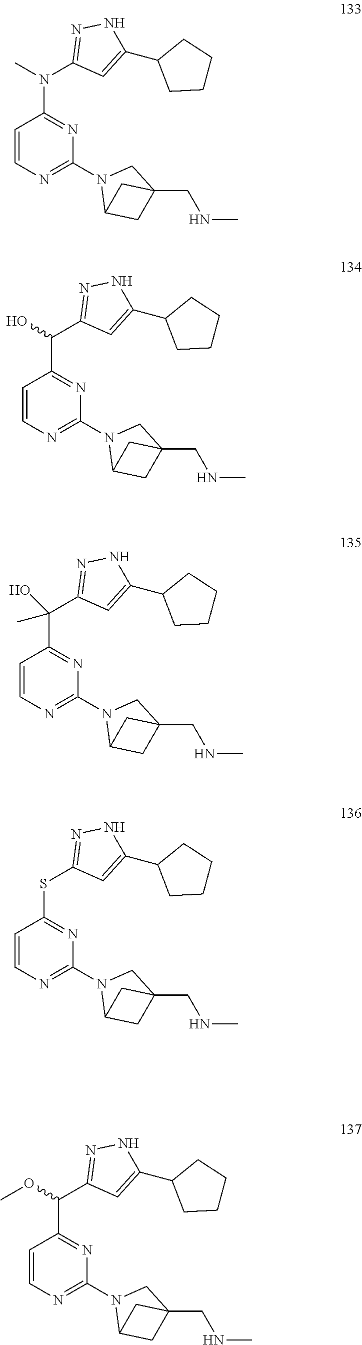 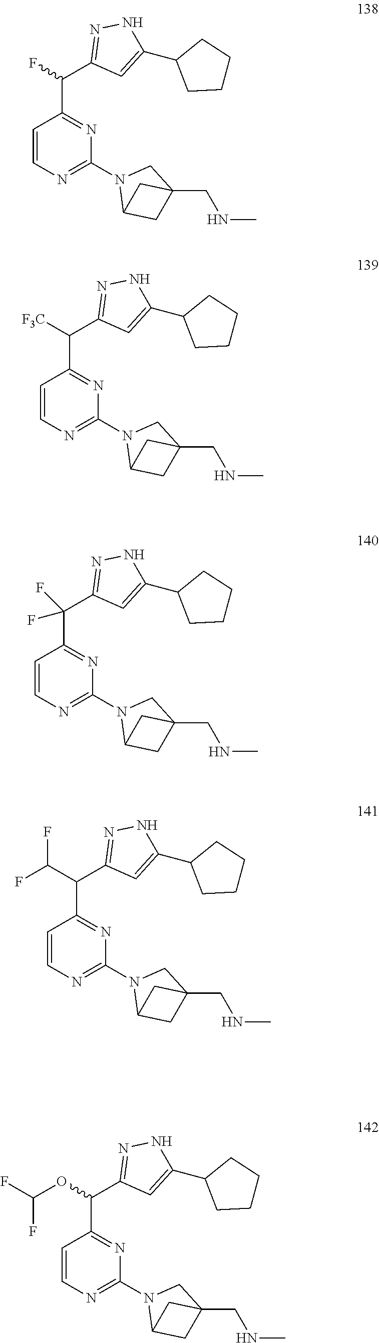 [SMILES: CNCC12CC(C1)N(c1nccc(C(F)c3cc(C4CCCC4)[nH]n3)n1)C2 ; CNCC12CC(C1)N(c1nccc(C(c3cc(C4CCCC4)[nH]n3)C(F)(F)F)n1)C2 ; CNCC12CC(C1)N(c1nccc(C(F)(F)c3cc(C4CCCC4)[nH]n3)n1)C2 ; CNCC12CC(C1)N(c1nccc(C(c3cc(C4CCCC4)[nH]n3)C(F)F)n1)C2 ; CNCC12CC(C1)N(c1nccc(C(OC(F)F)c3cc(C4CCCC4)[nH]n3)n1)C2] 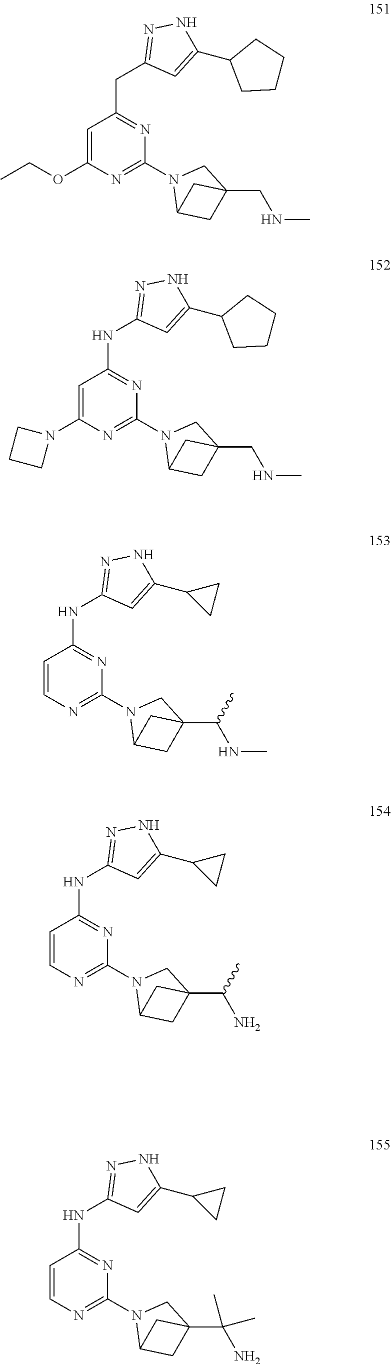 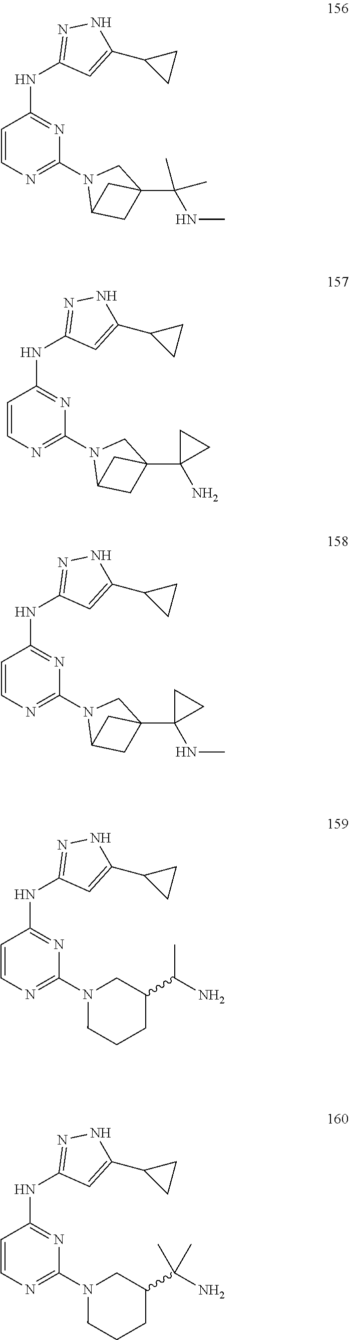 [SMILES: CNC(C)(C)C12CC(C1)N(c1nccc(Nc3cc(C4CC4)[nH]n3)n1)C2 ; NC1(C23CC(C2)N(c2nccc(Nc4cc(C5CC5)[nH]n4)n2)C3)CC1 ; CNC1(C23CC(C2)N(c2nccc(Nc4cc(C5CC5)[nH]n4)n2)C3)CC1 ; CC(N)C1CCCN(c2nccc(Nc3cc(C4CC4)[nH]n3)n2)C1 ; CC(C)(N)C1CCCN(c2nccc(Nc3cc(C4CC4)[nH]n3)n2)C1] 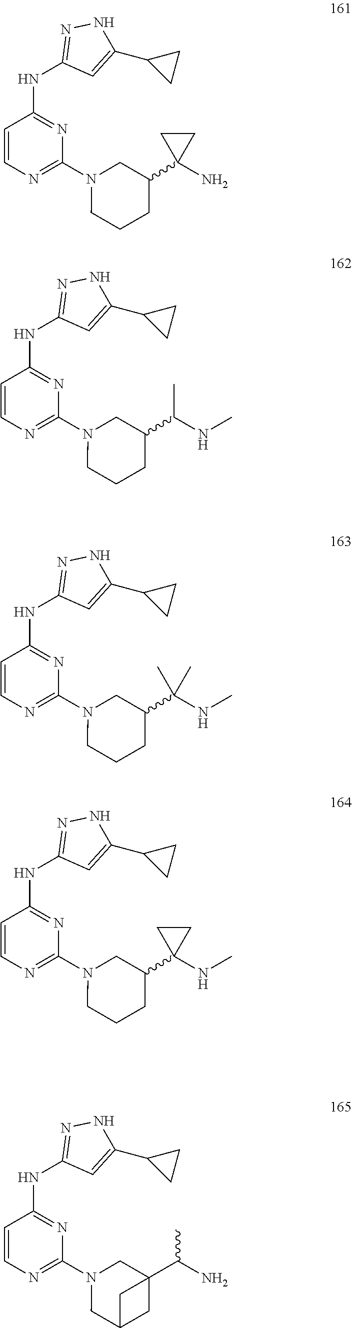 [SMILES: NC1(C2CCCN(c3nccc(Nc4cc(C5CC5)[nH]n4)n3)C2)CC1 ; CNC(C)C1CCCN(c2nccc(Nc3cc(C4CC4)[nH]n3)n2)C1 ; CNC(C)(C)C1CCCN(c2nccc(Nc3cc(C4CC4)[nH]n3)n2)C1 ; CNC1(C2CCCN(c3nccc(Nc4cc(C5CC5)[nH]n4)n3)C2)CC1 ; CC(N)C12CC(CN(c3nccc(Nc4cc(C5CC5)[nH]n4)n3)C1)C2] 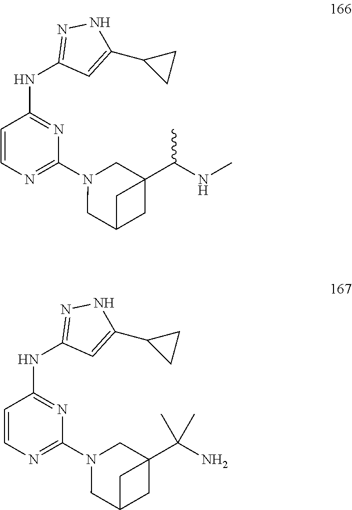 [SMILES: CNC(C)C12CC(CN(c3nccc(Nc4cc(C5CC5)[nH]n4)n3)C1)C2 ; CC(C)(N)C12CC(CN(c3nccc(Nc4cc(C5CC5)[nH]n4)n3)C1)C2] 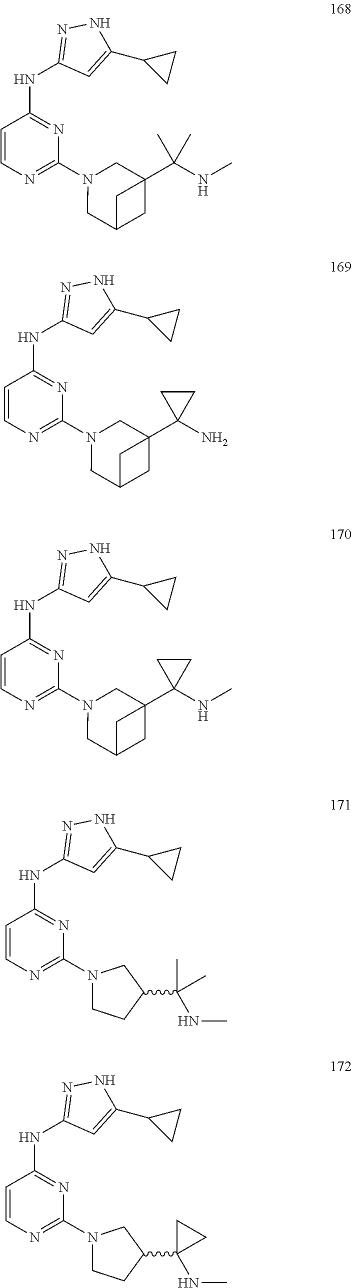 [SMILES: CNC(C)(C)C12CC(CN(c3nccc(Nc4cc(C5CC5)[nH]n4)n3)C1)C2 ; NC1(C23CC(CN(c4nccc(Nc5cc(C6CC6)[nH]n5)n4)C2)C3)CC1 ; CNC1(C23CC(CN(c4nccc(Nc5cc(C6CC6)[nH]n5)n4)C2)C3)CC1 ; CNC(C)(C)C1CCN(c2nccc(Nc3cc(C4CC4)[nH]n3)n2)C1 ; CNC1(C2CCN(c3nccc(Nc4cc(C5CC5)[nH]n4)n3)C2)CC1] 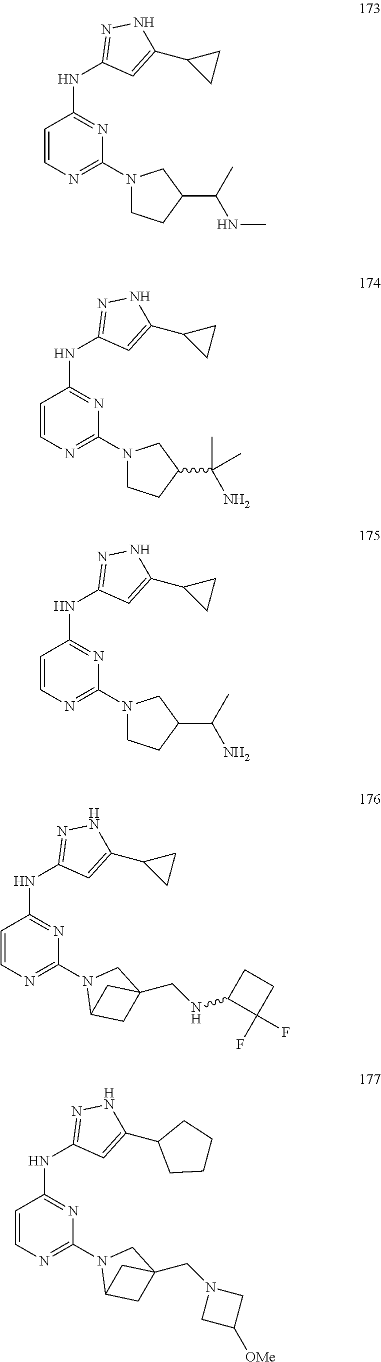 [SMILES: CNC(C)C1CCN(c2nccc(Nc3cc(C4CC4)[nH]n3)n2)C1 ; CC(C)(N)C1CCN(c2nccc(Nc3cc(C4CC4)[nH]n3)n2)C1 ; CC(N)C1CCN(c2nccc(Nc3cc(C4CC4)[nH]n3)n2)C1 ; FC1(F)CCC1NCC12CC(C1)N(c1nccc(Nc3cc(C4CC4)[nH]n3)n1)C2 ; COC1CN(CC23CC(C2)N(c2nccc(Nc4cc(C5CCCC5)[nH]n4)n2)C3)C1] 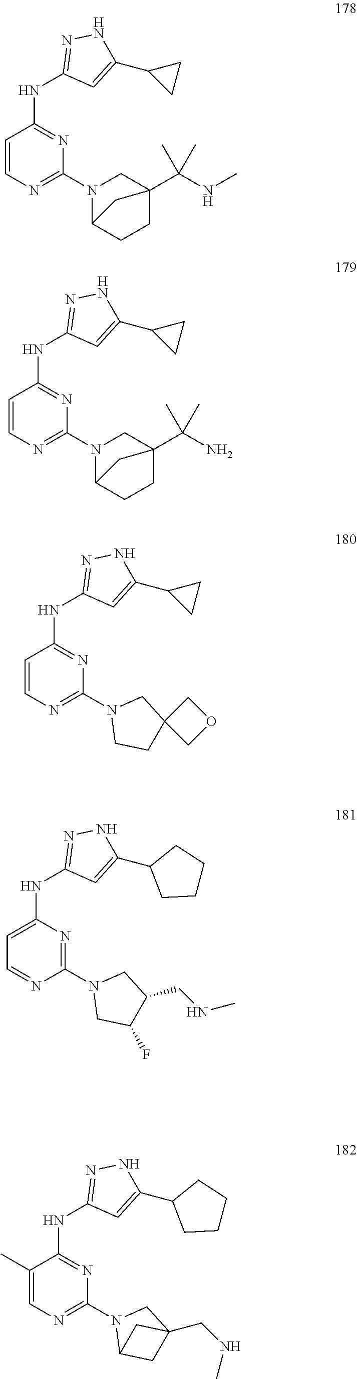 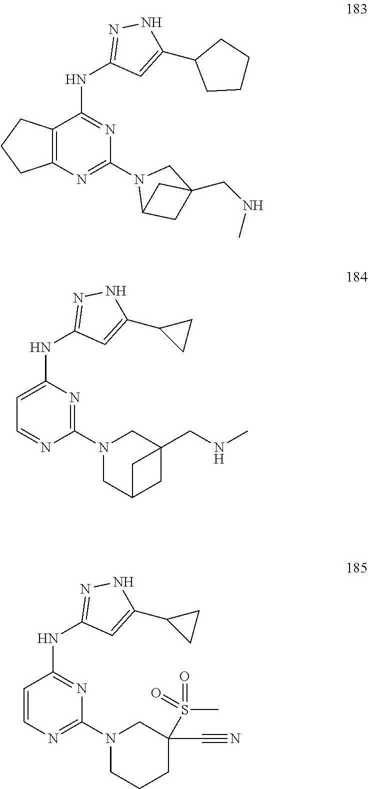 [SMILES: CNCC12CC(C1)N(c1nc3c(c(Nc4cc(C5CCCC5)[nH]n4)n1)CCC3)C2 ; CNCC12CC(CN(c3nccc(Nc4cc(C5CC5)[nH]n4)n3)C1)C2 ; CS(=O)(=O)C1(C#N)CCCN(c2nccc(Nc3cc(C4CC4)[nH]n3)n2)C1] 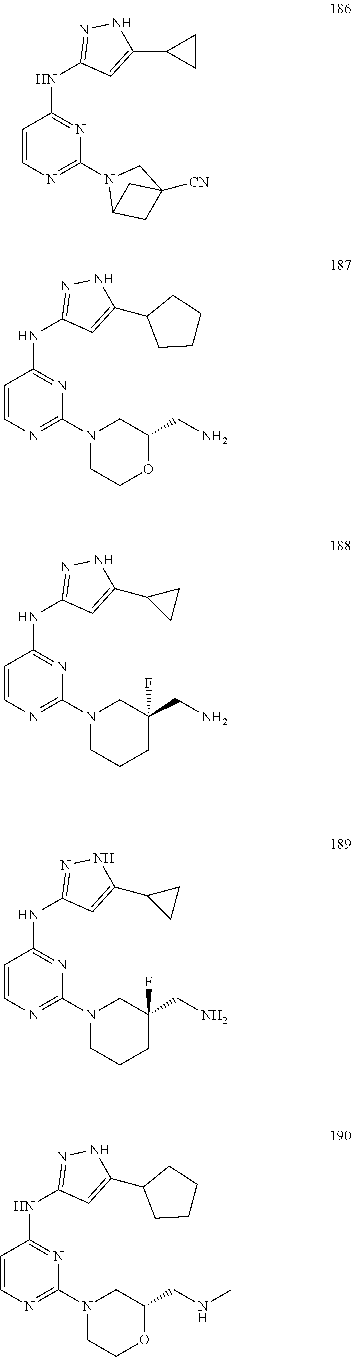 [SMILES: N#CC12CC(C1)N(c1nccc(Nc3cc(C4CC4)[nH]n3)n1)C2 ; NC[C@H]1CN(c2nccc(Nc3cc(C4CCCC4)[nH]n3)n2)CCO1 ; NC[C@@]1(F)CCCN(c2nccc(Nc3cc(C4CC4)[nH]n3)n2)C1 ; NC[C@]1(F)CCCN(c2nccc(Nc3cc(C4CC4)[nH]n3)n2)C1 ; CNC[C@H]1CN(c2nccc(Nc3cc(C4CCCC4)[nH]n3)n2)CCO1] 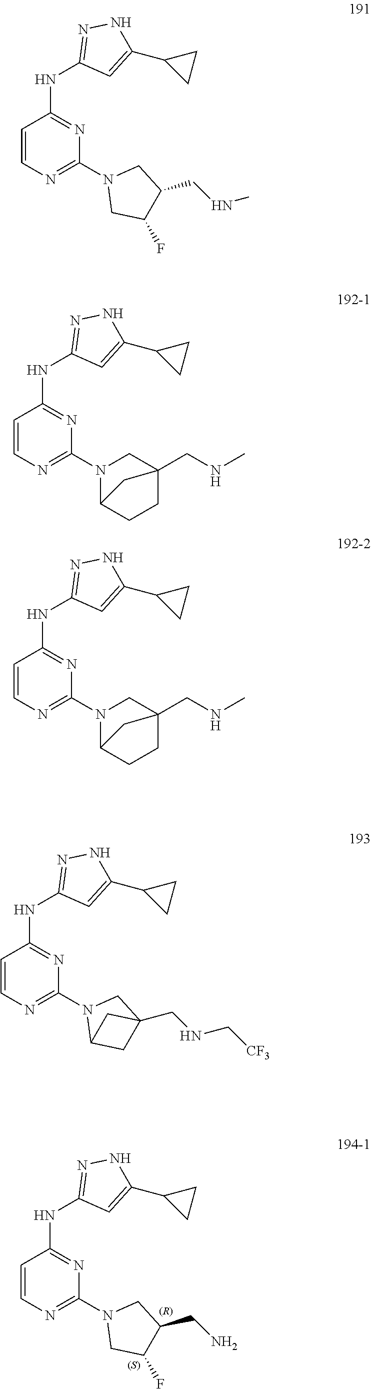 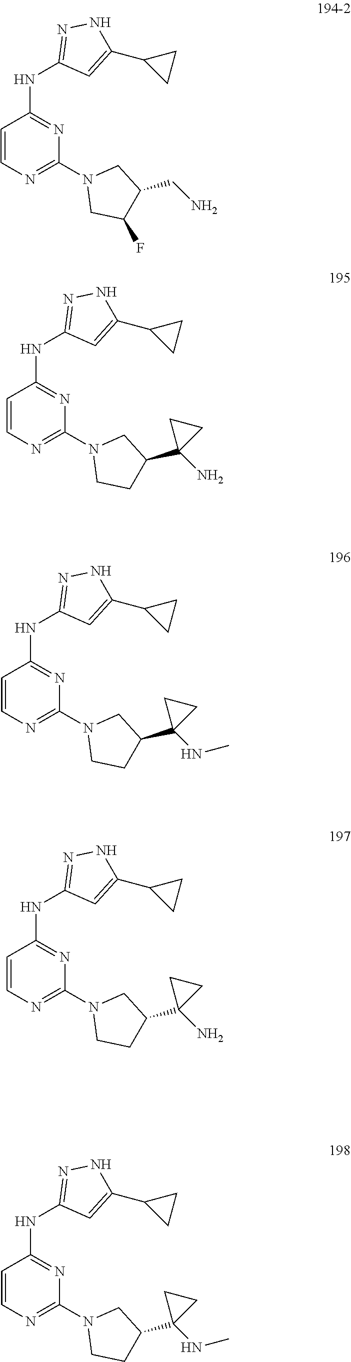 [SMILES: NC[C@H]1CN(c2nccc(Nc3cc(C4CC4)[nH]n3)n2)C[C@@H]1F ; NC1([C@H]2CCN(c3nccc(Nc4cc(C5CC5)[nH]n4)n3)C2)CC1 ; CNC1([C@H]2CCN(c3nccc(Nc4cc(C5CC5)[nH]n4)n3)C2)CC1 ; NC1([C@@H]2CCN(c3nccc(Nc4cc(C5CC5)[nH]n4)n3)C2)CC1 ; CNC1([C@@H]2CCN(c3nccc(Nc4cc(C5CC5)[nH]n4)n3)C2)CC1] 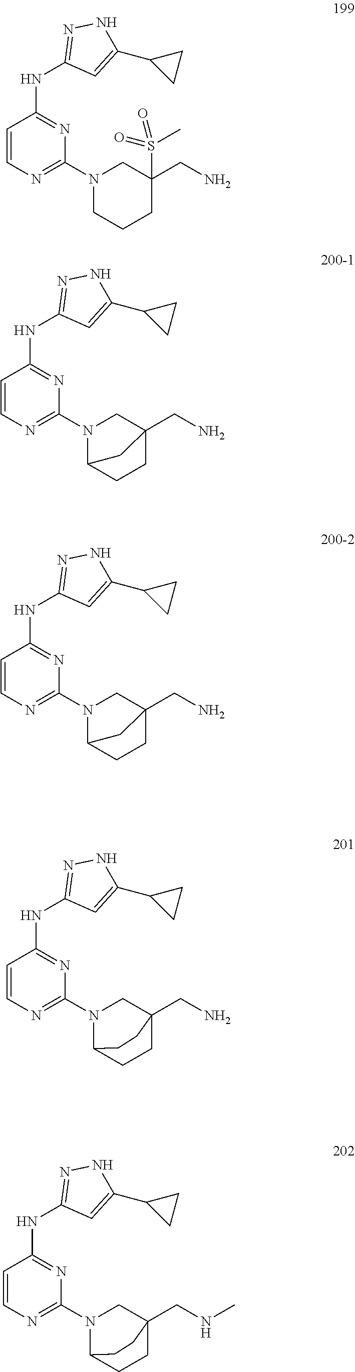 [SMILES: CS(=O)(=O)C1(CN)CCCN(c2nccc(Nc3cc(C4CC4)[nH]n3)n2)C1 ; NCC12CCC(C1)N(c1nccc(Nc3cc(C4CC4)[nH]n3)n1)C2 ; NCC12CCC(C1)N(c1nccc(Nc3cc(C4CC4)[nH]n3)n1)C2 ; NCC12CCC(CC1)N(c1nccc(Nc3cc(C4CC4)[nH]n3)n1)C2 ; CNCC12CCC(CC1)N(c1nccc(Nc3cc(C4CC4)[nH]n3)n1)C2] 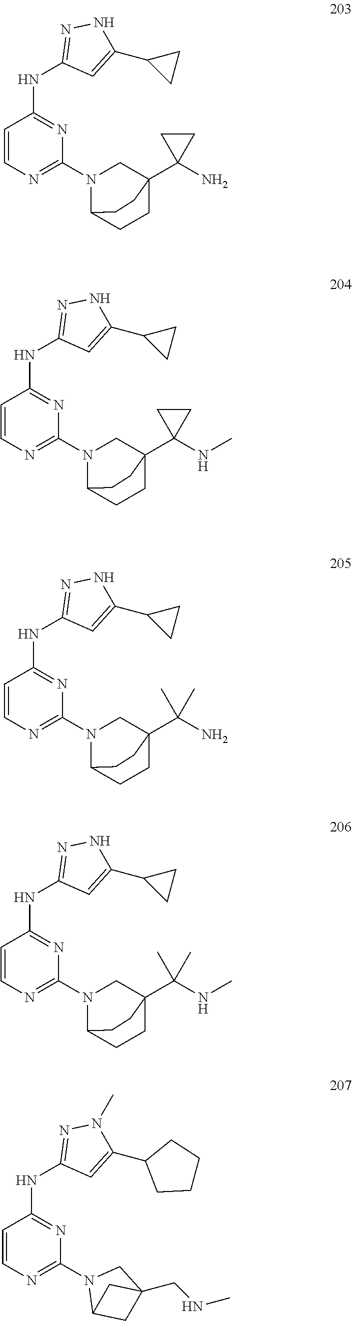 [SMILES: NC1(C23CCC(CC2)N(c2nccc(Nc4cc(C5CC5)[nH]n4)n2)C3)CC1 ; CNC1(C23CCC(CC2)N(c2nccc(Nc4cc(C5CC5)[nH]n4)n2)C3)CC1 ; CC(C)(N)C12CCC(CC1)N(c1nccc(Nc3cc(C4CC4)[nH]n3)n1)C2 ; CNC(C)(C)C12CCC(CC1)N(c1nccc(Nc3cc(C4CC4)[nH]n3)n1)C2 ; CNCC12CC(C1)N(c1nccc(Nc3cc(C4CCCC4)n(C)n3)n1)C2] 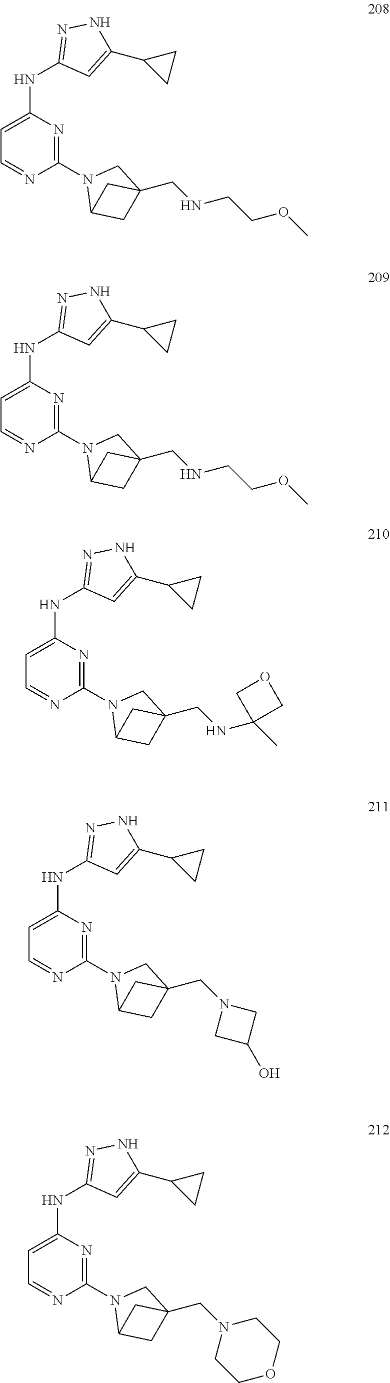 [SMILES: COCCNCC12CC(C1)N(c1nccc(Nc3cc(C4CC4)[nH]n3)n1)C2 ; COCCNCC12CC(C1)N(c1nccc(Nc3cc(C4CC4)[nH]n3)n1)C2 ; CC1(NCC23CC(C2)N(c2nccc(Nc4cc(C5CC5)[nH]n4)n2)C3)COC1 ; OC1CN(CC23CC(C2)N(c2nccc(Nc4cc(C5CC5)[nH]n4)n2)C3)C1 ; c1cc(Nc2cc(C3CC3)[nH]n2)nc(N2CC3(CN4CCOCC4)CC2C3)n1] 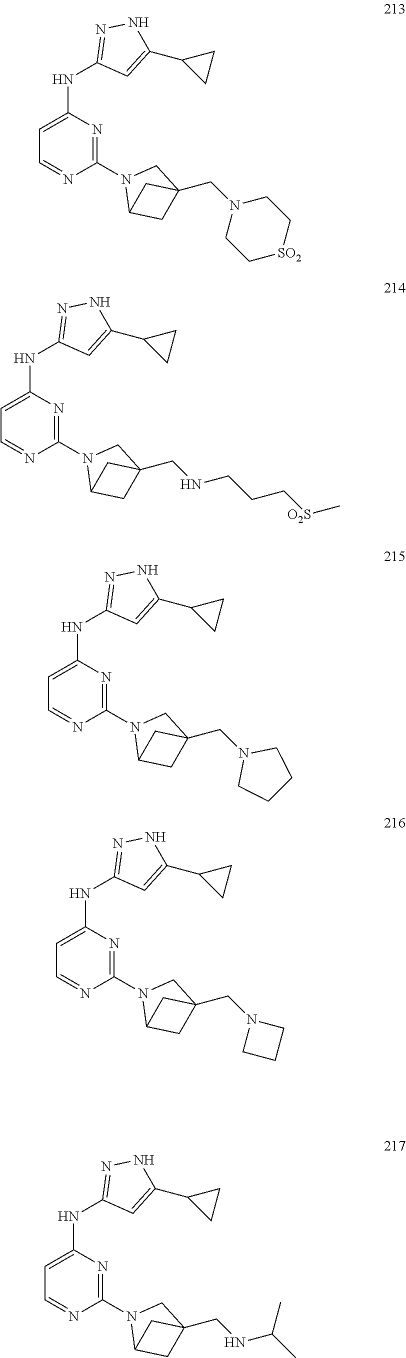 [SMILES: O=S1(=O)CCN(CC23CC(C2)N(c2nccc(Nc4cc(C5CC5)[nH]n4)n2)C3)CC1 ; CS(=O)(=O)CCCNCC12CC(C1)N(c1nccc(Nc3cc(C4CC4)[nH]n3)n1)C2 ; c1cc(Nc2cc(C3CC3)[nH]n2)nc(N2CC3(CN4CCCC4)CC2C3)n1 ; c1cc(Nc2cc(C3CC3)[nH]n2)nc(N2CC3(CN4CCC4)CC2C3)n1 ; CC(C)NCC12CC(C1)N(c1nccc(Nc3cc(C4CC4)[nH]n3)n1)C2] 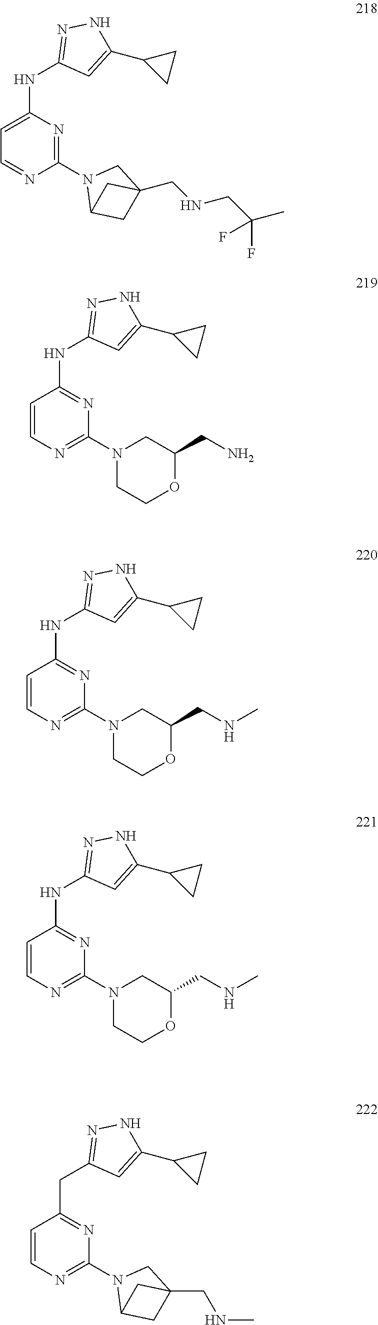 [SMILES: CC(F)(F)CNCC12CC(C1)N(c1nccc(Nc3cc(C4CC4)[nH]n3)n1)C2 ; NC[C@@H]1CN(c2nccc(Nc3cc(C4CC4)[nH]n3)n2)CCO1 ; CNC[C@@H]1CN(c2nccc(Nc3cc(C4CC4)[nH]n3)n2)CCO1 ; CNC[C@H]1CN(c2nccc(Nc3cc(C4CC4)[nH]n3)n2)CCO1 ; CNCC12CC(C1)N(c1nccc(Cc3cc(C4CC4)[nH]n3)n1)C2] 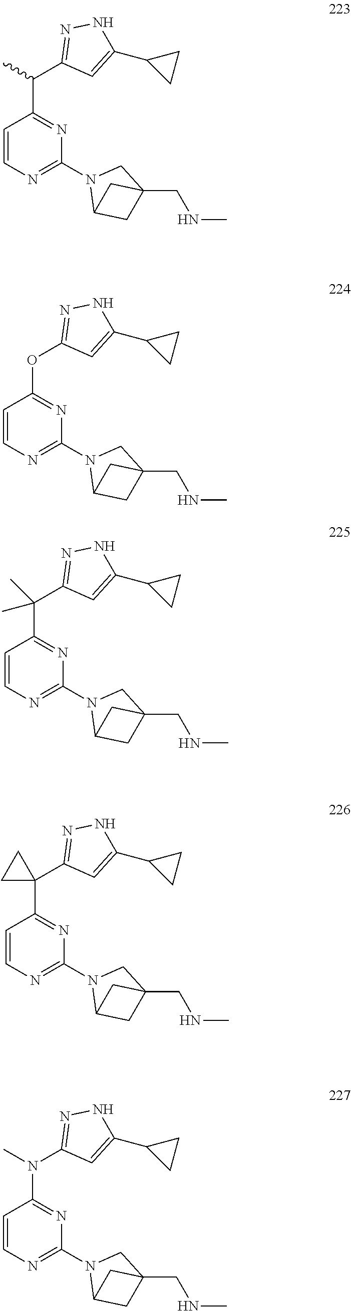 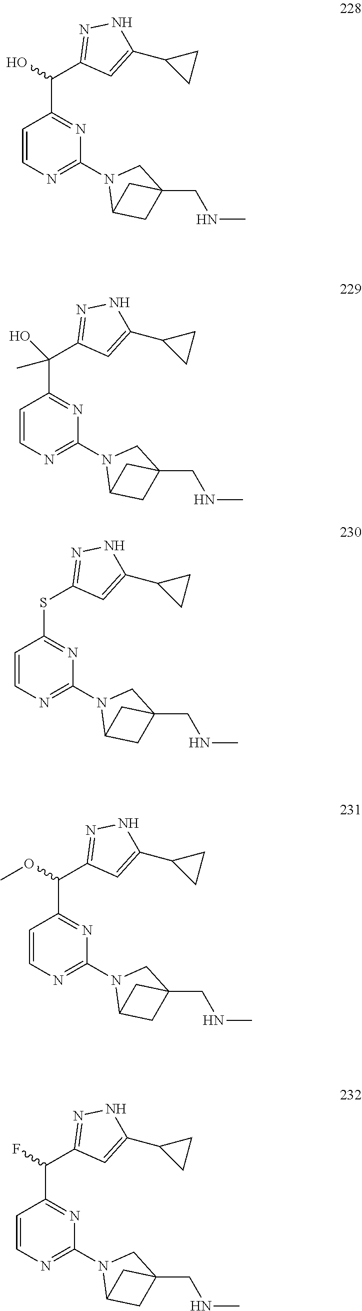 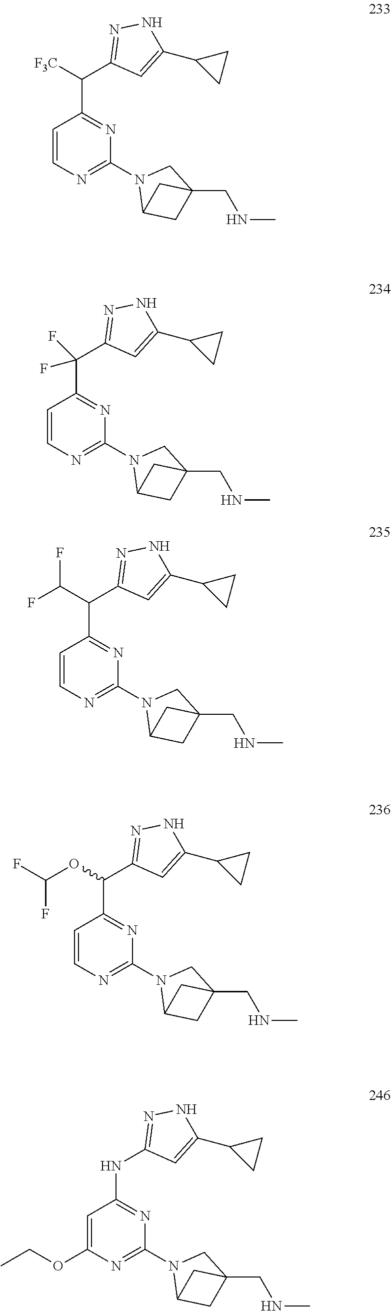 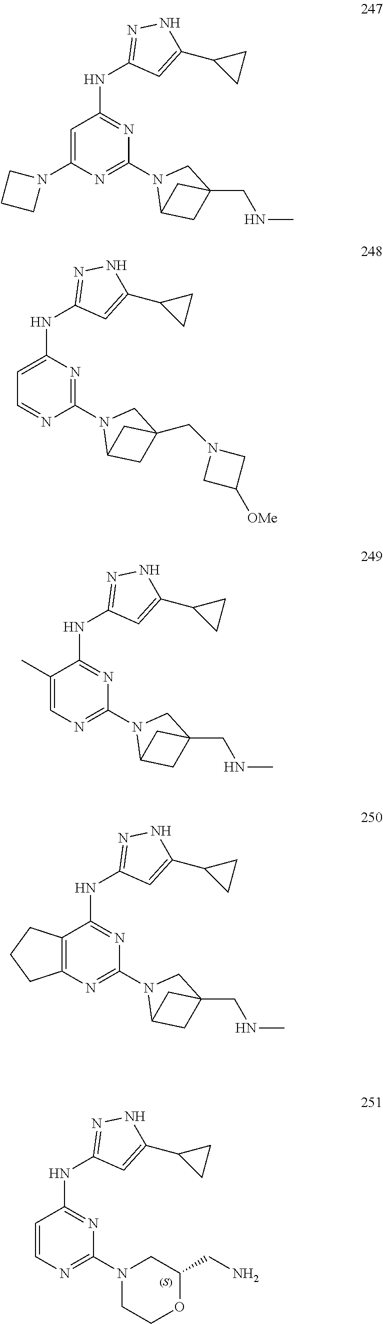 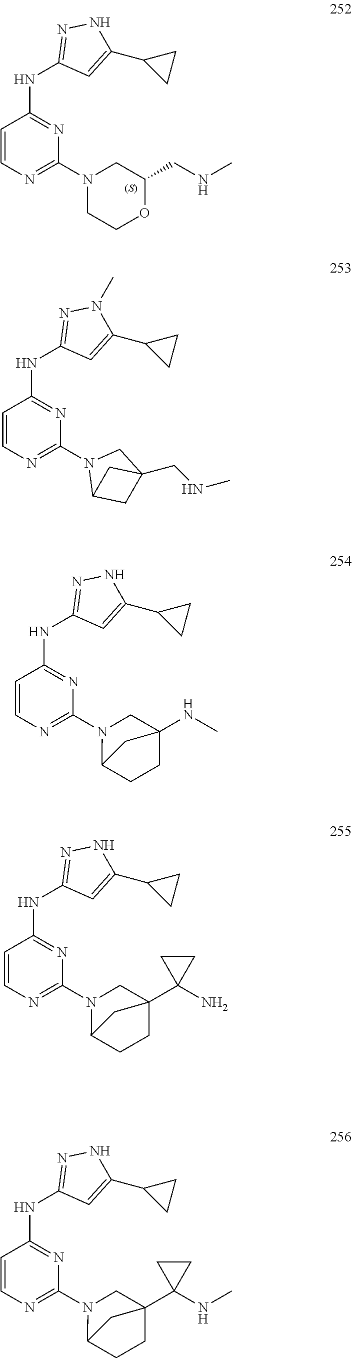 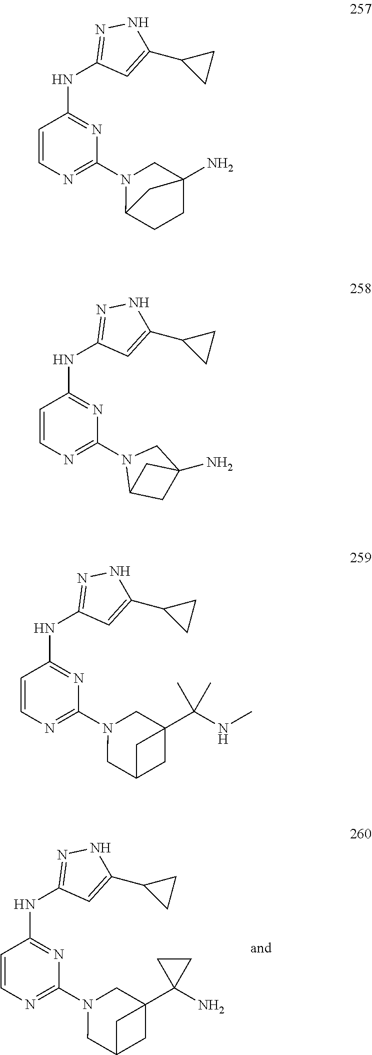 [SMILES: NC12CCC(C1)N(c1nccc(Nc3cc(C4CC4)[nH]n3)n1)C2 ; NC12CC(C1)N(c1nccc(Nc3cc(C4CC4)[nH]n3)n1)C2 ; CNC(C)(C)C12CC(CN(c3nccc(Nc4cc(C5CC5)[nH]n4)n3)C1)C2 ; NC1(C23CC(CN(c4nccc(Nc5cc(C6CC6)[nH]n5)n4)C2)C3)CC1] 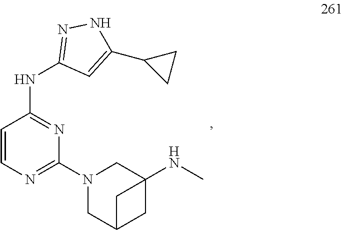 [SMILES: CNC12CC(CN(c3nccc(Nc4cc(C5CC5)[nH]n4)n3)C1)C2]
or a pharmaceutically acceptable salt, stereoisomer, and/or N-oxide thereof.

31. A compound of Formula II: 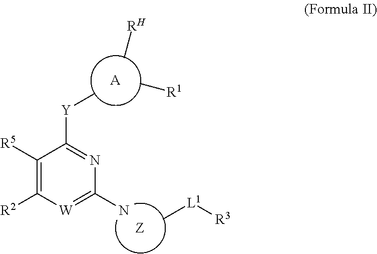
or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
ring A is a 5-6 membered heteroaryl optionally substituted with C₁-C₄ alkyl;
ring Z is selected from the group consisting of 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, oxo, C₁-C₄alkoxy, and cyano;
L¹ is bond or C₁-C₆ alkylene (optionally substituted by one, two or three R⁴);
R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
R⁴ is each independently selected from the group consisting of halogen and C₁-C₆ alkyl, or two R⁴ together with the carbon from which the R⁴ are attached form a C₃-C₅ carbocyclyl;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R^{N} is selected from the group consisting of H and C₁-C₃alkyl, or two R^{N} together with the nitrogen atoms to which the R^{N}s are attached form a 4-6 membered heterocyclic ring;
R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
R² is selected from the group consisting of H, F, CF₃, —C(O)—O-methyl, —C(O)OH, —O-methyl, —O-ethyl, C₃-C₇ cycloalkyl, and heterocyclyl; and
R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring.
A compound selected from the group consisting of: 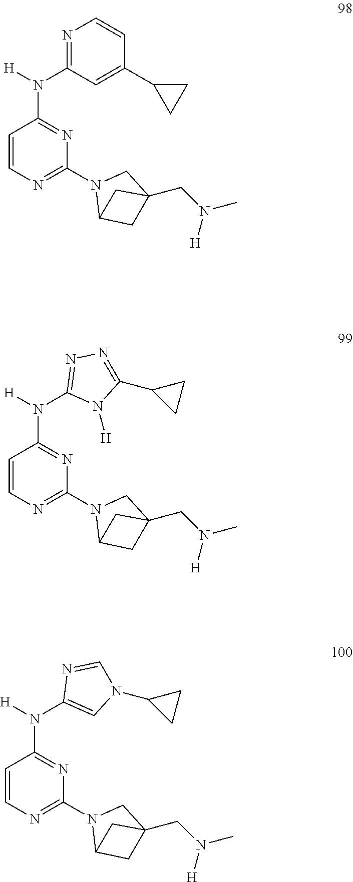 [SMILES: CNCC12CC(C1)N(c1nccc(Nc3cc(C4CC4)ccn3)n1)C2 ; CNCC12CC(C1)N(c1nccc(Nc3nnc(C4CC4)[nH]3)n1)C2 ; CNCC12CC(C1)N(c1nccc(Nc3cn(C4CC4)cn3)n1)C2] 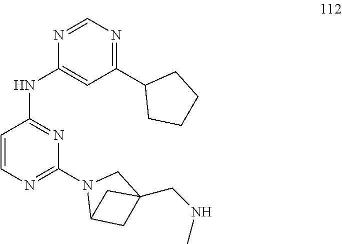 [SMILES: CNCC12CC(C1)N(c1nccc(Nc3cc(C4CCCC4)ncn3)n1)C2] 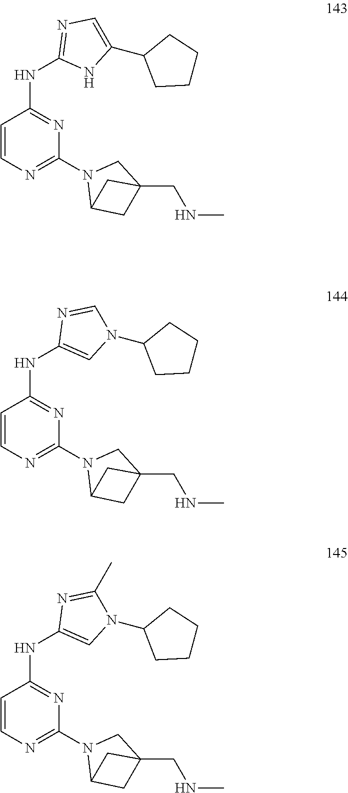 [SMILES: CNCC12CC(C1)N(c1nccc(Nc3ncc(C4CCCC4)[nH]3)n1)C2 ; CNCC12CC(C1)N(c1nccc(Nc3cn(C4CCCC4)cn3)n1)C2 ; CNCC12CC(C1)N(c1nccc(Nc3cn(C4CCCC4)c(C)n3)n1)C2] 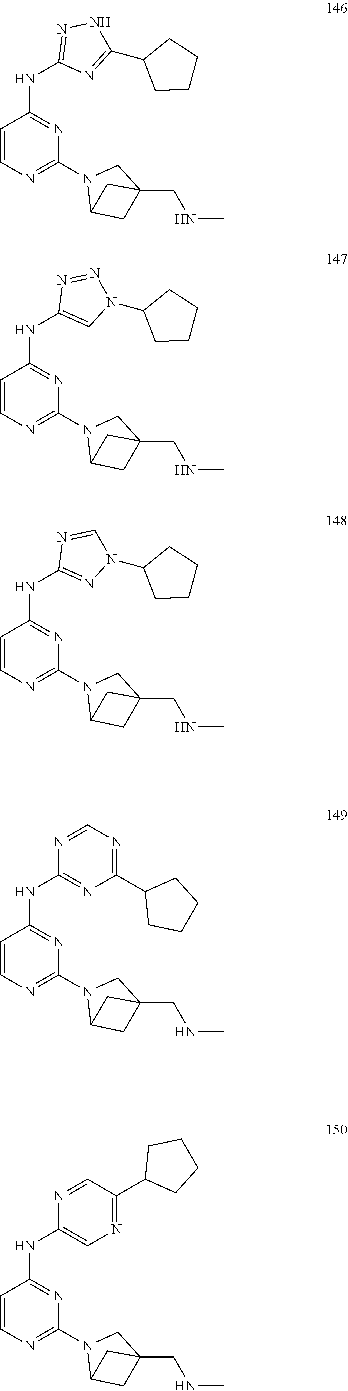 [SMILES: CNCC12CC(C1)N(c1nccc(Nc3n[nH]c(C4CCCC4)n3)n1)C2 ; CNCC12CC(C1)N(c1nccc(Nc3cn(C4CCCC4)nn3)n1)C2 ; CNCC12CC(C1)N(c1nccc(Nc3ncn(C4CCCC4)n3)n1)C2 ; CNCC12CC(C1)N(c1nccc(Nc3ncnc(C4CCCC4)n3)n1)C2 ; CNCC12CC(C1)N(c1nccc(Nc3cnc(C4CCCC4)cn3)n1)C2] 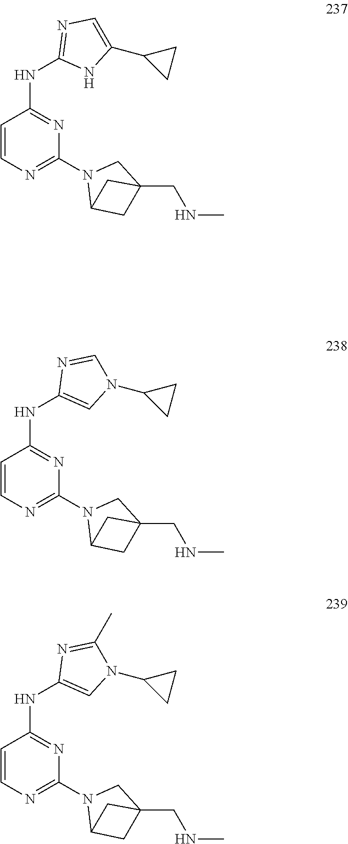 [SMILES: CNCC12CC(C1)N(c1nccc(Nc3ncc(C4CC4)[nH]3)n1)C2 ; CNCC12CC(C1)N(c1nccc(Nc3cn(C4CC4)cn3)n1)C2 ; CNCC12CC(C1)N(c1nccc(Nc3cn(C4CC4)c(C)n3)n1)C2] 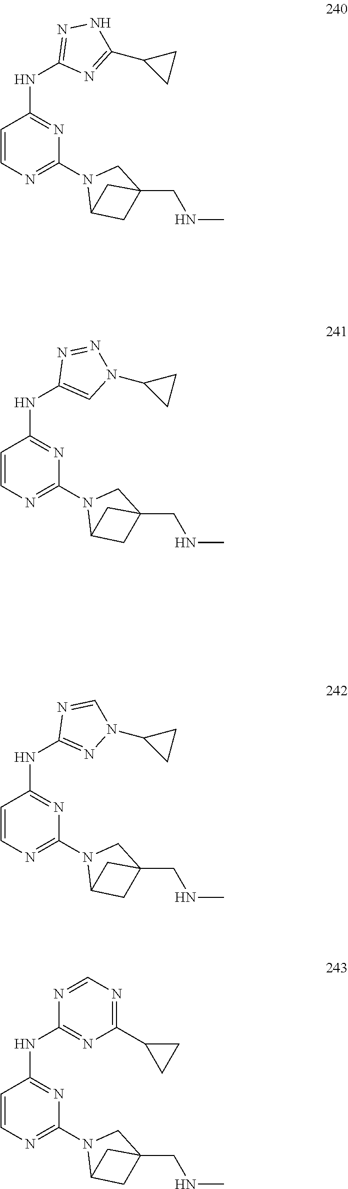 [SMILES: CNCC12CC(C1)N(c1nccc(Nc3n[nH]c(C4CC4)n3)n1)C2 ; CNCC12CC(C1)N(c1nccc(Nc3cn(C4CC4)nn3)n1)C2 ; CNCC12CC(C1)N(c1nccc(Nc3ncn(C4CC4)n3)n1)C2 ; CNCC12CC(C1)N(c1nccc(Nc3ncnc(C4CC4)n3)n1)C2] 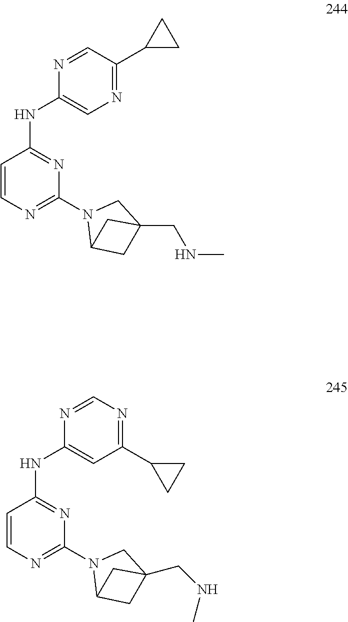 [SMILES: CNCC12CC(C1)N(c1nccc(Nc3cnc(C4CC4)cn3)n1)C2 ; CNCC12CC(C1)N(c1nccc(Nc3cc(C4CC4)ncn3)n1)C2]
or a pharmaceutically acceptable salt, stereoisomer, and/or N-oxide thereof.

32. A compound of Formula III: 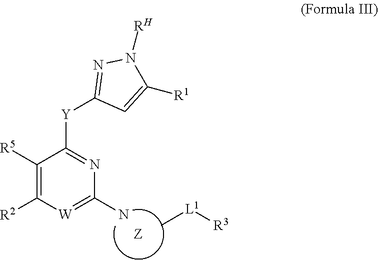
or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
ring Z is selected from the group consisting of 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁-C₄ alkyl, oxo, C₁-C₄alkoxy, and cyano;
L¹ is bond or C₁-C₆ alkylene (optionally substituted by one, two or three R⁴);
R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
R⁴ is each independently selected from the group consisting of halogen and C₁-C₆ alkyl, or two R⁴ together with the carbon from which the R⁴ are attached form a C₃-C₅ carbocyclyl;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R^{N} is selected from the group consisting of H and C₁-C₃alkyl, or two R^{N} together with the nitrogen atoms to which the R^{N}s are attached form a 4-6 membered heterocyclic ring;
R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
R² is selected from the group consisting of H, F, CF₃, —C(O)—O-methyl, —C(O)OH, —O-methyl, —O-ethyl, C₃-C₇ cycloalkyl, and heterocyclyl, and
R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring.

33. The compound of claim 32, wherein W is N, and having the Formula IIIa: 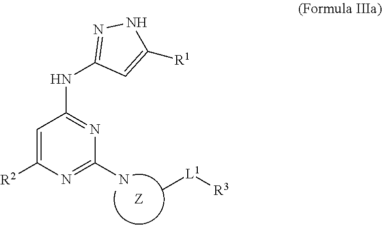
or a pharmaceutically acceptable salt, stereoisomer and/or N-oxide thereof.

34. The compound of any one of claims 31-33, wherein R¹ is a 5-6 membered heterocyclyl or C₃-C₆ cycloalkyl.

35. The compound of any one of claims 31-34, wherein R¹ is a C₃-C₆ cycloalkyl.

36. The compound of any one of claims 31-35, wherein R¹ is cyclopropyl.

37. The compound of any one of claims 31-35, wherein R¹ is cyclopentyl.

38. The compound of any one of claims 31-37, wherein ring Z is selected from the group consisting of:

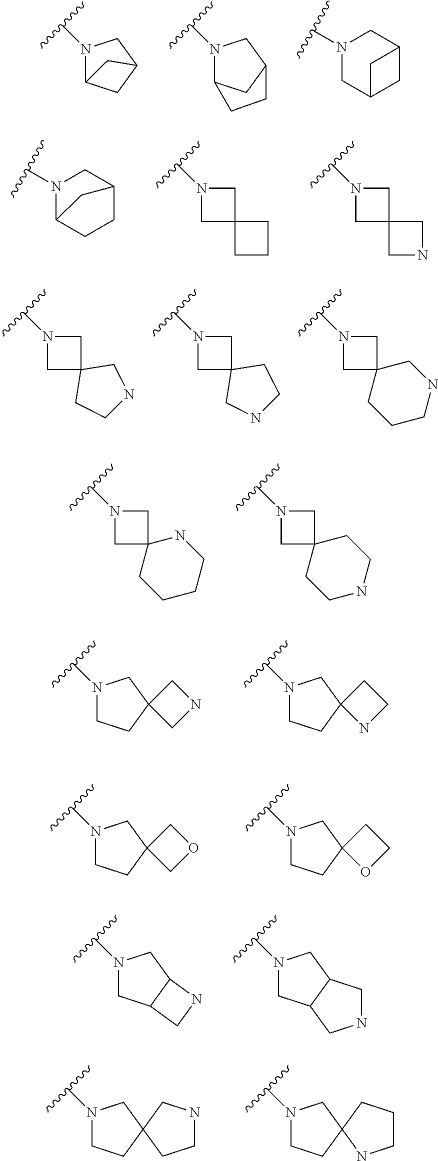

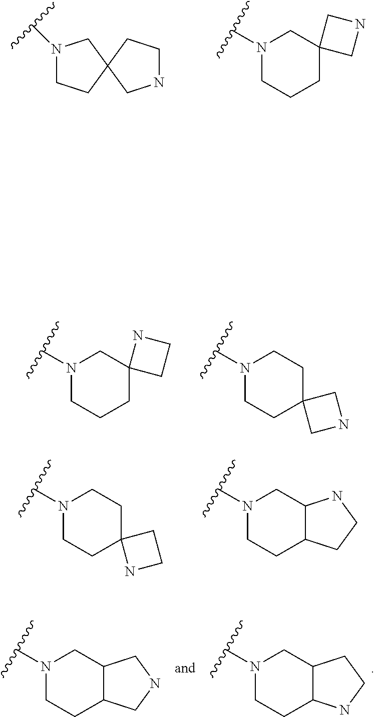

39. The compound of any one of claims 31-38, wherein L¹ is bond.

40. The compound of any one of claims 31-38, wherein L¹ is C₁-C₆ alkylene.

41. The compound of claim 40, wherein L¹ is methylene.

42. The compound of any one of claims 31-38, wherein L¹ is C₁-C₄ alkylene substituted by one or two R⁴.

43. The compound of any one of claims 31-38 and 42, wherein L¹ is methylene substituted by one or two R⁴.

44. The compound of claim 43, wherein R⁴ is C₁-C₃ alkyl (optionally substituted by one, two or three halogens).

45. The compound of claim 43, wherein two R⁴s together with the carbon to which the R⁴ are attached form a C₃-C₄ carbocyclyl.

46. The compound of any one of claims 31-45, wherein R³ is H or C₁-C₆ alkyl.

47. The compound of claim 46, wherein R³ is H or methyl.

48. The compound of any one of claims 31-45, wherein R³ is NR′R′.

49. The compound of claim 48, wherein R³ is NH₂, NH(CH₃), or N(CH₃)₂.

50. The compound of any one of claims 31-45, wherein R³ is 4-6 membered heterocyclyl optionally substituted by one or two substituents each independently selected from the group consisting of halo, hydroxyl, C₁-C₄ alkyl (optionally substituted by one, two or three halogens), and oxo.

51. The compound of any one of claims 31-50, wherein R² is H or F.

52. The compound of any one of claims 31-51, wherein R² is H.

53. The compound of claim 31 or 32, wherein R⁵ is H or methyl.

54. The compound of any one of claims 31, 32, and 53, wherein R⁵ is H.

55. A compound of Formula VI: 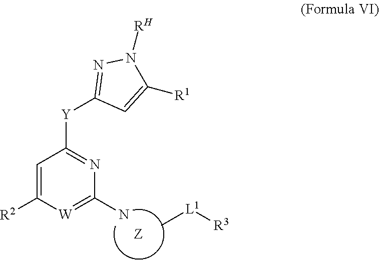
or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CF₂, CH(CH₃), C(CH₃)₂, and C(CH₂CH₂);
ring Z is a 4-10 membered N-bound heterocyclic, wherein Z may optionally be substituted by one or two substituents each independently selected from the group consisting of halo, hydroxyl, C₁-C₄ alkyl (optionally substituted by one, two or three halogens), and oxo;
L¹ is bond or C₁-C₆ alkylene (optionally substituted by one, two or three halogens);
R³ is selected from the group consisting of H, C₁-C₆ alkyl, NR′R′, and heterocyclyl optionally substituted by one or two substituents each independently selected from the group consisting of halo, hydroxyl, C₁-C₄ alkyl (optionally substituted by one, two or three halogens), and oxo;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R¹ is selected from the group consisting of C₃-C₁₀ cycloalkyl, spiro C₅-C₁₀ bicycloalkyl, heterocyclyl, cyano, halo, and heteroaryl; wherein C₃-C₁₀ cycloalkyl, heterocyclyl, or heteroaryl may be substituted by one, two or three substituents each independently selected from halo and C₁-C₄alkyl (optionally substituted by one, two or three halogens); and
R² is selected from the group consisting of H, F, CF₃, —C(O)—O-methyl, —C(O)OH, —O-methyl, C₃-C₇ cycloalkyl, and heterocyclyl;
wherein when ring Z is a 6 membered monocyclic heterocycle, L¹ is C₁-C₆alkylene or L¹ is a bond and R³ is selected from C₁-C₆alkyl and NHR′.
A compound is selected from

56. A method of treating a proliferative disease, comprising: administering to a subject with a proliferative disease a therapeutically effective amount of a compound of Formula IV: 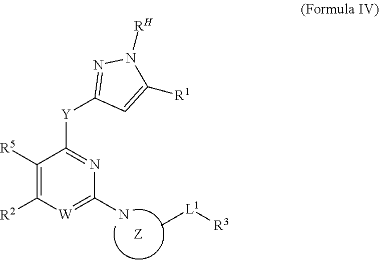
or a pharmaceutically acceptable salt, stereoisomer, and/or N-oxide thereof, wherein:
W is selected from the group consisting of N, C—H, and C—F;
Y is selected from the group consisting of NH, N—CH₃, O, S, CH₂, CFH, CF₂, CH(CH₃), C(CH₃)₂, C(CH₂CH₂), CHCF₃, CHCF₂H, CH(OCF₂H), CHOH, C(CH₃)OH, and CH(OCH₃);
ring Z is a 4-10 membered N-bound heterocyclic, wherein Z may optionally be substituted by one or two substituents each independently selected from the group consisting of halo, hydroxyl, C₁-C₄ alkyl (optionally substituted by one, two or three halogens), oxo, C₁-C₄alkoxy, and cyano;
L¹ is bond or C₁-C₆ alkylene (optionally substituted by one, two or three R⁴);
R³ is selected from the group consisting of H, C₁-C₆ alkyl, NR′R′, S(O)₀₋₂C₁-C₄alkyl, SO₂N(R^{N})₂, N(R^{N})SO₂—C₁-C₄alkyl, and heterocyclyl optionally substituted by one or two substituents each independently selected from the group consisting of halo, hydroxyl, C₁-C₄ alkyl (optionally substituted by one, two or three halogens), C₁-C₄ alkoxy, and oxo;
R⁴ is each independently selected from the group consisting of halogen and C₁-C₆ alkyl, or two R⁴ together with the carbon from which the R⁴ are attached form a C₃-C₅ carbocyclyl;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo;
R^{H} is selected from the group consisting of H, C₁-C₃alkyl, —C(O)—C₁-C₃alkyl, and C(O)—O—C₁-C₃alkyl;
R^{N} is selected from the group consisting of H and C₁-C₃alkyl, or two R^{N} together with the nitrogen atoms to which the R^{N}s are attached form a 4-6 membered heterocyclic ring;
R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens);
R² is selected from the group consisting of H, F, CF₃, —C(O)—O-methyl, —C(O)OH, —O-methyl, —O-ethyl, methyl, C₃-C₇ cycloalkyl, and heterocyclyl; and
R⁵ is selected from the group consisting of H, halo, C₁-C₄ alkyl (optionally substituted by one, two or three halogens); or R² and R⁵ together with the carbon atoms to which R² and R⁵ are attached form a 5-6 membered carbocyclic ring.

57. The method of claim 56, wherein W is N, and having the Formula IVa: 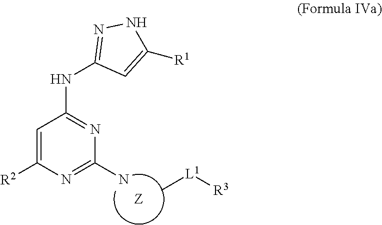
or a pharmaceutically acceptable salt, stereoisomer and/or N-oxide thereof.

58. The method of claim 56 or 57, wherein R¹ is a 5-6 membered heterocyclyl or C₃-C₆ cycloalkyl.

59. The method of any one of claims 56-58, wherein R¹ is a C₃-C₆ cycloalkyl.

60. The method of any one of claims 56-59, wherein R¹ is cyclopropyl.

61. The method of any one of claims 56-59, wherein R¹ is cyclopentyl.

62. The method of any one of claims 56-61, wherein ring Z is selected from the group consisting of 4-6 membered monocyclic heterocycle, 6-10 membered spiroheterocycle, 6-10 membered fused bicyclic heterocycle, and 6-10 membered bridged heterocycle, wherein Z may optionally be substituted by one or two substituents each independently selected from the group consisting of halo, hydroxyl, C₁-C₄ alkyl (optionally substituted by one, two or three halogens), and oxo.

63. The method of any one of claims 56-62, wherein ring Z is selected from the group consisting of 6-10 membered spiroheterocycle, a 6-10 membered fused bicyclic heterocyclic, and a 6-10 membered bridged cycloheteroalkyl, wherein Z may optionally be substituted by one or two substituents each independently selected from the group consisting of halo, hydroxyl, C₁-C₄ alkyl (optionally substituted by one, two or three halogens), and oxo.

64. The method of any one of claims 56-62, wherein ring Z is selected from the group consisting of:

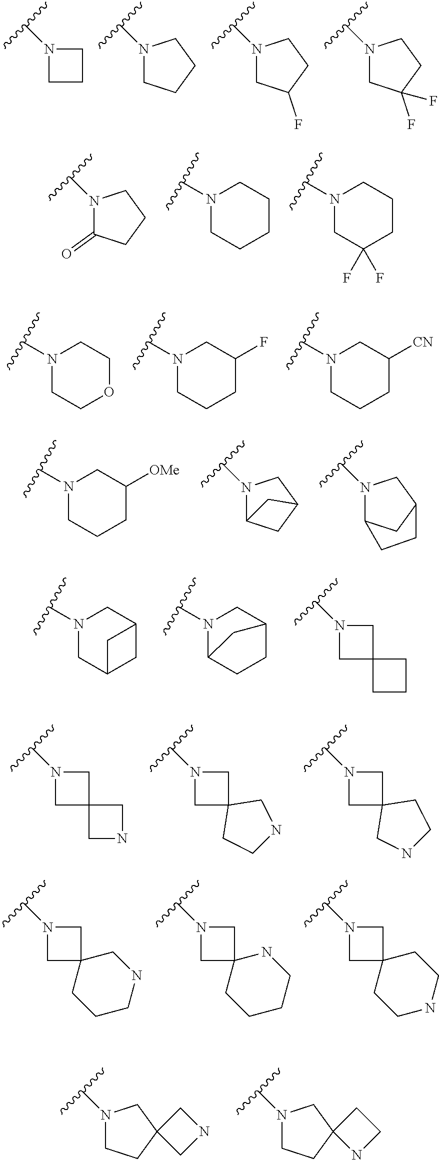

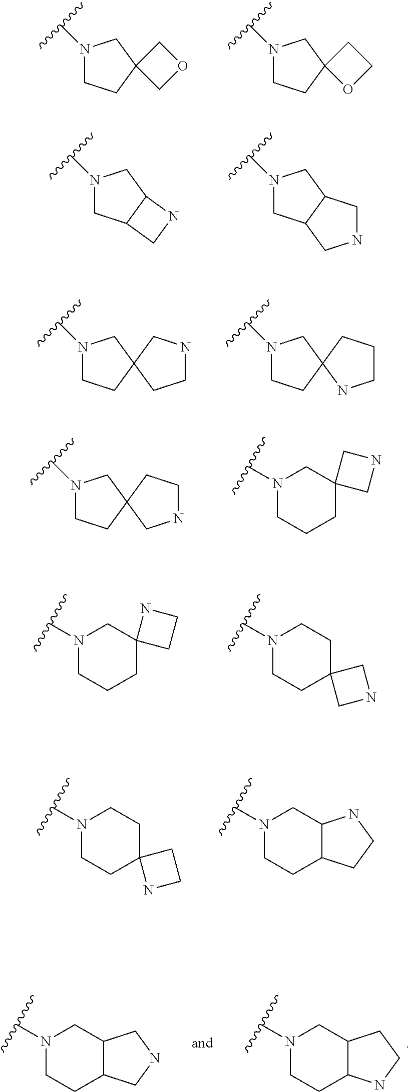

65. The method of any one of claims 56-64, represented by Formula IVb: 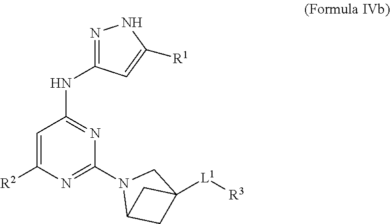
or a pharmaceutically acceptable salt, stereoisomer, and/or N-oxide thereof.

66. The method of any one of claims 56-64, represented by Formula IVc: 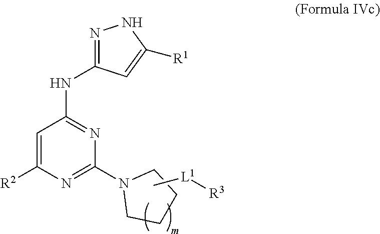
or a pharmaceutically acceptable salt, stereoisomer, and/or N-oxide thereof, wherein m is 0, 1, or 2.

67. The method of any one of claims 56-64, represented by Formula IVd: 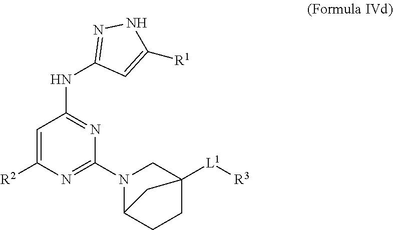
or a pharmaceutically acceptable salt, stereoisomer, and/or N-oxide thereof.

68. The method of any one of claims 56-64, represented by Formula IVe: 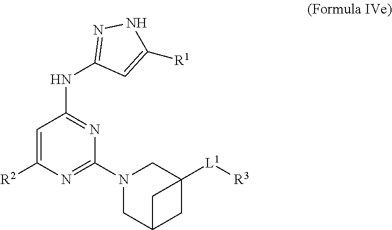
or a pharmaceutically acceptable salt, stereoisomer, and/or N-oxide thereof.

69. The method of any one of claims 56-68, wherein L¹ is bond.

70. The method of any one of claims 56-68, wherein L¹ is C₁-C₆ alkylene.

71. The method of claim 70, wherein L¹ is methylene.

72. The method of any one of claims 56-68, wherein L¹ is C₁-C₄ alkylene substituted by one or two R⁴.

73. The method of any one of claims 56-68 and 72, wherein L¹ is methylene substituted by one or two R⁴.

74. The method of claim 73, wherein R⁴ is C₁-C₃ alkyl (optionally substituted by one, two or three halogens).

75. The method of claim 73, wherein two R⁴s together with the carbon to which the R⁴ are attached form a C₃-C₄ carbocyclyl.

76. The method of any one of claims 56-75, wherein R³ is H or C₁-C₆ alkyl.

77. The method of claim 76, wherein R³ is H or methyl.

78. The method of any one of claims 56-75, wherein R³ is NR′R′.

79. The method of claim 78, wherein R³ is NH₂, NH(CH₃), or N(CH₃)₂.

80. The method of any one of claims 56-75, wherein R³ is 4-6 membered heterocyclyl optionally substituted by one or two substituents each independently selected from the group consisting of halo, hydroxyl, C₁-C₄ alkyl (optionally substituted by one, two or three halogens), and oxo.

81. The method of any one of claims 56-80, wherein R² is H, F, or methyl.

82. The method of any one of claims 56-81, wherein R² is H.

83. The method of claim 56, wherein R⁵ is H or methyl.

84. The method of claim 56 or 83 wherein R⁵ is H.

85. A compound of claim 55 selected from the group consisting of: 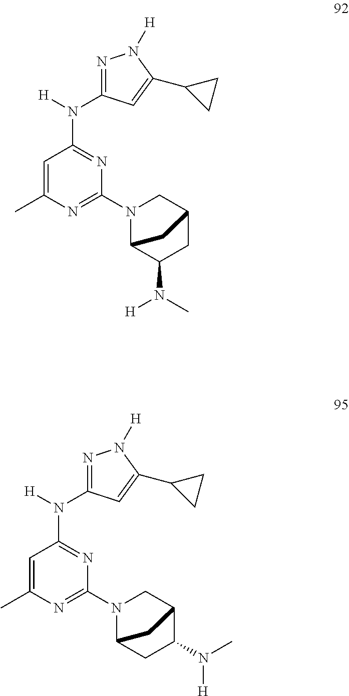
or a pharmaceutically acceptable salt stereoisomer, and/or N-oxide thereof.

86. A compound of Formula V: 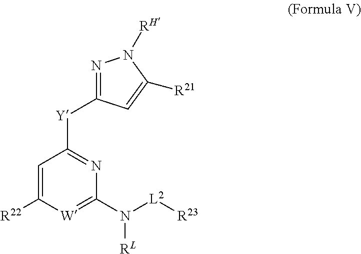
or a pharmaceutically acceptable salt, stereoisomer, and/or N-oxide thereof, wherein:
W′ is selected from the group consisting of N, C—H, and C—F;
Y′ is selected from the group consisting of NH, N—CH₃, O, S, CH₂, CFH, CF₂, CH(CH₃), C(CH₃)₂, C(CH₂CH₂). CHCF₃, CHCF₂H, CH(OCF₂H), CHOH, C(CH₃)OH, and CH(OCH₃);
R^{L} is H or C₁-C₆ alkyl;
L² is bond or C₁-C₆ alkylene (optionally substituted by one, two or three R²⁴);
R²³ is NR′R′;
R²⁴ is each independently selected from the group consisting of halogen and C₁-C₆ alkyl, or two R⁴ together with the carbon from which the R⁴ are attached form a C₃-C₅ carbocyclyl;
R′ for each occurrence is independently selected from the group consisting of H, C₁-C₆ alkyl (optionally substituted by one, two or three halogens), C₁-C₆ alkylene-O—C₁-C₄ alkyl, C₁-C₆ alkylene-SO₂—C₁-C₄ alkyl, and heterocyclyl optionally substituted by one or two substituents each independently selected from the group consisting of halo, hydroxyl, C₁-C₄ alkyl (optionally substituted by one, two or three halogens), and oxo; or two R's together with the nitrogen to which they are attached form a heterocyclyl which may optionally be substituted by methyl, halo, cyano, oxo, or hydroxyl;
R^{H′} is selected from the group consisting of H, C₁-C₃alkyl, —C(O)—C₁-C₃alkyl, and C(O)—O—C₁-C₃alkyl;
R²¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₁₀ cycloalkyl, spiro C₅-C₁₀ bicycloalkyl, heterocyclyl, or heteroaryl may be substituted by one, two or three substituents each independently selected from halo and C₁-C₄alkyl (optionally substituted by one, two or three halogens); and
R²² is selected from the group consisting of H, F, —C(O)—O-methyl, —C(O)OH, —O-methyl, —O-ethyl, methyl, C₃-C₇ cycloalkyl, and heterocyclyl.

87. The compound of claim 86, wherein W′ is N, and having the Formula Va: 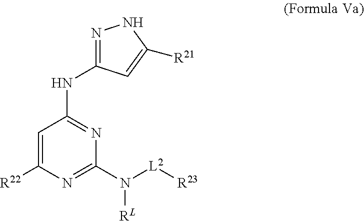
or a pharmaceutically acceptable salt, stereoisomer, and/or N-oxide thereof.

88. The compound of claim 85 or 86, wherein R²¹ is a 5-6 membered heterocyclyl or C₃-C₆ cycloalkyl.

89. The compound of any one of claims 85-87, wherein R²¹ is a C₃-C₆ cycloalkyl.

90. The compound of any one of claims 85-88, wherein R²¹ is cyclopropyl.

91. The compound of any one of claims 85-88, wherein R²¹ is cyclopentyl.

92. The compound of any one of claims 85-90, wherein L² is C₁-C₆ alkylene.

93. The compound of any one of claims 85-91, wherein L² is propylene.

94. The compound of any one of claims 85-92, wherein R²³ is NH₂, NH(CH₃), or N(CH₃)₂.

95. The compound of any one of claims 85-93, wherein two R's together with the nitrogen to which they are attached form a 4-6 membered heterocyclyl.

96. The compound of any one of claims 85-94, wherein R²² is H, F, or methyl.

97. The compound of any one of claims 85-95, wherein R²² is H.

98. The compound of claim 86 selected from the group consisting of: 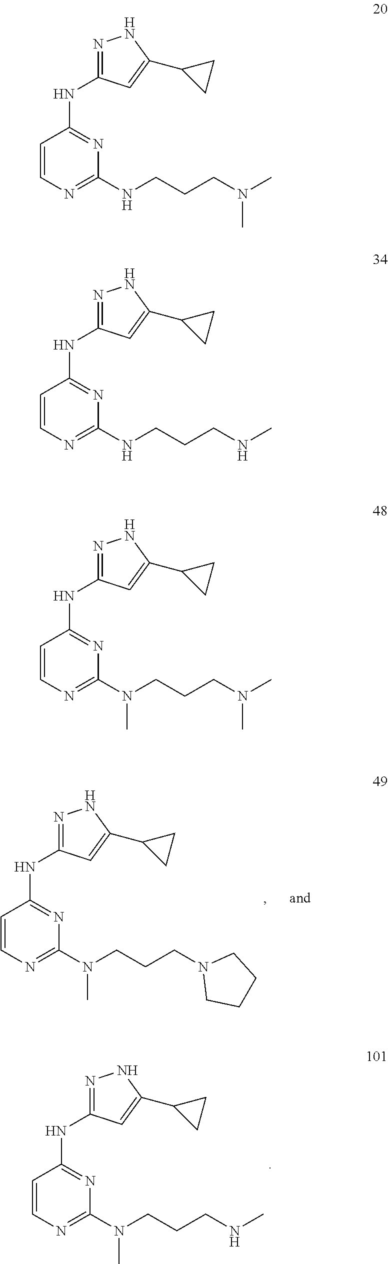 [SMILES: CN(C)CCCNc1nccc(Nc2cc(C3CC3)[nH]n2)n1 ; CNCCCNc1nccc(Nc2cc(C3CC3)[nH]n2)n1 ; CN(C)CCCN(C)c1nccc(Nc2cc(C3CC3)[nH]n2)n1 ; CN(CCCN1CCCC1)c1nccc(Nc2cc(C3CC3)[nH]n2)n1 ; CNCCCN(C)c1nccc(Nc2cc(C3CC3)[nH]n2)n1]
or a pharmaceutically acceptable salt, stereoisomer, and/or N-oxide thereof.

99. A pharmaceutical composition comprising a compound according to any one of claims 1-55 and 85-97, or a pharmaceutically acceptable salt, stereoisomer, and/or N-oxide thereof, and at least one pharmaceutically acceptable carrier or diluent.

100. The pharmaceutical composition of claim 98, wherein the composition is formulated for parenteral administration.

101. The pharmaceutical composition of claim 98, wherein the composition is formulated for intravenous administration.

102. The pharmaceutical composition of claim 98, wherein the composition is formulated for subcutaneous administration.

103. A method of treating a proliferative disease, comprising: administering to a subject with a proliferative disease a therapeutically effective amount of a compound according to any one of claims 1-55 and 85-97, or a pharmaceutically acceptable salt, stereoisomer and/or N-oxide thereof, or a therapeutically effective amount of the pharmaceutical composition of any one of claims 98-101.

104. The method of any one of claims 56-84 and 102, wherein the proliferative disease is cancer.

105. The method of claim 103, wherein the cancer is selected from the group consisting of head and neck cancer, nervous system cancer, brain cancer, neuroblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genito-urinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site.

106. A method of modulating MycN in cells of a subject in need thereof, comprising:
administering to a subject in need thereof an amount of a compound according to any one of claims 1-55 and 85-97, or a pharmaceutically acceptable salt, stereoisomer and/or N-oxide thereof, or a pharmaceutical composition according to any one of claims 98-101, that is effective to cause MycN modulation in cells of the subject.

107. The method of any one of claims 56-84 and 102-104, further comprising administering to the subject a second therapy.

108. The method of claim 106, wherein the second therapy is an antineoplastic therapy.

109. The method of claim 107, wherein the antineoplastic therapy is administration of one or more agents selected from a DNA topoisomerase I or II inhibitor, a DNA damaging agent, an immunotherapeutic agent, an antimetabolite or a thymidylate synthase (TS) inhibitor, a microtubule targeted agent, ionising radiation, an inhibitor of a mitosis regulator or a mitotic checkpoint regulator, an inhibitor of a DNA damage signal transducer, and an inhibitor of a DNA damage repair enzyme.

110. The method of claim 107, wherein the antineoplastic therapy is selected from the group consisting of immunotherapy, radiation therapy, photodynamic therapy, gene-directed enzyme prodrug therapy (GDEPT), antibody-directed enzyme prodrug therapy (ADEPT), gene therapy, and controlled diets.
